(19) United States
(12) Patent Application Publication
(10) Pub. No.: US 2024/0254559 A1
(43) Pub. Date: Aug. 1, 2024

(54) GENOMIC STABILITY PROFILING

(71) Applicant: Caris MPI, Inc., Irving, TX (US)

(72) Inventors: David Spetzler, Paradise Valley, AZ (US); Nianqing Xiao, Rockville, MD (US)

(21) Appl. No.: 18/396,565

(22) Filed: Dec. 26, 2023

Related U.S. Application Data
(63) Continuation of application No. 16/495,690, filed on Sep. 19, 2019, now abandoned.
(60) Provisional application No. 62/631,381, filed on Feb. 15, 2018.
(60) Provisional application No. 62/622,679, filed on Jan. 26, 2018.
(60) Provisional application No. 62/532,855, filed on Jul. 14, 2017.
(60) Provisional application No. 62/474,035, filed on Mar. 20, 2017.

(51) Int. Cl. C12Q 1/6886; C12Q 1/6827; G16B 20/10; G16B 20/20; G16B 30/00; G16B 30/10; G16H 10/40; G16H 20/10; G16H 50/20

(52) U.S. Cl. CPC C12Q 1/6886; C12Q 1/6827; G16H 10/40; G16H 20/10; G16H 50/20; C12Q 2600/156; G16B 20/10; G16B 20/20; G16B 30/00; G16B 30/10

(57) ABSTRACT

Provided herein are methods and systems of molecular profiling of diseases, such as cancer. In some embodiments, the molecular profiling can be used to identify treatments for the disease, such as treatments that provide potential benefit or potential lack of benefit for the disease. Molecular profiling can include biomarkers for immune checkpoint therapy, including microsatellite instability, tumor mutational burden, mismatch repair, and expression of checkpoint proteins such as PD-L1.

### CROSS-REFERENCE

This application is a continuation application of U.S. patent application Ser. No. 16/495,690, filed on Sep. 19, 2019, which claims the benefit of priority to International Application No. PCT/US2018/023438, filed on Mar. 20, 2018, which claims priority to U.S. Provisional Patent Application Nos. 62/474,035, filed on Mar. 20, 2017; 62/532,855, filed on Jul. 14, 2017; 62/622,679, filed on Jan. 26, 2018; and 62/631,381, filed on Feb. 15, 2018; which applications are incorporated by reference herein in their entirety.

### BACKGROUND

Disease states in patients are typically treated with treatment regimens or therapies that are selected based on clinical based criteria; that is, a treatment therapy or regimen is selected for a patient based on the determination that the patient has been diagnosed with a particular disease (which diagnosis has been made from classical diagnostic assays). Although the molecular mechanisms behind various disease states have been the subject of studies for years, the specific application of a diseased individual's molecular profile in determining treatment regimens and therapies for that individual has been disease specific and not widely pursued.

Some treatment regimens have been determined using molecular profiling in combination with clinical characterization of a patient such as observations made by a physician (such as a code from the International Classification of Diseases, for example, and the dates such codes were determined), laboratory test results, x-rays, biopsy results, statements made by the patient, and any other medical information typically relied upon by a physician to make a diagnosis in a specific disease. However, using a combination of selection material based on molecular profiling and clinical characterizations (such as the diagnosis of a particular type of cancer) to determine a treatment regimen or therapy presents a risk that an effective treatment regimen may be overlooked for a particular individual since some treatment regimens may work well for different disease states even though they are associated with treating a particular type of disease state.

Patients with refractory or metastatic cancer are of particular concern for treating physicians. The majority of patients with metastatic or refractory cancer eventually run out of treatment options or may suffer a cancer type with no real treatment options. For example, some patients have very limited options after their tumor has progressed in spite of front line, second line and sometimes third line and beyond) therapies. For these patients, molecular profiling of their cancer may provide the only viable option for prolonging life.

More particularly, additional targets or specific therapeutic agents can be identified assessment of a comprehensive number of targets or molecular findings examining molecular mechanisms, genes, gene expressed proteins, and/or combinations of such in a patient's tumor. Identifying multiple agents that can treat multiple targets or underlying mechanisms would provide cancer patients with a viable therapeutic alternative on a personalized basis so as to avoid standard therapies, which may simply not work or identify therapies that would not otherwise be considered by the treating physician.

There remains a need for better theranostic assessment of cancer victims, including molecular profiling analysis that provides more informed and effective personalized treatment options, resulting in improved patient care and enhanced treatment outcomes. The present invention provides methods and systems for identifying therapies of potential benefit and potential lack of benefit for these individuals by molecular profiling a sample from the individual. The molecular profiling can include analysis of genomic stability, including biomarkers that implicate immune checkpoint therapies. Such biomarkers include without limitation microsatellite instability (MSI), tumor mutational burden (TMB, also referred to as tumor mutation load or TML), mismatch repair proteins such as MLH1, MSH2, MSH6, and PMS2, immune modulating proteins such as PD-1, its ligand PD-L1, and CTLA-4.

### SUMMARY OF THE INVENTION

In an aspect, the invention provides a method of determining microsatellite instability (MSI) in a biological sample, comprising: (a) obtaining a nucleic acid sequence of a plurality of microsatellite loci from the biological sample; (b) determining the number of altered microsatellite loci based on the nucleic acid sequences obtained in step (a); (c) comparing the number of altered microsatellite loci determined in step (b) to a threshold number; and (d) identifying the biological sample as MSI-high if the number of altered microsatellite loci is greater than or equal to the threshold number.

In embodiments of the method of determining MSI, the biological sample comprises formalin-fixed paraffin-embedded (FFPE) tissue, fixed tissue, a core needle biopsy, a fine needle aspirate, unstained slides, fresh frozen (FF) tissue, formalin samples, tissue comprised in a solution that preserves nucleic acid or protein molecules, a fresh sample, a malignant fluid, a bodily fluid, a tumor sample, a tissue sample, or any combination thereof. In preferred embodiments, the biological sample comprises cells from a tumor, e.g., a solid tumor. The biological sample may comprise a bodily fluid. In some embodiments, the bodily fluid comprises a malignant fluid, a pleural fluid, a peritoneal fluid, or any combination thereof. In some embodiments, the bodily fluid comprises peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid, pre-ejaculatory fluid, female ejaculate, sweat, fecal matter, tears, cyst fluid, pleural fluid, peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyst cavity fluid, or umbilical cord blood.

In embodiments of the method of determining MSI, the nucleic acid sequence is obtained by sequencing DNA or RNA. In preferred embodiments, the DNA is genomic DNA. The sequencing can be high throughput sequencing (next generation sequencing (NGS)).

In embodiments of the method of determining MSI, the plurality of microsatellite loci comprises any useful number of loci, including without limitation at least 5, 10, 15, 20, 25, 30, 35, 40, 45, 50, 55, 60, 65, 70, 75, 80, 85, 90, 95, 100, 125, 150, 175, 200, 250, 300, 350, 400, 450, 500, 550, 600, 650, 700, 750, 800, 850, 900, 950, 1000, 2000, 3000, 4000, 5000, 6000, or 7000 loci. The plurality of microsatellite loci can be filtered to exclude loci meeting certain desired criteria. In preferred embodiments, the plurality of microsatellite loci excludes: i) sex chromosome loci; ii) microsatellite loci in regions that typically have lower coverage depth relative to other genomic regions; iii) microsatellites with repeat unit lengths greater than 3, 4, 5, 6 or 7 nucleotides, preferably greater than 5 nucleotides; or iv) any combination of i)-iii). In some embodiments, the members of the plurality of microsatellite loci are selected from Table 16. For examples, the plurality of microsatellite loci may comprise all loci in Table 16, or the plurality of loci may consist of all loci in Table 16. The members of the plurality of microsatellite loci can be chosen based on certain desired criteria. In some embodiments, each member of the plurality of microsatellite loci is located within the vicinity of a gene. In preferred embodiments, each member of the plurality of microsatellite loci is located within the vicinity of a cancer gene. For example, each member of the plurality of microsatellite loci can be located within the vicinity of a cancer gene selected from Table 7, Table 8, Table 9, Table 10, or any combination thereof.

In embodiments of the method of determining MSI, determining the number of altered microsatellite loci in step (b) comprises comparing each nucleic acid sequence obtained in step (a) to a reference sequence for each microsatellite loci. For example, the reference sequence can be a human genomic reference sequence, including without limitation the UCSC Genome Browser database. Determining the number of altered microsatellite loci may comprise identifying insertions or deletions that increased or decreased the number of repeats in each microsatellite loci. In some embodiments, the number of altered microsatellite loci only counts each altered loci once regardless of the number of insertions or deletions at that loci.

In embodiments of the method of determining MSI, the threshold number is calibrated based on comparison of the number of altered microsatellite loci per patient to MSI results obtained using a different laboratory technique on a same biological sample. The “same biological sample” can refer to any appropriate sample, such as the same physical sample or another portion of the same tumor. In some embodiments, the different laboratory technique comprises fragment analysis, immunohistochemistry of mismatch repair genes, immunohistochemistry of immunomodulators, or any combination thereof. In preferred embodiments, the different laboratory technique comprises the gold standard fragment analysis. The threshold number can be determined using any number of desired biological samples, including biological samples from at least 5, 10, 15, 20, 25, 30, 35, 40, 45, 50, 55, 60, 65, 70, 75, 80, 85, 90, 95, 100, 125, 150, 175, 200, 250, 300, 350, 400, 450, 500, 550, 600, 650, 700, 750, 800, 850, 900, 950, 1000, or 2000 different cancer patients. The samples can represent various cancers, e.g., from at least 1, 2, 3, 4, 5, 6, 7, 8, 9, 10, 11, 12, 13, 14, 15, 20, or 25 distinct cancer lineages. In some embodiments, the distinct cancer lineages comprise cancers selected from colorectal adenocarcinoma, endometrial cancer, bladder cancer, breast carcinoma, cervical cancer, cholangiocarcinoma, esophageal and esophagogastric junction carcinoma, extrahepatic bile duct adenocarcinoma, gastric adenocarcinoma, gastrointestinal stromal tumors, glioblastoma, liver hepatocellular carcinoma, lymphoma, malignant solitary fibrous tumor of the pleura, melanoma, neuroendocrine tumors, NSCLC, female genital tract malignancy, ovarian surface epithelial carcinomas, pancreatic adenocarcinoma, prostatic adenocarcinoma, small intestinal malignancies, soft tissue tumors, thyroid carcinoma, uterine sarcoma, uveal melanoma, and any combination thereof. In some embodiments, the threshold number is calibrated across at least 1, 2, 3, 4, 5, 6, 7, 8, 9, 10, 11, 12, 13, 14, 15, 20, or 25 distinct cancer lineages using sensitivity, specificity, positive predictive value, negative predictive value, or any combination thereof. For example, the threshold can be tuned with high sensitivity to MSI-high to reduce false negatives, or high specificity to MSI-high to reduce false positives, or any desired balance between. In a preferred embodiment, the threshold number is set to provide high sensitivity to MSI-high as determined in colorectal cancer using the different laboratory technique, wherein optionally the different laboratory technique comprises fragment analysis.

The threshold number can be expressed as a number of loci or a percentage of loci or any appropriate measure. In some embodiments, the threshold number is less than about 10%, 9%, 8%, 7%, 6%, 5%, 4%, 3%, 2%, 1%, 0.9%, 0.8%, 0.7%, 0.6%, 0.5%, 0.4%, 0.3%, 0.2%, or 0.1% of the number of members of the plurality of microsatellite loci. On the other hand, the threshold number can be greater than about 10%, 9%, 8%, 7%, 6%, 5%, 4%, 3%, 2%, 1%, 0.9%, 0.8%, 0.7%, 0.6%, 0.5%, 0.4%, 0.3%, 0.2%, or 0.1% of the number of members of the plurality of microsatellite loci. For example, the threshold number can be between about 10% and about 0.1% of the number of members of the plurality of microsatellite loci, or between about 5% and about 0.2% of the number of members of the plurality of microsatellite loci, or between about 3% and about 0.3% of the number of members of the plurality of microsatellite loci, or between about 1% and about 0.4% of the number of members of the plurality of microsatellite loci. As used herein, “about” may include a range of +/−10% of the stated value.

In an embodiment of the method of determining MSI, the number of members of the plurality of microsatellite loci is greater than 7000 and the threshold number is ≥40 and ≤50, wherein optionally the threshold level is 40, 41, 42, 43, 44, 45, 46, 47, 48, 49 or 50. As a non-limiting example, the members of the plurality of microsatellite loci can be those in Table 16, which comprises 7317 members, and the threshold can be set to 46 loci. In this example, the threshold is 0.63% of the number of members of the plurality of microsatellite loci. The threshold can be recalibrated as described herein with changing members of the plurality of microsatellite loci.

In preferred embodiments of the method of determining MSI, MSI status, e.g., high, stable or low, is determined without assessing microsatellite loci in normal tissue.

In embodiments of the method of determining MSI, the method further comprises identifying the biological sample as microsatellite stable (MSS) if the number of altered microsatellite loci is below the threshold number.

In embodiments of the method of determining MSI, the method further comprises identifying the biological sample as MSI-low if the number of altered microsatellite loci in the sample is less than or equal to a lower threshold number. As further described herein, the MSI-low can be calibrated using similar methodology as MSI high. MSS can be the range between MSI-high and MSH-low.

The invention provides a method of determining a tumor mutation burden (TMB; also referred to as tumor mutation load or TML) for a biological sample. In embodiments of the method of determining MSI, the method further comprises determining a tumor mutation burden (TMB) for the biological sample. In preferred embodiments, TMB is determined using the same laboratory analysis as MSI. As a non-limiting illustration, a NGS panel is run on a biological sample and the sequencing results are used to calculate MSI, TMB, or both. In some embodiments, TMB is determined by sequence analysis of a plurality of genes, including without limitation cancer genes selected from Table 7, Table 8, Table 9, Table 10, or any combination thereof. In a preferred embodiment, TMB is determined using missense mutations that have not been previously identified as germline alterations in the art. Similar to MSI-high, TMB-High can be determined by comparing a mutation rate to a TMB-High threshold, wherein TMB-High is defined as the mutation rate greater than or equal to the TMB-High threshold. The mutation rate can be expressed in any appropriate units, including without limitation units of mutations/megabase. The TMB-High threshold can be determined by comparing TMB with MSI determined in colorectal cancer from a same sample. In various embodiments, the TMB-High threshold is greater than or equal to 5, 6, 7, 8, 9, 10, 11, 12, 13, 14, 15, 16, 17, 18, 19, or 20 mutations/megabase of missense mutations. In a preferred embodiment, the TMB-High threshold is 17 mutations/megabase. Similarly, TMB-Low status can be determined by comparing a mutation rate to a TMB-Low threshold, wherein TMB-Low is defined as the mutation rate less than or equal to the TMB-Low threshold. The TMB-Low threshold can also be determined by comparing TMB with MSI determined in colorectal cancer from a same sample. In various embodiments, the TMB-Low threshold is less than or equal to 15, 14, 13, 12, 11, 10, 9, 8, 7, 6, 5, 4, 3, 2, or 1 mutations/megabase of missense mutations. In a preferred embodiment, the TMB-Low threshold is 6 mutations/megabase.

In embodiments of the method of determining MSI, TMB, or both, the method further comprises profiling various additional biomarkers in the biological sample as desired, e.g., mismatch repair proteins such as MLH1, MSH2, MSH6, and PMS2, immune checkpoint protein such as PD-L1, or any combination thereof. The profiling can comprise any useful technique, including without limitation determining: i) a protein expression level, wherein optionally the protein expression level is determined using IHC, flow cytometry or an immunoassay; ii) a nucleic acid sequence, wherein optionally the sequence is determined using next generation sequencing; iii) a promoter hypermethylation, wherein optionally the hypermethylation is determined using pyrosequencing; and iv) any combination thereof.

In another aspect, the invention provides a method of identifying at least one therapy of potential benefit for an individual with cancer, the method comprising: (a) obtaining the biological sample from the individual, e.g., as described herein; (b) generating a molecular profile by performing the method of the invention for determining MSI, TMB, or both on the biological sample; and (c) identifying the therapy of potential benefit based on the molecular profile. Generating the molecular profile can also comprise performing additional analysis on the biological sample according to Table 5, Table 6, Table 7, Table 8, Table 9, Table 10, or any combination thereof. In some embodiments, generating the molecular profile comprises performing additional analysis on the biological sample to: i) determine a tumor mutation burden (TMB); ii) determine an expression level of MLH1; iii) determine an expression level of MSH2, determine an expression level of MSH6; iv) determine an expression level of PMS2; v) determine an expression level of PD-L1; vi) or any combination thereof. The step of identifying can use drug-biomarker associations, such as those described herein. See, e.g., Table 11. In a preferred embodiment, the step of identifying comprises identifying potential benefit from an immune checkpoint inhibitor therapy when the biological sample is MSI-High. Similarly, the step of identifying may comprise identifying potential benefit from an immune checkpoint inhibitor therapy when the biological sample is MSI-High, TMB-High, MLH1-, MSH2-, MSH6-, PMS2-, PD-L1+, or any combination thereof. The step of identifying may comprise identifying potential benefit from an immune checkpoint inhibitor therapy when the biological sample is MSI-High, TMB-High, PD-L1+, or any combination thereof. See, e.g., Example 8 herein, which notes that each of these biomarkers can provide independent information; see also FIGS. 27A-BR and related text. The method can identify any useful immune checkpoint inhibitor therapy, including without limitation ipilimumab, nivolumab, pembrolizumab, atezolizumab, avelumab, durvalumab, pidilizumab, AMP-224, AMP-514, PDR001, BMS-936559, or any combination thereof. In addition, the method may comprise identifying at least one therapy of potential lack of benefit based on the molecular profile, at least one clinical trial for the subject based on the molecular profile, or any combination thereof. For examples, see FIGS. 27A-BR.

In embodiments of the method of identifying at least one therapy of potential benefit, the subject has not previously been treated with the at least one therapy of potential benefit. The cancer may comprise a metastatic cancer, a recurrent cancer, or any combination thereof. In some cases, the cancer is refractory to a prior therapy, including without limitation front-line or standard of care therapy for the cancer. In some embodiments, the cancer is refractory to all known standard of care therapies. In other embodiments, the subject has not previously been treated for the cancer. The method may further comprise administering the at least one therapy of potential benefit to the individual. Progression free survival (PFS), disease free survival (DFS), or lifespan can be extended by the administration.

The method of identifying at least one therapy of potential benefit can be employed for any desired cancer. In various embodiments, the cancer comprises an acute lymphoblastic leukemia; acute myeloid leukemia; adrenocortical carcinoma; AIDS-related cancer; AIDS-related lymphoma; anal cancer; appendix cancer; astrocytomas; atypical teratoid/rhabdoid tumor; basal cell carcinoma; bladder cancer; brain stem glioma; brain tumor, brain stem glioma, central nervous system atypical teratoid/rhabdoid tumor, central nervous system embryonal tumors, astrocytomas, craniopharyngioma, ependymoblastoma, ependymoma, medulloblastoma, medulloepithelioma, pineal parenchymal tumors of intermediate differentiation, supratentorial primitive neuroectodermal tumors and pineoblastoma; breast cancer; bronchial tumors; Burkitt lymphoma; cancer of unknown primary site (CUP); carcinoid tumor; carcinoma of unknown primary site; central nervous system atypical teratoid/rhabdoid tumor; central nervous system embryonal tumors; cervical cancer; childhood cancers; chordoma; chronic lymphocytic leukemia; chronic myelogenous leukemia; chronic myeloproliferative disorders; colon cancer; colorectal cancer; craniopharyngioma; cutaneous T-cell lymphoma; endocrine pancreas islet cell tumors; endometrial cancer; ependymoblastoma; ependymoma; esophageal cancer; esthesioneuroblastoma; Ewing sarcoma; extracranial germ cell tumor; extragonadal germ cell tumor; extrahepatic bile duct cancer; gallbladder cancer; gastric (stomach) cancer; gastrointestinal carcinoid tumor; gastrointestinal stromal cell tumor; gastrointestinal stromal tumor (GIST); gestational trophoblastic tumor; glioma; hairy cell leukemia; head and neck cancer; heart cancer; Hodgkin lymphoma; hypopharyngeal cancer; intraocular melanoma; islet cell tumors; Kaposi sarcoma; kidney cancer; Langerhans cell histiocytosis; laryngeal cancer; lip cancer; liver cancer; malignant fibrous histiocytoma bone cancer; medulloblastoma; medulloepithelioma; melanoma; Merkel cell carcinoma; Merkel cell skin carcinoma; mesothelioma; metastatic squamous neck cancer with occult primary; mouth cancer; multiple endocrine neoplasia syndromes; multiple myeloma; multiple myeloma/plasma cell neoplasm; mycosis fungoides; myelodysplastic syndromes; myeloproliferative neoplasms; nasal cavity cancer; nasopharyngeal cancer; neuroblastoma; Non-Hodgkin lymphoma; nonmelanoma skin cancer; non-small cell lung cancer; oral cancer; oral cavity cancer; oropharyngeal cancer; osteosarcoma; other brain and spinal cord tumors; ovarian cancer; ovarian epithelial cancer; ovarian germ cell tumor; ovarian low malignant potential tumor; pancreatic cancer; papillomatosis; paranasal sinus cancer; parathyroid cancer; pelvic cancer; penile cancer; pharyngeal cancer; pineal parenchymal tumors of intermediate differentiation; pineoblastoma; pituitary tumor; plasma cell neoplasm/multiple myeloma; pleuropulmonary blastoma; primary central nervous system (CNS) lymphoma; primary hepatocellular liver cancer; prostate cancer; rectal cancer; renal cancer; renal cell (kidney) cancer; renal cell cancer; respiratory tract cancer; retinoblastoma; rhabdomyosarcoma; salivary gland cancer; Sdzary syndrome; small cell lung cancer; small intestine cancer; soft tissue sarcoma; squamous cell carcinoma; squamous neck cancer; stomach (gastric) cancer; supratentorial primitive neuroectodermal tumors; T-cell lymphoma; testicular cancer; throat cancer; thymic carcinoma; thymoma; thyroid cancer; transitional cell cancer; transitional cell cancer of the renal pelvis and ureter; trophoblastic tumor; ureter cancer; urethral cancer; uterine cancer; uterine sarcoma; vaginal cancer; vulvar cancer; Waldenstrdm macroglobulinemia; or Wilm's tumor. In various embodiments, the cancer comprises an acute myeloid leukemia (AML), breast carcinoma, cholangiocarcinoma, colorectal adenocarcinoma, extrahepatic bile duct adenocarcinoma, female genital tract malignancy, gastric adenocarcinoma, gastroesophageal adenocarcinoma, gastrointestinal stromal tumor (GIST), glioblastoma, head and neck squamous carcinoma, leukemia, liver hepatocellular carcinoma, low grade glioma, lung bronchioloalveolar carcinoma (BAC), non-small cell lung cancer (NSCLC), lung small cell cancer (SCLC), lymphoma, male genital tract malignancy, malignant solitary fibrous tumor of the pleura (MSFT), melanoma, multiple myeloma, neuroendocrine tumor, nodal diffuse large B-cell lymphoma, non epithelial ovarian cancer (non-EOC), ovarian surface epithelial carcinoma, pancreatic adenocarcinoma, pituitary carcinomas, oligodendroglioma, prostatic adenocarcinoma, retroperitoneal or peritoneal carcinoma, retroperitoneal or peritoneal sarcoma, small intestinal malignancy, soft tissue tumor, thymic carcinoma, thyroid carcinoma, or uveal melanoma. The cancer can be of a lineage listed in Table 19.

In a related aspect, the invention provides a method of generating a molecular profiling report comprising preparing a report comprising the generated molecular profile using the methods of the invention above. In some embodiments, the report further comprises a list of the at least one therapy of potential benefit for the individual. In some embodiments, the report further comprises a list of at least one therapy of potential lack of benefit for the individual. In some embodiments, the report further comprises a list of at least one therapy of indeterminate benefit for the individual. The report may comprise identification of the at least one therapy as standard of care or not for the cancer lineage. The report can also comprise a listing of biomarkers tested when generating the molecular profile, the type of testing performed for each biomarker, and results of the testing for each biomarker. In some embodiments, the report further comprises a list of clinical trials for which the subject is indicated and/or eligible based on the molecular profile. In some embodiments, the report further comprises a list of evidence supporting the identification of therapies as of potential benefit, potential lack of benefit, or indeterminate benefit based on the molecular profile. The report can comprise any or all of these elements. For example, the report may comprise: 1) a list of biomarkers tested in the molecular profile; 2) a description of the molecular profile of the biomarkers as determined for the subject (e.g., type of testing and result for each biomarker); 3) a therapy associated with at least one of the biomarkers in the molecular profile; and 4) and an indication whether each therapy is of potential benefit, potential lack of benefit, or indeterminate benefit for treating the individual based on the molecular profile. The description of the molecular profile of the biomarkers can include the technique used to assess the biomarkers and the results of the assessment. The report can be computer generated, and can be a printed report, a computer file or both. The report can be made accessible via a secure web portal.

In an aspect, the invention provides the report generated by the methods of the invention. In a related aspect, the invention provides a computer system for generating the report. Exemplary reports generated according to the methods of the invention, and generated by a system of the invention, are found herein in FIGS. 27A-BR.

In an aspect, the invention provides use of a reagent in carrying out the methods of the invention as described above. In a related aspect, the invention provides of a reagent in the manufacture of a reagent or kit for carrying out the methods of the invention as described above. In still another related aspect, the invention provides a kit comprising a reagent for carrying out the methods of the invention as described above. The reagent can be any useful and desired reagent. In preferred embodiments, the reagent comprises at least one of a reagent for extracting nucleic acid from a sample, a reagent for performing ISH, a reagent for performing IHC, a reagent for performing PCR, a reagent for performing Sanger sequencing, a reagent for performing next generation sequencing, a probe set for performing next generation sequencing, a probe set for sequencing the plurality of microsatellite loci, a reagent for a DNA microarray, a reagent for performing pyrosequencing, a nucleic acid probe, a nucleic acid primer, an antibody, an aptamer, a reagent for performing bisulfite treatment of nucleic acid, and any combination thereof.

In an aspect, the invention provides a system for identifying at least one therapy associated with a cancer in an individual, comprising: (a) at least one host server; (b) at least one user interface for accessing the at least one host server to access and input data; (c) at least one processor for processing the inputted data; (d) at least one memory coupled to the processor for storing the processed data and instructions for: i) accessing an MSI status generated by the method of the invention above; and ii) identifying, based on the MSI status, at least one of: A) at least one therapy with potential benefit for treatment of the cancer; B) at least one therapy with potential lack of benefit for treatment of the cancer; and C) at least one therapy associated with a clinical trial; and (e) at least one display for displaying the identified at least one of: A) at least one therapy with potential benefit for treatment of the cancer; B) at least one therapy with potential lack of benefit for treatment of the cancer; and C) at least one therapy associated with a clinical trial. In some embodiments, the system further comprises at least one memory coupled to the processor for storing the processed data and instructions for identifying, based on the generated molecular profile according to the methods above, at least one of: A) at least one therapy with potential benefit for treatment of the cancer; B) at least one therapy with potential lack of benefit for treatment of the cancer; and C) at least one therapy associated with a clinical trial; and at least one display for display thereof. The system may further comprise at least one database comprising references for various biomarker states, data for drug/biomarker associations, or both. The at least one display can be a report provided by the invention.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

A better understanding of the features and advantages of the present invention will be obtained by reference to the following detailed description that sets forth illustrative embodiments, in which the principles of the invention are used, and the accompanying drawings of which:

FIG. 1 illustrates a block diagram of an exemplary embodiment of a system for determining individualized medical intervention for a particular disease state that utilizes molecular profiling of a patient's biological specimen that is non disease specific.

FIG. 2 is a flowchart of an exemplary embodiment of a method for determining individualized medical intervention for a particular disease state that utilizes molecular profiling of a patient's biological specimen that is non disease specific.

FIGS. 3A through 3D illustrate an exemplary patient profile report in accordance with step 80 of FIG. 2.

FIG. 4 is a flowchart of an exemplary embodiment of a method for identifying a drug therapy/agent capable of interacting with a target.

FIGS. 5-14 are flowcharts and diagrams illustrating various parts of an information-based personalized medicine drug discovery system and method in accordance with the present invention.

FIGS. 15-25 are computer screen print outs associated with various parts of the information-based personalized medicine drug discovery system and method shown in FIGS. 5-14.

FIGS. 26A-F illustrate a molecular profiling service requisition using a molecular profiling approach as outlined in Tables 5-11, and accompanying text herein.

FIGS. 27A-BR illustrate patient reports based on molecular profiling for individual patients having breast cancer (FIGS. 27A-Z), colorectal cancer (FIGS. 27AA-AV), or lung cancer (FIGS. 27AW-BR).

FIG. 28 illustrates a molecular profiling system that performs analysis of a cancer sample using a variety of components that measure expression levels, chromosomal aberrations and mutations. The molecular “blueprint” of the cancer is used to generate a prioritized ranking of druggable targets and/or drug associated targets in tumor and their associated therapies.

FIG. 29 shows an example output of microarray profiling results and calls made using a cutoff value.

FIG. 30 illustrates results of molecular profiling of PD1 and PDL1 in HPV+ and HPV-/TP53 mutated head and neck squamous cell carcinomas.

FIGS. 31A-C illustrate microsatellite instability analysis by Next Generation Sequencing (NGS).

FIGS. 32A-J illustrate microsatellite instability analysis by fragment analysis (FA), immunohistochemistry (IHC), and Next Generation Sequencing (NGS).

### DETAILED DESCRIPTION OF THE INVENTION

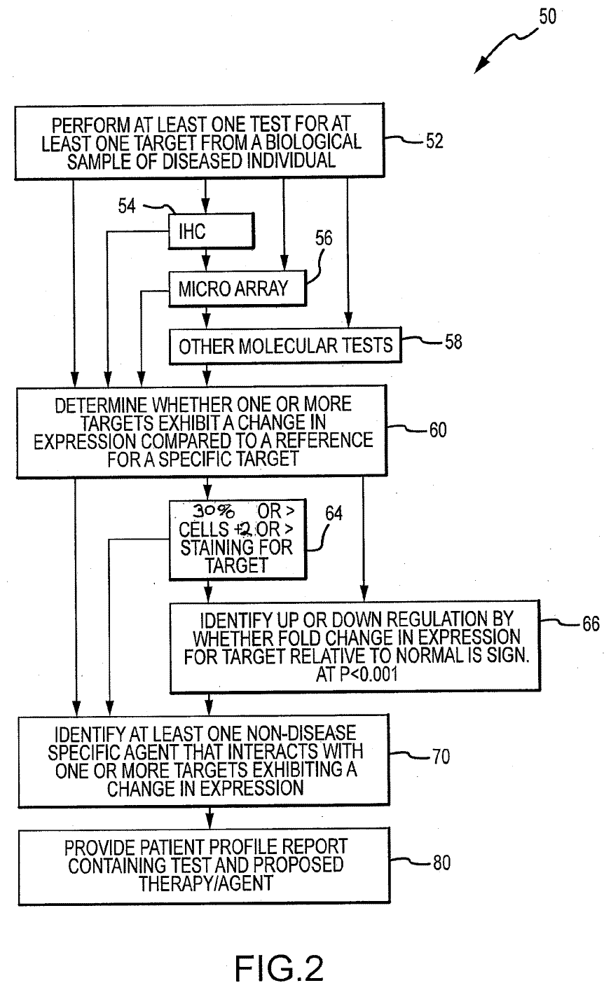

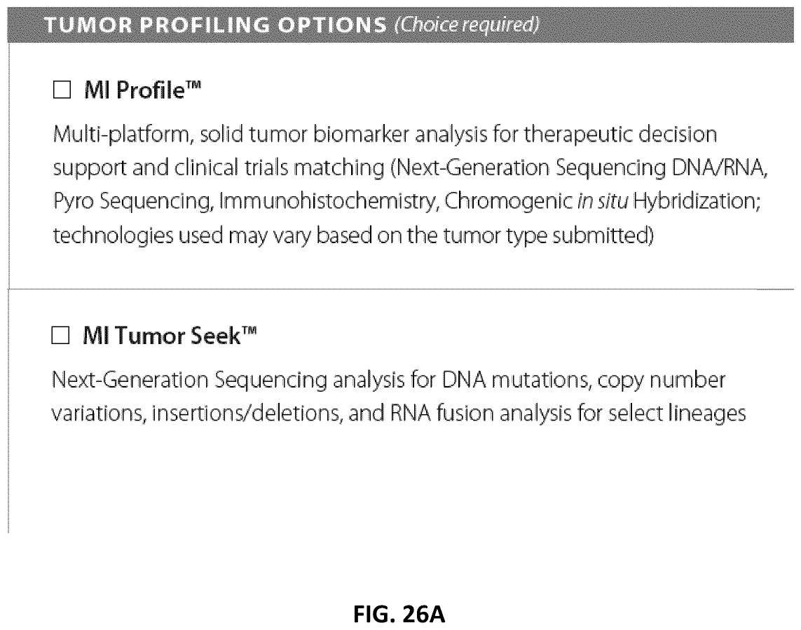

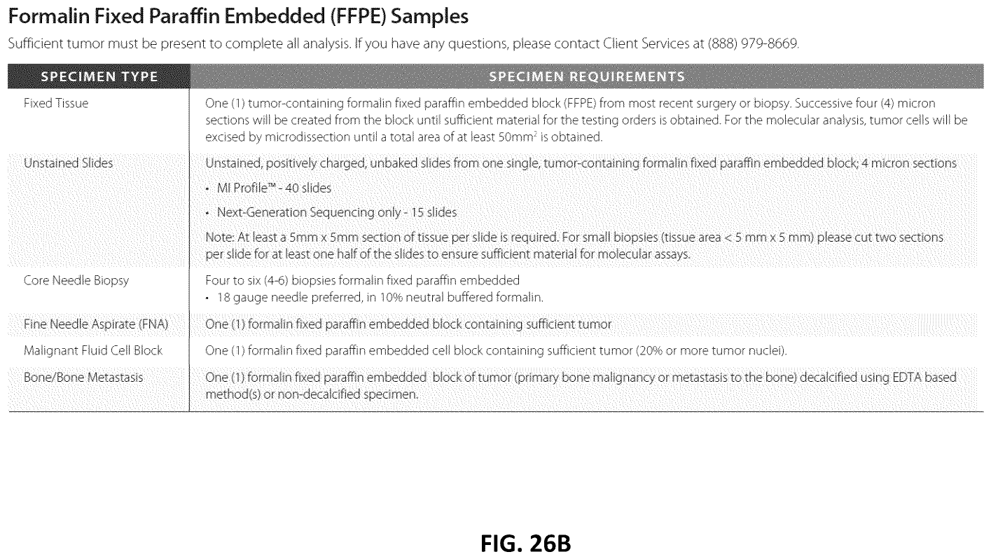

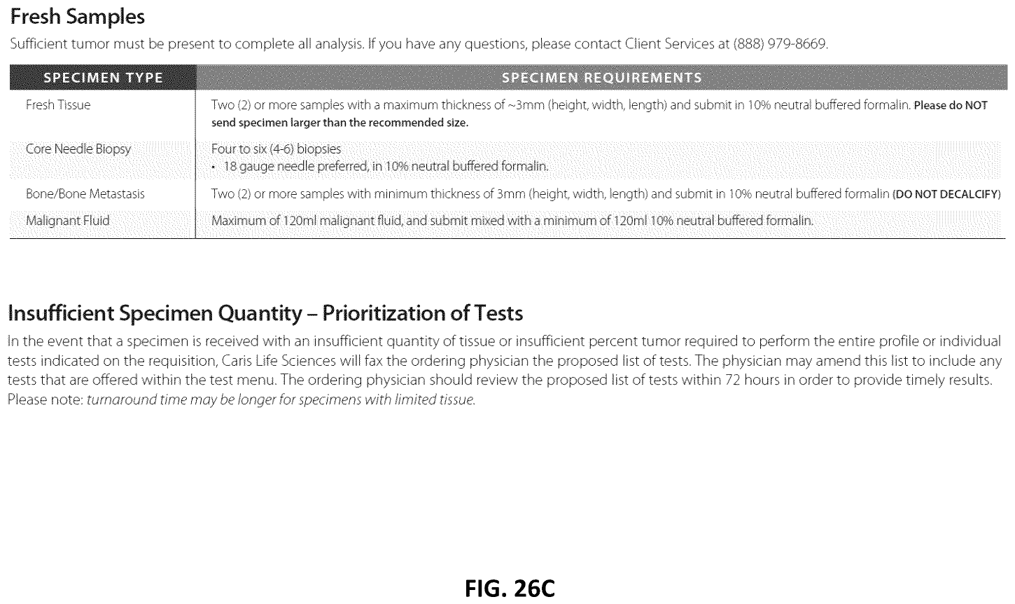

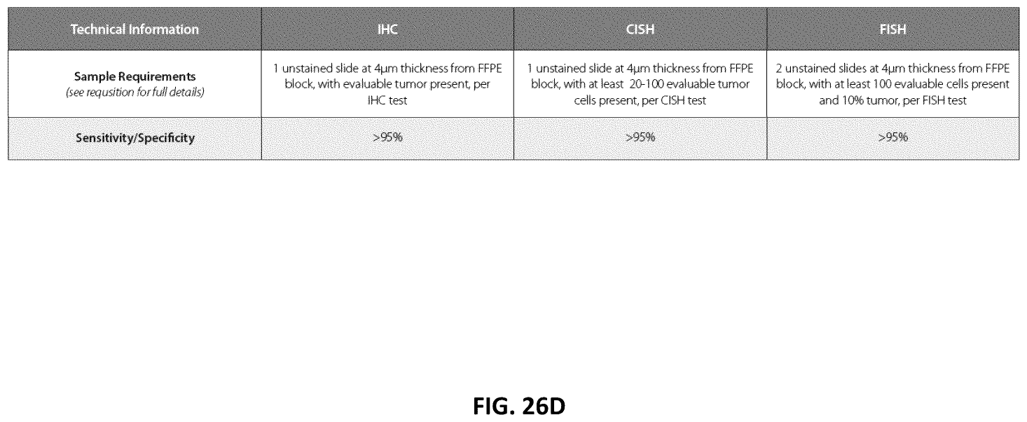

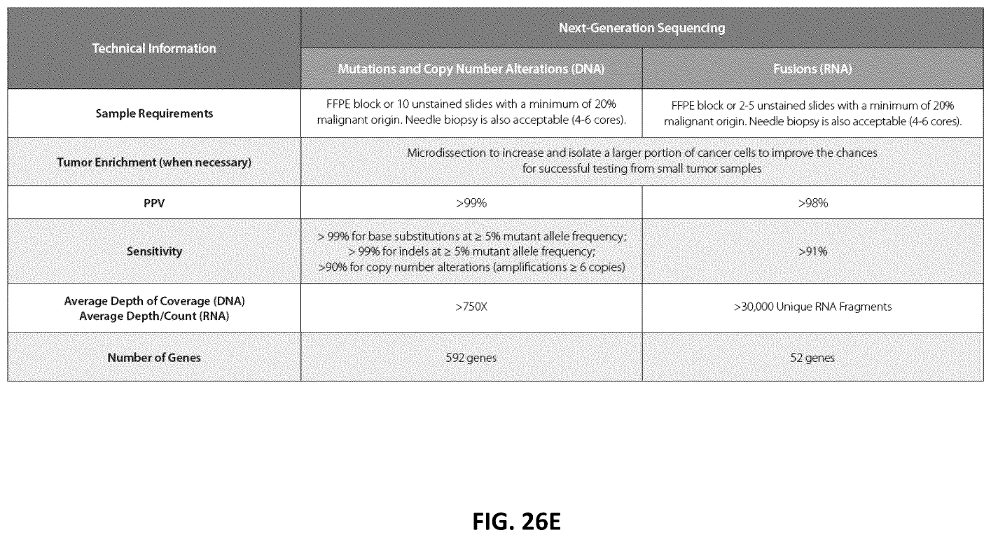

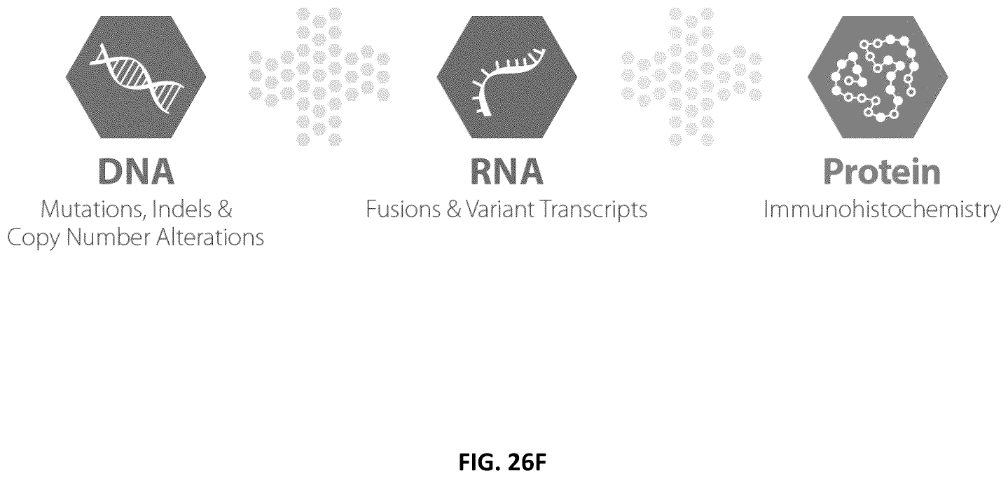

The present invention provides methods and systems for identifying therapeutic agents for use in treatments on an individualized basis by using molecular profiling. The molecular profiling approach provides a method for selecting a candidate treatment for an individual that could favorably change the clinical course for the individual with a condition or disease, such as cancer. The molecular profiling approach provides clinical benefit for individuals, such as identifying drug target(s) that provide a longer progression free survival (PFS), longer disease free survival (DFS), longer overall survival (OS) or extended lifespan. Methods and systems of the invention are directed to molecular profiling of cancer on an individual basis that can provide alternatives for treatment that may be convention or alternative to conventional treatment regimens. For example, alternative treatment regimes can be selected through molecular profiling methods of the invention where, a disease is refractory to current therapies, e.g., after a cancer has developed resistance to a standard-of-care treatment. Illustrative schemes for using molecular profiling to identify a treatment regime are provided in Tables 2-3, Table 11, FIGS. 2, 26A-F, and 28, which are each described in further detail herein. Molecular profiling provides a personalized approach to selecting candidate treatments that are likely to benefit a cancer. In embodiments, the molecular profiling method is used to identify therapies for patients with poor prognosis, such as those with metastatic disease or those whose cancer has progressed on standard front line therapies, or whose cancer has progressed on previous chemotherapeutic or hormonal regimens. The molecular profiling of the invention can also be used to guide treatment in the front-line setting as desired.

Personalized medicine based on pharmacogenetic insights, such as those provided by molecular profiling according to the invention, is increasingly taken for granted by some practitioners and the lay press, but forms the basis of hope for improved cancer therapy. However, molecular profiling as taught herein represents a fundamental departure from the traditional approach to oncologic therapy where for the most part, patients are grouped together and treated with approaches that are based on findings from light microscopy and disease stage. Traditionally, differential response to a particular therapeutic strategy has only been determined after the treatment was given, i.e. a posteriori. The “standard” approach to disease treatment relies on what is generally true about a given cancer diagnosis and treatment response has been vetted by randomized phase III clinical trials and forms the “standard of care” in medical practice. The results of these trials have been codified in consensus statements by guidelines organizations such as the National Comprehensive Cancer Network and The American Society of Clinical Oncology. The NCCN Compendium™ contains authoritative, scientifically derived information designed to support decision-making about the appropriate use of drugs and biologics in patients with cancer. The NCCN Compendium™ is recognized by the Centers for Medicare and Medicaid Services (CMS) and United Healthcare as an authoritative reference for oncology coverage policy. On-compendium treatments are those recommended by such guides. The biostatistical methods used to validate the results of clinical trials rely on minimizing differences between patients, and are based on declaring the likelihood of error that one approach is better than another for a patient group defined only by light microscopy and stage, not by individual differences in tumors. The molecular profiling methods of the invention exploit such individual differences. The methods can provide candidate treatments that can be then selected by a physician for treating a patient.

Molecular profiling can be used to provide a comprehensive view of the biological state of a sample. In an embodiment, molecular profiling is used for whole tumor profiling. Accordingly, a number of molecular approaches are used to assess the state of a tumor. The whole tumor profiling can be used for selecting a candidate treatment for a tumor. Molecular profiling can be used to select candidate therapeutics on any sample for any stage of a disease. In embodiment, the methods of the invention are used to profile a newly diagnosed cancer. The candidate treatments indicated by the molecular profiling can be used to select a therapy for treating the newly diagnosed cancer. In other embodiments, the methods of the invention are used to profile a cancer that has already been treated, e.g., with one or more standard-of-care therapy. In embodiments, the cancer is refractory to the prior treatment/s. For example, the cancer may be refractory to the standard of care treatments for the cancer. The cancer can be a metastatic cancer or other recurrent cancer. The treatments can be on-compendium or off-compendium treatments.

Molecular profiling can be performed by any known means for detecting a molecule in a biological sample. Molecular profiling comprises methods that include but are not limited to, nucleic acid sequencing, such as a DNA sequencing or RNA sequencing; immunohistochemistry (IHC); in situ hybridization (ISH); fluorescent in situ hybridization (FISH); chromogenic in situ hybridization (CISH); PCR amplification (e.g., qPCR or RT-PCR); various types of microarray (mRNA expression arrays, low density arrays, protein arrays, etc); various types of sequencing (Sanger, pyrosequencing, etc); comparative genomic hybridization (CGH); high throughput or next generation sequencing (NGS); Northern blot; Southern blot; immunoassay; and any other appropriate technique to assay the presence or quantity of a biological molecule of interest. In various embodiments of the invention, any one or more of these methods can be used concurrently or subsequent to each other for assessing target genes disclosed herein.

Molecular profiling of individual samples is used to select one or more candidate treatments for a disorder in a subject, e.g., by identifying targets for drugs that may be effective for a given cancer. For example, the candidate treatment can be a treatment known to have an effect on cells that differentially express genes as identified by molecular profiling techniques, an experimental drug, a government or regulatory approved drug or any combination of such drugs, which may have been studied and approved for a particular indication that is the same as or different from the indication of the subject from whom a biological sample is obtain and molecularly profiled.

When multiple biomarker targets are revealed by assessing target genes by molecular profiling, one or more decision rules can be put in place to prioritize the selection of certain therapeutic agent for treatment of an individual on a personalized basis. Rules of the invention aide prioritizing treatment, e.g., direct results of molecular profiling, anticipated efficacy of therapeutic agent, prior history with the same or other treatments, expected side effects, availability of therapeutic agent, cost of therapeutic agent, drug-drug interactions, and other factors considered by a treating physician. Based on the recommended and prioritized therapeutic agent targets, a physician can decide on the course of treatment for a particular individual. Accordingly, molecular profiling methods and systems of the invention can select candidate treatments based on individual characteristics of diseased cells, e.g., tumor cells, and other personalized factors in a subject in need of treatment, as opposed to relying on a traditional one-size fits all approach that is conventionally used to treat individuals suffering from a disease, especially cancer. In some cases, the recommended treatments are those not typically used to treat the disease or disorder inflicting the subject. In some cases, the recommended treatments are used after standard-of-care therapies are no longer providing adequate efficacy.

The treating physician can use the results of the molecular profiling methods to optimize a treatment regimen for a patient. The candidate treatment identified by the methods of the invention can be used to treat a patient; however, such treatment is not required of the methods. Indeed, the analysis of molecular profiling results and identification of candidate treatments based on those results can be automated and does not require physician involvement.

### Biological Entities

Nucleic acids include deoxyribonucleotides or ribonucleotides and polymers thereof in either single- or double-stranded form, or complements thereof. Nucleic acids can contain known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs). Nucleic acid sequence can encompass conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell Probes 8:91-98 (1994)). The term nucleic acid can be used interchangeably with gene, cDNA, mRNA, oligonucleotide, and polynucleotide.

A particular nucleic acid sequence may implicitly encompass the particular sequence and “splice variants” and nucleic acid sequences encoding truncated forms. Similarly, a particular protein encoded by a nucleic acid can encompass any protein encoded by a splice variant or truncated form of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition. Nucleic acids can be truncated at the 5′ end or at the 3′ end. Polypeptides can be truncated at the N-terminal end or the C-terminal end. Truncated versions of nucleic acid or polypeptide sequences can be naturally occurring or created using recombinant techniques.

The terms “genetic variant” and “nucleotide variant” are used herein interchangeably to refer to changes or alterations to the reference human gene or cDNA sequence at a particular locus, including, but not limited to, nucleotide base deletions, insertions, inversions, and substitutions in the coding and non-coding regions. Deletions may be of a single nucleotide base, a portion or a region of the nucleotide sequence of the gene, or of the entire gene sequence. Insertions may be of one or more nucleotide bases. The genetic variant or nucleotide variant may occur in transcriptional regulatory regions, untranslated regions of mRNA, exons, introns, exon/intron junctions, etc. The genetic variant or nucleotide variant can potentially result in stop codons, frame shifts, deletions of amino acids, altered gene transcript splice forms or altered amino acid sequence.

An allele or gene allele comprises generally a naturally occurring gene having a reference sequence or a gene containing a specific nucleotide variant.

A haplotype refers to a combination of genetic (nucleotide) variants in a region of an mRNA or a genomic DNA on a chromosome found in an individual. Thus, a haplotype includes a number of genetically linked polymorphic variants which are typically inherited together as a unit.

As used herein, the term “amino acid variant” is used to refer to an amino acid change to a reference human protein sequence resulting from genetic variants or nucleotide variants to the reference human gene encoding the reference protein. The term “amino acid variant” is intended to encompass not only single amino acid substitutions, but also amino acid deletions, insertions, and other significant changes of amino acid sequence in the reference protein.

The term “genotype” as used herein means the nucleotide characters at a particular nucleotide variant marker (or locus) in either one allele or both alleles of a gene (or a particular chromosome region). With respect to a particular nucleotide position of a gene of interest, the nucleotide(s) at that locus or equivalent thereof in one or both alleles form the genotype of the gene at that locus. A genotype can be homozygous or heterozygous. Accordingly, “genotyping” means determining the genotype, that is, the nucleotide(s) at a particular gene locus. Genotyping can also be done by determining the amino acid variant at a particular position of a protein which can be used to deduce the corresponding nucleotide variant(s).

The term “locus” refers to a specific position or site in a gene sequence or protein. Thus, there may be one or more contiguous nucleotides in a particular gene locus, or one or more amino acids at a particular locus in a polypeptide. Moreover, a locus may refer to a particular position in a gene where one or more nucleotides have been deleted, inserted, or inverted.

Unless specified otherwise or understood by one of skill in art, the terms “polypeptide,” “protein,” and “peptide” are used interchangeably herein to refer to an amino acid chain in which the amino acid residues are linked by covalent peptide bonds. The amino acid chain can be of any length of at least two amino acids, including full-length proteins. Unless otherwise specified, polypeptide, protein, and peptide also encompass various modified forms thereof, including but not limited to glycosylated forms, phosphorylated forms, etc. A polypeptide, protein or peptide can also be referred to as a gene product.

Lists of gene and gene products that can be assayed by molecular profiling techniques are presented herein. Lists of genes may be presented in the context of molecular profiling techniques that detect a gene product (e.g., an mRNA or protein). One of skill will understand that this implies detection of the gene product of the listed genes. Similarly, lists of gene products may be presented in the context of molecular profiling techniques that detect a gene sequence or copy number. One of skill will understand that this implies detection of the gene corresponding to the gene products, including as an example DNA encoding the gene products. As will be appreciated by those skilled in the art, a “biomarker” or “marker” comprises a gene and/or gene product depending on the context.

The terms “label” and “detectable label” can refer to any composition detectable by spectroscopic, photochemical, biochemical, immunochemical, electrical, optical, chemical or similar methods. Such labels include biotin for staining with labeled streptavidin conjugate, magnetic beads (e.g., DYNABEADS™), fluorescent dyes (e.g., fluorescein, Texas red, rhodamine, green fluorescent protein, and the like), radiolabels (e.g., ³H, ¹²¹, ³⁵S, ¹⁴C, or ³²P), enzymes (e.g., horse radish peroxidase, alkaline phosphatase and others commonly used in an ELISA), and calorimetric labels such as colloidal gold or colored glass or plastic (e.g., polystyrene, polypropylene, latex, etc) beads. Patents teaching the use of such labels include U.S. Pat. Nos. 3,817,837; 3,850,752; 3,939,350; 3,996,345; 4,277,437; 4,275,149; and 4,366,241. Means of detecting such labels are well known to those of skill in the art. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and calorimetric labels are detected by simply visualizing the colored label. Labels can include, e.g., ligands that bind to labeled antibodies, fluorophores, chemiluminescent agents, enzymes, and antibodies which can serve as specific binding pair members for a labeled ligand. An introduction to labels, labeling procedures and detection of labels is found in Polak and Van Noorden Introduction to Immunocytochemistry, 2nd ed., Springer Verlag, N Y (1997); and in Haugland Handbook of Fluorescent Probes and Research Chemicals, a combined handbook and catalogue Published by Molecular Probes, Inc. (1996).

Detectable labels include, but are not limited to, nucleotides (labeled or unlabelled), compomers, sugars, peptides, proteins, antibodies, chemical compounds, conducting polymers, binding moieties such as biotin, mass tags, calorimetric agents, light emitting agents, chemiluminescent agents, light scattering agents, fluorescent tags, radioactive tags, charge tags (electrical or magnetic charge), volatile tags and hydrophobic tags, biomolecules (e.g., members of a binding pair antibody/antigen, antibody/antibody, antibody/antibody fragment, antibody/antibody receptor, antibody/protein A or protein G, hapten/anti-hapten, biotin/avidin, biotin/streptavidin, folic acid/folate binding protein, vitamin B12/intrinsic factor, chemical reactive group/complementary chemical reactive group (e.g., sulfhydryl/maleimide, sulfhydryl/haloacetyl derivative, amine/isotriocyanate, amine/succinimidyl ester, and amine/sulfonyl halides) and the like.

The term “antibody” as used herein encompasses naturally occurring antibodies as well as non-naturally occurring antibodies, including, for example, single chain antibodies, chimeric, bifunctional and humanized antibodies, as well as antigen-binding fragments thereof, (e.g., Fab′, F(ab′)₂, Fab, Fv and rIgG). See also, Pierce Catalog and Handbook, 1994-1995 (Pierce Chemical Co., Rockford, Ill.). See also, e.g., Kuby, J., Immunology, 3.sup.rd Ed., W. H. Freeman & Co., New York (1998). Such non-naturally occurring antibodies can be constructed using solid phase peptide synthesis, can be produced recombinantly or can be obtained, for example, by screening combinatorial libraries consisting of variable heavy chains and variable light chains as described by Huse et al., Science 246:1275-1281 (1989), which is incorporated herein by reference. These and other methods of making, for example, chimeric, humanized, CDR-grafted, single chain, and bifunctional antibodies are well known to those skilled in the art. See, e.g., Winter and Harris, Immunol. Today 14:243-246 (1993); Ward et al., Nature 341:544-546 (1989); Harlow and Lane, Antibodies, 511-52, Cold Spring Harbor Laboratory publications, New York, 1988; Hilyard et al., Protein Engineering: A practical approach (IRL Press 1992); Borrebaeck, Antibody Engineering, 2d ed. (Oxford University Press 1995); each of which is incorporated herein by reference.

Unless otherwise specified, antibodies can include both polyclonal and monoclonal antibodies. Antibodies also include genetically engineered forms such as chimeric antibodies (e.g., humanized murine antibodies) and heteroconjugate antibodies (e.g., bispecific antibodies). The term also refers to recombinant single chain Fv fragments (scFv). The term antibody also includes bivalent or bispecific molecules, diabodies, triabodies, and tetrabodies. Bivalent and bispecific molecules are described in, e.g., Kostelny et al. (1992) J Immunol 148:1547, Pack and Pluckthun (1992) Biochemistry 31:1579, Holliger et al. (1993) Proc Natl Acad Sci USA. 90:6444, Gruber et al. (1994) J Immunol:5368, Zhu et al. (1997) Protein Sci 6:781, Hu et al. (1997) Cancer Res. 56:3055, Adams et al. (1993) Cancer Res. 53:4026, and McCartney, et al. (1995) Protein Eng. 8:301.

Typically, an antibody has a heavy and light chain. Each heavy and light chain contains a constant region and a variable region, (the regions are also known as “domains”). Light and heavy chain variable regions contain four framework regions interrupted by three hyper-variable regions, also called complementarity-determining regions (CDRs). The extent of the framework regions and CDRs have been defined. The sequences of the framework regions of different light or heavy chains are relatively conserved within a species. The framework region of an antibody, that is the combined framework regions of the constituent light and heavy chains, serves to position and align the CDRs in three dimensional spaces. The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found. References to V_{H} refer to the variable region of an immunoglobulin heavy chain of an antibody, including the heavy chain of an Fv, scFv, or Fab. References to V_{L} refer to the variable region of an immunoglobulin light chain, including the light chain of an Fv, scFv, dsFv or Fab.

The phrase “single chain Fv” or “scFv” refers to an antibody in which the variable domains of the heavy chain and of the light chain of a traditional two chain antibody have been joined to form one chain. Typically, a linker peptide is inserted between the two chains to allow for proper folding and creation of an active binding site. A “chimeric antibody” is an immunoglobulin molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

A “humanized antibody” is an immunoglobulin molecule that contains minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody optimally also will comprise at least a portion of an immunoglobulin constant region (Fc), typically that of a human immunoglobulin (Jones et al., Nature 321:522-525 (1986); Riechmann et al., Nature 332:323-327 (1988); and Presta, Curr. Op. Struct. Biol. 2:593-596 (1992)). Humanization can be essentially performed following the method of Winter and co-workers (Jones et al., Nature 321:522-525 (1986); Riechmann et al., Nature 332:323-327 (1988); Verhoeyen et al., Science 239:1534-1536 (1988)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species.

The terms “epitope” and “antigenic determinant” refer to a site on an antigen to which an antibody binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996).

The terms “primer”, “probe,” and “oligonucleotide” are used herein interchangeably to refer to a relatively short nucleic acid fragment or sequence. They can comprise DNA, RNA, or a hybrid thereof, or chemically modified analog or derivatives thereof. Typically, they are single-stranded. However, they can also be double-stranded having two complementing strands which can be separated by denaturation. Normally, primers, probes and oligonucleotides have a length of from about 8 nucleotides to about 200 nucleotides, preferably from about 12 nucleotides to about 100 nucleotides, and more preferably about 18 to about 50 nucleotides. They can be labeled with detectable markers or modified using conventional manners for various molecular biological applications.

The term “isolated” when used in reference to nucleic acids (e.g., genomic DNAs, cDNAs, mRNAs, or fragments thereof) is intended to mean that a nucleic acid molecule is present in a form that is substantially separated from other naturally occurring nucleic acids that are normally associated with the molecule. Because a naturally existing chromosome (or a viral equivalent thereof) includes a long nucleic acid sequence, an isolated nucleic acid can be a nucleic acid molecule having only a portion of the nucleic acid sequence in the chromosome but not one or more other portions present on the same chromosome. More specifically, an isolated nucleic acid can include naturally occurring nucleic acid sequences that flank the nucleic acid in the naturally existing chromosome (or a viral equivalent thereof). An isolated nucleic acid can be substantially separated from other naturally occurring nucleic acids that are on a different chromosome of the same organism. An isolated nucleic acid can also be a composition in which the specified nucleic acid molecule is significantly enriched so as to constitute at least 10%, 20%, 30%, 40%, 50%, 60%, 70%, 80%, 90%, 95%, or at least 99% of the total nucleic acids in the composition.

An isolated nucleic acid can be a hybrid nucleic acid having the specified nucleic acid molecule covalently linked to one or more nucleic acid molecules that are not the nucleic acids naturally flanking the specified nucleic acid. For example, an isolated nucleic acid can be in a vector. In addition, the specified nucleic acid may have a nucleotide sequence that is identical to a naturally occurring nucleic acid or a modified form or mutein thereof having one or more mutations such as nucleotide substitution, deletion/insertion, inversion, and the like.

An isolated nucleic acid can be prepared from a recombinant host cell (in which the nucleic acids have been recombinantly amplified and/or expressed), or can be a chemically synthesized nucleic acid having a naturally occurring nucleotide sequence or an artificially modified form thereof.

The term “isolated polypeptide” as used herein is defined as a polypeptide molecule that is present in a form other than that found in nature. Thus, an isolated polypeptide can be a non-naturally occurring polypeptide. For example, an isolated polypeptide can be a “hybrid polypeptide.” An isolated polypeptide can also be a polypeptide derived from a naturally occurring polypeptide by additions or deletions or substitutions of amino acids. An isolated polypeptide can also be a “purified polypeptide” which is used herein to mean a composition or preparation in which the specified polypeptide molecule is significantly enriched so as to constitute at least 10% of the total protein content in the composition. A “purified polypeptide” can be obtained from natural or recombinant host cells by standard purification techniques, or by chemically synthesis, as will be apparent to skilled artisans.

The terms “hybrid protein,” “hybrid polypeptide,” “hybrid peptide,” “fusion protein,” “fusion polypeptide,” and “fusion peptide” are used herein interchangeably to mean a non-naturally occurring polypeptide or isolated polypeptide having a specified polypeptide molecule covalently linked to one or more other polypeptide molecules that do not link to the specified polypeptide in nature. Thus, a “hybrid protein” may be two naturally occurring proteins or fragments thereof linked together by a covalent linkage. A “hybrid protein” may also be a protein formed by covalently linking two artificial polypeptides together. Typically but not necessarily, the two or more polypeptide molecules are linked or “fused” together by a peptide bond forming a single non-branched polypeptide chain.

The term “high stringency hybridization conditions,” when used in connection with nucleic acid hybridization, includes hybridization conducted overnight at 42° C. in a solution containing 50% formamide, 5×SSC (750 mM NaCl, 75 mM sodium citrate), 50 mM sodium phosphate, pH 7.6, 5×Denhardt's solution, 10% dextran sulfate, and 20 microgram/ml denatured and sheared salmon sperm DNA, with hybridization filters washed in 0.1×SSC at about 65° C. The term “moderate stringent hybridization conditions,” when used in connection with nucleic acid hybridization, includes hybridization conducted overnight at 37° C. in a solution containing 50% formamide, 5×SSC (750 mM NaCl, 75 mM sodium citrate), 50 mM sodium phosphate, pH 7.6, 5×Denhardt's solution, 10% dextran sulfate, and 20 microgram/ml denatured and sheared salmon sperm DNA, with hybridization filters washed in 1×SSC at about 50° C. It is noted that many other hybridization methods, solutions and temperatures can be used to achieve comparable stringent hybridization conditions as will be apparent to skilled artisans.

For the purpose of comparing two different nucleic acid or polypeptide sequences, one sequence (test sequence) may be described to be a specific percentage identical to another sequence (comparison sequence). The percentage identity can be determined by the algorithm of Karlin and Altschul, Proc. Natl. Acad. Sci. USA, 90:5873-5877 (1993), which is incorporated into various BLAST programs. The percentage identity can be determined by the “BLAST 2 Sequences” tool, which is available at the National Center for Biotechnology Information (NCBI) website. See Tatusova and Madden, FEMS Microbiol. Lett., 174(2):247-250 (1999). For pairwise DNA-DNA comparison, the BLASTN program is used with default parameters (e.g., Match: 1; Mismatch: −2; Open gap: 5 penalties; extension gap: 2 penalties; gap x_dropoff: 50; expect: 10; and word size: 11, with filter). For pairwise protein-protein sequence comparison, the BLASTP program can be employed using default parameters (e.g., Matrix: BLOSUM62; gap open: 11; gap extension: 1; x_dropoff: 15; expect: 10.0; and wordsize: 3, with filter). Percent identity of two sequences is calculated by aligning a test sequence with a comparison sequence using BLAST, determining the number of amino acids or nucleotides in the aligned test sequence that are identical to amino acids or nucleotides in the same position of the comparison sequence, and dividing the number of identical amino acids or nucleotides by the number of amino acids or nucleotides in the comparison sequence. When BLAST is used to compare two sequences, it aligns the sequences and yields the percent identity over defined, aligned regions. If the two sequences are aligned across their entire length, the percent identity yielded by the BLAST is the percent identity of the two sequences. If BLAST does not align the two sequences over their entire length, then the number of identical amino acids or nucleotides in the unaligned regions of the test sequence and comparison sequence is considered to be zero and the percent identity is calculated by adding the number of identical amino acids or nucleotides in the aligned regions and dividing that number by the length of the comparison sequence. Various versions of the BLAST programs can be used to compare sequences, e.g., BLAST 2.1.2 or BLAST+2.2.22.

A subject or individual can be any animal which may benefit from the methods of the invention, including, e.g., humans and non-human mammals, such as primates, rodents, horses, dogs and cats. Subjects include without limitation a eukaryotic organisms, most preferably a mammal such as a primate, e.g., chimpanzee or human, cow; dog; cat; a rodent, e.g., guinea pig, rat, mouse; rabbit; or a bird; reptile; or fish. Subjects specifically intended for treatment using the methods described herein include humans. A subject may be referred to as an individual or a patient.

Treatment of a disease or individual according to the invention is an approach for obtaining beneficial or desired medical results, including clinical results, but not necessarily a cure. For purposes of this invention, beneficial or desired clinical results include, but are not limited to, alleviation or amelioration of one or more symptoms, diminishment of extent of disease, stabilized (i.e., not worsening) state of disease, preventing spread of disease, delay or slowing of disease progression, amelioration or palliation of the disease state, and remission (whether partial or total), whether detectable or undetectable. Treatment also includes prolonging survival as compared to expected survival if not receiving treatment or if receiving a different treatment. A treatment can include administration of a therapeutic agent, which can be an agent that exerts a cytotoxic, cytostatic, or immunomodulatory effect on diseased cells, e.g., cancer cells, or other cells that may promote a diseased state, e.g., activated immune cells. Therapeutic agents selected by the methods of the invention are not limited. Any therapeutic agent can be selected where a link can be made between molecular profiling and potential efficacy of the agent. Therapeutic agents include without limitation drugs, pharmaceuticals, small molecules, protein therapies, antibody therapies, viral therapies, gene therapies, and the like. Cancer treatments or therapies include apoptosis-mediated and non-apoptosis mediated cancer therapies including, without limitation, chemotherapy, hormonal therapy, radiotherapy, immunotherapy, and combinations thereof. Chemotherapeutic agents comprise therapeutic agents and combinations of therapeutic agents that treat, cancer cells, e.g., by killing those cells. Examples of different types of chemotherapeutic drugs include without limitation alkylating agents (e.g., nitrogen mustard derivatives, ethylenimines, alkylsulfonates, hydrazines and triazines, nitrosureas, and metal salts), plant alkaloids (e.g., vinca alkaloids, taxanes, podophyllotoxins, and camptothecan analogs), antitumor antibiotics (e.g., anthracyclines, chromomycins, and the like), antimetabolites (e.g., folic acid antagonists, pyrimidine antagonists, purine antagonists, and adenosine deaminase inhibitors), topoisomerase I inhibitors, topoisomerase II inhibitors, and miscellaneous antineoplastics (e.g., ribonucleotide reductase inhibitors, adrenocortical steroid inhibitors, enzymes, antimicrotubule agents, and retinoids).

A biomarker refers generally to a molecule, including without limitation a gene or product thereof, nucleic acids (e.g., DNA, RNA), protein/peptide/polypeptide, carbohydrate structure, lipid, glycolipid, characteristics of which can be detected in a tissue or cell to provide information that is predictive, diagnostic, prognostic and/or theranostic for sensitivity or resistance to candidate treatment.

### Biological Samples

A sample as used herein includes any relevant biological sample that can be used for molecular profiling, e.g., sections of tissues such as biopsy or tissue removed during surgical or other procedures, bodily fluids, autopsy samples, and frozen sections taken for histological purposes. Such samples include blood and blood fractions or products (e.g., serum, buffy coat, plasma, platelets, red blood cells, and the like), sputum, malignant effusion, cheek cells tissue, cultured cells (e.g., primary cultures, explants, and transformed cells), stool, urine, other biological or bodily fluids (e.g., prostatic fluid, gastric fluid, intestinal fluid, renal fluid, lung fluid, cerebrospinal fluid, and the like), etc. The sample can comprise biological material that is a fresh frozen & formalin fixed paraffin embedded (FFPE) block, formalin-fixed paraffin embedded, or is within an RNA preservative+formalin fixative. More than one sample of more than one type can be used for each patient. In a preferred embodiment, the sample comprises a fixed tumor sample.

The sample used in the methods described herein can be a formalin fixed paraffin embedded (FFPE) sample. The FFPE sample can be one or more of fixed tissue, unstained slides, bone marrow core or clot, core needle biopsy, malignant fluids and fine needle aspirate (FNA). In an embodiment, the fixed tissue comprises a tumor containing formalin fixed paraffin embedded (FFPE) block from a surgery or biopsy. In another embodiment, the unstained slides comprise unstained, charged, unbaked slides from a paraffin block. In another embodiment, bone marrow core or clot comprises a decalcified core. A formalin fixed core and/or clot can be paraffin-embedded. In still another embodiment, the core needle biopsy comprises 1, 2, 3, 4, 5, 6, 7, 8, 9, 10 or more, e.g., 3-4, paraffin embedded biopsy samples. An 18 gauge needle biopsy can be used. The malignant fluid can comprise a sufficient volume of fresh pleural/ascitic fluid to produce a 5×5×2 mm cell pellet. The fluid can be formalin fixed in a paraffin block. In an embodiment, the core needle biopsy comprises 1, 2, 3, 4, 5, 6, 7, 8, 9, 10 or more, e.g., 4-6, paraffin embedded aspirates.

A sample may be processed according to techniques understood by those in the art. A sample can be without limitation fresh, frozen or fixed cells or tissue. In some embodiments, a sample comprises formalin-fixed paraffin-embedded (FFPE) tissue, fresh tissue or fresh frozen (FF) tissue. A sample can comprise cultured cells, including primary or immortalized cell lines derived from a subject sample. A sample can also refer to an extract from a sample from a subject. For example, a sample can comprise DNA, RNA or protein extracted from a tissue or a bodily fluid. Many techniques and commercial kits are available for such purposes. The fresh sample from the individual can be treated with an agent to preserve RNA prior to further processing, e.g., cell lysis and extraction. Samples can include frozen samples collected for other purposes. Samples can be associated with relevant information such as age, gender, and clinical symptoms present in the subject; source of the sample; and methods of collection and storage of the sample. A sample is typically obtained from a subject.

A biopsy comprises the process of removing a tissue sample for diagnostic or prognostic evaluation, and to the tissue specimen itself. Any biopsy technique known in the art can be applied to the molecular profiling methods of the present invention. The biopsy technique applied can depend on the tissue type to be evaluated (e.g., colon, prostate, kidney, bladder, lymph node, liver, bone marrow, blood cell, lung, breast, etc.), the size and type of the tumor (e.g., solid or suspended, blood or ascites), among other factors. Representative biopsy techniques include, but are not limited to, excisional biopsy, incisional biopsy, needle biopsy, surgical biopsy, and bone marrow biopsy. An “excisional biopsy” refers to the removal of an entire tumor mass with a small margin of normal tissue surrounding it. An “incisional biopsy” refers to the removal of a wedge of tissue that includes a cross-sectional diameter of the tumor. Molecular profiling can use a “core-needle biopsy” of the tumor mass, or a “fine-needle aspiration biopsy” which generally obtains a suspension of cells from within the tumor mass. Biopsy techniques are discussed, for example, in Harrison's Principles of Internal Medicine, Kasper, et al., eds., 16th ed., 2005, Chapter 70, and throughout Part V.

Standard molecular biology techniques known in the art and not specifically described are generally followed as in Sambrook et al., Molecular Cloning: A Laboratory Manual, Cold Spring Harbor Laboratory Press, New York (1989), and as in Ausubel et al., Current Protocols in Molecular Biology, John Wiley and Sons, Baltimore, Md. (1989) and as in Perbal, A Practical Guide to Molecular Cloning, John Wiley & Sons, New York (1988), and as in Watson et al., Recombinant DNA, Scientific American Books, New York and in Birren et al (eds) Genome Analysis: A Laboratory Manual Series, Vols. 1-4 Cold Spring Harbor Laboratory Press, New York (1998) and methodology as set forth in U.S. Pat. Nos. 4,666,828; 4,683,202; 4,801,531; 5,192,659 and 5,272,057 and incorporated herein by reference. Polymerase chain reaction (PCR) can be carried out generally as in PCR Protocols: A Guide to Methods and Applications, Academic Press, San Diego, Calif (1990).

### Vesicles

The sample can comprise vesicles. Methods of the invention can include assessing one or more vesicles, including assessing vesicle populations. A vesicle, as used herein, is a membrane vesicle that is shed from cells. Vesicles or membrane vesicles include without limitation: circulating microvesicles (cMVs), microvesicle, exosome, nanovesile, dexosome, bleb, blebby, prostasome, microparticle, intralumenal vesicle, membrane fragment, intralumnenal endosomal vesicle, endosomal-like vesicle, exocytosis vehicle, endosome vesicle, endosomal vesicle, apoptotic body, multivesicular body, secretory vesicle, phospholipid vesicle, liposomal vesicle, argosome, texasome, secresome, tolerosome, melanosome, oncosome, or exocytosed vehicle. Furthermore, although vesicles may be produced by different cellular processes, the methods of the invention are not limited to or reliant on any one mechanism, insofar as such vesicles are present in a biological sample and are capable of being characterized by the methods disclosed herein. Unless otherwise specified, methods that make use of a species of vesicle can be applied to other types of vesicles. Vesicles comprise spherical structures with a lipid bilayer similar to cell membranes which surrounds an inner compartment which can contain soluble components, sometimes referred to as the payload. In some embodiments, the methods of the invention make use of exosomes, which are small secreted vesicles of about 40-100 nm in diameter. For a review of membrane vesicles, including types and characterizations, see Thery et aH., Nat Rev Immunol. 2009 Aug; 9(8): 581-93. Some properties of different types of vesicles include those in Table 1:

TABLE 1
Vesicle Properties

| | | | | | Exosome- | |
|---|---|---|---|---|---|---|
| | | | | Membrane | like | Apoptotic |
| Feature | Exosomes | Microvesicles | Ectosomes | particles | vesicles | vesicles |
| Size | 50-100 nm | 100-1,000 nm | 50-200 nm | 50-80 nm | 20-50 nm | 50-500 nm |
| Density in | 1.13-1.19 g/ml | | | 1.04-1.07 g/ml | 1.1 g/ml | 1.16-1.28 g/ml |
| sucrose | | | | | | |
| EM | Cup shape | Irregular | Bilamellar | Round | Irregular | Heterogeneous |
| appearance | | shape, | round | | shape | |
| | | electron | structures | | | |
| | | dense | | | | |
| Sedimentation | 100,000 g | 10,000 g | 160,000- | 100,000- | 175,000 g | 1,200 g, |
| | | | 200,000 g | 200,000 g | | 10,000 g, |
| | | | | | | 100,000 g |
| Lipid | Enriched in | Expose PPS | Enriched in | | No lipid | |
| composition | cholesterol, | | cholesterol | | rafts | |
| | sphingomyelin | | and | | | |
| | and ceramide; | | diacylglycerol; | | | |
| | contains lipid | | expose PPS | | | |
| | rafts; expose | | | | | |
| | PPS | | | | | |
| Major | Tetraspanins | Integrins, | CR1 and | CD133; | TNFRI | Histones |
| protein | (e.g., CD63, | selectins and | proteolytic | no CD63 | | |
| markers | CD9), Alix, | CD40 ligand | enzymes; | | | |
| | TSG101 | | no CD63 | | | |
| Intracellular | Internal | Plasma | Plasma | Plasma | | |
| origin | compartments | membrane | membrane | membrane | | |
| | (endosomes) | | | | | |

Abbreviations:
phosphatidylserine (PPS);
electron microscopy (EM)

Vesicles include shed membrane bound particles, or “microparticles,” that are derived from either the plasma membrane or an internal membrane. Vesicles can be released into the extracellular environment from cells. Cells releasing vesicles include without limitation cells that originate from, or are derived from, the ectoderm, endoderm, or mesoderm. The cells may have undergone genetic, environmental, and/or any other variations or alterations. For example, the cell can be tumor cells. A vesicle can reflect any changes in the source cell, and thereby reflect changes in the originating cells, e.g., cells having various genetic mutations. In one mechanism, a vesicle is generated intracellularly when a segment of the cell membrane spontaneously invaginates and is ultimately exocytosed (see for example, Keller et al., Immunol. Lett. 107 (2): 102-8 (2006)). Vesicles also include cell-derived structures bounded by a lipid bilayer membrane arising from both herniated evagination (blebbing) separation and sealing of portions of the plasma membrane or from the export of any intracellular membrane-bounded vesicular structure containing various membrane-associated proteins of tumor origin, including surface-bound molecules derived from the host circulation that bind selectively to the tumor-derived proteins together with molecules contained in the vesicle lumen, including but not limited to tumor-derived microRNAs or intracellular proteins. Blebs and blebbing are further described in Charras et al., Nature Reviews Molecular and Cell Biology, Vol. 9, No. 11, p. 730-736 (2008). A vesicle shed into circulation or bodily fluids from tumor cells may be referred to as a “circulating tumor-derived vesicle.” When such vesicle is an exosome, it may be referred to as a circulating-tumor derived exosome (CTE). In some instances, a vesicle can be derived from a specific cell of origin. CTE, as with a cell-of-origin specific vesicle, typically have one or more unique biomarkers that permit isolation of the CTE or cell-of-origin specific vesicle, e.g., from a bodily fluid and sometimes in a specific manner. For example, a cell or tissue specific markers are used to identify the cell of origin. Examples of such cell or tissue specific markers are disclosed herein and can further be accessed in the Tissue-specific Gene Expression and Regulation (TiGER) Database, available at bioinfo.wilmer.jhu.edu/tiger/; Liu et al. (2008) TiGER: a database for tissue-specific gene expression and regulation. BMC Bioinformatics. 9:271; TissueDistributionDBs, available at genome.dkfz-heidelberg.de/menu/tissue_db/index.html.

A vesicle can have a diameter of greater than about 10 nm, 20 nm, or 30 n. A vesicle can have a diameter of greater than 40 n, 50 nm, 100 nm, 200 nm, 500 nm, 1000 nm or greater than 10,000 nm. A vesicle can have a diameter of about 30-1000 nm, about 30-800 nm, about 30-200 n, or about 30-100 nm. In some embodiments, the vesicle has a diameter of less than 10,000 nm, 1000 nm, 800 nm, 500 nm, 200 nm, 100 nm, 50 nm, 40 nm, 30 nm, 20 nm or less than 10 nm. As used herein the term “about” in reference to a numerical value means that variations of 10% above or below the numerical value are within the range ascribed to the specified value. Typical sizes for various types of vesicles are shown in Table 1. Vesicles can be assessed to measure the diameter of a single vesicle or any number of vesicles. For example, the range of diameters of a vesicle population or an average diameter of a vesicle population can be determined. Vesicle diameter can be assessed using methods known in the art, e.g., imaging technologies such as electron microscopy. In an embodiment, a diameter of one or more vesicles is determined using optical particle detection. See, e.g., U.S. Pat. No. 7,751,053, entitled “Optical Detection and Analysis of Particles” and issued Jul. 6, 2010; and U.S. Pat. No. 7,399,600, entitled “Optical Detection and Analysis of Particles” and issued Jul. 15, 2010.

In some embodiments, vesicles are directly assayed from a biological sample without prior isolation, purification, or concentration from the biological sample. For example, the amount of vesicles in the sample can by itself provide a biosignature that provides a diagnostic, prognostic or theranostic determination. Alternatively, the vesicle in the sample may be isolated, captured, purified, or concentrated from a sample prior to analysis. As noted, isolation, capture or purification as used herein comprises partial isolation, partial capture or partial purification apart from other components in the sample. Vesicle isolation can be performed using various techniques as described herein or known in the art, including without limitation size exclusion chromatography, density gradient centrifugation, differential centrifugation, nanomembrane ultrafiltration, immunoabsorbent capture, affinity purification, affinity capture, immunoassay, immunoprecipitation, microfluidic separation, flow cytometry or combinations thereof.

Vesicles can be assessed to provide a phenotypic characterization by comparing vesicle characteristics to a reference. In some embodiments, surface antigens on a vesicle are assessed. A vesicle or vesicle population carrying a specific marker can be referred to as a positive (biomarker+) vesicle or vesicle population. For example, a DLL4+population refers to a vesicle population associated with DLL4. Conversely, a DLL4-population would not be associated with DLL4. The surface antigens can provide an indication of the anatomical origin and/or cellular of the vesicles and other phenotypic information, e.g., tumor status. For example, vesicles found in a patient sample can be assessed for surface antigens indicative of colorectal origin and the presence of cancer, thereby identifying vesicles associated with colorectal cancer cells. The surface antigens may comprise any informative biological entity that can be detected on the vesicle membrane surface, including without limitation surface proteins, lipids, carbohydrates, and other membrane components. For example, positive detection of colon derived vesicles expressing tumor antigens can indicate that the patient has colorectal cancer. As such, methods of the invention can be used to characterize any disease or condition associated with an anatomical or cellular origin, by assessing, for example, disease-specific and cell-specific biomarkers of one or more vesicles obtained from a subject.

In embodiments, one or more vesicle payloads are assessed to provide a phenotypic characterization. The payload with a vesicle comprises any informative biological entity that can be detected as encapsulated within the vesicle, including without limitation proteins and nucleic acids, e.g., genomic or cDNA, mRNA, or functional fragments thereof, as well as microRNAs (miRs). In addition, methods of the invention are directed to detecting vesicle surface antigens (in addition or exclusive to vesicle payload) to provide a phenotypic characterization. For example, vesicles can be characterized by using binding agents (e.g., antibodies or aptamers) that are specific to vesicle surface antigens, and the bound vesicles can be further assessed to identify one or more payload components disclosed therein. As described herein, the levels of vesicles with surface antigens of interest or with payload of interest can be compared to a reference to characterize a phenotype. For example, overexpression in a sample of cancer-related surface antigens or vesicle payload, e.g., a tumor associated mRNA or microRNA, as compared to a reference, can indicate the presence of cancer in the sample. The biomarkers assessed can be present or absent, increased or reduced based on the selection of the desired target sample and comparison of the target sample to the desired reference sample. Non-limiting examples of target samples include: disease; treated/not-treated; different time points, such as a in a longitudinal study; and non-limiting examples of reference sample: non-disease; normal; different time points; and sensitive or resistant to candidate treatment(s).

In an embodiment, molecular profiling of the invention comprises analysis of microvesicles, such as circulating microvesicles.

### MicroRNA

Various biomarker molecules can be assessed in biological samples or vesicles obtained from such biological samples. MicroRNAs comprise one class biomarkers assessed via methods of the invention. MicroRNAs, also referred to herein as miRNAs or miRs, are short RNA strands approximately 21-23 nucleotides in length. MiRNAs are encoded by genes that are transcribed from DNA but are not translated into protein and thus comprise non-coding RNA. The miRs are processed from primary transcripts known as pri-miRNA to short stem-loop structures called pre-miRNA and finally to the resulting single strand miRNA. The pre-miRNA typically forms a structure that folds back on itself in self-complementary regions. These structures are then processed by the nuclease Dicer in animals or DCL1 in plants. Mature miRNA molecules are partially complementary to one or more messenger RNA (mRNA) molecules and can function to regulate translation of proteins. Identified sequences of miRNA can be accessed at publicly available databases.

miRNAs are generally assigned a number according to the naming convention “mir-[number].” The number of a miRNA is assigned according to its order of discovery relative to previously identified miRNA species. For example, if the last published miRNA was mir-121, the next discovered miRNA will be named mir-122, etc. When a miRNA is discovered that is homologous to a known miRNA from a different organism, the name can be given an optional organism identifier, of the form [organism identifier]-mir-[number]. Identifiers include hsa for Homo sapiens and mmu for Mus Musculus. For example, a human homolog to mir-121 might be referred to as hsa-mir-121 whereas the mouse homolog can be referred to as mmu-mir-121.

Mature microRNA is commonly designated with the prefix “miR” whereas the gene or precursor miRNA is designated with the prefix “mir.” For example, mir-121 is a precursor for miR-121. When differing miRNA genes or precursors are processed into identical mature miRNAs, the genes/precursors can be delineated by a numbered suffix. For example, mir-121-1 and mir-121-2 can refer to distinct genes or precursors that are processed into miR-121. Lettered suffixes are used to indicate closely related mature sequences. For example, mir-121a and mir-121b can be processed to closely related miRNAs miR-121a and miR-121b, respectively. In the context of the invention, any microRNA (miRNA or miR) designated herein with the prefix mir-* or miR-* is understood to encompass both the precursor and/or mature species, unless otherwise explicitly stated otherwise.

Sometimes it is observed that two mature miRNA sequences originate from the same precursor. When one of the sequences is more abundant that the other, a “*” suffix can be used to designate the less common variant. For example, miR-121 would be the predominant product whereas miR-121* is the less common variant found on the opposite arm of the precursor. If the predominant variant is not identified, the miRs can be distinguished by the suffix “5p” for the variant from the 5′ arm of the precursor and the suffix “3p” for the variant from the 3′ arm. For example, miR-121-5p originates from the 5′ arm of the precursor whereas miR-121-3p originates from the 3′ arm. Less commonly, the 5p and 3p variants are referred to as the sense (“s”) and anti-sense (“as”) forms, respectively. For example, miR-121-5p may be referred to as miR-121-s whereas miR-121-3p may be referred to as miR-121-as.

The above naming conventions have evolved over time and are general guidelines rather than absolute rules. For example, the let- and lin-families of miRNAs continue to be referred to by these monikers. The mir/miR convention for precursor/mature forms is also a guideline and context should be taken into account to determine which form is referred to. Further details of miR naming can be found at, e. g., Ambros et al. A uniform system for microRNA annotation. RNA 9:277-279 (2003).

Plant miRNAs follow a different naming convention as described in Meyers et al., Plant Cell. 2008 20(12):3186-3190.

A number of miRNAs are involved in gene regulation, and miRNAs are part of a growing class of non-coding RNAs that is now recognized as a major tier of gene control. In some cases, miRNAs can interrupt translation by binding to regulatory sites embedded in the 3′-UTRs of their target mRNAs, leading to the repression of translation. Target recognition involves complementary base pairing of the target site with the miRNA's seed region (positions 2-8 at the miRNA's 5′ end), although the exact extent of seed complementarity is not precisely determined and can be modified by 3′ pairing. In other cases, miRNAs function like small interfering RNAs (siRNA) and bind to perfectly complementary mRNA sequences to destroy the target transcript.

Characterization of a number of miRNAs indicates that they influence a variety of processes, including early development, cell proliferation and cell death, apoptosis and fat metabolism. For example, some miRNAs, such as lin-4, let-7, mir-14, mir-23, and bantam, have been shown to play critical roles in cell differentiation and tissue development. Others are believed to have similarly important roles because of their differential spatial and temporal expression patterns.

The miRNA database available from miRBase comprises a searchable database of published miRNA sequences and annotation. Further information about miRBase can be found in the following articles, each of which is incorporated by reference in its entirety herein: Griffiths-Jones et al., miRBase: tools for microRNA genomics. NAR 2008 36(Database Issue):D154-D158: Griffiths-ones et at miRBase: microRNA sequences, targets and gene nomenclature. NAR 2006 34(Database Issue):D140-D144; and Griffiths-Jones. S. The microRNA Registry NAR 2004 32(Database issue):D109-D111. Representative miRNAs contained in Release 16 of miRBase, made available September 2010.

As described herein, microRNAs are known to be involved in cancer and other diseases and can be assessed in order to characterize a phenotype in a sample. See, e.g., Ferracin et al., Micromarkers: miRNAs in cancer diagnosis and prognosis, Exp Rev Mol Diag, Apr 2010, Vol. 10, No. 3, Pages 297-308; Fabbri, miRNAs as molecular biomarkers of cancer, Exp Rev Mol Diag, May 2010, Vol. 10, No. 4, Pages 435-444.

In an embodiment, molecular profiling of the invention comprises analysis of microRNA.

Techniques to isolate and characterize vesicles and miRs are known to those of skill in the art. In addition to the methodology presented herein, additional methods can be found in U.S. Pat. Nos. 7,888,035, entitled “METHODS FOR ASSESSING RNA PATTERNS” and issued Feb. 15, 2011; and U.S. Pat. No. 7,897,356, entitled “METHODS AND SYSTEMS OF USING EXOSOMES FOR DETERMINING PHENOTYPES” and issued Mar. 1, 2011; and International Patent Publication Nos. WO/2011/066589, entitled “METHODS AND SYSTEMS FOR ISOLATING, STORING, AND ANALYZING VESICLES” and filed Nov. 30, 2010; WO/2011/088226, entitled “DETECTION OF GASTROINTESTINAL DISORDERS” and filed Jan. 13, 2011; WO/2011/109440, entitled “BIOMARKERS FOR THERANOSTICS” and filed Mar. 1, 2011; and WO/2011/127219, entitled “CIRCULATING BIOMARKERS FOR DISEASE” and filed Apr. 6, 2011, each of which applications are incorporated by reference herein in their entirety.

### Circulating Biomarkers

Circulating biomarkers include biomarkers that are detectable in body fluids, such as blood, plasma, serum. Examples of circulating cancer biomarkers include cardiac troponin T (cTnT), prostate specific antigen (PSA) for prostate cancer and CA125 for ovarian cancer. Circulating biomarkers according to the invention include any appropriate biomarker that can be detected in bodily fluid, including without limitation protein, nucleic acids, e.g., DNA, mRNA and microRNA, lipids, carbohydrates and metabolites. Circulating biomarkers can include biomarkers that are not associated with cells, such as biomarkers that are membrane associated, embedded in membrane fragments, part of a biological complex, or free in solution. In one embodiment, circulating biomarkers are biomarkers that are associated with one or more vesicles present in the biological fluid of a subject.

Circulating biomarkers have been identified for use in characterization of various phenotypes, such as detection of a cancer. See, e.g., Ahmed N, et al., Proteomic-based identification of haptoglobin-1 precursor as a novel circulating biomarker of ovarian cancer. Br. J. Cancer 2004; Mathelin et al., Circulating proteinic biomarkers and breast cancer, Gynecol Obstet Fertil. 2006 Jul-Aug; 34(7-8):638-46. Epub 2006 Jul 28; Ye et al., Recent technical strategies to identify diagnostic biomarkers for ovarian cancer. Expert Rev Proteomics. 2007 February; 4(1):121-31; Carney, Circulating oncoproteins HER2/neu, EGFR and CAIX (MN) as novel cancer biomarkers. Expert Rev Mol Diagn. 2007 May; 7(3):309-19; Gagnon, Discovery and application of protein biomarkers for ovarian cancer, Curr Opin Obstet Gynecol. 2008 Feb; 20(1):9-13; Pasterkamp et al., Immune regulatory cells: circulating biomarker factories in cardiovascular disease. Clin Sci (Lond). 2008 August; 115(4):129-31; Fabbri, miRNAs as molecular biomarkers of cancer, Exp Rev Mol Diag, May 2010, Vol. 10, No. 4, Pages 435-444; PCT Patent Publication WO/2007/088537; U.S. Pat. Nos. 7,745,150 and 7,655,479; U.S. Patent Publications 20110008808, 20100330683, 20100248290, 20100222230, 20100203566, 20100173788, 20090291932, 20090239246, 20090226937, 20090111121, 20090004687, 20080261258, 20080213907, 20060003465, 20050124071, and 20040096915, each of which publication is incorporated herein by reference in its entirety. In an embodiment, molecular profiling of the invention comprises analysis of circulating biomarkers.

### Gene Expression Profiling

The methods and systems of the invention comprise expression profiling, which includes assessing differential expression of one or more target genes disclosed herein. Differential expression can include overexpression and/or underexpression of a biological product, e.g., a gene, mRNA or protein, compared to a control (or a reference). The control can include similar cells to the sample but without the disease (e.g., expression profiles obtained from samples from healthy individuals). A control can be a previously determined level that is indicative of a drug target efficacy associated with the particular disease and the particular drug target. The control can be derived from the same patient, e.g., a normal adjacent portion of the same organ as the diseased cells, the control can be derived from healthy tissues from other patients, or previously determined thresholds that are indicative of a disease responding or not-responding to a particular drug target. The control can also be a control found in the same sample, e.g. a housekeeping gene or a product thereof (e.g., mRNA or protein). For example, a control nucleic acid can be one which is known not to differ depending on the cancerous or non-cancerous state of the cell. The expression level of a control nucleic acid can be used to normalize signal levels in the test and reference populations. Illustrative control genes include, but are not limited to, e.g., β-actin, glyceraldehyde 3-phosphate dehydrogenase and ribosomal protein P1. Multiple controls or types of controls can be used. The source of differential expression can vary. For example, a gene copy number may be increased in a cell, thereby resulting in increased expression of the gene. Alternately, transcription of the gene may be modified, e.g., by chromatin remodeling, differential methylation, differential expression or activity of transcription factors, etc. Translation may also be modified, e.g., by differential expression of factors that degrade mRNA, translate mRNA, or silence translation, e.g., microRNAs or siRNAs. In some embodiments, differential expression comprises differential activity. For example, a protein may carry a mutation that increases the activity of the protein, such as constitutive activation, thereby contributing to a diseased state. Molecular profiling that reveals changes in activity can be used to guide treatment selection.

Methods of gene expression profiling include methods based on hybridization analysis of polynucleotides, and methods based on sequencing of polynucleotides. Commonly used methods known in the art for the quantification of mRNA expression in a sample include northern blotting and in situ hybridization (Parker & Barnes (1999) Methods in Molecular Biology 106:247-283); RNAse protection assays (Hod (1992) Biotechniques 13:852-854); and reverse transcription polymerase chain reaction (RT-PCR) (Weis et al. (1992) Trends in Genetics 8:263-264). Alternatively, antibodies may be employed that can recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), gene expression analysis by massively parallel signature sequencing (MPSS) and/or next generation sequencing.

### RT-PCR

Reverse transcription polymerase chain reaction (RT-PCR) is a variant of polymerase chain reaction (PCR). According to this technique, a RNA strand is reverse transcribed into its DNA complement (i.e., complementary DNA, or cDNA) using the enzyme reverse transcriptase, and the resulting cDNA is amplified using PCR. Real-time polymerase chain reaction is another PCR variant, which is also referred to as quantitative PCR, Q-PCR, qRT-PCR, or sometimes as RT-PCR. Either the reverse transcription PCR method or the real-time PCR method can be used for molecular profiling according to the invention, and RT-PCR can refer to either unless otherwise specified or as understood by one of skill in the art.

RT-PCR can be used to determine RNA levels, e.g., mRNA or miRNA levels, of the biomarkers of the invention. RT-PCR can be used to compare such RNA levels of the biomarkers of the invention in different sample populations, in normal and tumor tissues, with or without drug treatment, to characterize patterns of gene expression, to discriminate between closely related RNAs, and to analyze RNA structure.

The first step is the isolation of RNA, e.g., mRNA, from a sample. The starting material can be total RNA isolated from human tumors or tumor cell lines, and corresponding normal tissues or cell lines, respectively. Thus RNA can be isolated from a sample, e.g., tumor cells or tumor cell lines, and compared with pooled DNA from healthy donors. If the source of mRNA is a primary tumor, mRNA can be extracted, for example, from frozen or archived paraffin-embedded and fixed (e.g. formalin-fixed) tissue samples.

General methods for mRNA extraction are well known in the art and are disclosed in standard textbooks of molecular biology, including Ausubel et al. (1997) Current Protocols of Molecular Biology, John Wiley and Sons. Methods for RNA extraction from paraffin embedded tissues are disclosed, for example, in Rupp & Locker (1987) Lab Invest. 56:A67, and De Andres et al., BioTechniques 18:42044 (1995). In particular, RNA isolation can be performed using purification kit, buffer set and protease from commercial manufacturers, such as Qiagen, according to the manufacturer's instructions (QIAGEN Inc., Valencia, CA). For example, total RNA from cells in culture can be isolated using Qiagen RNeasy mini-columns. Numerous RNA isolation kits are commercially available and can be used in the methods of the invention.

In the alternative, the first step is the isolation of miRNA from a target sample. The starting material is typically total RNA isolated from human tumors or tumor cell lines, and corresponding normal tissues or cell lines, respectively. Thus RNA can be isolated from a variety of primary tumors or tumor cell lines, with pooled DNA from healthy donors. If the source of miRNA is a primary tumor, miRNA can be extracted, for example, from frozen or archived paraffin-embedded and fixed (e.g. formalin-fixed) tissue samples.

General methods for miRNA extraction are well known in the art and are disclosed in standard textbooks of molecular biology, including Ausubel et al. (1997) Current Protocols of Molecular Biology, John Wiley and Sons. Methods for RNA extraction from paraffin embedded tissues are disclosed, for example, in Rupp & Locker (1987) Lab Invest. 56:A67, and De Andres et al., BioTechniques 18:42044 (1995). In particular, RNA isolation can be performed using purification kit, buffer set and protease from commercial manufacturers, such as Qiagen, according to the manufacturer's instructions. For example, total RNA from cells in culture can be isolated using Qiagen RNeasy mini-columns. Numerous miRNA isolation kits are commercially available and can be used in the methods of the invention.

Whether the RNA comprises mRNA, miRNA or other types of RNA, gene expression profiling by RT-PCR can include reverse transcription of the RNA template into cDNA, followed by amplification in a PCR reaction. Commonly used reverse transcriptases include, but are not limited to, avilo myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. TaqMan PCR typically uses the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TaqMan™ RT-PCR can be performed using commercially available equipment, such as, for example, ABI PRISM 7700™ Sequence Detection System™ (Perkin-Elmer-Applied Biosystems, Foster City, Calif., USA), or LightCycler (Roche Molecular Biochemicals, Mannheim, Germany). In one specific embodiment, the 5′ nuclease procedure is run on a real-time quantitative PCR device such as the ABI PRISM 7700 Sequence Detection System. The system consists of a thermocycler, laser, charge-coupled device (CCD), camera and computer. The system amplifies samples in a 96-well format on a thermocycler. During amplification, laser-induced fluorescent signal is collected in real-time through fiber optic cables for all 96 wells, and detected at the CCD. The system includes software for running the instrument and for analyzing the data.

TaqMan data are initially expressed as Ct, or the threshold cycle. As discussed above, fluorescence values are recorded during every cycle and represent the amount of product amplified to that point in the amplification reaction. The point when the fluorescent signal is first recorded as statistically significant is the threshold cycle (Ct).

To minimize errors and the effect of sample-to-sample variation, RT-PCR is usually performed using an internal standard. The ideal internal standard is expressed at a constant level among different tissues, and is unaffected by the experimental treatment. RNAs most frequently used to normalize patterns of gene expression are mRNAs for the housekeeping genes glyceraldehyde-3-phosphate-dehydrogenase (GAPDH) and β-actin.

Real time quantitative PCR (also quantitative real time polymerase chain reaction, QRT-PCR or Q-PCR) is a more recent variation of the RT-PCR technique. Q-PCR can measure PCR product accumulation through a dual-labeled fluorigenic probe (i.e., TaqMan probe). Real time PCR is compatible both with quantitative competitive PCR, where internal competitor for each target sequence is used for normalization, and with quantitative comparative PCR using a normalization gene contained within the sample, or a housekeeping gene for RT-PCR. See, e.g. Held et al. (1996) Genome Research 6:986-994.

Protein-based detection techniques are also useful for molecular profiling, especially when the nucleotide variant causes amino acid substitutions or deletions or insertions or frame shift that affect the protein primary, secondary or tertiary structure. To detect the amino acid variations, protein sequencing techniques may be used. For example, a protein or fragment thereof corresponding to a gene can be synthesized by recombinant expression using a DNA fragment isolated from an individual to be tested. Preferably, a cDNA fragment of no more than 100 to 150 base pairs encompassing the polymorphic locus to be determined is used. The amino acid sequence of the peptide can then be determined by conventional protein sequencing methods. Alternatively, the HPLC-microscopy tandem mass spectrometry technique can be used for determining the amino acid sequence variations. In this technique, proteolytic digestion is performed on a protein, and the resulting peptide mixture is separated by reversed-phase chromatographic separation. Tandem mass spectrometry is then performed and the data collected is analyzed. See Gatlin et al., Anal. Chem., 72:757-763 (2000).

### Microarray

The biomarkers of the invention can also be identified, confirmed, and/or measured using the microarray technique. Thus, the expression profile biomarkers can be measured in cancer samples using microarray technology. In this method, polynucleotide sequences of interest are plated, or arrayed, on a microchip substrate. The arrayed sequences are then hybridized with specific DNA probes from cells or tissues of interest. The source of mRNA can be total RNA isolated from a sample, e.g., human tumors or tumor cell lines and corresponding normal tissues or cell lines. Thus RNA can be isolated from a variety of primary tumors or tumor cell lines. If the source of mRNA is a primary tumor, mRNA can be extracted, for example, from frozen or archived paraffin-embedded and fixed (e.g. formalin-fixed) tissue samples, which are routinely prepared and preserved in everyday clinical practice.

The expression profile of biomarkers can be measured in either fresh or paraffin-embedded tumor tissue, or body fluids using microarray technology. In this method, polynucleotide sequences of interest are plated, or arrayed, on a microchip substrate. The arrayed sequences are then hybridized with specific DNA probes from cells or tissues of interest. As with the RT-PCR method, the source of miRNA typically is total RNA isolated from human tumors or tumor cell lines, including body fluids, such as serum, urine, tears, and exosomes and corresponding normal tissues or cell lines. Thus RNA can be isolated from a variety of sources. If the source of miRNA is a primary tumor, miRNA can be extracted, for example, from frozen tissue samples, which are routinely prepared and preserved in everyday clinical practice.

Also known as biochip, DNA chip, or gene array, cDNA microarray technology allows for identification of gene expression levels in a biologic sample. cDNAs or oligonucleotides, each representing a given gene, are immobilized on a substrate, e.g., a small chip, bead or nylon membrane, tagged, and serve as probes that will indicate whether they are expressed in biologic samples of interest. The simultaneous expression of thousands of genes can be monitored simultaneously.

In a specific embodiment of the microarray technique, PCR amplified inserts of cDNA clones are applied to a substrate in a dense array. In one aspect, at least 100, 200, 300, 400, 500, 600, 700, 800, 900, 1,000, 1,500, 2,000, 3000, 4000, 5000, 6000, 7000, 8000, 9000, 10,000, 15,000, 20,000, 25,000, 30,000, 35,000, 40,000, 45,000 or at least 50,000 nucleotide sequences are applied to the substrate. Each sequence can correspond to a different gene, or multiple sequences can be arrayed per gene. The microarrayed genes, immobilized on the microchip, are suitable for hybridization under stringent conditions. Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the chip is scanned by confocal laser microscopy or by another detection method, such as a CCD camera. Quantitation of hybridization of each arrayed element allows for assessment of corresponding mRNA abundance. With dual color fluorescence, separately labeled cDNA probes generated from two sources of RNA are hybridized pairwise to the array. The relative abundance of the transcripts from the two sources corresponding to each specified gene is thus determined simultaneously. The miniaturized scale of the hybridization affords a convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels (Schena et al. (1996) Proc. Natl. Acad. Sci. USA 93(2):106-149). Microarray analysis can be performed by commercially available equipment following manufacturer's protocols, including without limitation the Affymetrix GeneChip technology (Affymetrix, Santa Clara, CA), Agilent (Agilent Technologies, Inc., Santa Clara, CA), or Illumina (Illumina, Inc., San Diego, CA) microarray technology.

The development of microarray methods for large-scale analysis of gene expression makes it possible to search systematically for molecular markers of cancer classification and outcome prediction in a variety of tumor types.

In some embodiments, the Agilent Whole Human Genome Microarray Kit (Agilent Technologies, Inc., Santa Clara, CA). The system can analyze more than 41,000 unique human genes and transcripts represented, all with public domain annotations. The system is used according to the manufacturer's instructions.

In some embodiments, the Illumina Whole Genome DASL assay (Illumina Inc., San Diego, CA) is used. The system offers a method to simultaneously profile over 24,000 transcripts from minimal RNA input, from both fresh frozen (FF) and formalin-fixed paraffin embedded (FFPE) tissue sources, in a high throughput fashion.

Microarray expression analysis comprises identifying whether a gene or gene product is up-regulated or down-regulated relative to a reference. The identification can be performed using a statistical test to determine statistical significance of any differential expression observed. In some embodiments, statistical significance is determined using a parametric statistical test. The parametric statistical test can comprise, for example, a fractional factorial design, analysis of variance (ANOVA), a t-test, least squares, a Pearson correlation, simple linear regression, nonlinear regression, multiple linear regression, or multiple nonlinear regression. Alternatively, the parametric statistical test can comprise a one-way analysis of variance, two-way analysis of variance, or repeated measures analysis of variance. In other embodiments, statistical significance is determined using a nonparametric statistical test. Examples include, but are not limited to, a Wilcoxon signed-rank test, a Mann-Whitney test, a Kruskal-Wallis test, a Friedman test, a Spearman ranked order correlation coefficient, a Kendall Tau analysis, and a nonparametric regression test. In some embodiments, statistical significance is determined at a p-value of less than about 0.05, 0.01, 0.005, 0.001, 0.0005, or 0.0001. Although the microarray systems used in the methods of the invention may assay thousands of transcripts, data analysis need only be performed on the transcripts of interest, thereby reducing the problem of multiple comparisons inherent in performing multiple statistical tests. The p-values can also be corrected for multiple comparisons, e.g., using a Bonferroni correction, a modification thereof, or other technique known to those in the art, e.g., the Hochberg correction, Holm-Bonferroni correction, S̆idák correction, or Dunnett's correction. The degree of differential expression can also be taken into account. For example, a gene can be considered as differentially expressed when the fold-change in expression compared to control level is at least 1.2, 1.3, 1.4, 1.5, 1.6, 1.7, 1.8, 1.9, 2.0, 2.2, 2.5, 2.7, 3.0, 4, 5, 6, 7, 8, 9 or 10-fold different in the sample versus the control. The differential expression takes into account both overexpression and underexpression. A gene or gene product can be considered up or down-regulated if the differential expression meets a statistical threshold, a fold-change threshold, or both. For example, the criteria for identifying differential expression can comprise both a p-value of 0.001 and fold change of at least 1.5-fold (up or down). One of skill will understand that such statistical and threshold measures can be adapted to determine differential expression by any molecular profiling technique disclosed herein.

Various methods of the invention make use of many types of microarrays that detect the presence and potentially the amount of biological entities in a sample. Arrays typically contain addressable moieties that can detect the presence of the entity in the sample, e.g., via a binding event. Microarrays include without limitation DNA microarrays, such as cDNA microarrays, oligonucleotide microarrays and SNP microarrays, microRNA arrays, protein microarrays, antibody microarrays, tissue microarrays, cellular microarrays (also called transfection microarrays), chemical compound microarrays, and carbohydrate arrays (glycoarrays). DNA arrays typically comprise addressable nucleotide sequences that can bind to sequences present in a sample. MicroRNA arrays, e.g., the MMChips array from the University of Louisville or commercial systems from Agilent, can be used to detect microRNAs. Protein microarrays can be used to identify protein-protein interactions, including without limitation identifying substrates of protein kinases, transcription factor protein-activation, or to identify the targets of biologically active small molecules. Protein arrays may comprise an array of different protein molecules, commonly antibodies, or nucleotide sequences that bind to proteins of interest. Antibody microarrays comprise antibodies spotted onto the protein chip that are used as capture molecules to detect proteins or other biological materials from a sample, e.g., from cell or tissue lysate solutions. For example, antibody arrays can be used to detect biomarkers from bodily fluids, e.g., serum or urine, for diagnostic applications. Tissue microarrays comprise separate tissue cores assembled in array fashion to allow multiplex histological analysis. Cellular microarrays, also called transfection microarrays, comprise various capture agents, such as antibodies, proteins, or lipids, which can interact with cells to facilitate their capture on addressable locations. Chemical compound microarrays comprise arrays of chemical compounds and can be used to detect protein or other biological materials that bind the compounds. Carbohydrate arrays (glycoarrays) comprise arrays of carbohydrates and can detect, e.g., protein that bind sugar moieties. One of skill will appreciate that similar technologies or improvements can be used according to the methods of the invention.

Certain embodiments of the current methods comprise a multi-well reaction vessel, including without limitation, a multi-well plate or a multi-chambered microfluidic device, in which a multiplicity of amplification reactions and, in some embodiments, detection are performed, typically in parallel. In certain embodiments, one or more multiplex reactions for generating amplicons are performed in the same reaction vessel, including without limitation, a multi-well plate, such as a 96-well, a 384-well, a 1536-well plate, and so forth; or a microfluidic device, for example but not limited to, a TaqMan™ Low Density Array (Applied Biosystems, Foster City, CA). In some embodiments, a massively parallel amplifying step comprises a multi-well reaction vessel, including a plate comprising multiple reaction wells, for example but not limited to, a 24-well plate, a 96-well plate, a 384-well plate, or a 1536-well plate; or a multi-chamber microfluidics device, for example but not limited to a low density array wherein each chamber or well comprises an appropriate primer(s), primer set(s), and/or reporter probe(s), as appropriate. Typically such amplification steps occur in a series of parallel single-plex, two-plex, three-plex, four-plex, five-plex, or six-plex reactions, although higher levels of parallel multiplexing are also within the intended scope of the current teachings. These methods can comprise PCR methodology, such as RT-PCR, in each of the wells or chambers to amplify and/or detect nucleic acid molecules of interest.

Low density arrays can include arrays that detect 10s or 100s of molecules as opposed to 1000s of molecules. These arrays can be more sensitive than high density arrays. In embodiments, a low density array such as a TaqMan™ Low Density Array is used to detect one or more gene or gene product in any of Tables 5-12. For example, the low density array can be used to detect at least 1, 2, 3, 4, 5, 6, 7, 8, 9, 10, 15, 20, 25, 30, 40, 50, 60, 70, 80, 90 or 100 genes or gene products selected from any of Tables 5-12.

In some embodiments, the disclosed methods comprise a microfluidics device, “lab on a chip,” or micrototal analytical system (pTAS). In some embodiments, sample preparation is performed using a microfluidics device. In some embodiments, an amplification reaction is performed using a microfluidics device. In some embodiments, a sequencing or PCR reaction is performed using a microfluidic device. In some embodiments, the nucleotide sequence of at least a part of an amplified product is obtained using a microfluidics device. In some embodiments, detecting comprises a microfluidic device, including without limitation, a low density array, such as a TaqMan™ Low Density Array. Descriptions of exemplary microfluidic devices can be found in, among other places, Published PCT Application Nos. WO/0185341 and WO 04/011666; Kartalov and Quake, Nucl. Acids Res. 32:2873-79, 2004; and Fiorini and Chiu, Bio Techniques 38:429-46, 2005.

Any appropriate microfluidic device can be used in the methods of the invention. Examples of microfluidic devices that may be used, or adapted for use with molecular profiling, include but are not limited to those described in U.S. Pat. Nos. 7,591,936, 7,581,429, 7,579,136, 7,575,722, 7,568,399, 7,552,741, 7,544,506, 7,541,578, 7,518,726, 7,488,596, 7,485,214, 7,467,928, 7,452,713, 7,452,509, 7,449,096, 7,431,887, 7,422,725, 7,422,669, 7,419,822, 7,419,639, 7,413,709, 7,411,184, 7,402,229, 7,390,463, 7,381,471, 7,357,864, 7,351,592, 7,351,380, 7,338,637, 7,329,391, 7,323,140, 7,261,824, 7,258,837, 7,253,003, 7,238,324, 7,238,255, 7,233,865, 7,229,538, 7,201,881, 7,195,986, 7,189,581, 7,189,580, 7,189,368, 7,141,978, 7,138,062, 7,135,147, 7,125,711, 7,118,910, 7,118,661, 7,640,947, 7,666,361, 7,704,735; U.S. Patent Application Publication 20060035243; and International Patent Publication WO 2010/072410; each of which patents or applications are incorporated herein by reference in their entirety. Another example for use with methods disclosed herein is described in Chen et al., “Microfluidic isolation and transcriptome analysis of serum vesicles,” Lab on a Chip, Dec. 8, 2009 DOI: 10.1039/b916199f.

### Gene Expression Analysis by Massively Parallel Signature Sequencing (MPSS)

This method, described by Brenner et al. (2000) Nature Biotechnology 18:630-634, is a sequencing approach that combines non-gel-based signature sequencing with in vitro cloning of millions of templates on separate microbeads. First, a microbead library of DNA templates is constructed by in vitro cloning. This is followed by the assembly of a planar array of the template-containing microbeads in a flow cell at a high density. The free ends of the cloned templates on each microbead are analyzed simultaneously, using a fluorescence-based signature sequencing method that does not require DNA fragment separation. This method has been shown to simultaneously and accurately provide, in a single operation, hundreds of thousands of gene signature sequences from a cDNA library.

MPSS data has many uses. The expression levels of nearly all transcripts can be quantitatively determined; the abundance of signatures is representative of the expression level of the gene in the analyzed tissue. Quantitative methods for the analysis of tag frequencies and detection of differences among libraries have been published and incorporated into public databases for SAGE™ data and are applicable to MPSS data. The availability of complete genome sequences permits the direct comparison of signatures to genomic sequences and further extends the utility of MPSS data. Because the targets for MPSS analysis are not pre-selected (like on a microarray), MPSS data can characterize the full complexity of transcriptomes. This is analogous to sequencing millions of ESTs at once, and genomic sequence data can be used so that the source of the MPSS signature can be readily identified by computational means.

### Serial Analysis of Gene Expression (SAGE)

Serial analysis of gene expression (SAGE) is a method that allows the simultaneous and quantitative analysis of a large number of gene transcripts, without the need of providing an individual hybridization probe for each transcript. First, a short sequence tag (e.g., about 10-14 bp) is generated that contains sufficient information to uniquely identify a transcript, provided that the tag is obtained from a unique position within each transcript. Then, many transcripts are linked together to form long serial molecules, that can be sequenced, revealing the identity of the multiple tags simultaneously. The expression pattern of any population of transcripts can be quantitatively evaluated by determining the abundance of individual tags, and identifying the gene corresponding to each tag. See, e.g. Velculescu et al. (1995) Science 270:484-487; and Velculescu et al. (1997) Cell 88:243-51.

### DNA Copy Number Profiling

Any method capable of determining a DNA copy number profile of a particular sample can be used for molecular profiling according to the invention as long as the resolution is sufficient to identify the biomarkers of the invention. The skilled artisan is aware of and capable of using a number of different platforms for assessing whole genome copy number changes at a resolution sufficient to identify the copy number of the one or more biomarkers of the invention. Some of the platforms and techniques are described in the embodiments below. In some embodiments of the invention, ISH techniques as described herein are also used for determining copy number/gene amplification.

In some embodiments, the copy number profile analysis involves amplification of whole genome DNA by a whole genome amplification method. The whole genome amplification method can use a strand displacing polymerase and random primers.

In some aspects of these embodiments, the copy number profile analysis involves hybridization of whole genome amplified DNA with a high density array. In a more specific aspect, the high density array has 5,000 or more different probes. In another specific aspect, the high density array has 5,000, 10,000, 20,000, 50,000, 100,000, 200,000, 300,000, 400,000, 500,000, 600,000, 700,000, 800,000, 900,000, or 1,000,000 or more different probes. In another specific aspect, each of the different probes on the array is an oligonucleotide having from about 15 to 200 bases in length. In another specific aspect, each of the different probes on the array is an oligonucleotide having from about 15 to 200, 15 to 150, 15 to 100, 15 to 75, 15 to 60, or 20 to 55 bases in length.

In some embodiments, a microarray is employed to aid in determining the copy number profile for a sample, e.g., cells from a tumor. Microarrays typically comprise a plurality of oligomers (e.g., DNA or RNA polynucleotides or oligonucleotides, or other polymers), synthesized or deposited on a substrate (e.g., glass support) in an array pattern. The support-bound oligomers are “probes”, which function to hybridize or bind with a sample material (e.g., nucleic acids prepared or obtained from the tumor samples), in hybridization experiments. The reverse situation can also be applied: the sample can be bound to the microarray substrate and the oligomer probes are in solution for the hybridization. In use, the array surface is contacted with one or more targets under conditions that promote specific, high-affinity binding of the target to one or more of the probes. In some configurations, the sample nucleic acid is labeled with a detectable label, such as a fluorescent tag, so that the hybridized sample and probes are detectable with scanning equipment. DNA array technology offers the potential of using a multitude (e.g., hundreds of thousands) of different oligonucleotides to analyze DNA copy number profiles. In some embodiments, the substrates used for arrays are surface-derivatized glass or silica, or polymer membrane surfaces (see e.g., in Z. Guo, et al., Nucleic Acids Res, 22, 5456-65 (1994); U. Maskos, E. M. Southern, Nucleic Acids Res, 20, 1679-84 (1992), and E. M. Southern, et al., Nucleic Acids Res, 22, 1368-73 (1994), each incorporated by reference herein). Modification of surfaces of array substrates can be accomplished by many techniques. For example, siliceous or metal oxide surfaces can be derivatized with bifunctional silanes, i.e., silanes having a first functional group enabling covalent binding to the surface (e.g., Si-halogen or Si-alkoxy group, as in—SiCl₃ or —Si(OCH₃)₃, respectively) and a second functional group that can impart the desired chemical and/or physical modifications to the surface to covalently or non-covalently attach ligands and/or the polymers or monomers for the biological probe array. Silylated derivatizations and other surface derivatizations that are known in the art (see for example U.S. Pat. No. 5,624,711 to Sundberg, U.S. Pat. No. 5,266,222 to Willis, and U.S. Pat. No. 5,137,765 to Farnsworth, each incorporated by reference herein). Other processes for preparing arrays are described in U.S. Pat. No. 6,649,348, to Bass et. al., assigned to Agilent Corp., which disclose DNA arrays created by in situ synthesis methods.

Polymer array synthesis is also described extensively in the literature including in the following: WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, 5,412,087, 6,147,205, 6,262,216, 6,310,189, 5,889,165, and 5,959,098 in PCT Applications Nos. PCT/US99/00730 (International Publication No. WO 99/36760) and PCT/US01/04285 (International Publication No. WO 01/58593), which are all incorporated herein by reference in their entirety for all purposes.

Nucleic acid arrays that are useful in the present invention include, but are not limited to, those that are commercially available from Affymetrix (Santa Clara, Calif) under the brand name GeneChip™. Example arrays are shown on the website at affymetrix.com. Another microarray supplier is Illumina, Inc., of San Diego, Calif. with example arrays shown on their website at illumina.com.

In some embodiments, the inventive methods provide for sample preparation. Depending on the microarray and experiment to be performed, sample nucleic acid can be prepared in a number of ways by methods known to the skilled artisan. In some aspects of the invention, prior to or concurrent with genotyping (analysis of copy number profiles), the sample may be amplified any number of mechanisms. The most common amplification procedure used involves PCR. See, for example, PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188, and 5,333,675, and each of which is incorporated herein by reference in their entireties for all purposes. In some embodiments, the sample may be amplified on the array (e.g., U.S. Pat. No. 6,300,070 which is incorporated herein by reference)

Other suitable amplification methods include the ligase chain reaction (LCR) (for example, Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. Nos. 6,361,947, 6,391,592 and U.S. Ser. Nos. 09/916,135, 09/920,491 (U.S. Patent Application Publication 20030096235), U.S. Ser. No. 09/910,292 (U.S. Patent Application Publication 20030082543), and U.S. Ser. No. 10/013,598.

Methods for conducting polynucleotide hybridization assays are well developed in the art. Hybridization assay procedures and conditions used in the methods of the invention will vary depending on the application and are selected in accordance with the general binding methods known including those referred to in: Maniatis et al. Molecular Cloning: A Laboratory Manual (2.sup.nd Ed. Cold Spring Harbor, N.Y., 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference.

The methods of the invention may also involve signal detection of hybridization between ligands in after (and/or during) hybridization. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. Ser. No. 10/389,194 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Ser. Nos. 10/389,194, 60/493,495 and in PCT Application PCT/US99/06097 (published as WO99/47964), each of which also is hereby incorporated by reference in its entirety for all purposes.

### Immuno-based Assays

Protein-based detection molecular profiling techniques include immunoaffinity assays based on antibodies selectively immunoreactive with mutant gene encoded protein according to the present invention. These techniques include without limitation immunoprecipitation, Western blot analysis, molecular binding assays, enzyme-linked immunosorbent assay (ELISA), enzyme-linked immunofiltration assay (ELIFA), fluorescence activated cell sorting (FACS) and the like. For example, an optional method of detecting the expression of a biomarker in a sample comprises contacting the sample with an antibody against the biomarker, or an immunoreactive fragment of the antibody thereof, or a recombinant protein containing an antigen binding region of an antibody against the biomarker; and then detecting the binding of the biomarker in the sample. Methods for producing such antibodies are known in the art. Antibodies can be used to immunoprecipitate specific proteins from solution samples or to immunoblot proteins separated by, e.g., polyacrylamide gels. Immunocytochemical methods can also be used in detecting specific protein polymorphisms in tissues or cells. Other well-known antibody-based techniques can also be used including, e.g., ELISA, radioimmunoassay (RIA), immunoradiometric assays (IRMA) and immunoenzymatic assays (IEMA), including sandwich assays using monoclonal or polyclonal antibodies. See, e.g., U.S. Pat. Nos. 4,376,110 and 4,486,530, both of which are incorporated herein by reference.

In alternative methods, the sample may be contacted with an antibody specific for a biomarker under conditions sufficient for an antibody-biomarker complex to form, and then detecting said complex. The presence of the biomarker may be detected in a number of ways, such as by Western blotting and ELISA procedures for assaying a wide variety of tissues and samples, including plasma or serum. A wide range of immunoassay techniques using such an assay format are available, see, e.g., U.S. Pat. Nos. 4,016,043, 4,424,279 and 4,018,653. These include both single-site and two-site or “sandwich” assays of the non-competitive types, as well as in the traditional competitive binding assays. These assays also include direct binding of a labelled antibody to a target biomarker.

A number of variations of the sandwich assay technique exist, and all are intended to be encompassed by the present invention. Briefly, in a typical forward assay, an unlabelled antibody is immobilized on a solid substrate, and the sample to be tested brought into contact with the bound molecule. After a suitable period of incubation, for a period of time sufficient to allow formation of an antibody-antigen complex, a second antibody specific to the antigen, labelled with a reporter molecule capable of producing a detectable signal is then added and incubated, allowing time sufficient for the formation of another complex of antibody-antigen-labelled antibody. Any unreacted material is washed away, and the presence of the antigen is determined by observation of a signal produced by the reporter molecule. The results may either be qualitative, by simple observation of the visible signal, or may be quantitated by comparing with a control sample containing known amounts of biomarker.

Variations on the forward assay include a simultaneous assay, in which both sample and labelled antibody are added simultaneously to the bound antibody. These techniques are well known to those skilled in the art, including any minor variations as will be readily apparent. In a typical forward sandwich assay, a first antibody having specificity for the biomarker is either covalently or passively bound to a solid surface. The solid surface is typically glass or a polymer, the most commonly used polymers being cellulose, polyacrylamide, nylon, polystyrene, polyvinyl chloride or polypropylene. The solid supports may be in the form of tubes, beads, discs of microplates, or any other surface suitable for conducting an immunoassay. The binding processes are well-known in the art and generally consist of cross-linking covalently binding or physically adsorbing, the polymer-antibody complex is washed in preparation for the test sample. An aliquot of the sample to be tested is then added to the solid phase complex and incubated for a period of time sufficient (e.g. 2-40 minutes or overnight if more convenient) and under suitable conditions (e.g. from room temperature to 40° C. such as between 25° C. and 32° C. inclusive) to allow binding of any subunit present in the antibody. Following the incubation period, the antibody subunit solid phase is washed and dried and incubated with a second antibody specific for a portion of the biomarker. The second antibody is linked to a reporter molecule which is used to indicate the binding of the second antibody to the molecular marker.

An alternative method involves immobilizing the target biomarkers in the sample and then exposing the immobilized target to specific antibody which may or may not be labelled with a reporter molecule. Depending on the amount of target and the strength of the reporter molecule signal, a bound target may be detectable by direct labelling with the antibody. Alternatively, a second labelled antibody, specific to the first antibody is exposed to the target-first antibody complex to form a target-first antibody-second antibody tertiary complex. The complex is detected by the signal emitted by the reporter molecule. By “reporter molecule”, as used in the present specification, is meant a molecule which, by its chemical nature, provides an analytically identifiable signal which allows the detection of antigen-bound antibody. The most commonly used reporter molecules in this type of assay are either enzymes, fluorophores or radionuclide containing molecules (i.e. radioisotopes) and chemiluminescent molecules.

In the case of an enzyme immunoassay, an enzyme is conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, $3-galactosidase and alkaline phosphatase, amongst others. The substrates to be used with the specific enzymes are generally chosen for the production, upon hydrolysis by the corresponding enzyme, of a detectable color change. Examples of suitable enzymes include alkaline phosphatase and peroxidase. It is also possible to employ fluorogenic substrates, which yield a fluorescent product rather than the chromogenic substrates noted above. In all cases, the enzyme-labelled antibody is added to the first antibody-molecular marker complex, allowed to bind, and then the excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of biomarker which was present in the sample. Alternately, fluorescent compounds, such as fluorescein and rhodamine, may be chemically coupled to antibodies without altering their binding capacity. When activated by illumination with light of a particular wavelength, the fluorochrome-labelled antibody adsorbs the light energy, inducing a state to excitability in the molecule, followed by emission of the light at a characteristic color visually detectable with a light microscope. As in the EIA, the fluorescent labelled antibody is allowed to bind to the first antibody-molecular marker complex. After washing off the unbound reagent, the remaining tertiary complex is then exposed to the light of the appropriate wavelength, the fluorescence observed indicates the presence of the molecular marker of interest. Immunofluorescence and EIA techniques are both very well established in the art. However, other reporter molecules, such as radioisotope, chemiluminescent or bioluminescent molecules, may also be employed.

### Immunohistochemistry (IHC)

IHC is a process of localizing antigens (e.g., proteins) in cells of a tissue binding antibodies specifically to antigens in the tissues. The antigen-binding antibody can be conjugated or fused to a tag that allows its detection, e.g., via visualization. In some embodiments, the tag is an enzyme that can catalyze a color-producing reaction, such as alkaline phosphatase or horseradish peroxidase. The enzyme can be fused to the antibody or non-covalently bound, e.g., using a biotin-avadin system. Alternatively, the antibody can be tagged with a fluorophore, such as fluorescein, rhodamine, DyLight Fluor or Alexa Fluor. The antigen-binding antibody can be directly tagged or it can itself be recognized by a detection antibody that carries the tag. Using IHC, one or more proteins may be detected. The expression of a gene product can be related to its staining intensity compared to control levels. In some embodiments, the gene product is considered differentially expressed if its staining varies at least 1.2, 1.3, 1.4, 1.5, 1.6, 1.7, 1.8, 1.9, 2.0, 2.2, 2.5, 2.7, 3.0, 4, 5, 6, 7, 8, 9 or 10-fold in the sample versus the control.

IHC comprises the application of antigen-antibody interactions to histochemical techniques. In an illustrative example, a tissue section is mounted on a slide and is incubated with antibodies (polyclonal or monoclonal) specific to the antigen (primary reaction). The antigen-antibody signal is then amplified using a second antibody conjugated to a complex of peroxidase antiperoxidase (PAP), avidin-biotin-peroxidase (ABC) or avidin-biotin alkaline phosphatase. In the presence of substrate and chromogen, the enzyme forms a colored deposit at the sites of antibody-antigen binding. Immunofluorescence is an alternate approach to visualize antigens. In this technique, the primary antigen-antibody signal is amplified using a second antibody conjugated to a fluorochrome. On UV light absorption, the fluorochrome emits its own light at a longer wavelength (fluorescence), thus allowing localization of antibody-antigen complexes.

### Epigenetic Status

Molecular profiling methods according to the invention also comprise measuring epigenetic change, i.e., modification in a gene caused by an epigenetic mechanism, such as a change in methylation status or histone acetylation. Frequently, the epigenetic change will result in an alteration in the levels of expression of the gene which may be detected (at the RNA or protein level as appropriate) as an indication of the epigenetic change. Often the epigenetic change results in silencing or down regulation of the gene, referred to as “epigenetic silencing.” The most frequently investigated epigenetic change in the methods of the invention involves determining the DNA methylation status of a gene, where an increased level of methylation is typically associated with the relevant cancer (since it may cause down regulation of gene expression). Aberrant methylation, which may be referred to as hypermethylation, of the gene or genes can be detected. Typically, the methylation status is determined in suitable CpG islands which are often found in the promoter region of the gene(s). The term “methylation,” “methylation state” or “methylation status” may refers to the presence or absence of 5-methylcytosine at one or a plurality of CpG dinucleotides within a DNA sequence. CpG dinucleotides are typically concentrated in the promoter regions and exons of human genes.

Diminished gene expression can be assessed in terms of DNA methylation status or in terms of expression levels as determined by the methylation status of the gene. One method to detect epigenetic silencing is to determine that a gene which is expressed in normal cells is less expressed or not expressed in tumor cells. Accordingly, the invention provides for a method of molecular profiling comprising detecting epigenetic silencing.

Various assay procedures to directly detect methylation are known in the art, and can be used in conjunction with the present invention. These assays rely onto two distinct approaches: bisulphite conversion based approaches and non-bisulphite based approaches. Non-bisulphite based methods for analysis of DNA methylation rely on the inability of methylation-sensitive enzymes to cleave methylation cytosines in their restriction. The bisulphite conversion relies on treatment of DNA samples with sodium bisulphite which converts unmethylated cytosine to uracil, while methylated cytosines are maintained (Furuichi Y, Wataya Y, Hayatsu H, Ukita T. Biochem Biophys Res Commun. 1970 Dec. 9; 41(5):1185-91). This conversion results in a change in the sequence of the original DNA. Methods to detect such changes include MS AP-PCR (Methylation-Sensitive Arbitrarily-Primed Polymerase Chain Reaction), a technology that allows for a global scan of the genome using CG-rich primers to focus on the regions most likely to contain CpG dinucleotides, and described by Gonzalgo et al., Cancer Research 57:594-599, 1997; MethyLight™, which refers to the art-recognized fluorescence-based real-time PCR technique described by Eads et al., Cancer Res. 59:2302-2306, 1999; the HeavyMethyl™assay, in the embodiment thereof implemented herein, is an assay, wherein methylation specific blocking probes (also referred to herein as blockers) covering CpG positions between, or covered by the amplification primers enable methylation-specific selective amplification of a nucleic acid sample; HeavyMethyl™MethyLight™ is a variation of the MethyLight™ assay wherein the MethyLight™ assay is combined with methylation specific blocking probes covering CpG positions between the amplification primers; Ms-SNuPE (Methylation-sensitive Single Nucleotide Primer Extension) is an assay described by Gonzalgo & Jones, Nucleic Acids Res. 25:2529-2531, 1997; MSP (Methylation-specific PCR) is a methylation assay described by Herman et al. Proc. Natl. Acad. Sci. USA 93:9821-9826, 1996, and by U.S. Pat. No. 5,786,146; COBRA (Combined Bisulfite Restriction Analysis) is a methylation assay described by Xiong & Laird, Nucleic Acids Res. 25:2532-2534, 1997; MCA (Methylated CpG Island Amplification) is a methylation assay described by Toyota et al., Cancer Res. 59:2307-12, 1999, and in WO 00/26401A1.

Other techniques for DNA methylation analysis include sequencing, methylation-specific PCR (MS-PCR), melting curve methylation-specific PCR (McMS-PCR), MLPA with or without bisulfite treatment, QAMA, MSRE-PCR, MethyLight, ConLight-MSP, bisulfite conversion-specific methylation-specific PCR (BS-MSP), COBRA (which relies upon use of restriction enzymes to reveal methylation dependent sequence differences in PCR products of sodium bisulfite-treated DNA), methylation-sensitive single-nucleotide primer extension conformation (MS-SNuPE), methylation-sensitive single-strand conformation analysis (MS-SSCA), Melting curve combined bisulfite restriction analysis (McCOBRA), PyroMethA, HeavyMethyl, MALDI-TOF, MassARRAY, Quantitative analysis of methylated alleles (QAMA), enzymatic regional methylation assay (ERMA), QBSUPT, MethylQuant, Quantitative PCR sequencing and oligonucleotide-based microarray systems, Pyrosequencing, Meth-DOP-PCR. A review of some useful techniques is provided in Nucleic acids research, 1998, Vol. 26, No. 10, 2255-2264; Nature Reviews, 2003, Vol. 3, 253-266; Oral Oncology, 2006, Vol. 42, 5-13, which references are incorporated herein in their entirety. Any of these techniques may be used in accordance with the present invention, as appropriate. Other techniques are described in U.S. Patent Publications 20100144836; and 20100184027, which applications are incorporated herein by reference in their entirety.

Through the activity of various acetylases and deacetylylases the DNA binding function of histone proteins is tightly regulated. Furthermore, histone acetylation and histone deactelyation have been linked with malignant progression. See Nature, 429: 457-63, 2004. Methods to analyze histone acetylation are described in U.S. Patent Publications 20100144543 and 20100151468, which applications are incorporated herein by reference in their entirety.

### Sequence Analysis

Molecular profiling according to the present invention comprises methods for genotyping one or more biomarkers by determining whether an individual has one or more nucleotide variants (or amino acid variants) in one or more of the genes or gene products. Genotyping one or more genes according to the methods of the invention in some embodiments, can provide more evidence for selecting a treatment.

The biomarkers of the invention can be analyzed by any method useful for determining alterations in nucleic acids or the proteins they encode. According to one embodiment, the ordinary skilled artisan can analyze the one or more genes for mutations including deletion mutants, insertion mutants, frame shift mutants, nonsense mutants, missense mutant, and splice mutants.

Nucleic acid used for analysis of the one or more genes can be isolated from cells in the sample according to standard methodologies (Sambrook et al., 1989). The nucleic acid, for example, may be genomic DNA or fractionated or whole cell RNA, or miRNA acquired from exosomes or cell surfaces. Where RNA is used, it may be desired to convert the RNA to a complementary DNA. In one embodiment, the RNA is whole cell RNA; in another, it is poly-A RNA; in another, it is exosomal RNA. Normally, the nucleic acid is amplified. Depending on the format of the assay for analyzing the one or more genes, the specific nucleic acid of interest is identified in the sample directly using amplification or with a second, known nucleic acid following amplification. Next, the identified product is detected. In certain applications, the detection may be performed by visual means (e.g., ethidium bromide staining of a gel). Alternatively, the detection may involve indirect identification of the product via chemiluminescence, radioactive scintigraphy of radiolabel or fluorescent label or even via a system using electrical or thermal impulse signals (Affymax Technology; Bellus, 1994).

Various types of defects are known to occur in the biomarkers of the invention. Alterations include without limitation deletions, insertions, point mutations, and duplications. Point mutations can be silent or can result in stop codons, frame shift mutations or amino acid substitutions. Mutations in and outside the coding region of the one or more genes may occur and can be analyzed according to the methods of the invention. The target site of a nucleic acid of interest can include the region wherein the sequence varies. Examples include, but are not limited to, polymorphisms which exist in different forms such as single nucleotide variations, nucleotide repeats, multibase deletion (more than one nucleotide deleted from the consensus sequence), multibase insertion (more than one nucleotide inserted from the consensus sequence), microsatellite repeats (small numbers of nucleotide repeats with a typical 5-1000 repeat units), di-nucleotide repeats, tri-nucleotide repeats, sequence rearrangements (including translocation and duplication), chimeric sequence (two sequences from different gene origins are fused together), and the like. Among sequence polymorphisms, the most frequent polymorphisms in the human genome are single-base variations, also called single-nucleotide polymorphisms (SNPs). SNPs are abundant, stable and widely distributed across the genome.

Molecular profiling includes methods for haplotyping one or more genes. The haplotype is a set of genetic determinants located on a single chromosome and it typically contains a particular combination of alleles (all the alternative sequences of a gene) in a region of a chromosome. In other words, the haplotype is phased sequence information on individual chromosomes. Very often, phased SNPs on a chromosome define a haplotype. A combination of haplotypes on chromosomes can determine a genetic profile of a cell. It is the haplotype that determines a linkage between a specific genetic marker and a disease mutation. Haplotyping can be done by any methods known in the art. Common methods of scoring SNPs include hybridization microarray or direct gel sequencing, reviewed in Landgren et al., Genome Research, 8:769-776, 1998. For example, only one copy of one or more genes can be isolated from an individual and the nucleotide at each of the variant positions is determined. Alternatively, an allele specific PCR or a similar method can be used to amplify only one copy of the one or more genes in an individual, and the SNPs at the variant positions of the present invention are determined. The Clark method known in the art can also be employed for haplotyping. A high throughput molecular haplotyping method is also disclosed in Tost et al., Nucleic Acids Res., 30(19):e96 (2002), which is incorporated herein by reference.

Thus, additional variant(s) that are in linkage disequilibrium with the variants and/or haplotypes of the present invention can be identified by a haplotyping method known in the art, as will be apparent to a skilled artisan in the field of genetics and haplotyping. The additional variants that are in linkage disequilibrium with a variant or haplotype of the present invention can also be useful in the various applications as described below.

For purposes of genotyping and haplotyping, both genomic DNA and mRNA/cDNA can be used, and both are herein referred to generically as “gene.”

Numerous techniques for detecting nucleotide variants are known in the art and can all be used for the method of this invention. The techniques can be protein-based or nucleic acid-based. In either case, the techniques used must be sufficiently sensitive so as to accurately detect the small nucleotide or amino acid variations. Very often, a probe is used which is labeled with a detectable marker. Unless otherwise specified in a particular technique described below, any suitable marker known in the art can be used, including but not limited to, radioactive isotopes, fluorescent compounds, biotin which is detectable using streptavidin, enzymes (e.g., alkaline phosphatase), substrates of an enzyme, ligands and antibodies, etc. See Jablonski et al., Nucleic Acids Res., 14:6115-6128 (1986); Nguyen et al., Biotechniques, 13:116-123 (1992); Rigby et al., J. Mol. Biol., 113:237-251 (1977).

In a nucleic acid-based detection method, target DNA sample, i.e., a sample containing genomic DNA, cDNA, mRNA and/or miRNA, corresponding to the one or more genes must be obtained from the individual to be tested. Any tissue or cell sample containing the genomic DNA, miRNA, mRNA, and/or cDNA (or a portion thereof) corresponding to the one or more genes can be used. For this purpose, a tissue sample containing cell nucleus and thus genomic DNA can be obtained from the individual. Blood samples can also be useful except that only white blood cells and other lymphocytes have cell nucleus, while red blood cells are without a nucleus and contain only mRNA or miRNA. Nevertheless, miRNA and mRNA are also useful as either can be analyzed for the presence of nucleotide variants in its sequence or serve as template for cDNA synthesis. The tissue or cell samples can be analyzed directly without much processing. Alternatively, nucleic acids including the target sequence can be extracted, purified, and/or amplified before they are subject to the various detecting procedures discussed below. Other than tissue or cell samples, cDNAs or genomic DNAs from a cDNA or genomic DNA library constructed using a tissue or cell sample obtained from the individual to be tested are also useful.

To determine the presence or absence of a particular nucleotide variant, sequencing of the target genomic DNA or cDNA, particularly the region encompassing the nucleotide variant locus to be detected. Various sequencing techniques are generally known and widely used in the art including the Sanger method and Gilbert chemical method. The pyrosequencing method monitors DNA synthesis in real time using a luminometric detection system. Pyrosequencing has been shown to be effective in analyzing genetic polymorphisms such as single-nucleotide polymorphisms and can also be used in the present invention. See Nordstrom et al., Biotechnol. Appl. Biochem., 31(2):107-112 (2000); Ahmadian et al., Anal. Biochem., 280:103-110 (2000).

Nucleic acid variants can be detected by a suitable detection process. Non limiting examples of methods of detection, quantification, sequencing and the like are; mass detection of mass modified amplicons (e.g., matrix-assisted laser desorption ionization (MALDI) mass spectrometry and electrospray (ES) mass spectrometry), a primer extension method (e.g., iPLEX™; Sequenom, Inc.), microsequencing methods (e.g., a modification of primer extension methodology), ligase sequence determination methods (e.g., U.S. Pat. Nos. 5,679,524 and 5,952,174, and WO 01/27326), mismatch sequence determination methods (e.g., U.S. Pat. Nos. 5,851,770; 5,958,692; 6,110,684; and 6,183,958), direct DNA sequencing, fragment analysis (FA), restriction fragment length polymorphism (RFLP analysis), allele specific oligonucleotide (ASO) analysis, methylation-specific PCR (MSPCR), pyrosequencing analysis, acycloprime analysis, Reverse dot blot, GeneChip microarrays, Dynamic allele-specific hybridization (DASH), Peptide nucleic acid (PNA) and locked nucleic acids (LNA) probes, TaqMan, Molecular Beacons, Intercalating dye, FRET primers, AlphaScreen, SNPstream, genetic bit analysis (GBA), Multiplex minisequencing, SNaPshot, GOOD assay, Microarray miniseq, arrayed primer extension (APEX), Microarray primer extension (e.g., microarray sequence determination methods), Tag arrays, Coded microspheres, Template-directed incorporation (TDI), fluorescence polarization, Colorimetric oligonucleotide ligation assay (OLA), Sequence-coded OLA, Microarray ligation, Ligase chain reaction, Padlock probes, Invader assay, hybridization methods (e.g., hybridization using at least one probe, hybridization using at least one fluorescently labeled probe, and the like), conventional dot blot analyses, single strand conformational polymorphism analysis (SSCP, e.g., U.S. Pat. Nos. 5,891,625 and 6,013,499; Orita et al., Proc. Natl. Acad. Sci. U.S.A. 86: 27776-2770 (1989)), denaturing gradient gel electrophoresis (DGGE), heteroduplex analysis, mismatch cleavage detection, and techniques described in Sheffield et al., Proc. Natl. Acad. Sci. USA 49: 699-706 (1991), White et al., Genomics 12: 301-306 (1992), Grompe et al., Proc. Natl. Acad. Sci. USA 86: 5855-5892 (1989), and Grompe, Nature Genetics 5: 111-117 (1993), cloning and sequencing, electrophoresis, the use of hybridization probes and quantitative real time polymerase chain reaction (QRT-PCR), digital PCR, nanopore sequencing, chips and combinations thereof. The detection and quantification of alleles or paralogs can be carried out using the “closed-tube” methods described in U.S. patent application Ser. No. 11/950,395, filed on Dec. 4, 2007. In some embodiments the amount of a nucleic acid species is determined by mass spectrometry, primer extension, sequencing (e.g., any suitable method, for example nanopore or pyrosequencing), Quantitative PCR (Q-PCR or QRT-PCR), digital PCR, combinations thereof, and the like.

The term “sequence analysis” as used herein refers to determining a nucleotide sequence, e.g., that of an amplification product. The entire sequence or a partial sequence of a polynucleotide, e.g., DNA or mRNA, can be determined, and the determined nucleotide sequence can be referred to as a “read” or “sequence read.” For example, linear amplification products may be analyzed directly without further amplification in some embodiments (e.g., by using single-molecule sequencing methodology). In certain embodiments, linear amplification products may be subject to further amplification and then analyzed (e.g., using sequencing by ligation or pyrosequencing methodology). Reads may be subject to different types of sequence analysis. Any suitable sequencing method can be used to detect, and determine the amount of, nucleotide sequence species, amplified nucleic acid species, or detectable products generated from the foregoing. Examples of certain sequencing methods are described hereafter.

A sequence analysis apparatus or sequence analysis component(s) includes an apparatus, and one or more components used in conjunction with such apparatus, that can be used by a person of ordinary skill to determine a nucleotide sequence resulting from processes described herein (e.g., linear and/or exponential amplification products). Examples of sequencing platforms include, without limitation, the 454 platform (Roche) (Margulies, M. et al. 2005 Nature 437, 376-380), Illumina Genomic Analyzer (or Solexa platform) or SOLID System (Applied Biosystems; see PCT patent application publications WO 06/084132 entitled “Reagents, Methods, and Libraries For Bead-Based Sequencing” and W007/121,489 entitled “Reagents, Methods, and Libraries for Gel-Free Bead-Based Sequencing”), the Helicos True Single Molecule DNA sequencing technology (Harris TD et al. 2008 Science, 320, 106-109), the single molecule, real-time (SMRT™) technology of Pacific Biosciences, and nanopore sequencing (Soni G V and Meller A. 2007 Clin Chem 53: 1996-2001), Ion semiconductor sequencing (Ion Torrent Systems, Inc, San Francisco, CA), or DNA nanoball sequencing (Complete Genomics, Mountain View, CA), VisiGen Biotechnologies approach (Invitrogen) and polony sequencing. Such platforms allow sequencing of many nucleic acid molecules isolated from a specimen at high orders of multiplexing in a parallel manner (Dear Brief Funct Genomic Proteomic 2003; 1: 397-416; Haimovich, Methods, challenges, and promise of next-generation sequencing in cancer biology. Yale J Biol Med. 2011 December; 84(4):439-46). These non-Sanger-based sequencing technologies are sometimes referred to as NextGen sequencing, NGS, next-generation sequencing, next generation sequencing, and variations thereof. Typically they allow much higher throughput than the traditional Sanger approach. See Schuster, Next-generation sequencing transforms today's biology, Nature Methods 5:16-18 (2008); Metzker, Sequencing technologies—the next generation. Nat Rev Genet. 2010 January; 11(1):31-46. These platforms can allow sequencing of clonally expanded or non-amplified single molecules of nucleic acid fragments. Certain platforms involve, for example, sequencing by ligation of dye-modified probes (including cyclic ligation and cleavage), pyrosequencing, and single-molecule sequencing. Nucleotide sequence species, amplification nucleic acid species and detectable products generated there from can be analyzed by such sequence analysis platforms. Next-generation sequencing can be used in the methods of the invention, e.g., to determine mutations, copy number, or expression levels, as appropriate. The methods can be used to perform whole genome sequencing or sequencing of specific sequences of interest, such as a gene of interest or a fragment thereof.

Sequencing by ligation is a nucleic acid sequencing method that relies on the sensitivity of DNA ligase to base-pairing mismatch. DNA ligase joins together ends of DNA that are correctly base paired. Combining the ability of DNA ligase to join together only correctly base paired DNA ends, with mixed pools of fluorescently labeled oligonucleotides or primers, enables sequence determination by fluorescence detection. Longer sequence reads may be obtained by including primers containing cleavable linkages that can be cleaved after label identification. Cleavage at the linker removes the label and regenerates the 5′ phosphate on the end of the ligated primer, preparing the primer for another round of ligation. In some embodiments primers may be labeled with more than one fluorescent label, e.g., at least 1, 2, 3, 4, or 5 fluorescent labels.

Sequencing by ligation generally involves the following steps. Clonal bead populations can be prepared in emulsion microreactors containing target nucleic acid template sequences, amplification reaction components, beads and primers. After amplification, templates are denatured and bead enrichment is performed to separate beads with extended templates from undesired beads (e.g., beads with no extended templates). The template on the selected beads undergoes a 3′ modification to allow covalent bonding to the slide, and modified beads can be deposited onto a glass slide. Deposition chambers offer the ability to segment a slide into one, four or eight chambers during the bead loading process. For sequence analysis, primers hybridize to the adapter sequence. A set of four color dye-labeled probes competes for ligation to the sequencing primer. Specificity of probe ligation is achieved by interrogating every 4th and 5th base during the ligation series. Five to seven rounds of ligation, detection and cleavage record the color at every 5th position with the number of rounds determined by the type of library used. Following each round of ligation, a new complimentary primer offset by one base in the 5′ direction is laid down for another series of ligations. Primer reset and ligation rounds (5-7 ligation cycles per round) are repeated sequentially five times to generate 25-35 base pairs of sequence for a single tag. With mate-paired sequencing, this process is repeated for a second tag.

Pyrosequencing is a nucleic acid sequencing method based on sequencing by synthesis, which relies on detection of a pyrophosphate released on nucleotide incorporation. Generally, sequencing by synthesis involves synthesizing, one nucleotide at a time, a DNA strand complimentary to the strand whose sequence is being sought. Target nucleic acids may be immobilized to a solid support, hybridized with a sequencing primer, incubated with DNA polymerase, ATP sulfurylase, luciferase, apyrase, adenosine 5′ phosphosulfate and luciferin. Nucleotide solutions are sequentially added and removed. Correct incorporation of a nucleotide releases a pyrophosphate, which interacts with ATP sulfurylase and produces ATP in the presence of adenosine 5′ phosphosulfate, fueling the luciferin reaction, which produces a chemiluminescent signal allowing sequence determination. The amount of light generated is proportional to the number of bases added. Accordingly, the sequence downstream of the sequencing primer can be determined. An illustrative system for pyrosequencing involves the following steps: ligating an adaptor nucleic acid to a nucleic acid under investigation and hybridizing the resulting nucleic acid to a bead; amplifying a nucleotide sequence in an emulsion; sorting beads using a picoliter multiwell solid support; and sequencing amplified nucleotide sequences by pyrosequencing methodology (e.g., Nakano et al., “Single-molecule PCR using water-in-oil emulsion;” Journal of Biotechnology 102: 117-124 (2003)).

Certain single-molecule sequencing embodiments are based on the principal of sequencing by synthesis, and use single-pair Fluorescence Resonance Energy Transfer (single pair FRET) as a mechanism by which photons are emitted as a result of successful nucleotide incorporation. The emitted photons often are detected using intensified or high sensitivity cooled charge-couple-devices in conjunction with total internal reflection microscopy (TIRM). Photons are only emitted when the introduced reaction solution contains the correct nucleotide for incorporation into the growing nucleic acid chain that is synthesized as a result of the sequencing process. In FRET based single-molecule sequencing, energy is transferred between two fluorescent dyes, sometimes polymethine cyanine dyes Cy3 and Cy5, through long-range dipole interactions. The donor is excited at its specific excitation wavelength and the excited state energy is transferred, non-radiatively to the acceptor dye, which in turn becomes excited. The acceptor dye eventually returns to the ground state by radiative emission of a photon. The two dyes used in the energy transfer process represent the “single pair” in single pair FRET. Cy3 often is used as the donor fluorophore and often is incorporated as the first labeled nucleotide. Cy5 often is used as the acceptor fluorophore and is used as the nucleotide label for successive nucleotide additions after incorporation of a first Cy3 labeled nucleotide. The fluorophores generally are within 10 nanometers of each for energy transfer to occur successfully.

An example of a system that can be used based on single-molecule sequencing generally involves hybridizing a primer to a target nucleic acid sequence to generate a complex; associating the complex with a solid phase; iteratively extending the primer by a nucleotide tagged with a fluorescent molecule; and capturing an image of fluorescence resonance energy transfer signals after each iteration (e.g., U.S. Pat. No. 7,169,314; Braslavsky et al., PNAS 100(7): 3960-3964 (2003)). Such a system can be used to directly sequence amplification products (linearly or exponentially amplified products) generated by processes described herein. In some embodiments the amplification products can be hybridized to a primer that contains sequences complementary to immobilized capture sequences present on a solid support, a bead or glass slide for example. Hybridization of the primer-amplification product complexes with the immobilized capture sequences, immobilizes amplification products to solid supports for single pair FRET based sequencing by synthesis. The primer often is fluorescent, so that an initial reference image of the surface of the slide with immobilized nucleic acids can be generated. The initial reference image is useful for determining locations at which true nucleotide incorporation is occurring. Fluorescence signals detected in array locations not initially identified in the “primer only” reference image are discarded as non-specific fluorescence. Following immobilization of the primer-amplification product complexes, the bound nucleic acids often are sequenced in parallel by the iterative steps of, a) polymerase extension in the presence of one fluorescently labeled nucleotide, b) detection of fluorescence using appropriate microscopy, TIRM for example, c) removal of fluorescent nucleotide, and d) return to step a with a different fluorescently labeled nucleotide.

In some embodiments, nucleotide sequencing may be by solid phase single nucleotide sequencing methods and processes. Solid phase single nucleotide sequencing methods involve contacting target nucleic acid and solid support under conditions in which a single molecule of sample nucleic acid hybridizes to a single molecule of a solid support. Such conditions can include providing the solid support molecules and a single molecule of target nucleic acid in a “microreactor.” Such conditions also can include providing a mixture in which the target nucleic acid molecule can hybridize to solid phase nucleic acid on the solid support. Single nucleotide sequencing methods useful in the embodiments described herein are described in U.S. Provisional Patent Application Ser. No. 61/021,871 filed Jan. 17, 2008.

In certain embodiments, nanopore sequencing detection methods include (a) contacting a target nucleic acid for sequencing (“base nucleic acid,” e.g., linked probe molecule) with sequence-specific detectors, under conditions in which the detectors specifically hybridize to substantially complementary subsequences of the base nucleic acid; (b) detecting signals from the detectors and (c) determining the sequence of the base nucleic acid according to the signals detected. In certain embodiments, the detectors hybridized to the base nucleic acid are disassociated from the base nucleic acid (e.g., sequentially dissociated) when the detectors interfere with a nanopore structure as the base nucleic acid passes through a pore, and the detectors disassociated from the base sequence are detected. In some embodiments, a detector disassociated from a base nucleic acid emits a detectable signal, and the detector hybridized to the base nucleic acid emits a different detectable signal or no detectable signal. In certain embodiments, nucleotides in a nucleic acid (e.g., linked probe molecule) are substituted with specific nucleotide sequences corresponding to specific nucleotides (“nucleotide representatives”), thereby giving rise to an expanded nucleic acid (e.g., U.S. Pat. No. 6,723,513), and the detectors hybridize to the nucleotide representatives in the expanded nucleic acid, which serves as a base nucleic acid. In such embodiments, nucleotide representatives may be arranged in a binary or higher order arrangement (e.g., Soni and Meller, Clinical Chemistry 53(11): 1996-2001 (2007)). In some embodiments, a nucleic acid is not expanded, does not give rise to an expanded nucleic acid, and directly serves a base nucleic acid (e.g., a linked probe molecule serves as a non-expanded base nucleic acid), and detectors are directly contacted with the base nucleic acid. For example, a first detector may hybridize to a first subsequence and a second detector may hybridize to a second subsequence, where the first detector and second detector each have detectable labels that can be distinguished from one another, and where the signals from the first detector and second detector can be distinguished from one another when the detectors are disassociated from the base nucleic acid. In certain embodiments, detectors include a region that hybridizes to the base nucleic acid (e.g., two regions), which can be about 3 to about 100 nucleotides in length (e.g., about 4, 5, 6, 7, 8, 9, 10, 11, 12, 13, 14, 15, 16, 17, 18, 19, 20, 25, 30, 35, 40, 50, 55, 60, 65, 70, 75, 80, 85, 90, or 95 nucleotides in length). A detector also may include one or more regions of nucleotides that do not hybridize to the base nucleic acid. In some embodiments, a detector is a molecular beacon. A detector often comprises one or more detectable labels independently selected from those described herein. Each detectable label can be detected by any convenient detection process capable of detecting a signal generated by each label (e.g., magnetic, electric, chemical, optical and the like). For example, a CD camera can be used to detect signals from one or more distinguishable quantum dots linked to a detector.

In certain sequence analysis embodiments, reads may be used to construct a larger nucleotide sequence, which can be facilitated by identifying overlapping sequences in different reads and by using identification sequences in the reads. Such sequence analysis methods and software for constructing larger sequences from reads are known to the person of ordinary skill (e.g., Venter et al., Science 291: 1304-1351 (2001)). Specific reads, partial nucleotide sequence constructs, and full nucleotide sequence constructs may be compared between nucleotide sequences within a sample nucleic acid (i.e., internal comparison) or may be compared with a reference sequence (i.e., reference comparison) in certain sequence analysis embodiments. Internal comparisons can be performed in situations where a sample nucleic acid is prepared from multiple samples or from a single sample source that contains sequence variations. Reference comparisons sometimes are performed when a reference nucleotide sequence is known and an objective is to determine whether a sample nucleic acid contains a nucleotide sequence that is substantially similar or the same, or different, than a reference nucleotide sequence. Sequence analysis can be facilitated by the use of sequence analysis apparatus and components described above.

Primer extension polymorphism detection methods, also referred to herein as “microsequencing” methods, typically are carried out by hybridizing a complementary oligonucleotide to a nucleic acid carrying the polymorphic site. In these methods, the oligonucleotide typically hybridizes adjacent to the polymorphic site. The term “adjacent” as used in reference to “microsequencing” methods, refers to the 3′ end of the extension oligonucleotide being sometimes 1 nucleotide from the 5′ end of the polymorphic site, often 2 or 3, and at times 4, 5, 6, 7, 8, 9, or 10 nucleotides from the 5′ end of the polymorphic site, in the nucleic acid when the extension oligonucleotide is hybridized to the nucleic acid. The extension oligonucleotide then is extended by one or more nucleotides, often 1, 2, or 3 nucleotides, and the number and/or type of nucleotides that are added to the extension oligonucleotide determine which polymorphic variant or variants are present. Oligonucleotide extension methods are disclosed, for example, in U.S. Pat. Nos. 4,656,127; 4,851,331; 5,679,524; 5,834,189; 5,876,934; 5,908,755; 5,912,118; 5,976,802; 5,981,186; 6,004,744; 6,013,431; 6,017,702; 6,046,005; 6,087,095; 6,210,891; and WO 01/20039. The extension products can be detected in any manner, such as by fluorescence methods (see, e.g., Chen & Kwok, Nucleic Acids Research 25: 347-353 (1997) and Chen et al., Proc. Natl. Acad. Sci. USA 94/20: 10756-10761 (1997)) or by mass spectrometric methods (e.g., MALDI-TOF mass spectrometry) and other methods described herein. Oligonucleotide extension methods using mass spectrometry are described, for example, in U.S. Pat. Nos. 5,547,835; 5,605,798; 5,691,141; 5,849,542; 5,869,242; 5,928,906; 6,043,031; 6,194,144; and 6,258,538.

Microsequencing detection methods often incorporate an amplification process that proceeds the extension step. The amplification process typically amplifies a region from a nucleic acid sample that comprises the polymorphic site. Amplification can be carried out using methods described above, or for example using a pair of oligonucleotide primers in a polymerase chain reaction (PCR), in which one oligonucleotide primer typically is complementary to a region 3′ of the polymorphism and the other typically is complementary to a region 5′ of the polymorphism. A PCR primer pair may be used in methods disclosed in U.S. Pat. Nos. 4,683,195; 4,683,202, 4,965,188; 5,656,493; 5,998,143; 6,140,054; WO 01/27327; and WO 01/27329 for example. PCR primer pairs may also be used in any commercially available machines that perform PCR, such as any of the GeneAmp™ Systems available from Applied Biosystems.

Other appropriate sequencing methods include multiplex polony sequencing (as described in Shendure et al., Accurate Multiplex Polony Sequencing of an Evolved Bacterial Genome, Sciencexpress, Aug. 4, 2105, pg 1, incorporated herein by reference), which employs immobilized microbeads, and sequencing in microfabricated picoliter reactors (as described in Margulies et al. Genome Sequencing in Microfabricated High-Density Picoliter Reactors, Nature, August 2005, incorporated herein by reference).

Whole genome sequencing may also be used for discriminating alleles of RNA transcripts, in some embodiments. Examples of whole genome sequencing methods include, but are not limited to, nanopore-based sequencing methods, sequencing by synthesis and sequencing by ligation, as described above.

Nucleic acid variants can also be detected using standard electrophoretic techniques. Although the detection step can sometimes be preceded by an amplification step, amplification is not required in the embodiments described herein. Examples of methods for detection and quantification of a nucleic acid using electrophoretic techniques can be found in the art. A non-limiting example comprises running a sample (e.g., mixed nucleic acid sample isolated from maternal serum, or amplification nucleic acid species, for example) in an agarose or polyacrylamide gel. The gel may be labeled (e.g., stained) with ethidium bromide (see, Sambrook and Russell, Molecular Cloning: A Laboratory Manual 3d ed., 2001). The presence of a band of the same size as the standard control is an indication of the presence of a target nucleic acid sequence, the amount of which may then be compared to the control based on the intensity of the band, thus detecting and quantifying the target sequence of interest. In some embodiments, restriction enzymes capable of distinguishing between maternal and paternal alleles may be used to detect and quantify target nucleic acid species. In certain embodiments, oligonucleotide probes specific to a sequence of interest are used to detect the presence of the target sequence of interest. The oligonucleotides can also be used to indicate the amount of the target nucleic acid molecules in comparison to the standard control, based on the intensity of signal imparted by the probe.

Sequence-specific probe hybridization can be used to detect a particular nucleic acid in a mixture or mixed population comprising other species of nucleic acids. Under sufficiently stringent hybridization conditions, the probes hybridize specifically only to substantially complementary sequences. The stringency of the hybridization conditions can be relaxed to tolerate varying amounts of sequence mismatch. A number of hybridization formats are known in the art, which include but are not limited to, solution phase, solid phase, or mixed phase hybridization assays. The following articles provide an overview of the various hybridization assay formats: Singer et al., Biotechniques 4:230, 1986; Haase et al., Methods in Virology, pp. 189-226, 1984; Wilkinson, In situ Hybridization, Wilkinson ed., IRL Press, Oxford University Press, Oxford; and Hames and Higgins eds., Nucleic Acid Hybridization: A Practical Approach, IRL Press, 1987.

Hybridization complexes can be detected by techniques known in the art. Nucleic acid probes capable of specifically hybridizing to a target nucleic acid (e.g., mRNA or DNA) can be labeled by any suitable method, and the labeled probe used to detect the presence of hybridized nucleic acids. One commonly used method of detection is autoradiography, using probes labeled with ³H, ¹²⁵I, ³⁵S, ¹⁴C, ³²P ³³P or the like. The choice of radioactive isotope depends on research preferences due to ease of synthesis, stability, and half-lives of the selected isotopes. Other labels include compounds (e.g., biotin and digoxigenin), which bind to antiligands or antibodies labeled with fluorophores, chemiluminescent agents, and enzymes. In some embodiments, probes can be conjugated directly with labels such as fluorophores, chemiluminescent agents or enzymes. The choice of label depends on sensitivity required, ease of conjugation with the probe, stability requirements, and available instrumentation.

In embodiments, fragment analysis (referred to herein as “FA”) methods are used for molecular profiling. Fragment analysis (FA) includes techniques such as restriction fragment length polymorphism (RFLP) and/or (amplified fragment length polymorphism). If a nucleotide variant in the target DNA corresponding to the one or more genes results in the elimination or creation of a restriction enzyme recognition site, then digestion of the target DNA with that particular restriction enzyme will generate an altered restriction fragment length pattern. Thus, a detected RFLP or AFLP will indicate the presence of a particular nucleotide variant.

Terminal restriction fragment length polymorphism (TRFLP) works by PCR amplification of DNA using primer pairs that have been labeled with fluorescent tags. The PCR products are digested using RFLP enzymes and the resulting patterns are visualized using a DNA sequencer. The results are analyzed either by counting and comparing bands or peaks in the TRFLP profile, or by comparing bands from one or more TRFLP runs in a database.

The sequence changes directly involved with an RFLP can also be analyzed more quickly by PCR. Amplification can be directed across the altered restriction site, and the products digested with the restriction enzyme. This method has been called Cleaved Amplified Polymorphic Sequence (CAPS). Alternatively, the amplified segment can be analyzed by Allele specific oligonucleotide (ASO) probes, a process that is sometimes assessed using a Dot blot.

A variation on AFLP is cDNA-AFLP, which can be used to quantify differences in gene expression levels.

Another useful approach is the single-stranded conformation polymorphism assay (SSCA), which is based on the altered mobility of a single-stranded target DNA spanning the nucleotide variant of interest. A single nucleotide change in the target sequence can result in different intramolecular base pairing pattern, and thus different secondary structure of the single-stranded DNA, which can be detected in a non-denaturing gel. See Orita et al., Proc. Natl. Acad. Sci. USA, 86:2776-2770 (1989). Denaturing gel-based techniques such as clamped denaturing gel electrophoresis (CDGE) and denaturing gradient gel electrophoresis (DGGE) detect differences in migration rates of mutant sequences as compared to wild-type sequences in denaturing gel. See Miller et al., Biotechniques, 5:1016-24 (1999); Sheffield et al., Am. J. Hum, Genet., 49:699-706 (1991); Wartell et al., Nucleic Acids Res., 18:2699-2705 (1990); and Sheffield et al., Proc. Natl. Acad. Sci. USA, 86:232-236 (1989). In addition, the double-strand conformation analysis (DSCA) can also be useful in the present invention. See Arguello et al., Nat. Genet., 18:192-194 (1998).

The presence or absence of a nucleotide variant at a particular locus in the one or more genes of an individual can also be detected using the amplification refractory mutation system (ARMS) technique. See e.g., European Patent No. 0,332,435; Newton et al., Nucleic Acids Res., 17:2503-2515 (1989); Fox et al., Br. J. Cancer, 77:1267-1274 (1998); Robertson et al., Eur. Respir. J., 12:477-482 (1998). In the ARMS method, a primer is synthesized matching the nucleotide sequence immediately 5′ upstream from the locus being tested except that the 3′-end nucleotide which corresponds to the nucleotide at the locus is a predetermined nucleotide. For example, the 3′-end nucleotide can be the same as that in the mutated locus. The primer can be of any suitable length so long as it hybridizes to the target DNA under stringent conditions only when its 3′-end nucleotide matches the nucleotide at the locus being tested. Preferably the primer has at least 12 nucleotides, more preferably from about 18 to 50 nucleotides. If the individual tested has a mutation at the locus and the nucleotide therein matches the 3′-end nucleotide of the primer, then the primer can be further extended upon hybridizing to the target DNA template, and the primer can initiate a PCR amplification reaction in conjunction with another suitable PCR primer. In contrast, if the nucleotide at the locus is of wild type, then primer extension cannot be achieved. Various forms of ARMS techniques developed in the past few years can be used. See e.g., Gibson et al., Clin. Chem. 43:1336-1341 (1997).

Similar to the ARMS technique is the mini sequencing or single nucleotide primer extension method, which is based on the incorporation of a single nucleotide. An oligonucleotide primer matching the nucleotide sequence immediately 5′ to the locus being tested is hybridized to the target DNA, mRNA or miRNA in the presence of labeled dideoxyribonucleotides. A labeled nucleotide is incorporated or linked to the primer only when the dideoxyribonucleotides matches the nucleotide at the variant locus being detected. Thus, the identity of the nucleotide at the variant locus can be revealed based on the detection label attached to the incorporated dideoxyribonucleotides. See Syvanen et al., Genomics, 8:684-692 (1990); Shumaker et al., Hum. Mutat., 7:346-354 (1996); Chen et al., Genome Res., 10:549-547 (2000).

Another set of techniques useful in the present invention is the so-called “oligonucleotide ligation assay” (OLA) in which differentiation between a wild-type locus and a mutation is based on the ability of two oligonucleotides to anneal adjacent to each other on the target DNA molecule allowing the two oligonucleotides joined together by a DNA ligase. See Landergren et al., Science, 241:1077-1080 (1988); Chen et al, Genome Res., 8:549-556 (1998); Iannone et al., Cytometry, 39:131-140 (2000). Thus, for example, to detect a single-nucleotide mutation at a particular locus in the one or more genes, two oligonucleotides can be synthesized, one having the sequence just 5′ upstream from the locus with its 3′ end nucleotide being identical to the nucleotide in the variant locus of the particular gene, the other having a nucleotide sequence matching the sequence immediately 3′ downstream from the locus in the gene. The oligonucleotides can be labeled for the purpose of detection. Upon hybridizing to the target gene under a stringent condition, the two oligonucleotides are subject to ligation in the presence of a suitable ligase. The ligation of the two oligonucleotides would indicate that the target DNA has a nucleotide variant at the locus being detected.

Detection of small genetic variations can also be accomplished by a variety of hybridization-based approaches. Allele-specific oligonucleotides are most useful. See Conner et al., Proc. Natl. Acad. Sci. USA, 80:278-282 (1983); Saiki et al, Proc. Natl. Acad. Sci. USA, 86:6230-6234 (1989). Oligonucleotide probes (allele-specific) hybridizing specifically to a gene allele having a particular gene variant at a particular locus but not to other alleles can be designed by methods known in the art. The probes can have a length of, e.g., from 10 to about 50 nucleotide bases. The target DNA and the oligonucleotide probe can be contacted with each other under conditions sufficiently stringent such that the nucleotide variant can be distinguished from the wild-type gene based on the presence or absence of hybridization. The probe can be labeled to provide detection signals. Alternatively, the allele-specific oligonucleotide probe can be used as a PCR amplification primer in an “allele-specific PCR” and the presence or absence of a PCR product of the expected length would indicate the presence or absence of a particular nucleotide variant.

Other useful hybridization-based techniques allow two single-stranded nucleic acids annealed together even in the presence of mismatch due to nucleotide substitution, insertion or deletion. The mismatch can then be detected using various techniques. For example, the annealed duplexes can be subject to electrophoresis. The mismatched duplexes can be detected based on their electrophoretic mobility that is different from the perfectly matched duplexes. See Cariello, Human Genetics, 42:726 (1988). Alternatively, in an RNase protection assay, a RNA probe can be prepared spanning the nucleotide variant site to be detected and having a detection marker. See Giunta et al., Diagn. Mol. Path., 5:265-270 (1996); Finkelstein et al., Genomics, 7:167-172 (1990); Kinszler et al., Science 251:1366-1370 (1991). The RNA probe can be hybridized to the target DNA or mRNA forming a heteroduplex that is then subject to the ribonuclease RNase A digestion. RNase A digests the RNA probe in the heteroduplex only at the site of mismatch. The digestion can be determined on a denaturing electrophoresis gel based on size variations. In addition, mismatches can also be detected by chemical cleavage methods known in the art. See e.g., Roberts et al., Nucleic Acids Res., 25:3377-3378 (1997).

In the mutS assay, a probe can be prepared matching the gene sequence surrounding the locus at which the presence or absence of a mutation is to be detected, except that a predetermined nucleotide is used at the variant locus. Upon annealing the probe to the target DNA to form a duplex, the E. coli mutS protein is contacted with the duplex. Since the mutS protein binds only to heteroduplex sequences containing a nucleotide mismatch, the binding of the mutS protein will be indicative of the presence of a mutation. See Modrich et al., Ann. Rev. Genet., 25:229-253 (1991).

A great variety of improvements and variations have been developed in the art on the basis of the above-described basic techniques which can be useful in detecting mutations or nucleotide variants in the present invention. For example, the “sunrise probes” or “molecular beacons” use the fluorescence resonance energy transfer (FRET) property and give rise to high sensitivity. See Wolf et al., Proc. Nat. Acad. Sci. USA, 85:8790-8794 (1988). Typically, a probe spanning the nucleotide locus to be detected are designed into a hairpin-shaped structure and labeled with a quenching fluorophore at one end and a reporter fluorophore at the other end. In its natural state, the fluorescence from the reporter fluorophore is quenched by the quenching fluorophore due to the proximity of one fluorophore to the other. Upon hybridization of the probe to the target DNA, the 5′ end is separated apart from the 3′-end and thus fluorescence signal is regenerated. See Nazarenko et al., Nucleic Acids Res., 25:2516-2521 (1997); Rychlik et al., Nucleic Acids Res., 17:8543-8551 (1989); Sharkey et al., Bio/Technology 12:506-509 (1994); Tyagi et al., Nat. Biotechnol., 14:303-308 (1996); Tyagi et al., Nat. Biotechnol., 16:49-53 (1998). The homo-tag assisted non-dimer system (HANDS) can be used in combination with the molecular beacon methods to suppress primer-dimer accumulation. See Brownie et al., Nucleic Acids Res., 25:3235-3241 (1997).

Dye-labeled oligonucleotide ligation assay is a FRET-based method, which combines the OLA assay and PCR. See Chen et al., Genome Res. 8:549-556 (1998). TaqMan is another FRET-based method for detecting nucleotide variants. A TaqMan probe can be oligonucleotides designed to have the nucleotide sequence of the gene spanning the variant locus of interest and to differentially hybridize with different alleles. The two ends of the probe are labeled with a quenching fluorophore and a reporter fluorophore, respectively. The TaqMan probe is incorporated into a PCR reaction for the amplification of a target gene region containing the locus of interest using Taq polymerase. As Taq polymerase exhibits 5′-3′ exonuclease activity but has no 3′-5′ exonuclease activity, if the TaqMan probe is annealed to the target DNA template, the 5′-end of the TaqMan probe will be degraded by Taq polymerase during the PCR reaction thus separating the reporting fluorophore from the quenching fluorophore and releasing fluorescence signals. See Holland et al., Proc. Natl. Acad. Sci. USA, 88:7276-7280 (1991); Kalinina et al., Nucleic Acids Res., 25:1999-2004 (1997); Whitcombe et al., Clin. Chem., 44:918-923 (1998).

In addition, the detection in the present invention can also employ a chemiluminescence-based technique. For example, an oligonucleotide probe can be designed to hybridize to either the wild-type or a variant gene locus but not both. The probe is labeled with a highly chemiluminescent acridinium ester. Hydrolysis of the acridinium ester destroys chemiluminescence. The hybridization of the probe to the target DNA prevents the hydrolysis of the acridinium ester. Therefore, the presence or absence of a particular mutation in the target DNA is determined by measuring chemiluminescence changes. See Nelson et al., Nucleic Acids Res., 24:4998-5003 (1996).

The detection of genetic variation in the gene in accordance with the present invention can also be based on the “base excision sequence scanning” (BESS) technique. The BESS method is a PCR-based mutation scanning method. BESS T-Scan and BESS G-Tracker are generated which are analogous to T and G ladders of dideoxy sequencing. Mutations are detected by comparing the sequence of normal and mutant DNA. See, e.g., Hawkins et al., Electrophoresis, 20:1171-1176 (1999).

Mass spectrometry can be used for molecular profiling according to the invention. See Graber et al., Curr. Opin. Biotechnol., 9:14-18 (1998). For example, in the primer oligo base extension (PROBE™) method, a target nucleic acid is immobilized to a solid-phase support. A primer is annealed to the target immediately 5′ upstream from the locus to be analyzed. Primer extension is carried out in the presence of a selected mixture of deoxyribonucleotides and dideoxyribonucleotides. The resulting mixture of newly extended primers is then analyzed by MALDI-TOF. See e.g., Monforte et al., Nat. Med., 3:360-362 (1997).

In addition, the microchip or microarray technologies are also applicable to the detection method of the present invention. Essentially, in microchips, a large number of different oligonucleotide probes are immobilized in an array on a substrate or carrier, e.g., a silicon chip or glass slide. Target nucleic acid sequences to be analyzed can be contacted with the immobilized oligonucleotide probes on the microchip. See Lipshutz et al., Biotechniques, 19:442-447 (1995); Chee et al., Science, 274:610-614 (1996); Kozal et al., Nat. Med. 2:753-759 (1996); Hacia et al., Nat. Genet., 14:441-447 (1996); Saiki et al., Proc. Natl. Acad. Sci. USA, 86:6230-6234 (1989); Gingeras et al., Genome Res., 8:435-448 (1998). Alternatively, the multiple target nucleic acid sequences to be studied are fixed onto a substrate and an array of probes is contacted with the immobilized target sequences. See Drmanac et al., Nat. Biotechnol., 16:54-58 (1998). Numerous microchip technologies have been developed incorporating one or more of the above described techniques for detecting mutations. The microchip technologies combined with computerized analysis tools allow fast screening in a large scale. The adaptation of the microchip technologies to the present invention will be apparent to a person of skill in the art apprised of the present disclosure. See, e.g., U.S. Pat. No. 5,925,525 to Fodor et al; Wilgenbus et al., J. Mol. Med., 77:761-786 (1999); Graber et al., Curr. Opin. Biotechnol., 9:14-18 (1998); Hacia et al., Nat. Genet., 14:441-447 (1996); Shoemaker et al., Nat. Genet., 14:450-456 (1996); DeRisi et al., Nat. Genet., 14:457-460 (1996); Chee et al., Nat. Genet., 14:610-614 (1996); Lockhart et al., Nat. Genet., 14:675-680 (1996); Drobyshev et al., Gene, 188:45-52 (1997).

As is apparent from the above survey of the suitable detection techniques, it may or may not be necessary to amplify the target DNA, i.e., the gene, cDNA, mRNA, miRNA, or a portion thereof to increase the number of target DNA molecule, depending on the detection techniques used. For example, most PCR-based techniques combine the amplification of a portion of the target and the detection of the mutations. PCR amplification is well known in the art and is disclosed in U.S. Pat. Nos. 4,683,195 and 4,800,159, both which are incorporated herein by reference. For non-PCR-based detection techniques, if necessary, the amplification can be achieved by, e.g., in vivo plasmid multiplication, or by purifying the target DNA from a large amount of tissue or cell samples. See generally, Sambrook et al., Molecular Cloning: A Laboratory Manual, 2^{nd} ed., Cold Spring Harbor Laboratory, Cold Spring Harbor, N.Y., 1989. However, even with scarce samples, many sensitive techniques have been developed in which small genetic variations such as single-nucleotide substitutions can be detected without having to amplify the target DNA in the sample. For example, techniques have been developed that amplify the signal as opposed to the target DNA by, e.g., employing branched DNA or dendrimers that can hybridize to the target DNA. The branched or dendrimer DNAs provide multiple hybridization sites for hybridization probes to attach thereto thus amplifying the detection signals. See Detmer et al., J. Clin. Microbiol., 34:901-907 (1996); Collins et al., Nucleic Acids Res., 25:2979-2984 (1997); Horn et al., Nucleic Acids Res., 25:4835-4841 (1997); Horn et al., Nucleic Acids Res., 25:4842-4849 (1997); Nilsen et al., J. Theor. Biol., 187:273-284 (1997).

The Invader™ assay is another technique for detecting single nucleotide variations that can be used for molecular profiling according to the invention. The Invader™ assay uses a novel linear signal amplification technology that improves upon the long turnaround times required of the typical PCR DNA sequenced-based analysis. See Cooksey et al., Antimicrobial Agents and Chemotherapy 44:1296-1301 (2000). This assay is based on cleavage of a unique secondary structure formed between two overlapping oligonucleotides that hybridize to the target sequence of interest to form a “flap.” Each “flap” then generates thousands of signals per hour. Thus, the results of this technique can be easily read, and the methods do not require exponential amplification of the DNA target. The Invader™ system uses two short DNA probes, which are hybridized to a DNA target. The structure formed by the hybridization event is recognized by a special cleavase enzyme that cuts one of the probes to release a short DNA “flap.” Each released “flap” then binds to a fluorescently-labeled probe to form another cleavage structure. When the cleavase enzyme cuts the labeled probe, the probe emits a detectable fluorescence signal. See e.g. Lyamichev et al., Nat. Biotechnol., 17:292-296 (1999).

The rolling circle method is another method that avoids exponential amplification. Lizardi et al., Nature Genetics, 19:225-232 (1998) (which is incorporated herein by reference). For example, Sniper™, a commercial embodiment of this method, is a sensitive, high-throughput SNP scoring system designed for the accurate fluorescent detection of specific variants. For each nucleotide variant, two linear, allele-specific probes are designed. The two allele-specific probes are identical with the exception of the 3′-base, which is varied to complement the variant site. In the first stage of the assay, target DNA is denatured and then hybridized with a pair of single, allele-specific, open-circle oligonucleotide probes. When the 3′-base exactly complements the target DNA, ligation of the probe will preferentially occur. Subsequent detection of the circularized oligonucleotide probes is by rolling circle amplification, whereupon the amplified probe products are detected by fluorescence. See Clark and Pickering, Life Science News 6, 2000, Amersham Pharmacia Biotech (2000).

A number of other techniques that avoid amplification all together include, e.g., surface-enhanced resonance Raman scattering (SERRS), fluorescence correlation spectroscopy, and single-molecule electrophoresis. In SERRS, a chromophore-nucleic acid conjugate is absorbed onto colloidal silver and is irradiated with laser light at a resonant frequency of the chromophore. See Graham et al., Anal. Chem., 69:4703-4707 (1997). The fluorescence correlation spectroscopy is based on the spatio-temporal correlations among fluctuating light signals and trapping single molecules in an electric field. See Eigen et al., Proc. Natl. Acad. Sci. USA, 91:5740-5747 (1994). In single-molecule electrophoresis, the electrophoretic velocity of a fluorescently tagged nucleic acid is determined by measuring the time required for the molecule to travel a predetermined distance between two laser beams. See Castro et al., Anal. Chem., 67:3181-3186 (1995).

In addition, the allele-specific oligonucleotides (ASO) can also be used in in situ hybridization using tissues or cells as samples. The oligonucleotide probes which can hybridize differentially with the wild-type gene sequence or the gene sequence harboring a mutation may be labeled with radioactive isotopes, fluorescence, or other detectable markers. In situ hybridization techniques are well known in the art and their adaptation to the present invention for detecting the presence or absence of a nucleotide variant in the one or more gene of a particular individual should be apparent to a skilled artisan apprised of this disclosure.

Accordingly, the presence or absence of one or more genes nucleotide variant or amino acid variant in an individual can be determined using any of the detection methods described above.

Typically, once the presence or absence of one or more gene nucleotide variants or amino acid variants is determined, physicians or genetic counselors or patients or other researchers may be informed of the result. Specifically the result can be cast in a transmittable form that can be communicated or transmitted to other researchers or physicians or genetic counselors or patients. Such a form can vary and can be tangible or intangible. The result with regard to the presence or absence of a nucleotide variant of the present invention in the individual tested can be embodied in descriptive statements, diagrams, photographs, charts, images or any other visual forms. For example, images of gel electrophoresis of PCR products can be used in explaining the results. Diagrams showing where a variant occurs in an individual's gene are also useful in indicating the testing results. The statements and visual forms can be recorded on a tangible media such as papers, computer readable media such as floppy disks, compact disks, etc., or on an intangible media, e.g., an electronic media in the form of email or website on internet or intranet. In addition, the result with regard to the presence or absence of a nucleotide variant or amino acid variant in the individual tested can also be recorded in a sound form and transmitted through any suitable media, e.g., analog or digital cable lines, fiber optic cables, etc., via telephone, facsimile, wireless mobile phone, internet phone and the like.

Thus, the information and data on a test result can be produced anywhere in the world and transmitted to a different location. For example, when a genotyping assay is conducted offshore, the information and data on a test result may be generated and cast in a transmittable form as described above. The test result in a transmittable form thus can be imported into the U.S. Accordingly, the present invention also encompasses a method for producing a transmittable form of information on the genotype of the two or more suspected cancer samples from an individual. The method comprises the steps of (1) determining the genotype of the DNA from the samples according to methods of the present invention; and (2) embodying the result of the determining step in a transmittable form. The transmittable form is the product of the production method.

### In Situ Hybridization

In situ hybridization assays are well known and are generally described in Angerer et al., Methods Enzymol. 152:649-660 (1987). In an in situ hybridization assay, cells, e.g., from a biopsy, are fixed to a solid support, typically a glass slide. If DNA is to be probed, the cells are denatured with heat or alkali. The cells are then contacted with a hybridization solution at a moderate temperature to permit annealing of specific probes that are labeled. The probes are preferably labeled, e.g., with radioisotopes or fluorescent reporters, or enzymatically. FISH (fluorescence in situ hybridization) uses fluorescent probes that bind to only those parts of a sequence with which they show a high degree of sequence similarity. CISH (chromogenic in situ hybridization) uses conventional peroxidase or alkaline phosphatase reactions visualized under a standard bright-field microscope.

In situ hybridization can be used to detect specific gene sequences in tissue sections or cell preparations by hybridizing the complementary strand of a nucleotide probe to the sequence of interest. Fluorescent in situ hybridization (FISH) uses a fluorescent probe to increase the sensitivity of in situ hybridization.

FISH is a cytogenetic technique used to detect and localize specific polynucleotide sequences in cells. For example, FISH can be used to detect DNA sequences on chromosomes. FISH can also be used to detect and localize specific RNAs, e.g., mRNAs, within tissue samples. In FISH uses fluorescent probes that bind to specific nucleotide sequences to which they show a high degree of sequence similarity. Fluorescence microscopy can be used to find out whether and where the fluorescent probes are bound. In addition to detecting specific nucleotide sequences, e.g., translocations, fusion, breaks, duplications and other chromosomal abnormalities, FISH can help define the spatial-temporal patterns of specific gene copy number and/or gene expression within cells and tissues.

Various types of FISH probes can be used to detect chromosome translocations. Dual color, single fusion probes can be useful in detecting cells possessing a specific chromosomal translocation. The DNA probe hybridization targets are located on one side of each of the two genetic breakpoints. “Extra signal” probes can reduce the frequency of normal cells exhibiting an abnormal FISH pattern due to the random co-localization of probe signals in a normal nucleus. One large probe spans one breakpoint, while the other probe flanks the breakpoint on the other gene. Dual color, break apart probes are useful in cases where there may be multiple translocation partners associated with a known genetic breakpoint. This labeling scheme features two differently colored probes that hybridize to targets on opposite sides of a breakpoint in one gene. Dual color, dual fusion probes can reduce the number of normal nuclei exhibiting abnormal signal patterns. The probe offers advantages in detecting low levels of nuclei possessing a simple balanced translocation. Large probes span two breakpoints on different chromosomes. Such probes are available as Vysis probes from Abbott Laboratories, Abbott Park, IL.

CISH, or chromogenic in situ hybridization, is a process in which a labeled complementary DNA or RNA strand is used to localize a specific DNA or RNA sequence in a tissue specimen. CISH methodology can be used to evaluate gene amplification, gene deletion, chromosome translocation, and chromosome number. CISH can use conventional enzymatic detection methodology, e.g., horseradish peroxidase or alkaline phosphatase reactions, visualized under a standard bright-field microscope. In a common embodiment, a probe that recognizes the sequence of interest is contacted with a sample. An antibody or other binding agent that recognizes the probe, e.g., via a label carried by the probe, can be used to target an enzymatic detection system to the site of the probe. In some systems, the antibody can recognize the label of a FISH probe, thereby allowing a sample to be analyzed using both FISH and CISH detection. CISH can be used to evaluate nucleic acids in multiple settings, e.g., formalin-fixed, paraffin-embedded (FFPE) tissue, blood or bone marrow smear, metaphase chromosome spread, and/or fixed cells. In an embodiment, CISH is performed following the methodology in the SPoT-Light® HER2 CISH Kit available from Life Technologies (Carlsbad, CA) or similar CISH products available from Life Technologies. The SPoT-Light® HER2 CISH Kit itself is FDA approved for in vitro diagnostics and can be used for molecular profiling of HER2. CISH can be used in similar applications as FISH. Thus, one of skill will appreciate that reference to molecular profiling using FISH herein can be performed using CISH, unless otherwise specified.

Silver-enhanced in situ hybridization (SISH) is similar to CISH, but with SISH the signal appears as a black coloration due to silver precipitation instead of the chromogen precipitates of CISH.

Modifications of the in situ hybridization techniques can be used for molecular profiling according to the invention. Such modifications comprise simultaneous detection of multiple targets, e.g., Dual ISH, Dual color CISH, bright field double in situ hybridization (BDISH). See e.g., the FDA approved INFORM HER2 Dual ISH DNA Probe Cocktail kit from Ventana Medical Systems, Inc. (Tucson, AZ); DuoCISH™, a dual color CISH kit developed by Dako Denmark A/S (Denmark).

Comparative Genomic Hybridization (CGH) comprises a molecular cytogenetic method of screening tumor samples for genetic changes showing characteristic patterns for copy number changes at chromosomal and subchromosomal levels. Alterations in patterns can be classified as DNA gains and losses. CGH employs the kinetics of in situ hybridization to compare the copy numbers of different DNA or RNA sequences from a sample, or the copy numbers of different DNA or RNA sequences in one sample to the copy numbers of the substantially identical sequences in another sample. In many useful applications of CGH, the DNA or RNA is isolated from a subject cell or cell population. The comparisons can be qualitative or quantitative. Procedures are described that permit determination of the absolute copy numbers of DNA sequences throughout the genome of a cell or cell population if the absolute copy number is known or determined for one or several sequences. The different sequences are discriminated from each other by the different locations of their binding sites when hybridized to a reference genome, usually metaphase chromosomes but in certain cases interphase nuclei. The copy number information originates from comparisons of the intensities of the hybridization signals among the different locations on the reference genome. The methods, techniques and applications of CGH are known, such as described in U.S. Pat. No. 6,335,167, and in U.S. App. Ser. No. 60/804,818, the relevant parts of which are herein incorporated by reference.

In an embodiment, CGH used to compare nucleic acids between diseased and healthy tissues. The method comprises isolating DNA from disease tissues (e.g., tumors) and reference tissues (e.g., healthy tissue) and labeling each with a different “color” or fluor. The two samples are mixed and hybridized to normal metaphase chromosomes. In the case of array or matrix CGH, the hybridization mixing is done on a slide with thousands of DNA probes. A variety of detection system can be used that basically determine the color ratio along the chromosomes to determine DNA regions that might be gained or lost in the diseased samples as compared to the reference.

### Molecular Profiling for Treatment Selection

The methods of the invention provide a candidate treatment selection for a subject in need thereof. Molecular profiling can be used to identify one or more candidate therapeutic agents for an individual suffering from a condition in which one or more of the biomarkers disclosed herein are targets for treatment. For example, the method can identify one or more chemotherapy treatments for a cancer. In an aspect, the invention provides a method comprising: performing at least one molecular profiling technique on at least one biomarker. Any relevant biomarker can be assessed using one or more of the molecular profiling techniques described herein or known in the art. The marker need only have some direct or indirect association with a treatment to be useful. Any relevant molecular profiling technique can be performed, such as those disclosed here. These can include without limitation, protein and nucleic acid analysis techniques. Protein analysis techniques include, by way of non-limiting examples, immunoassays, immunohistochemistry, and mass spectrometry. Nucleic acid analysis techniques include, by way of non-limiting examples, amplification, polymerase chain amplification, hybridization, microarrays, in situ hybridization, sequencing, dye-terminator sequencing, next generation sequencing, pyrosequencing, and restriction fragment analysis.

Molecular profiling may comprise the profiling of at least one gene (or gene product) for each assay technique that is performed. Different numbers of genes can be assayed with different techniques. Any marker disclosed herein that is associated directly or indirectly with a target therapeutic can be assessed. For example, any “druggable target” comprising a target that can be modulated with a therapeutic agent such as a small molecule or binding agent such as an antibody, is a candidate for inclusion in the molecular profiling methods of the invention. The target can also be indirectly drug associated, such as a component of a biological pathway that is affected by the associated drug. The molecular profiling can be based on either the gene, e.g., DNA sequence, and/or gene product, e.g., mRNA or protein. Such nucleic acid and/or polypeptide can be profiled as applicable as to presence or absence, level or amount, activity, mutation, sequence, haplotype, rearrangement, copy number, or other measurable characteristic. In some embodiments, a single gene and/or one or more corresponding gene products is assayed by more than one molecular profiling technique. A gene or gene product (also referred to herein as “marker” or “biomarker”), e.g., an mRNA or protein, is assessed using applicable techniques (e.g., to assess DNA, RNA, protein), including without limitation ISH, gene expression, IHC, sequencing or immunoassay. Therefore, any of the markers disclosed herein can be assayed by a single molecular profiling technique or by multiple methods disclosed herein (e.g., a single marker is profiled by one or more of IHC, ISH, sequencing, microarray, etc.). In some embodiments, at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95 or at least about 100 genes or gene products are profiled by at least one technique, a plurality of techniques, or using any desired combination of ISH, IHC, gene expression, gene copy, and sequencing. In some embodiments, at least about 100, 200, 300, 400, 500, 600, 700, 800, 900, 1000, 2000, 3000, 4000, 5000, 6000, 7000, 8000, 9000, 10,000, 11,000, 12,000, 13,000, 14,000, 15,000, 16,000, 17,000, 18,000, 19,000, 20,000, 21,000, 22,000, 23,000, 24,000, 25,000, 26,000, 27,000, 28,000, 29,000, 30,000, 31,000, 32,000, 33,000, 34,000, 35,000, 36,000, 37,000, 38,000, 39,000, 40,000, 41,000, 42,000, 43,000, 44,000, 45,000, 46,000, 47,000, 48,000, 49,000, or at least 50,000 genes or gene products are profiled using various techniques. The number of markers assayed can depend on the technique used. For example, microarray and massively parallel sequencing lend themselves to high throughput analysis. Because molecular profiling queries molecular characteristics of the tumor itself, this approach provides information on therapies that might not otherwise be considered based on the lineage of the tumor.

In some embodiments, a sample from a subject in need thereof is profiled using methods which include but are not limited to IHC analysis, gene expression analysis, ISH analysis, and/or sequencing analysis (such as by PCR, RT-PCR, pyrosequencing, NGS) for one or more of the following: ABCC1, ABCG2, ACE2, ADA, ADH1C, ADH4, AGT, AR, AREG, ASNS, BCL2, BCRP, BDCA1, beta III tubulin, BIRC5, B-RAF, BRCA1, BRCA2, CA2, caveolin, CD20, CD25, CD33, CD52, CDA, CDKN2A, CDKN1A, CDKN1B, CDK2, CDW52, CES2, CK 14, CK 17, CK 5/6, c-KIT, c-Met, c-Myc, COX-2, Cyclin D1, DCK, DHFR, DNMT1, DNMT3A, DNMT3B, E-Cadherin, ECGF1, EGFR, EML4-ALK fusion, EPHA2, Epiregulin, ER, ERBR2, ERCC1, ERCC3, EREG, ESR1, FLT1, folate receptor, FOLR1, FOLR2, FSHB, FSHPRH1, FSHR, FYN, GART, GNA11, GNAQ, GNRH1, GNRHR1, GSTP1, HCK, HDAC1, hENT-1, Her2/Neu, HGF, HIF1A, HIG1, HSP90, HSP90AA1, HSPCA, IGF-1R, IGFRBP, IGFRBP3, IGFRBP4, IGFRBP5, IL13RA1, IL2RA, KDR, Ki67, KIT, K-RAS, LCK, LTB, Lymphotoxin Beta Receptor, LYN, MET, MGMT, MLH1, MMR, MRP1, MS4A1, MSH2, MSH5, Myc, NFKB1, NFKB2, NFKBIA, NRAS, ODC1, OGFR, p16, p21, p27, p53, p95, PARP-1, PDGFC, PDGFR, PDGFRA, PDGFRB, PGP, PGR, PI3K, POLA, POLA1, PPARG, PPARGC1, PR, PTEN, PTGS2, PTPN12, RAF1, RARA, ROS1, RRM1, RRM2, RRM2B, RXRB, RXRG, SIK2, SPARC, SRC, SSTR1, SSTR2, SSTR3, SSTR4, SSTR5, Survivin, TK1, TLE3, TNF, TOP1, TOP2A, TOP2B, TS, TUBB3, TXN, TXNRD1, TYMS, VDR, VEGF, VEGFA, VEGFC, VHL, YES1, ZAP70.

As understood by those of skill i. the art, genes and proteins have developed a number of alternative names in the scientific literature. Listing of gen aliases and descriptions used herein can be found using a variety of online databases, including GeneCards®, HUGO Gene Nonmenclature, EnErez Gene, UniProtKB/Swiss-Prot, UniProtKB/TrEMBL, OMIM, GeneLoc, and Ensembl. For example, gene symbols and names used herein can correspond to those approved by HUGO, and protein names can be those recommended by UniProtKB/Swiss-Prot, In the specification, where a protein name indicates a precursor, the mature protein is also implied. Throughout the application, gene and protein symbols may be used interchangeably and the meaning can be derived from context, e.g., ISH or NGS can be used to analyze nucleic acids whereas THC is used to analyze protein.

The choice of genes and gene products to be assessed to provide molecular profiles of the invention can be updated over time as new treatments and new drug targets are identified. For example, once the expression or mutation of a biomarker is correlated with a treatment option, it can be assessed by molecular profiling. One of skill will appreciate that such molecular profiling is not limited to those techniques disclosed herein but comprises any methodology conventional for assessing nucleic acid or protein levels, sequence information, or both. The methods of the invention can also take advantage of any improvements to current methods or new molecular profiling techniques developed in the future. In some embodiments, a gene or gene product is assessed by a single molecular profiling technique. In other embodiments, a gene and/or gene product is assessed by multiple molecular profiling techniques. In a non-limiting example, a gene sequence can be assayed by one or more of NGS, ISH and pyrosequencing analysis, the mRNA gene product can be assayed by one or more of NGS, RT-PCR and microarray, and the protein gene product can be assayed by one or more of IHC and immunoassay. One of skill will appreciate that any combination of biomarkers and molecular profiling techniques that will benefit disease treatment are contemplated by the invention.

Genes and gene products that are known to play a role in cancer and can be assayed by any of the molecular profiling techniques of the invention include without limitation those listed in any of International Patent Publications WO/2007/137187 (Int'l Appl. No. PCT/US2007/069286), published Nov. 29, 2007; WO/2010/045318 (Int'l Appl. No. PCT/US2009/060630), published Apr. 22, 2010; WO/2010/093465 (Int'l Appl. No. PCT/US2010/000407), published Aug. 19, 2010; WO/2012/170715 (Int'l Appl. No. PCT/US2012/041393), published Dec. 13, 2012; WO/2014/089241 (Int'l Appl. No. PCT/US2013/073184), published Jun. 12, 2014; WO/2011/056688 (Int'l Appl. No. PCT/US2010/054366), published May 12, 2011; WO/2012/092336 (Int'l Appl. No. PCT/US2011/067527), published Jul. 5, 2012; WO/2015/116868 (Int'l Appl. No. PCT/US2015/013618), published Aug. 6, 2015; WO/2017/053915 (Int'l Appl. No. PCT/US2016/053614), published Mar. 30, 2017; and WO/2016/141169 (Int'l Appl. No. PCT/US2016/020657), published Sep. 9, 2016; each of which publications is incorporated by reference herein in its entirety.

Mutation profiling can be determined by sequencing, including Sanger sequencing, array sequencing, pyrosequencing, NextGen sequencing, etc. Sequence analysis may reveal that genes harbor activating mutations so that drugs that inhibit activity are indicated for treatment. Alternately, sequence analysis may reveal that genes harbor mutations that inhibit or eliminate activity, thereby indicating treatment for compensating therapies. In some embodiments, sequence analysis comprises that of exon 9 and 11 of c-KIT. Sequencing may also be performed on EGFR-kinase domain exons 18, 19, 20, and 21. Mutations, amplifications or misregulations of EGFR or its family members are implicated in about 30% of all epithelial cancers. Sequencing can also be performed on PI3K, encoded by the PIK3CA gene. This gene is a found mutated in many cancers. Sequencing analysis can also comprise assessing mutations in one or more ABCC1, ABCG2, ADA, AR, ASNS, BCL2, BIRC5, BRCA1, BRCA2, CD33, CD52, CDA, CES2, DCK, DHFR, DNMT1, DNMT3A, DNMT3B, ECGF1, EGFR, EPHA2, ERBB2, ERCC1, ERCC3, ESR1, FLT1, FOLR2, FYN, GART, GNRH1, GSTP1, HCK, HDAC1, HIF1A, HSP90AA1, IGFBP3, IGFBP4, IGFBP5, IL2RA, KDR, KIT, LCK, LYN, MET, MGMT, MLH1, MS4A1, MSH2, NFKB1, NFKB2, NFKBIA, NRAS, OGFR, PARP1, PDGFC, PDGFRA, PDGFRB, PGP, PGR, POLA1, PTEN, PTGS2, PTPN12, RAF1, RARA, RRM1, RRM2, RRM2B, RXRB, RXRG, SIK2, SPARC, SRC, SSTR1, SSTR2, SSTR3, SSTR4, SSTR5, TK1, TNF, TOP1, TOP2A, TOP2B, TXNRD1, TYMS, VDR, VEGFA, VHL, YES1, and ZAP70. One or more of the following genes can also be assessed by sequence analysis: ALK, EML4, hENT-1, IGF-1R, HSP90AA1, MMR, p16, p21, p27, PARP-1, PI3K and TLE3. The genes and/or gene products used for mutation or sequence analysis can be at least 1, 2, 3, 4, 5, 6, 7, 8, 9, 10, 15, 20, 25, 30, 40, 50, 60, 70, 80, 90, 100, 200, 300, 400, 500 or all of the genes and/or gene products listed in any of Tables 4-12, e.g., in any of Tables 5-10, or in any of Tables 7-10.

In embodiments, the methods of the invention are used detect gene fusions, such as those listed in any of International Patent Publications WO/2007/137187 (Int'l Appl. No. PCT/US2007/069286), published Nov. 29, 2007; WO/2010/045318 (Int'l Appl. No. PCT/US2009/060630), published Apr. 22, 2010; WO/2010/093465 (Int'l Appl. No. PCT/US2010/000407), published Aug. 19, 2010; WO/2012/170715 (Int'l Appl. No. PCT/US2012/041393), published Dec. 13, 2012; WO/2014/089241 (Int'l Appl. No. PCT/US2013/073184), published Jun. 12, 2014; WO/2011/056688 (Int'l Appl. No. PCT/US2010/054366), published May 12, 2011; WO/2012/092336 (Int'l Appl. No. PCT/US2011/067527), published Jul. 5, 2012; WO/2015/116868 (Int'l Appl. No. PCT/US2015/013618), published Aug. 6, 2015; WO/2017/053915 (Int'l Appl. No. PCT/US2016/053614), published Mar. 30, 2017; and WO/2016/141169 (Int'l Appl. No. PCT/US2016/020657), published Sep. 9, 2016; each of which publications is incorporated by reference herein in its entirety. A fusion gene is a hybrid gene created by the juxtaposition of two previously separate genes. This can occur by chromosomal translocation or inversion, deletion or via trans-splicing. The resulting fusion gene can cause abnormal temporal and spatial expression of genes, leading to abnormal expression of cell growth factors, angiogenesis factors, tumor promoters or other factors contributing to the neoplastic transformation of the cell and the creation of a tumor. For example, such fusion genes can be oncogenic due to the juxtaposition of: 1) a strong promoter region of one gene next to the coding region of a cell growth factor, tumor promoter or other gene promoting oncogenesis leading to elevated gene expression, or 2) due to the fusion of coding regions of two different genes, giving rise to a chimeric gene and thus a chimeric protein with abnormal activity. Fusion genes are characteristic of many cancers. Once a therapeutic intervention is associated with a fusion, the presence of that fusion in any type of cancer identifies the therapeutic intervention as a candidate therapy for treating the cancer.

The presence of fusion genes can be used to guide therapeutic selection. For example, the BCR-ABL gene fusion is a characteristic molecular aberration in ˜90% of chronic myelogenous leukemia (CML) and in a subset of acute leukemias (Kurzrock et al., Annals of Internal Medicine 2003; 138:819-830). The BCR-ABL results from a translocation between chromosomes 9 and 22, commonly referred to as the Philadelphia chromosome or Philadelphia translocation. The translocation brings together the 5′ region of the BCR gene and the 3′ region of ABL1, generating a chimeric BCR-ABL1 gene, which encodes a protein with constitutively active tyrosine kinase activity (Mittleman et al., Nature Reviews Cancer 2007; 7:233-245). The aberrant tyrosine kinase activity leads to de-regulated cell signaling, cell growth and cell survival, apoptosis resistance and growth factor independence, all of which contribute to the pathophysiology of leukemia (Kurzrock et al., Annals of Internal Medicine 2003; 138:819-830). Patients with the Philadelphia chromosome are treated with imatinib and other targeted therapies. Imatinib binds to the site of the constitutive tyrosine kinase activity of the fusion protein and prevents its activity. Imatinib treatment has led to molecular responses (disappearance of BCR-ABL+blood cells) and improved progression-free survival in BCR-ABL+CML patients (Kantarjian et al., Clinical Cancer Research 2007; 13:1089-1097).

Another fusion gene, IGH-MYC, is a defining feature of ˜80% of Burkitt's lymphoma (Ferry et al. Oncologist 2006; 11:375-83). The causal event for this is a translocation between chromosomes 8 and 14, bringing the c-Myc oncogene adjacent to the strong promoter of the immunoglobulin heavy chain gene, causing c-myc overexpression (Mittleman et al., Nature Reviews Cancer 2007; 7:233-245). The c-myc rearrangement is a pivotal event in lymphomagenesis as it results in a perpetually proliferative state. It has wide ranging effects on progression through the cell cycle, cellular differentiation, apoptosis, and cell adhesion (Ferry et al. Oncologist 2006; 11:375-83).

A number of recurrent fusion genes have been catalogued in the Mittleman database (cgap.nci.nih.gov/Chromosomes/Mitelman). The gene fusions can be used to characterize neoplasms and cancers and guide therapy using the subject methods described herein. For example, TMPRSS2-ERG, TMPRSS2-ETV and SLC45A3-ELK4 fusions can be detected to characterize prostate cancer; and ETV6-NTRK3 and ODZ4-NRG1 can be used to characterize breast cancer. The EML4-ALK, RLF-MYCL1, TGF-ALK, or CD74-ROS1 fusions can be used to characterize a lung cancer. The ACSL3-ETV1, C150RF21-ETV1, FLJ35294-ETV1, HERV-ETV1, TMPRSS2-ERG, TMPRSS2-ETV1/4/5, TMPRSS2-ETV4/5, SLC5A3-ERG, SLC5A3-ETV1, SLC5A3-ETV5 or KLK2-ETV4 fusions can be used to characterize a prostate cancer. The GOPC-ROS1 fusion can be used to characterize a brain cancer. The CHCHD7-PLAG1, CTNNB1-PLAG1, FHIT-HMGA2, HMGA2-NFIB, LIFR-PLAG1, or TCEAl-PLAG1 fusions can be used to characterize a head and neck cancer. The ALPHA-TFEB, NONO-TFE3, PRCC-TFE3, SFPQ-TFE3, CLTC-TFE3, or MALAT1-TFEB fusions can be used to characterize a renal cell carcinoma (RCC). The AKAP9-BRAF, CCDC6-RET, ERC1-RETM, GOLGA5-RET, HOOK3-RET, HRH4-RET, KTN1-RET, NCOA4-RET, PCM1-RET, PRKARA1A-RET, RFG-RET, RFG9-RET, Ria-RET, TGF-NTRK1, TPM3-NTRK1, TPM3-TPR, TPR-MET, TPR-NTRK1, TRIM24-RET, TRIM27-RET or TRIM33-RET fusions can be used to characterize a thyroid cancer and/or papillary thyroid carcinoma; and the PAX8-PPARy fusion can be analyzed to characterize a follicular thyroid cancer. Fusions that are associated with hematological malignancies include without limitation TTL-ETV6, CDK6-MLL, CDK6-TLX3, ETV6-FLT3, ETV6-RUNX1, ETV6-TTL, MLL-AFF1, MLL-AFF3, MLL-AFF4, MLL-GAS7, TCBA1-ETV6, TCF3-PBX1 or TCF3-TFPT, which are characteristic of acute lymphocytic leukemia (ALL); BCL11B-TLX3, IL2-TNFRFS17, NUP214-ABL1, NUP98-CCDC28A, TAL1-STIL, or ETV6-ABL2, which are characteristic of T-cell acute lymphocytic leukemia (T-ALL); ATIC-ALK, KIAA1618-ALK, MSN-ALK, MYH9-ALK, NPM1-ALK, TGF-ALK or TPM3-ALK, which are characteristic of anaplastic large cell lymphoma (ALCL); BCR-ABL1, BCR-JAK2, ETV6-EVI1, ETV6-MN1 or ETV6-TCBA1, characteristic of chronic myelogenous leukemia (CML); CBFB-MYH11, CHIC2-ETV6, ETV6-ABL1, ETV6-ABL2, ETV6-ARNT, ETV6-CDX2, ETV6-HLXB9, ETV6-PER1, MEF2D-DAZAP1, AML-AFF1, MLL-ARHGAP26, MLL-ARHGEF12, MLL-CASC5, MLL-CBL,MLL-CREBBP, MLL-DAB21P, MLL-ELL, MLL-EP300, MLL-EPS15, MLL-FNBP1, MLL-FOXO3A, MLL-GMPS, MLL-GPHN, MLL-MLLT1, MLL-MLLT11, MLL-MLLT3, MLL-MLLT6, MLL-MYO1F, MLL-PICALM, MLL-SEPT2, MLL-SEPT6, MLL-SORBS2, MYST3-SORBS2, MYST-CREBBP, NPM1-MLF1, NUP98-HOXA13, PRDM16-EVI1, RABEP1-PDGFRB, RUNX1-EVI1, RUNX1-MDS1, RUNX1-RPL22, RUNX1-RUNX1T1, RUNX1-SH3D19, RUNX1-USP42, RUNX1-YTHDF2, RUNX1-ZNF687, or TAF15-ZNF-384, which are characteristic of acute myeloid leukemia (AML); CCND1-FSTL3, which is characteristic of chronic lymphocytic leukemia (CLL); BCL3-MYC, MYC-BTGl, BCL7A-MYC, BRWD3-ARHGAP20 or BTGl-MYC, which are characteristic of B-cell chronic lymphocytic leukemia (B-CLL); CITTA-BCL6, CLTC-ALK, IL21R-BCL6, PIM1-BCL6, TFCR-BCL6, IKZF1-BCL6 or SEC31A-ALK, which are characteristic of diffuse large B-cell lymphomas (DLBCL); FLIP1-PDGFRA, FLT3-ETV6, KIAA1509-PDGFRA, PDE4DIP-PDGFRB, NIN-PDGFRB, TP53BP1-PDGFRB, or TPM3-PDGFRB, which are characteristic of hyper eosinophilia/chronic eosinophilia; and IGH-MYC or LCP1-BCL6, which are characteristic of Burkitt's lymphoma. One of skill will understand that additional fusions, including those yet to be identified to date, can be used to guide treatment once their presence is associated with a therapeutic intervention.

The fusion genes and gene products can be detected using one or more techniques described herein. In some embodiments, the sequence of the gene or corresponding mRNA is determined, e.g., using Sanger sequencing, NGS, pyrosequencing, DNA microarrays, etc. Chromosomal abnormalities can be assessed using ISH, NGS or PCR techniques, among others. For example, a break apart probe can be used for ISH detection of ALK fusions such as EML4-ALK, KIF5B-ALK and/or TFG-ALK. As an alternate, PCR can be used to amplify the fusion product, wherein amplification or lack thereof indicates the presence or absence of the fusion, respectively. mRNA can be sequenced, e.g., using NGS to detect such fusions. See, e.g., Table 9 or Table 12 herein. In some embodiments, the fusion protein fusion is detected. Appropriate methods for protein analysis include without limitation mass spectroscopy, electrophoresis (e.g., 2D gel electrophoresis or SDS-PAGE) or antibody related techniques, including immunoassay, protein array or immunohistochemistry. The techniques can be combined. As a non-limiting example, indication of an ALK fusion by NGS can be confirmed by ISH or ALK expression using IHC, or vice versa.

### Treatment Selection

The systems and methods allow identification of one or more therapeutic targets whose projected efficacy can be linked to therapeutic efficacy, ultimately based on the molecular profiling. Illustrative schemes for using molecular profiling to identify a treatment regime are provided throughout, e.g., in Tables 2-3, Table 11, FIGS. 2, 26A-F and 28, each of which is described in further detail herein. Additional schemes are described in International Patent Publications WO/2007/137187 (Int'l Appl. No. PCT/US2007/069286), published Nov. 29, 2007; WO/2010/045318 (Int'l Appl. No. PCT/US2009/060630), published Apr. 22, 2010; WO/2010/093465 (Int'l Appl. No. PCT/US2010/000407), published Aug. 19, 2010; WO/2012/170715 (Int'l Appl. No. PCT/US2012/041393), published Dec. 13, 2012; WO/2014/089241 (Int'l Appl. No. PCT/US2013/073184), published Jun. 12, 2014; WO/2011/056688 (Int'l Appl. No. PCT/US2010/054366), published May 12, 2011; WO/2012/092336 (Int'l Appl. No. PCT/US2011/067527), published Jul. 5, 2012; WO/2015/116868 (Int'l Appl. No. PCT/US2015/013618), published Aug. 6, 2015; WO/2017/053915 (Int'l Appl. No. PCT/US2016/053614), published Mar. 30, 2017; and WO/2016/141169 (Int'l Appl. No. PCT/US2016/020657), published Sep. 9, 2016; each of which publications is incorporated by reference herein in its entirety. The invention comprises use of molecular profiling results to suggest associations with treatment responses. In an embodiment, the appropriate biomarkers for molecular profiling are selected on the basis of the subject's tumor type. These suggested biomarkers can be used to modify a default list of biomarkers. In other embodiments, the molecular profiling is independent of the source material. In some embodiments, rules are used to provide the suggested chemotherapy treatments based on the molecular profiling test results. In an embodiment, the rules are generated from abstracts of the peer reviewed clinical oncology literature. Expert opinion rules can be used but are optional. In an embodiment, clinical citations are assessed for their relevance to the methods of the invention using a hierarchy derived from the evidence grading system used by the United States Preventive Services Taskforce. The “best evidence” can be used as the basis for a rule. The simplest rules are constructed in the format of “if biomarker positive then treatment option one, else treatment option two.” Treatment options comprise no treatment with a specific drug, treatment with a specific drug or treatment with a combination of drugs. In some embodiments, more complex rules are constructed that involve the interaction of two or more biomarkers. In such cases, the more complex interactions are typically supported by clinical studies that analyze the interaction between the biomarkers included in the rule. Finally, a report can be generated that describes the association of the chemotherapy response and the biomarker and a summary statement of the best evidence supporting the treatments selected. Ultimately, the treating physician will decide on the best course of treatment.

As a non-limiting example, molecular profiling might reveal that the EGFR gene is amplified or overexpressed, thus indicating selection of a treatment that can block EGFR activity, such as the monoclonal antibody inhibitors cetuximab and panitumumab, or small molecule kinase inhibitors effective in patients with activating mutations in EGFR such as gefitinib, erlotinib, and lapatinib. Other anti-EGFR monoclonal antibodies in clinical development include zalutumumab, nimotuzumab, and matuzumab. The candidate treatment selected can depend on the setting revealed by molecular profiling. For example, kinase inhibitors are often prescribed with EGFR is found to have activating mutations. Continuing with the illustrative embodiment, molecular profiling may also reveal that some or all of these treatments are likely to be less effective. For example, patients taking gefitinib or erlotinib eventually develop drug resistance mutations in EGFR. Accordingly, the presence of a drug resistance mutation would contraindicate selection of the small molecule kinase inhibitors. One of skill will appreciate that this example can be expanded to guide the selection of other candidate treatments that act against genes or gene products whose differential expression is revealed by molecular profiling. Similarly, candidate agents known to be effective against diseased cells carrying certain nucleic acid variants can be selected if molecular profiling reveals such variants.

As another example, consider the drug imatinib, currently marketed by Novartis as Gleevec in the US in the form of imatinib mesylate. Imatinib is a 2-phenylaminopyrimidine derivative that functions as a specific inhibitor of a number of tyrosine kinase enzymes. It occupies the tyrosine kinase active site, leading to a decrease in kinase activity. Imatinib has been shown to block the activity of Abelson cytoplasmic tyrosine kinase (ABL), c-Kit and the platelet-derived growth factor receptor (PDGFR). Thus, imatinib can be indicated as a candidate therapeutic for a cancer determined by molecular profiling to overexpress ABL, c-KIT or PDGFR. Imatinib can be indicated as a candidate therapeutic for a cancer determined by molecular profiling to have mutations in ABL, c-KIT or PDGFR that alter their activity, e.g., constitutive kinase activity of ABLs caused by the BCR-ABL mutation. As an inhibitor of PDGFR, imatinib mesylate appears to have utility in the treatment of a variety of dermatological diseases.

Cancer therapies that can be identified as candidate treatments by the methods of the invention include without limitation those listed in any of International Patent Publications WO/2007/137187 (Int'l Appl. No. PCT/US2007/069286), published Nov. 29, 2007; WO/2010/045318 (Int'l Appl. No. PCT/US2009/060630), published Apr. 22, 2010; WO/2010/093465 (Int'l Appl. No. PCT/US2010/000407), published Aug. 19, 2010; WO/2012/170715 (Int'l Appl. No. PCT/US2012/041393), published Dec. 13, 2012; WO/2014/089241 (Int'l Appl. No. PCT/US2013/073184), published Jun. 12, 2014; WO/2011/056688 (Int'l Appl. No. PCT/US2010/054366), published May 12, 2011; WO/2012/092336 (Int'l Appl. No. PCT/US2011/067527), published Jul. 5, 2012; WO/2015/116868 (Int'l Appl. No. PCT/US2015/013618), published Aug. 6, 2015; WO/2017/053915 (Int'l Appl. No. PCT/US2016/053614), published Mar. 30, 2017; and WO/2016/141169 (Int'l Appl. No. PCT/US2016/020657), published Sep. 9, 2016; each of which publications is incorporated by reference herein in its entirety. The candidate treatments can be any of those in Table 11 herein.

### Rules Engine

In some embodiments, a database is created that maps treatments and molecular profiling results. The treatment information can include the projected efficacy of a therapeutic agent against cells having certain attributes that can be measured by molecular profiling. The molecular profiling can include differential expression or mutations in certain genes, proteins, or other biological molecules of interest. Through the mapping, the results of the molecular profiling can be compared against the database to select treatments. The database can include both positive and negative mappings between treatments and molecular profiling results. In some embodiments, the mapping is created by reviewing the literature for links between biological agents and therapeutic agents. For example, a journal article, patent publication or patent application publication, scientific presentation, etc can be reviewed for potential mappings. The mapping can include results of in vivo, e.g., animal studies or clinical trials, or in vitro experiments, e.g., cell culture. Any mappings that are found can be entered into the database, e.g., cytotoxic effects of a therapeutic agent against cells expressing a gene or protein. In this manner, the database can be continuously updated. It will be appreciated that the methods of the invention are updated as well.

The rules can be generated by evidence-based literature review. Biomarker research continues to provide a better understanding of the clinical behavior and biology of cancer. This body of literature can be maintained in an up-to-date data repository incorporating recent clinical studies relevant to treatment options and potential clinical outcomes. The studies can be ranked so that only those with the strongest or most reliable evidence are selected for rules generation. For example, the rules generation can employ the grading system from the current methods of the U.S. Preventive Services Task Force. The literature evidence can be reviewed and evaluated based on the strength of clinical evidence supporting associations between biomarkers and treatments in the literature study. This process can be performed by a staff of scientists, physicians and other skilled reviewers. The process can also be automated in whole or in part by using language search and heuristics to identify relevant literature. The rules can be generated by a review of a plurality of literature references, e.g., tens, hundreds, thousands or more literature articles.

In another aspect, the invention provides a method of generating a set of evidence-based associations, comprising: (a) searching one or more literature database by a computer using an evidence-based medicine search filter to identify articles comprising a gene or gene product thereof, a disease, and one or more therapeutic agent; (b) filtering the articles identified in (a) to compile evidence-based associations comprising the expected benefit and/or the expected lack of benefit of the one or more therapeutic agent for treating the disease given the status of the gene or gene product; (c) adding the evidence-based associations compiled in (b) to the set of evidence-based associations; and (d) repeating steps (a)-(c) for an additional gene or gene product thereof. The status of the gene can include one or more assessments as described herein which relate to a biological state, e.g., one or more of an expression level, a copy number, and a mutation. The genes or gene products thereof can be one or more genes or gene products thereof selected from Table 2, Tables 6-9 or Tables 12-15. For example, the method can be repeated for at least 1, e.g., at least 1, 2, 3, 4, 5, 6, 7, 8, 9, 10, 15, 20, 25, 30, 40, 50, 60, 70, 80, 90, 100, 200, 300, 400, 500, 600 or at least 700 of the genes or gene products thereof in Table 2, Tables 6-9 or Tables 12-15. The disease can be a disease described here, e.g., in embodiment the disease comprises a cancer. The one or more literature database can be selected from the group consisting of the National Library of Medicine's (NLM's) MEDLINE™ database of citations, a patent literature database, and a combination thereof.

Evidence-based medicine (EBM) or evidence-based practice (EBP) aims to apply the best available evidence gained from the scientific method to clinical decision making. This approach assesses the strength of evidence of the risks and benefits of treatments (including lack of treatment) and diagnostic tests. Evidence quality can be assessed based on the source type (from meta-analyses and systematic reviews of double-blind, placebo-controlled clinical trials at the top end, down to conventional wisdom at the bottom), as well as other factors including statistical validity, clinical relevance, currency, and peer-review acceptance. Evidence-based medicine filters are searches that have been developed to facilitate searches in specific areas of clinical medicine related to evidence-based medicine (diagnosis, etiology, meta-analysis, prognosis and therapy). They are designed to retrieve high quality evidence from published studies appropriate to decision-making. The evidence-based medicine filter used in the invention can be selected from the group consisting of a generic evidence-based medicine filter, a McMaster University optimal search strategy evidence-based medicine filter, a University of York statistically developed search evidence-based medicine filter, and a University of California San Francisco systemic review evidence-based medicine filter. See e.g., US Patent Publication 20080215570; Shojania and Bero. Taking advantage of the explosion of systematic reviews: an efficient MEDLINE search strategy. Eff Clin Pract. 2001 Jul-Aug; 4(4):157-62; Ingui and Rogers. Searching for clinical prediction rules in MEDLINE. J Am Med Inform Assoc. 2001 Jul-Aug; 8(4):391-7; Haynes et al., Optimal search strategies for retrieving scientifically strong studies of treatment from Medline: analytical survey. BMJ. 2005 May 21;330(7501):1179; Wilczynski and Haynes. Consistency and accuracy of indexing systematic review articles and meta-analyses in medline. Health Info Libr J. 2009 September; 26(3):203-10; which references are incorporated by reference herein in their entirety. A generic filter can be a customized filter based on an algorithm to identify the desired references from the one or more literature database. For example, the method can use one or more approach as described in U.S. Pat. No. 5,168,533 to Kato et al., U.S. Pat. No. 6,886,010 to Kostoff, or US Patent Application Publication No. 20040064438 to Kostoff; which references are incorporated by reference herein in their entirety.

The further filtering of articles identified by the evidence-based medicine filter can be performed using a computer, by one or more expert user, or combination thereof. The one or more expert can be a trained scientist or physician. In embodiments, the set of evidence-based associations comprise one or more of the rules in Table 11 herein. The set of evidence-based associations include without limitation those listed in any of International Patent Publications WO/2007/137187 (Int'l Appl. No. PCT/US2007/069286), published Nov. 29, 2007; WO/2010/045318 (Int'l Appl. No. PCT/US2009/060630), published Apr. 22, 2010; WO/2010/093465 (Int'l Appl. No. PCT/US2010/000407), published Aug. 19, 2010; WO/2012/170715 (Int'l Appl. No. PCT/US2012/041393), published Dec. 13, 2012; WO/2014/089241 (Int'l Appl. No. PCT/US2013/073184), published Jun. 12, 2014; WO/2011/056688 (Int'l Appl. No. PCT/US2010/054366), published May 12, 2011; WO/2012/092336 (Int'l Appl. No. PCT/US2011/067527), published Jul. 5, 2012; WO/2015/116868 (Int'l Appl. No. PCT/US2015/013618), published Aug. 6, 2015; WO/2017/053915 (Int'l Appl. No. PCT/US2016/053614), published Mar. 30, 2017; and WO/2016/141169 (Int'l Appl. No. PCT/US2016/020657), published Sep. 9, 2016; each of which publications is incorporated by reference herein in its entirety.

The rules for the mappings can contain a variety of supplemental information. In some embodiments, the database contains prioritization criteria. For example, a treatment with more projected efficacy in a given setting can be preferred over a treatment projected to have lesser efficacy. A mapping derived from a certain setting, e.g., a clinical trial, may be prioritized over a mapping derived from another setting, e.g., cell culture experiments. A treatment with strong literature support may be prioritized over a treatment supported by more preliminary results. A treatment generally applied to the type of disease in question, e.g., cancer of a certain tissue origin, may be prioritized over a treatment that is not indicated for that particular disease. Mappings can include both positive and negative correlations between a treatment and a molecular profiling result. In a non-limiting example, one mapping might suggest use of a kinase inhibitor like erlotinib against a tumor having an activating mutation in EGFR, whereas another mapping might suggest against that treatment if the EGFR also has a drug resistance mutation. Similarly, a treatment might be indicated as effective in cells that overexpress a certain gene or protein but indicated as not effective if the gene or protein is underexpressed.

The selection of a candidate treatment for an individual can be based on molecular profiling results from any one or more of the methods described. In embodiments, selection of a candidate treatment for an individual is based on molecular profiling results from more than one of the methods described. For example, selection of treatment for an individual can be based on molecular profiling results from ISH alone, IHC alone, or NGS analysis alone. Alternately, selection can be based on results from multiple techniques, which results may be ranked according to a desired scheme, such by level of evidence. In some embodiments, sequencing reveals a drug resistance mutation so that the effected drug is not selected even if techniques such as IHC indicate differential expression of the target molecule. Any such contraindication, e.g., differential expression or mutation of another gene or gene product may override selection of a treatment.

An illustrative listing of microarray expression results versus predicted treatments is presented in Table 2. As disclosed herein, molecular profiling is performed to determine whether a gene or gene product is differentially expressed in a sample as compared to a control. The expression status of the gene or gene product is used to select agents that are predicted to be efficacious or not. For example, Table 2 shows that overexpression of the ADA gene or protein points to pentostatin as a possible treatment. On the other hand, underexpression of the ADA gene or protein implicates resistance to cytarabine, suggesting that cytarabine is not an optimal treatment.

TABLE 2
Molecular Profiling Results and Predicted Treatments

| Gene Name | Expression Status | Candidate Agent(s) | Possible Resistance |
|---|---|---|---|
| ADA | Overexpressed | pentostatin | |
| ADA | Underexpressed | | cytarabine |
| AR | Overexpressed | abarelix, bicalutamide, | |
| | | flutamide, gonadorelin, | |
| | | goserelin, leuprolide | |
| ASNS | Underexpressed | asparaginase, | |
| | | pegaspargase | |
| BCRP (ABCG2) | Overexpressed | | cisplatin, carboplatin, |
| | | | irinotecan, topotecan |
| BRCA1 | Underexpressed | mitomycin | |
| BRCA2 | Underexpressed | mitomycin | |
| CD52 | Overexpressed | alemtuzumab | |
| CDA | Overexpressed | | cytarabine |
| CES2 | Overexpressed | irinotecan | |
| c-kit | Overexpressed | sorafenib, sunitinib, | |
| | | imatinib | |
| COX-2 | Overexpressed | celecoxib | |
| DCK | Overexpressed | gemcitabine | cytarabine |
| DHFR | Underexpressed | methotrexate, | |
| | | pemetrexed | |
| DHFR | Overexpressed | | methotrexate |
| DNMT1 | Overexpressed | azacitidine, decitabine | |
| DNMT3A | Overexpressed | azacitidine, decitabine | |
| DNMT3B | Overexpressed | azacitidine, decitabine | |
| EGFR | Overexpressed | erlotinib, gefitinib, | |
| | | cetuximab, panitumumab | |
| EML4-ALK | Overexpressed (present) | crizotinib | |
| EPHA2 | Overexpressed | dasatinib | |
| ER | Overexpressed | anastrazole, exemestane, | |
| | | fulvestrant, letrozole, | |
| | | megestrol, tamoxifen, | |
| | | medroxyprogesterone, | |
| | | toremifene, | |
| | | aminoglutethimide | |
| ERCC1 | Overexpressed | | carboplatin, cisplatin |
| GART | Underexpressed | pemetrexed | |
| HER-2 (ERBB2) | Overexpressed | trastuzumab, lapatinib | |
| HIF-1α | Overexpressed | sorafenib, sunitinib, | |
| | | bevacizumab | |
| IκB-α | Overexpressed | bortezomib | |
| MGMT | Underexpressed | temozolomide | |
| MGMT | Overexpressed | | temozolomide |
| MRP1 (ABCC1) | Overexpressed | | etoposide, paclitaxel, |
| | | | docetaxel, |
| | | | vinblastine, |
| | | | vinorelbine, |
| | | | topotecan, teniposide |
| P-gp (ABCB1) | Overexpressed | | doxorubicin, |
| | | | etoposide, epirubicin, |
| | | | paclitaxel, docetaxel, |
| | | | vinblastine, |
| | | | vinorelbine, |
| | | | topotecan, teniposide, |
| | | | liposomal |
| | | | doxorubicin |
| PDGFR-α | Overexpressed | sorafenib, sunitinib, | |
| | | imatinib | |
| PDGFR-β | Overexpressed | sorafenib, sunitinib, | |
| | | imatinib | |
| PR | Overexpressed | exemestane, fulvestrant, | |
| | | gonadorelin, goserelin, | |
| | | medroxyprogesterone, | |
| | | megestrol, tamoxifen, | |
| | | toremifene | |
| RARA | Overexpressed | ATRA | |
| RRM1 | Underexpressed | gemcitabine, | |
| | | hydroxyurea | |
| RRM2 | Underexpressed | gemcitabine, | |
| | | hydroxyurea | |
| RRM2B | Underexpressed | gemcitabine, | |
| | | hydroxyurea | |
| RXR-α | Overexpressed | bexarotene | |
| RXR-β | Overexpressed | bexarotene | |
| SPARC | Overexpressed | nab-paclitaxel | |
| SRC | Overexpressed | dasatinib | |
| SSTR2 | Overexpressed | octreotide | |
| SSTR5 | Overexpressed | octreotide | |
| TOPO I | Overexpressed | irinotecan, topotecan | |
| TOPO IIα | Overexpressed | doxorubicin, epirubicin, | |
| | | liposomal- doxorubicin | |
| TOPO IIβ | Overexpressed | doxorubicin, epirubicin, | |
| | | liposomal- doxorubicin | |
| TS | Underexpressed | capecitabine, 5- | |
| | | fluorouracil, pemetrexed | |
| TS | Overexpressed | | capecitabine, 5- |
| | | | fluorouracil |
| VDR | Overexpressed | calcitriol, cholecalciferol | |
| VEGFR1 (Flt1) | Overexpressed | sorafenib, sunitinib, | |
| | | bevacizumab | |
| VEGFR2 | Overexpressed | sorafenib, sunitinib, | |
| | | bevacizumab | |
| VHL | Underexpressed | sorafenib, sunitinib | |

Further drug associations and rules that can be used in embodiments of the invention are found in any of International Patent Publications WO/2007/137187 (Int'l Appl. No. PCT/US2007/069286), published Nov. 29, 2007; WO/2010/045318 (Int'l Appl. No. PCT/US2009/060630), published Apr. 22, 2010; WO/2010/093465 (Int'l Appl. No. PCT/US2010/000407), published Aug. 19, 2010; WO/2012/170715 (Int'l Appl. No. PCT/US2012/041393), published Dec. 13, 2012; WO/2014/089241 (Int'l Appl. No. PCT/US2013/073184), published Jun. 12, 2014; WO/2011/056688 (Int'l Appl. No. PCT/US2010/054366), published May 12, 2011; WO/2012/092336 (Int'l Appl. No. PCT/US2011/067527), published Jul. 5, 2012; WO/2015/116868 (Int'l Appl. No. PCT/US2015/013618), published Aug. 6, 2015; WO/2017/053915 (Int'l Appl. No. PCT/US2016/053614), published Mar. 30, 2017; and WO/2016/141169 (Int'l Appl. No. PCT/US2016/020657), published Sep. 9, 2016; each of which publications is incorporated by reference herein in its entirety. See e.g., “Table 4: Rules Summary for Treatment Selection” of WO/2011/056688.

The efficacy of various therapeutic agents given particular assay results, can be derived from reviewing, analyzing and rendering conclusions on empirical evidence, such as that is available the medical literature or other medical knowledge base. The results are used to guide the selection of certain therapeutic agents in a prioritized list for use in treatment of an individual. When molecular profiling results are obtained, e.g., differential expression or mutation of a gene or gene product, the results can be compared against the database to guide treatment selection. The set of rules in the database can be updated as new treatments and new treatment data become available. In some embodiments, the rules database is updated continuously. In some embodiments, the rules database is updated on a periodic basis. Any relevant correlative or comparative approach can be used to compare the molecular profiling results to the rules database. In one embodiment, a gene or gene product is identified as differentially expressed by molecular profiling. The rules database is queried to select entries for that gene or gene product. Treatment selection information selected from the rules database is extracted and used to select a treatment. The information, e.g., to recommend or not recommend a particular treatment, can be dependent on whether the gene or gene product is over or underexpressed, or has other abnormalities at the genetic or protein levels as compared to a reference. In some cases, multiple rules and treatments may be pulled from a database comprising the comprehensive rules set depending on the results of the molecular profiling. In some embodiments, the treatment options are presented in a prioritized list. In some embodiments, the treatment options are presented without prioritization information. In either case, an individual, e.g., the treating physician or similar caregiver may choose from the available options.

The methods described herein are used to prolong survival of a subject by providing personalized treatment. In some embodiments, the subject has been previously treated with one or more therapeutic agents to treat the disease, e.g., a cancer. The cancer may be refractory to one of these agents, e.g., by acquiring drug resistance mutations. In some embodiments, the cancer is metastatic. In some embodiments, the subject has not previously been treated with one or more therapeutic agents identified by the method. Using molecular profiling, candidate treatments can be selected regardless of the stage, anatomical location, or anatomical origin of the cancer cells.

Progression-free survival (PFS) denotes the chances of staying free of disease progression for an individual or a group of individuals suffering from a disease, e.g., a cancer, after initiating a course of treatment. It can refer to the percentage of individuals in a group whose disease is likely to remain stable (e.g., not show signs of progression) after a specified duration of time. Progression-free survival rates are an indication of the effectiveness of a particular treatment. Similarly, disease-free survival (DFS) denotes the chances of staying free of disease after initiating a particular treatment for an individual or a group of individuals suffering from a cancer. It can refer to the percentage of individuals in a group who are likely to be free of disease after a specified duration of time. Disease-free survival rates are an indication of the effectiveness of a particular treatment. Treatment strategies can be compared on the basis of the PFS or DFS that is achieved in similar groups of patients. Disease-free survival is often used with the term overall survival when cancer survival is described.

The candidate treatment selected by molecular profiling according to the invention can be compared to a non-molecular profiling selected treatment by comparing the progression free survival (PFS) using therapy selected by molecular profiling (period B) with PFS for the most recent therapy on which the patient has just progressed (period A). In one setting, a PFS(B)/PFS(A) ratio≥1.3 was used to indicate that the molecular profiling selected therapy provides benefit for patient (Robert Temple, Clinical measurement in drug evaluation. Edited by Wu Ningano and G. T. Thicker John Wiley and Sons Ltd. 1995; Von Hoff D. D. Clin Can Res. 4: 1079, 1999: Dhani et al. Clin Cancer Res. 15: 118-123, 2009). Other methods of comparing the treatment selected by molecular profiling to a non-molecular profiling selected treatment include determining response rate (RECIST) and percent of patients without progression or death at 4 months. The term “about” as used in the context of a numerical value for PFS means a variation of +/−ten percent (10%) relative to the numerical value. The PFS from a treatment selected by molecular profiling can be extended by at least 10%, 15%, 20%, 30%, 40%, 50%, 60%, 70%, 80%, or at least 90% as compared to a non-molecular profiling selected treatment. In some embodiments, the PFS from a treatment selected by molecular profiling can be extended by at least 100%, 150%, 200%, 300%, 400%, 500%, 600%, 700%, 800%, 900%, or at least about 1000% as compared to a non-molecular profiling selected treatment. In yet other embodiments, the PFS ratio (PFS on molecular profiling selected therapy or new treatment/PFS on prior therapy or treatment) is at least about 1.3. In yet other embodiments, the PFS ratio is at least about 1.1, 1.2, 1.3, 1.4, 1.5, 1.6, 1.7, 1.8, 1.9, or 2.0. In yet other embodiments, the PFS ratio is at least about 3, 4, 5, 6, 7, 8, 9 or 10.

Similarly, the DFS can be compared in patients whose treatment is selected with or without molecular profiling. In embodiments, DFS from a treatment selected by molecular profiling is extended by at least 10%, 15%, 20%, 30%, 40%, 50%, 60%, 70%, 80%, or at least 90% as compared to a non-molecular profiling selected treatment. In some embodiments, the DFS from a treatment selected by molecular profiling can be extended by at least 100%, 150%, 200%, 300%, 400%, 500%, 600%, 700%, 800%, 900%, or at least about 1000% as compared to a non-molecular profiling selected treatment. In yet other embodiments, the DFS ratio (DFS on molecular profiling selected therapy or new treatment/DFS on prior therapy or treatment) is at least about 1.3. In yet other embodiments, the DFS ratio is at least about 1.1, 1.2, 1.3, 1.4, 1.5, 1.6, 1.7, 1.8, 1.9, or 2.0. In yet other embodiments, the DFS ratio is at least about 3, 4, 5, 6, 7, 8, 9 or 10.

In some embodiments, the candidate treatment of the invention will not increase the PFS ratio or the DFS ratio in the patient, nevertheless molecular profiling provides invaluable patient benefit. For example, in some instances no preferable treatment has been identified for the patient. In such cases, molecular profiling provides a method to identify a candidate treatment where none is currently identified. The molecular profiling may extend PFS, DFS or lifespan by at least 1 week, 2 weeks, 3 weeks, 4 weeks, 1 month, 5 weeks, 6 weeks, 7 weeks, 8 weeks, 2 months, 9 weeks, 10 weeks, 11 weeks, 12 weeks, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months or 2 years. The molecular profiling may extend PFS, DFS or lifespan by at least 2 1/2% years, 3 years, 4 years, 5 years, or more. In some embodiments, the methods of the invention improve outcome so that patient is in remission.

The effectiveness of a treatment can be monitored by other measures. A complete response (CR) comprises a complete disappearance of the disease: no disease is evident on examination, scans or other tests. A partial response (PR) refers to some disease remaining in the body, but there has been a decrease in size or number of the lesions by 30% or more. Stable disease (SD) refers to a disease that has remained relatively unchanged in size and number of lesions. Generally, less than a 50% decrease or a slight increase in size would be described as stable disease. Progressive disease (PD) means that the disease has increased in size or number on treatment. In some embodiments, molecular profiling according to the invention results in a complete response or partial response. In some embodiments, the methods of the invention result in stable disease. In some embodiments, the invention is able to achieve stable disease where non-molecular profiling results in progressive disease.

### Computer Systems

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, for example Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2.sup.nd ed., 2001). See U.S. Pat. No. 6,420,108.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention relates to embodiments that include methods for providing genetic information over networks such as the Internet as shown in U.S. Ser. Nos. 10/197,621, 10/063,559 (U.S. Publication Number 20020183936), U.S. Ser. Nos. 10/065,856, 10/065,868, 10/328,818, 10/328,872, 10/423,403, and 60/482,389. For example, one or more molecular profiling techniques can be performed in one location, e.g., a city, state, country or continent, and the results can be transmitted to a different city, state, country or continent. Treatment selection can then be made in whole or in part in the second location. The methods of the invention comprise transmittal of information between different locations.

Conventional data networking, application development and other functional aspects of the systems (and components of the individual operating components of the systems) may not be described in detail herein but are part of the invention. Furthermore, the connecting lines shown in the various figures contained herein are intended to represent illustrative functional relationships and/or physical couplings between the various elements. It should be noted that many alternative or additional functional relationships or physical connections may be present in a practical system.

The various system components discussed herein may include one or more of the following: a host server or other computing systems including a processor for processing digital data; a memory coupled to the processor for storing digital data; an input digitizer coupled to the processor for inputting digital data; an application program stored in the memory and accessible by the processor for directing processing of digital data by the processor; a display device coupled to the processor and memory for displaying information derived from digital data processed by the processor; and a plurality of databases. Various databases used herein may include: patient data such as family history, demography and environmental data, biological sample data, prior treatment and protocol data, patient clinical data, molecular profiling data of biological samples, data on therapeutic drug agents and/or investigative drugs, a gene library, a disease library, a drug library, patient tracking data, file management data, financial management data, billing data and/or like data useful in the operation of the system. As those skilled in the art will appreciate, user computer may include an operating system (e.g., Windows NT, 95/98/2000, OS2, UNIX, Linux, Solaris, MacOS, etc.) as well as various conventional support software and drivers typically associated with computers. The computer may include any suitable personal computer, network computer, workstation, minicomputer, mainframe or the like. User computer can be in a home or medical/business environment with access to a network. In an illustrative embodiment, access is through a network or the Internet through a commercially-available web-browser software package.

As used herein, the term “network” shall include any electronic communications means which incorporates both hardware and software components of such. Communication among the parties may be accomplished through any suitable communication channels, such as, for example, a telephone network, an extranet, an intranet, Internet, point of interaction device, personal digital assistant (e.g., Palm Pilot®, Blackberry®), cellular phone, kiosk, etc.), online communications, satellite communications, off-line communications, wireless communications, transponder communications, local area network (LAN), wide area network (WAN), networked or linked devices, keyboard, mouse and/or any suitable communication or data input modality. Moreover, although the system is frequently described herein as being implemented with TCP/IP communications protocols, the system may also be implemented using IPX, Appletalk, IP-6, NetBIOS, OSI or any number of existing or future protocols. If the network is in the nature of a public network, such as the Internet, it may be advantageous to presume the network to be insecure and open to eavesdroppers. Specific information related to the protocols, standards, and application software used in connection with the Internet is generally known to those skilled in the art and, as such, need not be detailed herein. See, for example, DILIP NAIK, INTERNET STANDARDS AND PROTOCOLS (1998); JAVA 2 COMPLETE, various authors, (Sybex 1999); DEBORAH RAY AND ERIC RAY, MASTERING HTML 4.0 (1997); and LOSHIN, TCP/IP CLEARLY EXPLAINED (1997) and DAVID GOURLEY AND BRIAN TOTTY, HTTP, THE DEFINITIVE GUIDE (2002), the contents of which are hereby incorporated by reference.

The various system components may be independently, separately or collectively suitably coupled to the network via data links which includes, for example, a connection to an Internet Service Provider (ISP) over the local loop as is typically used in connection with standard modem communication, cable modem, Dish networks, ISDN, Digital Subscriber Line (DSL), or various wireless communication methods, see, e.g., GILBERT HELD, UNDERSTANDING DATA COMMUNICATIONS (1996), which is hereby incorporated by reference. It is noted that the network may be implemented as other types of networks, such as an interactive television (ITV) network. Moreover, the system contemplates the use, sale or distribution of any goods, services or information over any network having similar functionality described herein.

As used herein, “transmit” may include sending electronic data from one system component to another over a network connection. Additionally, as used herein, “data” may include encompassing information such as commands, queries, files, data for storage, and the like in digital or any other form.

The system contemplates uses in association with web services, utility computing, pervasive and individualized computing, security and identity solutions, autonomic computing, commodity computing, mobility and wireless solutions, open source, biometrics, grid computing and/or mesh computing.

Any databases discussed herein may include relational, hierarchical, graphical, or object-oriented structure and/or any other database configurations. Common database products that may be used to implement the databases include DB2 by IBM (White Plains, NY), various database products available from Oracle Corporation (Redwood Shores, CA), Microsoft Access or Microsoft SQL Server by Microsoft Corporation (Redmond, Washington), or any other suitable database product. Moreover, the databases may be organized in any suitable manner, for example, as data tables or lookup tables. Each record may be a single file, a series of files, a linked series of data fields or any other data structure. Association of certain data may be accomplished through any desired data association technique such as those known or practiced in the art. For example, the association may be accomplished either manually or automatically. Automatic association techniques may include, for example, a database search, a database merge, GREP, AGREP, SQL, using a key field in the tables to speed searches, sequential searches through all the tables and files, sorting records in the file according to a known order to simplify lookup, and/or the like. The association step may be accomplished by a database merge function, for example, using a “key field” in pre-selected databases or data sectors.

More particularly, a “key field” partitions the database according to the high-level class of objects defined by the key field. For example, certain types of data may be designated as a key field in a plurality of related data tables and the data tables may then be linked on the basis of the type of data in the key field. The data corresponding to the key field in each of the linked data tables is preferably the same or of the same type. However, data tables having similar, though not identical, data in the key fields may also be linked by using AGREP, for example. In accordance with one embodiment, any suitable data storage technique may be used to store data without a standard format. Data sets may be stored using any suitable technique, including, for example, storing individual files using an ISO/IEC 7816-4 file structure; implementing a domain whereby a dedicated file is selected that exposes one or more elementary files containing one or more data sets; using data sets stored in individual files using a hierarchical filing system; data sets stored as records in a single file (including compression, SQL accessible, hashed vione or more keys, numeric, alphabetical by first tuple, etc.); Binary Large Object (BLOB); stored as ungrouped data elements encoded using ISO/IEC 7816-6 data elements; stored as ungrouped data elements encoded using ISO/IEC Abstract Syntax Notation (ASN.1) as in ISO/IEC 8824 and 8825; and/or other proprietary techniques that may include fractal compression methods, image compression methods, etc.

In one illustrative embodiment, the ability to store a wide variety of information in different formats is facilitated by storing the information as a BLOB. Thus, any binary information can be stored in a storage space associated with a data set. The BLOB method may store data sets as ungrouped data elements formatted as a block of binary via a fixed memory offset using either fixed storage allocation, circular queue techniques, or best practices with respect to memory management (e.g., paged memory, least recently used, etc.). By using BLOB methods, the ability to store various data sets that have different formats facilitates the storage of data by multiple and unrelated owners of the data sets. For example, a first data set which may be stored may be provided by a first party, a second data set which may be stored may be provided by an unrelated second party, and yet a third data set which may be stored, may be provided by a third party unrelated to the first and second party. Each of these three illustrative data sets may contain different information that is stored using different data storage formats and/or techniques. Further, each data set may contain subsets of data that also may be distinct from other subsets.

As stated above, in various embodiments, the data can be stored without regard to a common format. However, in one illustrative embodiment, the data set (e.g., BLOB) may be annotated in a standard manner when provided for manipulating the data. The annotation may comprise a short header, trailer, or other appropriate indicator related to each data set that is configured to convey information useful in managing the various data sets. For example, the annotation may be called a “condition header”, “header”, “trailer”, or “status”, herein, and may comprise an indication of the status of the data set or may include an identifier correlated to a specific issuer or owner of the data. Subsequent bytes of data may be used to indicate for example, the identity of the issuer or owner of the data, user, transaction/membership account identifier or the like. Each of these condition annotations are further discussed herein.

The data set annotation may also be used for other types of status information as well as various other purposes. For example, the data set annotation may include security information establishing access levels. The access levels may, for example, be configured to permit only certain individuals, levels of employees, companies, or other entities to access data sets, or to permit access to specific data sets based on the transaction, issuer or owner of data, user or the like. Furthermore, the security information may restrict/permit only certain actions such as accessing, modifying, and/or deleting data sets. In one example, the data set annotation indicates that only the data set owner or the user are permitted to delete a data set, various identified users may be permitted to access the data set for reading, and others are altogether excluded from accessing the data set. However, other access restriction parameters may also be used allowing various entities to access a data set with various permission levels as appropriate. The data, including the header or trailer may be received by a standalone interaction device configured to add, delete, modify, or augment the data in accordance with the header or trailer.

One skilled in the art will also appreciate that, for security reasons, any databases, systems, devices, servers or other components of the system may consist of any combination thereof at a single location or at multiple locations, wherein each database or system includes any of various suitable security features, such as firewalls, access codes, encryption, decryption, compression, decompression, and/or the like.

The computing unit of the web client may be further equipped with an Internet browser connected to the Internet or an intranet using standard dial-up, cable, DSL or any other Internet protocol known in the art. Transactions originating at a web client may pass through a firewall in order to prevent unauthorized access from users of other networks. Further, additional firewalls may be deployed between the varying components of CMS to further enhance security.

Firewall may include any hardware and/or software suitably configured to protect CMS components and/or enterprise computing resources from users of other networks. Further, a firewall may be configured to limit or restrict access to various systems and components behind the firewall for web clients connecting through a web server. Firewall may reside in varying configurations including Stateful Inspection, Proxy based and Packet Filtering among others. Firewall may be integrated within an web server or any other CMS components or may further reside as a separate entity.

The computers discussed herein may provide a suitable website or other Internet-based graphical user interface which is accessible by users. In one embodiment, the Microsoft Internet Information Server (IIS), Microsoft Transaction Server (MTS), and Microsoft SQL Server, are used in conjunction with the Microsoft operating system, Microsoft NT web server software, a Microsoft SQL Server database system, and a Microsoft Commerce Server. Additionally, components such as Access or Microsoft SQL Server, Oracle, Sybase, Informix MySQL, Interbase, etc., may be used to provide an Active Data Object (ADO) compliant database management system.

Any of the comnuinications, inputs, storage, databases or displays discussed herein may be facilitated through a website having web pages. The term “web page” as it is used herein is not meant to limit the type of documents and applications that might be used to interact with the user. For example, a typical website Emghl include. In addition to standard. HTML documnents, various forms-Java applets, JavaScript, active server pages (ASP), common gateway interface scripts (CGi), extensible markup language (XML), dynamic HTML, cascading style sheets (CSS), helper applications, plug-ins, and the like. A server may include a web service that receives a request from a web server, the request including a UPI (e.g., http://yahoo.com/stockquotes/ge) and an IP address (e.g. 123.56.789.234). The web server retrieves the appropriate web pages and sends the data or applications for the web pages to the IP address, Web services are applications that are capable of interacting with other applications over a communications means, such as the internet. Web services are typically based on standards or protocols such as XML, XSLT, SOAP, WSDL and UDDI. Web services methods are well known in the art, and are covered in many standard texts. See, e.g., ALEX NGHIEM. IT WEB SERVICES: A ROADMAP FOR THE ENTERPRISE (2003), hereby incorporated by reference.

The web-based clinical database for the system and method of the present invention preferably has the ability to upload and store clinical data files in native formats and is searchable on any clinical parameter. The database is also scalable and may use an EAV data model (metadata) to enter clinical annotations from any study for easy integration with other studies. In addition, the web-based clinical database is flexible and may be XML and XSLT enabled to be able to add user customized questions dynamically. Further, the database includes exportability to CDISC ODM.

Practitioners will also appreciate that there are a number of methods for displaying data within a browser-based document. Data may be represented as standard text or within a fixed list, scrollable list, drop-down list, editable text field, fixed text field, pop-up window, and the like. Likewise, there are a number of methods available for modifying data in a web page such as, for example, free text entry using a keyboard, selection of menu items, check boxes, option boxes, and the like.

The system and method may be described herein in terms of functional block components, screen shots, optional selections and various processing steps. It should be appreciated that such functional blocks may be realized by any number of hardware and/or software components configured to perform the specified functions. For example, the system may employ various integrated circuit components, e.g., memory elements, processing elements, logic elements, look-up tables, and the like, which may carry out a variety of functions under the control of one or more microprocessors or other control devices. Similarly, the software elements of the system may be implemented with any programming or scripting language such as C, C++, Macromedia Cold Fusion, Microsoft Active Server Pages, Java, COBOL, assembler, PERL, Visual Basic, SQL Stored Procedures, extensible markup language (XML), with the various algorithms being implemented with any combination of data structures, objects, processes, routines or other programming elements. Further, it should be noted that the system may employ any number of conventional techniques for data transmission, signaling, data processing, network control, and the like. Still further, the system could be used to detect or prevent security issues with a client-side scripting language, such as JavaScript, VBScript or the like. For a basic introduction of cryptography and network security, see any of the following references: (1) “Applied Cryptography: Protocols, Algorithms, And Source Code In C,” by Bruce Schneier, published by John Wiley & Sons (second edition, 1995); (2) “Java Cryptography” by Jonathan Knudson, published by O'Reilly & Associates (1998); (3) “Cryptography & Network Security: Principles & Practice” by William Stallings, published by Prentice Hall; all of which are hereby incorporated by reference.

As used herein, the term “end user”, “consumer”, “customer”, “client”, “treating physician”, “hospital”, or “business” may be used interchangeably with each other, and each shall mean any person, entity, machine, hardware, software or business. Each participant is equipped with a computing device in order to interact with the system and facilitate online data access and data input. The customer has a computing unit in the form of a personal computer, although other types of computing units may be used including laptops, notebooks, hand held computers, set-top boxes, cellular telephones, touch-tone telephones and the like. The owner/operator of the system and method of the present invention has a computing unit implemented in the form of a computer-server, although other implementations are contemplated by the system including a computing center shown as a main frame computer, a mini-computer, a PC server, a network of computers located in the same of different geographic locations, or the like. Moreover, the system contemplates the use, sale or distribution of any goods, services or information over any network having similar functionality described herein.

In one illustrative embodiment, each client customer may be issued an “account” or “account number”. As used herein, the account or account number may include any device, code, number, letter, symbol, digital certificate, smart chip, digital signal, analog signal, biometric or other identifier/indicia suitably configured to allow the consumer to access, interact with or communicate with the system (e.g., one or more of an authorization/access code, personal identification number (PIN), Internet code, other identification code, and/or the like). The account number may optionally be located on or associated with a charge card, credit card, debit card, prepaid card, embossed card, smart card, magnetic stripe card, bar code card, transponder, radio frequency card or an associated account. The system may include or interface with any of the foregoing cards or devices, or a fob having a transponder and RFID reader in RF communication with the fob. Although the system may include a fob embodiment, the invention is not to be so limited. Indeed, system may include any device having a transponder which is configured to communicate with RFID reader via RF communication. Typical devices may include, for example, a key ring, tag, card, cell phone, wristwatch or any such form capable of being presented for interrogation. Moreover, the system, computing unit or device discussed herein may include a “pervasive computing device,” which may include a traditionally non-computerized device that is embedded with a computing unit. The account number may be distributed and stored in any form of plastic, electronic, magnetic, radio frequency, wireless, audio and/or optical device capable of transmitting or downloading data from itself to a second device.

As will be appreciated by one of ordinary skill in the art, the system may be embodied as a customization of an existing system, an add-on product, upgraded software, a standalone system, a distributed system, a method, a data processing system, a device for data processing, and/or a computer program product. Accordingly, the system may take the form of an entirely software embodiment, an entirely hardware embodiment, or an embodiment combining aspects of both software and hardware. Furthermore, the system may take the form of a computer program product on a computer-readable storage medium having computer-readable program code means embodied in the storage medium. Any suitable computer-readable storage medium may be used, including hard disks, CD-ROM, optical storage devices, magnetic storage devices, and/or the like.

The system and method is described herein with reference to screen shots, block diagrams and flowchart illustrations of methods, apparatus (e.g., systems), and computer program products according to various embodiments. It will be understood that each functional block of the block diagrams and the flowchart illustrations, and combinations of functional blocks in the block diagrams and flowchart illustrations, respectively, can be implemented by computer program instructions.

These computer program instructions may be loaded onto a general purpose computer, special purpose computer, or other programmable data processing apparatus to produce a machine, such that the instructions that execute on the computer or other programmable data processing apparatus create means for implementing the functions specified in the flowchart block or blocks. These computer program instructions may also be stored in a computer-readable memory that can direct a computer or other programmable data processing apparatus to function in a particular manner, such that the instructions stored in the computer-readable memory produce an article of manufacture including instruction means which implement the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions which execute on the computer or other programmable apparatus provide steps for implementing the functions specified in the flowchart block or blocks.

Accordingly, functional blocks of the block diagrams and flowchart illustrations support combinations of means for performing the specified functions, combinations of steps for performing the specified functions, and program instruction means for performing the specified functions. It will also be understood that each functional block of the block diagrams and flowchart illustrations, and combinations of functional blocks in the block diagrams and flowchart illustrations, can be implemented by either special purpose hardware-based computer systems which perform the specified functions or steps, or suitable combinations of special purpose hardware and computer instructions. Further, illustrations of the process flows and the descriptions thereof may make reference to user windows, web pages, websites, web forms, prompts, etc. Practitioners will appreciate that the illustrated steps described herein may comprise in any number of configurations including the use of windows, web pages, web forms, popup windows, prompts and the like. It should be further appreciated that the multiple steps as illustrated and described may be combined into single web pages and/or windows but have been expanded for the sake of simplicity. In other cases, steps illustrated and described as single process steps may be separated into multiple web pages and/or windows but have been combined for simplicity. Molecular Profiling Methods

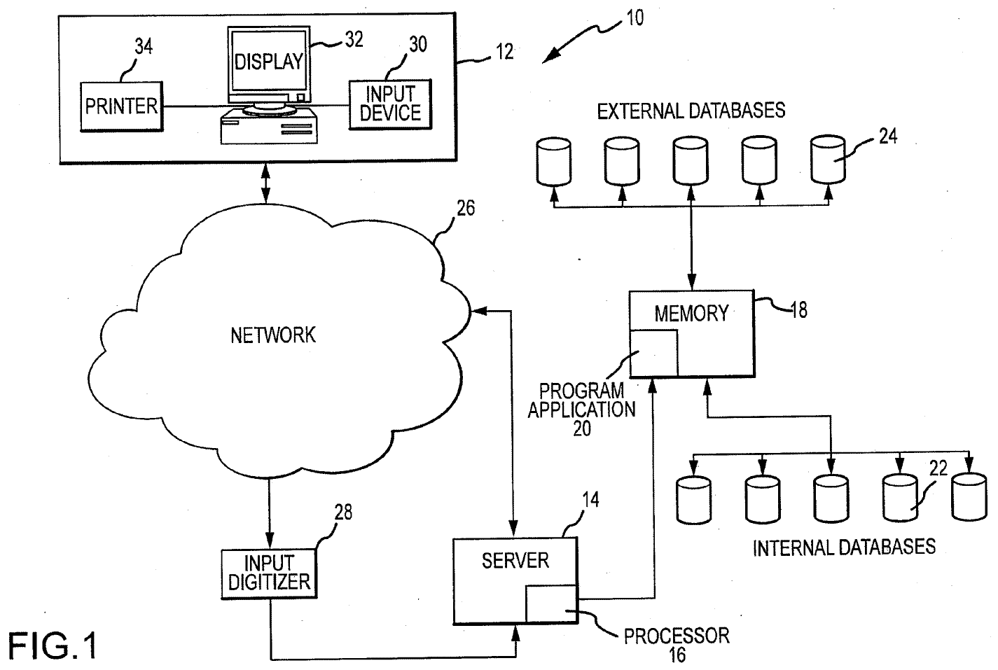

FIG. 1 illustrates a block diagram of an illustrative embodiment of a system 10 for determining individualized medical intervention for a particular disease state that uses molecular profiling of a patient's biological specimen. System 10 includes a user interface 12, a host server 14 including a processor 16 for processing data, a memory 18 coupled to the processor, an application program 20 stored in the memory 18 and accessible by the processor 16 for directing processing of the data by the processor 16, a plurality of internal databases 22 and external databases 24, and an interface with a wired or wireless communications network 26 (such as the Internet, for example). System 10 may also include an input digitizer 28 coupled to the processor 16 for inputting digital data from data that is received from user interface 12.

User interface 12 includes an input device 30 and a display 32 for inputting data into system 10 and for displaying information derived from the data processed by processor 16. User interface 12 may also include a printer 34 for printing the information derived from the data processed by the processor 16 such as patient reports that may include test results for targets and proposed drug therapies based on the test results.

Internal databases 22 may include, but are not limited to, patient biological sample/specimen information and tracking, clinical data, patient data, patient tracking, file management, study protocols, patient test results from molecular profiling, and billing information and tracking. External databases 24 nay include, but are not limited to, drug libraries, gene libraries, disease libraries, and public and private databases such as UniGene, OMIM, GO, TIGR, GenBank, KEGG and Biocarta.

Various methods may be used in accordance with system 10. FIG. 2 shows a flowchart of an illustrative embodiment of a method 50 for determining individualized medical intervention for a particular disease state that uses molecular profiling of a patient's biological specimen that is non disease specific. In order to determine a medical intervention for a particular disease state using molecular profiling that is independent of disease lineage diagnosis (i.e. not single disease restricted), at least one test is performed for at least one target from a biological sample of a diseased patient in step 52. A target is defined as any molecular finding that may be obtained from molecular testing. For example, a target may include one or more genes, one or more gene expressed proteins, one or more molecular mechanisms, and/or combinations of such. For example, the expression level of a target can be determined by the analysis of mRNA levels or the target or gene, or protein levels of the gene. Tests for finding such targets may include, but are not limited, fluorescent in-situ hybridization (FISH), in-situ hybridization (ISH), and other molecular tests known to those skilled in the art. PCR-based methods, such as real-time PCR or quantitative PCR can be used. Furthermore, microarray analysis, such as a comparative genomic hybridization (CGH) micro array, a single nucleotide polymorphism (SNP) microarray, a proteomic array, or antibody array analysis can also be used in the methods disclosed herein. In some embodiments, microarray analysis comprises identifying whether a gene is up-regulated or down-regulated relative to a reference with a significance of p<0.001. Tests or analyses of targets can also comprise immunohistochemical (IHC) analysis. In some embodiments, IHC analysis comprises determining whether 30% or more of a sample is stained, if the staining intensity is +2 or greater, or both.

Furthermore, the methods disclosed herein also including profiling more than one target. For example, the expression of a plurality of genes can be identified. Furthermore, identification of a plurality of targets in a sample can be by one method or by various means. For example, the expression of a first gene can be determined by one method and the expression level of a second gene determined by a different method. Alternatively, the same method can be used to detect the expression level of the first and second gene. For example, the first method can be IHC and the second by microarray analysis, such as detecting the gene expression of a gene.

In some embodiments, molecular profiling can also including identifying a genetic variant, such as a mutation, polymorphism (such as a SNP), deletion, or insertion of a target. For example, identifying a SNP in a gene can be determined by microarray analysis, real-time PCR, or sequencing. Other methods disclosed herein can also be used to identify variants of one or more targets.

Accordingly, one or more of the following may be performed: an IHC analysis in step 54, a microanalysis in step 56, and other molecular tests know to those skilled in the art in step 58.

Biological samples are obtained from diseased patients by taking a biopsy of a tumor, conducting minimally invasive surgery if no recent tumor is available, obtaining a sample of the patient's blood, or a sample of any other biological fluid including, but not limited to, cell extracts, nuclear extracts, cell lysates or biological products or substances of biological origin such as excretions, blood, sera, plasma, urine, sputum, tears, feces, saliva, membrane extracts, and the like.

In step 60, a determination is made as to whether one or more of the targets that were tested for in step 52 exhibit a change in expression compared to a normal reference for that particular target. In one illustrative method of the invention, an IHC analysis may be performed in step 54 and a determination as to whether any targets from the IHC analysis exhibit a change in expression is made in step 64 by determining whether 30% or more of the biological sample cells were+2 or greater staining for the particular target. It will be understood by those skilled in the art that there will be instances where+1 or greater staining will indicate a change in expression in that staining results may vary depending on the technician performing the test and type of target being tested. In another illustrative embodiment of the invention, a micro array analysis may be performed in step 56 and a determination as to whether any targets from the micro array analysis exhibit a change in expression is made in step 66 by identifying which targets are up-regulated or down-regulated by determining whether the fold change in expression for a particular target relative to a normal tissue of origin reference is significant at p<0.001. A change in expression may also be evidenced by an absence of one or more genes, gene expressed proteins, molecular mechanisms, or other molecular findings.

After determining which targets exhibit a change in expression in step 60, at least one non-disease specific agent is identified that interacts with each target having a changed expression in step 70. An agent may be any drug or compound having a therapeutic effect. A non-disease specific agent is a therapeutic drug or compound not previously associated with treating the patient's diagnosed disease that is capable of interacting with the target from the patient's biological sample that has exhibited a change in expression. Some of the non-disease specific agents that have been found to interact with specific targets found in different cancer patients are shown in Table 3 below.

TABLE 3
Illustrative target-drug associations

| Patients | Target(s) Found | Treatment(s) |
|---|---|---|
| Advanced Pancreatic Cancer | HER 2/neu | Trastuzumab |
| Advanced Pancreatic Cancer | EGFR, HIF 1α | Cetuximab, Sirolimus |
| Advanced Ovarian Cancer | ERCC3 | Irofulven |
| Advanced Adenoid Cystic | Vitamin D receptors, | Calcitriol, Flutamide |
| Carcinoma | Androgen receptors | |

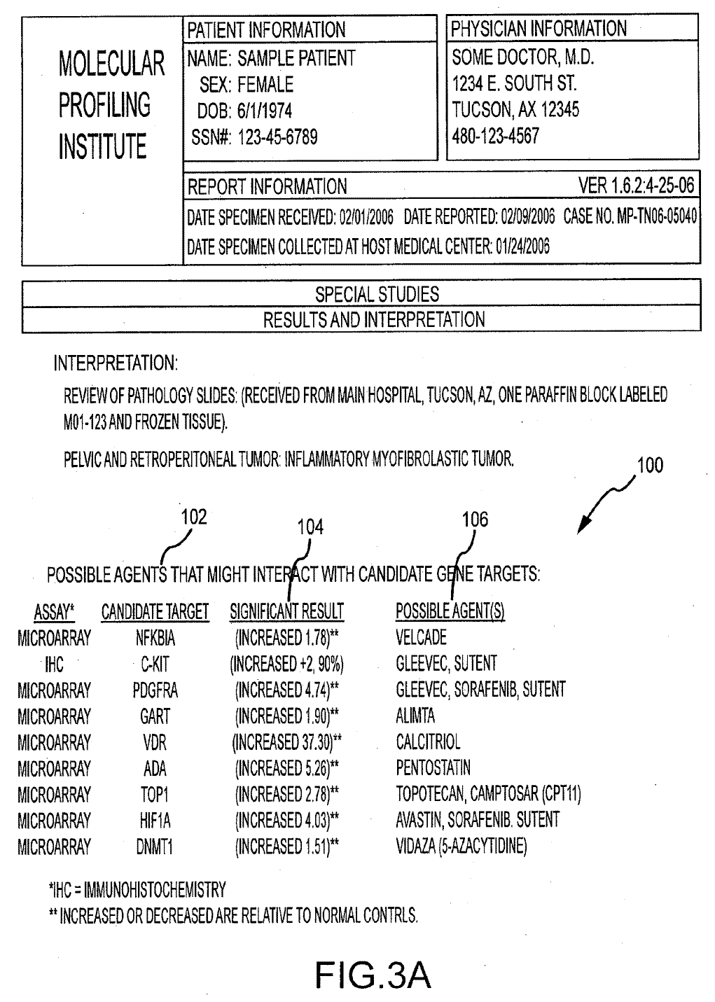

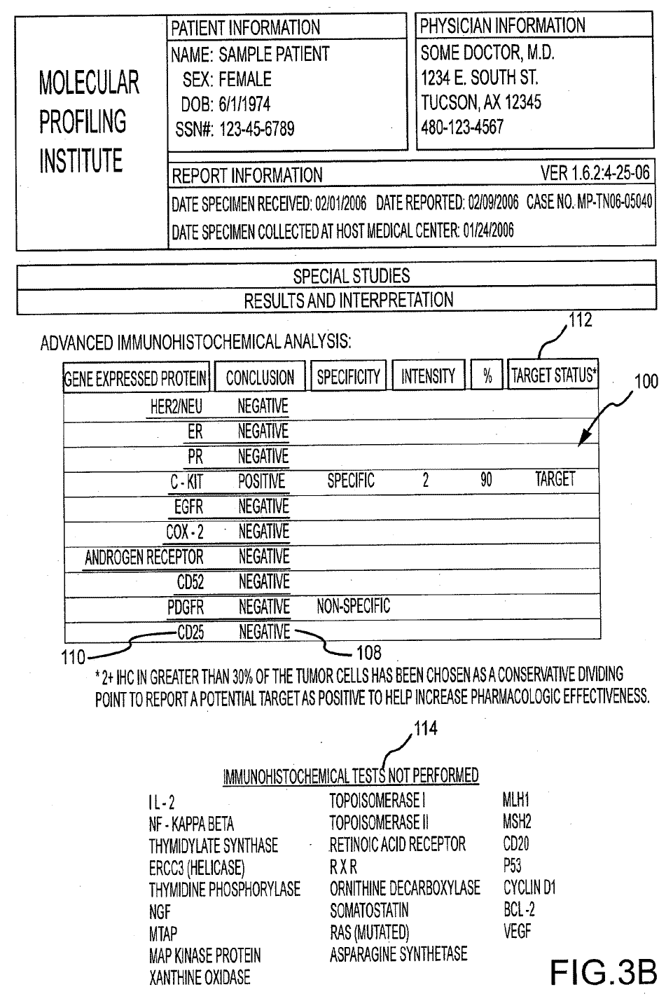

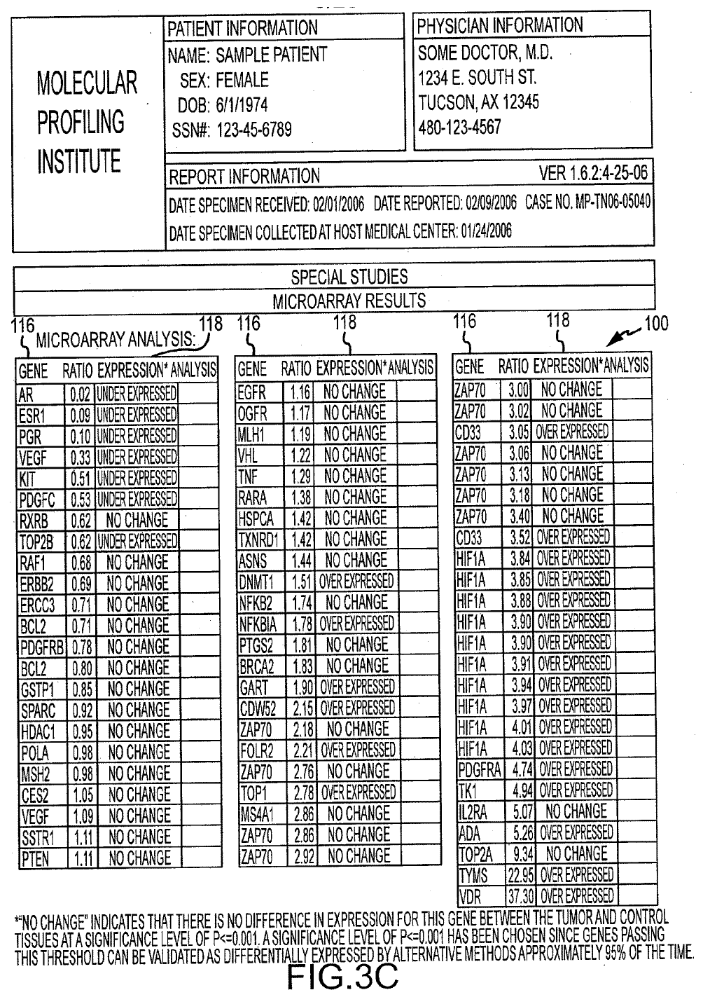

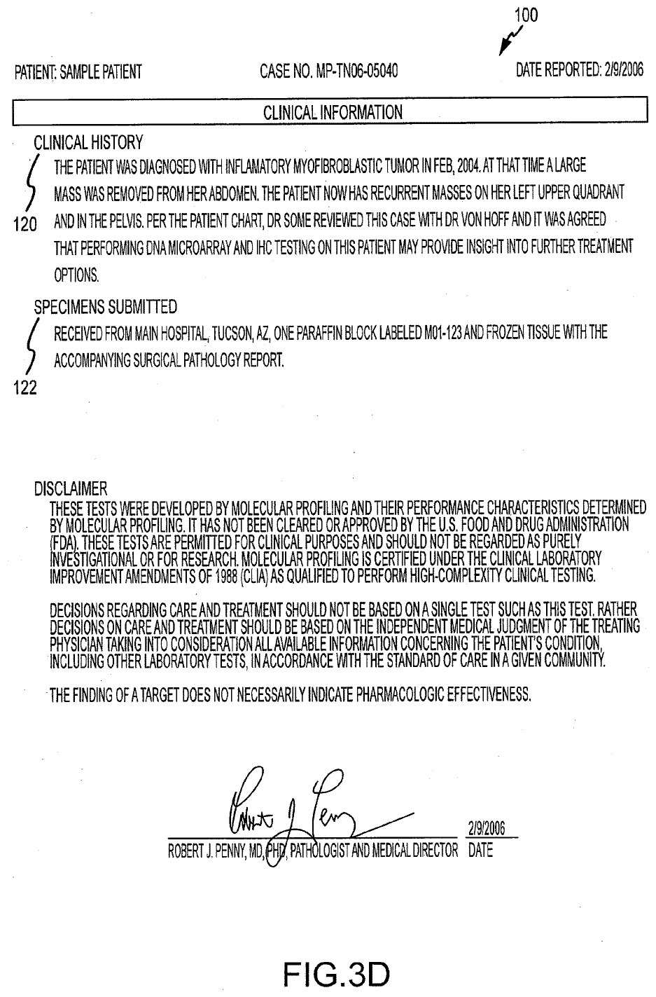

Finally, in step 80, a patient profile report may be provided which includes the patient's test results for various targets and any proposed therapies based on those results. An illustrative patient profile report 100 is shown in FIGS. 3A-3D. Patient profile report 100 shown in FIG. 3A identifies the targets tested 102, those targets tested that exhibited significant changes in expression 104, and proposed non-disease specific agents for interacting with the targets 106. Patient profile report 100 shown in FIG. 3B identifies the results 108 of immunohistochemical analysis for certain gene expressed proteins 110 and whether a gene expressed protein is a molecular target 112 by determining whether 30% or more of the tumor cells were+2 or greater staining. Report 100 also identifies immunohistochemical tests that were not performed 114. Patient profile report 100 shown in FIG. 3C identifies the genes analyzed 116 with a micro array analysis and whether the genes were under expressed or over expressed 118 compared to a reference. Finally, patient profile report 100 shown in FIG. 3D identifies the clinical history 120 of the patient and the specimens that were submitted 122 from the patient. Molecular profiling techniques can be performed anywhere, e.g., a foreign country, and the results sent by network to an appropriate party, e.g., the patient, a physician, lab or other party located remotely.

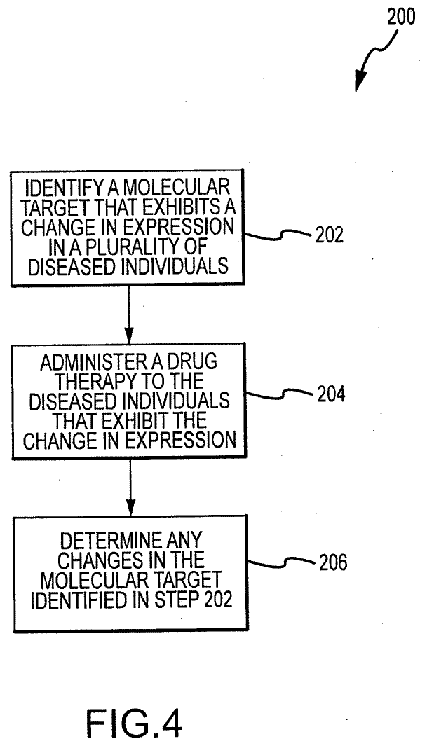

FIG. 4 shows a flowchart of an illustrative embodiment of a method 200 for identifying a drug therapy/agent capable of interacting with a target. In step 202, a molecular target is identified which exhibits a change in expression in a number of diseased individuals. Next, in step 204, a drug therapy/agent is administered to the diseased individuals. After drug therapy/agent administration, any changes in the molecular target identified in step 202 are identified in step 206 in order to determine if the drug therapy/agent administered in step 204 interacts with the molecular targets identified in step 202. If it is determined that the drug therapy/agent administered in step 204 interacts with a molecular target identified in step 202, the drug therapy/agent may be approved for treating patients exhibiting a change in expression of the identified molecular target instead of approving the drug therapy/agent for a particular disease.

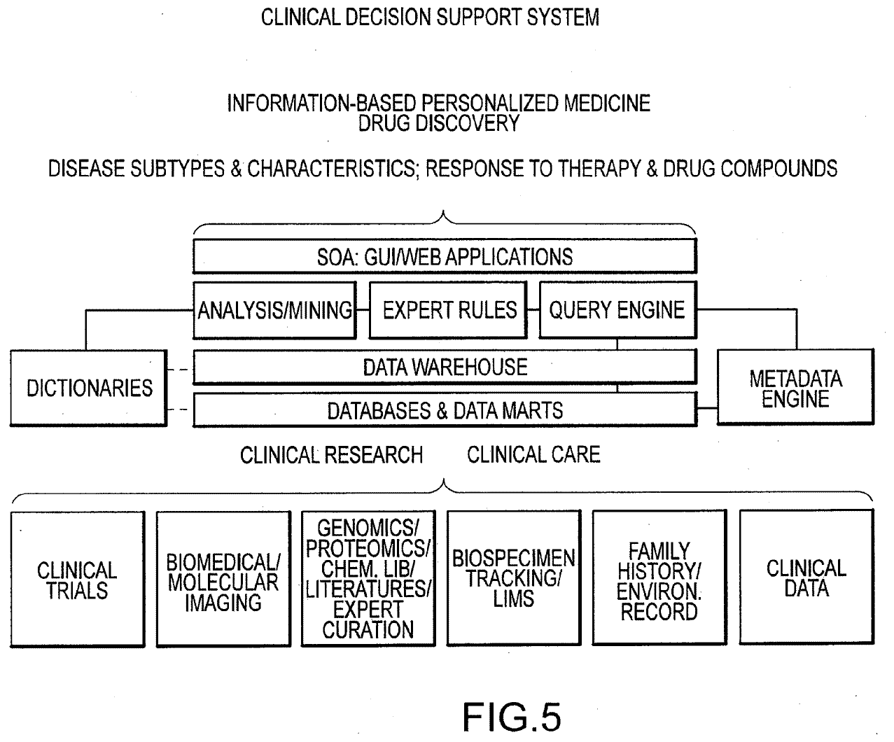

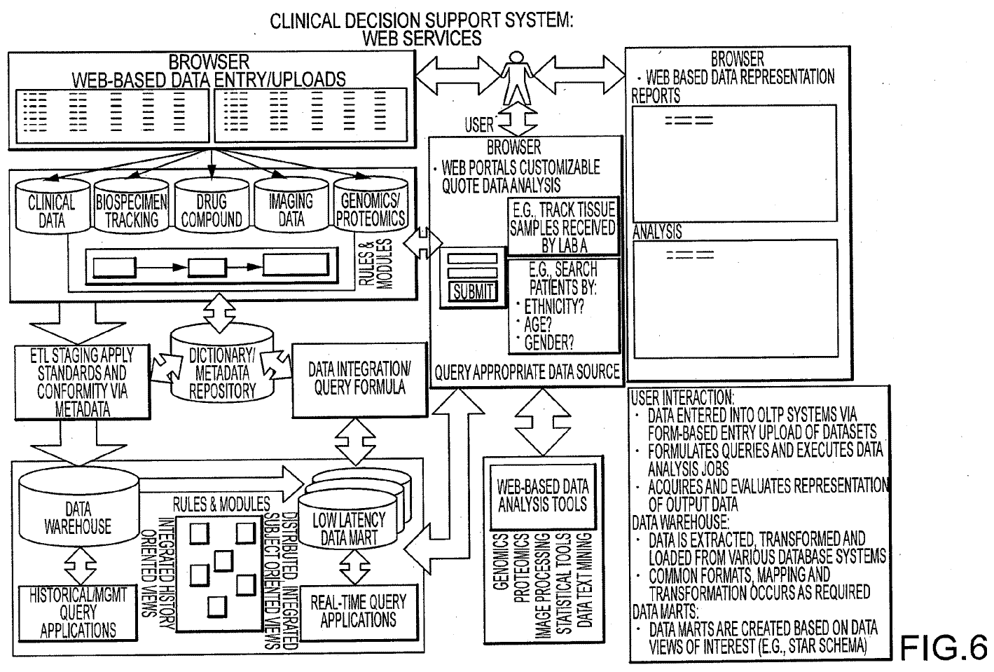

FIGS. 5-14 are flowcharts and diagrams illustrating various parts of an information-based personalized medicine drug discovery system and method in accordance with the present invention. FIG. 5 is a diagram showing an illustrative clinical decision support system of the information-based personalized medicine drug discovery system and method of the present invention. Data obtained through clinical research and clinical care such as clinical trial data, biomedical/molecular imaging data, genomics/proteomics/chemical library/literature/expert curation, biospecimen tracking/LIMS, family history/environmental records, and clinical data are collected and stored as databases and datamarts within a data warehouse. FIG. 6 is a diagram showing the flow of information through the clinical decision support system of the information-based personalized medicine drug discovery system and method of the present invention using web services. A user interacts with the system by entering data into the system via form-based entry/upload of data sets, formulating queries and executing data analysis jobs, and acquiring and evaluating representations of output data. The data warehouse in the web based system is where data is extracted, transformed, and loaded from various database systems. The data warehouse is also where common formats, mapping and transformation occurs. The web based system also includes datamarts which are created based on data views of interest.

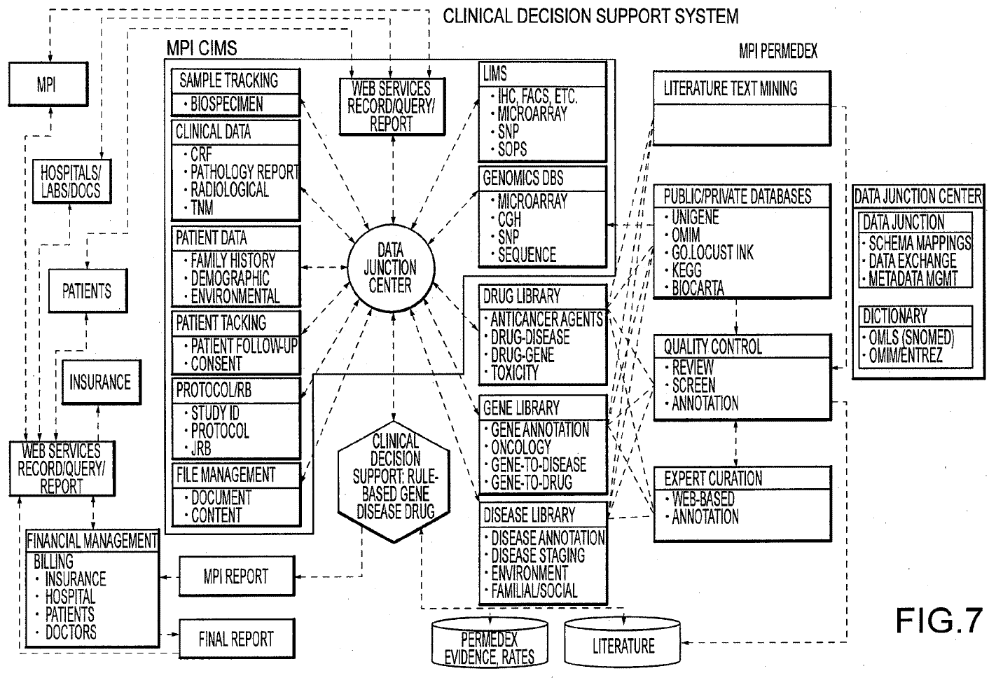

A flow chart of an illustrative clinical decision support system of the information-based personalized medicine drug discovery system and method of the present invention is shown in FIG. 7. The clinical information management system includes the laboratory information management system and the medical information contained in the data warehouses and databases includes medical information libraries, such as drug libraries, gene libraries, and disease libraries, in addition to literature text mining. Both the information management systems relating to particular patients and the medical information databases and data warehouses come together at a data junction center where diagnostic information and therapeutic options can be obtained. A financial management system may also be incorporated in the clinical decision support system of the information-based personalized medicine drug discovery system and method of the present invention.

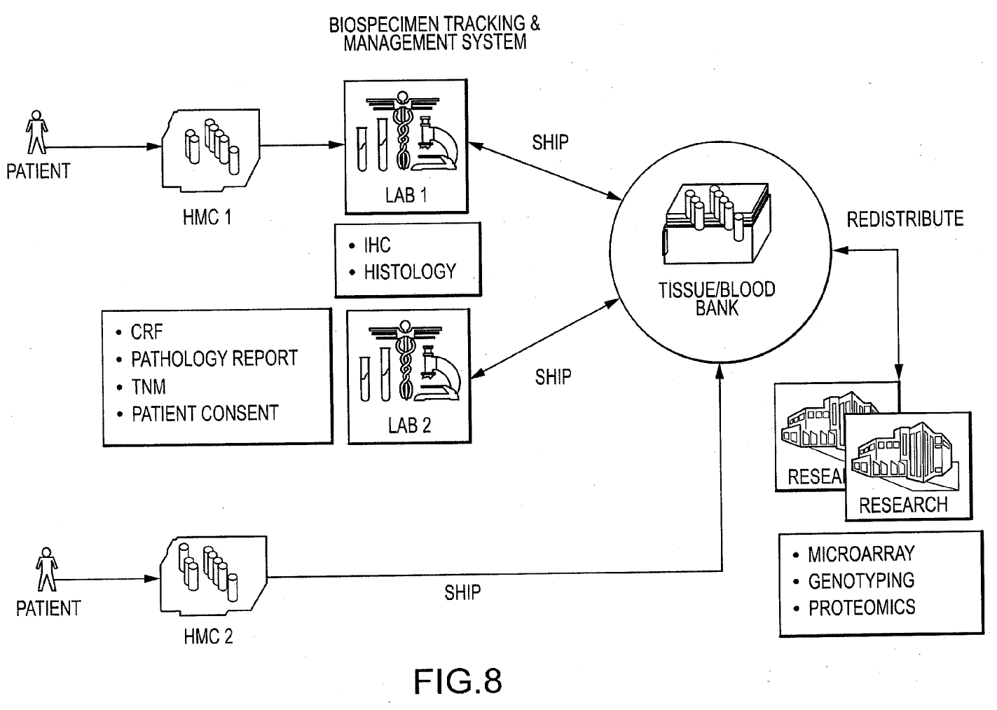

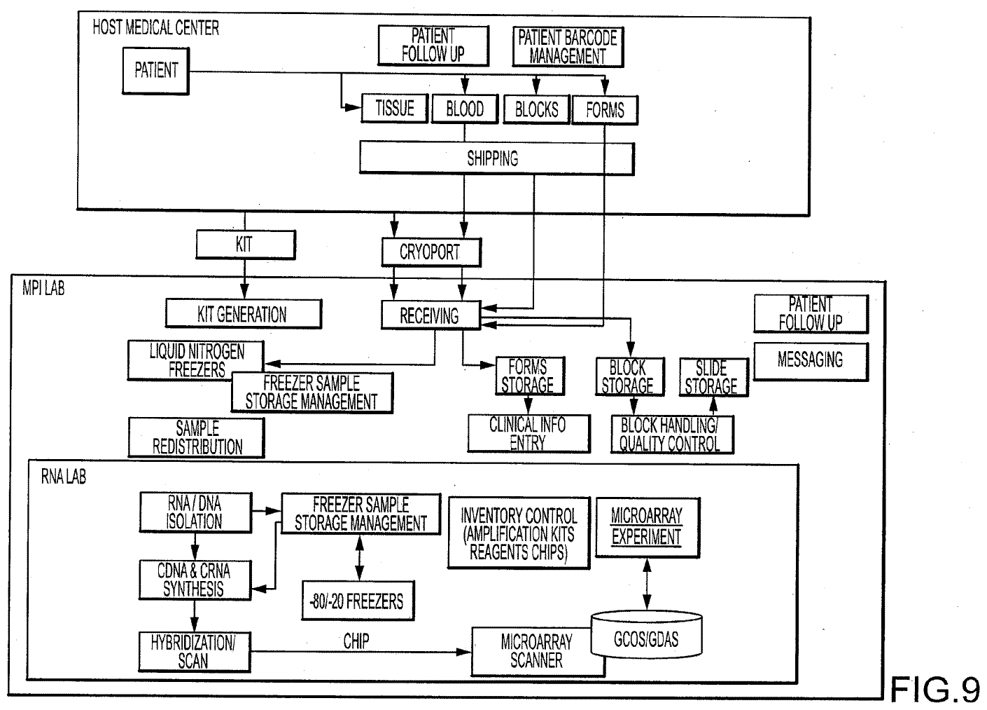

FIG. 8 is a diagram showing an illustrative biospecimen tracking and management system which may be used as part of the information-based personalized medicine drug discovery system and method of the present invention. FIG. 8 shows two host medical centers which forward specimens to a tissue/blood bank. The specimens may go through laboratory analysis prior to shipment. Research may also be conducted on the samples via micro array, genotyping, and proteomic analysis. This information can be redistributed to the tissue/blood bank. FIG. 9 depicts a flow chart of an illustrative biospecimen tracking and management system which may be used with the information-based personalized medicine drug discovery system and method of the present invention. The host medical center obtains samples from patients and then ships the patient samples to a molecular profiling laboratory which may also perform RNA and DNA isolation and analysis.

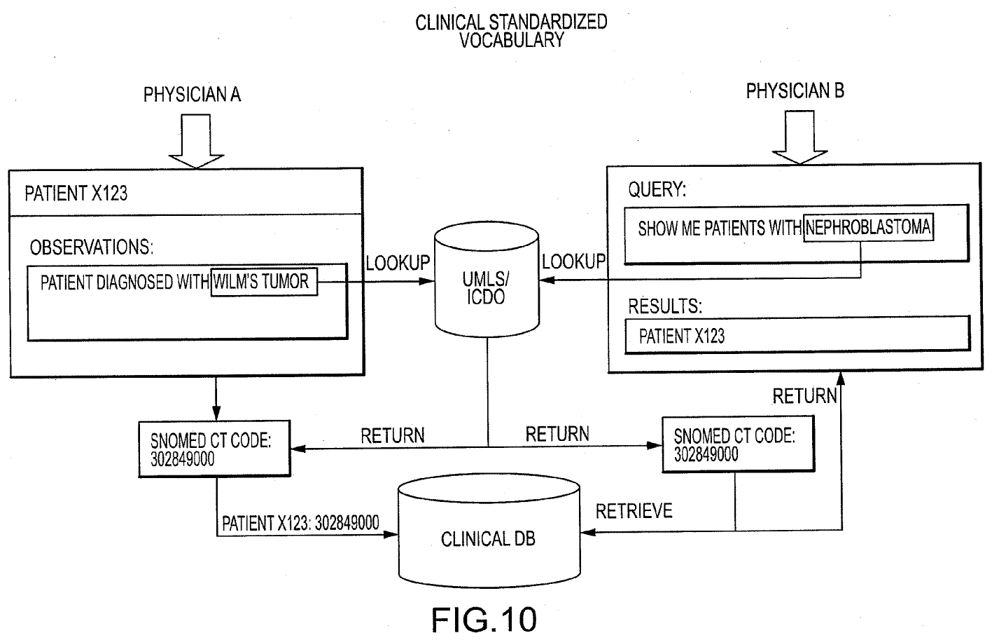

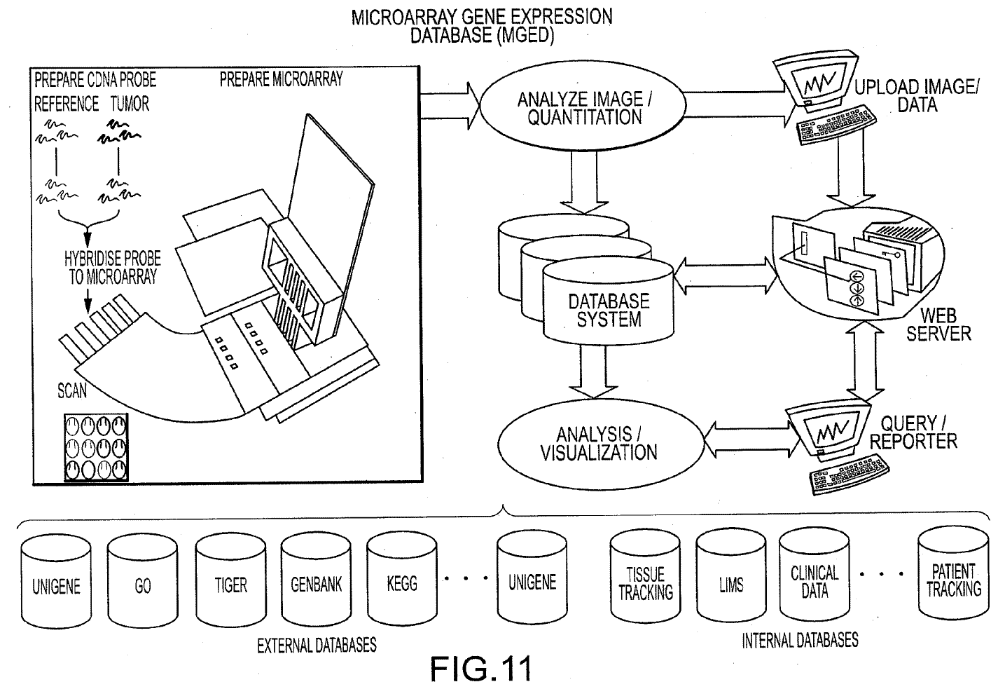

A diagram showing a method for maintaining a clinical standardized vocabulary for use with the information-based personalized medicine drug discovery system and method of the present invention is shown in FIG. 10. FIG. 10 illustrates how physician observations and patient information associated with one physician's patient may be made accessible to another physician to enable the other physician to use the data in making diagnostic and therapeutic decisions for their patients.

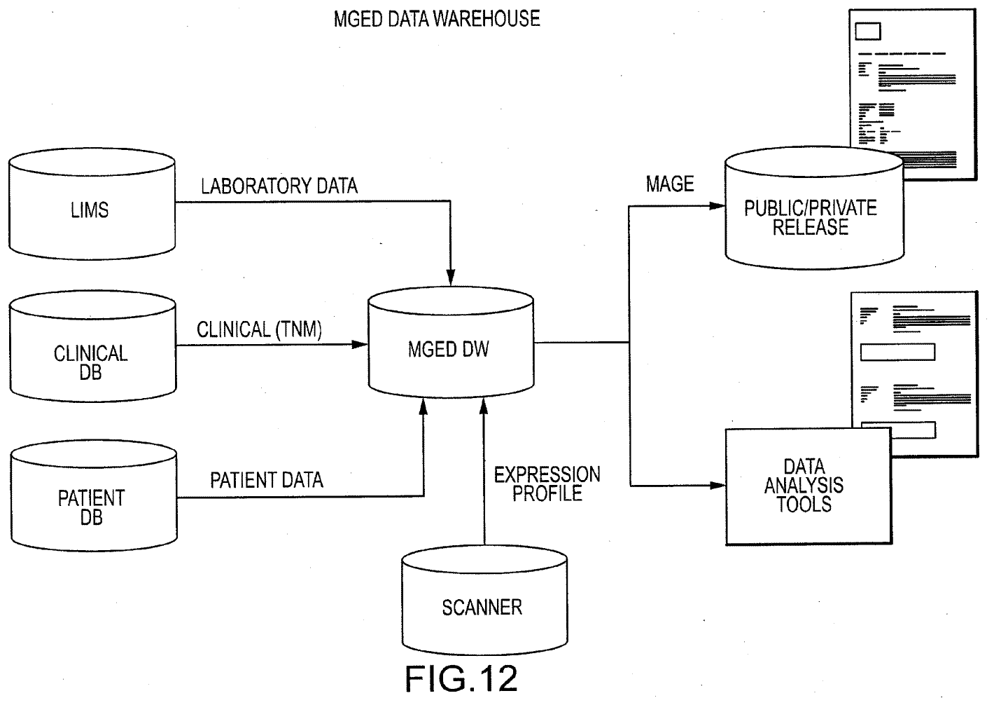

FIG. 11 shows a schematic of an illustrative microarray gene expression database which may be used as part of the information-based personalized medicine drug discovery system and method of the present invention. The micro array gene expression database includes both external databases and internal databases which can be accessed via the web based system. External databases may include, but are not limited to, UniGene, GO, TIGR, GenBank, KEGG. The internal databases may include, but are not limited to, tissue tracking, LIMS, clinical data, and patient tracking. FIG. 12 shows a diagram of an illustrative micro array gene expression database data warehouse which may be used as part of the information-based personalized medicine drug discovery system and method of the present invention. Laboratory data, clinical data, and patient data may all be housed in the micro array gene expression database data warehouse and the data may in turn be accessed by public/private release and used by data analysis tools.

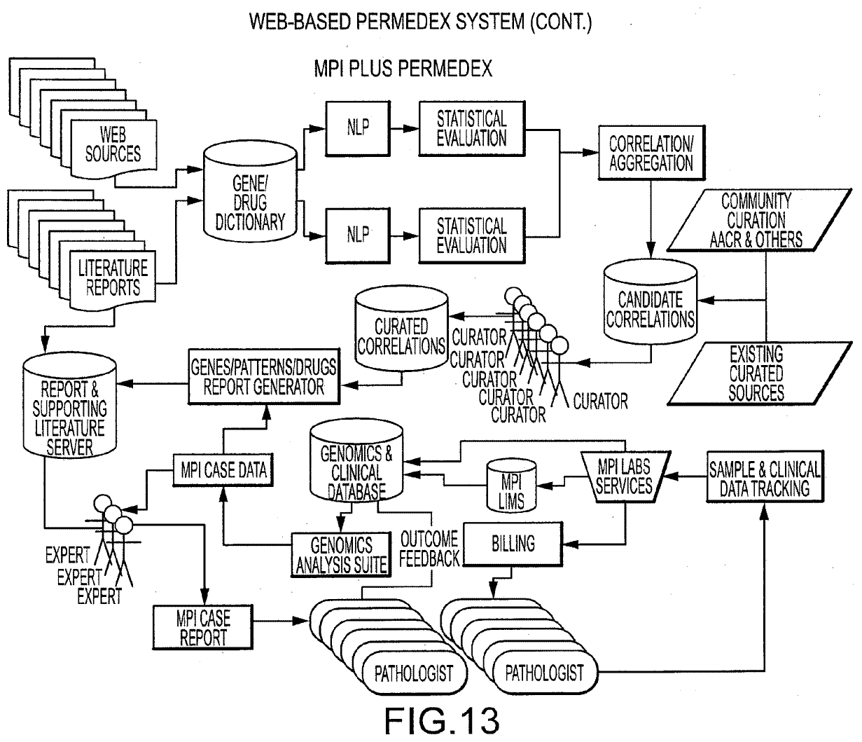

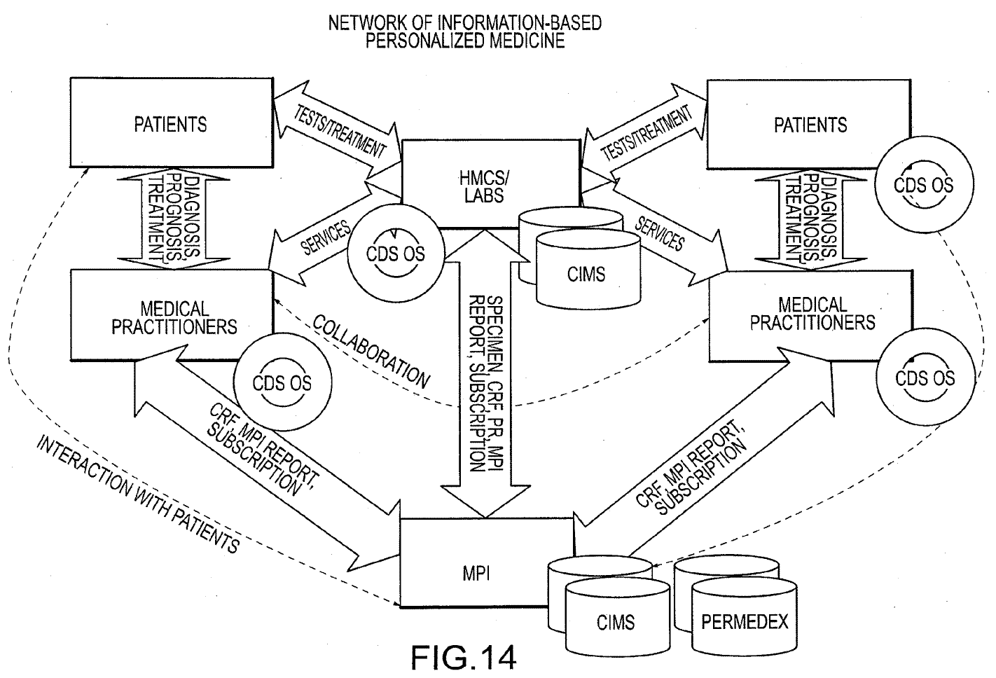

Another schematic showing the flow of information through an information-based personalized medicine drug discovery system and method of the present invention is shown in FIG. 13. Like FIG. 7, the schematic includes clinical information management, medical and literature information management, and financial management of the information-based personalized medicine drug discovery system and method of the present invention. FIG. 14 is a schematic showing an illustrative network of the information-based personalized medicine drug discovery system and method of the present invention. Patients, medical practitioners, host medical centers, and labs all share and exchange a variety of information in order to provide a patient with a proposed therapy or agent based on various identified targets.

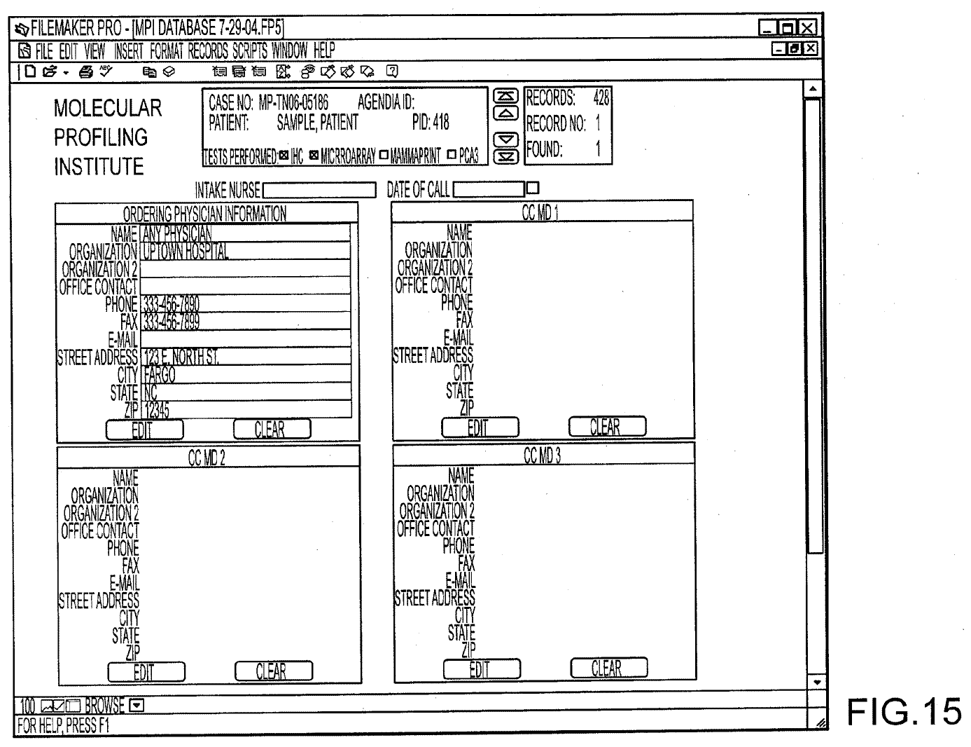

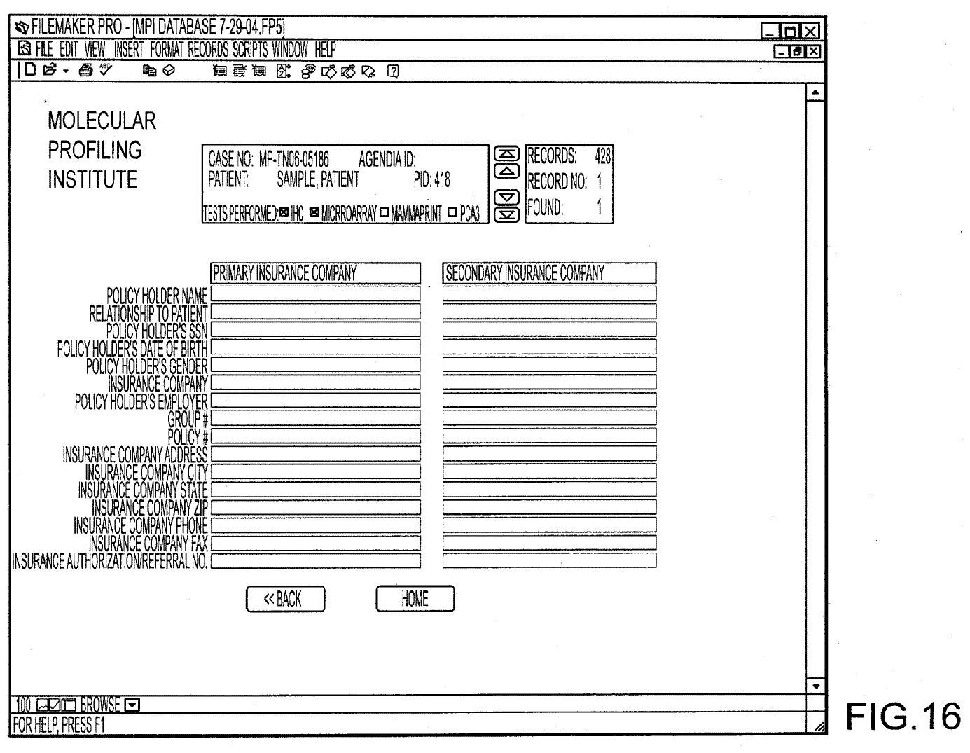

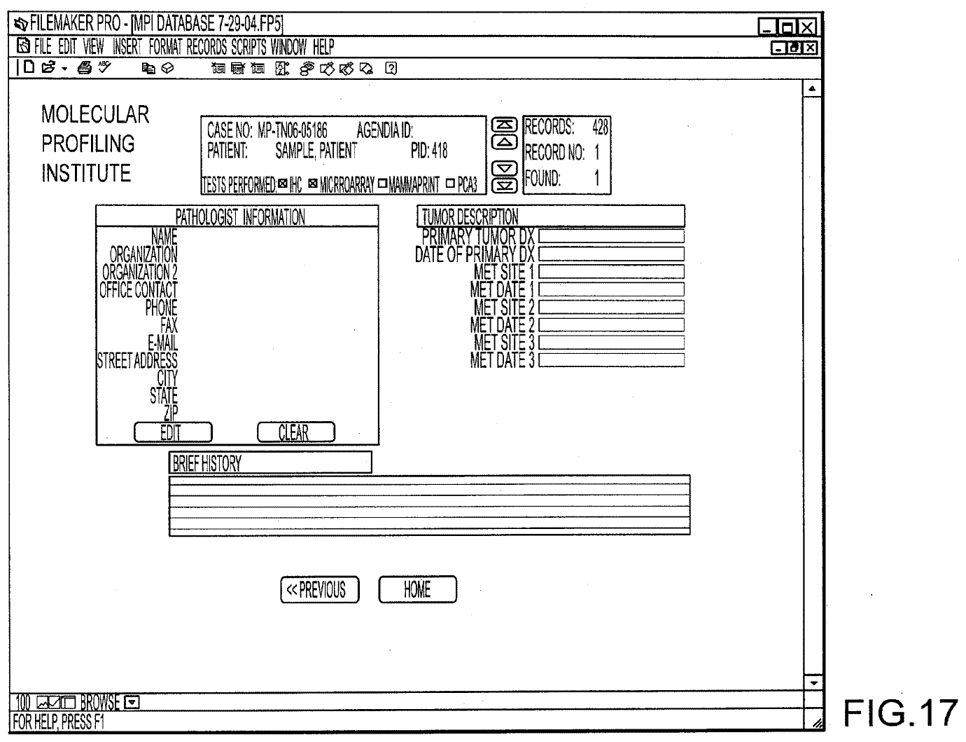

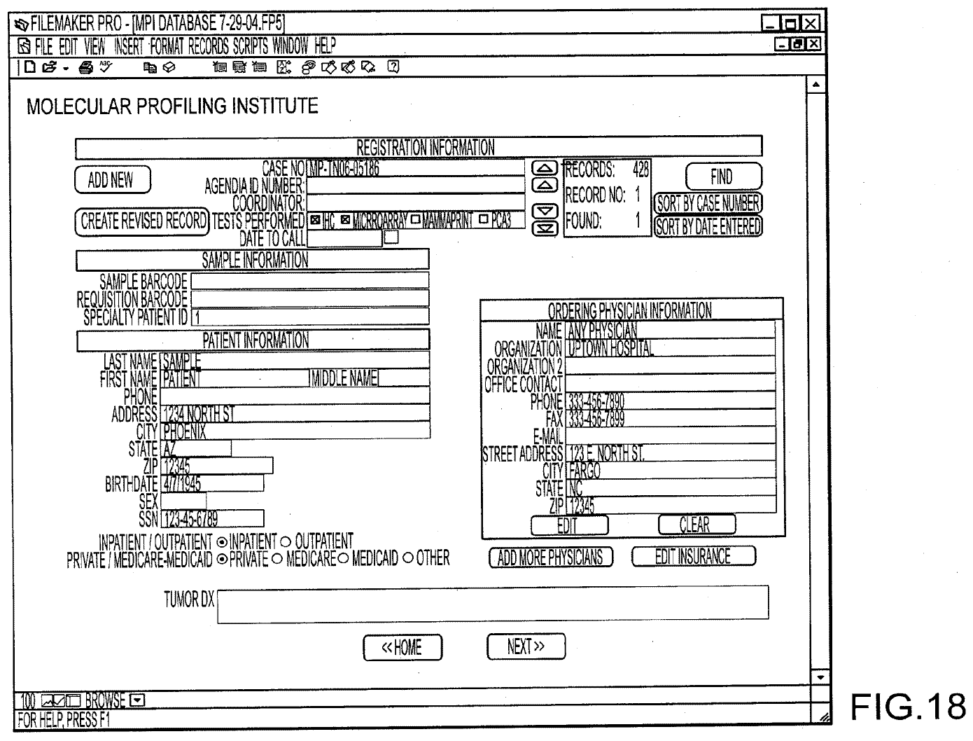

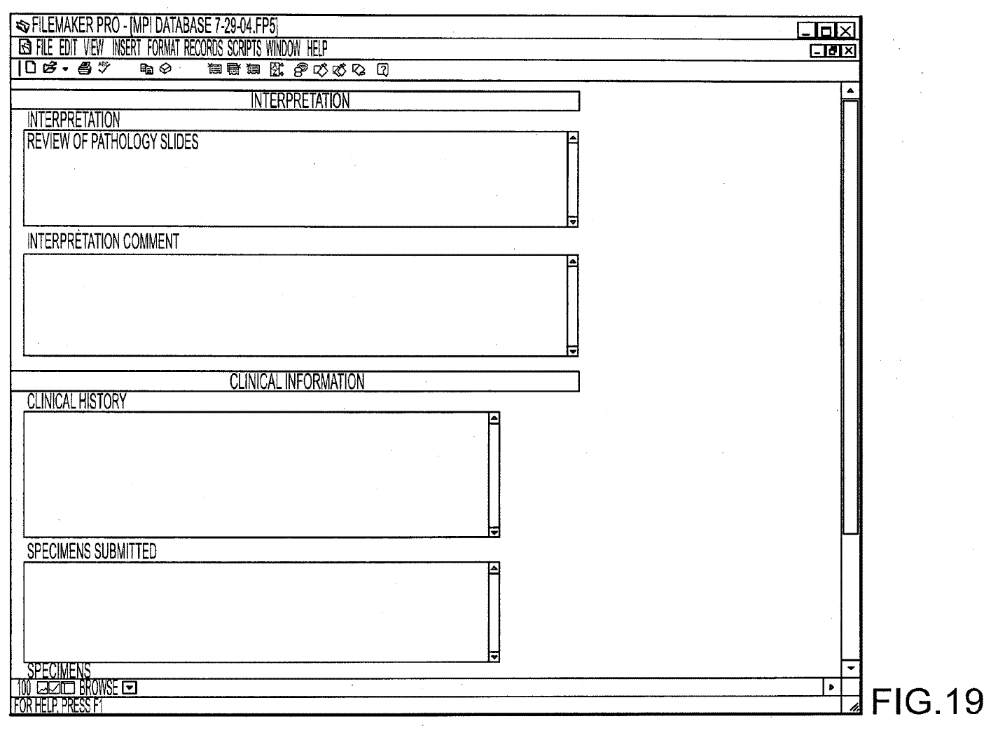

FIGS. 15-25 are computer screen print outs associated with various parts of the information-based personalized medicine drug discovery system and method shown in FIGS. 5-14. FIG. 15 and FIG. 16 show computer screens where physician information and insurance company information is entered on behalf of a client. FIG. 17, FIG. 18 and FIG. 19 show computer screens in which information can be entered for ordering analysis and tests on patient samples.

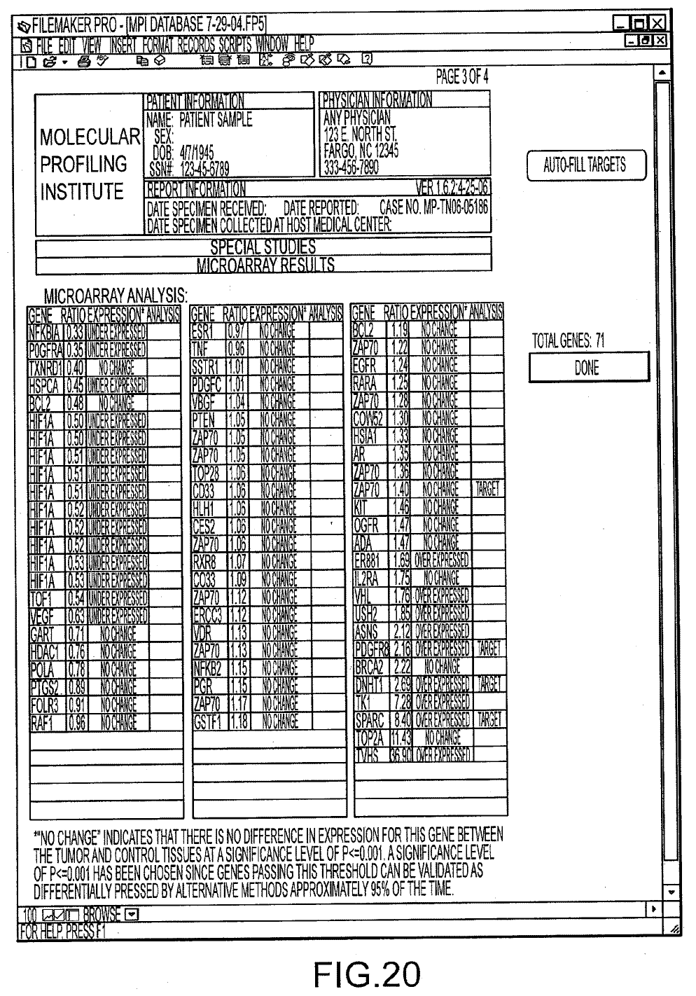

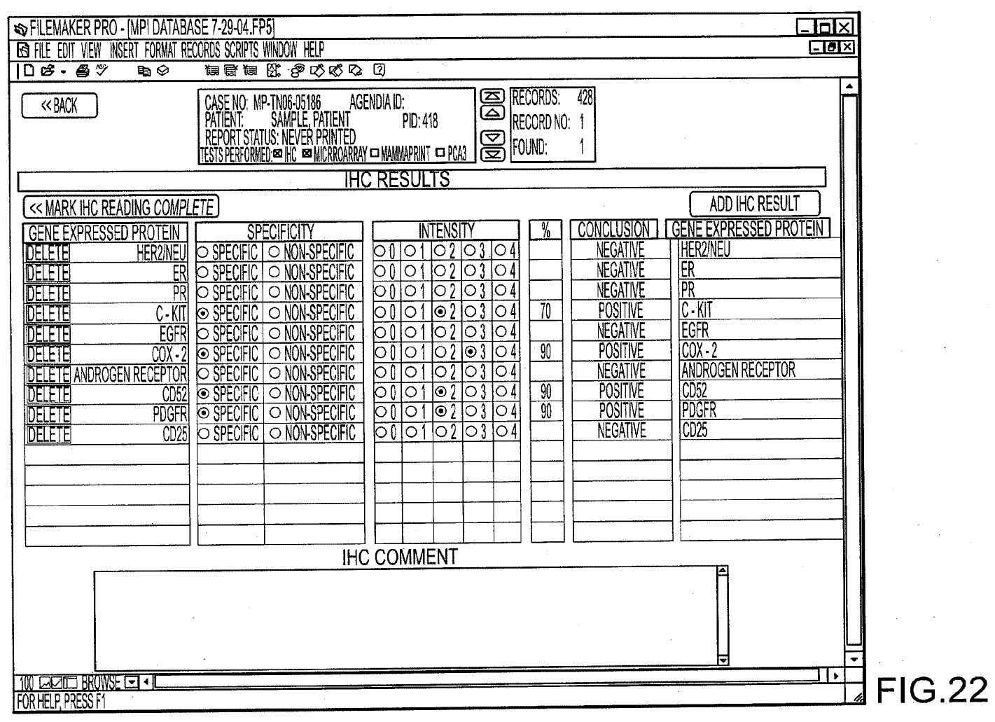

FIG. 20 is a computer screen showing micro array analysis results of specific genes tested with patient samples. This information and computer screen is similar to the information detailed in the patient profile report shown in FIG. 3C. FIG. 22 is a computer screen that shows immunohistochemistry test results for a particular patient for various genes. This information is similar to the information contained in the patient profile report shown in FIG. 3B.

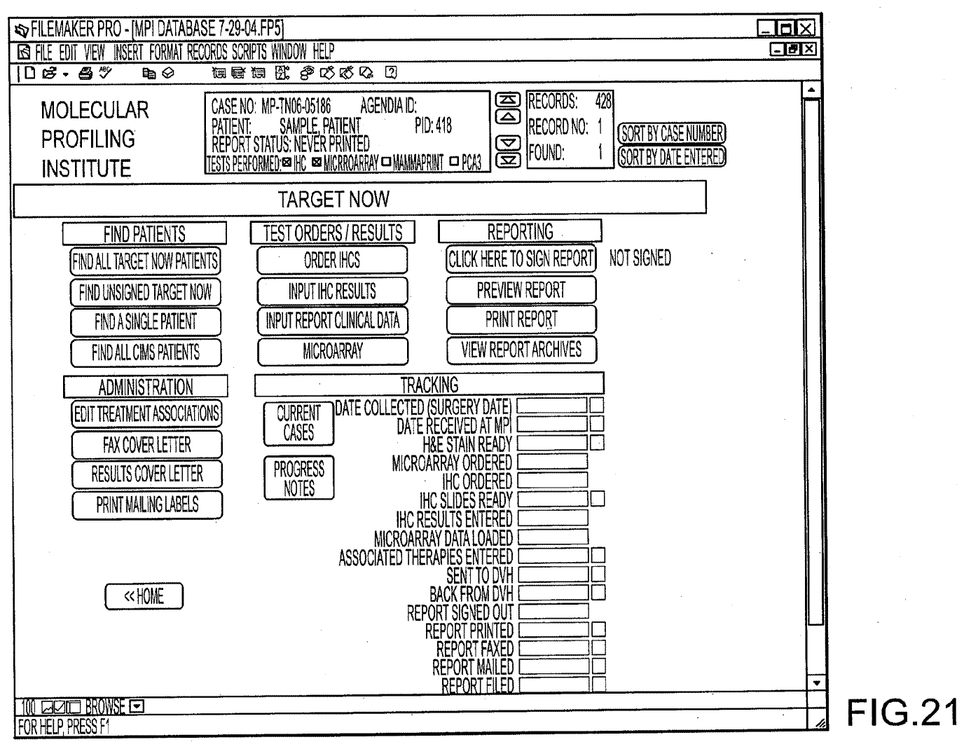

FIG. 21 is a computer screen showing selection options for finding particular patients, ordering tests and/or results, issuing patient reports, and tracking current cases/patients.

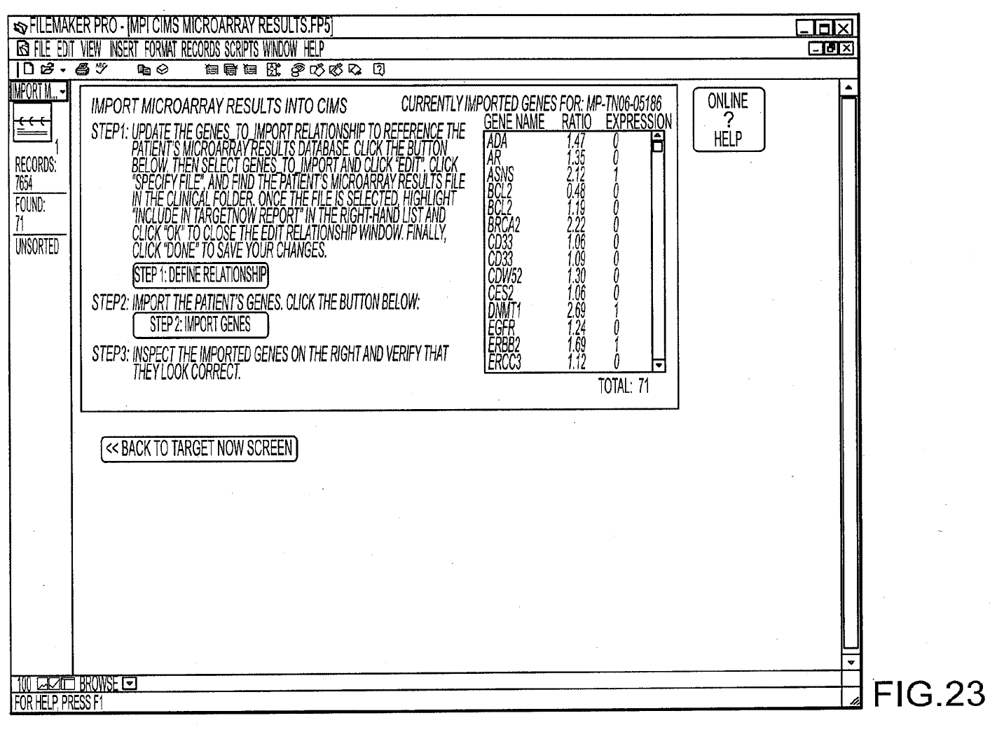

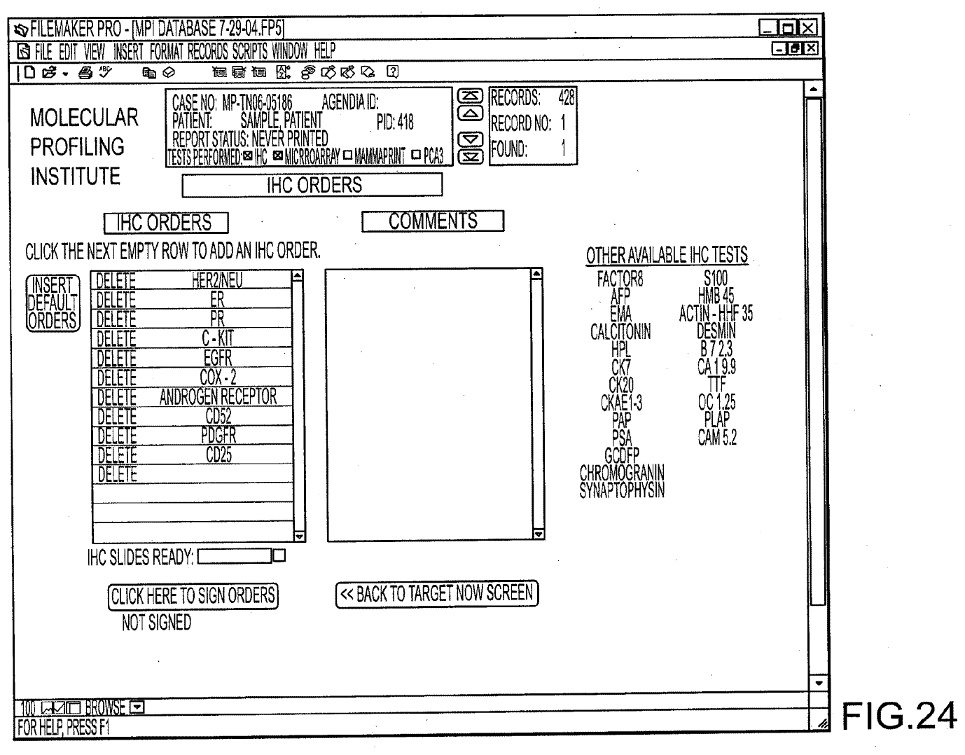

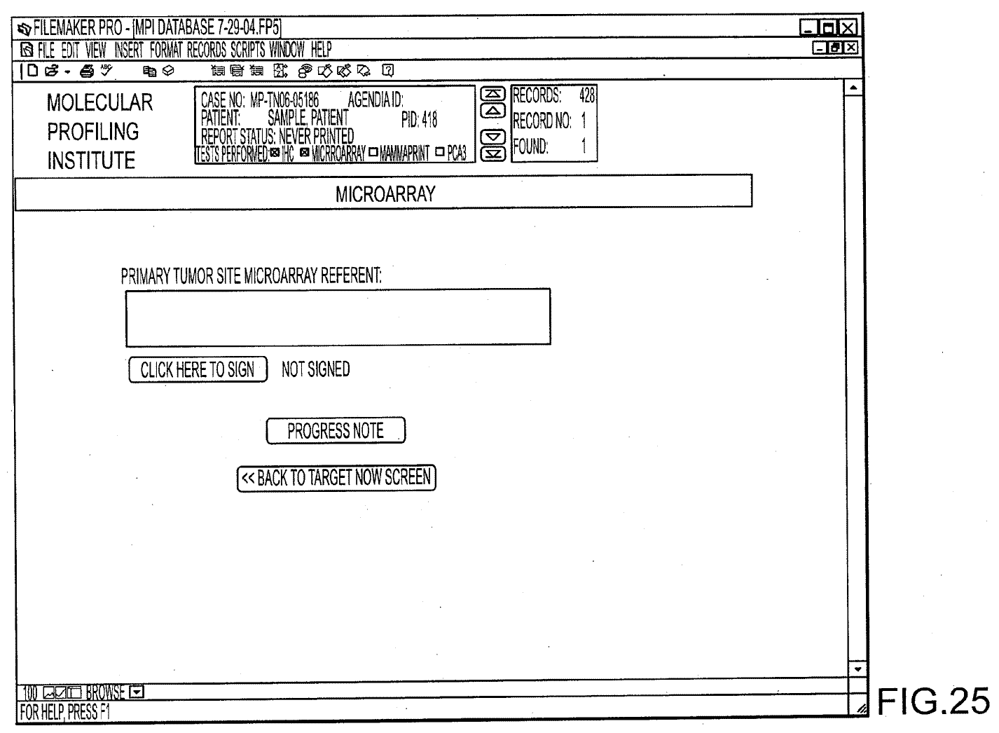

FIG. 23 is a computer screen which outlines some of the steps for creating a patient profile report as shown in FIGS. 3A through 3D. FIG. 24 shows a computer screen for ordering an immunohistochemistry test on a patient sample and FIG. 25 shows a computer screen for entering information regarding a primary tumor site for micro array analysis. It will be understood by those skilled in the art that any number and variety of computer screens may be used to enter the information necessary for using the information-based personalized medicine drug discovery system and method of the present invention and to obtain information resulting from using the information-based personalized medicine drug discovery system and method of the present invention.

The systems of the invention can be used to automate the steps of identifying a molecular profile to assess a cancer. In an aspect, the invention provides a method of generating a report comprising a molecular profile. The method comprises: performing a search on an electronic medium to obtain a data set, wherein the data set comprises a plurality of scientific publications corresponding to plurality of cancer biomarkers; and analyzing the data set to identify a rule set linking a characteristic of each of the plurality of cancer biomarkers with an expected benefit of a plurality of treatment options, thereby identifying the cancer biomarkers included within a molecular profile. The method can further comprise performing molecular profiling on a sample from a subject to assess the characteristic of each of the plurality of cancer biomarkers, and compiling a report comprising the assessed characteristics into a list, thereby generating a report that identifies a molecular profile for the sample. The report can further comprise a list describing the expected benefit of the plurality of treatment options based on the assessed characteristics, thereby identifying candidate treatment options for the subject. The sample from the subject may comprise cancer cells. The cancer can be any cancer disclosed herein or known in the art.

The characteristic of each of the plurality of cancer biomarkers can be any useful characteristic for molecular profiling as disclosed herein or known in the art. Such characteristics include without limitation mutations (point mutations, insertions, deletions, rearrangements, etc), epigenetic modifications, copy number, nucleic acid or protein expression levels, post-translational modifications, and the like.

In an embodiment, the method further comprises identifying a priority list as amongst said plurality of cancer biomarkers. The priority list can be sorted according to any appropriate priority criteria. In an embodiment, the priority list is sorted according to strength of evidence in the plurality of scientific publications linking the cancer biomarkers to the expected benefit. In another embodiment, the priority list is sorted according to strength of the expected benefit. In still another embodiment, the priority list is sorted according to strength of the expected benefit. One of skill will appreciate that the priority list can be sorted according to a combination of these or other appropriate priority criteria. The candidate treatment options can be sorted according to the priority list, thereby identifying a ranked list of treatment options for the subject.

The candidate treatment options can be categorized by expected benefit to the subject. For example, the candidate treatment options can categorized as those that are expected to provide benefit, those that are not expected to provide benefit, or those whose expected benefit cannot be determined.

The candidate treatment options can include regulatory approved and/or on-compendium treatments for the cancer. The candidate treatment options can include regulatory approved but off-label treatments for the cancer, such as a treatment that has been approved for a cancer of another lineage. The candidate treatment options can include treatments that are under development, such as in ongoing clinical trials. The report may identify treatments as approved, on- or off-compendium, in clinical trials, and the like.

In some embodiments, the method further comprises analyzing the data set to select a laboratory technique to assess the characteristics of the biomarkers, thereby designating a technique that can be used to assess the characteristic for each of the plurality of biomarkers. In other embodiments, the laboratory technique is chosen based on its applicability to assess the characteristic of each of the biomarkers. The laboratory techniques can be those disclosed herein, including without limitation FISH for gene copy number or mutation analysis, IHC for protein expression levels, RT-PCR for mutation or expression analysis, sequencing or fragment analysis for mutation analysis. Sequencing includes any useful sequencing method disclosed herein or known in the art, including without limitation Sanger sequencing, pyrosequencing, or next generation sequencing methods.

In a related aspect, the invention provides a method comprising: performing a search on an electronic medium to obtain a data set comprising a plurality of scientific publications corresponding to plurality of cancer biomarkers; analyzing the data set to select a method to assess a characteristic of each of the cancer biomarkers, thereby designating a method for characterizing each of the biomarkers; further analyzing the data set to select a rule set that identifies a priority list as amongst the biomarkers; performing tumor profiling on a tumor sample from a subject comprising the selected methods to determine the status of the characteristic of each of the biomarkers; and compiling the status in a report according to said priority list; thereby generating a report that identifies a tumor profile. Molecular Profiling Targets

The present invention provides methods and systems for analyzing diseased tissue using molecular profiling as previously described above. Because the methods rely on analysis of the characteristics of the tumor under analysis, the methods can be applied in for any tumor or any stage of disease, such an advanced stage of disease or a metastatic tumor of unknown origin. As described herein, a tumor or cancer sample is analyzed for molecular characteristics in order to predict or identify a candidate therapeutic treatment. The molecular characteristics can include the expression of genes or gene products, assessment of gene copy number, or mutational analysis. Any relevant determinable characteristic that can assist in prediction or identification of a candidate therapeutic can be included within the methods of the invention.

The biomarker patterns or biomarker signature sets can be determined for tumor types, diseased tissue types, or diseased cells including without limitation adipose, adrenal cortex, adrenal gland, adrenal gland—medulla, appendix, bladder, blood vessel, bone, bone cartilage, brain, breast, cartilage, cervix, colon, colon sigmoid, dendritic cells, skeletal muscle, endometrium, esophagus, fallopian tube, fibroblast, gallbladder, kidney, larynx, liver, lung, lymph node, melanocytes, mesothelial lining, myoepithelial cells, osteoblasts, ovary, pancreas, parotid, prostate, salivary gland, sinus tissue, skeletal muscle, skin, small intestine, smooth muscle, stomach, synovium, joint lining tissue, tendon, testis, thymus, thyroid, uterus, and uterus corpus.

The methods of the present invention can be used for selecting a treatment of any cancer or tumor type, including but not limited to breast cancer (including HER2+ breast cancer, HER2− breast cancer, ER/PR+, HER2− breast cancer, or triple negative breast cancer), pancreatic cancer, cancer of the colon and/or rectum, leukemia, skin cancer, bone cancer, prostate cancer, liver cancer, lung cancer, brain cancer, cancer of the larynx, gallbladder, parathyroid, thyroid, adrenal, neural tissue, head and neck, stomach, bronchi, kidneys, basal cell carcinoma, squamous cell carcinoma of both ulcerating and papillary type, metastatic skin carcinoma, osteo sarcoma, Ewing's sarcoma, veticulum cell sarcoma, myeloma, giant cell tumor, small-cell lung tumor, islet cell carcinoma, primary brain tumor, acute and chronic lymphocytic and granulocytic tumors, hairy-cell tumor, adenoma, hyperplasia, medullary carcinoma, pheochromocytoma, mucosal neuroma, intestinal ganglioneuroma, hyperplastic corneal nerve tumor, marfanoid habitus tumor, Wilm's tumor, seminoma, ovarian tumor, leiomyoma, cervical dysplasia and in situ carcinoma, neuroblastoma, retinoblastoma, soft tissue sarcoma, malignant carcinoid, topical skin lesion, mycosis fungoides, rhabdomyosarcoma, Kaposi's sarcoma, osteogenic and other sarcoma, malignant hypercalcemia, renal cell tumor, polycythermia vera, adenocarcinoma, glioblastoma multiforma, leukemias, lymphomas, malignant melanomas, and epidermoid carcinomas. The cancer or tumor can comprise, without limitation, a carcinoma, a sarcoma, a lymphoma or leukemia, a germ cell tumor, a blastoma, or other cancers. Carcinomas that can be assessed using the subject methods include without limitation epithelial neoplasms, squamous cell neoplasms, squamous cell carcinoma, basal cell neoplasms basal cell carcinoma, transitional cell papillomas and carcinomas, adenomas and adenocarcinomas (glands), adenoma, adenocarcinoma, linitis plastica insulinoma, glucagonoma, gastrinoma, vipoma, cholangiocarcinoma, hepatocellular carcinoma, adenoid cystic carcinoma, carcinoid tumor of appendix, prolactinoma, oncocytoma, hurthle cell adenoma, renal cell carcinoma, grawitz tumor, multiple endocrine adenomas, endometrioid adenoma, adnexal and skin appendage neoplasms, mucoepidermoid neoplasms, cystic, mucinous and serous neoplasms, cystadenoma, pseudomyxoma peritonei, ductal, lobular and medullary neoplasms, acinar cell neoplasms, complex epithelial neoplasms, warthin's tumor, thymoma, specialized gonadal neoplasms, sex cord stromal tumor, thecoma, granulosa cell tumor, arrhenoblastoma, sertoli leydig cell tumor, glomus tumors, paraganglioma, pheochromocytoma, glomus tumor, nevi and melanomas, melanocytic nevus, malignant melanoma, melanoma, nodular melanoma, dysplastic nevus, lentigo maligna melanoma, superficial spreading melanoma, and malignant acral lentiginous melanoma. Sarcoma that can be assessed using the subject methods include without limitation Askin's tumor, botryodies, chondrosarcoma, Ewing's sarcoma, malignant hemangio endothelioma, malignant schwannoma, osteosarcoma, soft tissue sarcomas including: alveolar soft part sarcoma, angiosarcoma, cystosarcoma phyllodes, dermatofibrosarcoma, desmoid tumor, desmoplastic small round cell tumor, epithelioid sarcoma, extraskeletal chondrosarcoma, extraskeletal osteosarcoma, fibrosarcoma, hemangiopericytoma, hemangiosarcoma, kaposi's sarcoma, leiomyosarcoma, liposarcoma, lymphangiosarcoma, lymphosarcoma, malignant fibrous histiocytoma, neurofibrosarcoma, rhabdomyosarcoma, and synovialsarcoma. Lymphoma and leukemia that can be assessed using the subject methods include without limitation chronic lymphocytic leukemia/small lymphocytic lymphoma, B-cell prolymphocytic leukemia, lymphoplasmacytic lymphoma (such as waldenström macroglobulinemia), splenic marginal zone lymphoma, plasma cell myeloma, plasmacytoma, monoclonal immunoglobulin deposition diseases, heavy chain diseases, extranodal marginal zone B cell lymphoma, also called malt lymphoma, nodal marginal zone B cell lymphoma (nmzl), follicular lymphoma, mantle cell lymphoma, diffuse large B cell lymphoma, mediastinal (thymic) large B cell lymphoma, intravascular large B cell lymphoma, primary effusion lymphoma, burkitt lymphoma/leukemia, T cell prolymphocytic leukemia, T cell large granular lymphocytic leukemia, aggressive NK cell leukemia, adult T cell leukemia/lymphoma, extranodal NK/T cell lymphoma, nasal type, enteropathy-type T cell lymphoma, hepatosplenic T cell lymphoma, blastic NK cell lymphoma, mycosis fungoides/sezary syndrome, primary cutaneous CD30-positive T cell lymphoproliferative disorders, primary cutaneous anaplastic large cell lymphoma, lymphomatoid papulosis, angioimmunoblastic T cell lymphoma, peripheral T cell lymphoma, unspecified, anaplastic large cell lymphoma, classical Hodgkin lymphomas (nodular sclerosis, mixed cellularity, lymphocyte-rich, lymphocyte depleted or not depleted), and nodular lymphocyte-predominant Hodgkin lymphoma. Germ cell tumors that can be assessed using the subject methods include without limitation germinoma, dysgerminoma, seminoma, nongerminomatous germ cell tumor, embryonal carcinoma, endodermal sinus turmor, choriocarcinoma, teratoma, polyembryoma, and gonadoblastoma. Blastoma includes without limitation nephroblastoma, medulloblastoma, and retinoblastoma. Other cancers include without limitation labial carcinoma, larynx carcinoma, hypopharynx carcinoma, tongue carcinoma, salivary gland carcinoma, gastric carcinoma, adenocarcinoma, thyroid cancer (medullary and papillary thyroid carcinoma), renal carcinoma, kidney parenchyma carcinoma, cervix carcinoma, uterine corpus carcinoma, endometrium carcinoma, chorion carcinoma, testis carcinoma, urinary carcinoma, melanoma, brain tumors such as glioblastoma, astrocytoma, meningioma, medulloblastoma and peripheral neuroectodermal tumors, gall bladder carcinoma, bronchial carcinoma, multiple myeloma, basalioma, teratoma, retinoblastoma, choroidea melanoma, seminoma, rhabdomyosarcoma, craniopharyngeoma, osteosarcoma, chondrosarcoma, myosarcoma, liposarcoma, fibrosarcoma, Ewing sarcoma, and plasmocytoma.

In an embodiment, the cancer may be a acute myeloid leukemia (AML), breast carcinoma, cholangiocarcinoma, colorectal adenocarcinoma, extrahepatic bile duct adenocarcinoma, female genital tract malignancy, gastric adenocarcinoma, gastroesophageal adenocarcinoma, gastrointestinal stromal tumors (GIST), glioblastoma, head and neck squamous carcinoma, leukemia, liver hepatocellular carcinoma, low grade glioma, lung bronchioloalveolar carcinoma (BAC), lung non-small cell lung cancer (NSCLC), lung small cell cancer (SCLC), lymphoma, male genital tract malignancy, malignant solitary fibrous tumor of the pleura (MSFT), melanoma, multiple myeloma, neuroendocrine tumor, nodal diffuse large B-cell lymphoma, non epithelial ovarian cancer (non-EOC), ovarian surface epithelial carcinoma, pancreatic adenocarcinoma, pituitary carcinomas, oligodendroglioma, prostatic adenocarcinoma, retroperitoneal or peritoneal carcinoma, retroperitoneal or peritoneal sarcoma, small intestinal malignancy, soft tissue tumor, thymic carcinoma, thyroid carcinoma, or uveal melanoma.

In a further embodiment, the cancer may be a lung cancer including non-small cell lung cancer and small cell lung cancer (including small cell carcinoma (oat cell cancer), mixed small cell/large cell carcinoma, and combined small cell carcinoma), colon cancer, breast cancer, prostate cancer, liver cancer, pancreas cancer, brain cancer, kidney cancer, ovarian cancer, stomach cancer, skin cancer, bone cancer, gastric cancer, breast cancer, pancreatic cancer, glioma, glioblastoma, hepatocellular carcinoma, papillary renal carcinoma, head and neck squamous cell carcinoma, leukemia, lymphoma, myeloma, or a solid tumor.

In embodiments, the cancer comprises an acute lymphoblastic leukemia; acute myeloid leukemia; adrenocortical carcinoma; AIDS-related cancers; AIDS-related lymphoma; anal cancer; appendix cancer; astrocytomas; atypical teratoid/rhabdoid tumor; basal cell carcinoma; bladder cancer; brain stem glioma; brain tumor (including brain stem glioma, central nervous system atypical teratoid/rhabdoid tumor, central nervous system embryonal tumors, astrocytomas, craniopharyngioma, ependymoblastoma, ependymoma, medulloblastoma, medulloepithelioma, pineal parenchymal tumors of intermediate differentiation, supratentorial primitive neuroectodermal tumors and pineoblastoma); breast cancer; bronchial tumors; Burkitt lymphoma; cancer of unknown primary site; carcinoid tumor; carcinoma of unknown primary site; central nervous system atypical teratoid/rhabdoid tumor; central nervous system embryonal tumors; cervical cancer; childhood cancers; chordoma; chronic lymphocytic leukemia; chronic myelogenous leukemia; chronic myeloproliferative disorders; colon cancer; colorectal cancer; craniopharyngioma; cutaneous T-cell lymphoma; endocrine pancreas islet cell tumors; endometrial cancer; ependymoblastoma; ependymoma; esophageal cancer; esthesioneuroblastoma; Ewing sarcoma; extracranial germ cell tumor; extragonadal germ cell tumor; extrahepatic bile duct cancer; gallbladder cancer; gastric (stomach) cancer; gastrointestinal carcinoid tumor; gastrointestinal stromal cell tumor; gastrointestinal stromal tumor (GIST); gestational trophoblastic tumor; glioma; hairy cell leukemia; head and neck cancer; heart cancer; Hodgkin lymphoma; hypopharyngeal cancer; intraocular melanoma; islet cell tumors; Kaposi sarcoma; kidney cancer; Langerhans cell histiocytosis; laryngeal cancer; lip cancer; liver cancer; malignant fibrous histiocytoma bone cancer; medulloblastoma; medulloepithelioma; melanoma; Merkel cell carcinoma; Merkel cell skin carcinoma; mesothelioma; metastatic squamous neck cancer with occult primary; micropapillary urothelial carcinoma; mouth cancer; multiple endocrine neoplasia syndromes; multiple myeloma; multiple myeloma/plasma cell neoplasm; mycosis fungoides; myelodysplastic syndromes; myeloproliferative neoplasms; nasal cavity cancer; nasopharyngeal cancer; neuroblastoma; Non-Hodgkin lymphoma; nonmelanoma skin cancer; non-small cell lung cancer; oral cancer; oral cavity cancer; oropharyngeal cancer; osteosarcoma; other brain and spinal cord tumors; ovarian cancer; ovarian epithelial cancer; ovarian germ cell tumor; ovarian low malignant potential tumor; pancreatic cancer; papillomatosis; paranasal sinus cancer; parathyroid cancer; pelvic cancer; penile cancer; pharyngeal cancer; pineal parenchymal tumors of intermediate differentiation; pineoblastoma; pituitary tumor; plasma cell neoplasm/multiple myeloma; pleuropulmonary blastoma; primary central nervous system (CNS) lymphoma; primary hepatocellular liver cancer; prostate cancer; rectal cancer; renal cancer; renal cell (kidney) cancer; renal cell cancer; respiratory tract cancer; retinoblastoma; rhabdomyosarcoma; salivary gland cancer; Sdzary syndrome; small cell lung cancer; small intestine cancer; soft tissue sarcoma; squamous cell carcinoma; squamous neck cancer; stomach (gastric) cancer; supratentorial primitive neuroectodermal tumors; T-cell lymphoma; testicular cancer; throat cancer; thymic carcinoma; thymoma; thyroid cancer; transitional cell cancer; transitional cell cancer of the renal pelvis and ureter; trophoblastic tumor; ureter cancer; urethral cancer; uterine cancer; uterine sarcoma; vaginal cancer; vulvar cancer; Waldenström macroglobulinemia; or Wilm's tumor.

The methods of the invention can be used to determine biomarker patterns or biomarker signature sets in a number of tumor types, diseased tissue types, or diseased cells including accessory, sinuses, middle and inner ear, adrenal glands, appendix, hematopoietic system, bones and joints, spinal cord, breast, cerebellum, cervix uteri, connective and soft tissue, corpus uteri, esophagus, eye, nose, eyeball, fallopian tube, extrahepatic bile ducts, other mouth, intrahepatic bile ducts, kidney, appendix-colon, larynx, lip, liver, lung and bronchus, lymph nodes, cerebral, spinal, nasal cartilage, excl, retina, eye, nos, oropharynx, other endocrine glands, other female genital, ovary, pancreas, penis and scrotum, pituitary gland, pleura, prostate gland, rectum renal pelvis, ureter, peritonem, salivary gland, skin, small intestine, stomach, testis, thymus, thyroid gland, tongue, unknown, urinary bladder, uterus, nos, vagina & labia, and vulva,nos.

In some embodiments, the molecular profiling methods are used to identify a treatment for a cancer of unknown primary (CUP). Approximately 40,000 CUP cases are reported annually in the US. Most of these are metastatic and/or poorly differentiated tumors. Because molecular profiling can identify a candidate treatment depending only upon the diseased sample, the methods of the invention can be used in the CUP setting. Moreover, molecular profiling can be used to create signatures of known tumors, which can then be used to classify a CUP and identify its origin. In an aspect, the invention provides a method of identifying the origin of a CUP, the method comprising performing molecular profiling on a panel of diseased samples to determine a panel of molecular profiles that correlate with the origin of each diseased sample, performing molecular profiling on a CUP sample, and correlating the molecular profile of the CUP sample with the molecular profiling of the panel of diseased samples, thereby identifying the origin of the CUP sample. The identification of the origin of the CUP sample can be made by matching the molecular profile of the CUP sample with the molecular profiles that correlate most closely from the panel of disease samples.

The biomarker patterns or biomarker signature sets of the cancer or tumor can be used to determine a therapeutic agent or therapeutic protocol that is capable of interacting with the biomarker pattern or signature set. For example, with advanced breast cancer, immunohistochemistry analysis can be used to determine one or more proteins that are overexpressed. Accordingly, a biomarker pattern or biomarker signature set can be identified for advanced stage breast cancer and a therapeutic agent or therapeutic protocol can be identified with predicted benefit (or lack thereof) for the patient.

The biomarker patterns and/or biomarker signature sets can comprise pluralities of biomarkers. In yet other embodiments, the biomarker patterns or signature sets can comprise at least 2, 3, 4, 5, 6, 7, 8, 9, or 10 biomarkers. In some embodiments, the biomarker signature sets or biomarker patterns can comprise at least 15, 20, 30, 40, 50, or 60 biomarkers. In some embodiments, the biomarker signature sets or biomarker patterns can comprise at least 70, 80, 90, 100, 200, 300, 400, 500, 600, 700, 800, 900, 1000, 2000, 3000, 4000, 5000, 6000, 7000, 8000, 9000, 10,000, 15,000, 20,000, 25,000, 30,000, 35,000, 40,000, 45,000 or 50,000 biomarkers. Analysis of the one or more biomarkers can be by one or more methods. For example, analysis of 2 biomarkers can be performed using sequence analysis. Alternatively, one biomarker may be analyzed by IHC and another by sequencing. Any such combinations of useful methods and biomarkers are contemplated herein.

As described herein, the molecular profiling of one or more targets can be used to determine or identify a therapeutic for an individual. For example, the expression level of one or more biomarkers can be used to determine or identify a therapeutic for an individual. The one or more biomarkers, such as those disclosed herein, can be used to form a biomarker pattern or biomarker signature set, which is used to identify a therapeutic for an individual. In some embodiments, the therapeutic identified is one that the individual has not previously been treated with. For example, a reference biomarker pattern has been established for a particular therapeutic, such that individuals with the reference biomarker pattern will be responsive to that therapeutic. An individual with a biomarker pattern that differs from the reference, for example the expression of a gene in the biomarker pattern is changed or different from that of the reference, would not be administered that therapeutic. In another example, an individual exhibiting a biomarker pattern that is the same or substantially the same as the reference is advised to be treated with that therapeutic. In some embodiments, the individual has not previously been treated with that therapeutic and thus a new therapeutic has been identified for the individual.

Molecular profiling according to the invention can take on a biomarker-centric or a therapeutic-centric point of view. Although the approaches are not mutually exclusive, the biomarker-centric approach focuses on sets of biomarkers that are expected to be informative for a tumor of a given tumor lineage, whereas the therapeutic-centric point approach identifies candidate therapeutics using biomarker panels that are lineage independent. In a biomarker-centric view, panels of specific biomarkers are run on different tumor types. This approach provides a method of identifying a candidate therapeutic by collecting a sample from a subject with a cancer of known origin, and performing molecular profiling on the cancer for specific biomarkers depending on the origin of the cancer. The molecular profiling can be performed using any of the various techniques disclosed herein. As an example, biomarker panels may include those for breast cancer, ovarian cancer, colorectal cancer, lung cancer, and a profile to run on any cancer. See e.g., Table 5 for marker profiles that can be assessed for various cancer lineages. Markers can be assessed using various techniques such as sequencing approaches (NGS, pyrosequencing, etc), ISH (e.g., FISH/CISH), and for protein expression, e.g., using IHC. The candidate therapeutic can be selected based on the molecular profiling results according to the subject methods. A potential advantage to the bio-marker centric approach is only performing assays that are most likely to yield informative results in a given lineage. Another potential advantage is that this approach can focus on identifying therapeutics conventionally used to treat cancers of the specific lineage. In a therapeutic-centric approach, the biomarkers assessed are not dependent on the origin of the tumor. Rather, this approach provides a method of identifying a candidate therapeutic by collecting a sample from a subject with any given cancer, and performing molecular profiling on the cancer for a panel of biomarkers without regards to the origin of the cancer. The molecular profiling can be performed using any of the various techniques disclosed herein, e.g., such as described above. The candidate therapeutic is selected based on the molecular profiling results according to the subject methods. A potential advantage to the therapeutic-marker centric approach is that the most promising therapeutics are identified only taking into account the molecular characteristics of the tumor itself. Another advantage is that the method can be preferred for a cancer of unidentified primary origin (CUP). In some embodiments, a hybrid of biomarker-centric and therapeutic-centric points of view is used to identify a candidate therapeutic. This method comprises identifying a candidate therapeutic by collecting a sample from a subject with a cancer of known origin, and performing molecular profiling on the cancer for a comprehensive panel of biomarkers, wherein a portion of the markers assessed depend on the origin of the cancer. For example, consider a breast cancer. A comprehensive biomarker panel may be run on the breast cancer, e.g., that for any solid tumor as described herein, but additional sequencing analysis is performed on one or more additional markers, e.g., BRCA1 or any other marker with mutations informative for theranosis or prognosis of the breast cancer. Theranosis can be used to refer to the likely efficacy of a therapeutic treatment. Prognosis refers to the likely outcome of an illness. One of skill will appreciate that the hybrid approach can be used to identify a candidate therapeutic for any cancer having additional biomarkers that provide theranostic or prognostic information, including the cancers disclosed herein.

The genes and gene products used for molecular profiling, e.g., by IHC, ISH, sequencing (e.g., NGS), and/or PCR (e.g., qPCR), can be selected from those listed in any of Tables 4-12, e.g., any of Tables 5-10, or according to Table 5. Assessing one or more biomarkers disclosed herein can be used for characterizing any of the cancers disclosed herein. Characterizing includes the diagnosis of a disease or condition, the prognosis of a disease or condition, the determination of a disease stage or a condition stage, a drug efficacy, a physiological condition, organ distress or organ rejection, disease or condition progression, therapy-related association to a disease or condition, or a specific physiological or biological state.

A cancer in a subject can be characterized by obtaining a biological sample from a subject and analyzing one or more biomarkers from the sample. For example, characterizing a cancer for a subject or individual may include detecting a disease or condition (including pre-symptomatic early stage detecting), determining the prognosis, diagnosis, or theranosis of a disease or condition, or determining the stage or progression of a disease or condition. Characterizing a cancer can also include identifying appropriate treatments or treatment efficacy for specific diseases, conditions, disease stages and condition stages, predictions and likelihood analysis of disease progression, particularly disease recurrence, metastatic spread or disease relapse. Characterizing can also be identifying a distinct type or subtype of a cancer. The products and processes described herein allow assessment of a subject on an individual basis, which can provide benefits of more efficient and economical decisions in treatment.

In an aspect, characterizing a cancer includes predicting whether a subject is likely to respond to a treatment for the cancer. As used herein, a “responder” responds to or is predicted to respond to a treatment and a “non-responder” does not respond or is predicted to not respond to the treatment. Biomarkers can be analyzed in the subject and compared to biomarker profiles of previous subjects that were known to respond or not to a treatment. If the biomarker profile in a subject more closely aligns with that of previous subjects that were known to respond to the treatment, the subject can be characterized, or predicted, as a responder to the treatment. Similarly, if the biomarker profile in the subject more closely aligns with that of previous subjects that did not respond to the treatment, the subject can be characterized, or predicted as a non-responder to the treatment.

The sample used for characterizing a cancer can be any disclosed herein, including without limitation a tissue sample, tumor sample, or a bodily fluid. Bodily fluids that can be used included without limitation peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen (including prostatic fluid), Cowper's fluid or pre-ejaculatory fluid, female ejaculate, sweat, fecal matter, hair, tears, cyst fluid, pleural and peritoneal fluid, pericardial fluid, malignant effusion, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates or other lavage fluids. In an embodiment, the sample comprises vesicles. The biomarkers can be associated with the vesicles. In some embodiments, vesicles are isolated from the sample and the biomarkers associated with the vesicles are assessed.

Molecular profiling according to the invention can be used to guide treatment selection for cancers at any stage of disease or prior treatment. Molecular profiling comprises assessment of various biological characteristics including without limitation DNA mutations, gene rearrangements, gene copy number variation, RNA expression, gene fusions, protein expression, as well as assessment of other biological entities and phenomena that can inform clinical decision making. In some embodiments, the methods herein are used to guide selection of candidate treatments using the standard of care treatments for a particular type or lineage of cancer. Profiling of biomarkers that implicate standard-of-care treatments may be used to assist in treatment selection for a newly diagnosed cancer having multiple treatment options. Standard-of-care treatments may comprise NCCN on-compendium treatments or other standard treatments used for a cancer of a given lineage. One of skill will appreciate that such profiles can be updated as the standard of care and/or availability of experimental agents for a given disease lineage change. In other embodiments, molecular profiling is performed for additional biomarkers to identify treatments as beneficial or not beyond that go beyond the standard-of-care for a particular lineage or stage of the cancer. Such comprehensive profiling can be performed to assess a wide panel of druggable or drug-associated biomarker targets for any biological sample or specimen of interest. The comprehensive profile can also be used to guide selection of candidate treatments for any cancer at any point of care. The comprehensive profile may also be preferable when standard-of-care treatments not expected to provide further benefit, such as in the salvage treatment setting for recurrent cancer or wherein all standard treatments have been exhausted. For example, the comprehensive profile may be used to assist in treatment selection when standard therapies are not an option for any reason including, without limitation, when standard treatments have been exhausted for the patient. The comprehensive profile may be used to assist in treatment selection for highly aggressive or rare tumors with uncertain treatment regimens. For example, a comprehensive profile can be used to identify a candidate treatment for a newly diagnosed case or when the patient has exhausted standard of care therapies or has an aggressive disease. In practice, molecular profiling according to the invention has indeed identified beneficial therapies for a cancer patient when all standard-of-care treatments were exhausted the treating physician was unsure ofwhat treatment to select next. See the Examples herein. One of skill in the art will appreciate that by its very nature a comprehensive molecular profiling can be used to select a therapy for any appropriate indication independent of the nature of the indication (e.g., source, stage, prior treatment, etc). However, in some embodiments, a comprehensive molecular profile is tailored for a particular indication. For example, biomarkers associated with treatments that are known to be ineffective for a cancer from a particular lineage or anatomical origin may not be assessed as part of a comprehensive molecular profile for that particular cancer. Similarly, biomarkers associated with treatments that have been previously used and failed for a particular patient may not be assessed as part of a comprehensive molecular profile for that particular patient. In yet another non-limiting example, biomarkers associated with treatments that are only known to be effective for a cancer from a particular anatomical origin may only be assessed as part of a comprehensive molecular profile for that particular cancer. One of skill will further appreciate that the comprehensive molecular profile can be updated to reflect advancements, e.g., new treatments, new biomarker-drug associations, and the like, as available.

### Molecular Intelligence Profiles

The invention provides molecular intelligence (MI) molecular profiles using a variety of techniques to assess panels of biomarkers in order to identity candidate therapeutics as potentially beneficial or potentially of lack of benefit for treating a cancer. Such techniques comprise IHC for protein expression profiling, CISH/FISH for DNA copy number and rearrangement, and Sanger sequencing, pyrosequencing, PCR, RFLP, fragment analysis and Next Generation sequencing for aspects such as mutations (including insertions and deletions), fusions, copy number and expression. Exemplary profiles are described in Tables 5-10 herein. The profiling can be performed using the biomarker—drug associations and related rules for the various cancer lineages as described herein. In some embodiments, the associations are according to any one of Tables 2-3 or Table 11. Additional biomarker—drug associations can be found in any of International Patent Publications WO/2007/137187 (Int'l Appl. No. PCT/US2007/069286), published Nov. 29, 2007; WO/2010/045318 (Int'l Appl. No. PCT/US2009/060630), published Apr. 22, 2010; WO/2010/093465 (Int'l Appl. No. PCT/US2010/000407), published Aug. 19, 2010; WO/2012/170715 (Int'l Appl. No. PCT/US2012/041393), published Dec. 13, 2012; WO/2014/089241 (Int'l Appl. No. PCT/US2013/073184), published Jun. 12, 2014; WO/2011/056688 (Int'l Appl. No. PCT/US2010/054366), published May 12, 2011; WO/2012/092336 (Int'l Appl. No. PCT/US2011/067527), published Jul. 5, 2012; WO/2015/116868 (Int'l Appl. No. PCT/US2015/013618), published Aug. 6, 2015; WO/2017/053915 (Int'l Appl. No. PCT/US2016/053614), published Mar. 30, 2017; and WO/2016/141169 (Int'l Appl. No. PCT/US2016/020657), published Sep. 9, 2016; each of which publications is incorporated by reference herein in its entirety. Molecular intelligence profiles may include analysis of a panel of genes linked to known therapies and clinical trials, as well as genes that are known to be involved in cancer and have alternative clinical utilities including predictive, prognostic or diagnostic uses, genes provided in Tables 5-10 without a drug association denoted in Table 11. The panel may be assessed using Next Generation sequencing analysis, e.g., according to the panel of genes and characteristics in Tables 6-10.

The biomarkers which comprise the molecular intelligence molecular profiles can include genes or gene products that are known to be associated directly with a particular drug or class of drugs. The biomarkers can also be genes or gene products that interact with such drug associated targets, e.g., as members of a common pathway. The biomarkers can be selected from any of International Patent Publications WO/2007/137187 (Int'l Appl. No. PCT/US2007/069286), published Nov. 29, 2007; WO/2010/045318 (Int'l Appl. No. PCT/US2009/060630), published Apr. 22, 2010; WO/2010/093465 (Int'l Appl. No. PCT/US2010/000407), published Aug. 19, 2010; WO/2012/170715 (Int'l Appl. No. PCT/US2012/041393), published Dec. 13, 2012; WO/2014/089241 (Int'l Appl. No. PCT/US2013/073184), published Jun. 12, 2014; WO/2011/056688 (Int'l Appl. No. PCT/US2010/054366), published May 12, 2011; WO/2012/092336 (Int'l Appl. No. PCT/US2011/067527), published Jul. 5, 2012; WO/2015/116868 (Int'l Appl. No. PCT/US2015/013618), published Aug. 6, 2015; WO/2017/053915 (Int'l Appl. No. PCT/US2016/053614), published Mar. 30, 2017; and WO/2016/141169 (Int'l Appl. No. PCT/US2016/020657), published Sep. 9, 2016; each of which publications is incorporated by reference herein in its entirety. In some embodiments, the genes and/or gene products included in the molecular intelligence (MI) molecular profiles are selected from Table 4. For example, the molecular profiles can be performed for at least one, e.g., at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75 or 76 of 1p19q, ABL1, AKT1, ALK, APC, AR, AREG, ATM, BRAF, BRCA1, BRCA2, CDH1, CSF1R, CTNNB1, EGFR, EGFRvIII, ER, ERBB2, ERBB3, ERBB4, ERCC1, EREG, FBXW7, FGFR1, FGFR2, FLT3, GNAII, GNAQ, GNAS, H3K36me3, HNF1A, HRAS, IDH1, IDH2, JAK2, JAK3, KDR, KIT (cKit), KRAS, MET (cMET), MGMT, MLH1, MPL, MSH2, MSH6, MSI, NOTCHI, NPM1, NRAS, PBRM1, PDGFRA, PD-1, PD-L1, PGP, PIK3CA (PI3K), PMS2, PR, PTEN, PTPN11, RBi, RET, ROS1, RRM1, SMAD4, SMARCB1, SMO, SPARC, STK11, TLE3, TOP2A, TOPO1, TP53, TS, TUBB3, VHL, and VEGFR2. The biomarkers can be assessed using the laboratory methods as listed in Tables 5-11, or using similar analysis methodology such as disclosed herein.

TABLE 4
Exemplary Genes and Gene Products and Related Therapies

| | |
|---|---|
| 1p19q | 1p19q codeletions result from an unbalanced translocation between the p and q arms in |
| | chromosomes 1 and 19, respectively. Along with IDH mutations, 1p19q deletions are |
| | associated with oligodendroglioma tumorigenesis. Rates of 1p19q codeletion are |
| | especially high in low-grade and anaplastic oligodendroglioma. By contrast, 1p19q |
| | codeletions are lower in high grade gliomas like anaplastic astrocytoma and |
| | glioblastoma multiforme. NCCN Central Nervous System Guidelines mention 1p19q |
| | codeletions are indicative of a better prognosis in oligodendroglioma. Prospective |
| | studies indicate 1p19q codeletions are associated with potential benefit to PCV |
| | (procarbazine, CCNU [lomustine], vincristine) chemotherapy in anaplastic |
| | oligodendroglial tumors. |
| ABL1 | ABL1 also known as Abelson murine leukemia homolog 1. Most CML patients have a |
| | chromosomal abnormality due to a fusion between Abelson (Abl) tyrosine kinase gene |
| | at chromosome 9 and break point cluster (Bcr) gene at chromosome 22 resulting in |
| | constitutive activation of the Bcr-Abl fusion gene. Imatinib is a Bcr-Abl tyrosine kinase |
| | inhibitor commonly used in treating CML patients. Mutations in the ABL1 gene are |
| | common in imatinib resistant CML patients which occur in 30-90% of patients. |
| | However, more than 50 different point mutations in the ABL1 kinase domain may be |
| | inhibited by the second generation kinase inhibitors, dasatinib, bosutinib and nilotinib. |
| | The gatekeeper mutation, T315I that causes resistance to all currently approved TKIs |
| | accounts for about 15% of the mutations found in patients with imatinib resistance. |
| | BCR-ABL1 mutation analysis is recommended to help facilitate selection of |
| | appropriate therapy for patients with CML after treatment with imatinib fails. |
| AKT1 | AKT1 gene (v-akt murine thymoma viral oncogene homologue 1) encodes a |
| | serine/threonine kinase which is a pivotal mediator of the PI3K-related signaling |
| | pathway, affecting cell survival, proliferation and invasion. Dysregulated AKT activity |
| | is a frequent genetic defect implicated in tumorigenesis and has been indicated to be |
| | detrimental to hematopoiesis. Activating mutation E17K has been described in breast |
| | (2-4%), endometrial (2-4%), bladder cancers (3%), NSCLC (1%), squamous cell |
| | carcinoma of the lung (5%) and ovarian cancer (2%). This mutation in the pleckstrin |
| | homology domain facilitates the recruitment of AKT to the plasma membrane and |
| | subsequent activation by altering phosphoinositide binding. A mosaic activating |
| | mutation E17K has also been suggested to be the cause of Proteus syndrome. Mutation |
| | E49K has been found in bladder cancer, which enhances AKT activation and shows |
| | transforming activity in cell lines. |
| ALK | ALK or anaplastic lymphoma receptor tyrosine kinase belongs to the insulin receptor |
| | superfamily. It has been found to be rearranged or mutated in tumors including |
| | anaplastic large cell lymphomas, neuroblastoma, anaplastic thyroid cancer and non- |
| | small cell lung cancer. EML4-ALK fusion or point mutations of ALK result in the |
| | constitutively active ALK kinase, causing aberrant activation of downstream signaling |
| | pathways including RAS-ERK, JAK3-STAT3 and PI3K-AKT. Patients with an EML4- |
| | ALK rearrangement are likely to respond to the ALK-targeted agent crizotinib and |
| | ceritinib. ALK secondary mutations found in NSCLC have been associated with |
| | acquired resistance to ALK inhibitor, crizotinib and ceritinib. |
| AR | The androgen receptor (AR) gene encodes for the androgen receptor protein, a member |
| | of the steroid receptor family. Like other members of the nuclear steroid receptor |
| | family, AR is a DNA-binding transcription factor activated by specific hormones, in |
| | this case testosterone or DHT. Mutations of this gene are not often found in untreated, |
| | localized prostate cancer. Instead, they occur more frequently in hormone-refractory, |
| | androgen-ablated, and metastatic tumors. Recent findings indicate that specific |
| | mutations in AR (e.g. F876L, AR-V7) are associated with resistance to newer- |
| | generation, AR-targeted therapies such as enzalutamide. |
| APC | APC or adenomatous polyposis coli is a key tumor suppressor gene that encodes for a |
| | large multi-domain protein. This protein exerts its tumor suppressor function in the |
| | Wnt/β-catenin cascade mainly by controlling the degradation of β-catenin, the central |
| | activator of transcription in the Wnt signaling pathway. The Wnt signaling pathway |
| | mediates important cellular functions including intercellular adhesion, stabilization of |
| | the cytoskeleton, and cell cycle regulation and apoptosis, and it is important in |
| | embryonic development and oncogenesis. Mutation in APC results in a truncated |
| | protein product with abnormal function, lacking the domains involved in β-catenin |
| | degradation. Somatic mutation in the APC gene can be detected in the majority of |
| | colorectal tumors (80%) and it is an early event in colorectal tumorigenesis. APC wild |
| | type patients have shown better disease control rate in the metastatic setting when |
| | treated with oxaliplatin, while when treated with fluoropyrimidine regimens, APC wild |
| | type patients experience more hematological toxicities. APC mutation has also been |
| | identified in oral squamous cell carcinoma, gastric cancer as well as hepatoblastoma |
| | and may contribute to cancer formation. Germline mutation in APC causes familial |
| | adenomatous polyposis, which is an autosomal dominant inherited disease that will |
| | inevitably develop to colorectal cancer if left untreated. COX-2 inhibitors including |
| | celecoxib may reduce the recurrence of adenomas and incidence of advanced adenomas |
| | in individuals with an increased risk of CRC. Turcot syndrome and Gardner's syndrome |
| | have also been associated with germline APC defects. Germline mutations of the APC |
| | have also been associated with an increased risk of developing desmoid disease, |
| | papillary thyroid carcinoma and hepatoblastoma. |
| AREG | AREG, also known as amphiregulin, is a ligand of the epidermal growth factor |
| | receptor. Overexpression of AREG in primary colorectal cancer patients has been |
| | associated with increased clinical benefit from cetuximab in KRAS wildtype patients. |
| ATM | ATM or ataxia telangiectasia mutated is activated by DNA double-strand breaks and |
| | DNA replication stress. It encodes a protein kinase that acts as a tumor suppressor and |
| | regulates various biomarkers involved in DNA repair, which include p53, BRCA1, |
| | CHK2, RAD17, RAD9, and NBS1. Although ATM is associated with hematologic |
| | malignancies, somatic mutations have been found in colon (18%), head and neck |
| | (14%), and prostate (12%) cancers. Inactivating ATM mutations make patients |
| | potentially more susceptible to PARP inhibitors. Germline mutations in ATM are |
| | associated with ataxia-telangiectasia (also known as Louis-Bar syndrome) and a |
| | predisposition to malignancy. |
| BRAF | BRAF encodes a protein belonging to the raf/mil family of serine/threonine protein |
| | kinases. This protein plays a role in regulating the MAP kinase/ERK signaling pathway |
| | initiated by EGFR activation, which affects cell division, differentiation, and secretion. |
| | BRAF somatic mutations have been found in melanoma (43%), thyroid (39%), biliary |
| | tree (14%), colon (12%), and ovarian tumors (12%). A BRAF enzyme inhibitor, |
| | vemurafenib, was approved by FDA to treat unresectable or metastatic melanoma |
| | patients harboring BRAF V600E mutations. BRAF inherited mutations are associated |
| | with Noonan/Cardio-Facio-Cutaneous (CFC) syndrome, syndromes associated with |
| | short stature, distinct facial features, and potential heart/skeletal abnormalities. |
| BRCA1 | BRCA1 or breast cancer type 1 susceptibility gene encodes a protein involved in cell |
| | growth, cell division, and DNA-damage repair. It is a tumor suppressor gene which |
| | plays an important role in mediating double-strand DNA breaks by homologous |
| | recombination (HR). Tumors with BRCA1 mutation may be more sensitive to platinum |
| | agents and PARP inhibitors. |
| BRCA2 | BRCA2 or breast cancer type 2 susceptibility gene encodes a protein involved in cell |
| | growth, cell division, and DNA-damage repair. It is a tumor suppressor gene which |
| | plays an important role in mediating double-strand DNA breaks by homologous |
| | recombination (HR). Tumors with BRCA2 mutation may be more sensitive to platinum |
| | agents and PARP inhibitors. |
| CDH1 | This gene is a classical cadherin from the cadherin superfamily. The encoded protein is |
| | a calcium dependent cell-cell adhesion glycoprotein comprised of five extracellular |
| | cadherin repeats, a transmembrane region and a highly conserved cytoplasmic tail. The |
| | protein plays a major role in epithelial architecture, cell adhesion and cell invasion. |
| | Mutations in this gene are correlated with gastric, breast, colorectal, thyroid and |
| | ovarian cancer. Loss of function is thought to contribute to progression in cancer by |
| | increasing proliferation, invasion, and/or metastasis. The ectodomain of this protein |
| | mediates bacterial adhesion to mammalian cells and the cytoplasmic domain is required |
| | for internalization. |
| CSF1R | CSF1R or colony stimulating factor 1 receptor gene encodes a transmembrane tyrosine |
| | kinase, a member of the CSF1/PDGF receptor family. CSF1R mediates the cytokine |
| | (CSF-1) responsible for macrophage production, differentiation, and function. |
| | Although associated with hematologic malignancies, mutations of this gene are |
| | associated with cancers of the liver (21%), colon (13%), prostate (3%), endometrium |
| | (2%), and ovary (2%). It is suggested that patients with CSF1R mutations could |
| | respond to imatinib. Germline mutations in CSF1R are associated with diffuse |
| | leukoencephalopathy, a rapidly progressive neurodegenerative disorder. |
| CTNNB1 | CTNNB1 or cadherin-associated protein, beta 1, encodes for β-catenin, a central |
| | mediator of the Wnt signaling pathway which regulates cell growth, migration, |
| | differentiation and apoptosis. Mutations in CTNNB1 (often occurring in exon 3) |
| | prevent the breakdown of β-catenin, which allows the protein to accumulate resulting |
| | in persistent transactivation of target genes, including c-myc and cyclin-D1. Somatic |
| | CTNNB1 mutations occur in 1-4% of colorectal cancers, 2-3% of melanomas, 25-38% |
| | of endometrioid ovarian cancers, 84-87% of sporadic desmoid tumors, as well as the |
| | pediatric cancers, hepatoblastoma, medulloblastoma and Wilms' tumors. |
| EGFR | EGFR or epidermal growth factor receptor, is a transmembrane receptor tyrosine kinase |
| | belonging to the ErbB family of receptors. Upon ligand binding, the activated receptor |
| | triggers a series of intracellular pathways (Ras/MAPK, PI3K/Akt, JAK-STAT) that |
| | result in cell proliferation, migration and adhesion. EGFR mutations have been |
| | observed in 20-25% of non-small cell lung cancer (NSCLC), 10% of endometrial and |
| | peritoneal cancers. Somatic gain-of-function EGFR mutations, including in-frame |
| | deletions in exon 19 or point mutations in exon 21, confer sensitivity to first- and |
| | second-generation tyrosine kinase inhibitors (TKIs, e.g., erlotinib, gefitinib and |
| | afatinib), whereas the secondary mutation, T790M in exon 20, confers reduced |
| | response. Non-small cell lung cancer cancer patients overexpressing EGFR protein |
| | have been found to respond to the EGFR monoclonal antibody, cetuximab. Germline |
| | mutations and polymorphisms of EGFR have been associated with familial lung |
| | adenocarcinomas. |
| EGFRvIII | EGFRvIII is a mutated form of EGFR with deletion of exon 2 to 7 on the extracellular |
| | ligand-binding domain. This genetic alteration has been found in about 30% of |
| | glioblastoma, 30% of head and neck squamous cell cancer, 30% of breast cancer and |
| | 15% of NSCLC, and has not been found in normal tissue. EGFRvIII can form homo- |
| | dimers or heterodimers with EGFR or ERBB2, resulting in constitutive activation in |
| | the absence of ligand binding, activating various downstream signaling pathways |
| | including the PI3K and MAPK pathways, leading to increased cell proliferation and |
| | motility as well as inhibition of apoptosis. Preliminary studies have shown that |
| | EGFRvIII expression may associate with higher sensitivity to erlotinib and gefitinib, as |
| | well as to pan-Her inhibitors including neratinib and dacomitinib. EGFRvIII peptide |
| | vaccine rindopepimut (CDX-110) and monoclonal antibodies specific to EGFRvIII |
| | including ABT-806 and AMG595 are being investigated in clinical trials. |
| ER | The estrogen receptor (ER) is a member of the nuclear hormone family of intracellular |
| | receptors which is activated by the hormone estrogen. It functions as a DNA binding |
| | transcription factor to regulate estrogen-mediated gene expression. Estrogen receptors |
| | overexpressing breast cancers are referred to as ‘ER positive.’ Estrogen binding to ER |
| | on cancer cells leads to cancer cell proliferation. Breast tumors over-expressing ER are |
| | treated with hormone-based anti-estrogen therapy. For example, everolimus combined |
| | with exemestane may improve survival in ER positive Her2 negative breast cancer |
| | patients who are resistant to aromatase inhibitors. |
| ERBB2 | ERBB2 (HER2 (human epidermal growth factor receptor 2)) or v-erb-b2 erythroblastic |
| | leukemia viral oncogene homolog 2, encodes a member of the epidermal growth factor |
| | (EGF) receptor family of receptor tyrosine kinases. This gene binds to other ligand- |
| | bound EGF receptor family members to form a heterodimer and enhances kinase- |
| | mediated activation of downstream signaling pathways, leading to cell proliferation. |
| | Most common mechanism for activation of HER2 are gene amplification and over- |
| | expression with somatic mutations being rare. Her2 is overexpressed in 15-30% of |
| | newly diagnosed breast cancers. Clinically, Her2 is a target for the monoclonal |
| | antibodies trastuzumab and pertuzumab which bind to the receptor extracellularly; the |
| | kinase inhibitor lapatinib binds and blocks the receptor intracellularly. |
| ERBB3 | ERBB3 encodes a protein (HER3 (human epidermal growth factor receptor 3)) that is a |
| | member of the EGFR family of protein tyrosine kinases. ERBB3 protein does not |
| | actually contain a kinase domain itself, but it can activate other members of the EGFR |
| | kinase family by forming heterodimers. Heterodimerization with other kinases triggers |
| | an intracellular cascade increasing cell proliferation. Mutations in ERBB3 have been |
| | observed primarily in gastric cancer and cancer of the gall bladder. Other tissue types |
| | known to harbor ERBB3 mutations include hormone-positive breast cancer, |
| | glioblastoma, ovarian, colon, head and neck and lung. |
| ERBB4 | ERBB4 (HER4) is a member of the Erbb receptor family known to play a pivotal role |
| | in cell-cell signaling and signal transduction regulating cell growth and development. |
| | The most commonly affected signaling pathways are the PI3K-Akt and MAP kinase |
| | pathways. Erbb4 was found to be somatically mutated in 19% of melanomas and Erbb4 |
| | mutations may confer “oncogene addiction” on melanoma cells. Erbb4 mutations have |
| | also been observed in various other cancer types, including, gastric carcinomas (2%), |
| | colorectal carcinomas (1-3%), non-small cell lung cancer (2-5%) and breast carcinomas |
| | (1%). |
| ERCC1 | ERCC1, or excision repair cross-complementation group 1, is a key component of the |
| | nucleotide excision repair (NER) pathway. NER is a DNA repair mechanism necessary |
| | for the repair of DNA damage from a variety of sources including platinum agents. |
| | Tumors with low expression of ERCC1 have impaired NER capacity and may be more |
| | sensitive to platinum agents. |
| EREG | EREG, also known as epiregulin, is a ligand of the epidermal growth factor receptor. |
| | Overexpression of EREG in primary colorectal cancer patients has been related to |
| | clinical outcome in KRAS wildtype patients treated with cetuximab indicating ligand |
| | driven autocrine oncogenic EGFR signaling. |
| FBXW7 | FBXW7 or E3 ligase F-box and WD repeat domain containing 7, also known as Cdc4, |
| | encodes three protein isoforms which constitute a component of the ubiquitin- |
| | proteasome complex. Mutation of FBXW7 occurs in hotspots and disrupts the |
| | recognition of and binding with substrates which inhibits the proper targeting of |
| | proteins for degradation (e.g. Cyclin E, c-Myc, SREBP1, c-Jun, Notch-1, mTOR and |
| | MCL1). Mutation frequencies identified in cholangiocarcinomas, acute T- |
| | lymphoblastic leukemia/lymphoma, and carcinomas of endometrium, colon and |
| | stomach are 35%, 31%, 9%, 9%, and 6%, respectively. Targeting an oncoprotein |
| | downstream of FBXW7, such as mTOR or c-Myc, may provide a therapeutic strategy. |
| | Tumor cells with mutated FBXW7 may be sensitive to rapamycin treatment, |
| | suggesting FBXW7 loss (mutation) may be a predictive biomarker for treatment with |
| | inhibitors of the mTOR pathway. In addition, it has been proposed that loss of FBXW7 |
| | confers resistance to tubulin-targeting agents like paclitaxel or vinorelbine, by |
| | interfering with the degradation of MCL1, a regulator of apoptosis. |
| FGFR1 | FGFR1 or fibroblast growth factor receptor 1, encodes for FGFR1 which is important |
| | for cell division, regulation of cell maturation, formation of blood vessels, wound |
| | healing and embryonic development. Somatic activating mutations are rare, but have |
| | been documented in melanoma, glioblastoma, and lung tumors. Germline, gain-of- |
| | function mutations in FGFR1 result in developmental disorders including Kallmann |
| | syndrome and Pfeiffer syndrome. Preclinical studies suggest that FGFR1 amplification |
| | may be associated with endocrine resistance in breast cancer. FGFR1 amplification has |
| | been observed in various cancer types including breast cancer, squamous cell lung |
| | cancer, head and neck squamous cell cancer and esophageal cancer and may indicate |
| | sensitivity to FGFR-targeted therapies. |
| FGFR2 | FGFR2 is a receptor for fibroblast growth factor. Activation of FGFR2 through |
| | mutation and amplification has been noted in a number of cancers. Somatic mutations |
| | of the fibroblast growth factor receptor 2 (FGFR2) tyrosine kinase are present in |
| | endometrial carcinoma, lung squamous cell carcinoma, cervical carcinoma, and |
| | melanoma. In the endometrioid histology of endometrial cancer, the frequency of |
| | FGFR2 mutation is 16% and the mutation is associated with shorter disease free |
| | survival in patients diagnosed with early stage disease. Loss of function FGFR2 |
| | mutations occur in about 8% melanomas and contribute to melanoma pathogenesis. |
| | Germline mutations in FGFR2 are associated with numerous medical conditions that |
| | include congenital craniofacial malformation disorders, Apert syndrome and the related |
| | Pfeiffer and Crouzon syndromes. Amplification of FGFR2 has been shown in 5-10% of |
| | gastric cancer and breast cancer and may indicate sensitivity to FGFR-targeted |
| | therapies. |
| FLT3 | FLT3 or Fms-like tyrosine kinase 3 receptor is a member of class III receptor tyrosine |
| | kinase family, which includes PDGFRA/B and KIT. Signaling through FLT3 ligand- |
| | receptor complex regulates hematopoiesis, specifically lymphocyte development. The |
| | FLT3 internal tandem duplication (FLT3-ITD) is the most common genetic lesion in |
| | acute myeloid leukemia (AML), occurring in 25% of cases. FLT3 mutations are |
| | uncommon in solid tumors; however they have been documented in breast cancer. |
| GNA11 | GNA11 is a proto-oncogene that belongs to the Gq family of the G alpha family of G |
| | protein coupled receptors. Known downstream signaling partners of GNA11 are |
| | phospholipase C beta and RhoA and activation of GNA11 induces MAPK activity. |
| | Over half of uveal melanoma patients lacking a mutation in GNAQ exhibit somatic |
| | mutations in GNA11. Activating mutations of GNA11 have not been found in other |
| | malignancies. |
| GNAQ | This gene encodes the Gq alpha subunit of G proteins. G proteins are a family of |
| | heterotrimeric proteins coupling seven-transmembrane domain receptors. Oncogenic |
| | mutations in GNAQ result in a loss of intrinsic GTPase activity, resulting in a |
| | constitutively active Galpha subunit. This results in increased signaling through the |
| | MAPK pathway. Somatic mutations in GNAQ have been found in 50% of primary |
| | uveal melanoma patients and up to 28% of uveal melanoma metastases. |
| GNAS | GNAS (or GNAS complex locus) encodes a stimulatory G protein alpha-subunit. These |
| | guanine nucleotide binding proteins (G proteins) are a family of heterotrimeric proteins |
| | which couple seven-transmembrane domain receptors to intracellular cascades. |
| | Stimulatory G-protein alpha-subunit transmits hormonal and growth factor signals to |
| | effector proteins and is involved in the activation of adenylate cyclases. Mutations of |
| | GNAS gene at codons 201 or 227 lead to constitutive cAMP signaling. GNAS somatic |
| | mutations have been found in pituitary (28%), pancreatic (20%), ovarian (11%), |
| | adrenal gland (6%), and colon (6%) cancers. Patients with somatic GNAS mutations |
| | may derive benefit from clinical trials with MEK inhibitors. Germline mutations of |
| | GNAS have been shown to be the cause of McCune-Albright syndrome (MAS), a |
| | disorder marked by endocrine, dermatologic, and bone abnormalities. GNAS is usually |
| | found as a mosaic mutation in patients. Loss of function mutations are associated with |
| | pseudohypoparathyroidism and pseudopseudohypoparathyroidism. |
| H3K36me3 | Trimethylated histone H3 lysine 36 (H3K36me3) is a chromatin regulatory protein that |
| | regulates gene expression. A loss of H3K36me3 protein correlates with loss of |
| | expression or mutation of SETD2 which is a member of the SET domain family of |
| | histone methyltransferases. Loss of SETD2 as well as H3K36m3 protein has been |
| | detected in various solid tumors including renal cell carcinoma and breast cancer and |
| | leads to poor prognosis. |
| HRAS | HRAS (homologous to the oncogene of the Harvey rat sarcoma virus), together with |
| | KRAS and NRAS, belong to the superfamily of RAS GTPase. RAS protein activates |
| | RAS-MEK-ERK/MAPK kinase cascade and controls intracellular signaling pathways |
| | involved in fundamental cellular processes such as proliferation, differentiation, and |
| | apoptosis. Mutant Ras proteins are persistently GTP-bound and active, causing severe |
| | dysregulation of the effector signaling. HRAS mutations have been identified in |
| | cancers from the urinary tract (10%-40%), skin (6%) and thyroid (4%) and they |
| | account for 3% of all RAS mutations identified in cancer. RAS mutations (especially |
| | HRAS mutations) occur (5%) in cutaneous squamous cell carcinomas and |
| | keratoacanthomas that develop in patients treated with BRAF inhibitor vemurafenib, |
| | likely due to the paradoxical activation of the MAPK pathway. Germline mutation in |
| | HRAS has been associated with Costello syndrome, a genetic disorder that is |
| | characterized by delayed development and mental retardation and distinctive facial |
| | features and heart abnormalities. |
| IDH1 | IDH1 encodes for isocitrate dehydrogenase in cytoplasm and is found to be mutated in |
| | 60-90% of secondary gliomas, 75% of cartilaginous tumors, 17% of thyroid tumors, |
| | 15% of cholangiocarcinoma, 12-18% of patients with acute myeloid leukemia, 5% of |
| | primary gliomas, 3% of prostate cancer, as well as in less than 2% in paragangliomas, |
| | colorectal cancer and melanoma. Mutated IDH1 results in impaired catalytic function |
| | of the enzyme, thus altering normal physiology of cellular respiration and metabolism. |
| IDH2 | IDH2 encodes for the mitochondrial form of isocitrate dehydrogenase, a key enzyme in |
| | the citric acid cycle, which is essential for cell respiration. Mutation in IDH2 not only |
| | results in impaired catalytic function of the enzyme, but also causes the overproduction |
| | of an onco-metabolite, 2-hydroxy-glutarate, which can extensively alter the |
| | methylation profile in cancer. IDH2 mutation is mutually exclusive of IDH1 mutation, |
| | and has been found in 2% of gliomas and 10% of AML, as well as in cartilaginous |
| | tumors and cholangiocarcinoma. In gliomas, IDH2 mutations are associated with lower |
| | grade astrocytomas, oligodendrogliomas (grade II/III), as well as secondary |
| | glioblastoma (transformed from a lower grade glioma), and are associated with a better |
| | prognosis. In secondary glioblastoma, preliminary evidence suggests that IDH2 |
| | mutation may associate with a better response to alkylating agent temozolomide. IDH |
| | mutations have also been suggested to associate with a benefit from using |
| | hypomethylating agents in cancers including AML. Germline IDH2 mutation has been |
| | indicated to associate with a rare inherited neurometabolic disorder D-2- |
| | hydroxyglutaric aciduria. |
| JAK2 | JAK2 or Janus kinase 2 is a part of the JAK/STAT pathway which mediates multiple |
| | cellular responses to cytokines and growth factors including proliferation and cell |
| | survival. It is also essential for numerous developmental and homeostatic processes, |
| | including hematopoiesis and immune cell development. Mutations in the JAK2 kinase |
| | domain result in constitutive activation of the kinase and the development of chronic |
| | myeloproliferative neoplasms such as polycythemia vera (95%), essential |
| | thrombocythemia (50%) and myelofibrosis (50%). JAK2 mutations were also found in |
| | BCR-ABL1-negative acute lymphoblastic leukemia patients and the mutated patients |
| | show a poor outcome. Germline mutations in JAK2 have been associated with |
| | myeloproliferative neoplasms and thrombocythemia. |
| JAK3 | JAK3 or Janus activated kinase 3 is an intracellular tyrosine kinase involved in |
| | cytokine signaling, while interacting with members of the STAT family. Like JAK1, |
| | JAK2, and TYK2, JAK3 is a member of the JAK family of kinases. When activated, |
| | kinase enzymes phosphorylate one or more signal transducer and activator of |
| | transcription (STAT) factors, which translocate to the cell nucleus and regulate the |
| | expression of genes associated with survival and proliferation. JAK3 signaling is |
| | related to T cell development and proliferation. This biomarker is found in |
| | malignancies including without limitation head and neck (21%) colon (7%), prostate |
| | (5%), ovary (4%), breast (2%), lung (1%), and stomach (1%) cancer. Its prognostic and |
| | predictive utility is under investigation. Germline mutations of JAK3 are associated |
| | with severe, combined immunodeficiency disease (SCID). |
| KDR | KDR (kinase insert domain receptor), also known as VEGFR2 (vascular endothelial |
| | growth factor 2), is one of three main subtypes of VEGFR and is expressed on almost |
| | all endothelial cells. This protein is an important signaling protein in angiogenesis. |
| | VEGFR2 copy number changes are frequently observed in lung, glioma and triple |
| | negative breast cancer. Evidence suggests that increased levels of VEGFR2 may be |
| | predictive of response to anti-angiogenic drugs and multi-targeted kinase inhibitors. |
| | Several VEGFR antagonists are either FDA-approved or in clinical trials (i.e. |
| | bevacizumab, cabozantinib, regorafenib, pazopanib, and vandetanib). |
| KIT (cKit) | c-KIT is a receptor tyrosine kinase expressed by hematopoietic stem cells, interstitial |
| | cells of cajal (pacemaker cells of the gut) and other cell types. Upon binding of c-KIT |
| | to stem cell factor (SCF), receptor dimerization initiates a phosphorylation cascade |
| | resulting in proliferation, apoptosis, chemotaxis and adhesion. C-KIT mutation has |
| | been identified in various cancer types including gastrointestinal stromal tumors |
| | (GIST) (up to 85%) and melanoma (chronic sun damage type, acral or mucosal) (20- |
| | 40%). C-KIT is inhibited by multi-targeted agents including imatinib and sunitinib. |
| KRAS | KRAS or V-Ki-ras2 Kirsten rat sarcoma viral oncogene homolog encodes a signaling |
| | intermediate involved in many signaling cascades including the EGFR pathway. KRAS |
| | somatic mutations have been found in pancreatic (57%), colon (35%), lung (16%), |
| | biliary tract (28%), and endometrial (15%) cancers. Mutations at activating hotspots are |
| | associated with resistance to EGFR tyrosine kinase inhibitors (erlotinib, gefitinib) in |
| | NSCLC and monoclonal antibodies (cetuximab, panitumumab) in CRC patients. |
| | Patients with KRAS G13D mutation have been shown to derive benefit from anti- |
| | EGFR monoclonal antibody therapy in CRC patients. Several germline mutations of |
| | KRAS (V14I, T58I, and D153V amino acid substitutions) are associated with Noonan |
| | syndrome. |
| MET (cMET) | MET is a proto-oncogene that encodes the tyrosine kinase receptor, cMET, of |
| | hepatocyte growth factor (HGF) or scatter factor (SF). cMet mutations cause aberrant |
| | MET signaling in various cancer types including renal papillary, hepatocellular, head |
| | and neck squamous, gastric carcinomas and non-small cell lung cancer. Specifically, |
| | mutations in the juxtamembrane domain (exon 14, 15) results in the constitutive |
| | activation and show enhanced tumorigenicity. Germline mutations in cMET have been |
| | associated with hereditary papillary renal cell carcinoma. |
| MGMT | O-6-methylguanine-DNA methyltransferase (MGMT) encodes a DNA repair enzyme. |
| | MGMT expression is mainly regulated at the epigenetic level through CpG island |
| | promoter methylation which in turn causes functional silencing of the gene. MGMT |
| | methylation and/or low expression has been correlated with response to alkylating |
| | agents like temozolomide and dacarbazine. |
| MLH1 | MLH1 or mutL homolog 1, colon cancer, nonpolyposis type 2 (E. coli) gene encodes a |
| | mismatch repair (MMR) protein which repairs DNA mismatches that occur during |
| | replication. Although the frequency is higher in colon cancer (10%), MLH1 somatic |
| | mutations have been found in esophageal (6%), ovarian (5%), urinary tract (5%), |
| | pancreatic (5%), and prostate (5%) cancers. Germline mutations of MLH1 are |
| | associated with Lynch syndrome, also known as hereditary non-polyposis colorectal |
| | cancer (HNPCC). Patients with Lynch syndrome are at increased risk for various |
| | malignancies, including intestinal, gynecologic, and upper urinary tract cancers and in |
| | its variant, Muir-Torre syndrome, with sebaceous tumors. |
| MPL | MPL or myeloproliferative leukemia gene encodes the thrombopoietin receptor, which |
| | is the main humoral regulator of thrombopoiesis in humans. MPL mutations cause |
| | constitutive activation of JAK-STAT signaling and have been detected in 5-7% of |
| | patients with primary myelofibrosis (PMF) and 1% of those with essential |
| | thrombocythemia (ET). |
| MSH2 | This locus is frequently mutated in hereditary nonpolyposis colon cancer (HNPCC). |
| | When cloned, it was discovered to be a human homolog of the E. coli mismatch repair |
| | gene mutS, consistent with the characteristic alterations in microsatellite sequences |
| | found in HNPCC. The protein product is a component of the DNA mismatch repair |
| | system (MMR), and forms two different heterodimers: MutS alpha (MSH2-MSH6 |
| | heterodimer) and MutS beta (MSH2-MSH3 heterodimer) which binds to DNA |
| | mismatches thereby initiating DNA repair. After mismatch binding, MutS alpha or beta |
| | forms a ternary complex with the MutL alpha heterodimer, which is thought to be |
| | responsible for directing the downstream MMR events. MutS alpha may also play a |
| | role in DNA homologous recombination repair. |
| MSH6 | This gene encodes a member of the DNA mismatch repair MutS family. Mutations in |
| | this gene may be associated with hereditary nonpolyposis colon cancer, colorectal |
| | cancer, and endometrial cancer. The protein product is a component of the DNA |
| | mismatch repair system (MMR), and heterodimerizes with MSH2 to form MutS alpha, |
| | which binds to DNA mismatches thereby initiating DNA repair. MutS alpha may also |
| | play a role in DNA homologous recombination repair. Recruited on chromatin in G1 |
| | and early S phase via its PWWP domain that specifically binds trimethylated ‘Lys-36’ |
| | of histone H3 (H3K36me3): early recruitment to chromatin to be replicated allowing a |
| | TIL is associated with a poor prognosis in various cancer types including lymphoma |
| | and breast cancer. |
| PD-L1 | PD-L1 (programmed cell death ligand 1; also known as cluster of differentiation 274 |
| | (CD274) or B7 homolog 1 (B7-H1)) is a glycoprotein expressed in various tumor types |
| | and is associated with poor outcome. Upon binding to its receptor, PD-1, the PD-1/PD- |
| | L1 interaction functions to negatively regulate the immune system, attenuating |
| | antitumor immunity by maintaining an immunosuppressive tumor microenvironment. |
| | PD-L1 expression is upregulated in tumor cells through activation of common |
| | oncogenic pathways or exposure to inflammatory cytokines. Assessment of PD-L1 |
| | offers information on patient prognosis and also represents a target for immune |
| | manipulation in treatment of solid tumors. Clinical trials are currently recruiting |
| | patients with various tumor types testing immunomodulatory agents. |
| PDGFRA | PDGFRA is the alpha-type platelet-derived growth factor receptor, a surface tyrosine |
| | kinase receptor structurally homologous to c-KIT, which activates PIK3CA/AKT, |
| | RAS/MAPK and JAK/STAT signaling pathways. PDGFRA mutations are found in 5- |
| | 8% of patients with gastrointestinal stromal tumors (GIST) and increases to 30% in |
| | KIT wildtype GIST. Germline mutations in PDGFRA have been associated with |
| | Familial gastrointestinal stromal tumors and Hypereosinophillic Syndrome (HES). |
| PGP | P-glycoprotein (MDR1, ABCB1) is an ATP-dependent, transmembrane drug efflux |
| | pump with broad substrate specificity, which pumps antitumor drugs out of cells. Its |
| | expression is often induced by chemotherapy drugs and is thought to be a major |
| | mechanism of chemotherapy resistance. Overexpression of p-gp is associated with |
| | resistance to anthracylines (doxorubicin, epirubicin). P-gp remains the most important |
| | and dominant representative of Multi-Drug Resistance phenotype and is correlated with |
| | disease state and resistant phenotype. |
| PIK3CA (PI3K) | PIK3CA (phosphoinositide-3-kinase catalytic alpha polypeptide) encodes a protein in |
| | the PI3 kinase pathway. This pathway is an active target for drug development. |
| | PIK3CA somatic mutations have been found in breast (26%), endometrial (23%), |
| | urinary tract (19%), colon (13%), and ovarian (11%) cancers. PIK3CA exon 20 |
| | mutations have been associated with benefit from mTOR inhibitors (everolimus, |
| | temsirolimus). Evidence suggests that breast cancer patients with activation of the |
| | PI3K pathway due to PTEN loss or PIK3CA mutation/amplification have a |
| | significantly shorter survival following trastuzumab treatment. PIK3CA mutated |
| | colorectal cancer patients are less likely to respond to EGFR targeted monoclonal |
| | antibody therapy. Somatic mosaic activating mutations in PIK3CA are said to cause |
| | CLOVES syndrome. |
| PMS2 | This gene encodes the postmeiotic segregation increased 2 (PMS2) protein involved in |
| | DNA mismatch repair. PMS2 forms a heterodimer with MLH1 and, together, this |
| | complex interacts with other complexes bound to mismatched bases. Loss of PMS2 |
| | leads to mismatch repair deficiency and microsatellite instability. Inactivating |
| | mutations in this gene are associated with protein loss and hereditary Lynch syndrome, |
| | the latter being linked with a lifetime risk for various malignancies, especially |
| | colorectal and endometrial cancer. |
| PR | The progesterone receptor (PR or PGR) is an intracellular steroid receptor that |
| | specifically binds progesterone, an important hormone that fuels breast cancer growth. |
| | PR positivity in a tumor indicates that the tumor is more likely to be responsive to |
| | hormone therapy by anti-estrogens, aromatase inhibitors and progestogens. |
| PTEN | PTEN or phosphatase and tensin homolog is a tumor suppressor gene that prevents |
| | cells from proliferating. PTEN is an important mediator in signaling downstream of |
| | EGFR, and loss of PTEN gene function/expression due to gene mutations or allele loss |
| | is associated with reduced benefit to EGFR-targeted monoclonal antibodies. Mutation |
| | in PTEN is found in 5-14% of colorectal cancer and 7% of breast cancer. PTEN |
| | mutation leads to loss of function of the encoded phosphatase, and an upregulation of |
| | the PIK3CA/AKT pathway. Germline PTEN mutations associate with Cowden disease |
| | and Bannayan-Riley-Ruvalcaba syndrome. These dominantly inherited disorders |
| | belong to a family of hamartomatous polyposis syndromes which feature multiple |
| | tumor-like growths (hamartomas) accompanied by an increased risk of breast |
| | carcinoma, follicular carcinoma of the thyroid, glioma, prostate and endometrial |
| | cancer. Trichilemmoma, a benign, multifocal neoplasm of the skin is also associated |
| | with PTEN germline mutations. |
| PTPN11 | PTPN11 or tyrosine-protein phosphatase non-receptor type 11 is a proto-oncogene that |
| | encodes a signaling molecule, Shp-2, which regulates various cell functions like |
| | mitogenic activation and transcription regulation. PTPN11 gain-of-function somatic |
| | mutations have been found to induce hyperactivation of the Akt and MAPK networks. |
| | Because of this hyperactivation, Ras effectors, such as Mek and PI3K, are potential |
| | targets for novel therapeutics in those with PTPN11 gain-of-function mutations. |
| | PTPN11 somatic mutations are found in hematologic and lymphoid malignancies (8%), |
| | gastric (2%), colon (2%), ovarian (2%), and soft tissue (2%) cancers. Germline |
| | mutations of PTPN11 are associated with Noonan syndrome, which itself is associated |
| | with juvenile myelomonocytic leukemia (JMML). PTPN11 is also associated with |
| | LEOPARD syndrome, which is associated with neuroblastoma and myeloid leukemia. |
| RB1 | RB1 or retinoblastoma-1 is a tumor suppressor gene whose protein regulates the cell |
| | cycle by interacting with various transcription factors, including the E2F family (which |
| | controls the expression of genes involved in the transition of cell cycle checkpoints). |
| | Besides ocular cancer, RB1 mutations have also been detected in other malignancies, |
| | such as ovarian (10%), bladder (41%), prostate (8%), breast (6%), brain (6%), colon |
| | (5%), and renal (2%) cancers. RB1 status, along with other mitotic checkpoints, has |
| | been associated with the prognosis of GIST patients. Germline mutations of RB1 are |
| | associated with the pediatric tumor, retinoblastoma. Inherited retinoblastoma is usually |
| | bilateral. Studies indicate patients with a history of retinoblastoma are at increased risk |
| | for secondary malignancies. |
| RET | RET or rearranged during transfection gene, located on chromosome 10, activates cell |
| | signaling pathways involved in proliferation and cell survival. RET mutations are |
| | found in 23-69% of sporadic medullary thyroid cancers (MTC), but RET fusions are |
| | common in papillary thyroid cancer, and more recently have been found in 1-2% of |
| | lung adenocarcinoma. Germline activating mutations of RET are associated with |
| | multiple endocrine neoplasia type 2 (MEN2), which is characterized by the presence of |
| | medullary thyroid carcinoma, bilateral pheochromocytoma, and primary |
| | hyperparathyroidism. Germline inactivating mutations of RET are associated with |
| | Hirschsprung's disease. |
| ROS1 | The proto-oncogene ROS1 is a receptor tyrosine kinase of the insulin receptor family. |
| | The ligand and function of ROS1 are unknown. Dimerization of ROS1-fused proteins |
| | results in constitutive activation of the receptor kinase, leading to cell proliferation and |
| | survival. Clinical data show that ROS-rearranged NSCLC patients have increased |
| | sensitivity and improved response to the MET/ALK/ROS inhibitor, crizotinib. |
| RRM1 | Ribonucleotide reductase subunit M1 (RRM1) is a component of the ribonucleotide |
| | reductase holoenzyme consisting of M1 and M2 subunits. The ribonucleotide reductase |
| | is a rate-limiting enzyme involved in the production of nucleotides required for DNA |
| | synthesis. Gemcitabine is a deoxycitidine analogue which inhibits ribonucleotide |
| | reductase activity. High RRM1 level is associated with resistance to gemcitabine. |
| SMAD4 | SMAD4 or mothers against decapentaplegic homolog 4, is one of eight proteins in the |
| | SMAD family, involved in multiple signaling pathways and are key modulators of the |
| | transcriptional responses to the transforming growth factor-β (TGFB) receptor kinase |
| | complex. SMAD4 resides on chromosome 18q21, one of the most frequently deleted |
| | chromosomal regions in colorectal cancer. Smad4 stabilizes Smad DNA-binding |
| | complexes and also recruits transcriptional coactivators such as histone |
| | acetyltransferases to regulatory elements. Dysregulation of SMAD4 occurs late in |
| | tumor development, and occurs through mutations of the MH1 domain which inhibits |
| | the DNA-binding function, thus dysregulating TGFBR signaling. Mutated (inactivated) |
| | SMAD4 is found in 50% of pancreatic cancers and 10-35% of colorectal cancers. |
| | Germline mutations in SMAD4 are associated with juvenile polyposis (JP) and |
| | combined syndrome of JP and hereditary hemorrhagic teleangiectasia (JP-HHT). |
| SMARCB1 | SMARCB1 also known as SWI/SNF related, matrix associated, actin dependent |
| | regulator of chromatin, subfamily b, member 1, is a tumor suppressor gene implicated |
| | in cell growth and development. Loss of expression of SMARCB1 has been observed |
| | in tumors including epithelioid sarcoma, renal medullary carcinoma, undifferentiated |
| | pediatric sarcomas, and a subset of hepatoblastomas. Germline mutation in SMARCB1 |
| | causes about 20% of all rhabdoid tumors which makes it important for clinicians to |
| | facilitate genetic testing and refer families for genetic counseling. Germline |
| | SMARCB1 mutations have also been identified as the pathogenic cause of a subset of |
| | schwannomas and meningiomas. |
| SMO | SMO (smoothened) is a G protein-coupled receptor which plays an important role in |
| | the Hedgehog signaling pathway. It is a key regulator of cell growth and differentiation |
| | during development, and is important in epithelial and mesenchymal interaction in |
| | many tissues during embryogenesis. Dysregulation of the Hedgehog pathway is found |
| | in cancers including basal cell carcinomas (12%) and medulloblastoma (1%). A gain- |
| | of-function mutation in SMO results in constitutive activation of hedgehog pathway |
| | signaling, contributing to the genesis of basal cell carcinoma. SMO mutations have |
| | been associated with the resistance to SMO antagonist GDC-0449 in medulloblastoma |
| | patients by blocking the binding to SMO. SMO mutation may also contribute partially |
| | to resistance to SMO antagonist LDE225 in BCC. Various clinical trials (on |
| | www.clinicaltrials.gov) investigating SMO antagonists may be available for SMO |
| | mutated patients. |
| SPARC | SPARC (secreted protein acidic and rich in cysteine) is a calcium-binding matricellular |
| | glycoprotein secreted by many types of cells. Studies indicate SPARC over-expression |
| | improves the response to the anticancer drug, nab-paclitaxel. The improved response is |
| | thought to be related to SPARC's role in accumulating albumin and albumin-targeted |
| | agents within tumor tissue. |
| STK11 | STK11 also known as LKB1, is a serine/threonine kinase. It is thought to be a tumor |
| | suppressor gene which acts by interacting with p53 and CDC42. It modulates the |
| | activity of AMP-activated protein kinase, causes inhibition of mTOR, regulates cell |
| | polarity, inhibits the cell cycle, and activates p53. Somatic mutations in this gene are |
| | associated with a history of smoking and KRAS mutation in NSCLC patients. The |
| | frequency of STK11 mutation in lung adenocarcinomas ranges from 7%-30%. STK11 |
| | loss may play a role in development of metastatic disease in lung cancer patients. |
| | Mutations of this gene also drive progression of HPV-induced dysplasia to invasive, |
| | cervical cancer and hence STK11 status may be exploited clinically to predict the |
| | likelihood of disease recurrence. Germline mutations in STK11 are associated with |
| | Peutz-Jeghers syndrome which is characterized by early onset hamartomatous gastro- |
| | intestinal polyps and increased risk of breast, colon, gastric and ovarian cancer. |
| TLE3 | TLE3 is a member of the transducin-like enhancer of split (TLE) family of proteins that |
| | have been implicated in tumorigenesis. It acts downstream of APC and beta-catenin to |
| | repress transcription of a number of oncogenes, which influence growth and |
| | microtubule stability. Studies indicate that TLE3 expression is associated with response |
| | to taxane therapy. |
| TOP2A | TOPOIIA is an enzyme that alters the supercoiling of double-stranded DNA and allows |
| | chromosomal segregation into daughter cells. Due to its essential role in DNA |
| | synthesis and repair, and frequent overexpression in tumors, TOPOIIA is an ideal |
| | target for antineoplastic agents. Amplification of TOPOIIA with or without HER2 co- |
| | amplification, as well as high protein expression of TOPOIIA, have been associated |
| | with benefit from anthracycline based therapy. |
| TOPO1 | Topoisomerase I is an enzyme that alters the supercoiling of double-stranded DNA. |
| | TOPOI acts by transiently cutting one strand of the DNA to relax the coil and extend |
| | the DNA molecule. Expression of TOPOI has been associated with response to TOPOI |
| | inhibitors including irinotecan and topotecan. |
| TP53 | TP53, or p53, plays a central role in modulating response to cellular stress through |
| | transcriptional regulation of genes involved in cell-cycle arrest, DNA repair, apoptosis, |
| | and senescence. Inactivation of the p53 pathway is essential for the formation of the |
| | majority of human tumors. Mutation in p53 (TP53) remains one of the most commonly |
| | described genetic events in human neoplasia, estimated to occur in 30-50% of all |
| | cancers. Generally, presence of a disruptive p53 mutation is associated with a poor |
| | prognosis in all types of cancers, and diminished sensitivity to radiation and |
| | chemotherapy. In addition, various clinical trials (on www.clinicaltrials.gov) |
| | investigating agents which target p53's downstream or upstream effectors may have |
| | clinical utility depending on the p53 status. For example, for p53 mutated patients, |
| | Chk1 inhibitors in advanced cancer and Wee1 inhibitors in ovarian cancer have been |
| | investigated. For p53 wildtype patients with sarcoma, mdm2 inhibitors have been |
| | investigated. Germline p53 mutations are associated with the Li-Fraumeni syndrome |
| | (LFS) which may lead to early-onset of several forms of cancer currently known to |
| | occur in the syndrome, including sarcomas of the bone and soft tissues, carcinomas of |
| | the breast and adrenal cortex (hereditary adrenocortical carcinoma), brain tumors and |
| | acute leukemias. |
| TS | Thymidylate synthase (TS) is an enzyme involved in DNA synthesis that generates |
| | thymidine monophosphate (dTMP), which is subsequently phosphorylated to |
| | thymidine triphosphate for use in DNA synthesis and repair. Low levels of TS are |
| | predictive of response to fluoropyrimidines and other folate analogues. |
| TUBB3 | Class III β-Tubulin (TUBB3) is part of a class of proteins that provide the framework |
| | for microtubules, major structural components of the cytoskeleton. Due to their |
| | importance in maintaining structural integrity of the cell, microtubules are ideal targets |
| | for anti-cancer agents. Low expression of TUBB3 is associated with potential clinical |
| | benefit to taxane therapy. |
| VHL | VHL or von Hippel-Lindau gene encodes for tumor suppressor protein pVHL, which |
| | polyubiquitylates hypoxia-inducible factor. Absence of pVHL causes stabilization of |
| | HIF and expression of its target genes, many of which are important in regulating |
| | angiogenesis, cell growth and cell survival. VHL somatic mutation has been seen in 20- |
| | 70% of patients with sporadic clear cell renal cell carcinoma (ccRCC) and the mutation |
| | may imply a poor prognosis, adverse pathological features, and increased tumor grade |
| | or lymph-node involvement. Renal cell cancer patients with a ‘loss of function’ |
| | mutation in VHL show a higher response rate to therapy (bevacizumab or sorafenib) |
| | than is seen in patients with wild type VHL. Germline mutations in VHL cause von |
| | Hippel-Lindau syndrome, associated with clear-cell renal-cell carcinomas, central |
| | nervous system hemangioblastomas, pheochromocytomas and pancreatic tumors. |

Table 5 shows exemplary MI molecular profiles for various tumor lineages. In the table, the lineage is shown in the column “Tumor Type.” The remaining columns show various biomarkers that can be assessed using the indicated methodology (i.e., immunohistochemistry (IHC), ISH or other techniques). One of skill will appreciate that similar methodology can be employed as desired. For example, other suitable protein analysis methods can be used instead of IHC, other suitable nucleic acid analysis methods can be used instead of ISH (e.g., that assess copy number and/or rearrangements, translocations and the like), and other suitable nucleic acid analysis methods can be used instead of fragment analysis. Similarly, FISH and CISH are generally interchangeable and the choice may be made based upon probe availability, resources, and the like. Tables 6-10 present panels of genes that can be assessed as part of the MI molecular profiles using Next Generation Sequencing (NGS) analysis. One of skill will appreciate that other nucleic acid analysis methods can be used instead of NGS analysis, e.g., other sequencing, hybridization (e.g., microarray, Nanostring) and/or amplification (e.g., PCR based) methods.

Nucleic acid analysis may be performed to assess various aspects of a gene. For example, nucleic acid analysis can include, but is not limited to, mutational analysis, fusion analysis, variant analysis, splice variants, SNP analysis and gene copy number/amplification. Such analysis can be performed using any number of techniques described herein or known in the art, including without limitation sequencing (e.g., Sanger, Next Generation, pyrosequencing), PCR, variants of PCR such as RT-PCR, fragment analysis, and the like. NGS techniques may be used to detect mutations, fusions, variants and copy number of multiple genes in a single assay. Table 4 describes a number of biomarkers including genes bearing mutations that have been identified in various cancer lineages. Unless otherwise stated or obvious in context, a “mutation” as used herein may comprise any change in a gene as compared to its wild type, including without limitation a mutation, polymorphism, deletion, insertion, indels (i.e., insertions or deletions), substitution, translocation, fusion, break, duplication, amplification, repeat, or copy number variation. In an aspect, the invention provides a molecular profile comprising mutational analysis of one or more genes in any of Tables 7-10. In one embodiment, the genes are assessed using Next Generation sequencing methods, e.g., using a TruSeq/MiSeq/HiSeq/NexSeq system offered by Illumina Corporation or an Ion Torrent system from Life Technologies.

In preferred embodiments, the MI molecular profiles of the invention comprise high-throughput sequencing analysis. Exemplary analyses are listed in Tables 6-10. As desired, different analyses may be performed for different sets of genes. For example, Table 6 lists various genes that may be assessed for genomic stability (e.g., MSI and TMB), Table 7 lists various genes that may be assessed for point mutations and indels, Table 8 lists various genes that may be assessed for point mutations, indels and copy number variations, Table 9 lists various genes that may be assessed for gene fusions, and Table 10 lists genes that can be assessed for transcript variants. Gene fusion and transcript analysis may be performed by analysis of RNA transcripts as desired.

Table 5 provides various biomarker panels that can be assessed for the indicated tumor lineages. In preferred embodiments, the panels can comprise the NGS analyses in Tables 6-10. For example, in the NGS column in Table 5, the Mutation analysis can be performed on DNA using the panels in Tables 6-8, and Table 10 as desired, the CNA analysis can be performed on DNA using the panel in Table 8, and the Fusion analysis can be performed on RNA using the panels in Table 9. Table 11 presents a view of associations between the biomarkers assessed and various therapeutic agents. Such associations can be determined by correlating the biomarker assessment results with drug associations from sources such as the NCCN, literature reports and clinical trials. The columns headed “Agent” provide candidate agents (e.g., drugs) or biomarker status to be included in the report. In some cases, the agent comprises clinical trials that can be matched to a biomarker status. Where agents are indicated, the association of the agent with the indicated biomarker can included in the MI report. In certain cases, multiple biomarkers are associated with a given agent or agents. For example, carboplatin, cisplatin, oxaliplatin are associated with BRCA1, BRCA2 and ERCC1. Platform abbreviations are as used throughout the application, e.g., IHC: immunohistochemistry; CISH: colorimetric in situ hybridization; NGS: next generation sequencing; PCR: polymerase chain reaction; CNA: copy number alteration. The candidate agents may comprise those undergoing clinical trials, as indicated.

As described herein, the invention further provides a report comprising results of the molecular profiling and corresponding candidate treatments that are identified as likely beneficial or likely not beneficial.

TABLE 5
Molecular Profile and Report Parameters

| | | Next Generation | |
|---|---|---|---|
| Tumor Type | Immunohistochemistry (IHC) | Sequencing (NGS) | Other |
| Bladder | ERCC1, PD-L1, RRM1, TOP2A, | Mutation, CNA | HER2, TOP2A |
| | TS, TUBB3 | Analysis (DNA) | (CISH) |
| Breast | AR, ER, ERCC1, ERBB2 (Her2), | Mutation, CNA | |
| | PD-L1, PR, PTEN, TOPO1, TS | Analysis (DNA) | |
| Cancer of Unknown | ERCC1, PD-L1, RRM1, TOPO1, TS, | Mutation, CNA | |
| Primary | TUBB3 | Analysis (DNA) | |
| Cervix | ER, ERCC1, PD-L1, PR, RRM1, | Mutation, CNA | |
| | TOP2A, TOPO1, TS, TUBB3 | Analysis (DNA) | |
| Cholangiocarcinoma/ | ERCC1, ERBB2 (Her2), PD-L1, | Mutation, CNA | HER2 (CISH) |
| Hepatobiliary | RRM1, TOPO1, TS, TUBB3 | Analysis (DNA); | |
| | | Fusion Analysis | |
| | | (RNA) | |
| Colorectal and Small | ERCC1, MLH1, MSH2, MSH6, | Mutation, CNA | |
| Intestinal | PD-L1, PMS2, PTEN, TOPO1, TS | Analysis (DNA) | |
| Endometrial | ER, ERCC1, MLH1, MSH2, MSH6, | Mutation, CNA | |
| | PMS2, PR, PD-L1, PTEN, RRM1, | Analysis (DNA) | |
| | TOP2A, TOPO1, TS, TUBB3 | | |
| Gastric | ERCC1, ERBB2 (Her2), MLH1, | Mutation, CNA | HER2 (CISH) |
| | MSH2, MSH6, PMS2, PD-L1, | Analysis (DNA) | |
| | TOP2A, TOPO1, TS, TUBB3 | | |
| GIST | PD-L1, PTEN | Mutation, CNA | |
| | | Analysis (DNA) | |
| Glioma | ERCC1, PD-L1, TOPO1 | Mutation, CNA | MGMT |
| | | Analysis (DNA); | Methylation |
| | | Fusion Analysis | (Pyrosequencing) |
| | | (RNA) | |
| Head & Neck | ERCC1, PD-L1, RRM1, TS, TUBB3 | Mutation, CNA | |
| | | Analysis (DNA) | |
| Kidney | ERCC1, PD-L1, RRM1, TOP2A, | Mutation, CNA | |
| | TUBB3 | Analysis (DNA) | |
| Melanoma | ERCC1, MGMT, PD-L1, TUBB3 | Mutation, CNA | |
| | | Analysis (DNA) | |
| Merkel Cell | ERCC1, TOPO1, TOP2A, PD-L1 | Mutation, CNA | |
| | | Analysis (DNA) | |
| Neuroendocrine/ | ERCC1, PD-L1, MGMT, TOP2A, | Mutation, CNA | |
| Small Cell Lung | TS | Analysis (DNA) | |
| Non-Serous | ER, ERCC1, MLH1, MSH2, MSH6, | Mutation, CNA | |
| Epithelial Ovarian | PMS2, PD-L1, PR, RRM1, TOP2A, | Analysis (DNA) | |
| | TOPO1, TUBB3 | | |
| Non-Small Cell | ALK, PD-L1, PTEN, RRM1, | Mutation, CNA | |
| Lung | TOPO1, TS, TUBB3 | Analysis (DNA); | |
| | | Fusion Analysis | |
| | | (RNA) | |
| Ovarian | ER, ERCC1, PD-L1, PR, RRM1, | Mutation, CNA | |
| | TOP2A, TOPO1, TUBB3 | Analysis (DNA) | |
| Pancreatic | ERCC1, MLH1, MSH2, MSH6, PD-L1, | Mutation, CNA | |
| | PMS2, RRM1, TOPO1, TS, | Analysis (DNA) | |
| | TUBB3 | | |
| Prostate | AR, ERCC1, PD-L1, TUBB3 | Mutation, CNA | |
| | | Analysis (DNA) | |
| Sarcoma | ERCC1, MGMT, PD-L1, RRM1, | Mutation, CNA | |
| | TOP2A, TOPO1, TUBB3 | Analysis (DNA) | |
| Thyroid | ERCC1, PD-L1, TOP2A | Mutation, CNA | |
| | | Analysis (DNA); | |
| | | Fusion Analysis | |
| | | (RNA) | |
| Other Tumors | ERCC1, PD-L1, RRM1, TOP2A, TS, | Mutation, CNA | |
| | TUBB3 | Analysis (DNA) | |

TABLE 6
Genomic Stability Testing (DNA)

| | |
|---|---|
| Microsatellite Instability (MSI) | Tumor Mutational Burden (TMB) |

TABLE 7
Point Mutations and Indels

| | | | | |
|---|---|---|---|---|
| ABI1 | CRLF2 | HOXC11 | MUC1 | RHOH |
| ABL1 | DDB2 | HOXC13 | MUTYH | RNF213 |
| ACKR3 | DDIT3 | HOXD11 | MYCL | RPL10 |
| | | | (MYCL1) | |
| AKT1 | DNM2 | HOXD13 | NBN | SEPT5 |
| AMER1 | DNMT3A | HRAS | NDRG1 | SEPT6 |
| (FAM123B) | | | | |
| AR | EIF4A2 | IKBKE | NKX2-1 | SFPQ |
| ARAF | ELF4 | INHBA | NONO | SLC45A3 |
| ATP2B3 | ELN | IRS2 | NOTCH1 | SMARCA4 |
| ATRX | ERCC1 | JUN | NRAS | SOCS1 |
| BCL11B | ETV4 | KAT6A | NUMA1 | SOX2 |
| | | (MYST3) | | |
| BCL2 | FAM46C | KAT6B | NUTM2B | SPOP |
| BCL2L2 | FANCF | KCNJ5 | OLIG2 | SRC |
| BCOR | FEV | KDM5C | OMD | SSX1 |
| BCORL1 | FOXL2 | KDM6A | P2RY8 | STAG2 |
| BRD3 | FOXO3 | KDSR | PAFAH1B2 | TAL1 |
| BRD4 | FOXO4 | KLF4 | PAK3 | TAL2 |
| BTG1 | FSTL3 | KLK2 | PATZ1 | TBL1XR1 |
| BTK | GATA1 | LASP1 | PAX8 | TCEA1 |
| C15orf65 | GATA2 | LMO1 | PDE4DIP | TCL1A |
| CBLC | GNA11 | LMO2 | PHF6 | TERT |
| CD79B | GPC3 | MAFB | PHOX2B | TFE3 |
| CDH1 | HEY1 | MAX | PIK3CG | TFPT |
| CDK12 | HIST1H3B | MECOM | PLAG1 | THRAP3 |
| CDKN2B | HIST1H4I | MED12 | PMS1 | TLX3 |
| CDKN2C | HLF | MKL1 | POU5F1 | TMPRSS2 |
| CEBPA | HMGN2P46 | MLLT11 | PPP2R1A | UBR5 |
| CHCHD7 | HNF1A | MN1 | PRF1 | VHL |
| CNOT3 | HOXA11 | MPL | PRKDC | WAS |
| COL1A1 | HOXA13 | MSN | RAD21 | ZBTB16 |
| COX6C | HOXA9 | MTCP1 | RECQL4 | ZRSR2 |

TABLE 8
Point Mutations, Indels and Copy Number Variations

| | | | | |
|---|---|---|---|---|
| ABL2 | CREB1 | FUS | MYC | RUNX1 |
| ACSL3 | CREB3L1 | GAS7 | MYCN | RUNX1T1 |
| ACSL6 | CREB3L2 | GATA3 | MYD88 | SBDS |
| ADGRA2 | CREBBP | GID4 | MYH11 | SDC4 |
| | | (C17orf39) | | |
| AFDN | CRKL | GMPS | MYH9 | SDHAF2 |
| AFF1 | CRTC1 | GNA13 | NACA | SDHB |
| AFF3 | CRTC3 | GNAQ | NCKIPSD | SDHC |
| AFF4 | CSF1R | GNAS | NCOA1 | SDHD |
| AKAP9 | CSF3R | GOLGA5 | NCOA2 | SEPT9 |
| AKT2 | CTCF | GOPC | NCOA4 | SET |
| AKT3 | CTLA4 | GPHN | NF1 | SETBP1 |
| ALDH2 | CTNNA1 | GRIN2A | NF2 | SETD2 |
| ALK | CTNNB1 | GSK3B | NFE2L2 | SF3B1 |
| APC | CYLD | H3F3A | NFIB | SH2B3 |
| ARFRP1 | CYP2D6 | H3F3B | NFKB2 | SH3GL1 |
| ARHGAP26 | DAXX | HERPUD1 | NFKBIA | SLC34A2 |
| ARHGEF12 | DDR2 | HGF | NIN | SMAD2 |
| ARID1A | DDX10 | HIP1 | NOTCH2 | SMAD4 |
| ARID2 | DDX5 | HMGA1 | NPM1 | SMARCB1 |
| ARNT | DDX6 | HMGA2 | NSD1 | SMARCE1 |
| ASPSCR1 | DEK | HNRNPA2B1 | NSD2 | SMO |
| ASXL1 | DICER1 | HOOK3 | NSD3 | SNX29 |
| ATF1 | DOT1L | HSP90AA1 | NT5C2 | SOX10 |
| ATIC | EBF1 | HSP90AB1 | NTRK1 | SPECC1 |
| ATM | ECT2L | IDH1 | NTRK2 | SPEN |
| ATP1A1 | EGFR | IDH2 | NTRK3 | SRGAP3 |
| ATR | ELK4 | IGF1R | NUP214 | SRSF2 |
| AURKA | ELL | IKZF1 | NUP93 | SRSF3 |
| AURKB | EML4 | IL2 | NUP98 | SS18 |
| AXIN1 | EMSY | IL21R | NUTM1 | SS18L1 |
| AXL | EP300 | IL6ST | PALB2 | STAT3 |
| BAP1 | EPHA3 | IL7R | PAX3 | STAT4 |
| BARD1 | EPHA5 | IRF4 | PAX5 | STAT5B |
| BCL10 | EPHB1 | ITK | PAX7 | STIL |
| BCL11A | EPS15 | JAK1 | PBRM1 | STK11 |
| BCL2L11 | ERBB2 | JAK2 | PBX1 | SUFU |
| | (HER2/NEU) | | | |
| BCL3 | ERBB3 (HER3) | JAK3 | PCM1 | SUZ12 |
| BCL6 | ERBB4 (HER4) | JAZF1 | PCSK7 | SYK |
| BCL7A | ERC1 | KDM5A | PDCD1 (PD1) | TAF15 |
| BCL9 | ERCC2 | KDR (VEGFR2) | PDCD1LG2 (PDL2) | TCF12 |
| BCR | ERCC3 | KEAP1 | PDGFB | TCF3 |
| BIRC3 | ERCC4 | KIAA1549 | PDGFRA | TCF7L2 |
| BLM | ERCC5 | KIF5B | PDGFRB | TET1 |
| BMPR1A | ERG | KIT | PDK1 | TET2 |
| BRCA1 | ESR1 | KLHL6 | PER1 | TFEB |
| BRCA2 | ETV1 | KMT2A (MLL) | PICALM | TFG |
| BRIP1 | ETV5 | KMT2C (MLL3) | PIK3CA | TFRC |
| BUB1B | ETV6 | KMT2D (MLL2) | PIK3R1 | TGFBR2 |
| CACNA1D | EWSR1 | KNL1 | PIK3R2 | TLX1 |
| CALR | EXT1 | KRAS | PIM1 | TNFAIP3 |
| CAMTAI | EXT2 | KTN1 | PML | TNFRSF14 |
| CANT1 | EZH2 | LCK | PMS2 | TNFRSF17 |
| CARD11 | EZR | LCP1 | POLE | TOP1 |
| CARS | FANCA | LGR5 | POT1 | TP53 |
| CASP8 | FANCC | LHFPL6 | POU2AF1 | TPM3 |
| CBFA2T3 | FANCD2 | LIFR | PPARG | TPM4 |
| CBFB | FANCE | LPP | PRCC | TPR |
| CBL | FANCG | LRIG3 | PRDM1 | TRAF7 |
| CBLB | FANCL | LRP1B | PRDM16 | TRIM26 |
| CCDC6 | FAS | LYL1 | PRKAR1A | TRIM27 |
| CCNB1IP1 | FBXO11 | MAF | PRRX1 | TRIM33 |
| CCND1 | FBXW7 | MALT1 | PSIP1 | TRIP11 |
| CCND2 | FCRL4 | MAML2 | PTCH1 | TRRAP |
| CCND3 | FGF10 | MAP2K1 | PTEN | TSC1 |
| | | (MEK1) | | |
| CCNE1 | FGF14 | MAP2K2 | PTPN11 | TSC2 |
| | | (MEK2) | | |
| CD274 (PDL1) | FGF19 | MAP2K4 | PTPRC | TSHR |
| CD74 | FGF23 | MAP3K1 | RABEP1 | TTL |
| CD79A | FGF3 | MCL1 | RAC1 | U2AF1 |
| CDC73 | FGF4 | MDM2 | RAD50 | USP6 |
| CDH11 | FGF6 | MDM4 | RAD51 | VEGFA |
| CDK4 | FGFR1 | MDS2 | RAD51B | VEGFB |
| CDK6 | FGFR1OP | MEF2B | RAF1 | VTI1A |
| CDK8 | FGFR2 | MEN1 | RALGDS | WDCP |
| CDKN1B | FGFR3 | MET | RANBP17 | WIF1 |
| CDKN2A | FGFR4 | MITF | RAP1GDS1 | WISP3 |
| CDX2 | FH | MLF1 | RARA | WRN |
| CHEK1 | FHIT | MLH1 | RB1 | WT1 |
| CHEK2 | FIP1L1 | MLLT1 | RBM15 | WWTR1 |
| CHIC2 | FLCN | MLLT10 | REL | XPA |
| CHN1 | FLI1 | MLLT3 | RET | XPC |
| CIC | FLT1 | MLLT6 | RICTOR | XPO1 |
| CIITA | FLT3 | MNX1 | RMI2 | YWHAE |
| CLP1 | FLT4 | MRE11 | RNF43 | ZMYM2 |
| CLTC | FNBP1 | MSH2 | ROS1 | ZNF217 |
| CLTCL1 | FOXA1 | MSH6 | RPL22 | ZNF331 |
| CNBP | FOXO1 | MSI2 | RPL5 | ZNF384 |
| CNTRL | FOXP1 | MTOR | RPN1 | ZNF521 |
| | FUBP1 | MYB | RPTOR | ZNF703 |

TABLE 9
Gene Fusions

| | | | | |
|---|---|---|---|---|
| AKT3 | ETV4 | MAST2 | NUTM1 | ROS1 |
| ALK | ETV5 | MSMB | PDGFRA | RSPO2 |
| ARHGAP26 | ETV6 | MUSK | PDGFRB | RSPO3 |
| AXL | EWSR1 | MYB | PIK3CA | TERT |
| BRAF | FGFR1 | NOTCH1 | PKN1 | TFE3 |
| BRD3 | FGFR2 | NOTCH2 | PPARG | TFEB |
| BRD4 | FGFR3 | NRG1 | PRKCA | THADA |
| EGFR | FGR | NTRK1 | PRKCB | TMPRSS2 |
| ERG | INSR | NTRK2 | RAF1 | |
| ESR1 | MAML2 | NTRK3 | RELA | |
| ETV1 | MAST1 | NUMBL | RET | |

TABLE 10
Variant Transcripts

EGFRvIII

TABLE 11
Therapeutic Agent - Biomarker Associations

| Agent | Biomarker | Platform |
|---|---|---|
| afatinib (assoc. in NSCLC only) | EGFR | NGS Mutation |
| | ERBB2 (Her2) | NGS Mutation |
| afatinib + cetuximab (combination assoc. in | EGFR T790M | NGS Mutation |
| NSCLC only) | | |
| alectinib, brigatinib, ceritinib | ALK | IHC; NGS Fusion Analysis |
| | | (RNA) |
| aspirin (assoc. in CRC only) | PIK3CA | NGS Mutation |
| avelumab (assoc. in Merkel cell only) | PD-L1 | IHC |
| cabozantinib | RET | NGS Fusion Analysis (RNA) |
| capecitabine, fluorouracil, pemetrexed | TS | IHC |
| carboplatin, cisplatin, oxaliplatin | ATM | NGS Mutation |
| | BRCA1 | NGS Mutation |
| | BRCA2 | NGS Mutation |
| | ERCC1 | IHC |
| cetuximab, panitumumab (assoc. in CRC | BRAF | NGS Mutation |
| only) | | |
| | KRAS | NGS Mutation |
| | NRAS | NGS Mutation |
| | PIK3CA | NGS Mutation |
| | PTEN | IHC |
| cetuximab | EGFR | NGS CNA |
| crizotinib | ALK | IHC; NGS Mutation (DNA) |
| | | & Fusion Analysis (RNA) |
| | MET | NGS Mutation, CNA (DNA) |
| | ROS1 | NGS Fusion Analysis (RNA) |
| dabrafenib, cobimetinib, vemurafenib | BRAF | NGS Mutation |
| dacarbazine, temozolomide | MGMT | IHC |
| | MGMT-Methylation | Pyrosequencing |
| | IDH1 (assoc. in High | NGS Mutation |
| | Grade Glioma only) | |
| docetaxel, paclitaxel, nab-paclitaxel | TUBB3 | IHC |
| doxorubicin, liposomal-doxorubicin, | TOP2A | IHC |
| epirubicin | | |
| | | CISH (Breast only) |
| enzalutamide, bicalutamide | AR (assoc. in TNBC | IHC |
| | only) | |
| erlotinib, gefitinib (assoc. in NSCLC only) | EGFR | NGS Mutation |
| | KRAS | NGS Mutation |
| | PIK3CA | NGS Mutation |
| | cMET | NGS CNA (DNA) |
| | PTEN | IHC |
| everolimus, temsirolimus | ER (assoc. in Breast | IHC |
| | only) | |
| | PIK3CA (excluding | NGS Mutation |
| | CRC) | |
| exemestane + everolimus, fulvestrant, | ER | IHC |
| palbociclib combination therapy | | |
| | ESRI | NGS Mutation |
| gemcitabine | RRM1 (excluding | IHC |
| | Breast) | |
| hormone therapies | AR | IHC |
| | ER | IHC |
| | PR | IHC |
| imatinib | KIT | NGS Mutation |
| | PDGFRA | NGS Mutation |
| irinotecan | TOPO1 | IHC |
| topotecan (excluding Breast, CRC, NSCLC) | | |
| lapatinib, neratinib, pertuzumab, T-DM1 | ERBB2 (Her2) | NGS CNA (DNA) |
| mitomycin-c | BRCA1 | NGS Mutation |
| | BRCA2 | |
| atezolizumab, nivolumab, pembrolizumab | PD-L1 | IHC |
| (assoc. in Bladder, CUP, Gastric, Kidney, | | |
| Melanoma, NSCLC only) | | |
| nivolumab, pembrolizumab | MSI | NGS Mutation |
| niraparib, olaparib, rucaparib | ATM (assoc. in | NGS Mutation |
| | Prostate only) | |
| | BRCA1 | |
| | BRCA2 | |
| osimertinib (assoc. in NSCLC only) | EGFR T790M | NGS Mutation |
| palbociclib, abemaciclib, ribociclib (assoc. in | ER | IHC |
| Breast only) | | |
| | ERBB2 (Her2) | IHC |
| sunitinib (assoc. in GIST only) | KIT | NGS |
| trametinib (assoc. in Melanoma and Lung) | BRAF | NGS |
| trastuzumab | ERBB2 (HER2) | CISH, IHC, NGS Mutation |
| | | (NSCLC only), CNA (DNA) |
| | PTEN (assoc. in | IHC |
| | Breast only) | |
| | PIK3CA (assoc. in | NGS Mutation |
| | Breast only) | |
| vandetanib | RET | NGS Mutation (DNA) & |
| | | Fusion Analysis (RNA) |

With regard to Table 11, cetuximab/panitumumab, vemurafenib/dabrafenib, and trametinib may be reported in combination for CRC. Hormone therapies may include: tamoxifen, toremifene, fulvestrant, letrozole, anastrozole, exemestane, megestrol acetate, leuprolide, goserelin, bicalutamide, flutamide, abiraterone, enzalutamide, triptorelin, abarelix, degarelix.

The biomarker—treatment associations can follow certain rules. The rules comprise a predicted likelihood of benefit or lack of benefit of a certain treatment for the cancer given an assessment of one or more biomarker. Exemplary biomarker—treatment association rules that can be used in the systems and methods of the invention are presented in any of International Patent Publications WO/2007/137187 (Int'l Appl. No. PCT/US2007/069286, published Nov 29. 2007; WO/2010/045318 (Int'l Appl. No. PCT/US2009/060630), published Apr. 22 2010; WO12010/093465 (Int'l Appl. No. PCT/US2010/100407), published Aug. 19, 2010; WO/2012/170715 (Int'l Appl. No. PCT/US2012/041393), published Dec. 13, 2012; WO/2014/089241 (Int'l Appl. No. PCT/US2013/073184), published Jun, 12, 2014; WO/2011/056688 (Intl Appl. No. PCT/US2010/054366), published May 12. 2011; WO/2012/092336 (Int'l Appl. No. PCT/US2011/067527), published Jul. 5, 2012; WO/2015/116868 (Int'l Appl. No. PCT/US2015/013618), published Aug. 6-2015; WO/2017/053915 (Int'l Appl. No. PCT/US2016/053614), published Mar. 30, 2017; and WO/2016/141169 (Int'l Appl. No. PCT/US2016/020657) published Sep, 9, 2016: each of which publications is incorporated by reference herein in its entirety, Based on the molecular profiling results, the rules may provide a predicted benefit level and an evidence level, and list of references for each biomarker-drug association rule. In embodiments of the invention, the benefit level is ranked From 1-5, wherein the levels indicate the predicted strength of the biomarker-drug association based on the indicated evidence. Relevant published studies can be evaluated using the U S. Preventive Services Task Force (“USPSTF”) grading scheme for study design and validity in some embodiments, the benefit level predicted for the agent corresponds to the following:
1: Expected benefit.
2: Expected reduced benefit.
3: Expected lack of benefit.
4: No data is available.
5: Data is available but no expected benefit or lack of benefit reported because the biomarker in this case is the not principal driver of that specific rule.

The evidence level may correspond to the following:
1: Very high level of evidence. For example, the treatment comprises the standard of care.
2: High level of evidence but perhaps insufficient to be considered for standard of care.
3: Weaker evidence—fewer publications or clinical studies, or perhaps some controversial evidence.

Any of the biomarker assays herein, including without limitation those listed in any of Tables 2-12, e.g., Table 4, Table 5, Table 6, Table 7, Table 8, Table 9, Table 10, Table 11, Table 12, or any useful combination thereof, can be performed individually as desired. Additional biomarkers can also be made available for individual testing, e.g., selected from any of International Patent Publications WO/2007/137187 (Int'l Appl. No. PCT/US2007/069286), published Nov. 29, 2007; WO/2010/045318 (Int'l Appl. No. PCT/US2009/060630), published Apr. 22, 2010; WO/2010/093465 (Int'l Appl. No. PCT/US2010/000407), published Aug. 19, 2010; WO/2012/170715 (Int'l Appl. No. PCT/US2012/041393), published Dec. 13, 2012; WO/2014/089241 (Int'l Appl. No. PCT/US2013/073184), published Jun. 12, 2014; WO/2011/056688 (Int'l Appl. No. PCT/US2010/054366), published May 12, 2011; WO/2012/092336 (Int'l Appl. No. PCT/US2011/067527), published Jul. 5, 2012; WO/2015/116868 (Int'l Appl. No. PCT/US2015/013618), published Aug. 6, 2015; WO/2017/053915 (Int'l Appl. No. PCT/US2016/053614), published Mar. 30, 2017; and WO/2016/141169 (Int'l Appl. No. PCT/US2016/020657), published Sep. 9, 2016; each of which publications is incorporated by reference herein in its entirety. One of skill will appreciate that any combination of the individual biomarker assays could be performed. In some embodiments, a selection of individual tests is made when insufficient tumor sample is available for performing all molecular profiling tests in Table 5.

As non-limiting examples, ERCC1 is assessed according to the profiles of the invention, such as described in any of Table 5 or Table 11. Lack of ERCC1 expression, e.g., as determined by IHC, can indicate positive benefit for platinum compounds (cisplatin, carboplatin, oxaliplatin), and conversely positive expression of ERCC1 can indicate lack of benefit of these drugs. The presence of EGFRvIII may be assessed using expression analysis at the protein or mRNA level, e.g., by either IHC or PCR, respectively. Expression of EGFRvIII can suggest treatment with EGFR inhibitors. Mutational analysis can be performed for IDH2, e.g., by Sanger sequencing, pyrosequencing or by next generation sequencing approaches. IDH2 mutations suggest the same therapy indications as IDH1 mutations, e.g., for decarbazine and temozolomide. In some cases, the analysis performed for each biomarker can depend on the lineage as desired. For example, EGFR IHC results may be assessed using H-SCORE for NSCLC but not other lineages.

Additional biomarkers that may be assessed according to the molecular profiling of the invention include BAP1 (BRCA1 Associated Protein-1 (Ubiquitin Carboxy-Terminal Hydrolase)), SETD2 (SET Domain Containing 2). In some embodiments of the invention, their expression is assessed at the protein and/or mRNA level. For example, IHC can be used to assess the protein expression of one or more of these biomarkers. PBRM1 and H3K36me3 may be assessed in kidney cancer, e.g., at the protein level such as by IHC. Molecular profiling of the invention can include at least one of TOP2A by CISH, Chromosome 17 by CISH, PBRM1 (PB1/BAF180) by IHC, BAP1 by IHC, SETD2 (ANTI-HISTONE H3) by IHC, MDM2 by CISH, Chromosome 12 by CISH, ALK by IHC, CTLA4 by IHC, CD3 by IHC, NY-ESO-1 by IHC, MAGE-A by IHC, TP by IHC, and EGFR by CISH.

### Nucleic Acid Sequence Analysis

The invention provides molecular profile for a cancer which comprises sequence analysis of panels of genes and other desired genetic loci. Sequence analysis can be used to detect any change in a gene as compared to its wild type, including without limitation a mutation, polymorphism, deletion, insertion, indels (i.e., insertions or deletions), substitution, translocation, fusion, break, duplication, amplification, repeat, or copy number variation. In some embodiments, the panel of genes is selected from any one of Tables 6-10 as described herein. For example, the molecular profile may comprise sequence analysis of at least one, e.g., at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45 or 46, of ABL1, AKT1, ALK, APC, ATM, BRAF, BRCA1, BRCA2, CDH1, CSF1R, CTNNB1, EGFR, ERBB2 (HER2), ERBB4 (HER4), FBXW7, FGFR1, FGFR2, FLT3, GNA11, GNAQ, GNAS, HNF1A, HRAS, IDH1, JAK2, JAK3, KDR (VEGFR2), KIT (cKIT), KRAS, MET (cMET), MPL, NOTCHI, NPM1, NRAS, PDGFRA, PIK3CA, PTEN, PTPN11, RB1, RET, SMAD4, SMARCB1, SMO, STK11, TP53, and VHL. The status of the genes can be linked to drug efficacy (e.g., predicted benefit or lack of benefit) or clinical trial enrollment as desired. See, e.g., Table 11.

The molecular profile may comprise analysis of at least one, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, 140, or all of ABIl, ABL1, ACKR3, AKT1, AMERI (FAM123B), AR, ARAF, ATP2B3, ATRX, BCL11B, BCL2, BCL2L2, BCOR, BCORL1, BRD3, BRD4, BTG1, BTK, C15orf65, CBLC, CD79B, CDH1, CDK12, CDKN2B, CDKN2C, CEBPA, CHCHD7, CNOT3, COL1A1, COX6C, CRLF2, DDB2, DDIT3, DNM2, DNMT3A, EIF4A2, ELF4, ELN, ERCC1, ETV4, FAM46C, FANCF, FEV, FOXL2, FOXO3, FOXO4, FSTL3, GATA1, GATA2, GNA11, GPC3, HEYl, HIST1H3B, HIST1H4I, HLF, HMGN2P46, HNF1A, HOXA11, HOXA13, HOXA9, HOXC11, HOXC13, HOXD11, HOXD13, HRAS, IKBKE, INHBA, IRS2, JUN, KAT6A (MYST3), KAT6B, KCNJ5, KDMSC, KDM6A, KDSR, KLF4, KLK2, LASP1, LMO1, LMO2, MAFB, MAX, MECOM, MED12, MKL1, MLLT11, MN1, MPL, MSN, MTCP1, MUC1, MUTYH, MYCL (MYCL1), NBN, NDRG1, NKX2-1, NONO, NOTCHI, NRAS, NUMA1, NUTM2B, OLIG2, OMD, P2RY8, PAFAH1B2, PAK3, PATZ1, PAX8, PDE4DIP, PHF6, PHOX2B, PIK3CG, PLAG1, PMS1, POU5F1, PPP2R1A, PRF1, PRKDC, RAD21, RECQL4, RHOH, RNF213, RPL10, SEPT5, SEPT6, SFPQ, SLC45A3, SMARCA4, SOCS1, SOX2, SPOP, SRC, SSX1, STAG2, TAL1, TAL2, TBL1XR1, TCEA1, TCL1A, TERT, TFE3, TFPT, THRAP3, TLX3, TMPRSS2, UBR5, VHL, WAS, ZBTB16 and ZRSR2. Such genes can be assessed, e.g., for point mutations and indels, or other characteristics as desired. The molecular profile may comprise analysis of at least one, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, 140, 150, 160, 170, 180, 190, 200, 250, 300, 350, 400 or all, of ABL2, ACSL3, ACSL6, AFF1, AFF3, AFF4, AKAP9, AKT2, AKT3, ALDH2, ALK, APC, ARFRP1, ARHGAP26, ARHGEF12, ARID1A, ARID2, ARNT, ASPSCR1, ASXL1, ATF1, ATIC, ATM, ATP1A1, ATR, AURKA, AURKB, AXIN1, AXL, BAP1, BARD1, BCL10, BCL11A, BCL2L11, BCL3, BCL6, BCL7A, BCL9, BCR, BIRC3, BLM, BMPR1A, BRAF, BRCA1, BRCA2, BRIP1, BUB1B, C11orf30 (EMSY), C2orf44, CACNA1D, CALR, CAMTA1, CANT1, CARD11, CARS, CASC5, CASP8, CBFA2T3, CBFB, CBL, CBLB, CCDC6, CCNB1IP1, CCND1, CCND2, CCND3, CCNE1, CD274 (PDL1), CD74, CD79A, CDC73, CDH11, CDK4, CDK6, CDK8, CDKN1B, CDKN2A, CDX2, CHEKI, CHEK2, CHIC2, CHN1, CIC, CIITA, CLP1, CLTC, CLTCL1, CNBP, CNTRL, COPB1, CREB1, CREB3L1, CREB3L2, CREBBP, CRKL, CRTC1, CRTC3, CSF1R, CSF3R, CTCF, CTLA4, CTNNA1, CTNNB1, CYLD, CYP2D6, DAXX, DDR2, DDX10, DDX5, DDX6, DEK, DICERI, DOT1L, EBF1, ECT2L, EGFR, ELK4, ELL, EML4, EP300, EPHA3, EPHA5, EPHB1, EPS15, ERBB2 (HER2), ERBB3 (HER3), ERBB4 (HER4), ERC1, ERCC2, ERCC3, ERCC4, ERCC5, ERG, ESR1, ETV1, ETV5, ETV6, EWSR1, EXT1, EXT2, EZH2, EZR, FANCA, FANCC, FANCD2, FANCE, FANCG, FANCL, FAS, FBXO11, FBXW7, FCRL4, FGF10, FGF14, FGF19, FGF23, FGF3, FGF4, FGF6, FGFR1, FGFR1OP, FGFR2, FGFR3, FGFR4, FH, FHIT, FIP1L1, FLCN, FLIl, FLT1, FLT3, FLT4, FNBP1, FOXA1, FOXO1, FOXP1, FUBP1, FUS, GAS7, GATA3, GID4 (C17orf39), GMPS, GNA13, GNAQ, GNAS, GOLGA5, GOPC, GPHN, GPR124, GRIN2A, GSK3B, H3F3A, H3F3B, HERPUDI, HGF, HIP1, HMGA1, HMGA2, HNRNPA2B1, HOOK3, HSP90AA1, HSP90AB1, IDH1, IDH2, IGF1R, IKZF1, IL2, IL21R, IL6ST, IL7R, IRF4, ITK, JAK1, JAK2, JAK3, JAZF1, KDM5A, KDR (VEGFR2), KEAP1, KIAA1549, KIF5B, KIT, KLHL6, KMT2A (MLL), KMT2C (MLL3), KMT2D (MLL2), KRAS, KTN1, LCK, LCP1, LGR5, LHFP, LIFR, LPP, LRIG3, LRP1B, LYL1, MAF, MALT1, MAML2, MAP2K1, MAP2K2, MAP2K4, MAP3K1, MCL1, MDM2, MDM4, MDS2, MEF2B, MEN1, MET (cMET), MITF, MLF1, MLH1, MLLT1, MLLT10, MLLT3, MLLT4, MLLT6, MNX1, MRE11A, MSH2, MSH6, MSJ2, MTOR, MYB, MYC, MYCN, MYD88, MYH11, MYH9, NACA, NCKIPSD, NCOA1, NCOA2, NCOA4, NF1, NF2, NFE2L2, NFIB, NFKB2, NFKBIA, NIN, NOTCH2, NPM1, NR4A3, NSD1, NT5C2, NTRK1, NTRK2, NTRK3, NUP214, NUP93, NUP98, NUTM1, PALB2, PAX3, PAX5, PAX7, PBRM1, PBX1, PCM1, PCSK7, PDCD1 (PD1), PDCD1LG2 (PDL2), PDGFB, PDGFRA, PDGFRB, PDK1, PER1, PICALM, PIK3CA, PIK3R1, PIK3R2, PIM1, PML, PMS2, POLE, POT1, POU2AF1, PPARG, PRCC, PRDM1, PRDM16, PRKAR1A, PRRX1, PSIP1, PTCH1, PTEN, PTPN11, PTPRC, RABEP1, RAC1, RAD50, RAD51, RAD51B, RAF1, RALGDS, RANBP17, RAP1GDS1, RARA, RB1, RBM15, REL, RET, RICTOR, RMJ2, RNF43, ROS1, RPL22, RPL5, RPN1, RPTOR, RUNX1, RUNX1T1, SBDS, SDC4, SDHAF2, SDHB, SDHC, SDHD, SEPT9, SET, SETBP1, SETD2, SF3B1, SH2B3, SH3GL1, SLC34A2, SMAD2, SMAD4, SMARCB1, SMARCE1, SMO, SNX29, SOX10, SPECCI, SPEN, SRGAP3, SRSF2, SRSF3, SS18, SS18L1, STAT3, STAT4, STAT5B, STIL, STK11, SUFU, SUZ12, SYK, TAF15, TCF12, TCF3, TCF7L2, TET1, TET2, TFEB, TFG, TFRC, TGFBR2, TLX1, TNFAIP3, TNFRSF14, TNFRSF17, TOP1, TP53, TPM3, TPM4, TPR, TRAF7, TRIM26, TRIM27, TRIM33, TRIP11, TRRAP, TSC1, TSC2, TSHR, TTL, U2AF1, USP6, VEGFA, VEGFB, VTI1A, WHSC1, WHSC1L1, WIF1, WISP3, WRN, WT1, WWTR1, XPA, XPC, XPO1, YWHAE, ZMYM2, ZNF217, ZNF331, ZNF384, ZNF521 and ZNF703. Such genes can be assessed, e.g., for point mutations, indels and copy number, or other characteristics as desired. The molecular profile may comprise analysis of at least one, e.g., 1, 2, 3, 4, 5, 6, 7 or 8 of ALK, BRAF, NTRK1, NTRK2, NTRK3, RET, ROS1 and RSPO3. Such genes can be assessed for gene fusions or other characteristics as desired. The molecular profile may comprise analysis of EGFR vIII and/or MET Exon 14 Skipping. Such analysis may include identification of variant transcripts. In some embodiments, all genes listed in Tables 6-10 are analyzed as indicated in the table headers. The analysis can be used to determine MSI, TMB, or both for the tumor. NGS sequencing may be used to perform such analysis in a high throughput manner. Any useful combinations such as those listed in this paragraph may be assessed by sequence analysis.

In an embodiment, the plurality of genes and/or gene products comprises sequence analysis of at least one, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56,57 or 58, of ABL1, AKT1, ALK, APC, AR, ARAF, ATM, BAP1, BRAF, BRCA1, BRCA2, CDK4, CDKN2A, CHEKI, CHEK2, CSF1R, CTNNB1, DDR2, EGFR, ERBB2, ERBB3, FGFR1, FGFR2, FGFR3, FLT3, GNA11, GNAQ, GNAS, HRAS, IDH1, IDH2, JAK2, KDR, KIT, KRAS, MAP2K1 (MEK1), MAP2K2 (MEK2), MET, MLH1, MPL, NF1, NOTCHI, NRAS, NTRK1, PDGFRA, PDGFRB, PIK3CA, PTCH1, PTEN, RAF1, RET, ROS1, SMO, SRC, TP53, VHL, WT1. The genes assessed by sequence analysis may further comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 110, 120, 130, 140, 150, 160, 170, 180, 190, 200, 250, 300, 350, 400, 450, 500, or all genes, selected from the group consisting of ABI1, ABL2, ACSL3, ACSL6, AFF1, AFF3, AFF4, AKAP9, AKT2, AKT3, ALDH2, AMER1, AR, ARFRP1, ARHGAP26, ARHGEF12, ARID1A, ARID2, ARNT, ASPSCR1, ASXL1, ATF1, ATIC, ATP1A1, ATP2B3, ATR, ATRX, AURKA, AURKB, AXIN1, AXL, BARD1, BCL10, BCL11A, BCL11B, BCL2, BCL2L11, BCL2L2, BCL3, BCL6, BCL7A, BCL9, BCOR, BCORL1, BCR, BIRC3, BLM, BMPR1A, BRD3, BRD4, BRIP1, BTG1, BTK, BUB1B, C11orf30, C15orf21, C15orf55, C15orf65, C16orf75, C2orf44, CACNA1D, CALR, CAMTA1, CANT1, CARD11, CARS, CASC5, CASP8, CBFA2T3, CBFB, CBL, CBLB, CBLC, CCDC6, CCNB1IP1, CCND1, CCND2, CCND3, CCNE1, CD274, CD74, CD79A, CD79B, CDC73, CDH11, CDK12, CDK4, CDK6, CDK8, CDKN1B, CDKN2A, CDKN2B, CDKN2C, CDX2, CEBPA, CHCHD7, CHIC2, CHN1, CIC, CIITA, CLP1, CLTC, CLTCL1, CNBP, CNOT3, CNTRL, COL1A1, COPB1, COX6C, CREB1, CREB3L1, CREB3L2, CREBBP, CRKL, CRLF2, CRTC1, CRTC3, CSF3R, CTCF, CTLA4, CTNNA1, CXCR7, CYLD, CYP2D6, DAXX, DDB2, DDIT3, DDX10, DDX5, DDX6, DEK, DICER1, DNM2, DNMT3A, DOT1L, DUX4, EBF1, ECT2L, EIF4A2, ELF4, ELK4, ELL, ELN, EML4, EP300, EPHA3, EPHA5, EPHB1, EPS15, ERC1, ERCC1, ERCC2, ERCC3, ERCC4, ERCC5, ERG, ESR1, ETV1, ETV4, ETV5, ETV6, EWSR1, EXT1, EXT2, EZH2, EZR, FAM123B, FAM22A, FAM22B, FAM46C, FANCA, FANCC, FANCD2, FANCE, FANCF, FANCG, FANCL, FAS, FBXO11, FCGR2B, FCRL4, FEV, FGF10, FGF14, FGF19, FGF23, FGF3, FGF4, FGF6, FGFR1OP, FGFR3, FGFR4, FH, FHIT, FIP1L1, FLCN, FLIl, FLT1, FLT4, FNBP1, FOXA1, FOXL2, FOXO1, FOXO3, FOXO4, FOXP1, FSTL3, FUBP1, FUS, GAS7, GATA1, GATA2, GATA3, GID4, GMPS, GNA13, GOLGA5, GOPC, GPC3, GPHN, GPR124, GRIN2A, GSK3B, H3F3A, H3F3B, HERPUDI, HEYl, HGF, HIP1, HIST1H3B, HIST1H4I, HLF, HMGA1, HMGA2, HNRNPA2B1, HOOK3, HOXA11, HOXA13, HOXA9, HOXC11, HOXC13, HOXD11, HOXD13, HSP90AA1, HSP90AB1, IGF1R, IKBKE, IKZF1, IL2, IL21R, IL6ST, IL7R, INHBA, IRF4, IRS2, ITK, JAK1, JAZF1, JUN, KAT6A, KCNJ5, KDM5A, KDM5C, KDM6A, KDSR, KEAP1, KIAA1549, KIF5B, KLF4, KLHL6, KLK2, KTN1, LASP1, LCK, LCP1, LGR5, LHFP, LIFR, LMO1, LMO2, LPP, LRIG3, LRP1B, LYL1, MAF, MAFB, MALT1, MAML2, MAP2K1 (MEK1), MAP2K2 (MEK2), MAP2K4, MAP3K1, MAX, MCL1, MDM2, MDM4, MDS2, MECOM, MED12, MEF2B, MEN1, MITF, MKL1, MLF1, MLL, MLL2, MLL3, MLLT1, MLLT10, MLLT11, MLLT3, MLLT4, MLLT6, MN1, MNX1, MRE11A, MSH2, MSH6, MSI2, MSN, MTCP1, MTOR, MUC1, MUTYH, MYB, MYC, MYCL1, MYCN, MYD88, MYH11, MYH9, MYST4, NACA, NBN, NCKIPSD, NCOA1, NCOA2, NCOA4, NDRG1, NF2, NFE2L2, NFIB, NFKB2, NFKBIA, NIN, NKX2-1, NONO, NOTCH2, NR4A3, NSD1, NT5C2, NTRK2, NTRK3, NUMA1, NUP214, NUP93, NUP98, OLIG2, OMD, P2RY8, PAFAH1B2, PAK3, PALB2, PATZ1, PAX3, PAX5, PAX7, PAX8, PBRM1, PBX1, PCM1, PCSK7, PDCD1, PDCD1LG2, PDE4DIP, PDGFB, PDGFRB, PDK1, PER1, PHF6, PHOX2B, PICALM, PIK3CG, PIK3R1, PIK3R2, PIM1, PLAG1, PML, PMS1, PMS2, POLE, POT1, POU2AF1, POU5F1, PPARG, PPP2R1A, PRCC, PRDM1, PRDM16, PRF1, PRKAR1A, PRKDC, PRRX1, PSIP1, PTCH1, PTPRC, RABEP1, RAC1, RAD21, RAD50, RAD51, RAD51L1, RALGDS, RANBP17, RAP1GDS1, RARA, RBM15, RECQL4, REL, RHOH, RICTOR, RNF213, RNF43, RPL10, RPL22, RPL5, RPN1, RPTOR, RUNDC2A, RUNX1, RUNx1T1, SBDS, SDC4, SDHAF2, SDHB, SDHC, SDHD, SEPT5, SEPT6, SEPT9, SET, SETBP1, SETD2, SF3B1, SFPQ, SFRS3, SH2B3, SH3GL1, SLC34A2, SLC45A3, SMAD2, SMARCA4, SMARCE1, SOCS1, SOX10, SOX2, SPECCI, SPEN, SPOP, SRC, SRGAP3, SRSF2, SS18, SS18L1, SSX1, SSX2, SSX4, STAG2, STAT3, STAT4, STAT5B, STIL, SUFU, SUZ12, SYK, TAF15, TAL1, TAL2, TBL1XR1, TCEA1, TCF12, TCF3, TCF7L2, TCL1A, TERT, TET1, TET2, TFE3, TFEB, TFG, TFPT, TFRC, TGFBR2, THRAP3, TLX1, TLX3, TMPRSS2, TNFAIP3, TNFRSF14, TNFRSF17, TOP1, TPM3, TPM4, TPR, TRAF7, TRIM26, TRIM27, TRIM33, TRIP11, TRRAP, TSC1, TSC2, TSHR, TTL, U2AF1, UBR5, USP6, VEGFA, VEGFB, VTI1A, WAS, WHSC1, WHSC1L1, WIF1, WISP3, WRN, WWTR1, XPA, XPC, XPO1, YWHAE, ZBTB16, ZMYM2, ZNF217, ZNF331, ZNF384, ZNF521, ZNF703 and ZRSR2. Any useful combinations such as those listed in this paragraph may be assessed by sequence analysis.

The genes assessed by sequence analysis may comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 110, 120, 130, 140, 150, 160, 170, 180, 190, 200, 250, 300, or all genes, selected from the group consisting of ABL1, ACVR1B, AKT1, AKT2, AKT3, ALK, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2, BCL2L1, BCL2L2, BCL6, BCOR, BCORL1, BCR, BRAF, BRAF, BRCA1, BRCA1, BRCA2, BRCA2, BRD4, BRIP1, BTG1, BTG2, BTK, C11orf30, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEKI, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EGFR, EP300, EPHA3, EPHB1, EPHB4, ERBB2, ERBB3, ERBB4, ERCC4, ERG, ERRFIl, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR1, FGFR2, FGFR2, FGFR3, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KIT, KLHL6, KMT2A (MLL), KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH2, MSH3, MSH6, MST1R, MTAP, MTOR, MUTYH, MYB, MYC, MYC, MYCL, MYCN, MYD88, NBN, NF1, NF2, NFE2L2, NFKBIA, NKX2-1, NOTCHI, NOTCH2, NOTCH2, NOTCH3, NPM1, NRAS, NT5C2, NTRK1, NTRK1, NTRK2, NTRK2, NTRK3, NUTM1, P2RY8, PALB2, PARK2, PARP1, PARP2, PARP3, PAX5, PBRM1, PDCD1, PDCD1LG2, PDGFRA, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RAF1, RARA, RARA, RB1, RBM10, REL, RET, RET, RICTOR, RNF43, ROS1, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, and ZNF703.

As noted, various cancers are characterized by chromosomal translocations and gene fusions. For example, acute lymphoblastic leukemia has been characterized by a number of kinase fusions. See, e.g., Table 12; G. Roberts et al., Targetable kinase-activating lesions in Ph-like acute lymphoblastic leukemia. N. Engl. J. Med. 371, 1005-1015 (2014), which reference is incorporated herein in its entirety. Crizotinib and imatinib target specific tyrosine kinases that form chimeric fusions. Crizotinib is FDA approved for ALK positive fusions in NSCLC and imatinib induces remission in leukemia patients that are positive for BCR-ABL fusions. In an embodiment, the molecular profile of the invention comprises sequence analysis to assess a gene fusion in at least one, e.g., at least 1, 2, 3, 4, 5, 6, 7, 8, 9, 10, 11 or 12, of ABL1, ABL2, CSF1R, PDGFRB, CRLF2, JAK2, EPOR, IL2RB, NTRK3, PTK2B, TSLP and TYK2. Kinase fusions and other gene fusions have been observed in a number of carcinomas. See, e.g., N. Stransky, E. Cerami, S. Schalm, J. L. Kim, C. Lengauer, The landscape of kinase fusions in cancer. Nat Commun 5, 4846 (2014), which reference is incorporated herein in its entirety. In another embodiment, sequence analysis is used to assess a gene fusion in at least one, e.g., at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52 or 53, of AKT3, ALK, ARHGAP26, AXL, BRAF, BRD3, BRD4, EGFR, ERG, ESR1, ETV1, ETV4, ETV5, ETV6, EWSR1, FGFR1, FGFR2, FGFR3, FGR, INSR, MAML2, MAST1, MAST2, MET, MSMB, MUSK, MYB, NOTCH1, NOTCH2, NRG1, NTRK1, NTRK2, NTRK3, NUMBL, NUTM1, PDGFRA, PDGFRB, PIK3CA, PKN1, PPARG, PRKCA, PRKCB, RAF1, RELA, RET, ROS1, RSPO2, RSPO3, TERT, TFE3, TFEB, THADA and TMPRSS2. Fusions with any desired number of these genes can be detected in carcinomas of various lineages. Similarly, a number of gene fusions have been detected in a variety of sarcomas. In an embodiment, sequence analysis is used to assess a gene fusion in at least one, e.g., at least 1, 2, 3, 4, 5, 6, 7, 8, 9, 10, 11, 12, 13, 14, 15, 16, 17, 18, 19, 20, 21, 22, 23, 24, 25 or 26, of ALK, CAMTA1, CCNB3, CIC, EPC, EWSR1, FKHR, FUS, GLI1, HMGA2, JAZF1, MEAF6, MKL2, NCOA2, NTRK3, PDGFB, PLAG1, ROS1, SS18, STAT6, TAF15, TCF12, TFE3, TFG, USP6 and YWHAE. Any desired number of fusions in these genes can be detected in various sarcomas. Additional gene fusions that can be detected as part of the molecular profiling of the invention are described in M. J. Annala, B. C. Parker, W. Zhang, M. Nykter, Fusion genes and their discovery using high throughput sequencing. Cancer Lett. 340, 192-200 (2013), which reference is incorporated herein in its entirety. Gene fusions can be detected by various technologies, including without limitation IHC (e.g., to detect mutant proteins produced by gene fusions), ISH, PCR (e.g., RT-PCR), microarrays and sequencing analysis. In an embodiment, the fusions are detected using Next Generation Sequencing technology.

TABLE 12
Kinase gene fusions

| Kinase Gene | 5′ Genes |
|---|---|
| ABL1 | ETV6, NUP214, RCSD1, RANBP2, SNX2, ZMIZ1 |
| ABL2 | PAG1, RCSD1 |
| CSF1R | SSBP2 |
| PDGFRB | EBF1, SSBP2, TNIP1, ZEB2 |
| CRLF2 | P2RY8 |
| JAK2 | ATF7IP, BCR, ETV6, PAX5, PPFIBP1, SSBP2, |
| | STRN3, TERF2, TPR |
| EPOR | IGH, IGK |
| IL2RB | MYH9 |
| NTRK3 | ETV6 |
| PTK2B | KDM6A, STAG2 |
| TSLP | IQGAP2 |
| TYK2 | MYB |

Various cancer genes disclosed in the COSMIC (Catalogue Of Somatic Mutations In Cancer) database (available at cancer.sanger.ac.uk/cancergenome/projects/cosmic/) can be assessed as well.

### Clinical Trial Connector

Thousands of clinical trials for therapies are underway in the United States, with several hundred of these tied to biomarker status. In an embodiment, the molecular intelligence molecular profiles of the invention include molecular profiling of markers that are associated with ongoing clinical trials, Thus, the molecular profile can be linked to clinical trials of therapies that are correlated to a subject's biomarker profile. The method can further comprise identifying trial location(s) to facilitate patient enrollment. The database of ongoing clinical trials can be obtained from publicly available online sources in the United States, or similar source in other locations. The molecular profiles generated by the methods of the invention can be linked to ongoing clinical trials and updated on a regular basis. e.g., daily, bi-weekly, weekly, monthly, or other appropriate time period.

Although significant advances in cancer treatment have been made in recent years, not all patients can be effectively treated within the standard of care paradigm. Many patients are eligible for clinical trials participation, yet less than 3 percent are actually enrolled in a trial, according to recent National Cancer Institute (NCI) statistics, The Clinical Trials Connector allows caregivers such as physicians to quickly identify and review global clinical trial opportunities in real-time that are molecularly targeted to each patient in embodiments, the Clinical Trials Connector has one or more of the following features: Examines thousands of open and enrolling clinical trials; Individualizes clinical trials based on molecular profiling as described herein; Includes interactive and customizable trial search filters by: Biomarker. Mechanism of action, Therapy, Phase of study, and other clinical factors (age, sex, etc). The Clinical Trials Connector can be a computer database that is accessed once molecular profiling results are available. In some embodiments, the database comprises the EmergingMed database (EmergingMed. New York, N.Y.). One of skill can identify appropriate clinical trials. e.g., by searching one or more publicly available databases using the various biomarkers of interest and determining whether the molecular profiling results indicated the patient meets eligibility criteria for the identified trials.

In an aspect, the invention provides a set of rules for matching of clinical trials to biomarker status as determined by the molecular profiling described herein. In some embodiments, the matching of clinical trials to biomarker status is performed using one or more pre-specified criteria: 1) Trials are matched based on the OFF NCCN Compendia drug/drug class associated with potential benefit by the molecular profiling rules; 2) Trials are matched based on biomarker driven eligibility requirement of the trial; and 3) Trials are matched based on the molecular profile of the patient, the biology of the disease and the associated signaling pathways. In the latter case, i.e. item 3, clinical trial matching may comprise further criteria as follows. First, for directly targetable markers, match trials with agents directly targeting the gene (e.g., FGFR results map to anti-FGFR therapy trials; ERBB2 results map to anti-HER2 agents, etc). In addition, for directly targetable markers, trial matching considers downstream markers under the following scenarios: a) a known resistance mechanism is available (e.g., cMET inhibitors for EGFR gene); b) clinical evidence associates the (mutated) biomarker with drugs targeting downstream pathways (e.g., mTOR inhibitors when PIK3CA is mutated); and c) active clinical trials are enrolling patients (with the biomarker aberration in the inclusion criteria) with drugs targeting the downstream pathways (e.g., SMO inhibitors for BCR-ABL mutation T3151). In the case of markers that are not directly targetable by a known therapeutic agent, trial matching may consider alternative, downstream markers (e.g., platinum agents for ATM gene; MEK inhibitors for GNAS/GNAQ/GNA11 mutation). The clinical trials that are matched may be identified based on results of “pathogenic,” “presumed pathogenic,” or variant of uncertain (or unknown) significance (“VUS”). In some embodiments, the decision to incorporate/associate a drug class with a biomarker mutation can further depend on one or more of the following: 1) Clinical evidence; 2) Preclinical evidence; 3) Understanding of the biological pathway affected by the biomarker; and 4) expert analysis. In some embodiments, the status of various biomarkers provided herein, e.g., in any of Tables 4-10 is linked to clinical trials using one or more of these criteria.

The guiding principle above can be used to identify classes of drugs that are linked to certain biomarkers. The biomarkers can be linked to various clinical trials that are studying these biomarkers, including without limitation requiring a certain biomarker status for clinical trial inclusion. Clinical trials studying the drug classes and/or specific agents listed can be matched to the biomarker. In an aspect, the invention provides a method of selecting a clinical trial for enrollment of a patient, comprising performing molecular profiling of one or more biomarker on a sample from the patient using the methods described herein. For example, the profiling can be performed for one on more biomarker in any of Tables 2-12 using the technique indicated in the table. The results of the profiling are matched to classes of drugs using the above criteria. Clinical trials studying members of the classes of drugs are identified. The patient is a potential candidate for the so-identified clinical trials.

### Report

In an embodiment, the methods of the invention comprise generating a molecular profile report. The report can be delivered to the treating physician or other caregiver of the subject whose cancer has been profiled. The report can comprise multiple sections of relevant information, including without limitation: 1) a list of the genes and/or gene products in the molecular profile; 2) a description of the molecular profile of the genes and/or gene products as determined for the subject; 3) a treatment associated with one or more of the genes and/or gene products in the molecular profile; and 4) and an indication whether each treatment is likely to benefit the patient, not benefit the patient, or has indeterminate benefit. The list of the genes and/or gene products in the molecular profile can be those presented herein for the molecular intelligence profiles of the invention. The description of the molecular profile of the genes and/or gene products as determined for the subject may include such information as the laboratory technique used to assess each biomarker (e.g., RT-PCR, FISH/CISH, IHC, PCR, FA/RFLP, NGS, etc) as well as the result and criteria used to score each technique. By way of example, the criteria for scoring a protein as positive or negative for IHC may comprise the amount of staining and/or percentage of positive cells, or criteria for scoring a mutation may be a presence or absence. The treatment associated with one or more of the genes and/or gene products in the molecular profile can be determined using a biomarker-drug association rule set such as in any of International Patent Publications WO/2007/137187 (Int'l Appl. No. PCT/US2007/069286), published Nov. 29, 2007; WO/2010/045318 (Int'l Appl. No. PCT/US2009/060630), published Apr. 22, 2010; WO/2010/093465 (Int'l Appl. No. PCT/US2010/000407), published Aug. 19, 2010; WO/2012/170715 (Int'l Appl. No. PCT/US2012/041393), published Dec. 13, 2012; WO/2014/089241 (Int'l Appl. No. PCT/US2013/073184), published Jun. 12, 2014; WO/2011/056688 (Int'l Appl. No. PCT/US2010/054366), published May 12, 2011; WO/2012/092336 (Int'l Appl. No. PCT/US2011/067527), published Jul. 5, 2012; WO/2015/116868 (Int'l Appl. No. PCT/US2015/013618), published Aug. 6, 2015; WO/2017/053915 (Int'l Appl. No. PCT/US2016/053614), published Mar. 30, 2017; and WO/2016/141169 (Int'l Appl. No. PCT/US2016/020657), published Sep. 9, 2016; each of which publications is incorporated by reference herein in its entirety. The indication whether each treatment is likely to benefit the patient, not benefit the patient, or has indeterminate benefit may be weighted. For example, a potential benefit may be a strong potential benefit or a lesser potential benefit. Such weighting can be based on any appropriate criteria, e.g., the strength of the evidence of the biomarker-treatment association, or the results of the profiling, e.g., a degree of over- or underexpression.

Various additional components can be added to the report as desired. In an embodiment, the report comprises a list having an indication of whether one or more of the genes and/or gene products in the molecular profile are associated with an ongoing clinical trial. The report may include identifiers for any such trials, e.g., to facilitate the treating physician's investigation of potential enrollment of the subject in the trial. In some embodiments, the report provides a list of evidence supporting the association of the genes and/or gene products in the molecular profile with the reported treatment. The list can contain citations to the evidentiary literature and/or an indication of the strength of the evidence for the particular biomarker-treatment association. In still another embodiment, the report comprises a description of the genes and/or gene products in the molecular profile. The description of the genes and/or gene products in the molecular profile may comprise without limitation the biological function and/or various treatment associations.

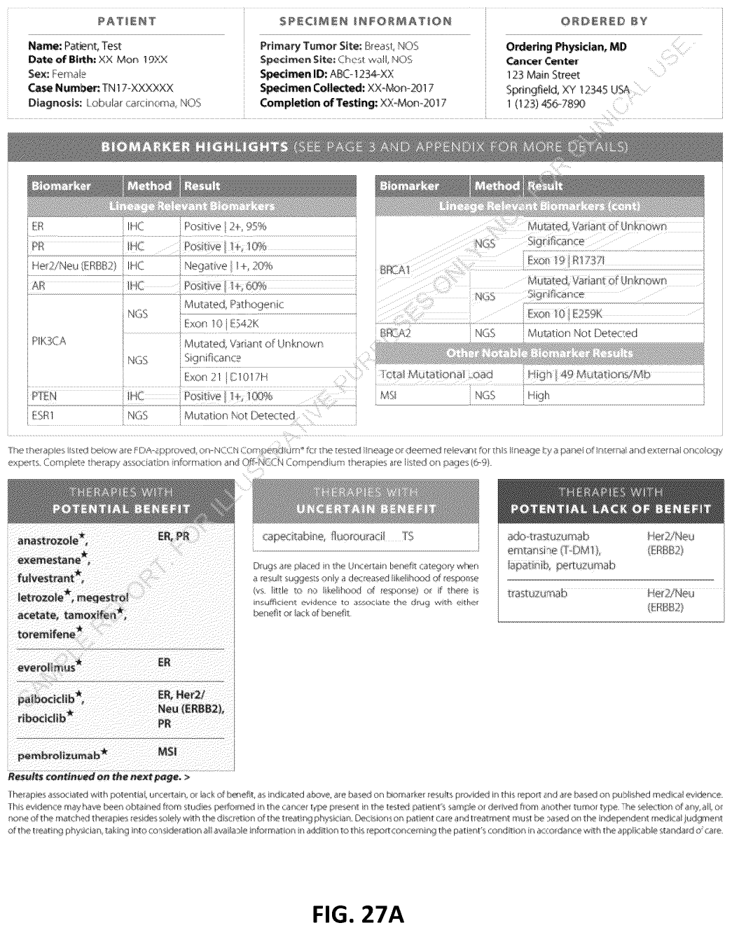

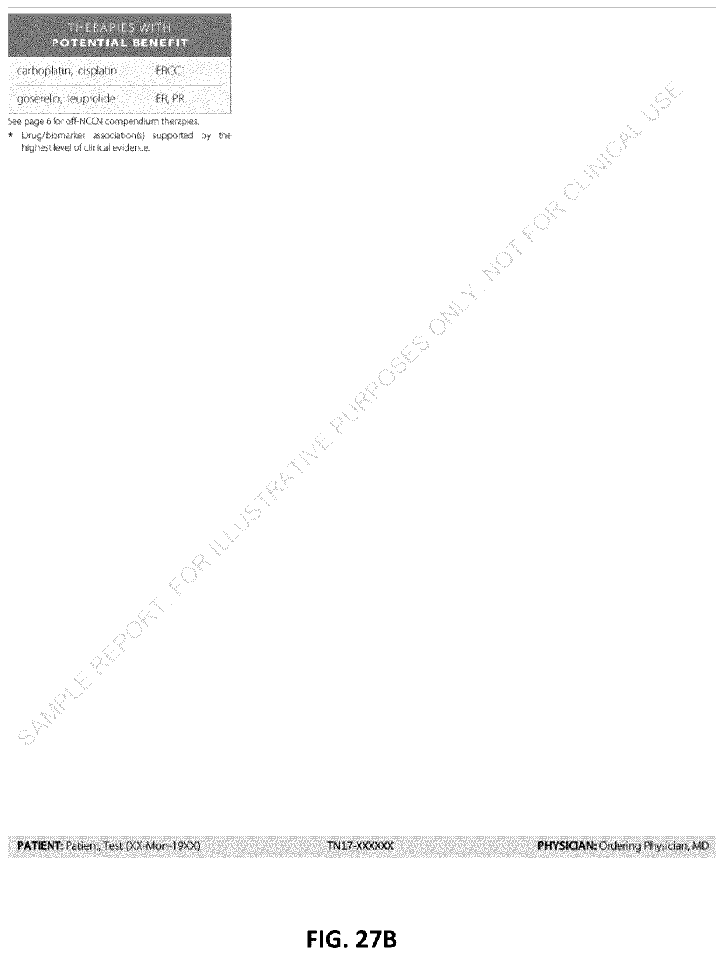

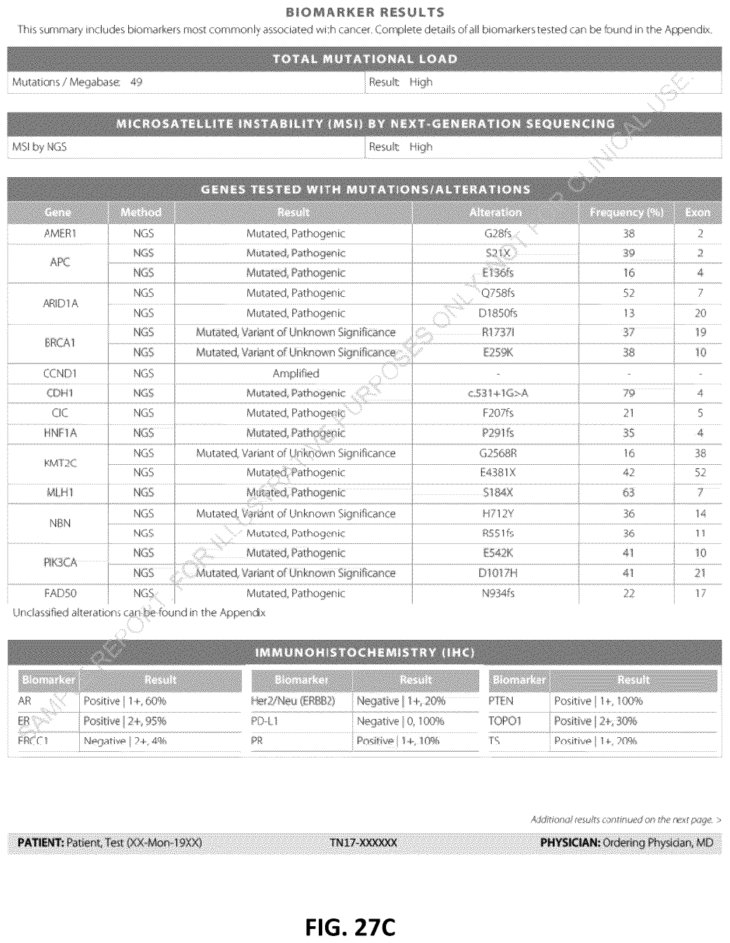

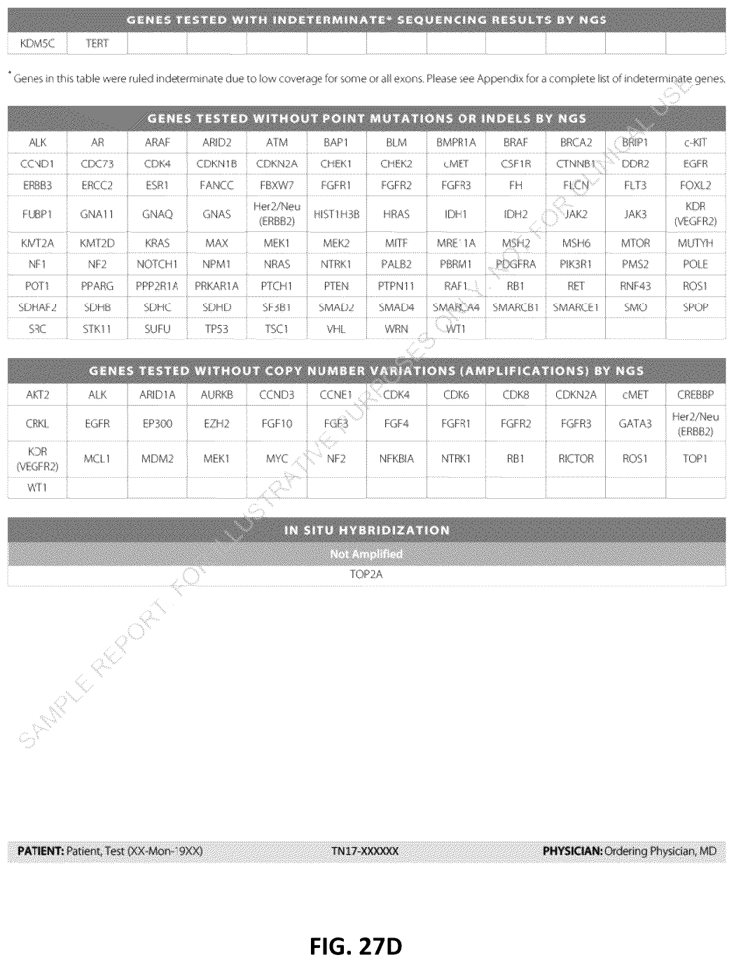

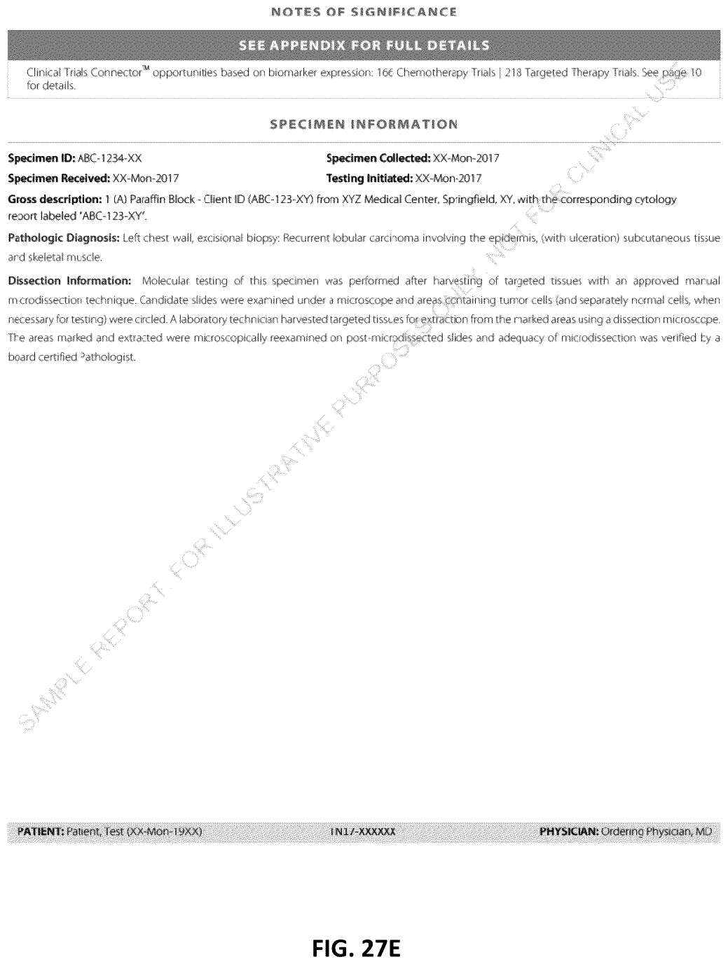

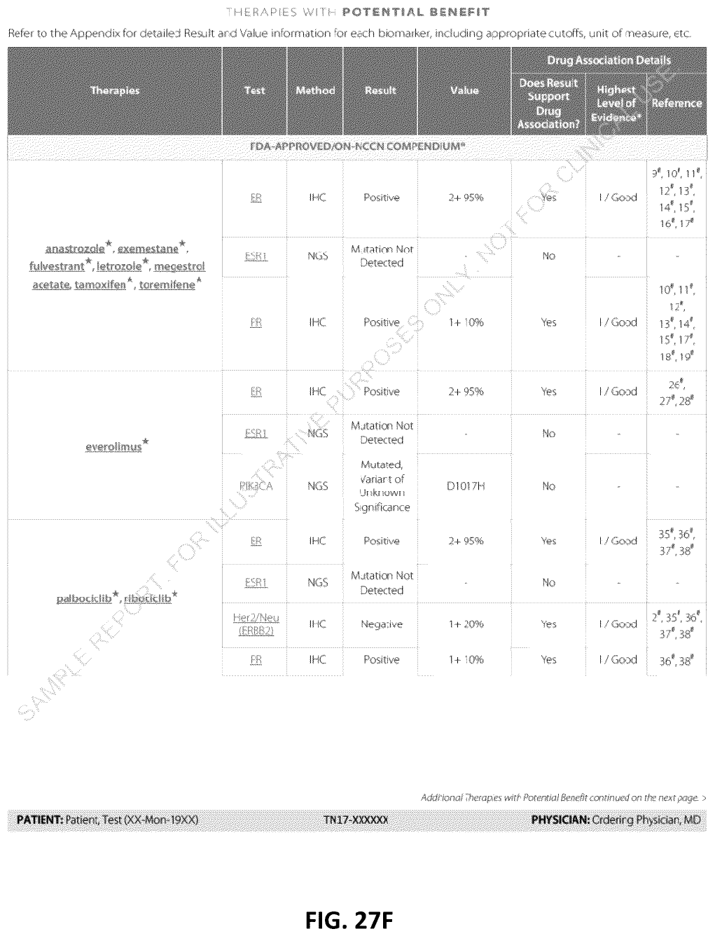

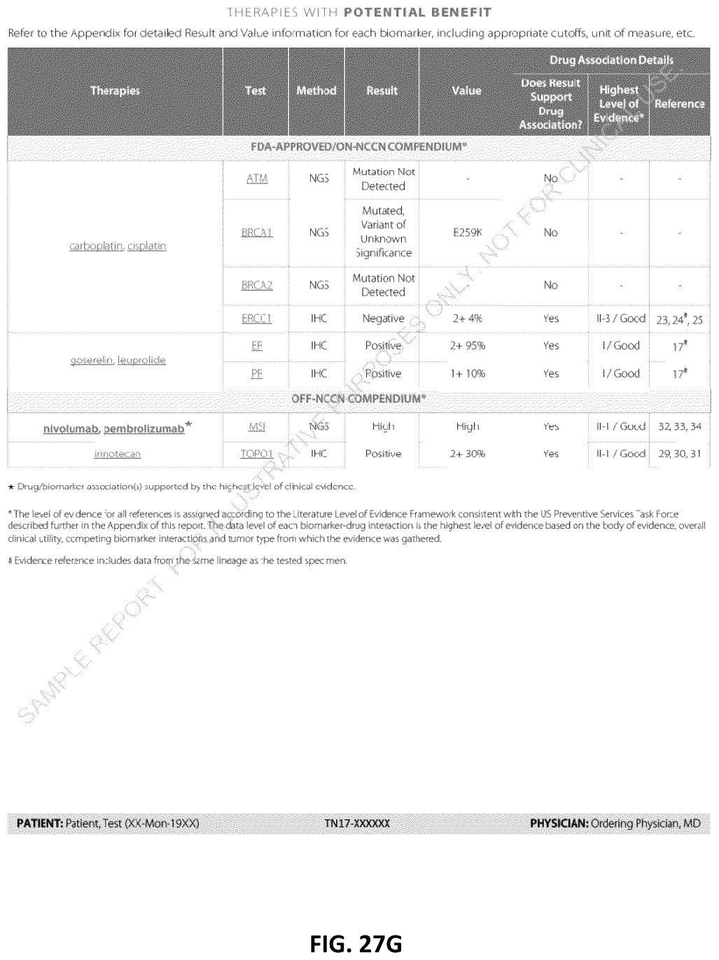

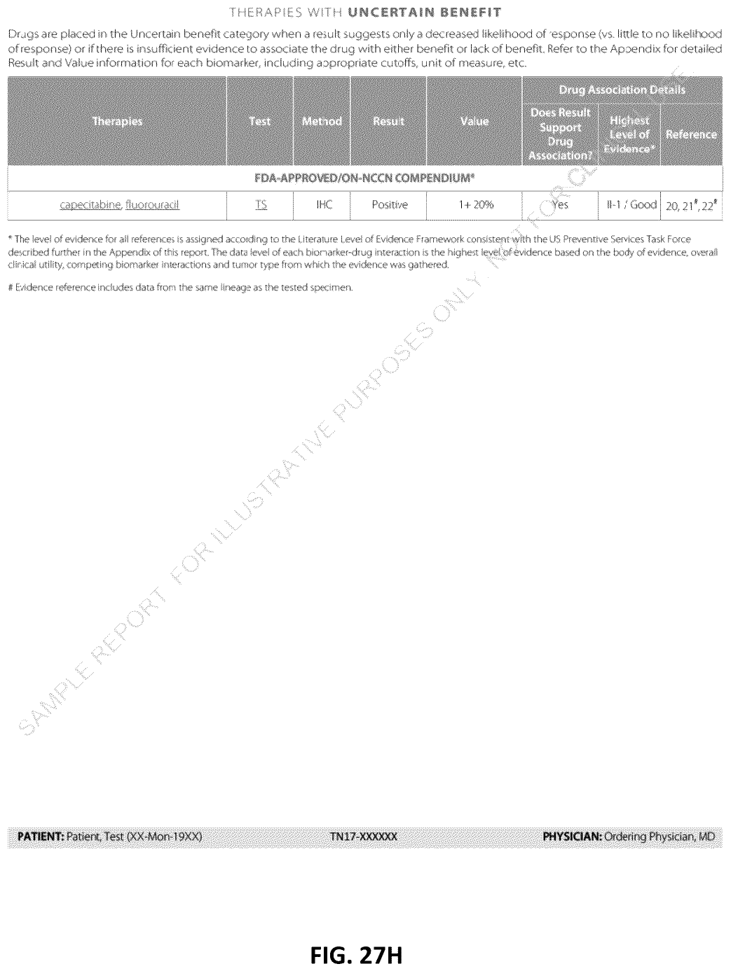

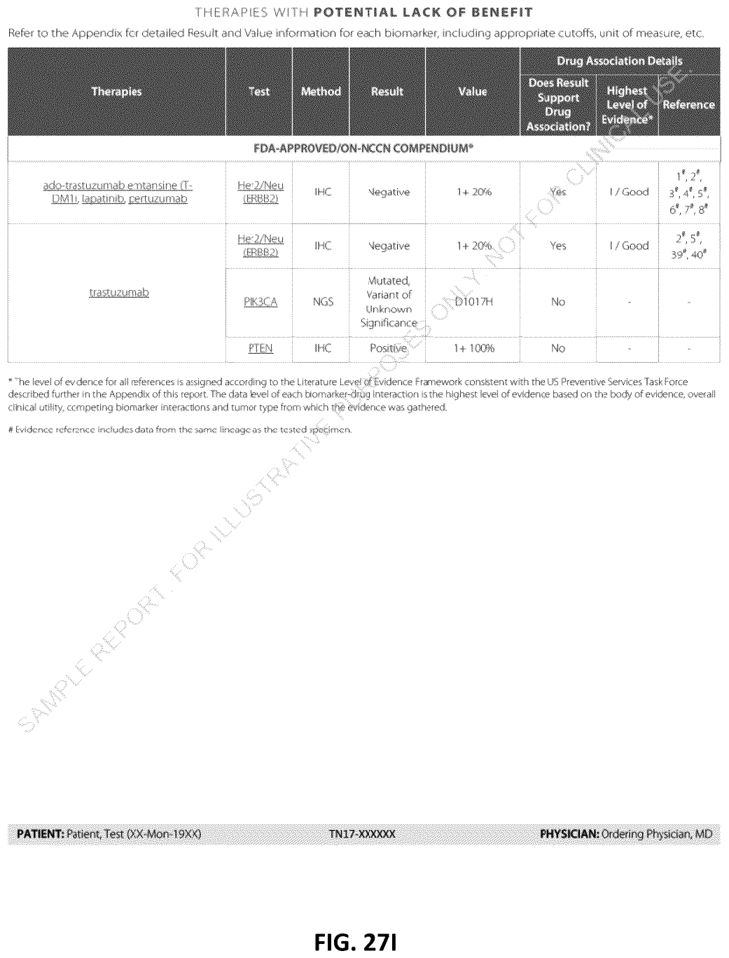

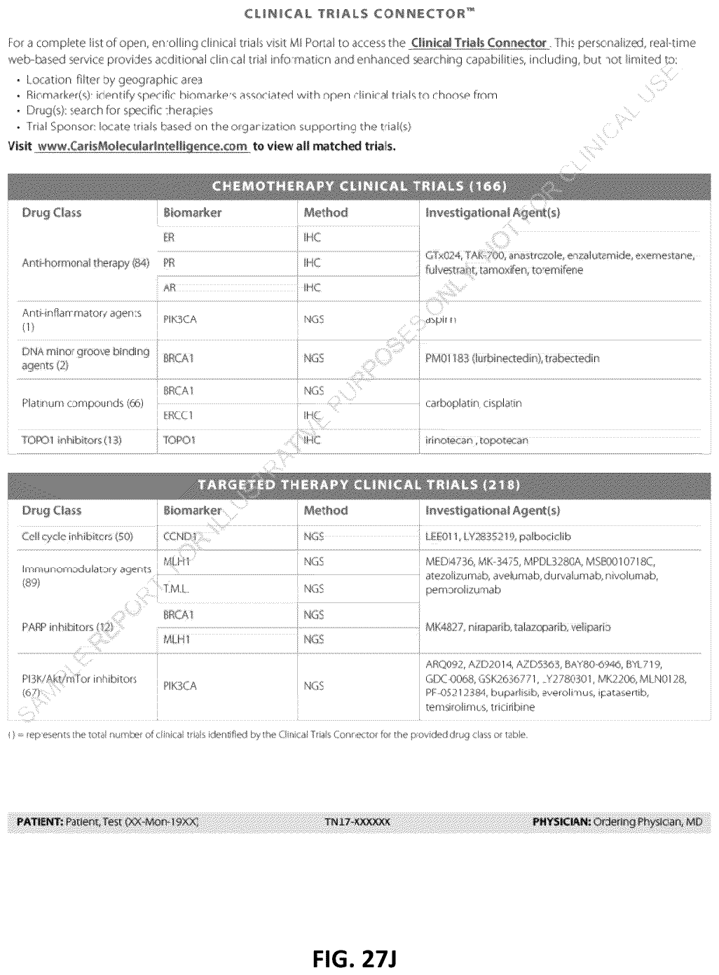

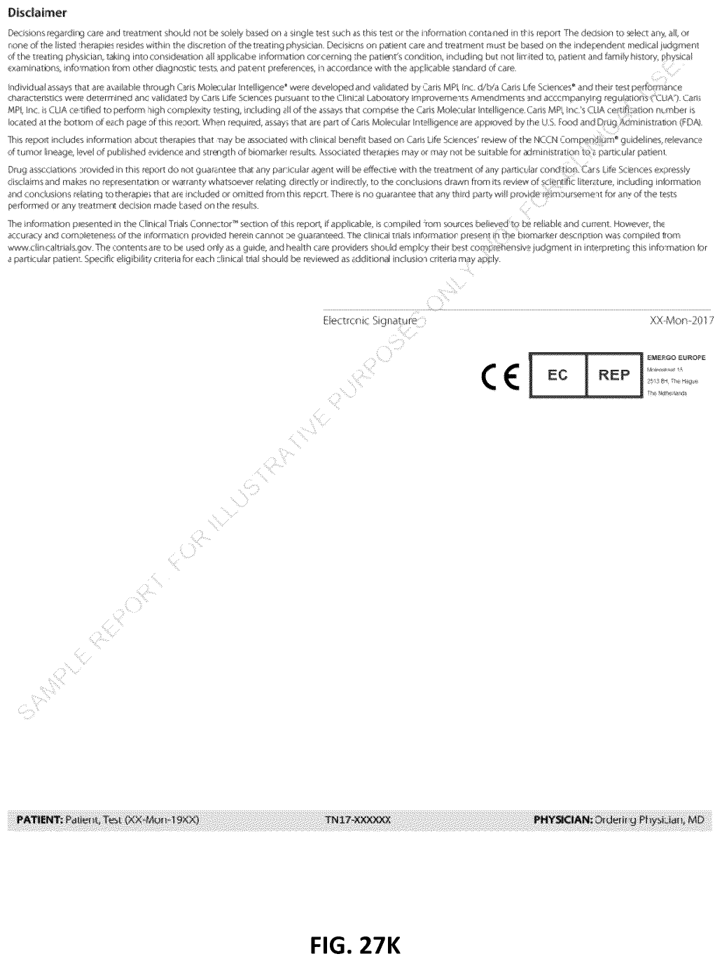

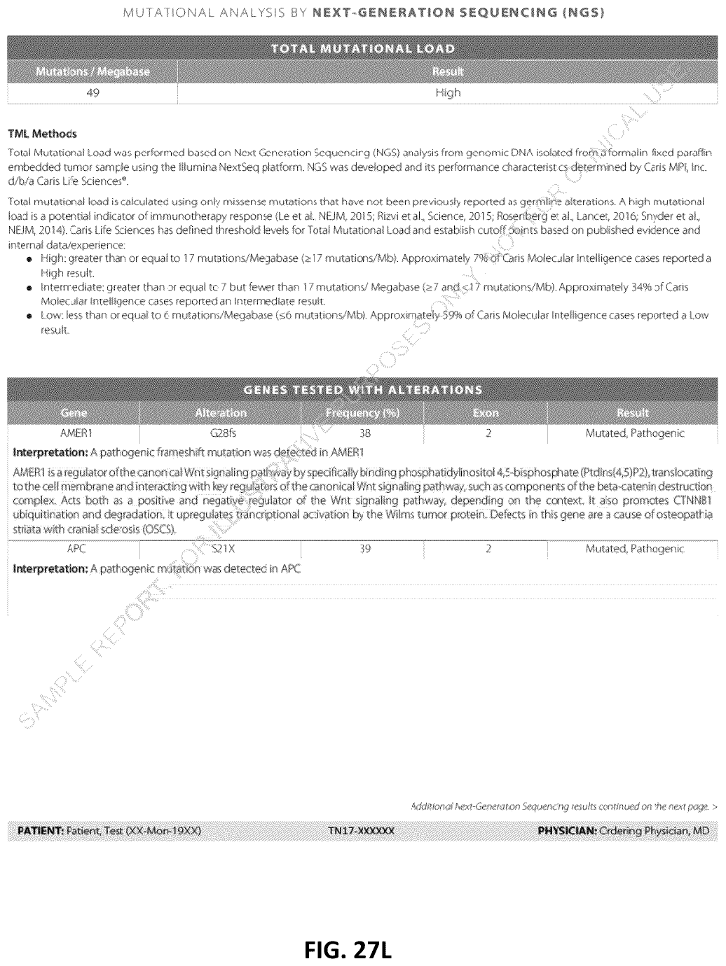

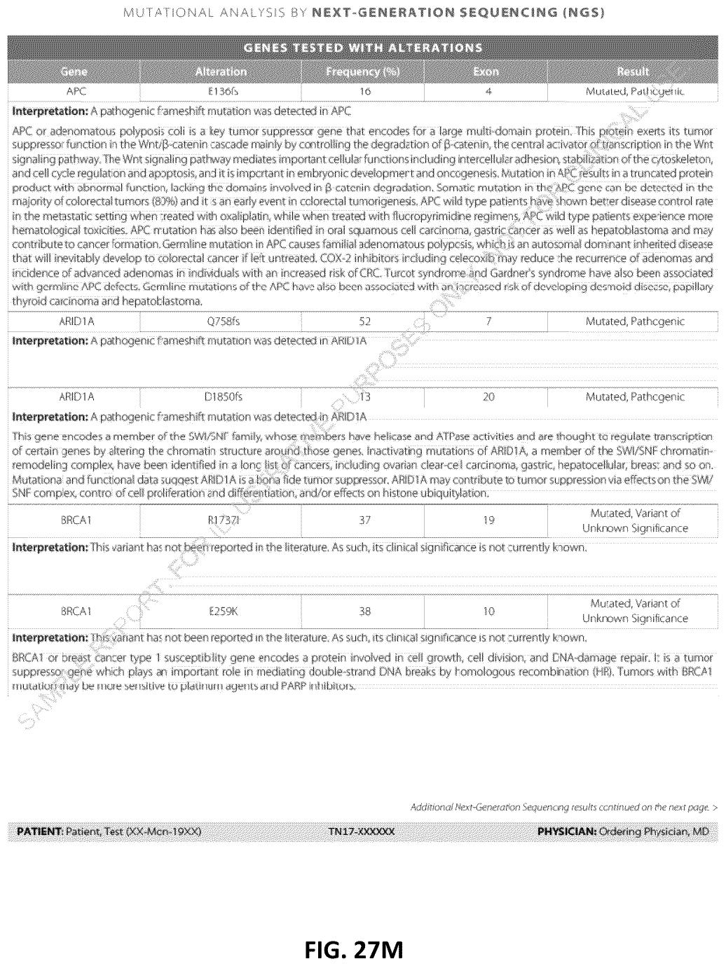

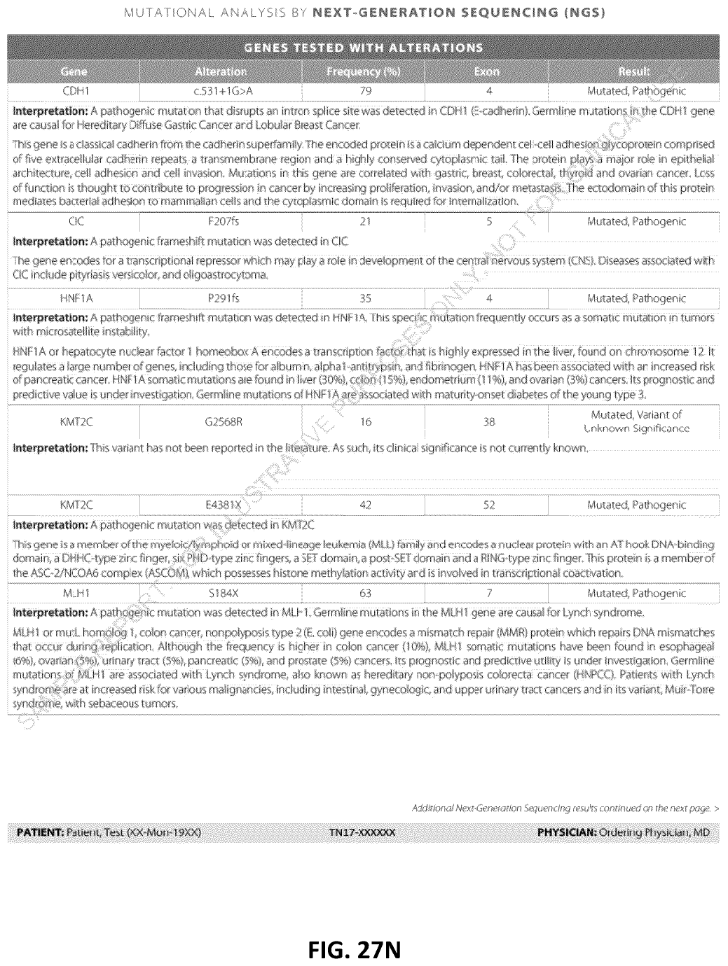

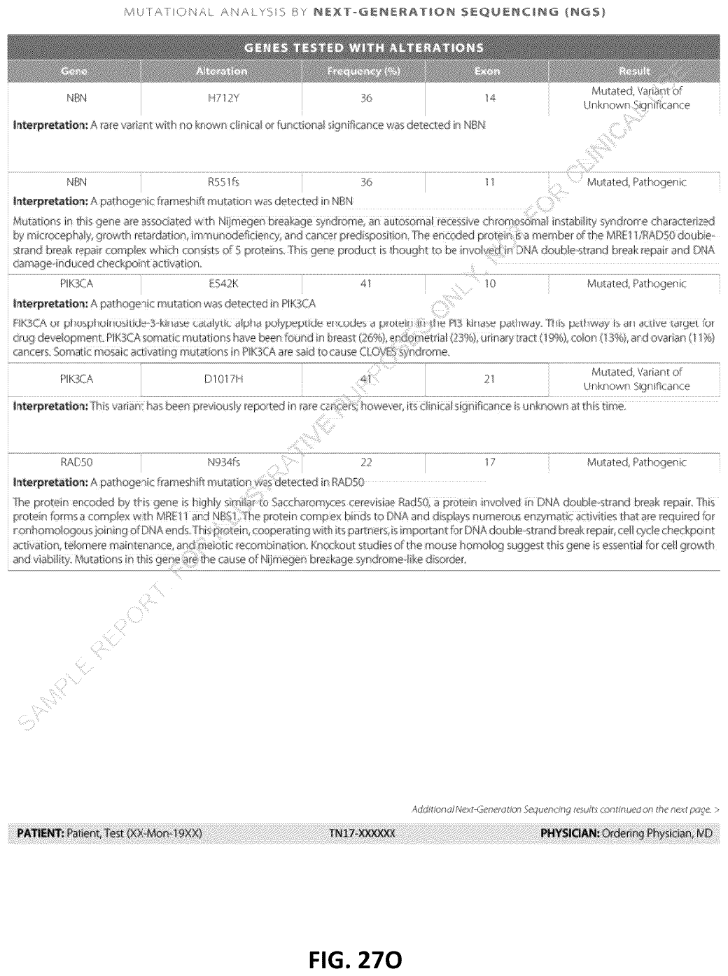

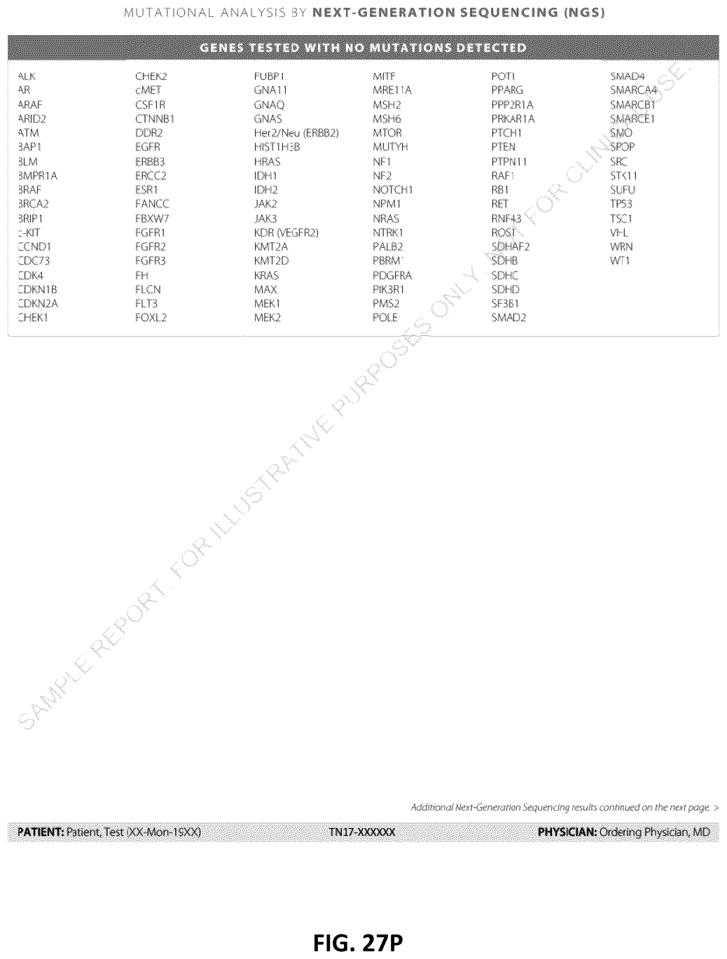

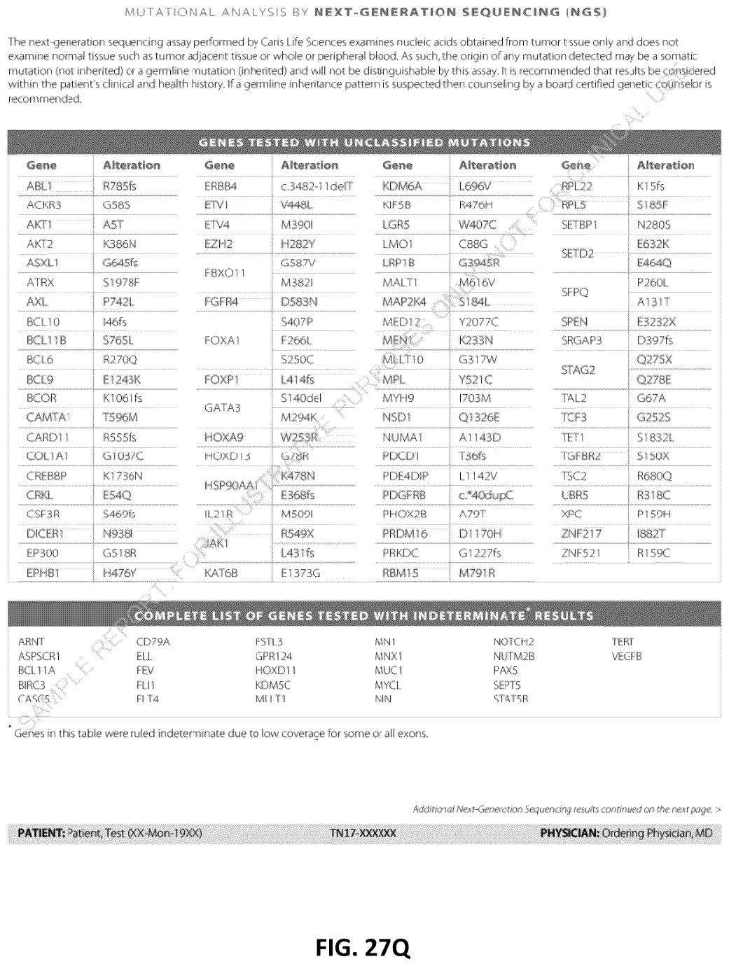

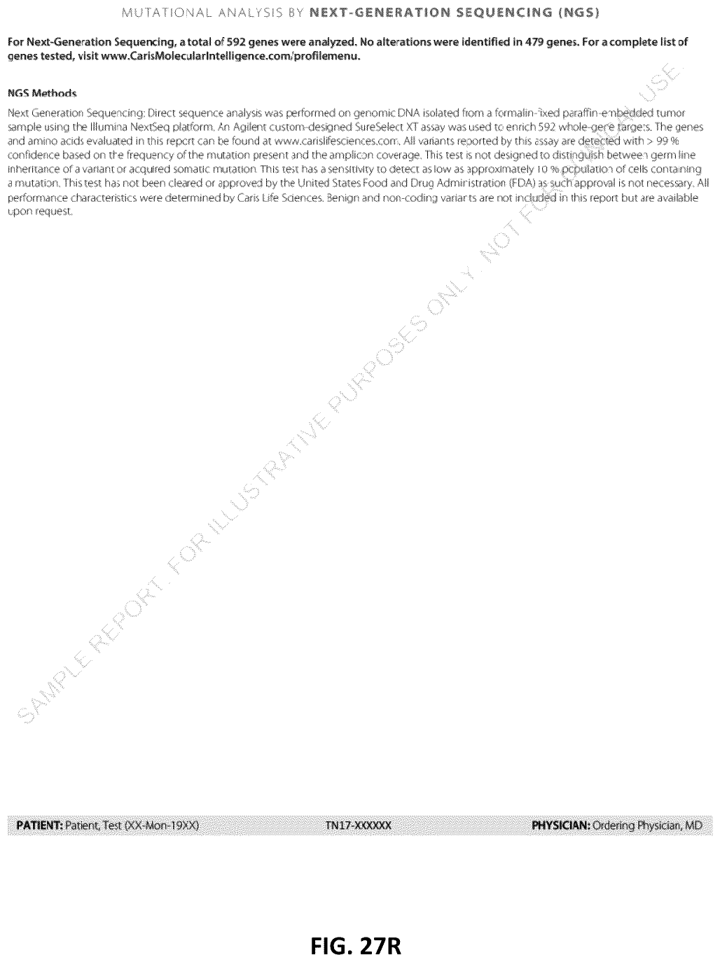

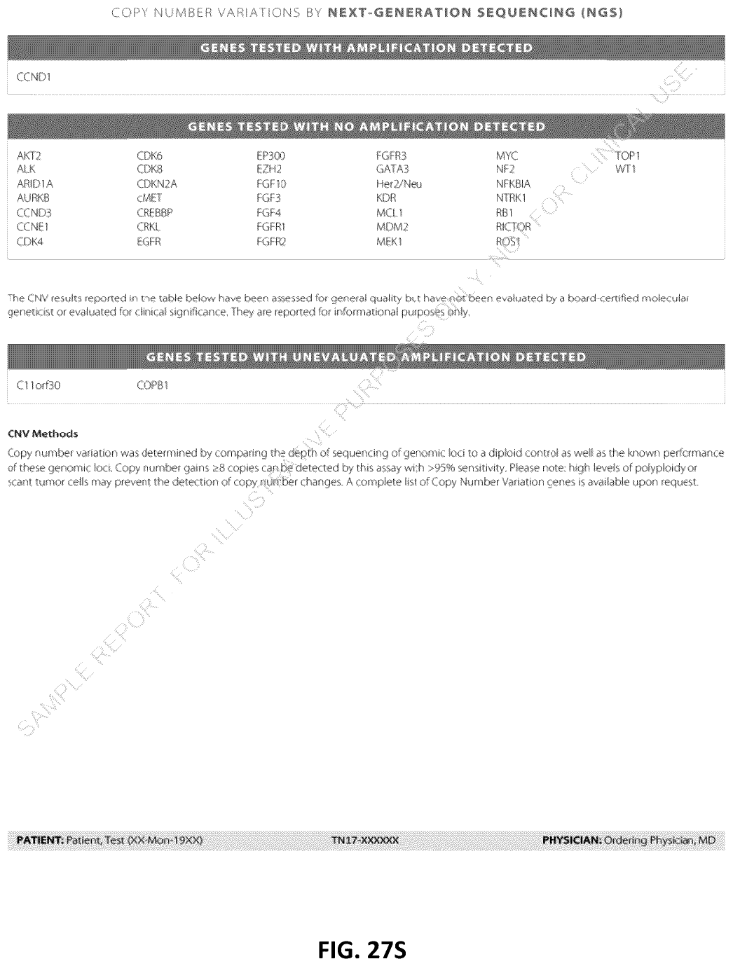

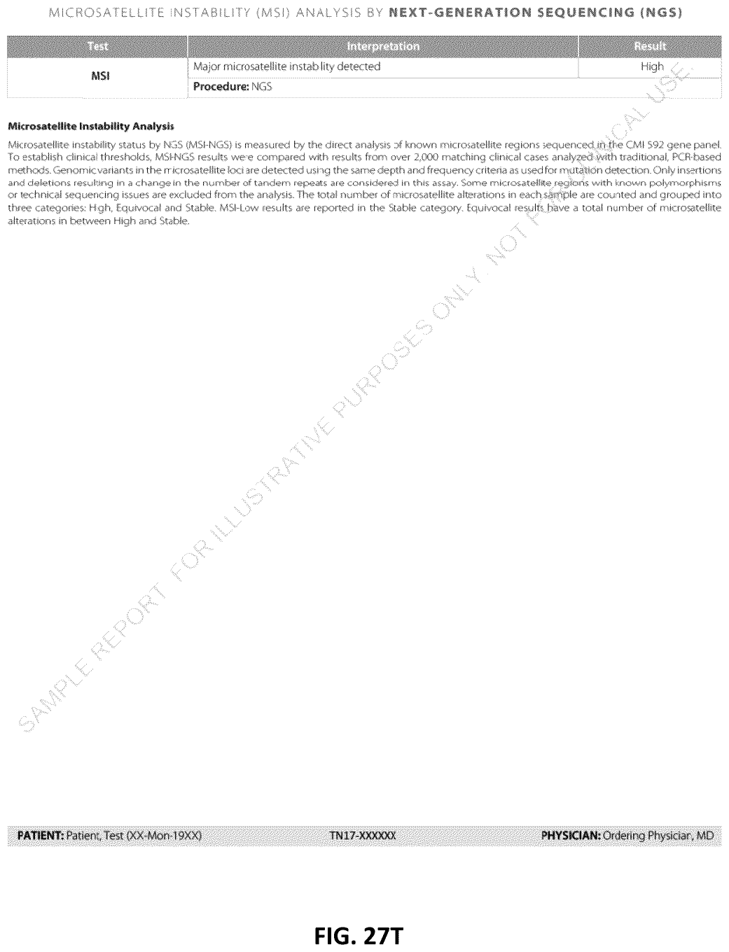

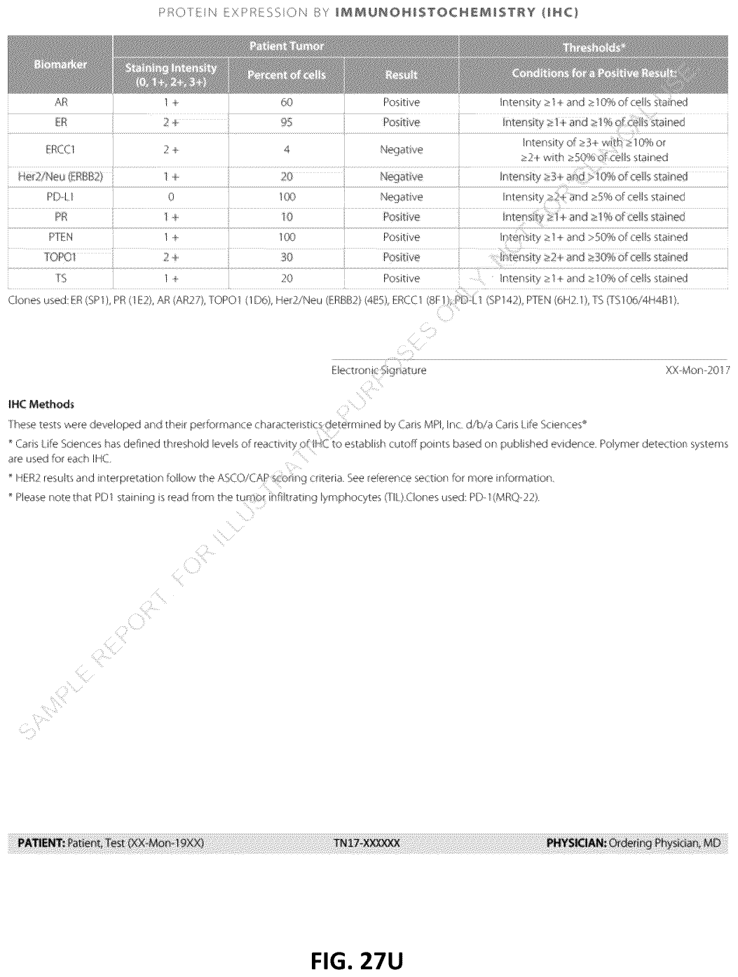

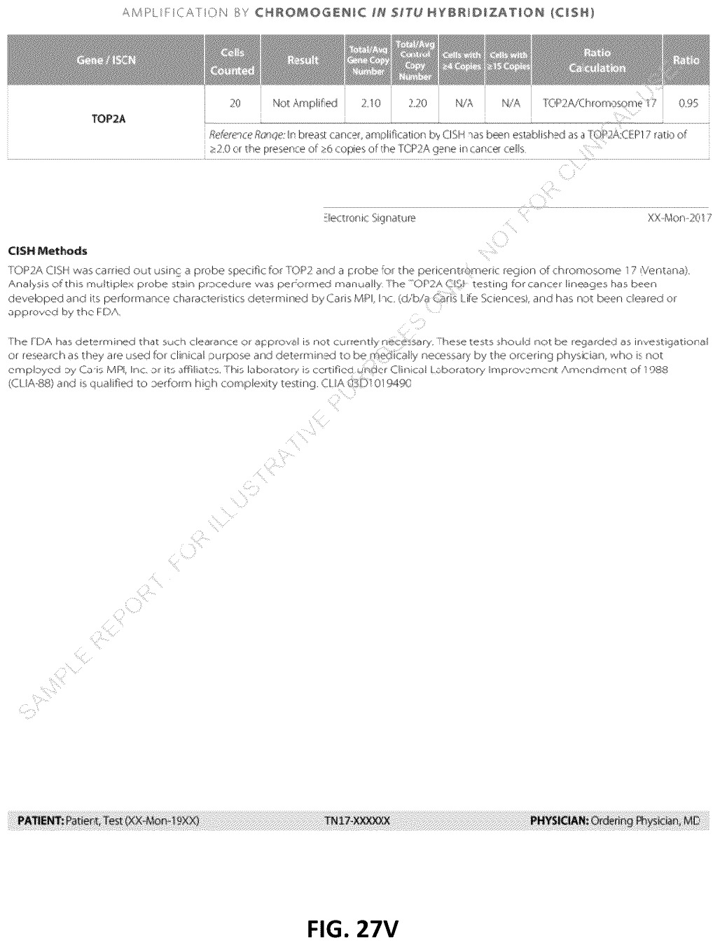

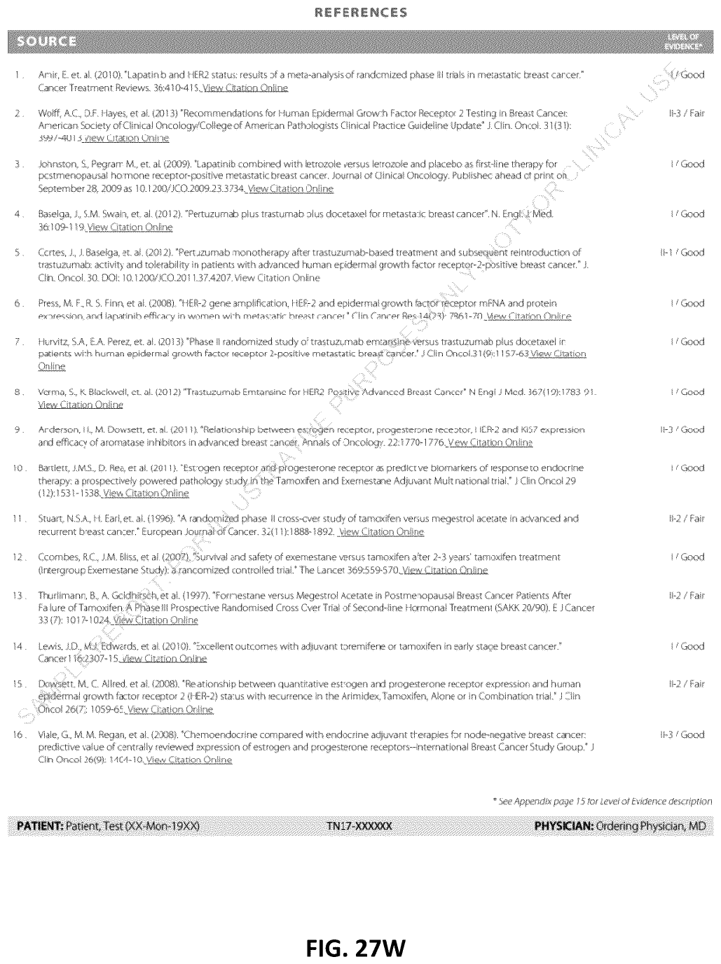

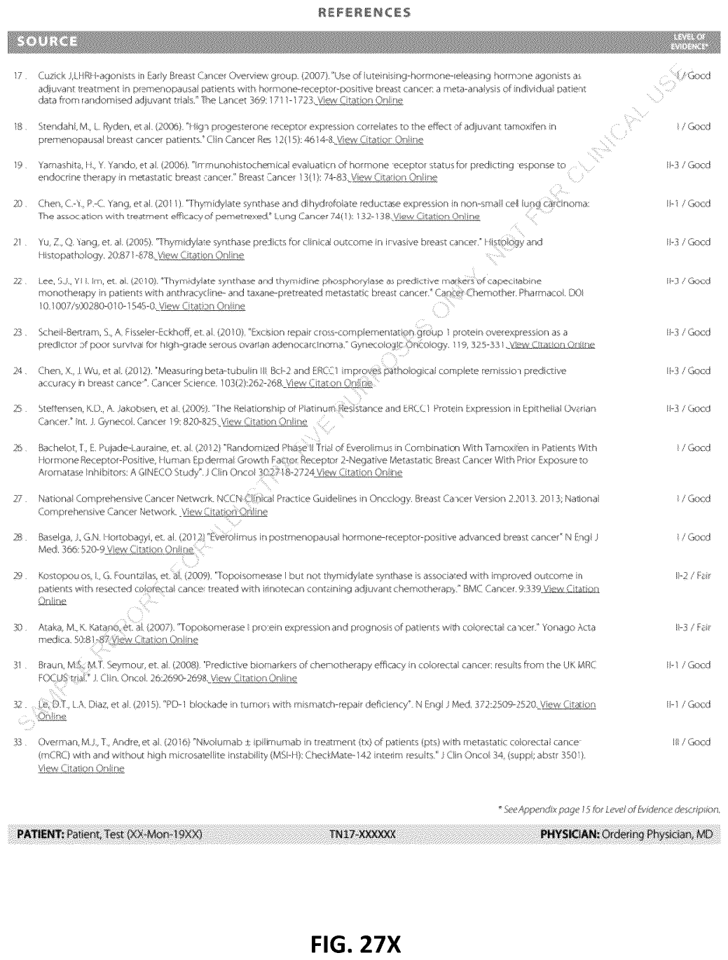

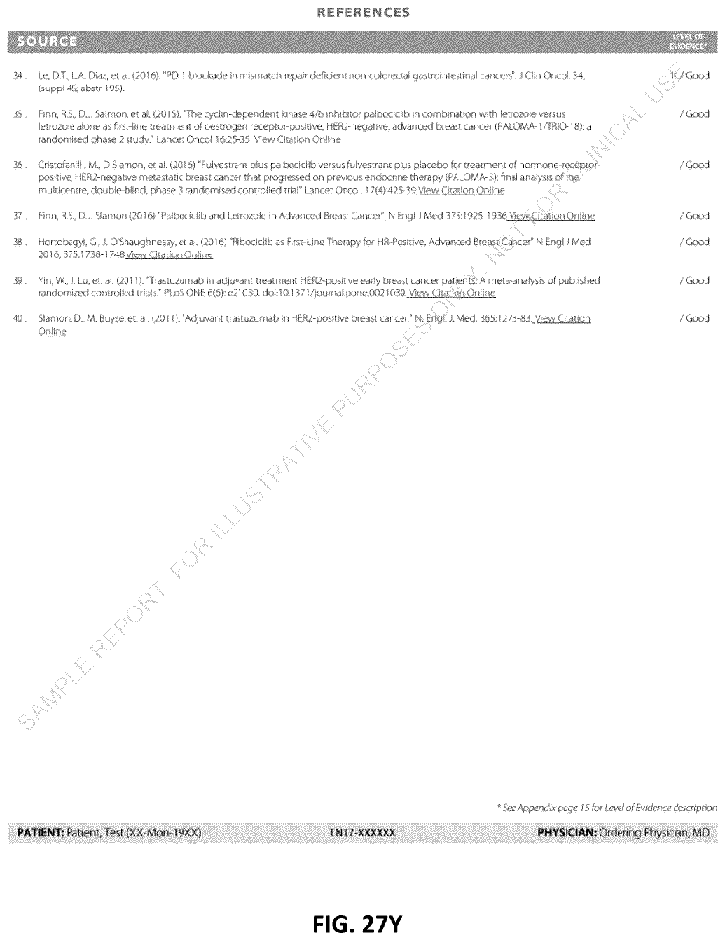

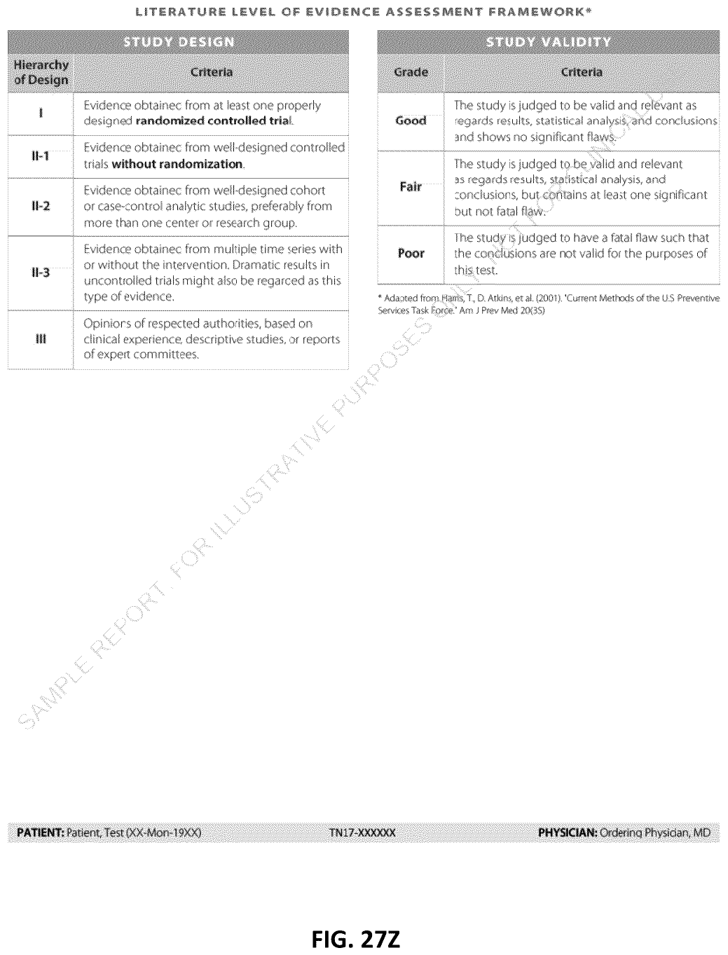

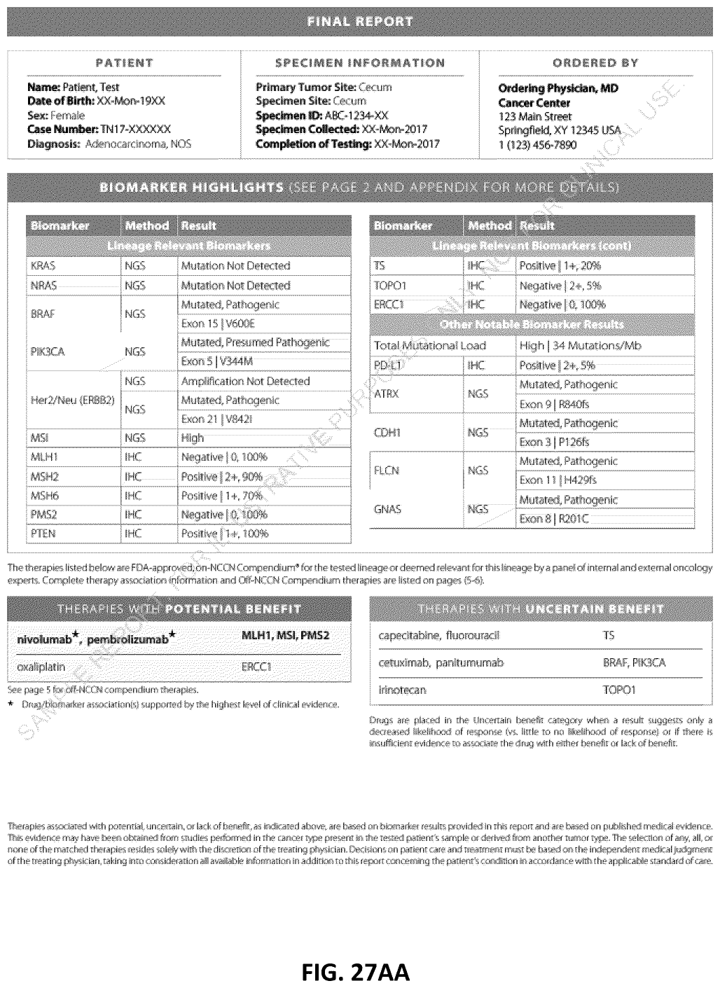

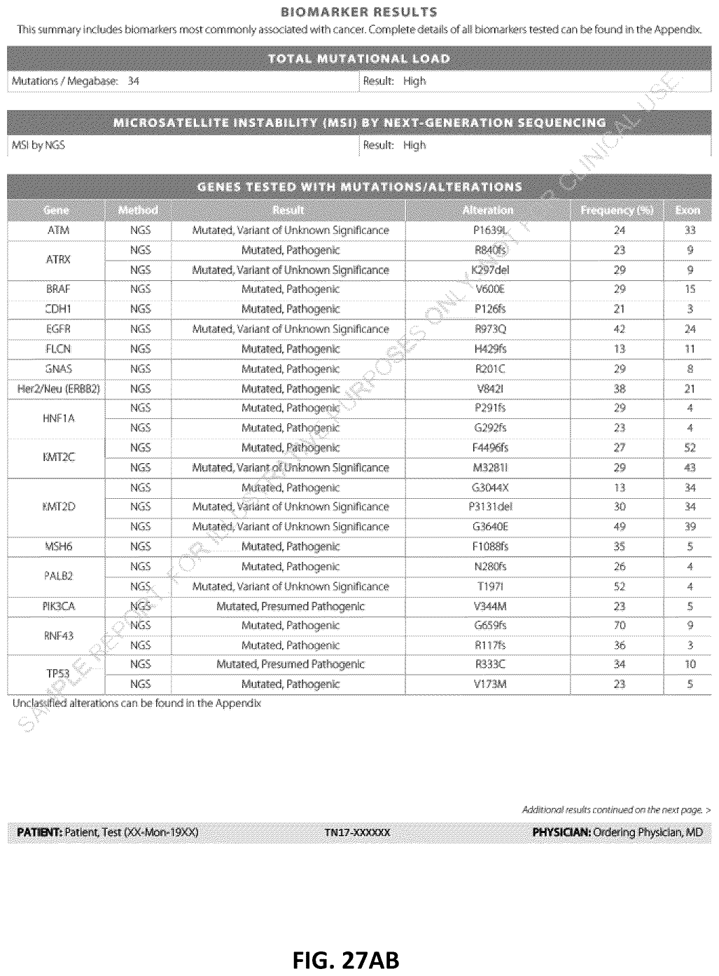

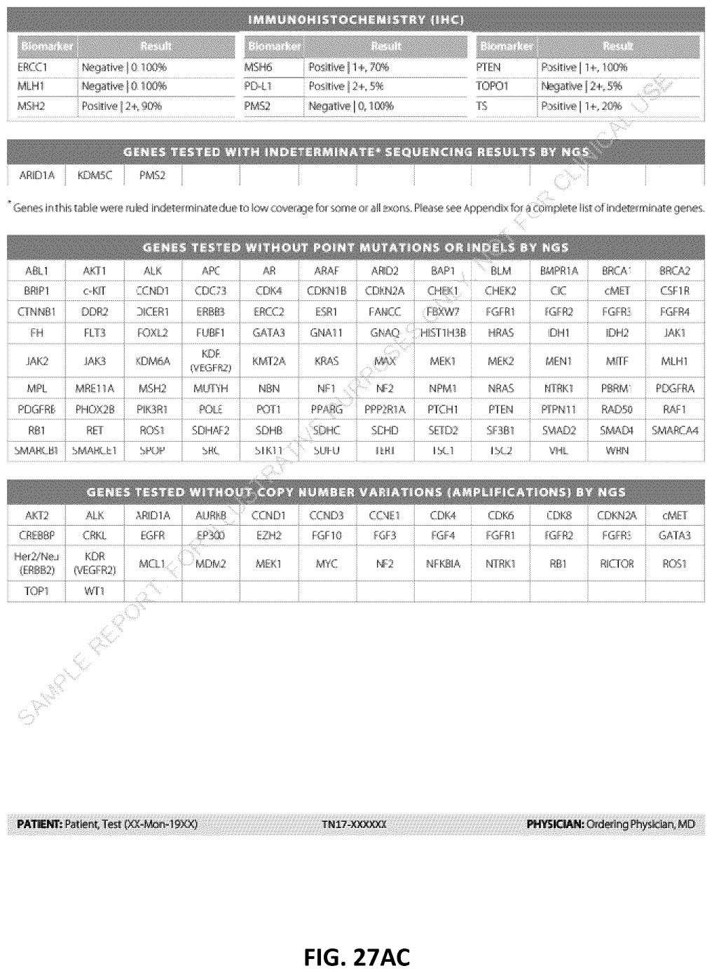

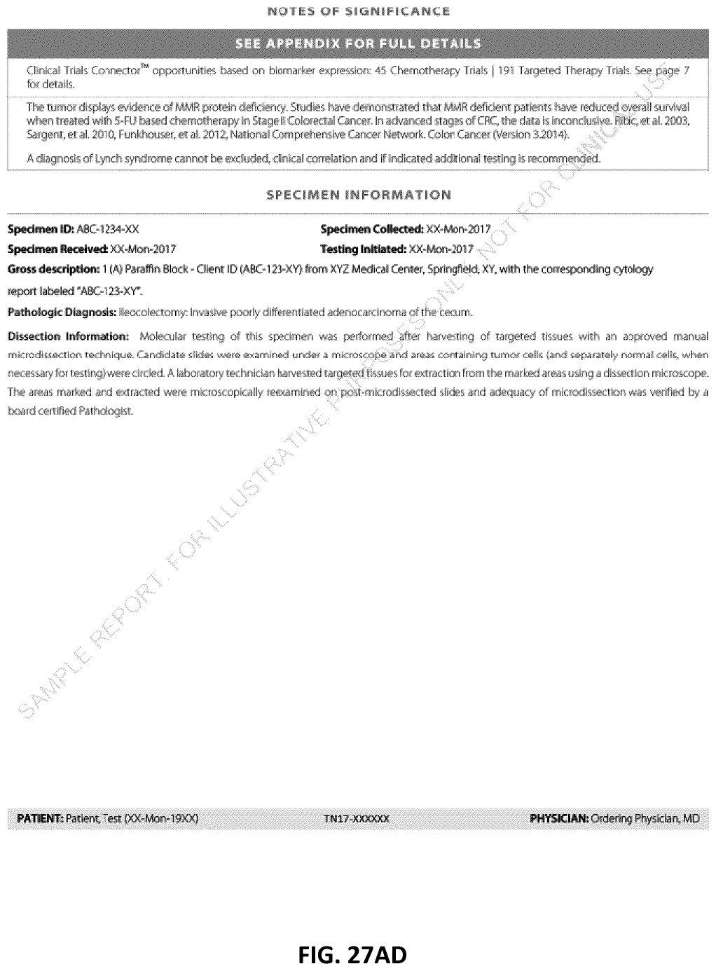

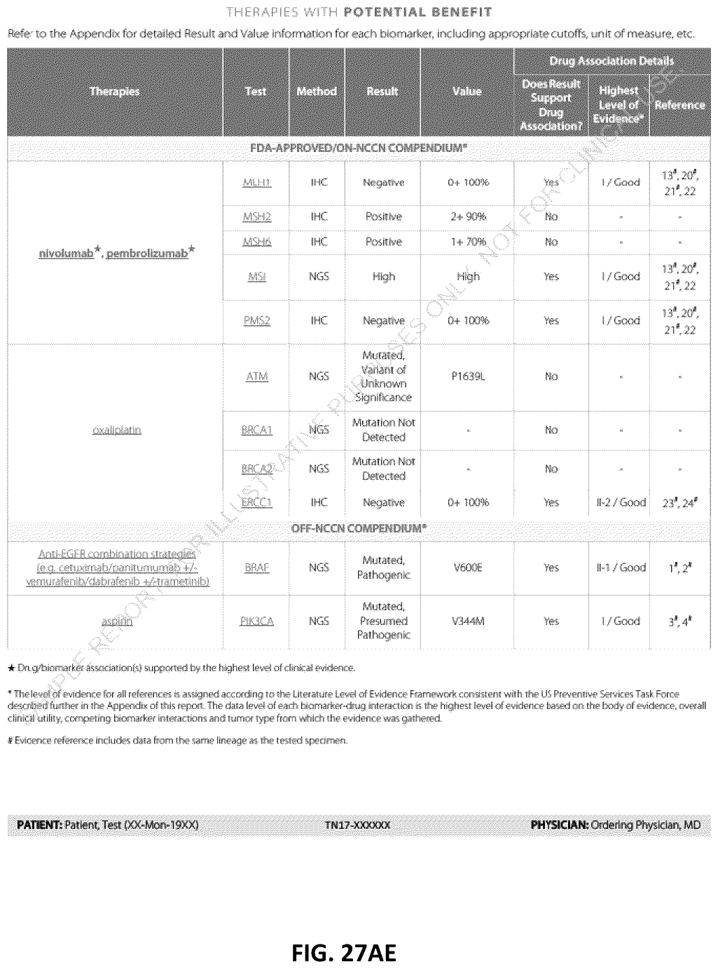

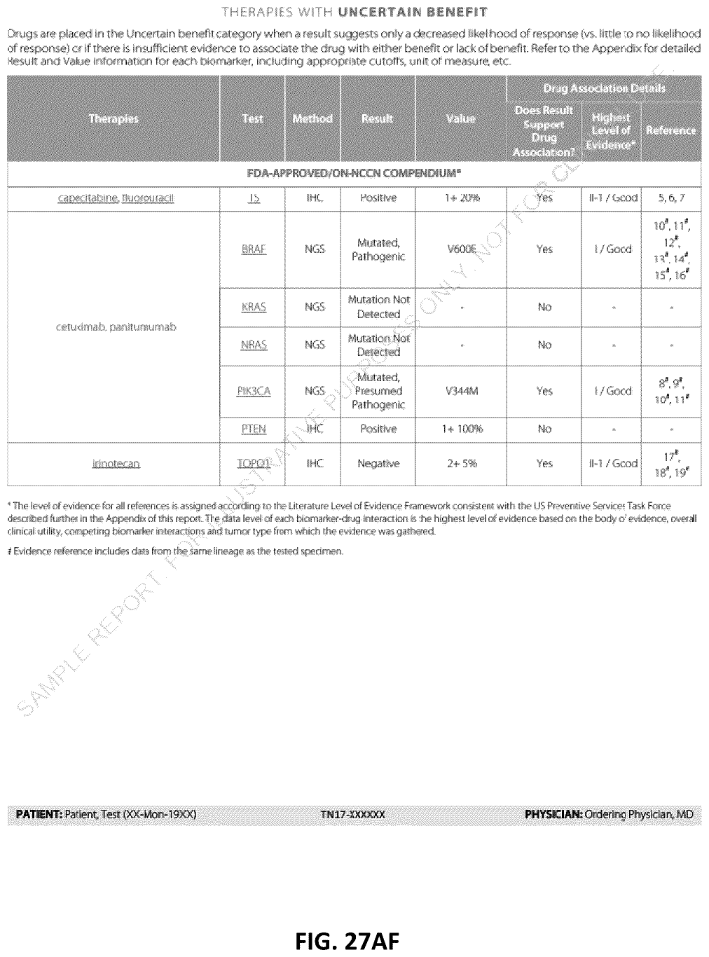

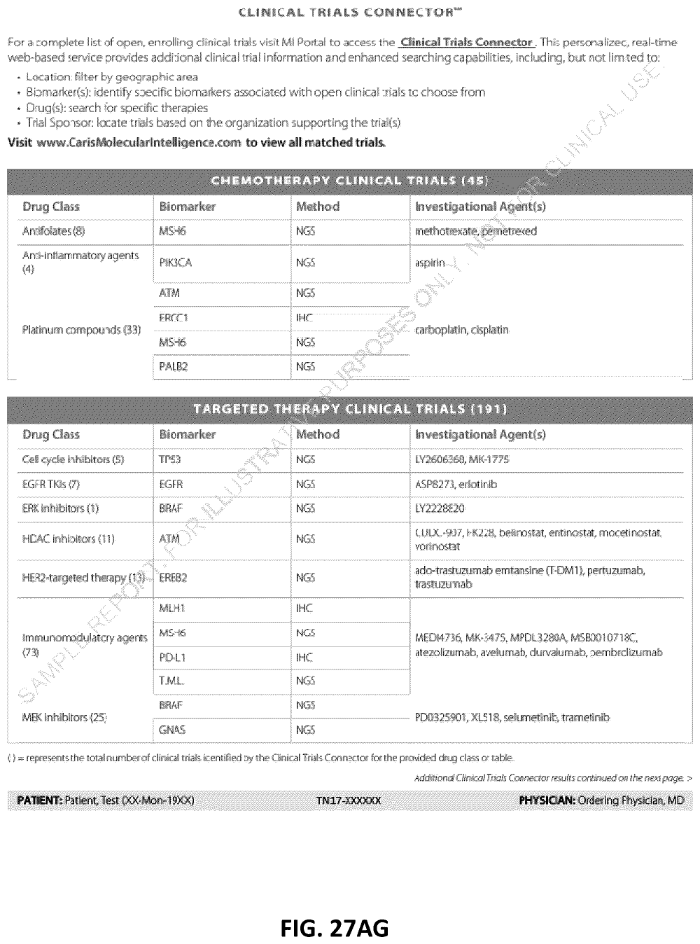

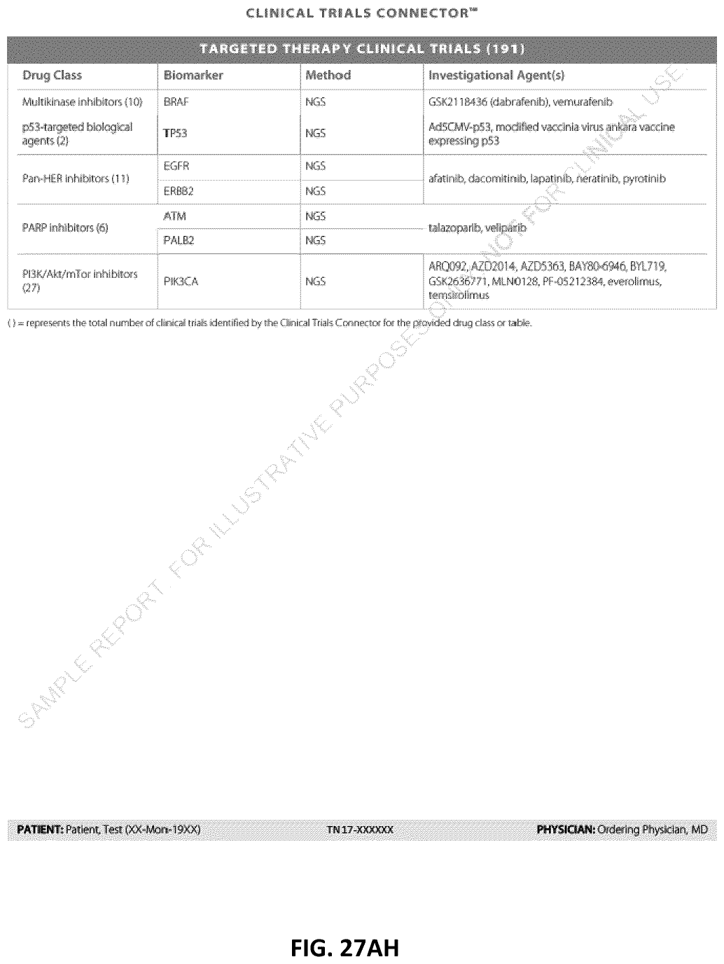

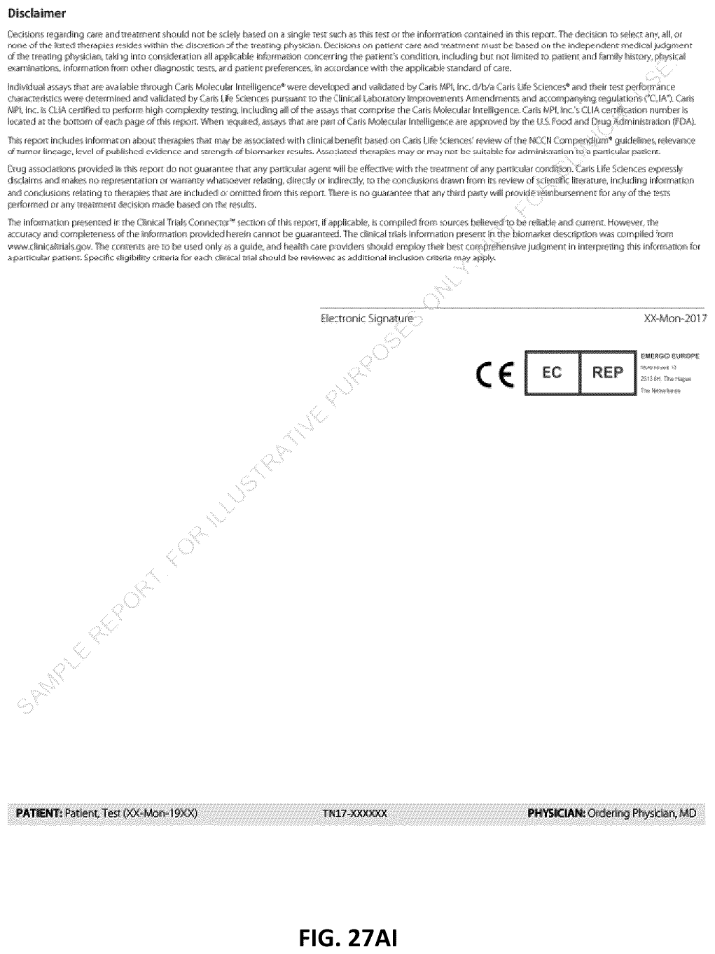

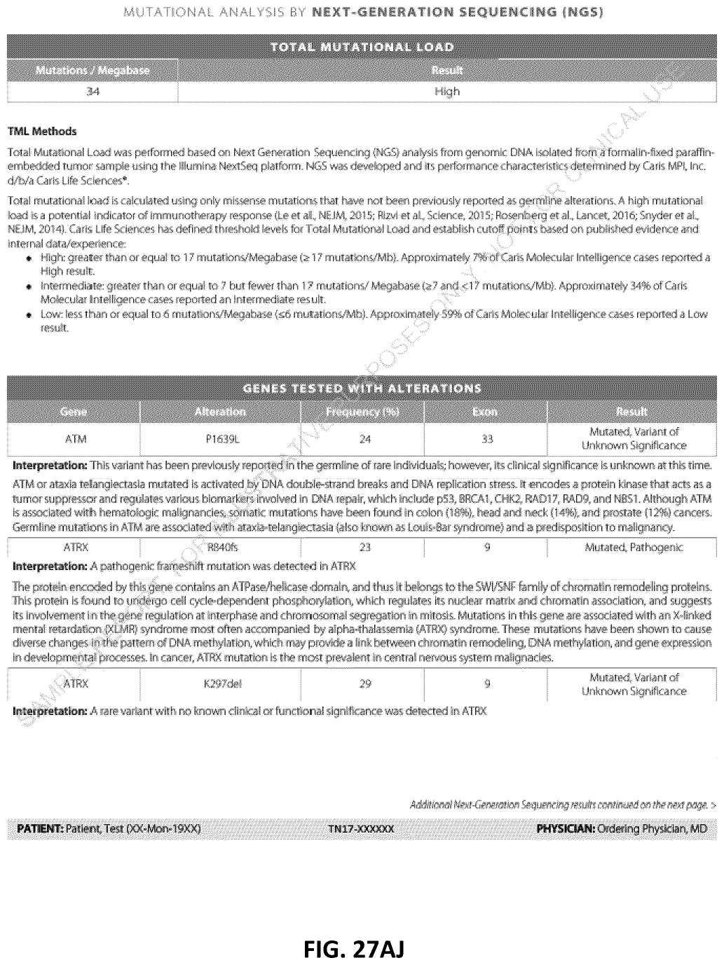

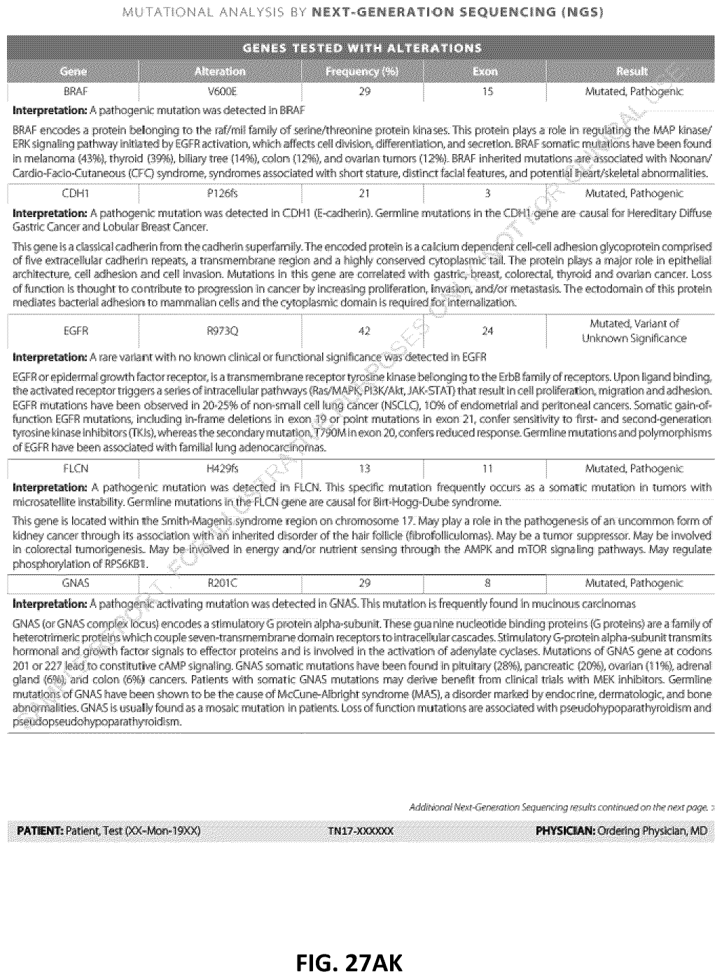

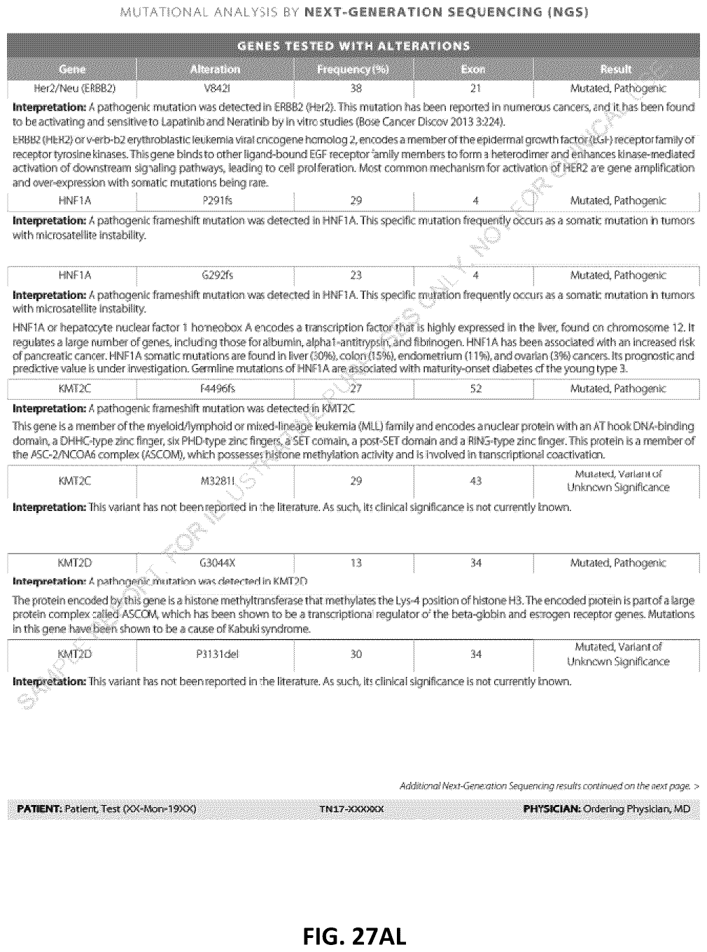

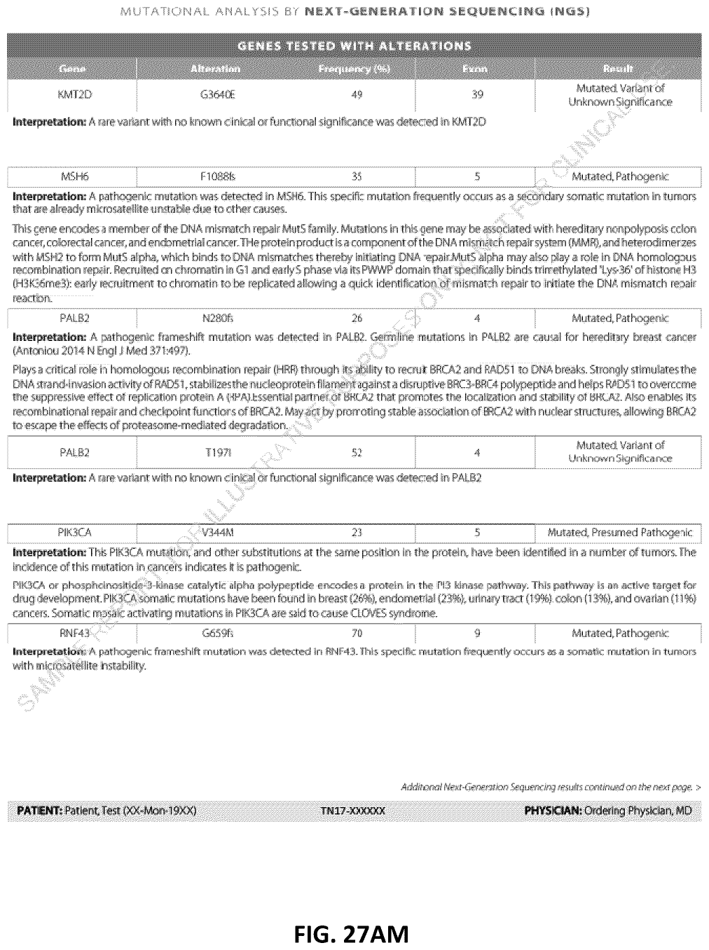

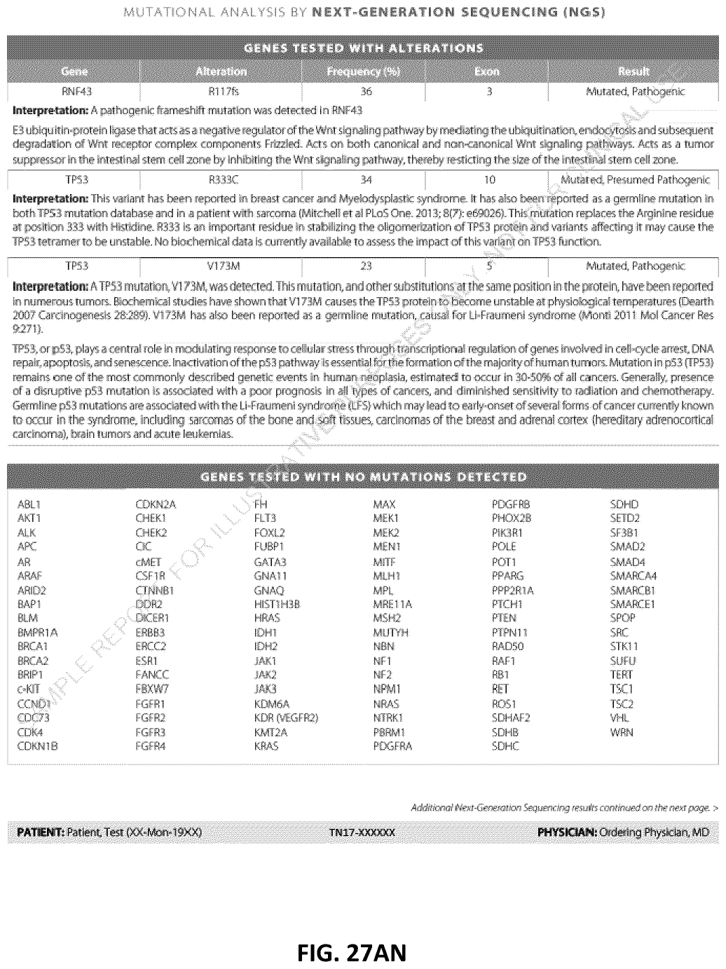

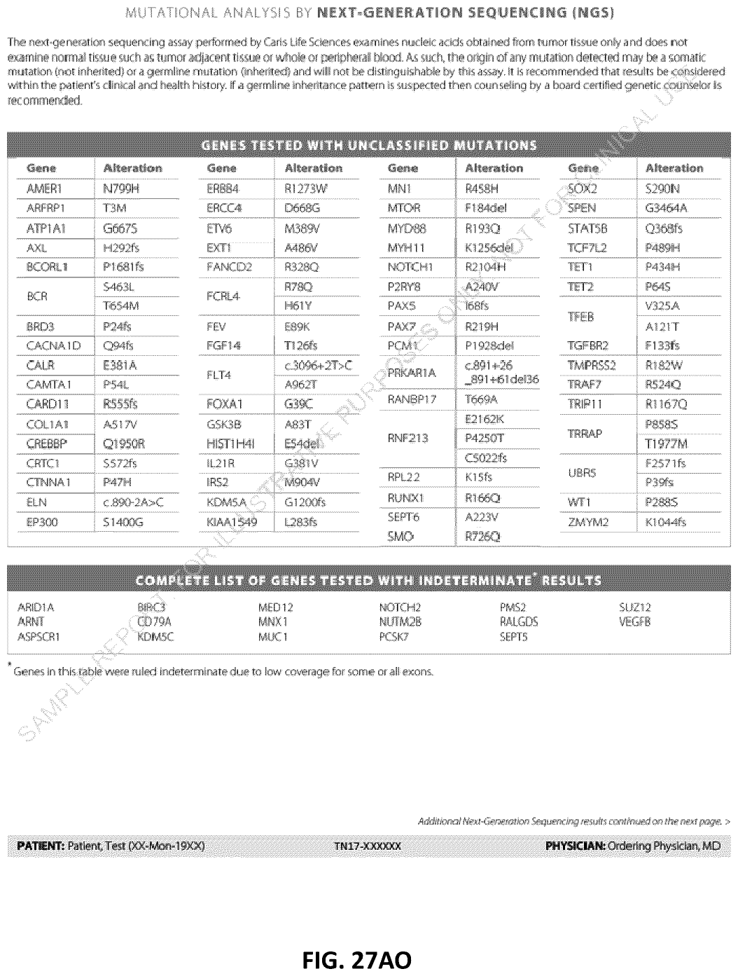

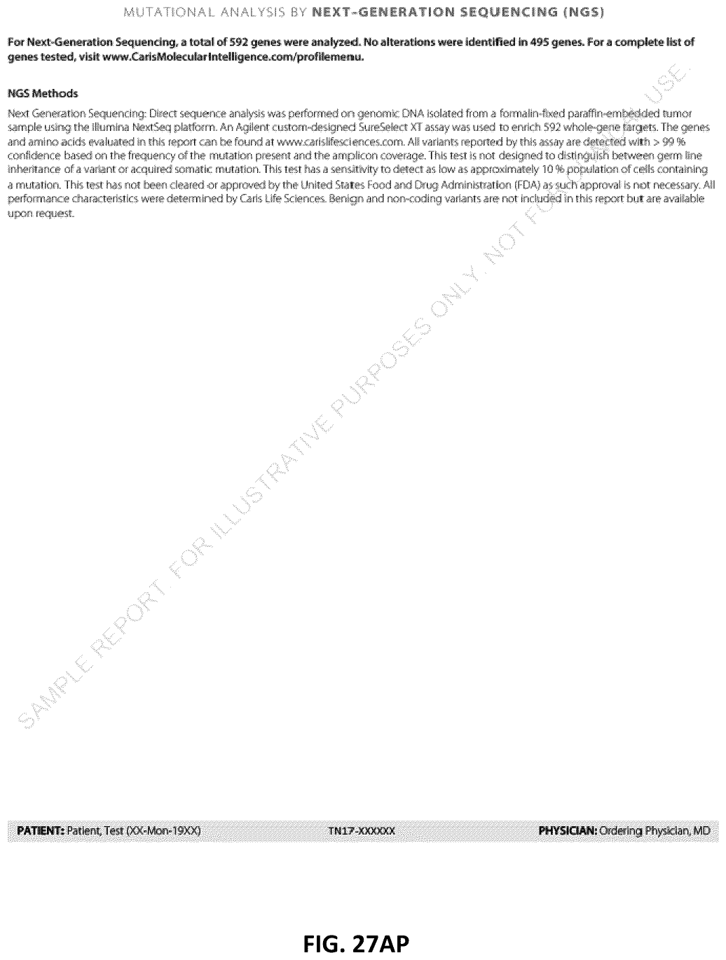

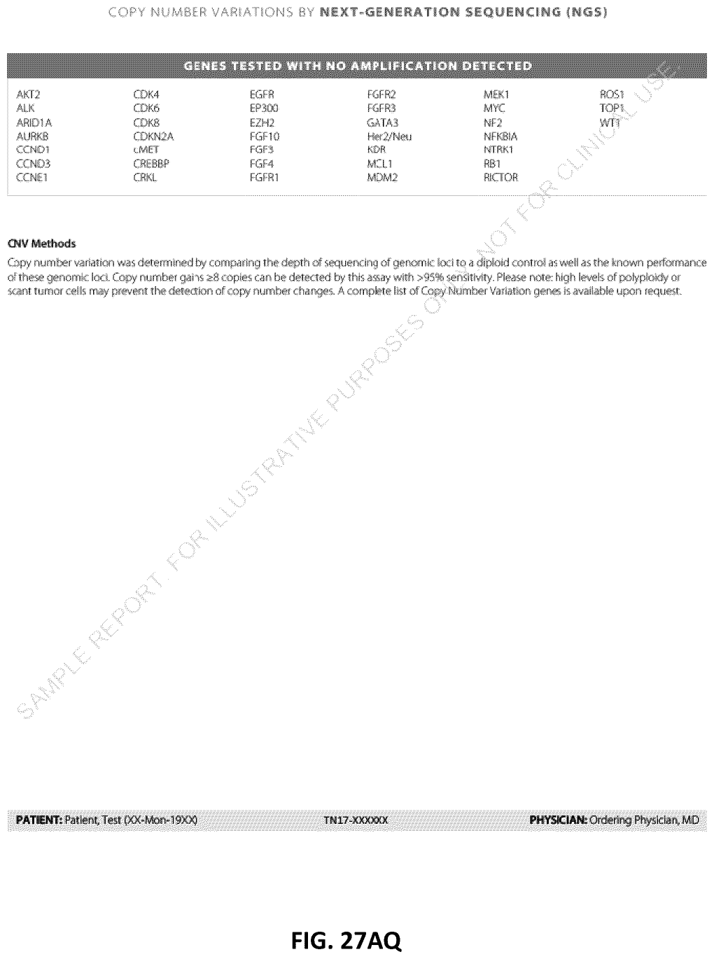

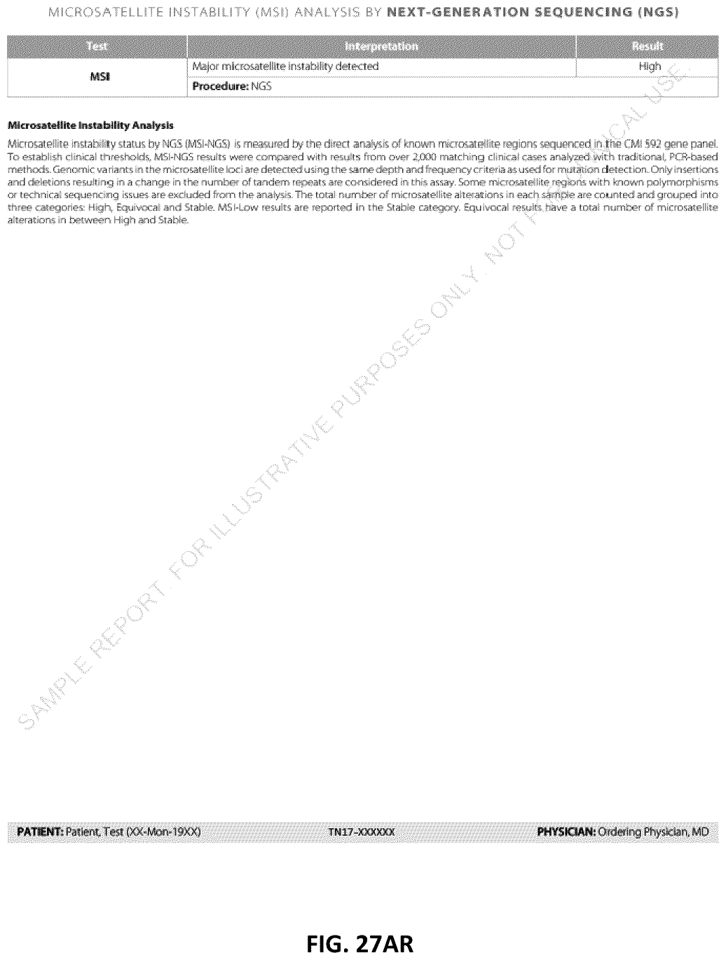

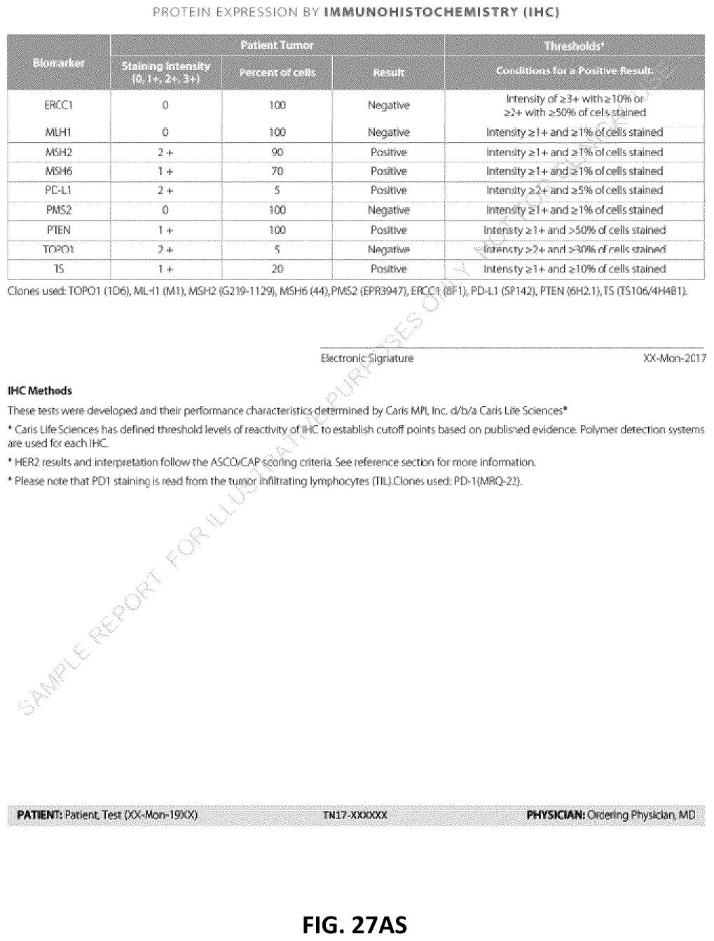

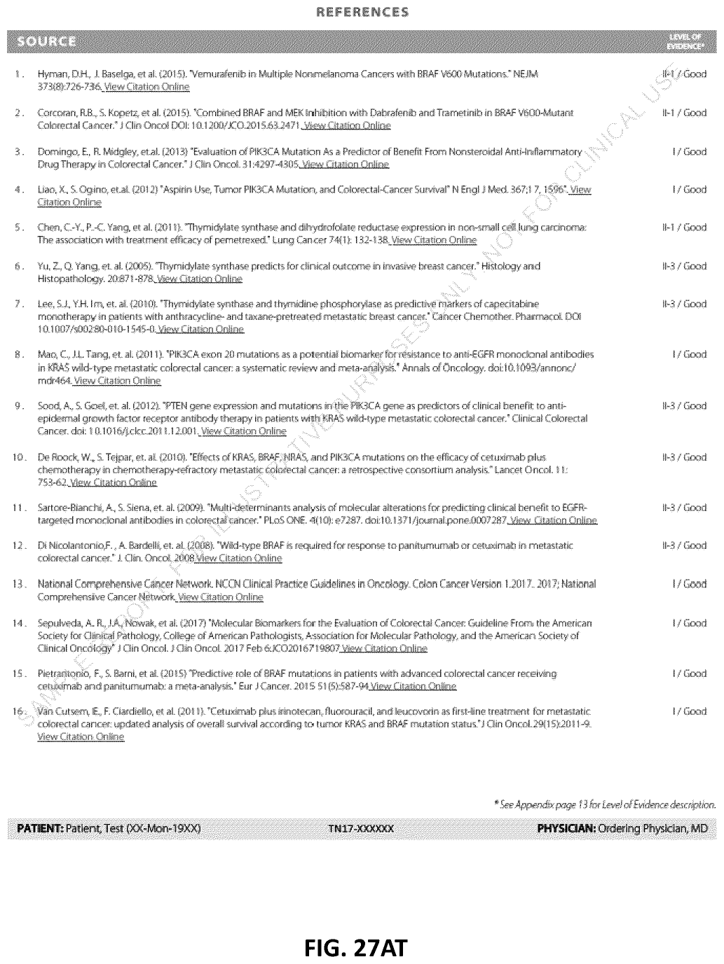

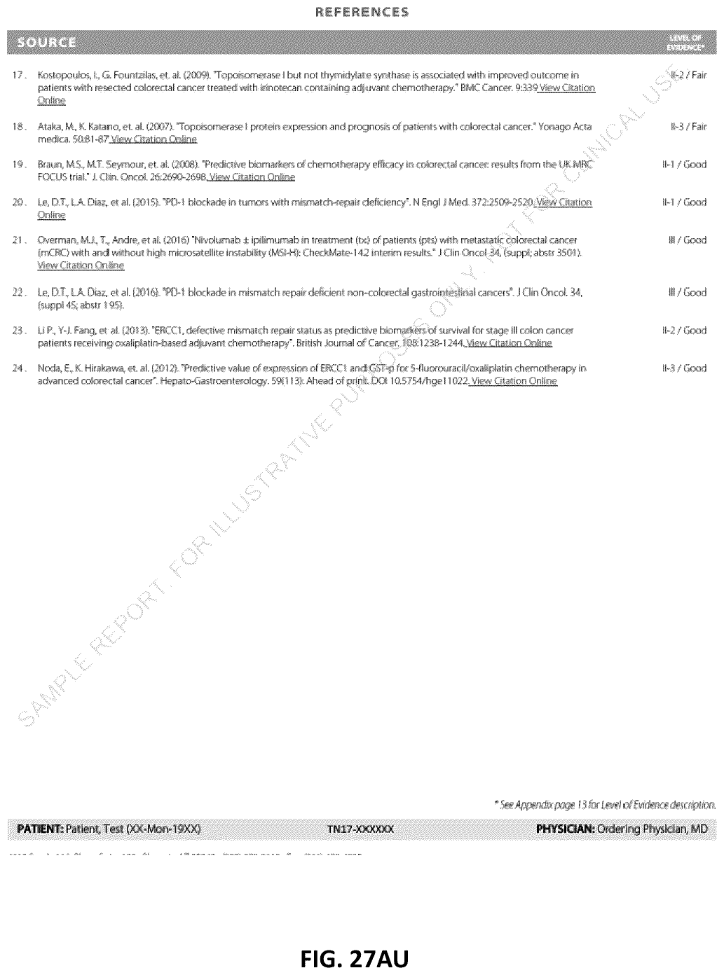

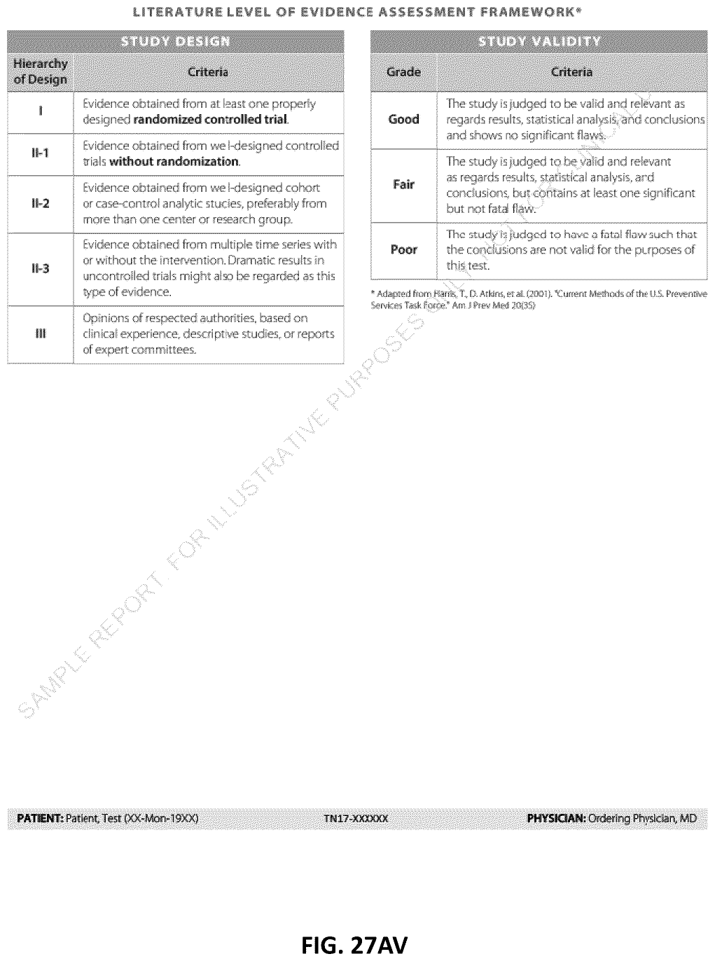

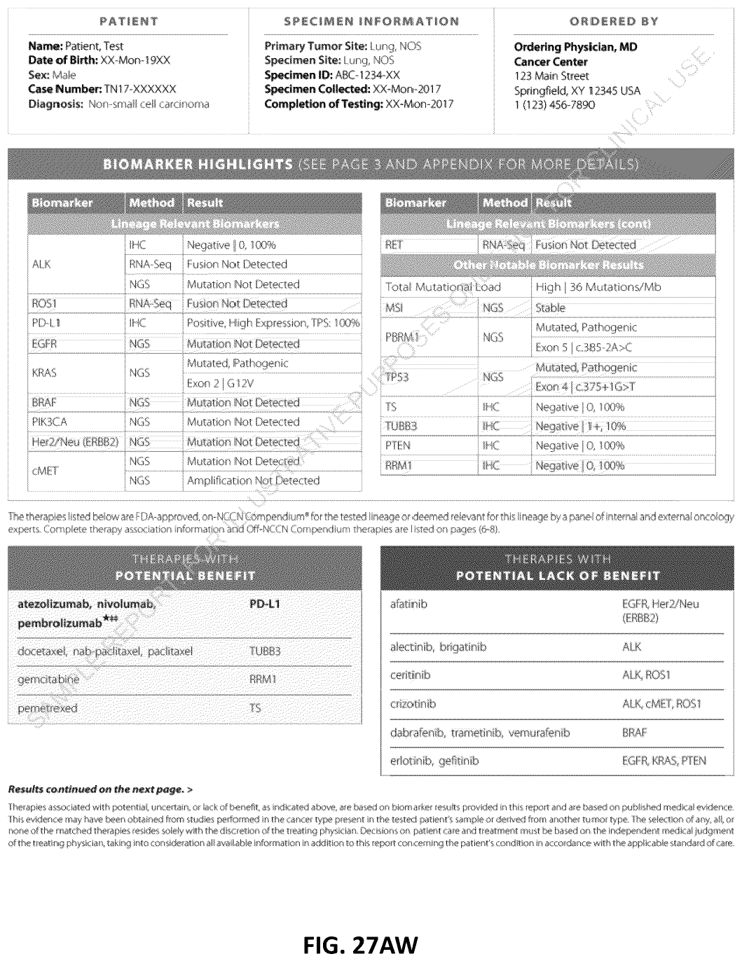

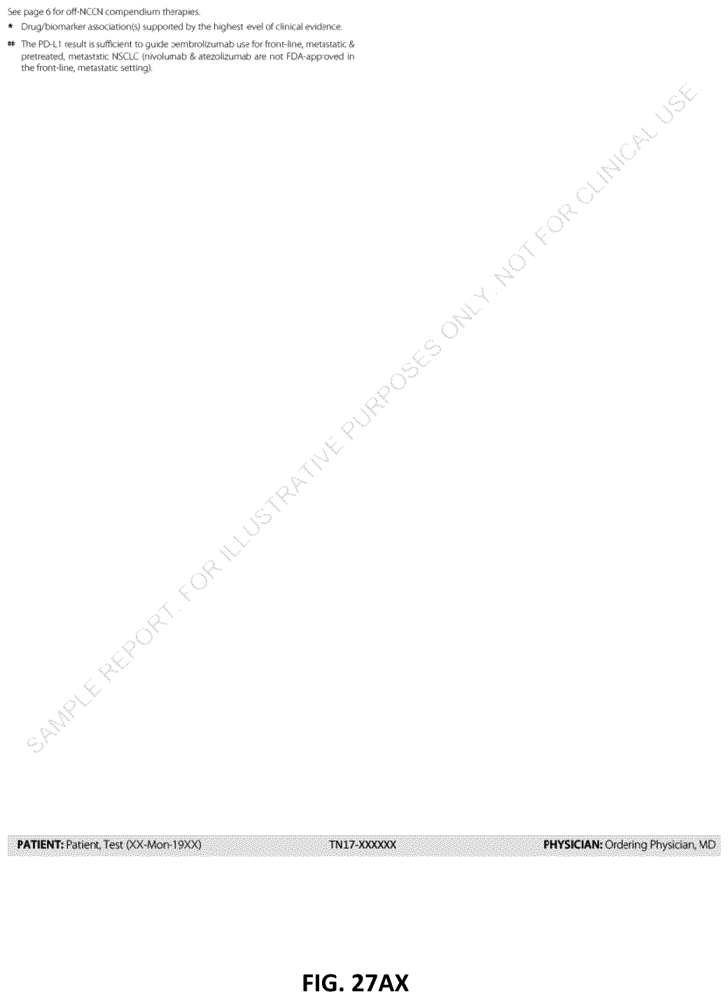

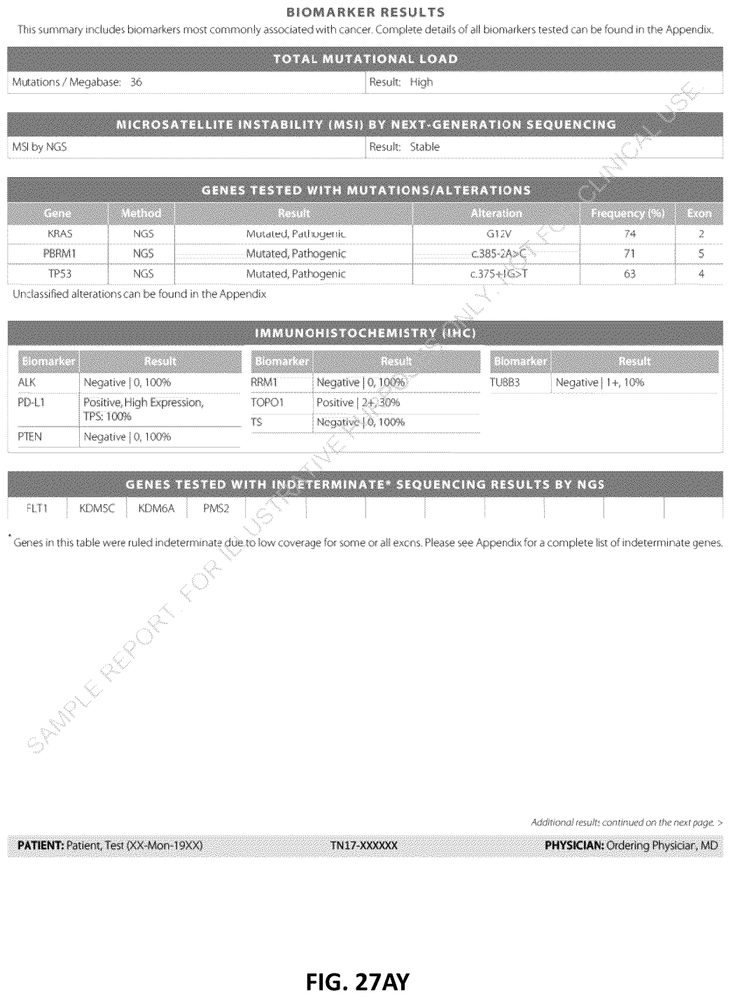

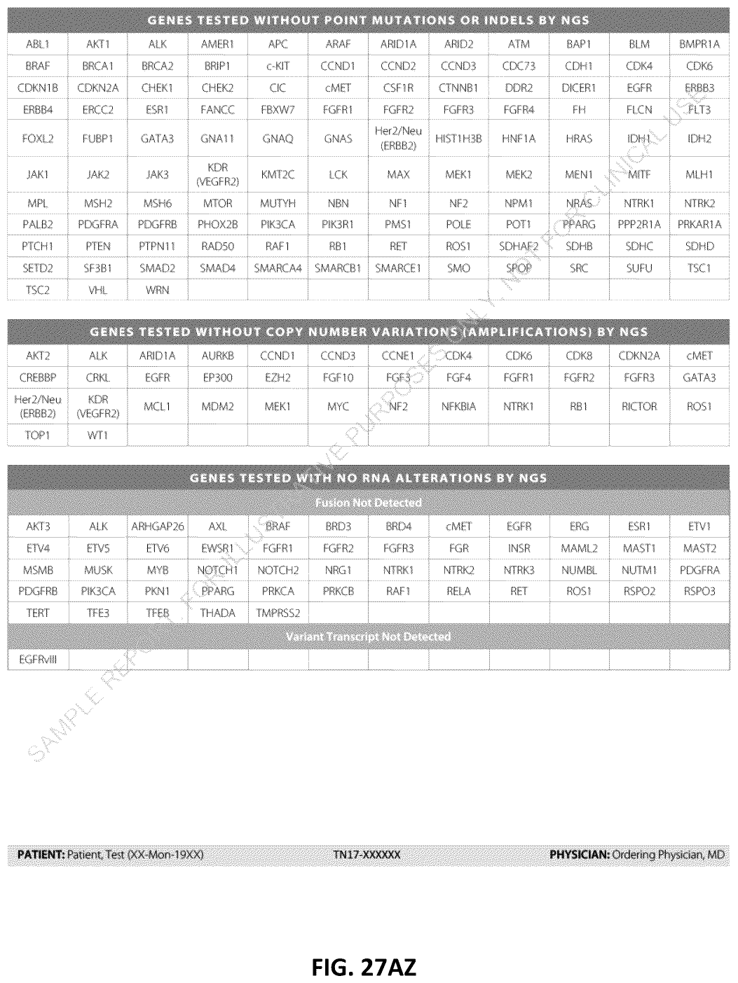

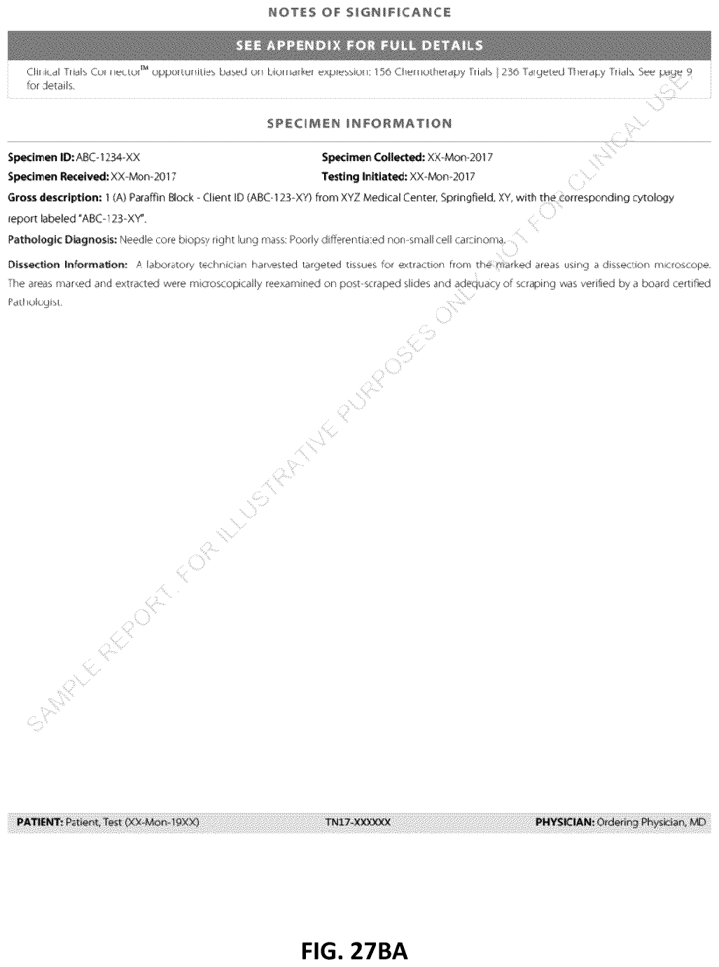

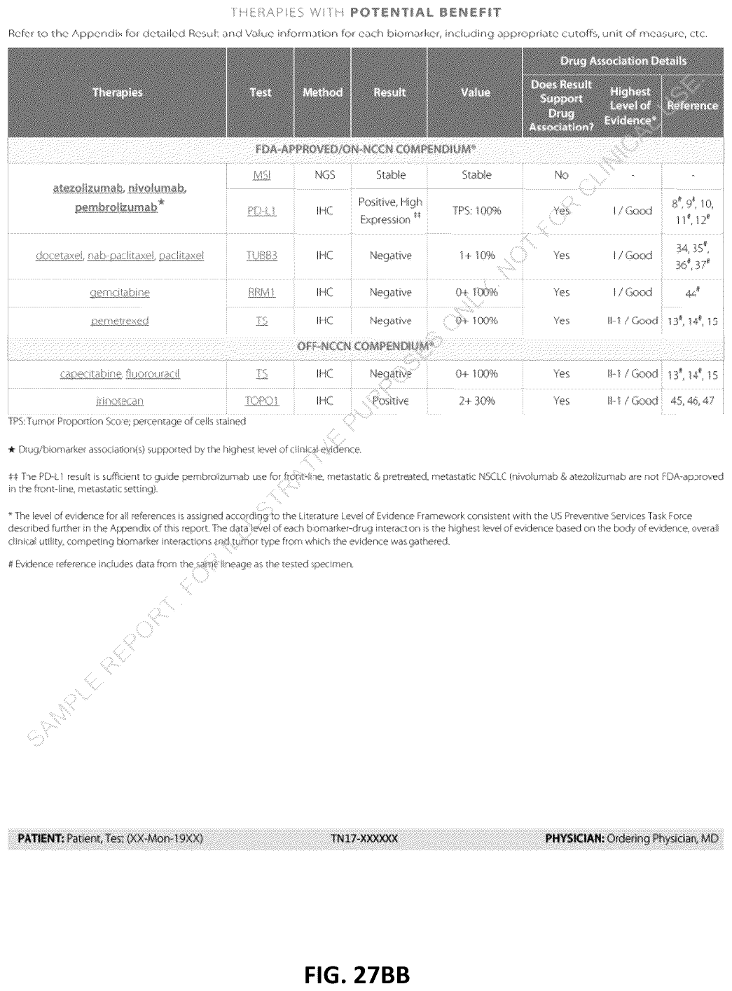

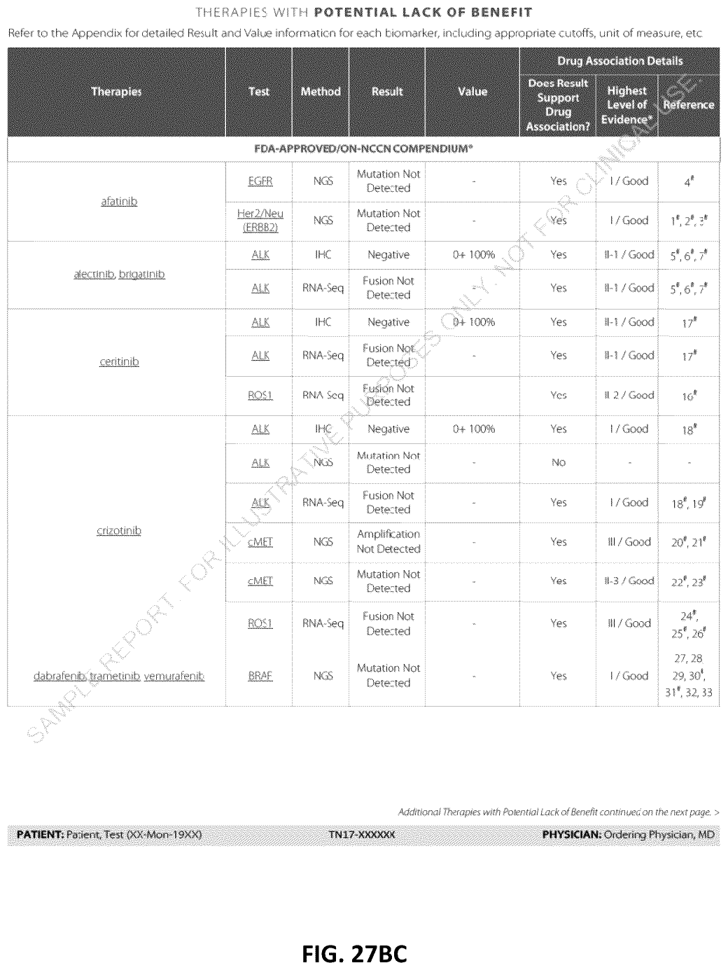

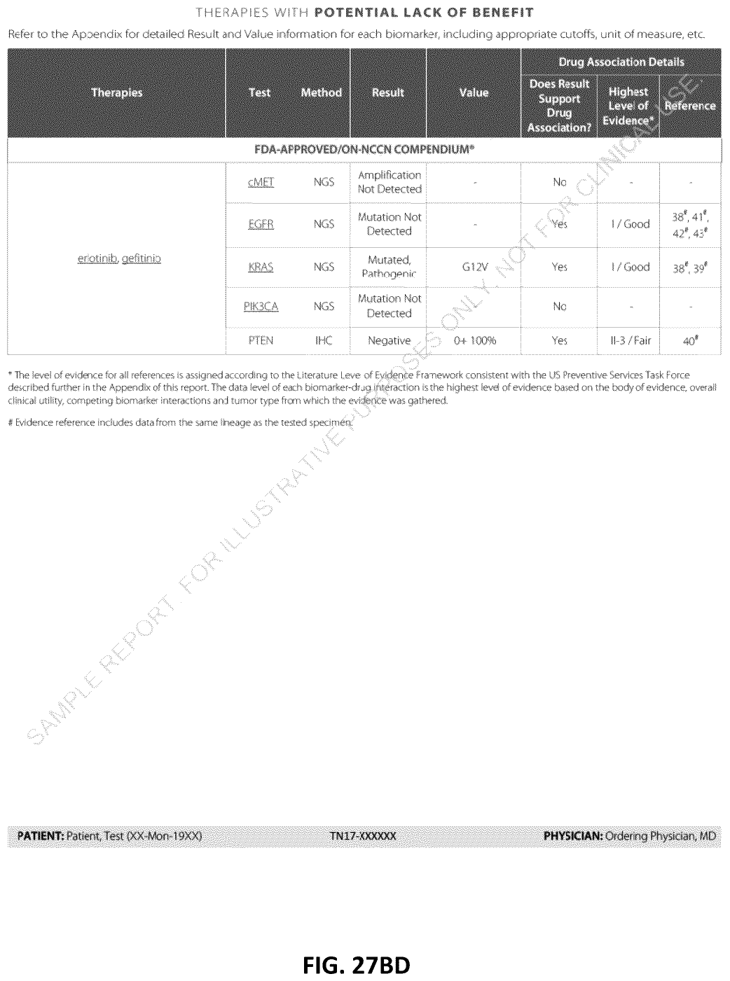

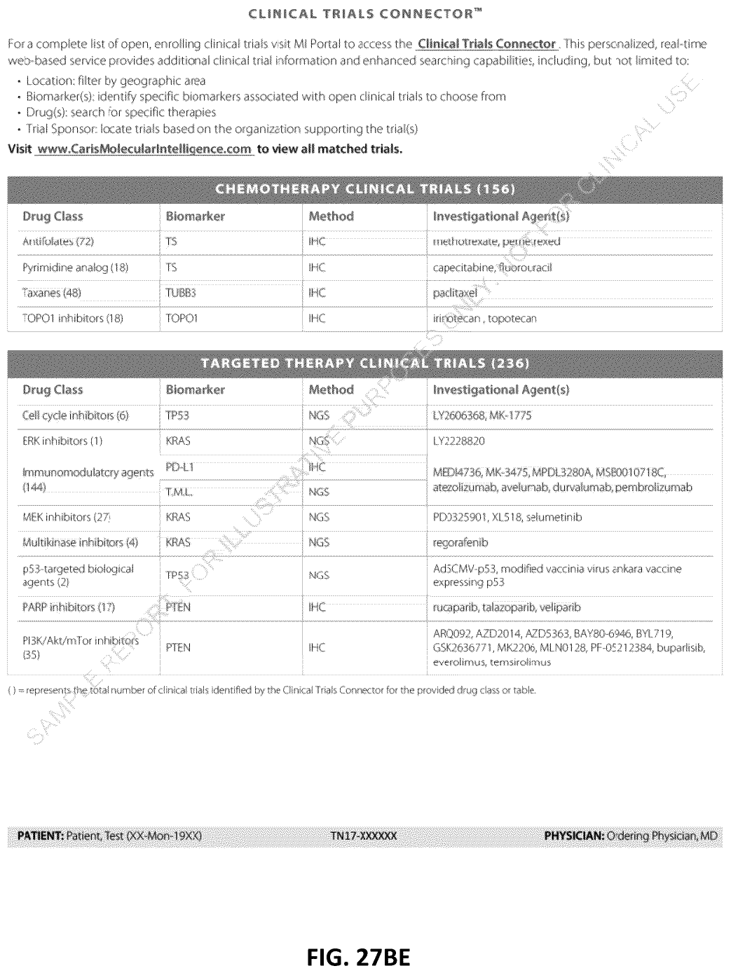

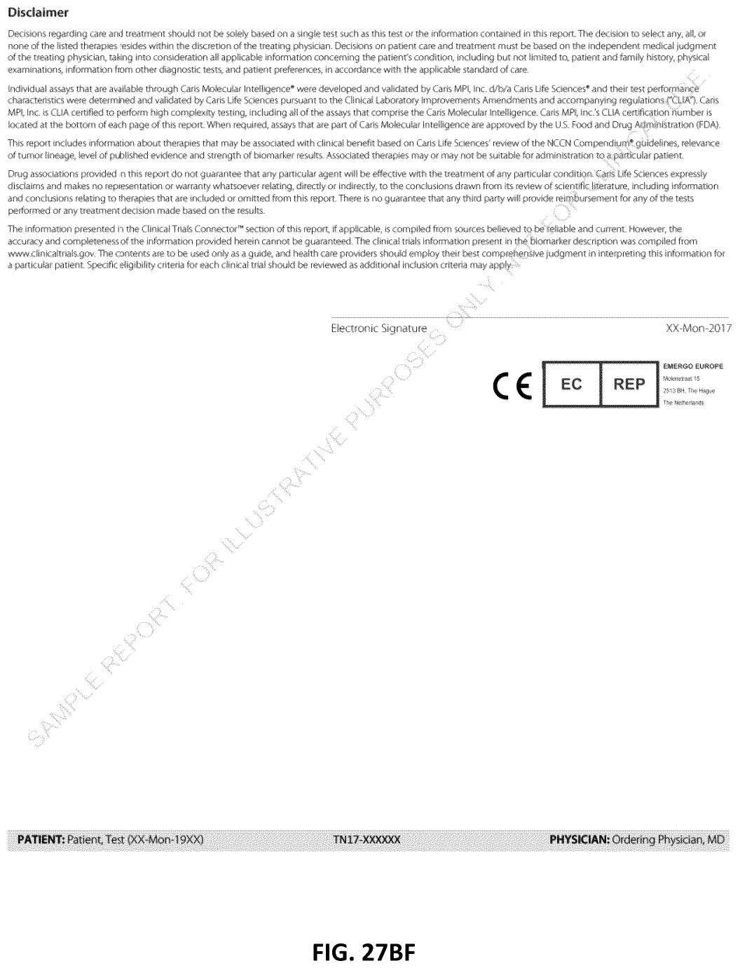

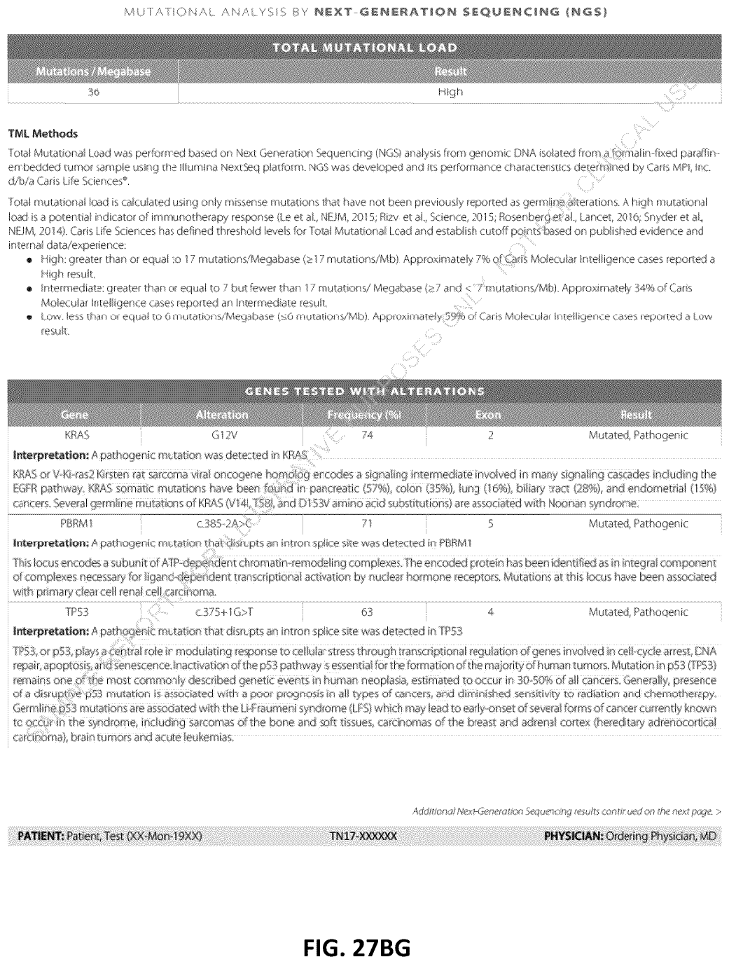

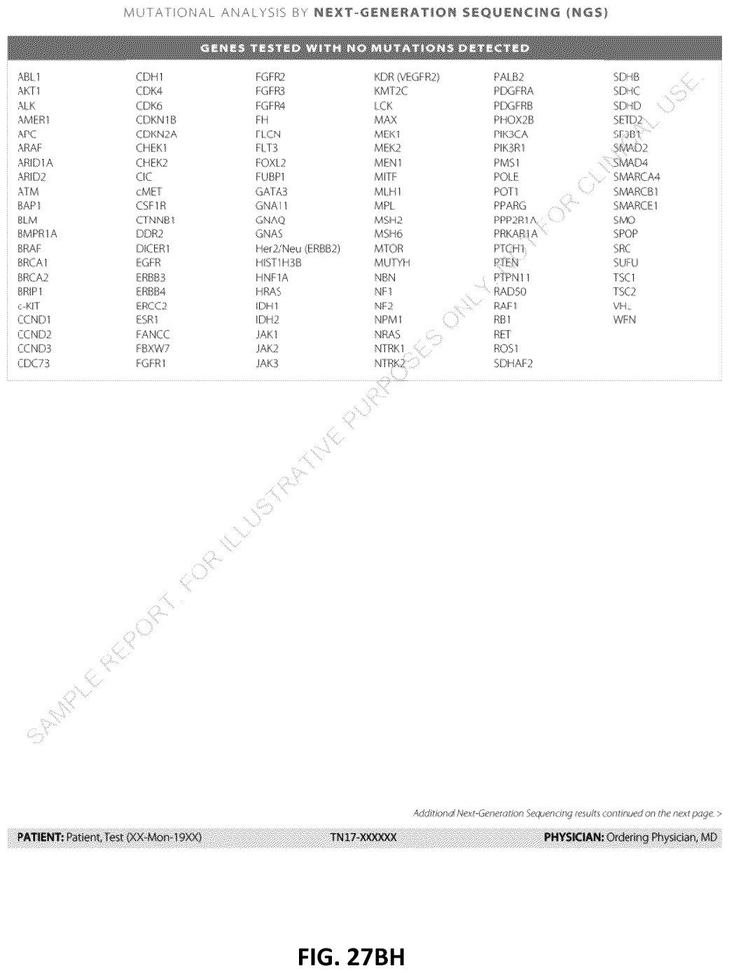

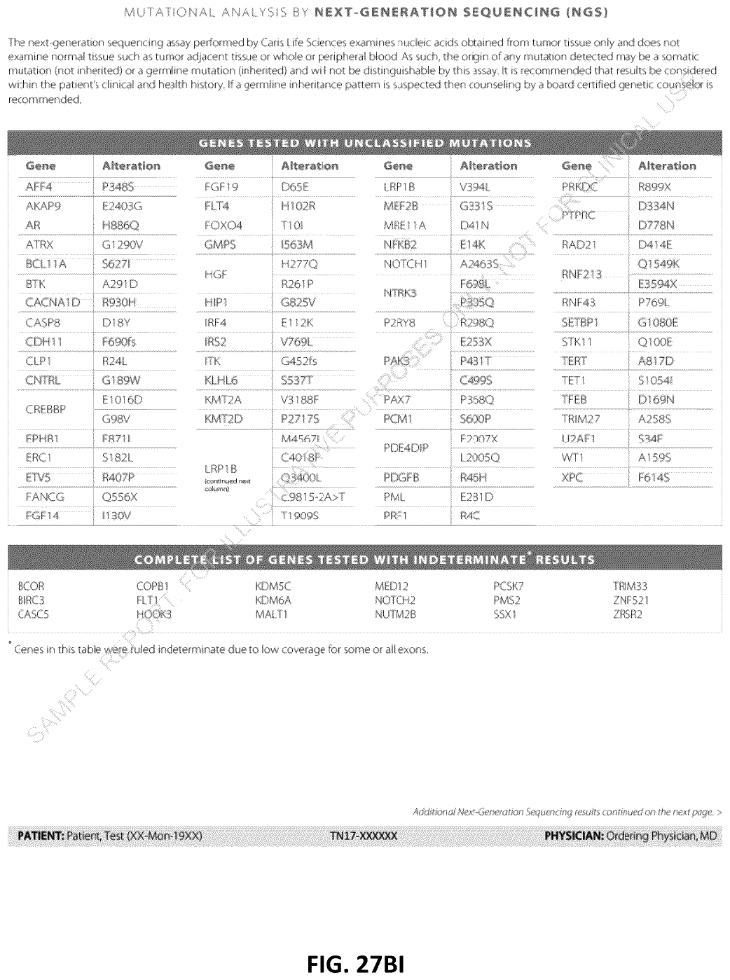

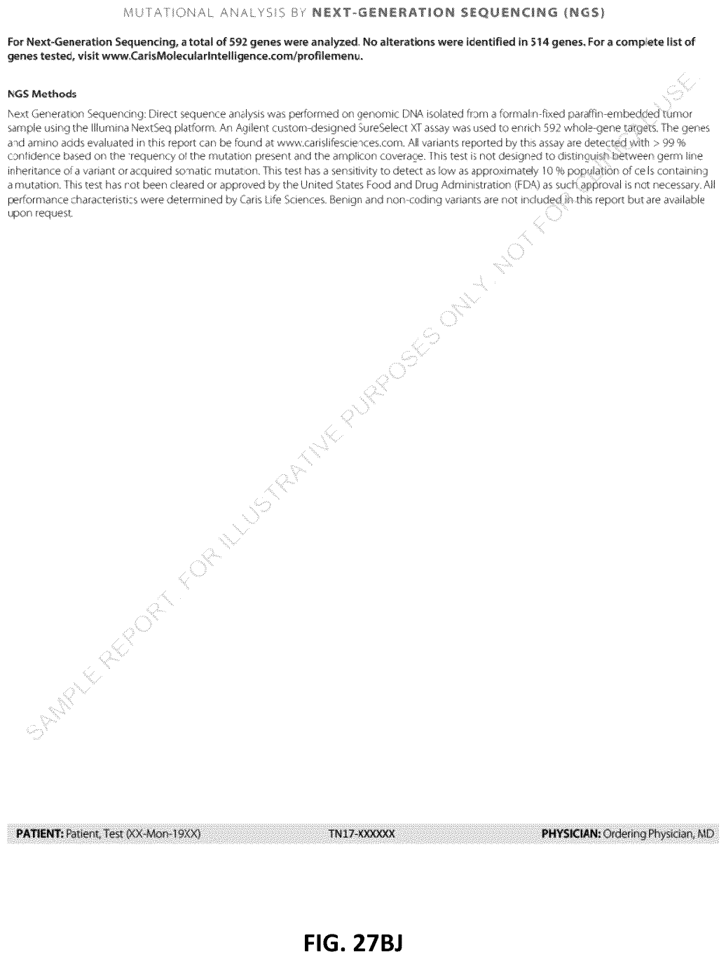

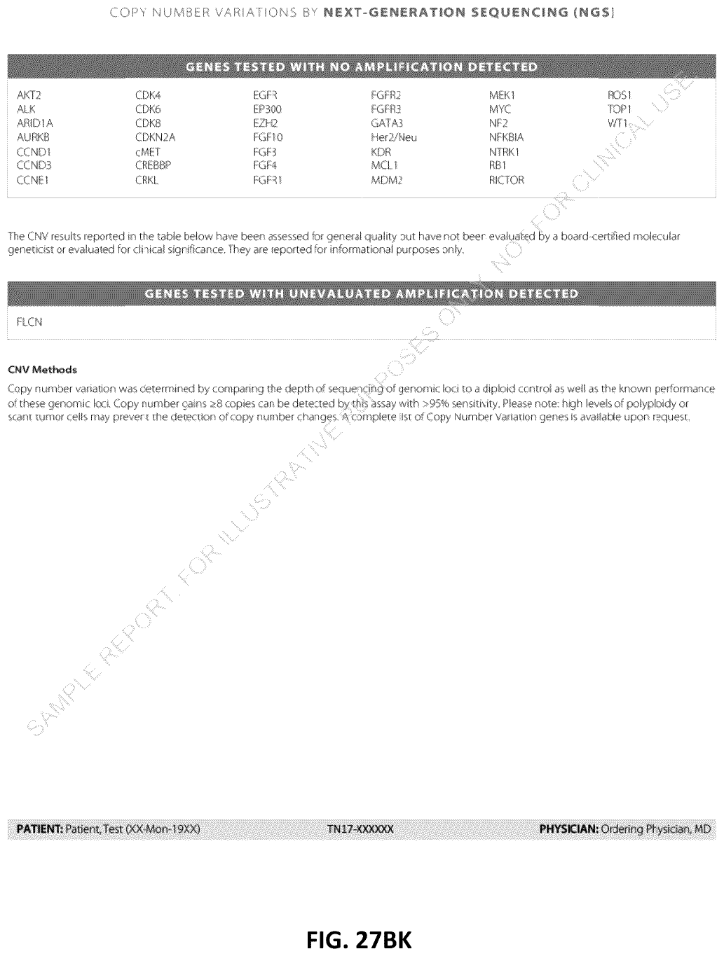

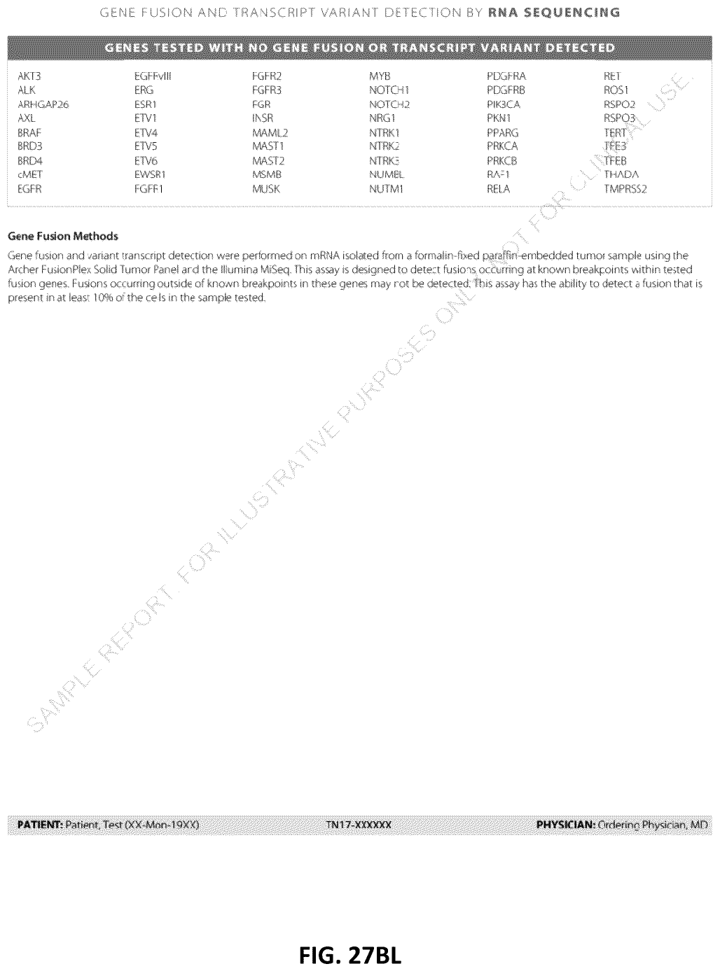

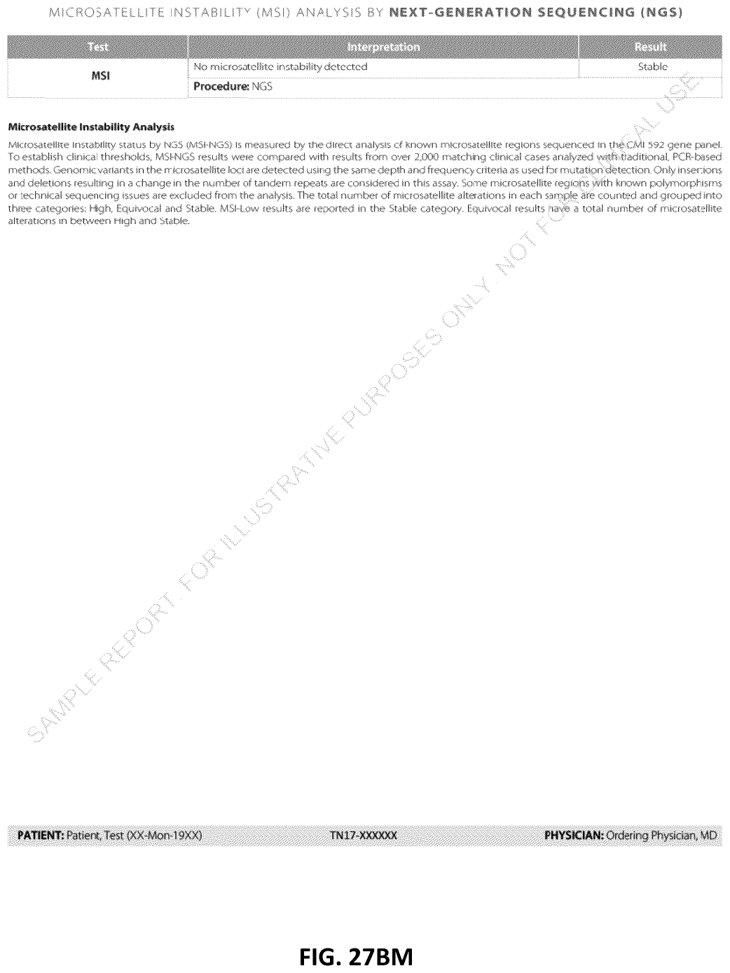

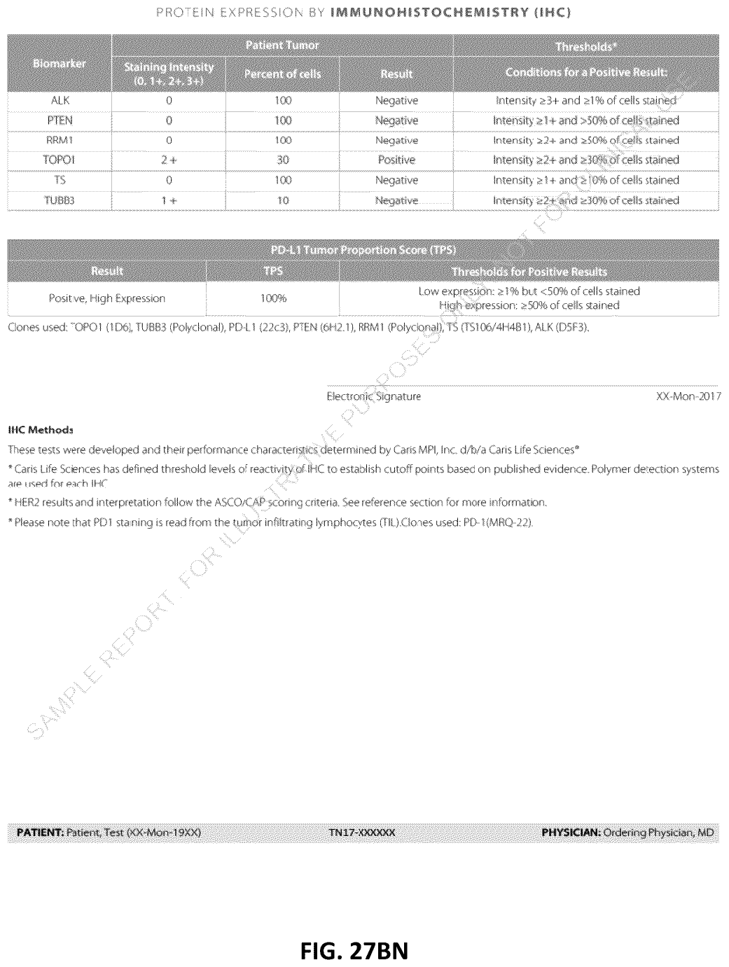

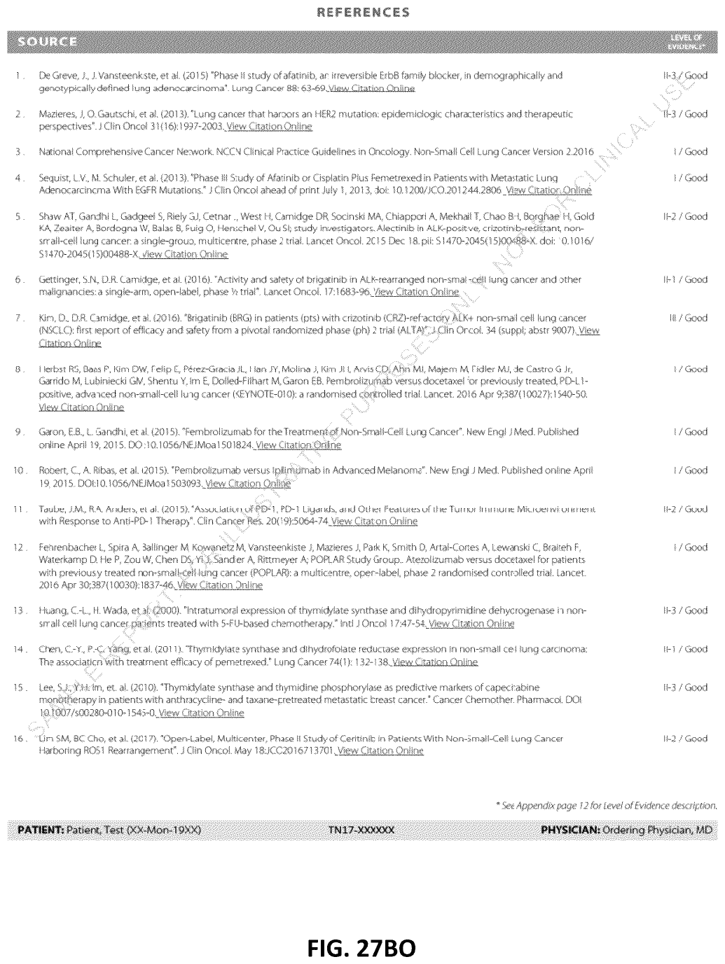

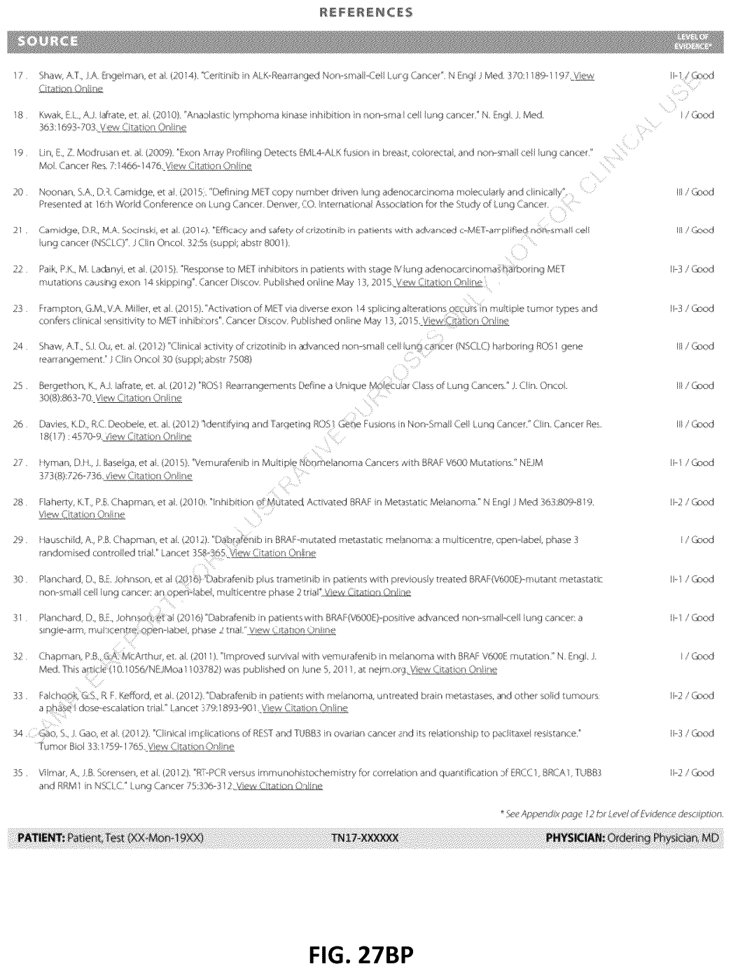

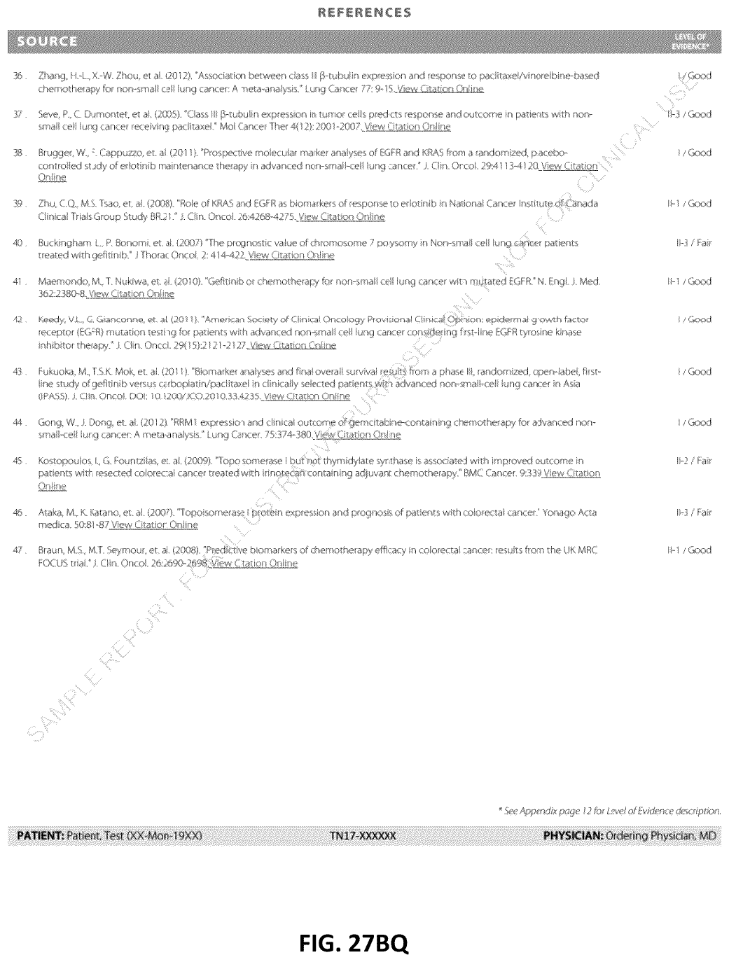

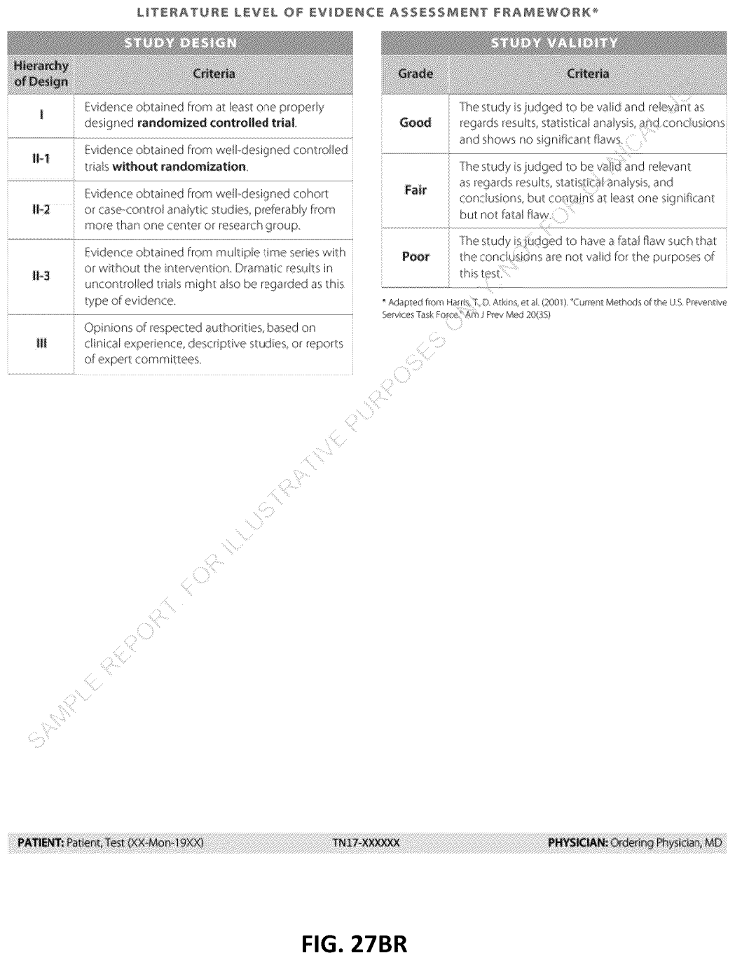

FIGS. 27A-BR herein present three illustrative patient reports according to the invention. FIGS. 27A-27Z provide an illustrative molecular profiling report derived from molecular profiling of a breast cancer. FIGS. 27AA-AV provide an illustrative molecular profiling report derived from molecular profiling of a colorectal cancer. FIGS. 27AW-BR provide an illustrative molecular profiling report derived from molecular profiling of a lung cancer (NSCLC). In all cases, the reports are for actual patients and are de-identified.

As noted herein, the same biomarker may be assessed by one or more technique. In such cases, the results of the different analysis may be prioritized in case of inconsistent results. For example, the different methods may detect different aspects of a single biomarker (e.g., expression level versus mutation), or one method may be more sensitive than another. In one example, consider that molecular profiling results obtained using the FDA approved cobas PCR (Roche Diagnostics) can be prioritized over Next Generation sequencing results. However, if the sequencing detects a mutation, e.g., V600E, V600E2 or V600K, when PCR either detects wild type or is not determinable, the report may contain a note describing both sets of results including any therapy that may be implicated. In the case of melanoma, when the result of BRAF cobas PCR is “Wild type” or “no data” whereas BRAF sequencing is “V600E” or “V600E2”, the report may comprise a note that BRAF mutation was not detected by the FDA-approved Cobas PCR test, however, a V600E/E2 mutation was detected by alternative methods (next generation/Sanger sequencing) and that evidence suggests that the presence of a V600E mutation associates with potential clinical benefit from vemurafenib, dabrafenib or trametinib therapy. Similarly, when the result of BRAF cobas PCR is “Wild type” or “no data” and BRAF sequencing is “V600K”, the report may comprise a note that BRAF mutation was not detected by the FDA-approved Cobas PCR test, however, a V600K mutation was detected by alternative methods (next generation/Sanger sequencing) and that evidence suggests that the presence of a V600K mutation associates with potential clinical benefit from trametinib therapy.

The molecular profiling report can be delivered to the caregiver for the subject, e.g., the oncologist or other treating physician. The caregiver can use the results of the report to guide a treatment regimen for the subject. For example, the caregiver may use one or more treatments indicated as likely benefit in the report to treat the patient. Similarly, the caregiver may avoid treating the patient with one or more treatments indicated as likely lack of benefit in the report.

### Immune Modulators

PD1 (programmed death-1, PD-1) is a transmembrane glycoprotein receptor that is expressed on CD4-/CD8-thymocytes in transition to CD4+/CD8+stage and on mature T and B cells upon activation. It is also present on activated myeloid lineage cells such as monocytes, dendritic cells and NK cells. In normal tissues, PD-1 signaling in T cells regulates immune responses to diminish damage, and counteracts the development of autoimmunity by promoting tolerance to self-antigens. PD-L1 (programmed cell death 1 ligand 1, PDL1, cluster of differentiation 274, CD274, B7 homolog 1, B7-H1, B7H1) and PD-L2 (programmed cell death 1 ligand 2, PDL2, B7-DC, B7DC, CD273, cluster of differentiation 273) are PD1 ligands. PD-L1 is constitutively expressed in many human cancers including without limitation melanoma, ovarian cancer, lung cancer, clear cell renal cell carcinoma (CRCC), urothelial carcinoma, HNSCC, and esophageal cancer. Blockade of PD-1 which is expressed in tumor-infiltrating T cells (TILs) has created an important rationale for development to monoclonal antibody therapy to target blockade of PD1/PDL-1 pathway. Tumor cell expression of PD-L1 is used as a mechanism to evade recognition/destruction by the immune system as in normal cells the PD1/PDL1 interplay is an immune checkpoint. Monoclonal antibodies targeting PD-1/PD-L1 that boost the immune system are being developed for the treatment of cancer. See, e.g., Flies et al, Blockade of the B7-H1/PD-1 pathway for cancer immunotherapy. Yale J Biol Med. 2011 December; 84(4):409-21; Sznol and Chen, Antagonist Antibodies to PD-1 and B7-H1 (PD-L1) in the Treatment of Advanced Human Cancer, Clin Cancer Res; 19(5) Mar. 1, 2013; Momtaz and Postow, Immunologic checkpoints in cancer therapy: focus on the programmed death-1 (PD-1) receptor pathway. Pharmgenomics Pers Med. 2014 Nov. 15; 7:357-65; Shin and Ribas, The evolution of checkpoint blockade as a cancer therapy: what's here, what's next?, Curr Opin Immunol. 2015 Jan. 23; 33C:23-35; which references are incorporated by reference herein in their entirety. Several drugs are in clinical development that affect the PDL1/PD1 pathway include: 1) Nivolumab (BMS936558/MDX-1106), an anti-PD1 drug from Bristol Myers Squib drug which was approved by the U.S. FDA in late 2014 under the brand name OPDIVO for the treatment of patients with unresectable or metastatic melanoma and disease progression following ipilimumab and, if BRAF V600 mutation positive, a BRAF inhibitor; 2) Pembrolizumab (formerly lambrolizumab, MK-3475, trade name Keytruda), an anti-PD1 drug from Merck approved in late 2014 for use following treatment with ipilimumab, or after treatment with ipilimumab and a BRAF inhibitor in patients who carry a BRAF mutation; 3) BMS-936559/MDX-1105, an anti-PDL1 drug from Bristol Myers Squib with initial evidence in advanced solid tumors; and 4) MPDL3280A, an anti-PDL1 drug from Roche with initial evidence in NSCLC.

Expression of PD1, PD-L1 and/or PD-L2 expression can be assessed at the protein and/or mRNA level according to the methods of the invention. For example, IHC can be used to assess their protein expression. Expression may indicate likely benefit of inhibitors of the B7-H1/PD-1 pathway, whereas lack of expression may indicate lack of benefit thereof. In some embodiments, expression of both PD-1 and PD-L1 is assessed and likely benefit of inhibitors of the B7-H1/PD-1 pathway is determined only upon co-expression of both of these immunosuppressive components. Certain cells express PD-L1 mRNA, but not the protein, due to translational suppression by microRNA miR-513. Therefore, analysis of PD-L1 protein may be desirable for molecular profiling. Molecular profiling may also include that of miR-513. Expression of miR-513 above a certain threshold may indicate lack of benefit of immune modulation therapy.

In an aspect, the invention provides a method of identifying at least one treatment associated with a cancer in a subject, comprising: a) determining a molecular profile for at least one sample from the subject by assessing a plurality of gene or gene products, wherein the plurality of genes and/or gene products comprises at least one of PD-1 and PD-L1; and b) identifying, based on the molecular profile, at least one of: i) at least one treatment that is associated with benefit for treatment of the cancer; ii) at least one treatment that is associated with lack of benefit for treatment of the cancer; and iii) at least one treatment associated with a clinical trial. Expression of PD-1 and/or PD-L1 may be performed along with that of additional biomarkers that guide treatment selection according to the invention. Such additional biomarkers can be additional immune modulators including without limitation CTL4A, IDO1, COX2, CD80, CD86, CD8A, Granzyme A, Granzyme B, CD19, CCR7, CD276, LAG-3, TIM-3, and a combination thereof. The additional biomarkers could also comprise other useful biomarkers disclosed herein, such any of Tables 2-12. For example, the additional biomarkers may comprise at least one of 1p19q, ABL1, AKT1, ALK, APC, AR, ATM, BRAF, BRCA1, BRCA2, cKIT, cMET, CSF1R, CTNNB1, EGFR, EGFRvIII, ER, ERBB2 (HER2), FGFR1, FGFR2, FLT3, GNA11, GNAQ, GNAS, HER2, HRAS, IDH1, IDH2, JAK2, KDR (VEGFR2), KRAS, MGMT, MGMT-Me, MLH1, MPL, NOTCHI, NRAS, PDGFRA, Pgp, PIK3CA, PR, PTEN, RET, RRM1, SMO, SPARC, TLE3, TOP2A, TOPO1, TP53, TS, TUBB3, VHL, CDH1, ERBB4, FBXW7, HNF1A, JAK3, NPM1, PTPN11, RBi, SMAD4, SMARCB1, STK1, MLH1, MSH2, MSH6, PMS2, microsatellite instability (MSI), ROS1 and ERCC1. These additional analyses may suggest combinations of therapies likely to benefit the patient, such as a PD-1/PD-L1 pathway inhibitor and another therapy suggested by the molecular profiling. See, e.g., additional biomarker-drug associations in any of Tables 2-3, Table 11. In some embodiments, anti-CTLA-4 therapy, including without limitation ipilimumab, is administered with PD-1/PD-L1 pathway therapy.

The invention further provides association of immune modulation therapy, including without limitation PD-1/PD-L1 pathway inhibitor treatments, with molecular profiling of biomarkers in addition to PD-1/PD-L1 themselves. In an embodiment of the invention, beneficial treatment of the cancer with immunotherapy targeting at least one of PD-1, PD-L1, CTLA-4, IDO-1, and CD276, is associated with a molecular profile indicating that the cancer is AR-/HER2-/ER-/PR-(quadruple negative) and/or carries a mutation in BRCA1. In some embodiments, the invention provides associating beneficial treatment of the cancer with immunotherapy targeting immune modulating therapy wherein the molecular profile indicates that the cancer carries a mutation in at least one cancer-related gene. The cancer-related gene can include at least one, e.g., at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46 or 47, of ABL1, AKT1, ALK, APC, ATM, BRAF, BRCA1, BRCA2, cKIT, cMET, CSF1R, CTNNB1, EGFR, ERBB2, FGFR1, FGFR2, FLT3, GNA11, GNAQ, GNAS, HRAS, IDH1, JAK2, KDR (VEGFR2), KRAS, MLH1, MPL, NOTCHI, NRAS, PDGFRA, PIK3CA, PTEN, RET, SMO, TP53, VHL, CDH1, ERBB4, FBXW7, HNF1A, JAK3, NPM1, PTPN11, RBi, SMAD4, SMARCB1 and STK1. Other cancer related genes, such as those disclosed herein or in the COSMIC (Catalogue Of Somatic Mutations In Cancer) database (available at cancer.sanger.ac.uk/cancergenome/projects/cosmic/), can be assessed as well. See Tables 7-10 for additional genes that can be assessed. It will be apparent to one of skill that such profiling may be performed independently of direct assessment of immune modulators themselves. As an illustrative example, a tumor determined to carry a mutation in BRCA1 may be a candidate for anti-PD-1 and/or anti-PD-L1 therapy. Thus, in a related aspect, the invention provides a method of identifying at least one treatment associated with a cancer in a subject, comprising: a) determining a molecular profile for at least one sample from the subject by assessing a plurality of genes and/or gene products other than PD-1 and/or PD-L1; and b) identifying, based on the molecular profile, that the cancer is likely to benefit from anti-PD-1 or anti-PD-L1 therapy.

Expression of PD-1 is generally assessed in tumor infiltrating lymphocytes (TILs). PD-L1 may be expressed in various cells in the tumor microenvironment. In addition to tumor cells, PD-L1 can be expressed by T cells, natural killer (NK) cells, macrophages, myeloid dendritic cells (DCs), B cells, epithelial cells, and vascular endothelial cells. In some cases, the response to anti-PD-1/PD-L1 therapy may be dependent on which cells in the tumor microenvironment express PD-L1. Thus, in some embodiments of the invention, the tumor microenvironment is assessed to determine the expression patterns of PD-L1 and the likely benefit or lack thereof is dependent on the cells determined to express PD-L1. Such PD-L1 expression can be determined in various cells, including without limitation one or more of T cells, natural killer (NK) cells, macrophages, myeloid dendritic cells (DCs), B cells, epithelial cells, and endothelial cells.

Certain tumor cells may also more susceptible to immune modulating therapy and thus more likely associated with likely treatment benefit. An “immune modulating therapy” can include antagonists such as antibodies to PD-1, PD-L1, PD-L2, CTL4A, IDO1, COX2, CD80, CD86, CD8A, Granzyme A, Granzyme B, CD19, CCR7, CD276, LAG-3 or TIM-3. The antagonist could also be a soluble ligand or small molecule inhibitor. As a non-limiting example, a soluble PD-L1 construct may bind PD-1 and thus block its immunosuppressive activity. In an embodiment, the invention provides for determining the apoptotic or necrotic environment of the tumor. Apoptotic or necrotic cells may be associated with likely treatment benefit from immune modulating therapy. Thus, the invention provides a method of identifying at least one treatment associated with a cancer in a subject, comprising: a) determining a molecular profile for at least one sample from the subject by assessing tumor necrosis or apoptosis; and b) associating the cancer with likely to benefit from immune modulating therapy, including without limitation anti-PD-1 or anti-PD-L1 therapy, if apoptotic or necrotic tumor cells are identified.

### Genomic Stability Profiling

Microsatellites are repeated sequences of DNA. These sequences can be made of repeating units of one to six base pairs in length. Although the length of these microsatellites is highly variable from person to person and contributes to the individual DNA fingerprint, each individual has microsatellites of a set length.

Microsatellite instability (MSI) is the condition of genetic hypermutability that results from impaired DNA mismatch repair (MMR). Deficient MMR may be referred to as dMMR. MSI may be caused by hypermutation of the MLH1 gene, or by mutations in MMR genes such as MLH1, MSH2, MSH6, and PMS2. The presence of MSI represents phenotypic evidence that MMR is not functioning normally. Microsatellite instability may be found in any variety of cancer, including without limitation colon cancer, gastric cancer, endometrium cancer, ovarian cancer, hepatobiliary tract cancer, urinary tract cancer, brain cancer, and skin cancers. MSI is most prevalent as the cause of colon cancers.

The NCI has agreed on five microsatellite markers as the godl standard to determine MSI presence: two mononucelotides, BAT25 and BAT26, and three dinucelotide repeats, D2S123, D5S346, and D17S250. MSI-High (MSI-H) tumors result from MSI of greater than 30% of unstable MSI biomarkers. MSI-Low (MSI-L) tumors result from less than 30% of unstable MSI biomarkers. MSI-L tumors are classified as tumors of alternative etiologies. Several studies demonstrate that MSI-H patients respond best to surgery alone, rather than chemotherapy and surgery, thus preventing patients from needlessly experiencing chemotherapy. Recently it has been found that MSI status can affect response to immune therapy. For example, PD-1 blockade was more effective against MSI-high tumors than against microsatellite-stable tumors. See Le et al. PD-1 blockade in tumors with mismatch-repair deficiency. N Engl J Med 2015 Jun 25; 372:2509; Int'l Patent Publication WO2016077553A1 to Diaz et al entitled “Checkpoint blockade and microsatellite instability”; which references are incorporated by reference herein in their entirety.

High tumor mutational load (TML; or tumor mutation burden, TMB) is another recently identified biomarker that is a potential indicator of immunotherapy response. See, e.g., Le et al., PD-1 Blockade in Tumors with Mismatch-Repair Deficiency, N Engl J Med 2015; 372:2509-2520; Rizvi et al., Mutational landscape determines sensitivity to PD-1 blockade in non-small cell lung cancer. Science. 2015 Apr. 3; 348(6230): 124-128; Rosenberg et al., Atezolizumab in patients with locally advanced and metastatic urothelial carcinoma who have progressed following treatment with platinum-based chemotherapy: a single arm, phase 2 trial. Lancet. 2016 May 7; 387(10031): 1909-1920; Snyder et al., Genetic Basis for Clinical Response to CTLA-4 Blockade in Melanoma. N Engl J Med. 2014 Dec. 4; 371(23): 2189-2199; Int'l Patent Publication WO2016081947A2 to Chan et al entitled “Determinants of Cancer Response to Immunotherapy by PD-1 Blockade”; Int'l Patent Publication WO2017151524A1 to Frampton et al. entitled “Methods and Systems for Evaluating Tumor Mutational Burden”; all of which references are incorporated by reference herein in their entirety.

Immune checkpoints are regulators of the immune system. These pathways are crucial for self-tolerance, which prevents the immune system from attacking cells indiscriminately. Programmed death-1 (PD-1, CD279) is an immune suppressive molecule that is upregulated on activated T cells and other immune cells. It is activated by binding to its ligand PD-L1 (B7-H1, CD274), which results in intracellular responses that reduce T-cell activation. The PD1/PDL1 interplay is an immune checkpoint. Tumor cell expression of PD-L1 is used as a mechanism to evade recognition/destruction by the immune system. Aberrant PD-L1 expression had been observed on cancer cells, leading to the development of PD-1/PD-L1-directed cancer therapies. Checkpoint therapy includes agents that block PD-1/PD-L1 immune suppression. Blockade of the PD-1 and PD-L1 interaction has led to clinical responses in several cancer types. Clinically available examples of PD-L1 inhibitors include durvalumab, atezolizumab and avelumab. Cancer immunotherapy agents that target the PD-1 receptor include nivolumab, pembrolizumab, pidilizumab and BMS-936559.

The invention provides advantages over previous methods in determining biomarkers of genomic stability and immune checkpoint response. The systems and methods provided herein can be used to assess multiple biomarkers which provide complementary indications that checkpoint therapy may be of potential benefit to a cancer victim. See, e.g., Examples 7 and 8 herein. The systems and methods can be integrated into comprehensive molecular profiling to identify multiple potential therapies of benefit or potential lack of benefit for the cancer victim. See, e.g., Examples 1-6 herein.

In an aspect, the invention provides a method of determining microsatellite instability (MSI) in a biological sample, comprising: (a) obtaining a nucleic acid sequence of a plurality of microsatellite loci from the biological sample; (b) determining the number of altered microsatellite loci based on the nucleic acid sequences obtained in step (a); (c) comparing the number of altered microsatellite loci determined in step (b) to a threshold number; and (d) identifying the biological sample as MSI-high if the number of altered microsatellite loci is greater than or equal to the threshold number.

The biological sample can be any useful biological sample. In embodiments of the method of determining MSI, the biological sample comprises formalin-fixed paraffin-embedded (FFPE) tissue, fixed tissue, a core needle biopsy, a fine needle aspirate, unstained slides, fresh frozen (FF) tissue, formalin samples, tissue comprised in a solution that preserves nucleic acid or protein molecules, a fresh sample, a malignant fluid, a bodily fluid, a tumor sample, a tissue sample, or any combination thereof. In preferred embodiments, the biological sample comprises cells from a tumor, e.g., a solid tumor. The biological sample may comprise a bodily fluid. In some embodiments, the bodily fluid comprises a malignant fluid, a pleural fluid, a peritoneal fluid, or any combination thereof. In some embodiments, the bodily fluid comprises peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid, pre-ejaculatory fluid, female ejaculate, sweat, fecal matter, tears, cyst fluid, pleural fluid, peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyst cavity fluid, or umbilical cord blood. The sample may comprise microvesicles.

In embodiments of the method of determining MSI, the nucleic acid sequence is obtained by sequencing DNA or RNA. In preferred embodiments, the DNA is genomic DNA. For example, genomic DNA from the biological sample can be sequenced. The sequencing can be any useful sequencing method, preferably high throughput sequencing, also referred to as next generation sequencing (NGS), in order to efficiently assess multiple loci.

In embodiments of the method of determining MSI, the plurality of microsatellite loci comprises any useful number of loci, including without limitation at least 5, 10, 15, 20, 25, 30, 35, 40, 45, 50, 55, 60, 65, 70, 75, 80, 85, 90, 95, 100, 125, 150, 175, 200, 250, 300, 350, 400, 450, 500, 550, 600, 650, 700, 750, 800, 850, 900, 950, 1000, 2000, 3000, 4000, 5000, 6000, 7000, 8000, 9000 or 10000 loci. The plurality of microsatellite loci can be filtered to exclude loci meeting certain criteria. In preferred embodiments, the plurality of microsatellite loci excludes: i) sex chromosome loci; ii) microsatellite loci in regions that typically have lower coverage depth relative to other genomic regions; iii) microsatellites with repeat unit lengths greater than 3, 4, 5, 6 or 7 nucleotides, preferably greater than 5 nucleotides; or iv) any combination of i)-iii). In regards to ii), the coverage depth (also known as sequencing depth or read depth) describes the number of times that a given nucleotide in the genome has been read in an experiment. Greater number of reads can lead to better sequencing results. Thus, the method may favor analysis of higher quality sequences with greater sequencing depth.

In some embodiments, the members of the plurality of microsatellite loci are selected from Table 16. For example, the plurality of microsatellite loci may comprise all loci in Table 16, or the plurality of loci may consist of all loci in Table 16. In other embodiments, the plurality of microsatellite loci comprise certain loci from Table 16 and other additional loci that meet desired criteria. The members of the plurality of microsatellite loci can be chosen based on certain desired criteria. In some embodiments, the members of the plurality of microsatellite loci are located within the vicinity of a gene. In preferred embodiments, each member of the plurality of microsatellite loci is located within the vicinity of a cancer gene. For example, each member of the plurality of microsatellite loci can be located within the vicinity of a cancer gene selected from Table 7, Table 8, Table 9, Table 10, or any combination thereof. Accordingly, mutations, indels, CNV, fusions, and the like can be detected in a panel of cancer genes, and the same sequencing runs can be used to assess MSI.

In embodiments of the method of determining MSI, determining the number of altered microsatellite loci in step (b) comprises comparing each nucleic acid sequence obtained in step (a) to a reference sequence for each microsatellite loci. For example, the reference sequence can be a human genomic reference sequence, including without limitation those provided by the UCSC Genome Browser or Ensembl genome browser projects. Determining the number of altered microsatellite loci may comprise identifying microsatellites with insertions or deletions that increased or decreased the number of repeats in the microsatellite as compared to the reference sequence. In some embodiments, the number of altered microsatellite loci only counts each altered loci once regardless of the number of insertions or deletions at that loci. For example, a microsatellite with two inserted repeats as compared to the reference sequence would only be counted once in determining the number of altered microsatellite loci.

In embodiments of the method of determining MSI, the threshold number is calibrated based on comparison of the number of altered microsatellite loci per patient to MSI results obtained using a different laboratory technique on a same biological sample. The “same biological sample” can refer to any appropriate sample, such as the same physical sample, another portion of the same tumor, or less preferred a related tumor from the same individual. In some embodiments, the different laboratory technique comprises fragment analysis, immunohistochemistry of mismatch repair genes, immunohistochemistry of immunomodulators, or any combination thereof. In preferred embodiments, the different laboratory technique comprises the gold standard fragment analysis as described herein. The threshold number can be determined using any number of desired biological samples, including biological samples from at least 5, 10, 15, 20, 25, 30, 35, 40, 45, 50, 55, 60, 65, 70, 75, 80, 85, 90, 95, 100, 125, 150, 175, 200, 250, 300, 350, 400, 450, 500, 550, 600, 650, 700, 750, 800, 850, 900, 950, 1000, or 2000 different cancer patients. The samples can represent various cancers, e.g., at least 1, 2, 3, 4, 5, 6, 7, 8, 9, 10, 11, 12, 13, 14, 15, 20, 25, or 30 distinct cancer lineages. In some embodiments, the distinct cancer lineages comprise cancers selected from colorectal adenocarcinoma, endometrial cancer, bladder cancer, breast carcinoma, cervical cancer, cholangiocarcinoma, esophageal and esophagogastric junction carcinoma, extrahepatic bile duct adenocarcinoma, gastric adenocarcinoma, gastrointestinal stromal tumors, glioblastoma, liver hepatocellular carcinoma, lymphoma, malignant solitary fibrous tumor of the pleura, melanoma, neuroendocrine tumors, NSCLC, female genital tract malignancy, ovarian surface epithelial carcinomas, pancreatic adenocarcinoma, prostatic adenocarcinoma, small intestinal malignancies, soft tissue tumors, thyroid carcinoma, uterine sarcoma, uveal melanoma, and any combination thereof. In some embodiments, the threshold number is calibrated across at least 1, 2, 3, 4, 5, 6, 7, 8, 9, 10, 11, 12, 13, 14, 15, 20, or 25 distinct cancer lineages using sensitivity, specificity, positive predictive value, negative predictive value, or any combination thereof. For example, the threshold can be tuned with high sensitivity to MSI-high to reduce false negatives, or high specificity to MSI-high to reduce false positives, or any desired balance between.

In a preferred embodiment, the threshold number is set to provide high sensitivity to MSI-high as determined in colorectal cancer using the different laboratory technique, which different laboratory technique can be fragment analysis.

The threshold number will be related to the number and characteristics of the interrogated microsatellite loci. The threshold can be recalibrated, e.g., if a different set of loci are chosen. If relevant data is available, the threshold can be calibrated for different settings, such as different clinical criteria. For example, a different threshold may be calculated for different cancer lineages. In other embodiments, the threshold may be calibrated for different patient characteristics such as sex, age, clinical history including prior disease and treatments. Calibrating the threshold for different settings may rely on having sufficient data available to tune sensitivity, specificity, positive predictive value, negative predictive value, or other criteria in a statistically significant manner.

The threshold number can be expressed using any appropriate measure, including without limitation as a number of loci or as a percentage of loci. In some embodiments, the threshold number is less than about 10%, 9%, 8%, 7%, 6%, 5%, 4%, 3%, 2%, 1%, 0.9%, 0.8%, 0.7%, 0.6%, 0.5%, 0.4%, 0.3%, 0.2%, or 0.1% of the number of members of the plurality of microsatellite loci. On the other hand, the threshold number can be greater than about 10%, 9%, 8%, 7%, 6%, 5%, 4%, 3%, 2%, 1%, 0.9%, 0.8%, 0.7%, 0.6%, 0.5%, 0.4%, 0.3%, 0.2%, or 0.1% of the number of members of the plurality of microsatellite loci. For example, the threshold number can be between about 10% and about 0.1% of the number of members of the plurality of microsatellite loci, or between about 5% and about 0.2% of the number of members of the plurality of microsatellite loci, or between about 3% and about 0.3% of the number of members of the plurality of microsatellite loci, or between about 1% and about 0.4% of the number of members of the plurality of microsatellite loci. As used herein, “about” may include a range of +/−10% of the stated value.

As an example of the method of determining MSI, the number of members of the plurality of microsatellite loci is greater than 7000 and the threshold number is ≥40 and ≤50, wherein optionally the threshold level is 40, 41, 42, 43, 44, 45, 46, 47, 48, 49 or 50. Example 8 herein presents one illustration of the method of determining MSJ. In the Example the members of the plurality of microsatellite loci are those in Table 16, which comprises 7317 members. Using the methods described herein, the threshold was set to 46 loci. Accordingly, the threshold was 0.63% of the number of members of the plurality of microsatellite loci. The threshold can be recalibrated as described herein with changing members of the plurality of microsatellite loci.

In preferred embodiments of the method of determining MSI, MSI status, e.g., high, stable or low, is determined without assessing microsatellite loci in normal tissue. Thus, the invention can avoid taking additional tissue from an individual.

In embodiments of the method of determining MSI, the method further comprises identifying the biological sample as microsatellite stable (MSS) if the number of altered microsatellite loci is below the threshold number. Relatedly, the method may also comprise identifying the biological sample as MSI-low if the number of altered microsatellite loci in the sample is less than or equal to a lower threshold number. As further described herein, the MSI-low can be calibrated using similar methodology as MSI high described above. MSS can be the range between MSI-high and MSH-low.

The invention also provides a method of determining a tumor mutation burden (TMB; also referred to as tumor mutation load or TML) for a biological sample. In embodiments of the method of determining MSI, the method further comprises determining a tumor mutation burden (TMB) for the biological sample. In preferred embodiments, TMB is determined using the same laboratory analysis as MSJ. As a non-limiting illustration, a NGS panel is run on a biological sample and the sequencing results are used to calculate MSI, TMB, or both. In some embodiments, TMB is determined by sequence analysis of a plurality of genes, including without limitation cancer genes selected from Table 7, Table 8, Table 9, Table 10, or any combination thereof. In a preferred embodiment, TMB is determined using missense mutations that have not been previously identified as germline alterations in the art. Similar to MSI-high, TMB-High can be determined by comparing a mutation rate to a TMB-High threshold, wherein TMB-High is defined as the mutation rate greater than or equal to the TMB-High threshold. The mutation rate can be expressed in any appropriate units, including without limitation units of mutations/megabase. The TMB-High threshold can be determined by comparing TMB with MSI determined in colorectal cancer from a same sample. This is because TMB and MSI may be more strongly correlated in CRC than in other types of cancer. In various embodiments, the TMB-High threshold is greater than or equal to 5, 6, 7, 8, 9, 10, 11, 12, 13, 14, 15, 16, 17, 18, 19, 20, 21, 22, 23, 24 or 25 mutations/megabase of missense mutations. In a preferred embodiment, the TMB-High threshold is 17 mutations/megabase. Similarly, TMB-Low status can be determined by comparing a mutation rate to a TMB-Low threshold, wherein TMB-Low is defined as the mutation rate less than or equal to the TMB-Low threshold. The TMB-Low threshold can also be determined by comparing TMB with MSI determined in colorectal cancer from a same sample. In various embodiments, the TMB-Low threshold is less than or equal to 15, 14, 13, 12, 11, 10, 9, 8, 7, 6, 5, 4, 3, 2, or 1 mutations/megabase of missense mutations. In a preferred embodiment, the TMB-Low threshold is 6 mutations/megabase.

As with MSI described above, the TMB thresholds can be recalibrated when sequencing results are obtained for different genes or different regions of the same genes. The TMB thresholds can also be recalibrated for different settings wherein sufficient data is available to tune sensitivity, specificity, positive predictive value, negative predictive value, or other criteria in a robust manner.

In embodiments of the method of determining MSI, TMB, or both, the method further comprises profiling various additional biomarkers in the biological sample as desired, e.g., mismatch repair proteins such as MLH1, MSH2, MSH6, and PMS2, immune checkpoint proteins such as PD-L1, or any combination thereof. The profiling can comprise any useful technique, including without limitation determining: i) a protein expression level, wherein optionally the protein expression level is determined using IHC, flow cytometry or an immunoassay; ii) a nucleic acid sequence, wherein optionally the sequence is determined using next generation sequencing; iii) a promoter hypermethylation, wherein optionally the hypermethylation is determined using pyrosequencing; and iv) any combination thereof. For example, it may be desired to profile promoter hypermethylation of MLH1; mutations in MLH1, MSH2, MSH6, and PMS2; protein expression of MLH1, MSH2, MSH6, PMS2 and PD-L1; and any combination thereof. Checkpoint proteins of interest can include PD-1, PD-L1, PD-L2, CTL4A, IDO1, COX2, CD80, CD86, CD8A, Granzyme A, Granzyme B, CD19, CCR7, CD276, LAG-3, TIM-3, or any useful combination thereof.

In another aspect, the invention provides a method of identifying at least one therapy of potential benefit for an individual with cancer, the method comprising: (a) obtaining a biological sample from the individual, e.g., as described herein; (b) generating a molecular profile by performing the method of the invention for determining MSI, TMB, or both on the biological sample (e.g., as described above); and (c) identifying the therapy of potential benefit based on the molecular profile. Generating the molecular profile can also comprise performing additional analysis on the biological sample according to Table 5, Table 6, Table 7, Table 8, Table 9, Table 10, or any combination thereof. In some embodiments, generating the molecular profile comprises performing additional analysis on the biological sample to: i) determine a tumor mutation burden (TMB); ii) determine an expression level of MLH1; iii) determine an expression level of MSH2, determine an expression level of MSH6; iv) determine an expression level of PMS2; v) determine an expression level of PD-L1; vi) or any combination thereof. Additional analysis maybe be useful, e.g., promoter hypermethylation of MLH1; mutations in MLH1, MSH2, MSH6, and PMS2; protein expression of MLH1, MSH2, MSH6, PMS2 and PD-L1; and any combination thereof.

The step of identifying can use drug-biomarker associations, such as those described herein. See, e.g., Table 11. The step of identifying can use drug-biomarker association rule sets such as in any of International Patent Publications WO/2007/137187 (Int'l Appl. No. PCT/US2007/069286), published Nov. 29, 2007; WO/2010/045318 (Int'l Appl. No. PCT/US2009/060630), published Apr. 22, 2010; WO/2010/093465 (Int'l Appl. No. PCT/US2010/000407), published Aug. 19, 2010; WO/2012/170715 (Int'l Appl. No. PCT/US2012/041393), published Dec. 13, 2012; WO/2014/089241 (Int'l Appl. No. PCT/US2013/073184), published Jun. 12, 2014; WO/2011/056688 (Int'l Appl. No. PCT/US2010/054366), published May 12, 2011; WO/2012/092336 (Int'l Appl. No. PCT/US2011/067527), published Jul. 5, 2012; WO/2015/116868 (Int'l Appl. No. PCT/US2015/013618), published Aug. 6, 2015; WO/2017/053915 (Int'l Appl. No. PCT/US2016/053614), published Mar. 30, 2017; and WO/2016/141169 (Int'l Appl. No. PCT/US2016/020657), published Sep. 9, 2016; each of which publications is incorporated by reference herein in its entirety. In a preferred embodiment, the step of identifying comprises identifying potential benefit from an immune checkpoint inhibitor therapy when the biological sample is MSI-High. Similarly, the step of identifying may comprise identifying potential benefit from an immune checkpoint inhibitor therapy when the biological sample is MSI-High, TMB-High, MLH1-, MSH2-, MSH6-, PMS2-, PD-L1+, or any combination thereof. The step of identifying may comprise identifying potential benefit from an immune checkpoint inhibitor therapy when the biological sample is MSI-High, TMB-High, PD-L1+, or any combination thereof. See, e.g., Example 8 herein, which notes that each of these biomarkers can provide independent information; see also FIGS. 27A-BR and related text. The method can identify any useful immune checkpoint inhibitor therapy, including without limitation ipilimumab, nivolumab, pembrolizumab, atezolizumab, avelumab, durvalumab, pidilizumab, AMP-224, AMP-514, PDR001, BMS-936559, or any combination thereof. In addition, the method may comprise identifying at least one therapy of potential lack of benefit based on the molecular profile, at least one clinical trial for the subject based on the molecular profile, or any combination thereof. For examples, see FIGS. 27A-BR.

In embodiments of the method of identifying at least one therapy of potential benefit, the subject has not previously been treated with the at least one therapy of potential benefit. The cancer may comprise a metastatic cancer, a recurrent cancer, or any combination thereof. In some cases, the cancer is refractory to a prior therapy, including without limitation front-line or standard of care therapy for the cancer. In some embodiments, the cancer is refractory to all known standard of care therapies. In other embodiments, the subject has not previously been treated for the cancer. The method may further comprise administering the at least one therapy of potential benefit to the individual. Progression free survival (PFS), disease free survival (DFS), or lifespan can be extended by the administration.

The method of identifying at least one therapy of potential benefit can be employed for any desired cancer, such as those disclosed herein. In some embodiments, the cancer is of a lineage listed in Table 19.

In a related aspect, the invention provides a method of generating a molecular profiling report comprising preparing a report comprising the generated molecular profile using the methods of the invention above. In some embodiments, the report further comprises a list of the at least one therapy of potential benefit for the individual. In some embodiments, the report further comprises a list of at least one therapy of potential lack of benefit for the individual. In some embodiments, the report further comprises a list of at least one therapy of indeterminate benefit for the individual. The report may comprise identification of the at least one therapy as standard of care or not for the cancer lineage. The report can also comprise a listing of biomarkers tested when generating the molecular profile, the type of testing performed for each biomarker, and results of the testing for each biomarker. In some embodiments, the report further comprises a list of clinical trials for which the subject is indicated and/or eligible based on the molecular profile. In some embodiments, the report further comprises a list of evidence supporting the identification of therapies as of potential benefit, potential lack of benefit, or indeterminate benefit based on the molecular profile. The report can comprise any or all of these elements. For example, the report may comprise: 1) a list of biomarkers tested in the molecular profile; 2) a description of the molecular profile of the biomarkers as determined for the subject (e.g., type of testing and result for each biomarker); 3) a therapy associated with at least one of the biomarkers in the molecular profile; and 4) and an indication whether each therapy is of potential benefit, potential lack of benefit, or indeterminate benefit for treating the individual based on the molecular profile. The description of the molecular profile of the biomarkers can include the technique used to assess the biomarkers and the results of the assessment. The report can be computer generated, and can be a printed report, a computer file or both. The report can be made accessible via a secure web portal.

In an aspect, the invention provides the report generated by the methods of the invention. In a related aspect, the invention provides a computer system for generating the report. Exemplary reports generated according to the methods of the invention, and generated by a system of the invention, are found herein in FIGS. 27A-BR. See also Example 3.

In an aspect, the invention provides use of a reagent in carrying out the methods of the invention as described above. In a related aspect, the invention provides of a reagent in the manufacture of a reagent or kit for carrying out the methods of the invention as described above. In still another related aspect, the invention provides a kit comprising a reagent for carrying out the methods of the invention as described above. The reagent can be any useful and desired reagent. In preferred embodiments, the reagent comprises at least one of a reagent for extracting nucleic acid from a sample, a reagent for performing ISH, a reagent for performing IHC, a reagent for performing PCR, a reagent for performing Sanger sequencing, a reagent for performing next generation sequencing, a probe set for performing next generation sequencing, a probe set for sequencing the plurality of microsatellite loci, a reagent for a DNA microarray, a reagent for performing pyrosequencing, a nucleic acid probe, a nucleic acid primer, an antibody, an aptamer, a reagent for performing bisulfite treatment of nucleic acid, and any combination thereof.

In an aspect, the invention provides a system for identifying at least one therapy associated with a cancer in an individual, comprising: (a) at least one host server; (b) at least one user interface for accessing the at least one host server to access and input data; (c) at least one processor for processing the inputted data; (d) at least one memory coupled to the processor for storing the processed data and instructions for: i) accessing an MSI status generated by the method of the invention above; and ii) identifying, based on the MSI status, at least one of: A) at least one therapy with potential benefit for treatment of the cancer; B) at least one therapy with potential lack of benefit for treatment of the cancer; and C) at least one therapy associated with a clinical trial; and (e) at least one display for displaying the identified at least one of: A) at least one therapy with potential benefit for treatment of the cancer; B) at least one therapy with potential lack of benefit for treatment of the cancer; and C) at least one therapy associated with a clinical trial. In some embodiments, the system further comprises at least one memory coupled to the processor for storing the processed data and instructions for identifying, based on the generated molecular profile according to the methods above, at least one of: A) at least one therapy with potential benefit for treatment of the cancer; B) at least one therapy with potential lack of benefit for treatment of the cancer; and C) at least one therapy associated with a clinical trial; and at least one display for display thereof. The system may further comprise at least one database comprising references for various biomarker states, data for drug/biomarker associations, or both. The at least one display can be a report provided by the invention. See, e.g., the report herein in FIGS. 27A-BR. See also Example 3.

### Examples

### Example 1: Molecular Profiling System

Molecular profiling is performed to determine a treatment for a disease, typically a cancer. Using a molecular profiling approach, molecular characteristics of the disease itself are assessed to determine a candidate treatment. Thus, this approach provides the ability to select treatments without regard to the anatomical origin of the diseased tissue, or other “one-size-fits-all” approaches that do not take into account personalized characteristics of a particular patient's affliction. The profiling comprises determining gene and gene product expression levels, gene copy number and mutation analysis. Treatments are identified that are indicated to be effective against diseased cells that overexpress certain genes or gene products, underexpress certain genes or gene products, carry certain chromosomal aberrations or mutations in certain genes, or any other measureable cellular alterations as compared to non-diseased cells. Because molecular profiling is not limited to choosing amongst therapeutics intended to treat specific diseases, the system has the power to take advantage of any useful technique to measure any biological characteristic that can be linked to a therapeutic efficacy. The end result allows caregivers to expand the range of therapies available to treat patients, thereby providing the potential for longer life span and/or quality of life than traditional “one-size-fits-all” approaches to selecting treatment regimens.

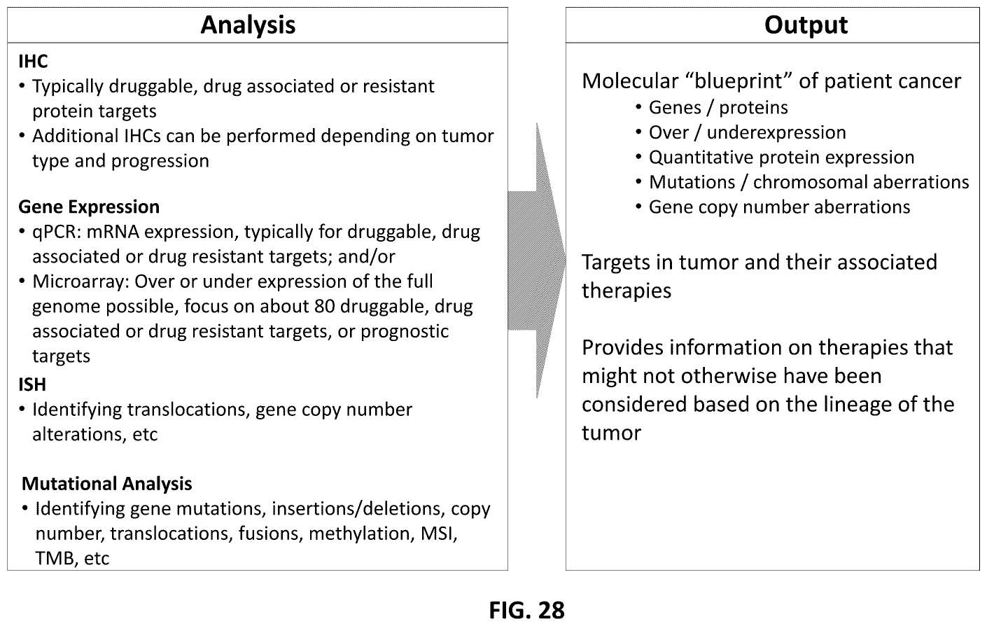

FIG. 28 illustrates a molecular profiling system that performs analysis of a cancer sample using a variety of components that measure various biological aspects including without limitation expression levels, chromosomal aberrations and mutations. The molecular “blueprint” of the cancer is used to generate a prioritized ranking of druggable targets and/or drug associated targets in tumor and their associated therapies.

A system for carrying out molecular profiling according to the invention comprises the components used to perform molecular profiling on a patient sample, identify potentially beneficial and non-beneficial treatment options based on the molecular profiling, and return a report comprising the results of the analysis to the treating physician or other appropriate caregiver.

Formalin-fixed paraffin-embedded (FFPE) can be reviewed by a pathologist for quality control before subsequent analysis. Nucleic acids (DNA and RNA) can be extracted from FFPE tissues after microdissection of the fixed slides. Nucleic acids can be extracted using methods such as phenol-chlorform extraction or kits such as the QIAamp DNA FFPE Tissue kit according to the manufacturer's instructions (QIAGEN Inc., Valencia, CA).

Gene expression analysis can be performed using an expression microarray or qPCR (RT-PCR). The qPCR can be performed using a low density microarray. In addition to gene expression analysis, the system can perform a set of immunohistochemistry assays on the input sample. Gene copy number is determined for a number of genes via ISH (in situ hybridization) and mutational analysis can be performed by DNA sequencing (including sequence sensitive PCR assays and fragment analysis such as RFLP, as desired) for specific mutations. Comprehensive sequencing analysis with high throughput techniques (also known as next generation sequencing, NGS) can be performed to assess numerous genes, including whole exome analysis, and numerous types of alterations in high throughput fashion. For example, NGS can be used to assess mutations, including point mutations, insertions, deletions, and copy number in DNA, and gene fusions and copy number in RNA. Molecular profiling data can be stored for each patient case. Data is reported from any desired combination of analysis performed. All laboratory experiments are performed according to Standard Operating Procedures (SOPs).

Expression can be measured using real-time PCR (qPCR, RT-PCR). The analysis can employ a low density microarray. The low density microarray can be a PCR-based microarray, such as a Tagman™ Low Density Microarray (Applied Biosystems, Foster City, CA).

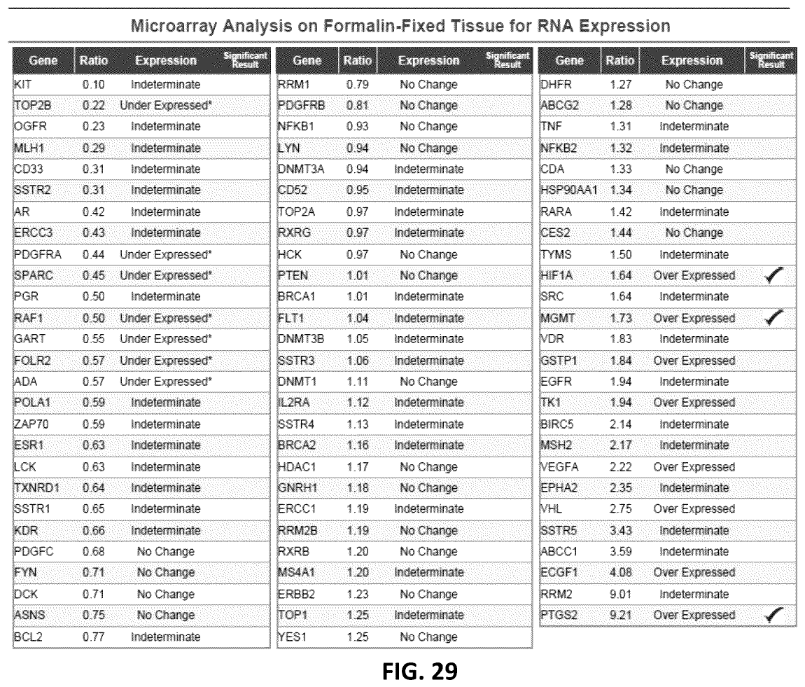

Expression can be measured using a microarray. The expression microarray can be an Agilent 44K chip (Agilent Technologies, Inc., Santa Clara, CA). This system is capable of determining the relative expression level of roughly 44,000 different sequences through RT-PCR from RNA extracted from fresh frozen tissue. Alternately, the system uses the Illumina Whole Genome DASL assay (Illumina Inc., San Diego, CA), which offers a method to simultaneously profile over 24,000 transcripts from minimal RNA input, from both fresh frozen (FF) and formalin-fixed paraffin embedded (FFPE) tissue sources, in a high throughput fashion. The analysis makes use of the Whole-Genome DASL Assay with UDG (Illumina, cat #DA-903-1024/DA-903-1096), the Illumina Hybridization Oven, and the Illumina iScan System according to the manufacturer's protocols. FIG. 29 shows example results obtained from microarray profiling of an FFPE sample. Total RNA was extracted from tumor tissue and was converted to cDNA. The cDNA sample was then subjected to a whole genome (24K) microarray analysis using the Illumina Whole Genome DASL process. The expression of a subset of 80 genes was then compared to a tissue specific normal control and the relative expression ratios of these 80 target genes indicated in the figure was determined as well as the statistical significance of the differential expression.

Polymerase chain reaction (PCR) amplification is performed using the ABI Veriti Thermal Cycler (Applied Biosystems, cat #9902). PCR is performed using the Platinum Taq Polymerase High Fidelity Kit (Invitrogen, cat #11304-029). Amplified products can be purified prior to further analysis with Sanger sequencing, pyrosequencing or the like. Purification is performed using CleanSEQ reagent, (Beckman Coulter, cat #000121), AMPure XP reagent (Beckman Coulter, cat #A63881) or similar. Sequencing of amplified DNA is performed using Applied Biosystem's ABI Prism 3730x1 DNA Analyzer and BigDye® Terminator V1.1 chemistry (Life Technologies Corporation, Carlsbad, CA). The BRAF V600E mutation is assessed using the FDA approved cobas® 4800 BRAF V600 Mutation Test from Roche Molecular Diagnostics (Roche Diagnostics, Indianapolis, IN). NextGeneration sequencing is performed using the MiSeq platform from Illumina Corporation (San Diego, California, USA) according to the manufacturer's recommended protocols.

For RFLP, fragment analysis can performed on reverse transcribed mRNA isolated from a formalin-fixed paraffin-embedded tumor sample using FAM-linked primers designed to flank and amplify desired locations.

IHC is performed according to standard protocols. IHC detection systems vary by marker and include Dako's Autostainer Plus (Dako North America, Inc., Carpinteria, CA), Ventana Medical Systems Benchmark® XT (Ventana Medical Systems, Tucson, AZ), and the Leica/Vision Biosystems Bond System (Leica Microsystems Inc., Bannockburn, IL). All systems are operated according to the manufacturers' instructions.

ISH is performed on formalin-fixed paraffin-embedded (FFPE) tissue. FFPE tissue slides for FISH must be Hematoxylin and Eosion (H & E) stained and given to a pathologist for evaluation. Pathologists will mark areas of tumor to be ISHed for analysis. The pathologist report must show tumor is present and sufficient enough to perform a complete analysis. FISH or CISH are performed using the Abbott Molecular VP2000 according to the manufacturer's instructions (Abbott Laboratories, Des Plaines, IA). ALK can be assessed using the Vysis ALK Break Apart FISH Probe Kit from Abbott Molecular, Inc. (Des Plaines, IL). HER2 can be assessed using the INFORM HER2 Dual ISH DNA Probe Cocktail kit from Ventana Medical Systems, Inc. (Tucson, AZ) and/or SPoT-Light® HER2 CISH Kit available from Life Technologies (Carlsbad, CA).

DNA for mutation analysis is extracted from formalin-fixed paraffin-embedded (FFPE) tissues after macrodissection of the fixed slides in an area that % tumor nuclei ≥10% as determined by a pathologist. Extracted DNA is only used for mutation analysis if % tumor nuclei ≥10%. DNA is extracted using the QIAamp DNA FFPE Tissue kit according to the manufacturer's instructions (QIAGEN Inc., Valencia, CA). DNA can also be extracted using the fQuickExtract™ FFPE DNA Extraction Kit according to the manufacturer's instructions (Epicentre Biotechnologies, Madison, WI). The BRAF Mutector I BRAF Kit (TrimGen, cat #MH1001-04) is used to detect BRAF mutations (TrimGen Corporation, Sparks, MD). Roche's Cobas PCR kit can be used to assess the BRAF V600E mutation. The DxS KRAS Mutation Test Kit (DxS, #KR-03) is used to detect KRAS mutations (QIAGEN Inc., Valencia, CA). BRAF and KRAS sequencing of amplified DNA is performed using Applied Biosystems' BigDye® Terminator V1.1 chemistry (Life Technologies Corporation, Carlsbad, CA).

Next generation sequencing is performed using a TruSeq/MiSeq/HiSeq/NexSeq system offered by Illumina Corporation (San Diego, CA) or an Ion Torrent system from Life Technologies (Carlsbad, CA, a division of Thermo Fisher Scientific Inc.) according to the manufacturer's instructions.

### Example 2: Molecular Profiling Service

FIGS. 26A-C illustrate a molecular profiling service requisition using a molecular profiling approach as outlined in Tables 5-11, and accompanying text herein. Such requisition presents choices for molecular profiling that can be presented to a caregiver, e.g., a medical oncologist who may prescribe a therapeutic regimen to a cancer patient. FIG. 26A shows a choice of MI Profile™ panel that is assessed using multiple technologies, e.g., according to Table 5 (which, as noted, preferably comprises Tables 6-10 for NGS), or a MI Tumor Seek™ panel, e.g., with the gene analysis presented in Tables 6-10. FIG. 26B amd FIG. 26C illustrate sample requirements that can be used to perform molecular profiling on a patient tumor sample according to the biomarker choices in FIG. 26A. FIG. 26B provides requirements for formalin fixed paraffin embedded (FFPE) and FIG. 26C provides requirements for fresh samples or insufficient sample to perform all testing. In the event that insufficient quantity or tissue, bodily fluid or percent tumor is available to perform all tests desired to be performed, certain tests can be prioritized, e.g., according to physician preference or experience with the various biomarkers in similar tumor types.

FIGS. 26D-E illustrate sample requirements and corresponding test performance. FIG. 26D shows expected technical sensitivity and specificity of ISH, CISH and FISH. FIG. 26E shows expected technical criteria including positive predictive value (PPV), sensitivity and specificity of Next Generation Sequencing (NGS).

Using the comprehensive genomic profiling approach provided herein to assess DNA, RNA and proteins reveals a reliable molecular blueprint to guide more precise and individualized treatment decisions from among 60+FDA-approved therapies (at present).

### Example 3: Molecular Profiling Reports

FIGS. 27A-BR present molecular profiling reports of the invention which are de-identified but from molecular profiling of actual patients according to the systems and methods of the invention.

FIGS. 27A-Z illustrate an exemplary patient report based on molecular profiling the tumor of an individual having breast cancer. FIG. 27A illustrates a cover page of a report indicating patient and specimen information for the patient. Note that the molecular profiling results indicate ER/PR positive and HER2 negative under the header “Lineage Relevant Biomarkers.” Under the header “Other Notable Biomarker Results,” note that the patient is considered both TMB (“Tumor Mutation Load”) high (49 Mutations/Mb) and MSI high. FIG. 27A also displays a summary of therapies associated with potential benefit, therapies associated with uncertain benefit, and therapies associated with potential lack of benefit. These sections indicate the relevant biomarkers for the therapeutic associations. Agents associated with potential benefit are highlighted in bold if the drug/biomarker association(s) are supported by the highest level of clinical evidence. For this patient, the MSI results suggested potential benefit of the anti PD-1 antibody pembrolizumab. The lack of HER2 suggested potential lack of benefit from anti-HER2 therapies. FIG. 27B continues from FIG. 27A. FIGS. 27C-D provide a summary of biomarker results from the indicated assays. The biomarkers comprise those most commonly associated with cancer. Further results for additional biomarkers are described in the appendix. FIG. 27E provides a number of significant notes for the ordering physician, e.g., a note concerning clinical trials in the appendix, and details about the patient sample and analyses performed on the sample. FIGS. 27F-I provide additional information about drug recommendations shown on the first pages. These sections indicate whether the associations are FDA-approved or ON-NCCN COMPENDIUM®, or OFF-NCCN COMPENDIUM®. FIGS. 27F-G provide more detailed information for biomarker profiling used to associate agents with potential benefit. As noted on the front page, agents associated with potential benefit are highlighted in bold if the drug/biomarker association(s) are supported by the highest level of clinical evidence. For example, the OFF-NCCN COMPENDIUM® section notes that nivolumab and pembrolizumab are associated with potential benefit for treating the patient's breast cancer because the sample was determined to be MSI high based on analysis with NGS. FIG. 27H illustrates more detailed information for biomarker profiling used to associate agents with uncertain benefit. The report notes that therapies are placed in the uncertain benefit category when a result suggests only a decreased likelihood of response (vs. little to no likelihood of response) or if there is insufficient evidence to associate the drug with either benefit or lack of benefit. The appendix to the report will provide further information about the results and why the association was made. FIG. 271 illustrates more detailed information for biomarker profiling used to associate agents with lack of potential benefit. FIG. 27J provides information for biomarker profiling matched to potential clinical trials for which the patient might be enrolled. The page notes that additional information pertaining to clinical trials relevant to the patient are made available to the ordering physician over a web portal (“MI Portal”). As noted, the patient may be matched to multiple trials for a given biomarker result. FIG. 27K presents a disclaimer, noting, inter alia, that “[t]he decision to select any, all, or none of the listed therapies resides within the discretion of the treating physician.” The remainder of the report comprises an appendix with additional details about the molecular profiling that was performed and evidence used to make drug-treatment associations. FIGS. 27L-27T provide more details about results obtained through NGS analysis. FIG. 27L provides information about the TMB analysis and results. The report notes that high mutational load is a potential indicator of immunotherapy response (Le et al., PD-1 Blockade in Tumors with Mismatch-Repair Deficiency, N Engl J Med 2015; 372:2509-2520; Rizvi et al., Mutational landscape determines sensitivity to PD-1 blockade in non-small cell lung cancer. Science. 2015 Apr. 3; 348(6230): 124-128; Rosenberg et al., Atezolizumab in patients with locally advanced and metastatic urothelial carcinoma who have progressed following treatment with platinum-based chemotherapy: a single arm, phase 2 trial. Lancet. 2016 May 7; 387(10031): 1909-1920; Snyder et al., Genetic Basis for Clinical Response to CTLA-4 Blockade in Melanoma. N Engl J Med. 2014 Dec. 4; 371(23): 2189-2199; all of which references are incorporated by reference herein in their entirety). FIGS. 27L-270 list details concerning the genes found to harbor alterations. As shown, this patient had a high TMB and alterations were found in a number of genes. FIG. 27P notes genes that were tested by NGS with no detected alterations. FIG. 27Q summarizes genes tested that were found to have unclassified mutations, e.g., these mutations have not previously been identified as pathogenic, and also lists genes with indeterminate results, e.g., due to low coverage for some or all exons during the NGS runs. FIG. 27R provides more information about how Next Generation Sequencing was performed. FIG. 27S provides information about gene amplification (“CNV” or copy number variation) detected by NGS analysis and corresponding methodology. FIG. 27T provides information about MSI detected by NGS analysis and corresponding methodology. As noted, this patient was considered MSI high based on the NGS results. FIG. 27U provides more information about the IHC analysis performed on the patient sample, e.g., the staining threshold and results for each marker. FIG. 27V provides more information about the ISH analysis performed on the patient sample, which comprised CISH for TOP2A. FIG. 27W, FIG. 27X, and FIG. 27Y provide a listing of published references used to provide evidence of the biomarker—agent association rules used to construct the therapy recommendations. FIG. 27Z provides the framework used for the literature level of evidence as included in the report.

FIGS. 27AA-AV illustrate an molecular patient report based on molecular profiling the tumor of an individual having colorectal cancer, specifically adenocarcinoma of the cecum. The report follows the same general format as the report above but is tailored to molecular profiling results obtained for this specific patient. FIG. 27AA is the cover page for this report. Under the “Lineage Relevant Biomarkers” section, note that the patient is considered MSI high by NGS. In addition, this patient was found to be negative for expression of the mismatch repair proteins MLH1 and PMS2. These MSI, MLH1 and PMS2 results all point to potential clinical benefit of the anti-PD-1 monoclonal antibodies nivolumab and pembrolizumab based on the highest level of clinical evidence. As shown in the section “Other Notable Biomarker Results,” the tumor was also TMB high (34 Mutations/Mb) and PD-L1 positive as determined by IHC. Unlike the breast cancer patient above, molecular profiling of this patient did not identify any therapies with potential lack of benefit. FIGS. 27AB-AC provide a summary of biomarker results from the indicated assays for the biomarkers most commonly associated with cancer. FIG. 27AD provides a number of significant notes for the ordering physician, e.g., a note concerning clinical trials in the appendix, and details about the patient sample and analyses performed on the sample. For this case, the notes also explain that the tumor displays evidence of MMR protein deficiency and recommends testing for Lynch Syndrome. FIG. 27AE provides more detailed information for biomarker profiling used to associate agents with potential benefit. FIG. 27AF illustrates more detailed information for biomarker profiling used to associate agents with uncertain benefit. FIGS. 27AG-AH provide information for biomarker profiling matched to potential clinical trials for which the patient might be enrolled. FIG. 27AI presents a disclaimer, noting, inter alia, that “[t]he decision to select any, all, or none of the listed therapies resides within the discretion of the treating physician.” The remainder of the report comprises an appendix with additional details about the molecular profiling that was performed and evidence used to make drug-treatment associations. FIGS. 27AJ-27AR provide more details about results obtained through NGS analysis. FIG. 27AJ provides information about the TMB analysis and results. FIGS. 27AJ-27AN list details concerning the genes found to harbor alterations. FIG. 27AN also notes genes that were tested by NGS with no mutations detected. FIG. 27AO summarizes genes tested that were found to have unclassified mutations, e.g., these mutations have not previously been identified as pathogenic, and also lists genes with indeterminate results, e.g., due to low coverage for some or all exons during the NGS runs. FIG. 27AP provides more information about how Next Generation Sequencing was performed. FIG. 27AQ provides information about gene amplification (“CNV” or copy number variation) detected by NGS analysis and corresponding methodology. Unlike the breast cancer case in the report above, no CNSs were detected for this CRC patient. FIG. 27AR provides information about MSI detected by NGS analysis and corresponding methodology. As noted, this patient was considered MSI high based on the NGS results. FIG. 27AS provides more information about the IHC analysis performed on the patient sample, e.g., the staining threshold and results for each marker. FIG. 27AT and FIG. 27AU provide a listing of published references used to provide evidence of the biomarker—agent association rules used to construct the therapy recommendations. FIG. 27AV provides the framework used for the literature level of evidence as included in the report.

FIGS. 27AW-BR illustrate an exemplary patient report based on molecular profiling the tumor of an individual having a non-small cell carcinoma of the lung (NSCLC). The report follows the same general format as the reports above but is tailored to molecular profiling results obtained for this specific patient. FIG. 27AW and FIG. 27AX are the cover page for this report. Under the “Lineage Relevant Biomarkers” section, note that fusions were not detected via RNA sequencing in the ROS1 or RET genes. The patient's tumor was found to have high expression of PD-L1 by IHC, suggesting potential benefit of the anti-PD-1 monoclonal antibodies nivolumab and pembrolizumab and the anti-PD-L1 monoclonal antibody atezolizumab, each based on the highest level of clinical evidence. As shown in the section “Other Notable Biomarker Results,” the tumor was also TMB high (36 Mutations/Mb) but MSI stable. Thus, PD-L1 and TMB but not MSI would suggest immune checkpoint therapies for this patient. The cover page lists several therapies with potential benefit for treating the patient and several therapies with potential lack of benefit for treating the patient. The molecular profiling did not identify therapies with uncertain benefit. FIG. 27AX continues from FIG. 27AW. This section notes that the PD-L1 result is sufficient to guide pembrolizumab use for front-line, metastatic & pretreated, metastatic NSCLC, but that nivolumab & atezolizumab are not FDA-approved in the front-line, metastatic setting. FIGS. 27AY-AZ provide a summary of biomarker results from the indicated assays. The biomarkers comprise those most commonly associated with cancer. On FIG. 27AZ, the report lists a number of genes tested for RNA alterations by NGS. No fusions or variant transcripts were detected. FIG. 27BA provides a number of significant notes for the ordering physician, e.g., a note concerning clinical trials in the appendix, and details about the patient sample and analyses performed on the sample. FIG. 27BB provides more detailed information for biomarker profiling used to associate agents with potential benefit. For example, the FDA-APPROVED/ON-NCCN COMPENDIUM® section notes that atezolizumab, nivolumab and pembrolizumab are associated with potential benefit for treating the patient's lung cancer because the sample was determined to have high expression of PD-L1 protein by IHC even though the tumor was MSI stable based on analysis with NGS. Again the report points to different approvals for these therapies in this setting. FIG. 27BC and FIG. 27BD illustrate more detailed information for biomarker profiling used to associate agents with lack of potential benefit. FIG. 27BE provides information for biomarker profiling matched to potential clinical trials for which the patient might be enrolled. FIG. 27BF presents a disclaimer, noting, inter alia, that “[t]he decision to select any, all, or none of the listed therapies resides within the discretion of the treating physician.” The remainder of the report comprises an appendix with additional details about the molecular profiling that was performed and evidence used to make drug-treatment associations. FIGS. 27BG-27BM provide more details about results obtained through NGS analysis. FIG. 27BG provides information about the TMB analysis and results. FIG. 27BG also lists details concerning the genes found to harbor alterations. As shown, this patient had a high TMB and pathogenic alterations were found in three genes (KRAS, PBRM1 and TP53). FIG. 27BH notes genes that were tested by NGS with no detected alterations. FIG. 27BI summarizes genes tested that were found to have unclassified mutations, e.g., these mutations have not previously been identified as pathogenic, and also lists genes with indeterminate results, e.g., due to low coverage for some or all exons during the NGS runs. FIG. 27BJ provides more information about how Next Generation Sequencing was performed. FIG. 27BK provides information about gene amplification (“CNV” or copy number variation) detected by NGS analysis and corresponding methodology. In this case, amplification of the FLCN gene was observed but was not evaluated for clinical significance. FIG. 27BL provides information about gene fusion and variant transcript testing that was performed by NGS analysis of RNA. FIG. 27BM provides information about MSI detected by NGS analysis and corresponding methodology. As noted, no MSI was observed based on the NGS results. FIG. 27BN provides more information about the IHC analysis performed on the patient sample, e.g., the staining threshold and results for each marker. FIG. 27BO, FIG. 27BP, and FIG. 27BQ provide a listing of published references used to provide evidence of the biomarker—agent association rules used to construct the therapy recommendations. FIG. 27BR provides the framework used for the literature level of evidence as included in the report.

### Example 4: Molecular Profiling of Immune Checkpoint Related Genes

Clinical response to immune checkpoint inhibitor therapy ranges from 18% to 28% by tumor type. There is unmet clinical need for laboratory tests that can identify patients likely to respond to such therapy. Reports indicate that 36% of transgenic tumors with PD-1 expression responded to anti-PD1 therapy while no PD-1 negative cases responded. Estimated objective responses for tumors expressing FoxP3 and IDO by IHC were 10.38 and 8.72 respectively. This Example used microarray expression data to characterize the presence of immune response modulators in human tumors and possibly identify a subset of cases as the candidates for immune checkpoint inhibitor therapy.

A retrospective analysis of gene expression microarray data for immune related genes was performed on 9,025 qualifying paraffin embedded human tumor specimens (HumanHT-12 v4 beadChip Illumina Inc., San Diego, CA). Samples from LN metastases were excluded from analysis. Immune checkpoint-related genes examined included CTLA4, its binding partners CD80 and CD86, PD-L1, CD276 (B7-H3), Granzymes A and B, CD8a, CD19 and the chemokine receptor CCR7. The normalized expression values for these genes were plotted by tumor types to compare relative expression levels and Principal Component Analysis was performed.

The results of this analysis showed that PD-L1 expression was above the 90th percentile of normal control tissue in 4% of breast cancers, 3% of renal cancers, 7% of NSCLC, 3% ovarian cancer and 5% of colon cancer tumors. Principal component analysis of the immune checkpoint-related genes showed the greatest percentage of “distinct” cases within ovarian, melanoma, colon, gastric and pancreatic cancers.

Microarray analysis can identify tumors with unique immune components that are more likely to respond to immune checkpoint therapy.

### Example 5: PD1 and PDL1 in HPV+ and HPV-/TP53 Mutated Head and Neck Squamous Cell Carcinomas

This Example investigated the role of the programmed death 1 (PD1) and programmed death ligand 1 (PDL1) immunomodulatory axis in head and neck squamous cell carcinoma (HNSCC), a cancer with viral and non-viral etiologies. Determination of the impact of this testing in human papilloma virus (HPV)-positive and HPV-negative/TP53-mutated HNSCC carries great importance due to the development of new immunomodulatory agents.

Thirty-four HNSCC cases, including 16 HPV+ and 18 HPV-/TP53 mutant, were analyzed for the PD1/PDL1 immunomodulatory axis by immunohistochemical methods. HNSCC arising in the following anatomic sites were assessed: pharynx, larynx, mouth, parotid gland, paranasal sinuses, tongue and metastatic SCC consistent with head and neck primary.

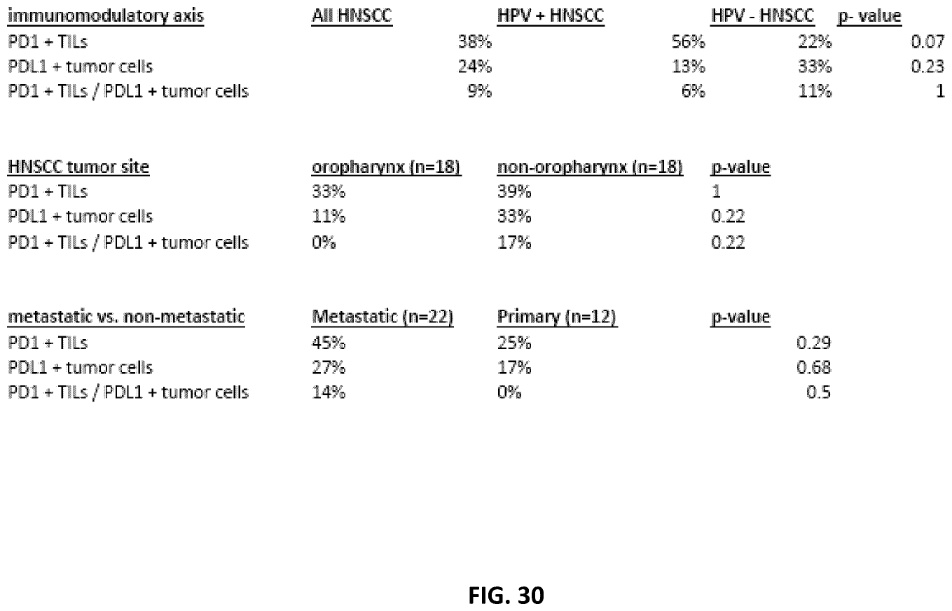

Results are summarized in FIG. 30. 8/34 (24%) HNSCC were positive for cancer cells expression of PDL1, and 13/34 (38%) HNSCC were positive for PD1+tumor infiltrating lymphocytes (TILs). 3/34 (8.8%) were positive for both components of the PD1/PDL1 axis. Comparison of PD1 and PDL1 expression in HPV+ and HPV-/TP53mutant HNSCC showed PD1+TILs were more frequent in HPV+vs. HPV-HNSCC (56% vs. 22%; p=0.07), whereas PDL1+tumor cells more frequent in HPV-vs. HPV+HNSCC (38% vs. 13%; p=0.14). PD1 and PDL1 were expressed in both oropharyngeal and non-oropharyngeal HNSCC: 33% vs. 39% for PD1+TILs, respectively, and 11% and 33% for PDL-1, respectively. To examine the role of PD1 and PDL1 in progression of disease, expression was compared between metastatic and non-metastatic HNSCC. PD1+TILs were detected in 45% of metastatic vs. 25% non-metastatic HNSCC (p=0.29), and PDL1 was detected in 27% vs. 17% of metastatic vs. non-metastatic HNSCC. Interestingly, the three cases that were positive for both PD1 and PDL1 were metastatic HNSCC, including a tumor of the mandible which had metastasized to the bone of the arm, and two unknown primary consistent with head and neck primary, one metastatasized to the lymph nodes and the other metastasized to the lung.

Immune evasion through the PD1/PDL1 axis is relevant to both viral (HPV) and non-viral (TP53) etiologies of HNSCC. Expression of both axis components was less frequently observed across HNSCC tumor sites, and elevated expression of both PD1 and PLD1 was seen at a higher frequency in metastatic HNSCC. In summary, we observed that: 1) PDL1+TILs were more frequent (56%) in HPV+HNSCC; 2) PD1 expression was more frequent (38%) in HPV-/TP53 mutated HNSCC; 3) elevation of both components of the axis (PD1 and PDL1), occurs at low frequency (8%); 4) expression of PDL1 and PD1 occurs in head and neck cancers that occur in oropharyngeal and non-oropharyngeal sites; and 5) the PD1/PDL1 pathway is more frequently expressed in metastatic cases vs. non-metastatic HNSCC.

### Example 6: Mutations on the Homologous Recombination (HR) Pathway in 13 Cancer Types

Background: HR pathway is important in DNA double strand break repair. Defects of HR promote carcinogenesis and are associated with selective sensitivity to PARPi and DNA-damaging agents including platinum. We used next-generation sequencing (NGS) to survey genes on the HR pathway in 1029 tumors in 13 cancer types.

Method: NGS on ˜600 whole genes (see Tables 6-10) was performed using formalin-fixed paraffin-embedded samples on the Illumina NextSeq platform. All variants were detected with >99% confidence and with the sensitivity of 10%. Variants that are pathogenic or presumed pathogenic are counted as mutations.

Results: Table 13 summarizes mutation rates of 7 key genes (ATM, BRCA1, BRCA2, CHEK1, CHEK2, PALB2 and PTEN) included in this study. PTEN mutations were seen in 6.3% of tumors, ATM in 5%, BRCA1 in 2%, BRCA2 in 2%, PALB2 in 1%, CHEK2 in 1% and CHEKI mutation is not seen in the cohort studied. Overall, 15% of tumors carry at least one mutation in any of the 7 genes, and the highest mutation rates were seen in endometrial (43%), GBM (34%) and gastric cancers (23%). The highest rates of ATM (9.7%), BRCA2 (6.5%) and PALB2 (6.5%) were seen in gastric cancer while the highest CHEK2 (5.6%), BRCA1 (7.3%) and PTEN (44%) mutations were seen in cholangiocarcinoma, ovarian and endometrial tumors, respectively.

Exceptional response was seen in a 53-year old patient with metastatic poorly-differentiated adenocarcinoma of the stomach after 4 cycles of FOLFOX without surgery, which included ongoing radiographic partial response and dramatic relief of symptoms. A nonsense mutation on PALB2 (S326*) was found while the other 23 HRD genes were wild type; ERCC1 IHC showed intact expression.

TABLE 13
Mutation rates of 7 key genes

| | Biomarker | | | | | | | |
|---|---|---|---|---|---|---|---|---|
| Tumor type | ATM | BRCA1 | BRCA2 | CHEK1 | CHEK2 | PALB2 | PTEN | Any of 7 |
| Endometrial (N = 35) | 0 | 0 | 0 | 0 | 2.9% | 3.0% | 44.1% | 42.9% |
| GBM (N = 47) | 2.1% | 2.1% | 0 | 0 | 0 | 0 | 30.4% | 34.0% |
| Gastric (N = 31) | 9.7% | 0 | 6.5% | 0 | 0 | 6.5% | 0 | 22.6% |
| Bladder (N = 38) | 2.6% | 0 | 5.4% | 0 | 0 | 0 | 10.8% | 18.4% |
| Kidney (N = 41) | 2.5% | 0 | 0 | 0 | 5.0% | 0 | 10.0% | 17.1% |
| Ovarian (N = 82) | 3.7% | 7.3% | 1.2% | 0 | 1.2% | 0 | 1.3% | 14.6% |
| Breast (N = 108) | 4.6% | 2.8% | 1.9% | 0 | 0.9% | 1.0% | 3.8% | 13.9% |
| Cholangiocarcinoma | 2.8% | 0 | 2.8% | 0 | 5.6% | 0 | 2.9% | 13.9% |
| (N = 36) | | | | | | | | |
| CRC (N = 254) | 6.3% | 2.0% | 1.6% | 0 | 0.4% | 0 | 4.0% | 13.0% |
| Pancreatic (N = 62) | 4.8% | 1.6% | 3.2% | 0 | 0 | 1.7% | 3.3% | 12.9% |
| NSCLC (N = 234) | 6.5% | 0 | 0.9% | 0 | 0 | 1.4% | 2.6% | 11.1% |
| Neuroendocrine | 2.9% | 0 | 0 | 0 | 0 | 0 | 5.7% | 8.6% |
| (N = 35) | | | | | | | | |
| Esophageal (N = 26) | 3.8% | 0 | 0 | 0 | 0 | 0 | 4.0% | 7.7% |
| Overall (N = 1029) | 5.0% | 1.6% | 1.6% | 0 | 0.8% | 0.8% | 6.3% | 15.2% |

Conclusion: Mutation rates of at least 8 to 43% on the HR pathway are reported from 13 cancer types. This method can potentially identify responders to DNA-damaging agents including platinum.

### Example 7: Calculating Microsatellite Instability from Next Generation Sequencing Results

Microsatellite instability status by Next Generation Sequencing (MSI-NGS) is measured by the direct analysis of known microsatellite regions sequenced in the NGS panel of the invention, presented in Tables 6-10 and accompanying text. This approach allows us to combine NGS analysis to assess multiple characteristics, including without limitation mutations, indels, copy number, fusions, and MSI.

To establish clinical thresholds, MSI-NGS results were compared with results from over 2,000 matching clinical cases analyzed with traditional, PCR-based methods. Genomic variants in the microsatellite loci are detected using the same depth and frequency criteria as used for mutation detection. Only insertions and deletions resulting in a change in the number of tandem repeats are considered in this assay. Some microsatellite regions with known polymorphisms or technical sequencing issues are excluded from the analysis. The total number of microsatellite alterations in each sample are counted and grouped into two categories: MSI-High and MSI-Stable. MSI-Low results are reported in the Stable category.

Each sample was identified as follows:

MSI-H—Defined as ≥65 incidents of difference from the expected nucleotide at any given region in the approximately 720 surveyed regions of the genome concerning microsatellite instability.

MS Stable (MSS)—Defined as <65 incidents of difference from the expected nucleotide at any given region in the approximately 720 surveyed regions of the genome concerning microsatellite instability.

Any ambiguous result that is less than the 99% confidence interval cutoff is considered as “MS Stable (MSS).” Any ambiguous result where there was an insufficient number of reads to be analyzed is considered as “Quantity not Sufficient (QNS).”

Comparison of MSI calculated by the gold standard fragment analysis (FA) compared to the MSI-NGS approach of the invention is shown in Table 15. Statistical analysis of the testing for all lineages is shown in Table 14. In the table, the statistics are calculated using fragment analysis as the gold standard.

TABLE 14
Statistical analysis of MSI-NGS

| | Samples | % |
|---|---|---|
| Lineage | Tested | Concordant |
| Bladder Cancer | 3 | 100.0% |
| Breast Carcinoma | 16 | 93.8% |
| Cholangiocarcinoma | 17 | 100.0% |
| Colorectal Adenocarcinoma | 1196 | 99.8% |
| Esophageal and Esophagogastric | 7 | 100.0% |
| Junction Carcinoma | | |
| Extrahepatic Bile Duct | 2 | 100.0% |
| Adenocarcinoma | | |
| Female Genital Tract Malignancy | 809 | 97.7% |
| Gastric Adenocarcinoma | 10 | 100.0% |
| Gastrointestinal Stromal Tumors | 2 | 100.0% |
| (GIST) | | |
| Glioblastoma | 9 | 100.0% |
| Liver Hepatocellular Carcinoma | 8 | 100.0% |
| Lung Non-small cell lung cancer | 5 | 100.0% |
| (NSCLC) | | |
| Lymphoma | 2 | 100.0% |
| Malignant Solitary Fibrous | 1 | 100.0% |
| Tumor of the Pleura (MSFT) | | |
| Melanoma | 4 | 100.0% |
| Neuroendocrine tumors | 10 | 100.0% |
| None Of Others Apply | 21 | 100.0% |
| Ovarian Surface Epithelial | 15 | 100.0% |
| Carcinomas | | |
| Pancreatic Adenocarcinoma | 44 | 97.7% |
| Prostatic Adenocarcinoma | 1 | 100.0% |
| Small Intestinal Malignancies | 7 | 100.0% |
| Soft Tissue Tumors | 1 | 100.0% |
| Thyroid Carcinoma | 1 | 100.0% |
| Uveal Melanoma | 1 | 100.0% |

TABLE 15
MSI FA vs. NGS Accuracy Summary

| Sample Set | Sensitivity | Specificity | PPV | NPV |
|---|---|---|---|---|
| All tumors | 94.9% | 99.4% | 94.5% | 99.1% |
| Colorectal | 100.0% | 99.8% | 97.4% | 99.6% |
| Cancer (CRC) | | | | |
| only | | | | |
| All lineages other | 92.2% | 98.8% | 92.9% | 98.5% |
| than CRC | | | | |

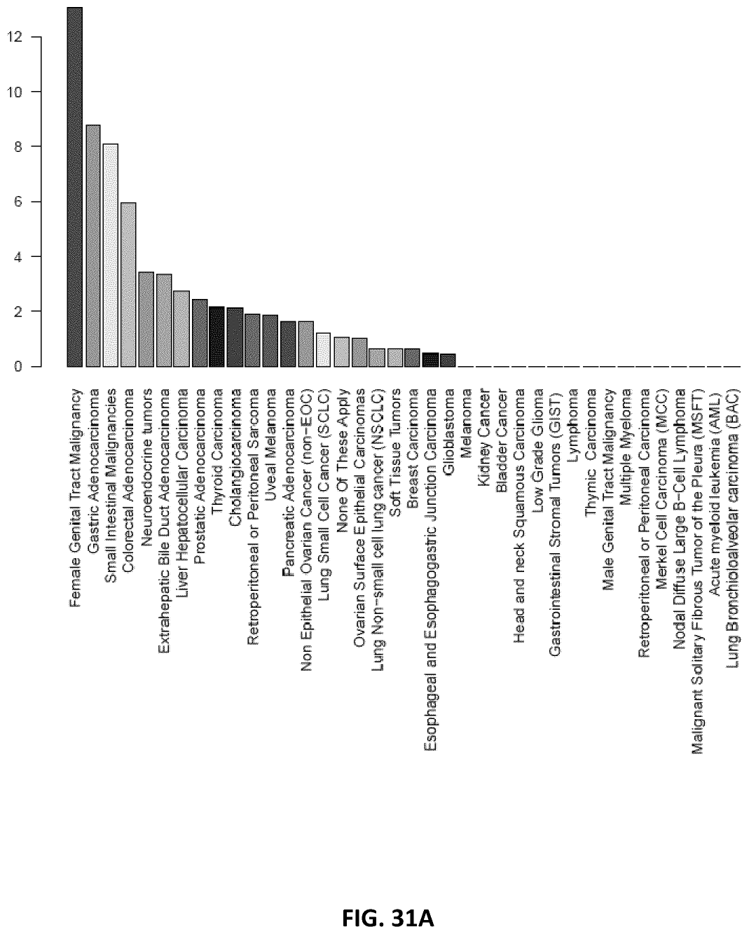

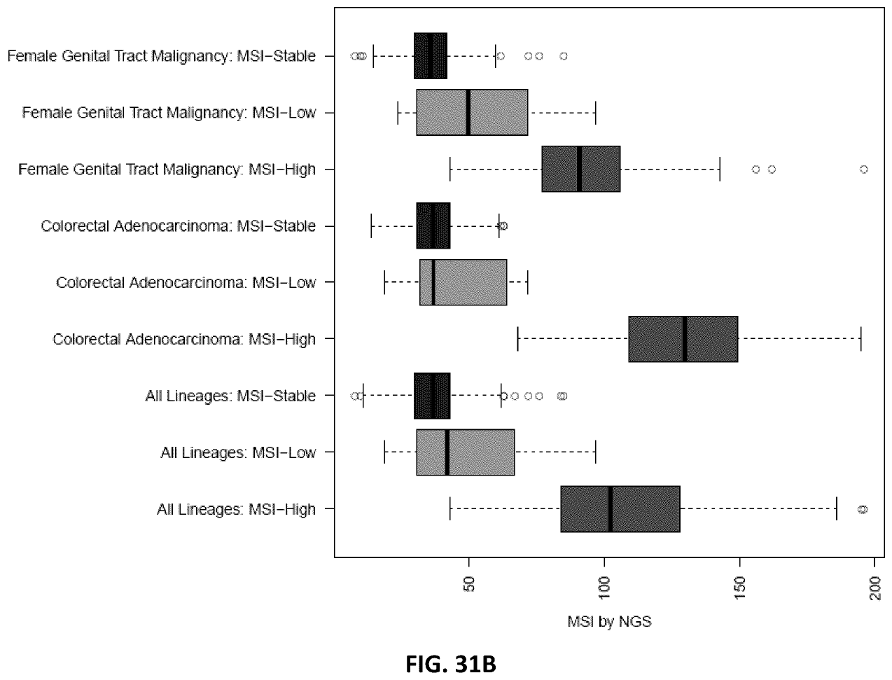

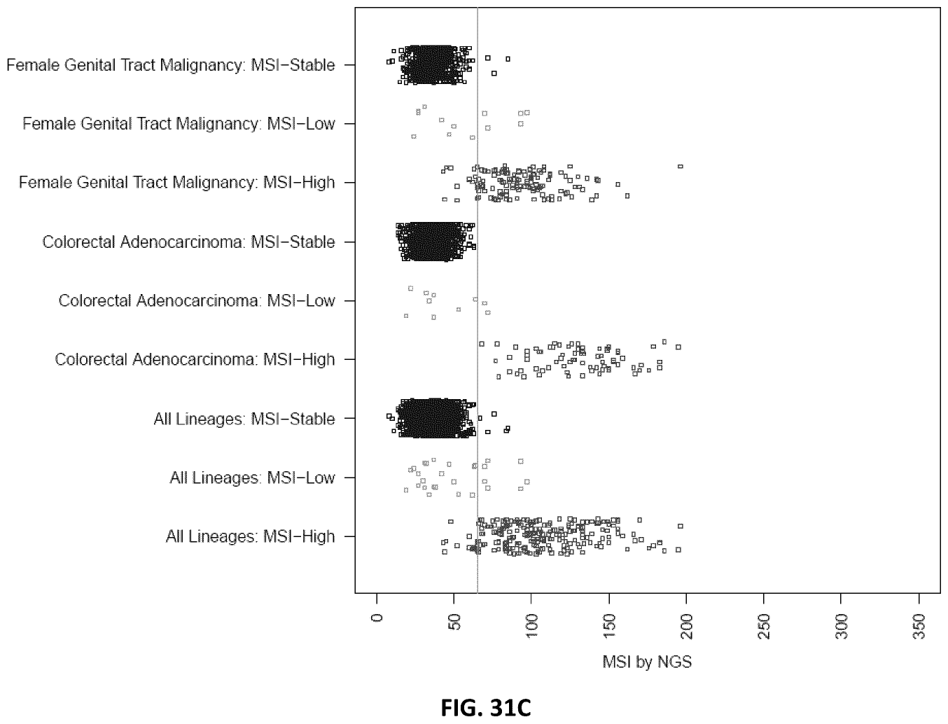

Frequency of MSI-H determined by NGS across multiple tumor lineages is shown in FIG. 31A. A box plot showing frequency within specified tumor types (female genital tract, colorectal, or all) is shown in FIG. 31B. A scatter plot showing the same is shown in FIG. 31C.

We also determined tumor mutation load (TML; also referred to as tumor mutation burden or TMB) using the same Next Generation Sequencing (NGS) analysis. TML was performed based on NGS analysis from genomic DNA isolated from a formalin-fixed paraffin-embedded tumor sample using the Illumina NextSeq platform.

Total mutational load was calculated using only missense mutations that have not been previously reported as germline alterations. Like MSI-H, high mutational load is a potential indicator of immunotherapy response. We defined threshold levels for Total Mutational Load and establish cutoff points:
High: greater than or equal to 17 mutations/Megabase (≥17 mutations/Mb). Approximately 7% of our molecular profiling cases reported a High result.
Intermediate: greater than or equal to 7 but fewer than 17 mutations/Megabase (≥7 and <17 mutations/Mb). Approximately 34% of our molecular profiling cases reported an Intermediate result.
Low: less than or equal to 6 mutations/Megabase (≤6 mutations/Mb). Approximately 59% of our molecular profiling cases reported a Low result.

### Example 8: Microsatellite Instability Status Determined by Next-Generation Sequencing and Compared with PD-L1 and Tumor Mutational Burden in 11,348 Patients

This Example is related to the Example above and presents additional assessment of microsatellite instability, PD-L1 and tumor mutational load in 11,251 patients across 31 tumor types.

### SUMMARY

Microsatellite instability (MSI) testing identifies patients who may benefit from immune checkpoint inhibitors. In this Example, we developed an MSI assay that uses data from a next-generation sequencing (NGS) panel to determine MSI status. The assay is applicable across cancer types and does not require matched samples from normal tissue. This Example describes the MSI-NGS method and explores the relationship of MSI with tumor mutational burden (TMB, also referred to as tumor mutational load or TML) and PD-L1. MSI examined by PCR fragment analysis and NGS was compared for 2,189 matched cases. Mismatch repair status by immunohistochemistry was compared to MSI-NGS for 1,986 matched cases. TMB was examined by NGS and PD-L1 was determined by immunohistochemistry (IHC). Among 2,189 matched cases that spanned 26 cancer types, MSI-NGS, as compared to MSI by PCR fragment analysis, had sensitivity of 95.8% (95% confidence interval [CI]92.24, 98.08), specificity of 99.4% (95% CI 98.94, 99.69), positive predictive value of 94.5% (95% CI 90.62, 97.14), and negative predictive value of 99.2% (95% CI, 98.75, 99.57). High MSI (MSI-H) status was identified in 23 of 26 cancer types. Among 11,348 cases examined (including the 2,189 matched cases), the overall rates of MSI-H, TMB-high and PD-L1 positivity were 3.0%, 7.7%, and 25.4%, respectively. Thirty percent of MSI-H cases were TMB-low and only 26% of MSI-H cases were PD-L1 positive. The overlap between TMB, MSI, and PD-L1 differed among cancer types. Only 0.6% of the cases were positive for all three markers. This Example shows that MSI-H status can be determined by NGS across cancer types, and that MSI-H offers distinct data for treatment decisions regarding immune checkpoint inhibitors, in addition to the data available from TMB and PD-L1. Thus, the techniques are complementary.

### INTRODUCTION

Microsatellite instability (MSI) involves the gain or loss of nucleotides from microsatellite tracts, which are DNA elements composed of repeating motifs that occur as alleles of variable lengths. [1] MSI can result from inherited mutations or originate somatically. Lynch syndrome results from inherited mutations of known mismatch repair (MMR) genes. Tumors are classified as MMR-deficient (dMMR) if they have somatic or germline mutations. MSI can also occur due to epigenetic changes or altered microRNA pathways affecting MMR proteins, or without a loss of a known underlying protein. [2] MSI is most commonly found in colon and endometrial cancers (the most common Lynch syndrome cancer types). However, recent analyses have found MSI in at least 24 cancer types, demonstrating that MSI is a generalized cancer phenotype. [3-6]

MSI has been associated with improved prognosis, but until the recent advent of immune checkpoint inhibitors, the predictive use of MSI has been limited. A proof-of-concept study including 87 patients with 12 different cancer types demoednstrat the predictive value of MSI status to predict response of solid tumors to the anti-PD-1 agent pembrolizumab. [5,7] This ability of MSI to predict pembrolizumab response has led to the first tumor-agnostic drug approval by the FDA in May 2017. Additional evidence showed an improved response for MSI-high (MSI-H) patients to the anti-PD-1 agents nivolumab and MEDI0680, the anti-PD-L1 agent durvalumab, and the anti-CTLA-4 agent ipilimumab. [7-10]

These results elevate MSI status as a third, possibly independent, predictive biomarker for immune checkpoint inhibitors, along with PD-L1 and tumor mutational burden (TMB). [11-17] Given that patient responses to these drugs can be highly durable, [5,7,18] it is critical to identify as many potential responders as possible. Therefore, a method to efficiently determine MSI status for every cancer patient is needed.

Currently, MSI is most commonly detected through polymerase chain reaction (PCR) by fragment analysis (FA) of five conserved satellite regions, which is considered the gold standard method for MSI detection. [1, 19] However, FA is not ideal in the clinic as it requires samples of both tumor and normal tissue. As a result, FA is not always feasible for cases with limited amounts of tissue, including the analysis of cancer metastases, which are commonly submitted as biopsies and may contain few normal cells. Additionally, determining MSI by FA and MMR analysis from immunohistochemistry (IHC) are performed as stand-alone tests and would be inefficient to perform on every cancer patient because the incidence of MSI is only about 5% across cancer types. [5]

As broad tumor profiling becomes a common part of care for cancer patients, it is preferable to determine MSI status from sequencing panel results. Next-generation sequencing (NGS) was recently found to be feasible to determine MSI status, but the published techniques also require the use of paired tumor and normal tissue. [3,6] We have access to a large database of samples with both broad NGS results and matching MSI status by FA and dMMR status by IHC. These data were obtained using the molecular profiling systems and methods of the invention. See, e.g., Tables 5-11 and related discussion. We used this database to develop and validate an NGS-based MSI assay without the need for matched samples from normal tissue. In this Example, we describe our process for developing such a method and explore the relationship of MSI with other immunotherapy markers, specifically TMB and PD-L1.

### Methods

### Patient Cohort

For development of the NGS assay, 2,189 cases were retrospectively selected based on having data available for both the 592-gene sequencing panel (see Tables 7-10) and MSI testing by PCR FA (assay details below). For the TMB, PD-L1, and MSI-NGS comparison, 11,348 patients were retrospectively selected based on available data from commercial comprehensive sequencing profiles performed on their tumors by our commercial laboratory (Caris Life Sciences, Phoenix, Arizona) that included PD-L1 by immunohistochemistry (IHC) and the 592-NGS gene sequencing panel. This research used a collection of existing data that were de-identified prior to analysis. As this research was compliant with 45 CFR 46.101(b), the project was deemed exempt from IRB oversight and consent requirements were waived.

### Fragment Analysis by PCR

MSI-FA was tested by the fluorescent multiplex PCR-based method (MSI Analysis; Promega, Life Sciences, Madison, WI, USA).

### Next-Generation Sequencing

NGS was performed on genomic DNA isolated from formalin-fixed paraffin-embedded (FFPE) tumor samples using the NextSeq platform (Illumina, Inc., San Diego, CA). A custom-designed SureSelect XT assay (Agilent Technologies, Santa Clara, CA) was used to enrich the 592 whole-gene targets that a 592-gene NGS panel. All variants were detected with >99% confidence based on allele frequency and baited-capture pull-down coverage with an average sequencing depth of over 50OX and with analytic sensitivity of 5% variant frequency.

### Microsatellite Instability by NGS

Microsatellite loci in the target regions of a 592-gene NGS panel were first identified using the MISA algorithm (pgrc.ipk-gatersleben.de/misa/), which revealed 8,921 microsatellite locations. Subsequent analyses excluded sex chromosome loci, microsatellite loci in regions that typically have lower coverage depth relative to other genomic regions, and microsatellites with repeat unit lengths greater than 5 nucleotides. These exclusions resulted in 7,317 target microsatellite loci. See Table 16 for positions of the loci. In the table, column “Chr” is the chromosome, “Start” and “End” are the position of the loci, and “MS” is information about the microsatellite.

TABLE 16
Microsatellite Loci analyzed by NGS

| Chr | Start | End | MS |
|---|---|---|---|
| 1 | 2488123 | 2488127 | p1.(G)5 |
| 1 | 2488147 | 2488151 | p1.(C)5 |
| 1 | 2488178 | 2488182 | p1.(C)5 |
| 1 | 2489156 | 2489160 | p1.(C)5 |
| 1 | 2489190 | 2489194 | p1.(C)5 |
| 1 | 2492097 | 2492102 | p1.(C)6 |
| 1 | 3102806 | 3102810 | p1.(C)5 |
| 1 | 3102833 | 3102837 | p1.(C)5 |
| 1 | 3102939 | 3102943 | p1.(G)5 |
| 1 | 3301766 | 3301770 | p1.(G)5 |
| 1 | 3313126 | 3313130 | p1.(C)5 |
| 1 | 3319446 | 3319450 | p1.(G)5 |
| 1 | 3328152 | 3328156 | p1.(C)5 |
| 1 | 3328185 | 3328189 | p1.(C)5 |
| 1 | 3328293 | 3328297 | p1.(C)5 |
| 1 | 3328330 | 3328334 | p1.(C)5 |
| 1 | 3328372 | 3328376 | p1.(C)5 |
| 1 | 3328709 | 3328713 | p1.(G)5 |
| 1 | 3328724 | 3328733 | p1.(C)5(G)5 |
| 1 | 3328960 | 3328964 | p1.(C)5 |
| 1 | 3329142 | 3329146 | p1.(C)5 |
| 1 | 3329312 | 3329316 | p1.(C)5 |
| 1 | 3331205 | 3331209 | p1.(C)5 |
| 1 | 3334475 | 3334479 | p1.(C)5 |
| 1 | 3334506 | 3334510 | p1.(C)5 |
| 1 | 3350287 | 3350291 | p1.(C)5 |
| 1 | 3350390 | 3350394 | p1.(G)5 |
| 1 | 6253104 | 6253108 | p1.(A)5 |
| 1 | 6257785 | 6257792 | p1.(T)8 |
| 1 | 6257794 | 6257799 | p1.(C)6 |
| 1 | 6257812 | 6257816 | p1.(T)5 |
| 1 | 7309621 | 7309625 | p1.(A)5 |
| 1 | 7723490 | 7723495 | p1.(G)6 |
| 1 | 7723845 | 7723849 | p1.(C)5 |
| 1 | 7724102 | 7724106 | p1.(G)5 |
| 1 | 7724422 | 7724426 | p1.(C)5 |
| 1 | 7724439 | 7724443 | p1.(C)5 |
| 1 | 7724522 | 7724526 | p1.(G)5 |
| 1 | 7724959 | 7724964 | p1.(G)6 |
| 1 | 7724998 | 7725002 | p1.(G)5 |
| 1 | 7725042 | 7725046 | p1.(C)5 |
| 1 | 7725082 | 7725086 | p1.(C)5 |
| 1 | 7796391 | 7796396 | p1.(T)6 |
| 1 | 7798090 | 7798094 | p1.(A)5 |
| 1 | 7798545 | 7798549 | p1.(A)5 |
| 1 | 7811271 | 7811275 | p1.(A)5 |
| 1 | 7811329 | 7811336 | p1.(A)8 |
| 1 | 7815698 | 7815702 | p1.(C)5 |
| 1 | 7826532 | 7826536 | p1.(A)5 |
| 1 | 7826612 | 7826616 | p1.(T)5 |
| 1 | 11166625 | 11166629 | p1.(A)5 |
| 1 | 11167510 | 11167515 | p1.(A)6 |
| 1 | 11182071 | 11182082 | p3.(TCT)4 |
| 1 | 11184561 | 11184565 | p1.(T)5 |
| 1 | 11187710 | 11187714 | p1.(G)5 |
| 1 | 11188191 | 11188195 | p1.(A)5 |
| 1 | 11188513 | 11188517 | p1.(G)5 |
| 1 | 11188944 | 11188948 | p1.(C)5 |
| 1 | 11189012 | 11189016 | p1.(A)5 |
| 1 | 11199383 | 11199387 | p1.(T)5 |
| 1 | 11199496 | 11199500 | p1.(A)5 |
| 1 | 11206726 | 11206730 | p1.(T)5 |
| 1 | 11206853 | 11206862 | p2.(AC)5 |
| 1 | 11217213 | 11217217 | p1.(C)5 |
| 1 | 11259454 | 11259458 | p1.(G)5 |
| 1 | 11272384 | 11272388 | p1.(A)5 |
| 1 | 11273547 | 11273551 | p1.(C)5 |
| 1 | 11276287 | 11276291 | p1.(A)5 |
| 1 | 11290970 | 11290974 | p1.(C)5 |
| 1 | 11292544 | 11292548 | p1.(A)5 |
| 1 | 11294235 | 11294240 | p1.(G)6 |
| 1 | 11303189 | 11303193 | p1.(G)5 |
| 1 | 11307945 | 11307949 | p1.(G)5 |
| 1 | 11308047 | 11308051 | p1.(G)5 |
| 1 | 11316169 | 11316173 | p1.(A)5 |
| 1 | 11316256 | 11316261 | p1.(A)6 |
| 1 | 11318597 | 11318601 | p1.(A)5 |
| 1 | 16174523 | 16174527 | p1.(G)5 |
| 1 | 16199301 | 16199306 | p1.(T)6 |
| 1 | 16199579 | 16199583 | p1.(C)5 |
| 1 | 16202753 | 16202757 | p1.(G)5 |
| 1 | 16202886 | 16202890 | p1.(C)5 |
| 1 | 16203144 | 16203155 | p3.(CAG)4 |
| 1 | 16235807 | 16235811 | p1.(T)5 |
| 1 | 16235914 | 16235918 | p1.(A)5 |
| 1 | 16237722 | 16237726 | p1.(A)5 |
| 1 | 16242716 | 16242721 | p1.(A)6 |
| 1 | 16245412 | 16245417 | p1.(T)6 |
| 1 | 16247345 | 16247356 | p4.(TTTG)3 |
| 1 | 16248729 | 16248739 | p1.(T)11 |
| 1 | 16248776 | 16248780 | p1.(T)5 |
| 1 | 16254575 | 16254580 | p1.(T)6 |
| 1 | 16255093 | 16255098 | p1.(A)6 |
| 1 | 16255142 | 16255153 | p2.(GA)6 |
| 1 | 16255170 | 16255174 | p1.(A)5 |
| 1 | 16255340 | 16255344 | p1.(A)5 |
| 1 | 16255728 | 16255732 | p1.(A)5 |
| 1 | 16255783 | 16255788 | p1.(A)6 |
| 1 | 16255883 | 16255889 | p1.(A)7 |
| 1 | 16256044 | 16256049 | p1.(A)6 |
| 1 | 16256126 | 16256130 | p1.(A)5 |
| 1 | 16256205 | 16256210 | p1.(A)6 |
| 1 | 16256321 | 16256326 | p1.(A)6 |
| 1 | 16256375 | 16256379 | p1.(C)5 |
| 1 | 16256411 | 16256415 | p1.(A)5 |
| 1 | 16256610 | 16256614 | p1.(A)5 |
| 1 | 16256833 | 16256837 | p1.(A)5 |
| 1 | 16256950 | 16256955 | p1.(T)6 |
| 1 | 16257221 | 16257225 | p1.(A)5 |
| 1 | 16257322 | 16257327 | p1.(T)6 |
| 1 | 16257525 | 16257529 | p1.(A)5 |
| 1 | 16257531 | 16257535 | p1.(A)5 |
| 1 | 16257842 | 16257846 | p1.(C)5 |
| 1 | 16258130 | 16258134 | p1.(C)5 |
| 1 | 16258181 | 16258185 | p1.(A)5 |
| 1 | 16258284 | 16258288 | p1.(A)5 |
| 1 | 16258376 | 16258380 | p1.(A)5 |
| 1 | 16258727 | 16258731 | p1.(A)5 |
| 1 | 16258735 | 16258740 | p1.(A)6 |
| 1 | 16258789 | 16258793 | p1.(C)5 |
| 1 | 16258889 | 16258899 | p1.(A)5(C)6 |
| 1 | 16258928 | 16258933 | p1.(A)6 |
| 1 | 16258944 | 16258950 | p1.(A)7 |
| 1 | 16259015 | 16259019 | p1.(A)5 |
| 1 | 16259043 | 16259048 | p1.(G)6 |
| 1 | 16259480 | 16259485 | p1.(C)6 |
| 1 | 16260017 | 16260021 | p1.(C)5 |
| 1 | 16260029 | 16260033 | p1.(C)5 |
| 1 | 16260194 | 16260199 | p1.(G)6 |
| 1 | 16260214 | 16260219 | p1.(C)6 |
| 1 | 16260290 | 16260294 | p1.(C)5 |
| 1 | 16260452 | 16260456 | p1.(C)5 |
| 1 | 16260470 | 16260474 | p1.(A)5 |
| 1 | 16260486 | 16260491 | p1.(A)6 |
| 1 | 16261246 | 16261251 | p1.(C)6 |
| 1 | 16261550 | 16261554 | p1.(C)5 |
| 1 | 16262460 | 16262464 | p1.(C)5 |
| 1 | 16262466 | 16262470 | p1.(C)5 |
| 1 | 16262478 | 16262482 | p1.(C)5 |
| 1 | 16262497 | 16262501 | p1.(C)5 |
| 1 | 16262553 | 16262557 | p1.(C)5 |
| 1 | 16262680 | 16262685 | p1.(C)6 |
| 1 | 16264073 | 16264077 | p1.(C)5 |
| 1 | 16264430 | 16264434 | p1.(C)5 |
| 1 | 16265971 | 16265975 | p1.(A)5 |
| 1 | 17355139 | 17355143 | p1.(T)5 |
| 1 | 17371390 | 17371395 | p1.(T)6 |
| 1 | 18958059 | 18958063 | p1.(T)5 |
| 1 | 18958157 | 18958161 | p1.(C)5 |
| 1 | 18961629 | 18961633 | p1.(A)5 |
| 1 | 19027261 | 19027266 | p1.(C)6 |
| 1 | 19027292 | 19027303 | p3.(CCA)4 |
| 1 | 19029590 | 19029594 | p1.(G)5 |
| 1 | 19062159 | 19062163 | p1.(C)5 |
| 1 | 19062397 | 19062401 | p1.(C)5 |
| 1 | 19062421 | 19062425 | p1.(C)5 |
| 1 | 19062496 | 19062500 | p1.(A)5 |
| 1 | 27023290 | 27023294 | p1.(G)5 |
| 1 | 27023327 | 27023331 | p1.(C)5 |
| 1 | 27023377 | 27023388 | p3.(CGC)4 |
| 1 | 27023451 | 27023462 | p3.(GCG)4 |
| 1 | 27023462 | 27023466 | p1.(G)5 |
| 1 | 27023560 | 27023565 | p1.(C)6 |
| 1 | 27023716 | 27023721 | p1.(G)6 |
| 1 | 27023744 | 27023748 | p1.(G)5 |
| 1 | 27023769 | 27023773 | p1.(C)5 |
| 1 | 27023831 | 27023835 | p1.(G)5 |
| 1 | 27023861 | 27023865 | p1.(G)5 |
| 1 | 27023904 | 27023909 | p1.(G)6 |
| 1 | 27024002 | 27024007 | p1.(G)6 |
| 1 | 27057727 | 27057738 | p3.(CAG)4 |
| 1 | 27057924 | 27057928 | p1.(C)5 |
| 1 | 27057937 | 27057942 | p1.(C)6 |
| 1 | 27059207 | 27059211 | p1.(C)5 |
| 1 | 27088659 | 27088663 | p1.(C)5 |
| 1 | 27088682 | 27088687 | p1.(C)6 |
| 1 | 27088788 | 27088793 | p1.(G)6 |
| 1 | 27089697 | 27089701 | p1.(C)5 |
| 1 | 27089706 | 27089710 | p1.(G)5 |
| 1 | 27092740 | 27092744 | p1.(G)5 |
| 1 | 27092815 | 27092819 | p1.(C)5 |
| 1 | 27093065 | 27093069 | p1.(T)5 |
| 1 | 27097622 | 27097627 | p1.(A)6 |
| 1 | 27097688 | 27097692 | p1.(A)5 |
| 1 | 27097751 | 27097755 | p1.(C)5 |
| 1 | 27099103 | 27099108 | p1.(C)6 |
| 1 | 27100176 | 27100181 | p1.(C)6 |
| 1 | 27100182 | 27100205 | p3.(GCA)8 |
| 1 | 27100919 | 27100930 | p3.(CAG)4 |
| 1 | 27100934 | 27100938 | p1.(C)5 |
| 1 | 27101068 | 27101072 | p1.(C)5 |
| 1 | 27101117 | 27101121 | p1.(C)5 |
| 1 | 27101268 | 27101273 | p1.(C)6 |
| 1 | 27101375 | 27101379 | p1.(C)5 |
| 1 | 27101402 | 27101407 | p1.(C)6 |
| 1 | 27101417 | 27101421 | p1.(C)5 |
| 1 | 27101438 | 27101442 | p1.(C)5 |
| 1 | 27101570 | 27101574 | p1.(C)5 |
| 1 | 27101612 | 27101617 | p1.(C)6 |
| 1 | 27105507 | 27105511 | p1.(T)5 |
| 1 | 27105676 | 27105690 | p3.(GAA)5 |
| 1 | 27105931 | 27105937 | p1.(G)7 |
| 1 | 27106078 | 27106082 | p1.(C)5 |
| 1 | 27106100 | 27106104 | p1.(A)5 |
| 1 | 27106804 | 27106809 | p1.(C)6 |
| 1 | 27106917 | 27106921 | p1.(G)5 |
| 1 | 27107227 | 27107231 | p1.(T)5 |
| 1 | 32740658 | 32740662 | p1.(G)5 |
| 1 | 32741500 | 32741511 | p4.(CATT)3 |
| 1 | 32742059 | 32742063 | p1.(G)5 |
| 1 | 35650015 | 35650021 | p1.(A)7 |
| 1 | 35650069 | 35650074 | p1.(T)6 |
| 1 | 35652860 | 35652866 | p1.(A)7 |
| 1 | 35654780 | 35654784 | p1.(T)5 |
| 1 | 35654867 | 35654873 | p1.(T)7 |
| 1 | 35654893 | 35654897 | p1.(T)5 |
| 1 | 35656128 | 35656132 | p1.(T)5 |
| 1 | 35656203 | 35656207 | p1.(A)5 |
| 1 | 35656978 | 35656982 | p1.(A)5 |
| 1 | 35657137 | 35657141 | p1.(A)5 |
| 1 | 35657872 | 35657876 | p1.(G)5 |
| 1 | 35657920 | 35657924 | p1.(C)5 |
| 1 | 35658230 | 35658234 | p1.(C)5 |
| 1 | 36748170 | 36748174 | p1.(A)5 |
| 1 | 36748270 | 36748279 | p2.(TC)5 |
| 1 | 36748306 | 36748310 | p1.(G)5 |
| 1 | 36752369 | 36752373 | p1.(A)5 |
| 1 | 36752590 | 36752594 | p1.(C)5 |
| 1 | 36752768 | 36752772 | p1.(A)5 |
| 1 | 36754648 | 36754652 | p1.(T)5 |
| 1 | 36754729 | 36754734 | p1.(A)6 |
| 1 | 36754778 | 36754782 | p1.(A)5 |
| 1 | 36755001 | 36755005 | p1.(A)5 |
| 1 | 36755075 | 36755079 | p1.(A)5 |
| 1 | 36755118 | 36755122 | p1.(A)5 |
| 1 | 36755229 | 36755233 | p1.(C)5 |
| 1 | 36756967 | 36756971 | p1.(T)5 |
| 1 | 36757033 | 36757037 | p1.(A)5 |
| 1 | 36758282 | 36758286 | p1.(A)5 |
| 1 | 36759466 | 36759470 | p1.(C)5 |
| 1 | 36767233 | 36767238 | p1.(A)6 |
| 1 | 36931673 | 36931677 | p1.(A)5 |
| 1 | 36931687 | 36931691 | p1.(A)5 |
| 1 | 36932008 | 36932012 | p1.(G)5 |
| 1 | 36932123 | 36932127 | p1.(G)5 |
| 1 | 36932275 | 36932279 | p1.(C)5 |
| 1 | 36932921 | 36932925 | p1.(G)5 |
| 1 | 36933724 | 36933728 | p1.(G)5 |
| 1 | 36933744 | 36933748 | p1.(G)5 |
| 1 | 36935323 | 36935329 | p1.(G)7 |
| 1 | 36935371 | 36935376 | p1.(G)6 |
| 1 | 36937024 | 36937028 | p1.(C)5 |
| 1 | 36937736 | 36937740 | p1.(G)5 |
| 1 | 36937983 | 36937987 | p1.(G)5 |
| 1 | 36938271 | 36938276 | p1.(G)6 |
| 1 | 36941121 | 36941125 | p1.(C)5 |
| 1 | 36941221 | 36941225 | p1.(C)5 |
| 1 | 36941237 | 36941241 | p1.(G)5 |
| 1 | 40363033 | 40363037 | p1.(T)5 |
| 1 | 40363133 | 40363137 | p1.(C)5 |
| 1 | 40363185 | 40363189 | p1.(G)5 |
| 1 | 40363366 | 40363370 | p1.(G)5 |
| 1 | 40363553 | 40363557 | p1.(G)5 |
| 1 | 40366672 | 40366676 | p1.(G)5 |
| 1 | 40366747 | 40366751 | p1.(C)5 |
| 1 | 40366925 | 40366929 | p1.(G)5 |
| 1 | 40366939 | 40366943 | p1.(G)5 |
| 1 | 40366987 | 40366992 | p1.(G)6 |
| 1 | 40367129 | 40367134 | p1.(G)6 |
| 1 | 43804231 | 43804235 | p1.(C)5 |
| 1 | 43804953 | 43804958 | p1.(C)6 |
| 1 | 43814993 | 43815004 | p3.(CTG)4 |
| 1 | 43815037 | 43815041 | p1.(C)5 |
| 1 | 43817880 | 43817884 | p1.(C)5 |
| 1 | 45794921 | 45794925 | p1.(A)5 |
| 1 | 45794961 | 45794965 | p1.(G)5 |
| 1 | 45795066 | 45795070 | p1.(T)5 |
| 1 | 45795115 | 45795120 | p1.(A)6 |
| 1 | 45796855 | 45796859 | p1.(T)5 |
| 1 | 45797418 | 45797423 | p1.(G)6 |
| 1 | 45798340 | 45798344 | p1.(C)5 |
| 1 | 45798845 | 45798849 | p1.(G)5 |
| 1 | 45800191 | 45800195 | p1.(A)5 |
| 1 | 47685527 | 47685531 | p1.(G)5 |
| 1 | 47685558 | 47685562 | p1.(G)5 |
| 1 | 47685570 | 47685574 | p1.(C)5 |
| 1 | 47685576 | 47685580 | p1.(C)5 |
| 1 | 47685588 | 47685592 | p1.(C)5 |
| 1 | 47685598 | 47685602 | p1.(C)5 |
| 1 | 47685640 | 47685651 | p3.(CCT)4 |
| 1 | 47685732 | 47685736 | p1.(G)5 |
| 1 | 47691412 | 47691416 | p1.(G)5 |
| 1 | 47691475 | 47691481 | p1.(G)7 |
| 1 | 47691518 | 47691522 | p1.(G)5 |
| 1 | 47691569 | 47691580 | p4.(CAGA)3 |
| 1 | 47716916 | 47716920 | p1.(A)5 |
| 1 | 47717309 | 47717314 | p1.(T)6 |
| 1 | 47725985 | 47725989 | p1.(T)5 |
| 1 | 47726013 | 47726017 | p1.(T)5 |
| 1 | 47726091 | 47726097 | p1.(T)7 |
| 1 | 47726217 | 47726221 | p1.(A)5 |
| 1 | 47728643 | 47728647 | p1.(T)5 |
| 1 | 47728681 | 47728685 | p1.(C)5 |
| 1 | 47735440 | 47735444 | p1.(A)5 |
| 1 | 47746352 | 47746356 | p1.(G)5 |
| 1 | 47746401 | 47746406 | p1.(A)6 |
| 1 | 47746581 | 47746585 | p1.(G)5 |
| 1 | 47753233 | 47753237 | p1.(A)5 |
| 1 | 47753256 | 47753260 | p1.(A)5 |
| 1 | 47753310 | 47753314 | p1.(T)5 |
| 1 | 47755250 | 47755255 | p1.(A)6 |
| 1 | 47755264 | 47755270 | p1.(A)7 |
| 1 | 47767318 | 47767322 | p1.(A)5 |
| 1 | 47767932 | 47767936 | p1.(A)5 |
| 1 | 47767947 | 47767954 | p1.(T)8 |
| 1 | 47768022 | 47768026 | p1.(A)5 |
| 1 | 47770675 | 47770680 | p1.(A)6 |
| 1 | 51436054 | 51436058 | p1.(G)5 |
| 1 | 51436084 | 51436089 | p1.(G)6 |
| 1 | 51439843 | 51439847 | p1.(G)5 |
| 1 | 51439919 | 51439923 | p1.(G)5 |
| 1 | 51826933 | 51826937 | p1.(T)5 |
| 1 | 51829591 | 51829595 | p1.(G)5 |
| 1 | 51829633 | 51829638 | p1.(T)6 |
| 1 | 51829709 | 51829713 | p1.(A)5 |
| 1 | 51864840 | 51864846 | p1.(A)7 |
| 1 | 51869083 | 51869088 | p1.(T)6 |
| 1 | 51869130 | 51869134 | p1.(T)5 |
| 1 | 51875324 | 51875328 | p1.(T)5 |
| 1 | 51912754 | 51912758 | p1.(T)5 |
| 1 | 51913720 | 51913724 | p1.(T)5 |
| 1 | 51913797 | 51913801 | p1.(A)5 |
| 1 | 51926826 | 51926830 | p1.(A)5 |
| 1 | 59247730 | 59247734 | p1.(C)5 |
| 1 | 59247894 | 59247898 | p1.(T)5 |
| 1 | 59247929 | 59247933 | p1.(T)5 |
| 1 | 59248011 | 59248015 | p1.(G)5 |
| 1 | 59248124 | 59248138 | p3.(GCT)5 |
| 1 | 59248285 | 59248289 | p1.(C)5 |
| 1 | 59248461 | 59248465 | p1.(G)5 |
| 1 | 65303790 | 65303799 | p2.(GA)5 |
| 1 | 65304140 | 65304145 | p1.(T)6 |
| 1 | 65304281 | 65304286 | p1.(A)6 |
| 1 | 65305357 | 65305361 | p1.(T)5 |
| 1 | 65306997 | 65307004 | p1.(T)8 |
| 1 | 65310532 | 65310536 | p1.(T)5 |
| 1 | 65311293 | 65311297 | p1.(C)5 |
| 1 | 65325833 | 65325839 | p1.(G)7 |
| 1 | 65330547 | 65330551 | p1.(A)5 |
| 1 | 65330576 | 65330580 | p1.(T)5 |
| 1 | 65330611 | 65330616 | p1.(T)6 |
| 1 | 65330630 | 65330636 | p1.(T)7 |
| 1 | 65339111 | 65339118 | p1.(T)8 |
| 1 | 65339129 | 65339133 | p1.(T)5 |
| 1 | 78414403 | 78414414 | p1.(T)7(C)5 |
| 1 | 78414991 | 78414998 | p1.(A)8 |
| 1 | 78425957 | 78425961 | p1.(A)5 |
| 1 | 78426120 | 78426124 | p1.(G)5 |
| 1 | 78426185 | 78426192 | p1.(A)8 |
| 1 | 78428598 | 78428602 | p1.(T)5 |
| 1 | 78429978 | 78429984 | p1.(T)7 |
| 1 | 78430049 | 78430054 | p1.(A)6 |
| 1 | 78430320 | 78430324 | p1.(T)5 |
| 1 | 78430775 | 78430779 | p1.(C)5 |
| 1 | 78430879 | 78430883 | p1.(T)5 |
| 1 | 78444659 | 78444664 | p1.(G)6 |
| 1 | 78444746 | 78444757 | p4.(AAGA)3 |
| 1 | 85733287 | 85733291 | p1.(A)5 |
| 1 | 85733513 | 85733519 | p1.(A)7 |
| 1 | 85733574 | 85733578 | p1.(T)5 |
| 1 | 85736376 | 85736381 | p1.(T)6 |
| 1 | 85736457 | 85736461 | p1.(T)5 |
| 1 | 85736511 | 85736518 | p1.(T)8 |
| 1 | 93297607 | 93297611 | p1.(C)5 |
| 1 | 93298933 | 93298938 | p1.(T)6 |
| 1 | 93299088 | 93299094 | p1.(T)7 |
| 1 | 93299149 | 93299153 | p1.(A)5 |
| 1 | 93300356 | 93300360 | p1.(G)5 |
| 1 | 93301898 | 93301902 | p1.(T)5 |
| 1 | 93306186 | 93306190 | p1.(A)5 |
| 1 | 110882250 | 110882254 | p1.(G)5 |
| 1 | 110882373 | 110882377 | p1.(G)5 |
| 1 | 110882421 | 110882425 | p1.(A)5 |
| 1 | 110882436 | 110882440 | p1.(G)5 |
| 1 | 110882873 | 110882878 | p1.(C)6 |
| 1 | 110882973 | 110882977 | p1.(C)5 |
| 1 | 110883792 | 110883796 | p1.(C)5 |
| 1 | 110883798 | 110883802 | p1.(C)5 |
| 1 | 110884185 | 110884189 | p1.(A)5 |
| 1 | 110884244 | 110884248 | p1.(A)5 |
| 1 | 110884257 | 110884262 | p1.(A)6 |
| 1 | 110884341 | 110884346 | p1.(G)6 |
| 1 | 110884751 | 110884756 | p1.(G)6 |
| 1 | 110888908 | 110888921 | p1.(T)7(C)7 |
| 1 | 110888983 | 110888987 | p1.(T)5 |
| 1 | 114942160 | 114942165 | p1.(T)6 |
| 1 | 114942176 | 114942180 | p1.(T)5 |
| 1 | 114948088 | 114948092 | p1.(T)5 |
| 1 | 114949592 | 114949597 | p1.(T)6 |
| 1 | 114952868 | 114952872 | p1.(G)5 |
| 1 | 114967382 | 114967386 | p1.(A)5 |
| 1 | 114968116 | 114968130 | p3.(TGT)5 |
| 1 | 114968349 | 114968353 | p1.(A)5 |
| 1 | 115006901 | 115006905 | p1.(T)5 |
| 1 | 115053195 | 115053200 | p1.(C)6 |
| 1 | 115053423 | 115053427 | p1.(C)5 |
| 1 | 115053499 | 115053505 | p1.(C)7 |
| 1 | 115053552 | 115053569 | p3.(TCC)6 |
| 1 | 115053651 | 115053655 | p1.(C)5 |
| 1 | 115251216 | 115251221 | p1.(T)6 |
| 1 | 115251257 | 115251261 | p1.(A)5 |
| 1 | 115252352 | 115252356 | p1.(A)5 |
| 1 | 115256602 | 115256606 | p1.(G)5 |
| 1 | 116916142 | 116916146 | p1.(G)5 |
| 1 | 116926684 | 116926688 | p1.(A)5 |
| 1 | 116926693 | 116926697 | p1.(A)5 |
| 1 | 116926702 | 116926707 | p1.(A)6 |
| 1 | 116929970 | 116929974 | p1.(C)5 |
| 1 | 116930018 | 116930023 | p1.(G)6 |
| 1 | 116930849 | 116930853 | p1.(A)6 |
| 1 | 116931592 | 116931596 | p1.(C)5 |
| 1 | 116931650 | 116931654 | p1.(T)5 |
| 1 | 116932053 | 116932057 | p1.(T)5 |
| 1 | 116932151 | 116932155 | p1.(C)5 |
| 1 | 116932827 | 116932832 | p1.(T)6 |
| 1 | 116932871 | 116932875 | p1.(A)5 |
| 1 | 116936320 | 116936325 | p1.(G)6 |
| 1 | 116947067 | 116947072 | p1.(C)6 |
| 1 | 116947123 | 116947127 | p1.(C)5 |
| 1 | 118166214 | 118166218 | p1.(G)5 |
| 1 | 120458005 | 120458009 | p1.(C)5 |
| 1 | 120458185 | 120458189 | p1.(G)5 |
| 1 | 120458339 | 120458343 | p1.(G)5 |
| 1 | 120458384 | 120458388 | p1.(C)5 |
| 1 | 120458436 | 120458441 | p1.(G)6 |
| 1 | 120458741 | 120458745 | p1.(A)5 |
| 1 | 120460319 | 120460323 | p1.(T)5 |
| 1 | 120464983 | 120464987 | p1.(C)5 |
| 1 | 120466363 | 120466367 | p1.(C)5 |
| 1 | 120468081 | 120468085 | p1.(C)5 |
| 1 | 120468185 | 120468190 | p1.(G)6 |
| 1 | 120468279 | 120468283 | p1.(C)5 |
| 1 | 120468376 | 120468380 | p1.(C)5 |
| 1 | 120469123 | 120469127 | p1.(G)5 |
| 1 | 120480012 | 120480016 | p1.(G)5 |
| 1 | 120480571 | 120480576 | p1.(T)6 |
| 1 | 120480596 | 120480600 | p1.(T)5 |
| 1 | 120483185 | 120483189 | p1.(T)5 |
| 1 | 120483204 | 120483208 | p1.(G)5 |
| 1 | 120496255 | 120496259 | p1.(T)5 |
| 1 | 120508193 | 120508197 | p1.(A)5 |
| 1 | 120510781 | 120510785 | p1.(G)5 |
| 1 | 120510802 | 120510806 | p1.(C)5 |
| 1 | 120512304 | 120512308 | p1.(C)5 |
| 1 | 120512374 | 120512378 | p1.(G)5 |
| 1 | 120529594 | 120529598 | p1.(G)5 |
| 1 | 144873902 | 144873906 | p1.(G)5 |
| 1 | 144873958 | 144873962 | p1.(C)5 |
| 1 | 144877213 | 144877218 | p1.(T)6 |
| 1 | 144879086 | 144879091 | p1.(T)6 |
| 1 | 144879143 | 144879147 | p1.(T)5 |
| 1 | 144886269 | 144886273 | p1.(A)5 |
| 1 | 144906113 | 144906117 | p1.(T)5 |
| 1 | 144909858 | 144909863 | p1.(A)6 |
| 1 | 144909884 | 144909889 | p1.(T)6 |
| 1 | 144909929 | 144909933 | p1.(T)5 |
| 1 | 144911966 | 144911971 | p1.(A)6 |
| 1 | 144912160 | 144912164 | p1.(G)5 |
| 1 | 144917592 | 144917596 | p1.(T)5 |
| 1 | 144917619 | 144917623 | p1.(T)5 |
| 1 | 144917941 | 144917945 | p1.(A)5 |
| 1 | 144923716 | 144923721 | p1.(T)6 |
| 1 | 144994767 | 144994771 | p1.(C)5 |
| 1 | 144994948 | 144994953 | p1.(T)6 |
| 1 | 147084716 | 147084720 | p1.(C)5 |
| 1 | 147084745 | 147084749 | p1.(C)5 |
| 1 | 147084784 | 147084789 | p1.(G)6 |
| 1 | 147084813 | 147084817 | p1.(C)5 |
| 1 | 147084833 | 147084837 | p1.(G)5 |
| 1 | 147086304 | 147086308 | p1.(C)5 |
| 1 | 147086319 | 147086323 | p1.(C)5 |
| 1 | 147090673 | 147090677 | p1.(C)5 |
| 1 | 147090769 | 147090773 | p1.(C)5 |
| 1 | 147090856 | 147090860 | p1.(C)5 |
| 1 | 147090982 | 147090987 | p1.(C)6 |
| 1 | 147091079 | 147091083 | p1.(T)5 |
| 1 | 147091117 | 147091121 | p1.(G)5 |
| 1 | 147091159 | 147091164 | p1.(A)6 |
| 1 | 147091501 | 147091508 | p1.(C)8 |
| 1 | 147091546 | 147091550 | p1.(G)5 |
| 1 | 147091594 | 147091598 | p1.(C)5 |
| 1 | 147091752 | 147091756 | p1.(A)5 |
| 1 | 147091830 | 147091834 | p1.(C)5 |
| 1 | 147091890 | 147091895 | p1.(C)6 |
| 1 | 147092053 | 147092057 | p1.(C)5 |
| 1 | 147092277 | 147092281 | p1.(C)5 |
| 1 | 147092615 | 147092620 | p1.(C)6 |
| 1 | 147092659 | 147092670 | p3.(GCT)4 |
| 1 | 147092681 | 147092687 | p1.(C)7 |
| 1 | 147094076 | 147094081 | p1.(C)6 |
| 1 | 147094090 | 147094101 | p4.(CAGC)3 |
| 1 | 147095634 | 147095638 | p1.(T)5 |
| 1 | 147095890 | 147095894 | p1.(C)5 |
| 1 | 147095918 | 147095922 | p1.(C)5 |
| 1 | 147095957 | 147095962 | p1.(G)6 |
| 1 | 147096004 | 147096008 | p1.(C)5 |
| 1 | 147096074 | 147096078 | p1.(G)5 |
| 1 | 147096321 | 147096325 | p1.(C)5 |
| 1 | 147096567 | 147096571 | p1.(G)5 |
| 1 | 147096667 | 147096672 | p1.(C)6 |
| 1 | 150549801 | 150549815 | p3.(TGG)5 |
| 1 | 150550940 | 150550944 | p1.(T)5 |
| 1 | 150551310 | 150551314 | p1.(C)5 |
| 1 | 150551492 | 150551503 | p3.(TCC)4 |
| 1 | 150551728 | 150551732 | p1.(G)5 |
| 1 | 150551810 | 150551815 | p1.(G)6 |
| 1 | 150551858 | 150551862 | p1.(C)5 |
| 1 | 150551940 | 150551944 | p1.(C)5 |
| 1 | 150551952 | 150551958 | p1.(C)7 |
| 1 | 150552014 | 150552025 | p3.(CGC)4 |
| 1 | 150789283 | 150789288 | p1.(G)6 |
| 1 | 150790388 | 150790393 | p1.(T)6 |
| 1 | 150795825 | 150795829 | p1.(A)5 |
| 1 | 150807080 | 150807084 | p1.(T)5 |
| 1 | 150825241 | 150825245 | p1.(A)5 |
| 1 | 151039875 | 151039879 | p1.(A)5 |
| 1 | 154130203 | 154130207 | p1.(G)5 |
| 1 | 154142948 | 154142952 | p1.(G)5 |
| 1 | 154148611 | 154148615 | p1.(T)5 |
| 1 | 155159732 | 155159736 | p1.(G)5 |
| 1 | 155160736 | 155160740 | p1.(A)5 |
| 1 | 156737669 | 156737673 | p1.(G)5 |
| 1 | 156737723 | 156737727 | p1.(C)5 |
| 1 | 156737750 | 156737754 | p1.(C)5 |
| 1 | 156737768 | 156737772 | p1.(C)5 |
| 1 | 156737804 | 156737809 | p1.(C)6 |
| 1 | 156737833 | 156737838 | p1.(C)6 |
| 1 | 156737930 | 156737934 | p1.(C)5 |
| 1 | 156737954 | 156737958 | p1.(C)5 |
| 1 | 156752062 | 156752066 | p1.(T)5 |
| 1 | 156756445 | 156756449 | p1.(A)5 |
| 1 | 156756698 | 156756702 | p1.(C)5 |
| 1 | 156756709 | 156756713 | p1.(T)5 |
| 1 | 156756839 | 156756844 | p1.(C)6 |
| 1 | 156761536 | 156761543 | p1.(C)8 |
| 1 | 156770304 | 156770308 | p1.(C)5 |
| 1 | 156830849 | 156830853 | p1.(C)5 |
| 1 | 156834203 | 156834207 | p1.(G)5 |
| 1 | 156836777 | 156836781 | p1.(G)5 |
| 1 | 156837888 | 156837893 | p1.(C)6 |
| 1 | 156838343 | 156838347 | p1.(G)5 |
| 1 | 156841421 | 156841425 | p1.(G)5 |
| 1 | 156844688 | 156844692 | p1.(C)5 |
| 1 | 156845387 | 156845391 | p1.(C)5 |
| 1 | 156845863 | 156845867 | p1.(C)5 |
| 1 | 156845918 | 156845922 | p1.(G)5 |
| 1 | 156846308 | 156846312 | p1.(C)5 |
| 1 | 156848968 | 156848972 | p1.(C)5 |
| 1 | 156851434 | 156851438 | p1.(G)5 |
| 1 | 157548320 | 157548324 | p1.(T)5 |
| 1 | 157556026 | 157556030 | p1.(C)5 |
| 1 | 157556200 | 157556204 | p1.(C)5 |
| 1 | 162724572 | 162724576 | p1.(C)5 |
| 1 | 162731105 | 162731109 | p1.(C)5 |
| 1 | 162741857 | 162741861 | p1.(G)5 |
| 1 | 162743287 | 162743291 | p1.(A)5 |
| 1 | 162745596 | 162745600 | p1.(C)5 |
| 1 | 164529037 | 164529041 | p1.(G)5 |
| 1 | 164529161 | 164529165 | p1.(G)5 |
| 1 | 164532540 | 164532545 | p1.(A)6 |
| 1 | 164761720 | 164761724 | p1.(T)5 |
| 1 | 164761771 | 164761775 | p1.(C)5 |
| 1 | 164781392 | 164781397 | p1.(T)6 |
| 1 | 164818582 | 164818586 | p1.(C)5 |
| 1 | 164818591 | 164818595 | p1.(C)5 |
| 1 | 170633350 | 170633354 | p1.(G)5 |
| 1 | 170633450 | 170633454 | p1.(A)5 |
| 1 | 170688888 | 170688894 | p1.(A)7 |
| 1 | 170695421 | 170695425 | p1.(A)5 |
| 1 | 170695521 | 170695525 | p1.(G)5 |
| 1 | 170699410 | 170699414 | p1.(A)5 |
| 1 | 170705330 | 170705337 | p1.(A)8 |
| 1 | 170705364 | 170705368 | p1.(A)5 |
| 1 | 179077188 | 179077192 | p1.(T)5 |
| 1 | 179077445 | 179077449 | p1.(G)5 |
| 1 | 179077639 | 179077643 | p1.(G)5 |
| 1 | 179077738 | 179077742 | p1.(G)5 |
| 1 | 179077894 | 179077900 | p1.(T)7 |
| 1 | 179078126 | 179078130 | p1.(T)5 |
| 1 | 179078174 | 179078178 | p1.(C)5 |
| 1 | 179078192 | 179078196 | p1.(C)5 |
| 1 | 179078198 | 179078202 | p1.(C)5 |
| 1 | 179078242 | 179078247 | p1.(C)6 |
| 1 | 179078404 | 179078409 | p1.(G)6 |
| 1 | 179078450 | 179078455 | p1.(C)6 |
| 1 | 179089414 | 179089418 | p1.(A)5 |
| 1 | 179090803 | 179090807 | p1.(C)5 |
| 1 | 179090862 | 179090866 | p1.(G)5 |
| 1 | 179095689 | 179095693 | p1.(T)5 |
| 1 | 186283862 | 186283866 | p1.(A)5 |
| 1 | 186287740 | 186287744 | p1.(A)5 |
| 1 | 186291530 | 186291536 | p1.(A)7 |
| 1 | 186292867 | 186292871 | p1.(G)5 |
| 1 | 186292967 | 186292971 | p1.(A)5 |
| 1 | 186294885 | 186294889 | p1.(A)5 |
| 1 | 186294989 | 186294993 | p1.(A)5 |
| 1 | 186296797 | 186296801 | p1.(A)5 |
| 1 | 186300619 | 186300623 | p1.(T)5 |
| 1 | 186301462 | 186301466 | p1.(G)5 |
| 1 | 186302486 | 186302490 | p1.(A)5 |
| 1 | 186302530 | 186302534 | p1.(A)5 |
| 1 | 186305643 | 186305647 | p1.(T)5 |
| 1 | 186305676 | 186305681 | p1.(T)6 |
| 1 | 186305812 | 186305816 | p1.(T)5 |
| 1 | 186307246 | 186307250 | p1.(T)5 |
| 1 | 186307380 | 186307385 | p1.(A)6 |
| 1 | 186310509 | 186310513 | p1.(T)5 |
| 1 | 186312587 | 186312591 | p1.(T)5 |
| 1 | 186312610 | 186312614 | p1.(A)5 |
| 1 | 186313145 | 186313149 | p1.(T)5 |
| 1 | 186313608 | 186313612 | p1.(T)5 |
| 1 | 186315318 | 186315322 | p1.(T)5 |
| 1 | 186316427 | 186316431 | p1.(T)5 |
| 1 | 186319434 | 186319438 | p1.(T)5 |
| 1 | 186319444 | 186319449 | p1.(T)6 |
| 1 | 186320543 | 186320547 | p1.(T)5 |
| 1 | 186321144 | 186321148 | p1.(C)5 |
| 1 | 186322991 | 186322996 | p1.(A)6 |
| 1 | 186324639 | 186324644 | p1.(T)6 |
| 1 | 186324655 | 186324659 | p1.(T)5 |
| 1 | 186324661 | 186324666 | p1.(T)6 |
| 1 | 186324676 | 186324680 | p1.(A)5 |
| 1 | 186325408 | 186325412 | p1.(A)5 |
| 1 | 186325588 | 186325592 | p1.(A)5 |
| 1 | 186326758 | 186326762 | p1.(A)5 |
| 1 | 186327661 | 186327667 | p1.(A)7 |
| 1 | 186329976 | 186329980 | p1.(T)5 |
| 1 | 186330040 | 186330045 | p1.(A)6 |
| 1 | 186331005 | 186331011 | p1.(A)7 |
| 1 | 186331975 | 186331980 | p1.(T)6 |
| 1 | 186332020 | 186332024 | p1.(T)5 |
| 1 | 186332122 | 186332126 | p1.(T)5 |
| 1 | 186332552 | 186332556 | p1.(T)5 |
| 1 | 186344286 | 186344297 | p4.(CGCC)3 |
| 1 | 193091320 | 193091327 | p1.(G)8 |
| 1 | 193091458 | 193091462 | p1.(G)5 |
| 1 | 193099382 | 193099386 | p1.(T)5 |
| 1 | 193111006 | 193111011 | p1.(A)6 |
| 1 | 193111146 | 193111155 | p2.(AG)5 |
| 1 | 193116999 | 193117003 | p1.(T)5 |
| 1 | 193117013 | 193117017 | p1.(T)5 |
| 1 | 193119423 | 193119429 | p1.(T)7 |
| 1 | 193121497 | 193121504 | p1.(T)8 |
| 1 | 193202112 | 193202117 | p1.(T)6 |
| 1 | 193202211 | 193202215 | p1.(G)5 |
| 1 | 193205403 | 193205407 | p1.(T)5 |
| 1 | 198675866 | 198675870 | p1.(A)5 |
| 1 | 198676006 | 198676010 | p1.(A)5 |
| 1 | 198677301 | 198677305 | p1.(A)5 |
| 1 | 198677333 | 198677337 | p1.(A)5 |
| 1 | 198682103 | 198682108 | p1.(T)6 |
| 1 | 198682150 | 198682154 | p1.(C)5 |
| 1 | 198685800 | 198685804 | p1.(T)5 |
| 1 | 198685811 | 198685815 | p1.(A)5 |
| 1 | 198685834 | 198685838 | p1.(A)5 |
| 1 | 198687311 | 198687315 | p1.(C)5 |
| 1 | 198700752 | 198700756 | p1.(A)5 |
| 1 | 198700836 | 198700840 | p1.(T)5 |
| 1 | 198710998 | 198711003 | p1.(A)6 |
| 1 | 198711005 | 198711009 | p1.(A)5 |
| 1 | 198711161 | 198711165 | p1.(A)5 |
| 1 | 198719615 | 198719619 | p1.(A)5 |
| 1 | 198721829 | 198721833 | p1.(A)5 |
| 1 | 198723478 | 198723482 | p1.(T)5 |
| 1 | 198725101 | 198725105 | p1.(A)5 |
| 1 | 204494595 | 204494603 | p1.(T)9 |
| 1 | 204499948 | 204499952 | p1.(A)5 |
| 1 | 204507366 | 204507370 | p1.(A)5 |
| 1 | 204512004 | 204512008 | p1.(T)5 |
| 1 | 204513648 | 204513655 | p1.(T)8 |
| 1 | 204513708 | 204513712 | p1.(T)5 |
| 1 | 204513807 | 204513811 | p1.(A)5 |
| 1 | 204515939 | 204515944 | p1.(A)6 |
| 1 | 204518491 | 204518495 | p1.(T)5 |
| 1 | 204518797 | 204518801 | p1.(T)5 |
| 1 | 205589099 | 205589105 | p1.(A)7 |
| 1 | 205589395 | 205589399 | p1.(G)5 |
| 1 | 205589581 | 205589585 | p1.(T)5 |
| 1 | 205589637 | 205589642 | p1.(T)6 |
| 1 | 205589952 | 205589957 | p1.(T)6 |
| 1 | 205592873 | 205592877 | p1.(C)5 |
| 1 | 205601104 | 205601109 | p1.(C)6 |
| 1 | 205601160 | 205601164 | p1.(G)5 |
| 1 | 205632214 | 205632218 | p1.(G)5 |
| 1 | 205632349 | 205632353 | p1.(G)5 |
| 1 | 205632404 | 205632408 | p1.(C)5 |
| 1 | 205633643 | 205633647 | p1.(C)5 |
| 1 | 205633809 | 205633813 | p1.(G)5 |
| 1 | 206646621 | 206646625 | p1.(G)5 |
| 1 | 206650064 | 206650068 | p1.(A)5 |
| 1 | 206651502 | 206651506 | p1.(G)5 |
| 1 | 206651689 | 206651693 | p1.(G)5 |
| 1 | 206652330 | 206652335 | p1.(C)6 |
| 1 | 206652424 | 206652428 | p1.(C)5 |
| 1 | 206653438 | 206653442 | p1.(G)5 |
| 1 | 206666428 | 206666432 | p1.(G)5 |
| 1 | 206666643 | 206666647 | p1.(G)5 |
| 1 | 226252006 | 226252010 | p1.(T)5 |
| 1 | 226252013 | 226252017 | p1.(T)5 |
| 1 | 226252186 | 226252191 | p1.(A)6 |
| 1 | 241661228 | 241661232 | p1.(T)5 |
| 1 | 241661277 | 241661282 | p1.(A)6 |
| 1 | 241663854 | 241663858 | p1.(C)5 |
| 1 | 241663883 | 241663887 | p1.(T)5 |
| 1 | 241663894 | 241663905 | p4.(TGAG)3 |
| 1 | 241667416 | 241667420 | p1.(A)5 |
| 1 | 241669471 | 241669485 | p5.(GAAAA)3 |
| 1 | 241675450 | 241675455 | p1.(A)6 |
| 1 | 243663039 | 243663043 | p1.(T)5 |
| 1 | 243663048 | 243663053 | p1.(T)6 |
| 1 | 243675627 | 243675631 | p1.(T)5 |
| 1 | 243675733 | 243675737 | p1.(A)5 |
| 1 | 243708813 | 243708817 | p1.(T)5 |
| 1 | 243716129 | 243716133 | p1.(A)5 |
| 1 | 243727111 | 243727115 | p1.(T)5 |
| 1 | 243778402 | 243778406 | p1.(A)5 |
| 1 | 243801048 | 243801052 | p1.(A)5 |
| 1 | 243809344 | 243809348 | p1.(A)5 |
| 1 | 243859003 | 243859008 | p1.(T)6 |
| 1 | 244006490 | 244006494 | p1.(C)5 |
| 2 | 16082314 | 16082325 | p1.(C)7(G)5 |
| 2 | 16082361 | 16082365 | p1.(C)5 |
| 2 | 16082406 | 16082410 | p1.(C)5 |
| 2 | 16082483 | 16082488 | p1.(G)6 |
| 2 | 16082503 | 16082507 | p1.(C)5 |
| 2 | 16082849 | 16082853 | p1.(G)5 |
| 2 | 16082882 | 16082886 | p1.(G)5 |
| 2 | 16085825 | 16085829 | p1.(C)5 |
| 2 | 16085857 | 16085861 | p1.(C)5 |
| 2 | 16085913 | 16085918 | p1.(C)6 |
| 2 | 16085937 | 16085941 | p1.(C)5 |
| 2 | 16086094 | 16086098 | p1.(A)5 |
| 2 | 16086158 | 16086162 | p1.(A)5 |
| 2 | 24253851 | 24253855 | p1.(G)5 |
| 2 | 24253907 | 24253911 | p1.(A)5 |
| 2 | 24254041 | 24254045 | p1.(A)5 |
| 2 | 24255783 | 24255788 | p1.(T)6 |
| 2 | 24255825 | 24255830 | p1.(A)6 |
| 2 | 24260951 | 24260955 | p1.(A)5 |
| 2 | 24260980 | 24260985 | p1.(T)6 |
| 2 | 24261144 | 24261148 | p1.(T)5 |
| 2 | 24261448 | 24261453 | p1.(T)6 |
| 2 | 24881538 | 24881542 | p1.(T)5 |
| 2 | 24888787 | 24888792 | p1.(A)6 |
| 2 | 24905832 | 24905836 | p1.(T)5 |
| 2 | 24905951 | 24905955 | p1.(G)5 |
| 2 | 24920612 | 24920617 | p1.(T)6 |
| 2 | 24929424 | 24929433 | p1.(T)5(C)5 |
| 2 | 24929917 | 24929928 | p3.(TAA)4 |
| 2 | 24930470 | 24930474 | p1.(A)5 |
| 2 | 24930557 | 24930562 | p1.(A)6 |
| 2 | 24952369 | 24952374 | p1.(G)6 |
| 2 | 24952524 | 24952528 | p1.(A)5 |
| 2 | 24952576 | 24952580 | p1.(T)5 |
| 2 | 24962290 | 24962294 | p1.(T)5 |
| 2 | 24964778 | 24964789 | p4.(CCTC)3 |
| 2 | 24964817 | 24964821 | p1.(C)5 |
| 2 | 24975030 | 24975034 | p1.(G)5 |
| 2 | 24991211 | 24991216 | p1.(C)6 |
| 2 | 25457093 | 25457098 | p1.(T)6 |
| 2 | 25457096 | 25457107 | p4.(TTTG)3 |
| 2 | 25457136 | 25457140 | p1.(C)5 |
| 2 | 25457292 | 25457296 | p1.(G)5 |
| 2 | 25463562 | 25463566 | p1.(C)5 |
| 2 | 25467164 | 25467168 | p1.(C)5 |
| 2 | 25467448 | 25467452 | p1.(C)5 |
| 2 | 25467482 | 25467493 | p3.(CGT)4 |
| 2 | 25468154 | 25468158 | p1.(G)5 |
| 2 | 25469091 | 25469095 | p1.(T)5 |
| 2 | 25469530 | 25469535 | p1.(C)6 |
| 2 | 25470030 | 25470035 | p1.(G)6 |
| 2 | 25470580 | 25470584 | p1.(C)5 |
| 2 | 25470993 | 25470997 | p1.(G)5 |
| 2 | 25497869 | 25497873 | p1.(C)5 |
| 2 | 25505341 | 25505345 | p1.(G)5 |
| 2 | 25505431 | 25505436 | p1.(C)6 |
| 2 | 25523009 | 25523014 | p1.(G)6 |
| 2 | 29416296 | 29416300 | p1.(C)5 |
| 2 | 29416400 | 29416404 | p1.(T)5 |
| 2 | 29416532 | 29416536 | p1.(C)5 |
| 2 | 29416692 | 29416696 | p1.(C)5 |
| 2 | 29430082 | 29430086 | p1.(G)5 |
| 2 | 29443612 | 29443617 | p1.(C)6 |
| 2 | 29445400 | 29445404 | p1.(G)5 |
| 2 | 29446223 | 29446227 | p1.(T)5 |
| 2 | 29449779 | 29449783 | p1.(C)5 |
| 2 | 29451784 | 29451789 | p1.(C)6 |
| 2 | 29451794 | 29451798 | p1.(C)5 |
| 2 | 29451806 | 29451810 | p1.(C)5 |
| 2 | 29451823 | 29451827 | p1.(C)5 |
| 2 | 29451843 | 29451847 | p1.(G)5 |
| 2 | 29456453 | 29456457 | p1.(C)5 |
| 2 | 29474017 | 29474021 | p1.(G)5 |
| 2 | 29497981 | 29497985 | p1.(G)5 |
| 2 | 29543728 | 29543732 | p1.(A)5 |
| 2 | 29940572 | 29940576 | p1.(A)5 |
| 2 | 30143052 | 30143057 | p1.(G)6 |
| 2 | 30143204 | 30143208 | p1.(C)5 |
| 2 | 42472625 | 42472639 | p5.(TATTT)3 |
| 2 | 42472806 | 42472811 | p1.(A)6 |
| 2 | 42509950 | 42509954 | p1.(T)5 |
| 2 | 42511767 | 42511771 | p1.(T)5 |
| 2 | 42513400 | 42513404 | p1.(T)5 |
| 2 | 42513507 | 42513511 | p1.(T)5 |
| 2 | 42522392 | 42522396 | p1.(T)5 |
| 2 | 42528484 | 42528488 | p1.(A)5 |
| 2 | 42530232 | 42530239 | p1.(T)8 |
| 2 | 42530298 | 42530302 | p1.(T)5 |
| 2 | 42557052 | 42557056 | p1.(C)5 |
| 2 | 47630513 | 47630517 | p1.(G)5 |
| 2 | 47635524 | 47635536 | p1.(T)13 |
| 2 | 47637222 | 47637226 | p1.(T)5 |
| 2 | 47641441 | 47641445 | p1.(T)5 |
| 2 | 47641550 | 47641554 | p1.(T)5 |
| 2 | 47641560 | 47641586 | p1.(A)27 |
| 2 | 47657069 | 47657073 | p1.(A)5 |
| 2 | 47672714 | 47672718 | p1.(T)5 |
| 2 | 47693875 | 47693879 | p1.(A)5 |
| 2 | 47693895 | 47693899 | p1.(A)5 |
| 2 | 47702153 | 47702157 | p1.(T)5 |
| 2 | 47702377 | 47702381 | p1.(A)5 |
| 2 | 47702412 | 47702417 | p1.(A)6 |
| 2 | 47705528 | 47705532 | p1.(T)5 |
| 2 | 48010482 | 48010486 | p1.(C)5 |
| 2 | 48010623 | 48010627 | p1.(C)5 |
| 2 | 48018202 | 48018207 | p1.(T)6 |
| 2 | 48023067 | 48023071 | p1.(T)5 |
| 2 | 48025857 | 48025863 | p1.(A)7 |
| 2 | 48025968 | 48025972 | p1.(G)5 |
| 2 | 48026753 | 48026757 | p1.(A)5 |
| 2 | 48026824 | 48026828 | p1.(T)5 |
| 2 | 48026890 | 48026894 | p1.(C)5 |
| 2 | 48026912 | 48026916 | p1.(A)5 |
| 2 | 48027196 | 48027201 | p1.(A)6 |
| 2 | 48027356 | 48027360 | p1.(T)5 |
| 2 | 48027807 | 48027812 | p1.(A)6 |
| 2 | 48028031 | 48028035 | p1.(G)5 |
| 2 | 48028246 | 48028250 | p1.(A)5 |
| 2 | 48030591 | 48030595 | p1.(G)5 |
| 2 | 48030640 | 48030647 | p1.(C)8 |
| 2 | 48030692 | 48030698 | p1.(T)7 |
| 2 | 48030797 | 48030802 | p1.(G)6 |
| 2 | 48032172 | 48032177 | p1.(T)6 |
| 2 | 48032741 | 48032753 | p1.(T)13 |
| 2 | 48032768 | 48032773 | p1.(T)6 |
| 2 | 48033792 | 48033803 | p4.(TAAC)3 |
| 2 | 48034961 | 48034965 | p1.(A)5 |
| 2 | 48035098 | 48035102 | p1.(T)5 |
| 2 | 48035198 | 48035203 | p1.(A)6 |
| 2 | 48035237 | 48035241 | p1.(T)5 |
| 2 | 48035381 | 48035385 | p1.(A)5 |
| 2 | 48035569 | 48035573 | p1.(A)5 |
| 2 | 48036768 | 48036772 | p1.(A)5 |
| 2 | 48036794 | 48036798 | p1.(A)5 |
| 2 | 48040378 | 48040382 | p1.(C)5 |
| 2 | 48045988 | 48045992 | p1.(A)5 |
| 2 | 48047602 | 48047606 | p1.(A)5 |
| 2 | 48050368 | 48050372 | p1.(T)5 |
| 2 | 48050503 | 48050507 | p1.(A)5 |
| 2 | 48059567 | 48059571 | p1.(T)5 |
| 2 | 48059702 | 48059707 | p1.(A)6 |
| 2 | 48059824 | 48059828 | p1.(A)5 |
| 2 | 48060148 | 48060152 | p1.(A)5 |
| 2 | 48065987 | 48065991 | p1.(T)5 |
| 2 | 48066086 | 48066090 | p1.(T)5 |
| 2 | 48066828 | 48066832 | p1.(T)5 |
| 2 | 58392896 | 58392900 | p1.(T)5 |
| 2 | 58392978 | 58392982 | p1.(A)5 |
| 2 | 58393015 | 58393021 | p1.(A)7 |
| 2 | 58425803 | 58425808 | p1.(A)6 |
| 2 | 58456936 | 58456940 | p1.(A)5 |
| 2 | 58468409 | 58468413 | p1.(G)5 |
| 2 | 60687547 | 60687551 | p1.(T)5 |
| 2 | 60687584 | 60687589 | p1.(T)6 |
| 2 | 60688110 | 60688114 | p1.(G)5 |
| 2 | 60688188 | 60688192 | p1.(T)5 |
| 2 | 60688200 | 60688205 | p1.(C)6 |
| 2 | 60688535 | 60688549 | p3.(CTC)5 |
| 2 | 60688748 | 60688752 | p1.(G)5 |
| 2 | 60688929 | 60688933 | p1.(G)5 |
| 2 | 60688969 | 60688975 | p1.(G)7 |
| 2 | 60689197 | 60689201 | p1.(C)5 |
| 2 | 60689218 | 60689222 | p1.(G)5 |
| 2 | 60689254 | 60689260 | p1.(G)7 |
| 2 | 60773209 | 60773214 | p1.(T)6 |
| 2 | 60773254 | 60773258 | p1.(T)5 |
| 2 | 60773302 | 60773306 | p1.(A)5 |
| 2 | 60773316 | 60773320 | p1.(C)5 |
| 2 | 61108902 | 61108913 | p4.(CTGA)3 |
| 2 | 61118800 | 61118809 | p1.(T)5(A)5 |
| 2 | 61121518 | 61121528 | p1.(C)5(T)6 |
| 2 | 61128112 | 61128118 | p1.(T)7 |
| 2 | 61128158 | 61128163 | p1.(A)6 |
| 2 | 61143988 | 61144003 | p1.(A)6(T)10 |
| 2 | 61144018 | 61144022 | p1.(A)5 |
| 2 | 61144078 | 61144082 | p1.(T)5 |
| 2 | 61145665 | 61145669 | p1.(A)5 |
| 2 | 61145736 | 61145740 | p1.(A)5 |
| 2 | 61149057 | 61149071 | p5.(CCCAC)3 |
| 2 | 61149100 | 61149104 | p1.(T)5 |
| 2 | 61149471 | 61149475 | p1.(T)5 |
| 2 | 61149656 | 61149661 | p1.(T)6 |
| 2 | 61705930 | 61705937 | p1.(A)8 |
| 2 | 61706067 | 61706071 | p1.(A)5 |
| 2 | 61709615 | 61709619 | p1.(T)5 |
| 2 | 61711203 | 61711207 | p1.(A)5 |
| 2 | 61711243 | 61711247 | p1.(A)5 |
| 2 | 61712930 | 61712934 | p1.(A)5 |
| 2 | 61712973 | 61712977 | p1.(C)5 |
| 2 | 61713075 | 61713079 | p1.(G)5 |
| 2 | 61715356 | 61715360 | p1.(T)5 |
| 2 | 61715915 | 61715921 | p1.(A)7 |
| 2 | 61717919 | 61717923 | p1.(A)5 |
| 2 | 61719275 | 61719279 | p1.(T)5 |
| 2 | 61719519 | 61719524 | p1.(A)6 |
| 2 | 61719726 | 61719730 | p1.(T)5 |
| 2 | 61719790 | 61719794 | p1.(T)5 |
| 2 | 61719892 | 61719896 | p1.(A)5 |
| 2 | 61722619 | 61722624 | p1.(T)6 |
| 2 | 61725841 | 61725845 | p1.(A)5 |
| 2 | 61729435 | 61729441 | p1.(T)7 |
| 2 | 61729451 | 61729457 | p1.(A)7 |
| 2 | 61749782 | 61749786 | p1.(T)5 |
| 2 | 61760967 | 61760971 | p1.(T)5 |
| 2 | 100167879 | 100167883 | p1.(A)5 |
| 2 | 100168061 | 100168065 | p1.(G)5 |
| 2 | 100170975 | 100170979 | p1.(G)5 |
| 2 | 100171155 | 100171160 | p1.(C)6 |
| 2 | 100176879 | 100176883 | p1.(T)5 |
| 2 | 100176904 | 100176908 | p1.(A)5 |
| 2 | 100199306 | 100199310 | p1.(T)5 |
| 2 | 100199430 | 100199435 | p1.(T)6 |
| 2 | 100199438 | 100199442 | p1.(T)5 |
| 2 | 100199465 | 100199469 | p1.(A)5 |
| 2 | 100209976 | 100209980 | p1.(C)5 |
| 2 | 100210161 | 100210165 | p1.(C)5 |
| 2 | 100210258 | 100210263 | p1.(G)6 |
| 2 | 100217914 | 100217920 | p1.(G)7 |
| 2 | 100218011 | 100218031 | p3.(GCT)7 |
| 2 | 100623096 | 100623100 | p1.(C)5 |
| 2 | 100623488 | 100623499 | p3.(GAA)4 |
| 2 | 100623849 | 100623853 | p1.(A)5 |
| 2 | 100623910 | 100623914 | p1.(C)5 |
| 2 | 100721972 | 100721976 | p1.(G)5 |
| 2 | 111881310 | 111881316 | p1.(A)7 |
| 2 | 111881487 | 111881491 | p1.(C)5 |
| 2 | 111921713 | 111921717 | p1.(T)5 |
| 2 | 113260620 | 113260624 | p1.(A)5 |
| 2 | 113286319 | 113286323 | p1.(C)5 |
| 2 | 113286398 | 113286402 | p1.(A)5 |
| 2 | 113977718 | 113977722 | p1.(G)5 |
| 2 | 113984838 | 113984842 | p1.(A)5 |
| 2 | 113993069 | 113993073 | p1.(A)5 |
| 2 | 113993122 | 113993126 | p1.(G)5 |
| 2 | 113993165 | 113993176 | p4.(GGAG)3 |
| 2 | 113999149 | 113999153 | p1.(G)5 |
| 2 | 113999254 | 113999265 | p3.(GCT)4 |
| 2 | 113999310 | 113999315 | p1.(C)6 |
| 2 | 113999683 | 113999687 | p1.(G)5 |
| 2 | 113999715 | 113999719 | p1.(G)5 |
| 2 | 114002135 | 114002139 | p1.(G)5 |
| 2 | 114002160 | 114002164 | p1.(C)5 |
| 2 | 128018892 | 128018896 | p1.(A)5 |
| 2 | 128018930 | 128018935 | p1.(A)6 |
| 2 | 128028913 | 128028918 | p1.(T)6 |
| 2 | 128030506 | 128030510 | p1.(C)5 |
| 2 | 128046254 | 128046258 | p1.(C)5 |
| 2 | 128046944 | 128046958 | p3.(TCT)5 |
| 2 | 140992437 | 140992441 | p1.(C)5 |
| 2 | 140995858 | 140995862 | p1.(T)5 |
| 2 | 140997015 | 140997019 | p1.(T)5 |
| 2 | 141004734 | 141004738 | p1.(A)5 |
| 2 | 141032034 | 141032039 | p1.(C)6 |
| 2 | 141032173 | 141032179 | p1.(A)7 |
| 2 | 141055467 | 141055471 | p1.(A)5 |
| 2 | 141081550 | 141081554 | p1.(T)5 |
| 2 | 141081572 | 141081576 | p1.(T)5 |
| 2 | 141108389 | 141108394 | p1.(T)6 |
| 2 | 141110564 | 141110573 | p2.(CA)5 |
| 2 | 141113925 | 141113929 | p1.(T)5 |
| 2 | 141113974 | 141113978 | p1.(T)5 |
| 2 | 141115624 | 141115629 | p1.(T)6 |
| 2 | 141115652 | 141115656 | p1.(T)5 |
| 2 | 141116522 | 141116526 | p1.(A)5 |
| 2 | 141128266 | 141128270 | p1.(A)5 |
| 2 | 141128365 | 141128369 | p1.(T)5 |
| 2 | 141130712 | 141130716 | p1.(A)5 |
| 2 | 141135864 | 141135870 | p1.(A)7 |
| 2 | 141143539 | 141143543 | p1.(T)5 |
| 2 | 141143572 | 141143576 | p1.(T)5 |
| 2 | 141208234 | 141208239 | p1.(A)6 |
| 2 | 141214104 | 141214108 | p1.(T)5 |
| 2 | 141242901 | 141242905 | p1.(T)5 |
| 2 | 141243012 | 141243017 | p1.(T)6 |
| 2 | 141243038 | 141243043 | p1.(T)6 |
| 2 | 141259407 | 141259411 | p1.(T)5 |
| 2 | 141259418 | 141259423 | p1.(A)6 |
| 2 | 141259448 | 141259455 | p1.(A)8 |
| 2 | 141272298 | 141272302 | p1.(T)5 |
| 2 | 141283924 | 141283928 | p1.(A)5 |
| 2 | 141294163 | 141294167 | p1.(T)5 |
| 2 | 141294272 | 141294276 | p1.(T)5 |
| 2 | 141294286 | 141294298 | p1.(A)13 |
| 2 | 141299414 | 141299418 | p1.(T)5 |
| 2 | 141356235 | 141356239 | p1.(T)5 |
| 2 | 141356336 | 141356340 | p1.(T)5 |
| 2 | 141359214 | 141359218 | p1.(G)5 |
| 2 | 141458023 | 141458027 | p1.(T)5 |
| 2 | 141459420 | 141459424 | p1.(A)5 |
| 2 | 141459696 | 141459701 | p1.(A)6 |
| 2 | 141597545 | 141597552 | p1.(A)8 |
| 2 | 141598486 | 141598490 | p1.(C)5 |
| 2 | 141607888 | 141607893 | p1.(T)6 |
| 2 | 141607901 | 141607906 | p1.(A)6 |
| 2 | 141609260 | 141609264 | p1.(G)5 |
| 2 | 141660739 | 141660749 | p1.(A)5(G)6 |
| 2 | 141665484 | 141665488 | p1.(T)5 |
| 2 | 141680595 | 141680599 | p1.(T)5 |
| 2 | 141680622 | 141680626 | p1.(T)5 |
| 2 | 141751647 | 141751651 | p1.(T)5 |
| 2 | 141751713 | 141751719 | p1.(A)7 |
| 2 | 141762996 | 141763000 | p1.(C)5 |
| 2 | 141771114 | 141771119 | p1.(T)6 |
| 2 | 141771219 | 141771223 | p1.(A)5 |
| 2 | 141773284 | 141773288 | p1.(A)5 |
| 2 | 141773292 | 141773296 | p1.(T)5 |
| 2 | 141773388 | 141773392 | p1.(A)5 |
| 2 | 141773405 | 141773416 | p4.(ATCC)3 |
| 2 | 141812783 | 141812787 | p1.(C)5 |
| 2 | 141816467 | 141816472 | p1.(T)6 |
| 2 | 141819648 | 141819653 | p1.(T)6 |
| 2 | 142004926 | 142004930 | p1.(A)5 |
| 2 | 142012219 | 142012224 | p1.(A)6 |
| 2 | 142888355 | 142888372 | p3.(CGG)6 |
| 2 | 173429358 | 173429362 | p1.(T)5 |
| 2 | 173429695 | 173429699 | p1.(T)5 |
| 2 | 173435501 | 173435506 | p1.(C)6 |
| 2 | 173460549 | 173460553 | p1.(T)5 |
| 2 | 175664838 | 175664842 | p1.(A)5 |
| 2 | 175664863 | 175664867 | p1.(T)5 |
| 2 | 175665019 | 175665023 | p1.(A)5 |
| 2 | 175676229 | 175676233 | p1.(A)5 |
| 2 | 175676320 | 175676324 | p1.(A)5 |
| 2 | 175677132 | 175677136 | p1.(T)5 |
| 2 | 175689250 | 175689260 | p1.(A)11 |
| 2 | 175742640 | 175742645 | p1.(T)6 |
| 2 | 176957811 | 176957825 | p3.(GCG)5 |
| 2 | 176957922 | 176957926 | p1.(C)5 |
| 2 | 176957973 | 176957977 | p1.(C)5 |
| 2 | 176972310 | 176972327 | p3.(GCG)6 |
| 2 | 176972335 | 176972340 | p1.(G)6 |
| 2 | 176972351 | 176972355 | p1.(G)5 |
| 2 | 176972366 | 176972370 | p1.(G)5 |
| 2 | 176972369 | 176972380 | p3.(GGC)4 |
| 2 | 176972383 | 176972388 | p1.(G)6 |
| 2 | 176972405 | 176972422 | p3.(GCG)6 |
| 2 | 176973638 | 176973643 | p1.(C)6 |
| 2 | 176973655 | 176973659 | p1.(A)5 |
| 2 | 176973706 | 176973710 | p1.(T)5 |
| 2 | 178095517 | 178095521 | p1.(T)5 |
| 2 | 178095666 | 178095671 | p1.(T)6 |
| 2 | 178095702 | 178095706 | p1.(T)5 |
| 2 | 178095717 | 178095721 | p1.(T)5 |
| 2 | 178095723 | 178095727 | p1.(T)5 |
| 2 | 178095782 | 178095786 | p1.(T)5 |
| 2 | 178095914 | 178095919 | p1.(T)6 |
| 2 | 178096620 | 178096624 | p1.(T)5 |
| 2 | 178096646 | 178096650 | p1.(A)5 |
| 2 | 178098833 | 178098837 | p1.(A)5 |
| 2 | 178098886 | 178098891 | p1.(T)6 |
| 2 | 178098895 | 178098899 | p1.(T)5 |
| 2 | 190660590 | 190660594 | p1.(A)5 |
| 2 | 190670364 | 190670375 | p1.(T)7(C)5 |
| 2 | 190708677 | 190708681 | p1.(T)5 |
| 2 | 190717487 | 190717491 | p1.(A)5 |
| 2 | 190718958 | 190718962 | p1.(T)5 |
| 2 | 190719104 | 190719108 | p1.(T)5 |
| 2 | 190719198 | 190719203 | p1.(A)6 |
| 2 | 190719286 | 190719290 | p1.(A)5 |
| 2 | 190719511 | 190719515 | p1.(A)5 |
| 2 | 190719560 | 190719565 | p1.(A)6 |
| 2 | 190719640 | 190719645 | p1.(A)6 |
| 2 | 190719737 | 190719741 | p1.(T)5 |
| 2 | 190719845 | 190719849 | p1.(A)5 |
| 2 | 190732517 | 190732521 | p1.(T)5 |
| 2 | 190732527 | 190732531 | p1.(T)5 |
| 2 | 190738207 | 190738214 | p1.(T)8 |
| 2 | 190742124 | 190742129 | p1.(T)6 |
| 2 | 191897673 | 191897677 | p1.(A)5 |
| 2 | 191897880 | 191897884 | p1.(A)5 |
| 2 | 191898281 | 191898285 | p1.(A)5 |
| 2 | 191898355 | 191898359 | p1.(A)5 |
| 2 | 191904031 | 191904042 | p1.(G)6(A)6 |
| 2 | 191926444 | 191926448 | p1.(T)5 |
| 2 | 191929569 | 191929573 | p1.(C)5 |
| 2 | 191929687 | 191929691 | p1.(A)5 |
| 2 | 191937834 | 191937838 | p1.(T)5 |
| 2 | 191937921 | 191937925 | p1.(A)5 |
| 2 | 192011359 | 192011363 | p1.(T)5 |
| 2 | 192011385 | 192011389 | p1.(T)5 |
| 2 | 192011486 | 192011496 | p1.(A)11 |
| 2 | 192012886 | 192012890 | p1.(A)5 |
| 2 | 198261056 | 198261062 | p1.(A)7 |
| 2 | 198262763 | 198262767 | p1.(T)5 |
| 2 | 198262848 | 198262853 | p1.(A)6 |
| 2 | 198263229 | 198263233 | p1.(T)5 |
| 2 | 198263253 | 198263257 | p1.(G)5 |
| 2 | 198265161 | 198265166 | p1.(A)6 |
| 2 | 198265558 | 198265562 | p1.(T)5 |
| 2 | 198265663 | 198265667 | p1.(A)5 |
| 2 | 198266175 | 198266180 | p1.(A)6 |
| 2 | 198268494 | 198268498 | p1.(A)5 |
| 2 | 198269829 | 198269833 | p1.(T)5 |
| 2 | 198269850 | 198269854 | p1.(T)5 |
| 2 | 198269860 | 198269864 | p1.(T)5 |
| 2 | 198269909 | 198269913 | p1.(A)5 |
| 2 | 198270036 | 198270040 | p1.(A)5 |
| 2 | 198274507 | 198274511 | p1.(G)5 |
| 2 | 198274526 | 198274530 | p1.(T)5 |
| 2 | 198274641 | 198274645 | p1.(T)5 |
| 2 | 198274735 | 198274740 | p1.(A)6 |
| 2 | 198281452 | 198281456 | p1.(A)5 |
| 2 | 198281489 | 198281494 | p1.(T)6 |
| 2 | 198281606 | 198281610 | p1.(T)5 |
| 2 | 198281639 | 198281645 | p1.(A)7 |
| 2 | 198285205 | 198285209 | p1.(T)5 |
| 2 | 198288703 | 198288707 | p1.(A)5 |
| 2 | 202131204 | 202131208 | p1.(A)5 |
| 2 | 202149728 | 202149732 | p1.(C)5 |
| 2 | 202149799 | 202149804 | p1.(C)6 |
| 2 | 202149819 | 202149823 | p1.(G)5 |
| 2 | 202151246 | 202151250 | p1.(A)5 |
| 2 | 202151290 | 202151296 | p1.(A)7 |
| 2 | 202151331 | 202151350 | p5.(TTTGT)4 |
| 2 | 204732741 | 204732746 | p1.(T)6 |
| 2 | 204736166 | 204736172 | p1.(T)7 |
| 2 | 204737476 | 204737480 | p1.(C)5 |
| 2 | 204737596 | 204737601 | p1.(T)6 |
| 2 | 208442219 | 208442225 | p1.(T)7 |
| 2 | 209101739 | 209101743 | p1.(A)5 |
| 2 | 209103947 | 209103951 | p1.(A)5 |
| 2 | 209106756 | 209106760 | p1.(T)5 |
| 2 | 209108211 | 209108215 | p1.(T)5 |
| 2 | 209113152 | 209113156 | p1.(G)5 |
| 2 | 209113160 | 209113164 | p1.(T)5 |
| 2 | 209116180 | 209116184 | p1.(A)5 |
| 2 | 209116263 | 209116269 | p1.(T)7 |
| 2 | 209116294 | 209116298 | p1.(A)5 |
| 2 | 209116300 | 209116309 | p1.(A)10 |
| 2 | 212248322 | 212248327 | p1.(A)6 |
| 2 | 212248465 | 212248469 | p1.(A)5 |
| 2 | 212248737 | 212248742 | p1.(T)6 |
| 2 | 212251753 | 212251757 | p1.(A)5 |
| 2 | 212295694 | 212295698 | p1.(T)5 |
| 2 | 212295829 | 212295833 | p1.(A)5 |
| 2 | 212426821 | 212426827 | p1.(A)7 |
| 2 | 212484009 | 212484015 | p1.(A)7 |
| 2 | 212488635 | 212488640 | p1.(T)6 |
| 2 | 212495221 | 212495225 | p1.(T)5 |
| 2 | 212530121 | 212530125 | p1.(C)5 |
| 2 | 212530143 | 212530147 | p1.(T)5 |
| 2 | 212537933 | 212537937 | p1.(G)5 |
| 2 | 212566899 | 212566903 | p1.(G)5 |
| 2 | 212568866 | 212568871 | p1.(A)6 |
| 2 | 212570033 | 212570038 | p1.(A)6 |
| 2 | 212578380 | 212578393 | p1.(A)14 |
| 2 | 212652892 | 212652897 | p1.(A)6 |
| 2 | 212812214 | 212812218 | p1.(A)5 |
| 2 | 212989591 | 212989600 | p2.(AG)5 |
| 2 | 213403254 | 213403259 | p1.(T)6 |
| 2 | 213403307 | 213403311 | p1.(C)5 |
| 2 | 215593635 | 215593639 | p1.(C)5 |
| 2 | 215595127 | 215595131 | p1.(A)5 |
| 2 | 215595238 | 215595242 | p1.(A)5 |
| 2 | 215610468 | 215610473 | p1.(T)6 |
| 2 | 215634041 | 215634045 | p1.(A)5 |
| 2 | 215645459 | 215645463 | p1.(T)5 |
| 2 | 215645681 | 215645685 | p1.(T)5 |
| 2 | 215645926 | 215645930 | p1.(T)5 |
| 2 | 215645947 | 215645951 | p1.(T)5 |
| 2 | 215645975 | 215645981 | p1.(T)7 |
| 2 | 215645984 | 215645989 | p1.(T)6 |
| 2 | 215646008 | 215646012 | p1.(T)5 |
| 2 | 215646085 | 215646091 | p1.(T)7 |
| 2 | 215646241 | 215646247 | p1.(A)7 |
| 2 | 216182872 | 216182876 | p1.(T)5 |
| 2 | 216182916 | 216182921 | p1.(G)6 |
| 2 | 216184375 | 216184380 | p1.(T)6 |
| 2 | 216184457 | 216184461 | p1.(A)5 |
| 2 | 216190739 | 216190743 | p1.(A)5 |
| 2 | 216191536 | 216191540 | p1.(T)5 |
| 2 | 216197092 | 216197096 | p1.(T)5 |
| 2 | 216199709 | 216199713 | p1.(A)5 |
| 2 | 216203619 | 216203623 | p1.(A)5 |
| 2 | 216214252 | 216214256 | p1.(C)5 |
| 2 | 216214394 | 216214399 | p1.(T)6 |
| 2 | 219846402 | 219846407 | p1.(C)6 |
| 2 | 219846447 | 219846451 | p1.(G)5 |
| 2 | 219848978 | 219848982 | p1.(C)5 |
| 2 | 223066018 | 223066025 | p1.(C)8 |
| 2 | 223066830 | 223066834 | p1.(C)5 |
| 2 | 223066912 | 223066918 | p1.(A)7 |
| 2 | 223085082 | 223085086 | p1.(A)5 |
| 2 | 223086020 | 223086025 | p1.(C)6 |
| 2 | 223159023 | 223159027 | p1.(G)5 |
| 2 | 223161883 | 223161887 | p1.(A)5 |
| 2 | 223163338 | 223163342 | p1.(G)5 |
| 2 | 223787801 | 223787805 | p1.(A)5 |
| 2 | 223787808 | 223787813 | p1.(A)6 |
| 2 | 223789195 | 223789199 | p1.(A)5 |
| 2 | 223791722 | 223791728 | p1.(T)7 |
| 2 | 223791766 | 223791770 | p1.(G)5 |
| 2 | 223793638 | 223793643 | p1.(A)6 |
| 2 | 223795389 | 223795393 | p1.(G)5 |
| 2 | 223795419 | 223795423 | p1.(A)5 |
| 2 | 223795430 | 223795434 | p1.(A)5 |
| 2 | 223797889 | 223797893 | p1.(A)5 |
| 2 | 223806243 | 223806247 | p1.(A)5 |
| 2 | 237489563 | 237489567 | p1.(C)5 |
| 2 | 242793291 | 242793295 | p1.(G)5 |
| 2 | 242793363 | 242793368 | p1.(G)6 |
| 2 | 242794819 | 242794823 | p1.(G)5 |
| 2 | 242795104 | 242795110 | p1.(G)7 |
| 3 | 9027284 | 9027298 | p3.(GCT)5 |
| 3 | 9027335 | 9027340 | p1.(G)6 |
| 3 | 9032370 | 9032374 | p1.(C)5 |
| 3 | 9032381 | 9032385 | p1.(G)5 |
| 3 | 9032393 | 9032397 | p1.(G)5 |
| 3 | 9032473 | 9032477 | p1.(C)5 |
| 3 | 9034600 | 9034606 | p1.(C)7 |
| 3 | 9034634 | 9034638 | p1.(G)5 |
| 3 | 9034650 | 9034654 | p1.(T)5 |
| 3 | 9036052 | 9036056 | p1.(G)5 |
| 3 | 9036130 | 9036134 | p1.(C)5 |
| 3 | 9051979 | 9051983 | p1.(C)5 |
| 3 | 9055018 | 9055022 | p1.(T)5 |
| 3 | 9055047 | 9055051 | p1.(G)5 |
| 3 | 9057404 | 9057408 | p1.(G)5 |
| 3 | 9066959 | 9066963 | p1.(T)5 |
| 3 | 9067025 | 9067030 | p1.(A)6 |
| 3 | 9074419 | 9074423 | p1.(G)5 |
| 3 | 9074430 | 9074435 | p1.(G)6 |
| 3 | 9099940 | 9099944 | p1.(C)5 |
| 3 | 9106069 | 9106073 | p1.(C)5 |
| 3 | 9106268 | 9106272 | p1.(G)5 |
| 3 | 9146365 | 9146369 | p1.(T)5 |
| 3 | 9166511 | 9166515 | p1.(A)5 |
| 3 | 10074539 | 10074543 | p1.(A)5 |
| 3 | 10076843 | 10076852 | p1.(T)10 |
| 3 | 10077963 | 10077968 | p1.(T)6 |
| 3 | 10078012 | 10078018 | p1.(T)7 |
| 3 | 10084236 | 10084240 | p1.(T)5 |
| 3 | 10089712 | 10089717 | p1.(T)6 |
| 3 | 10094059 | 10094064 | p1.(T)6 |
| 3 | 10106400 | 10106404 | p1.(T)5 |
| 3 | 10114640 | 10114644 | p1.(A)5 |
| 3 | 10114658 | 10114662 | p1.(A)5 |
| 3 | 10116267 | 10116272 | p1.(T)6 |
| 3 | 10119789 | 10119793 | p1.(C)5 |
| 3 | 10122854 | 10122858 | p1.(T)5 |
| 3 | 10123017 | 10123021 | p1.(T)5 |
| 3 | 10123023 | 10123027 | p1.(T)5 |
| 3 | 10123127 | 10123131 | p1.(T)5 |
| 3 | 10123139 | 10123143 | p1.(T)5 |
| 3 | 10128940 | 10128945 | p1.(A)6 |
| 3 | 10138052 | 10138056 | p1.(A)5 |
| 3 | 10138138 | 10138143 | p1.(A)6 |
| 3 | 10140655 | 10140659 | p1.(T)5 |
| 3 | 10188297 | 10188301 | p1.(T)5 |
| 3 | 12421190 | 12421194 | p1.(T)5 |
| 3 | 12458270 | 12458275 | p1.(C)6 |
| 3 | 12626352 | 12626356 | p1.(A)5 |
| 3 | 12626656 | 12626660 | p1.(C)5 |
| 3 | 12632476 | 12632490 | p5.(GGGGA)3 |
| 3 | 12641658 | 12641662 | p1.(T)5 |
| 3 | 12641708 | 12641712 | p1.(G)5 |
| 3 | 14188875 | 14188879 | p1.(T)5 |
| 3 | 14188888 | 14188892 | p1.(A)5 |
| 3 | 14190333 | 14190337 | p1.(G)5 |
| 3 | 14199816 | 14199821 | p1.(T)6 |
| 3 | 14199970 | 14199974 | p1.(G)5 |
| 3 | 14214448 | 14214453 | p1.(T)6 |
| 3 | 14214472 | 14214476 | p1.(C)5 |
| 3 | 14214538 | 14214542 | p1.(G)5 |
| 3 | 14219966 | 14219980 | p3.(CCT)5 |
| 3 | 30648341 | 30648345 | p1.(G)5 |
| 3 | 30648383 | 30648387 | p1.(G)5 |
| 3 | 30713742 | 30713746 | p1.(G)5 |
| 3 | 30715585 | 30715589 | p1.(T)5 |
| 3 | 37035101 | 37035105 | p1.(G)5 |
| 3 | 37050315 | 37050320 | p1.(T)6 |
| 3 | 37050349 | 37050354 | p1.(A)6 |
| 3 | 37053348 | 37053353 | p1.(A)6 |
| 3 | 37056043 | 37056047 | p1.(A)5 |
| 3 | 37059062 | 37059066 | p1.(A)5 |
| 3 | 37067100 | 37067120 | p1.(T)21 |
| 3 | 37067416 | 37067420 | p1.(A)5 |
| 3 | 37067433 | 37067437 | p1.(G)5 |
| 3 | 37070349 | 37070354 | p1.(C)6 |
| 3 | 37083746 | 37083750 | p1.(T)5 |
| 3 | 37090053 | 37090057 | p1.(C)5 |
| 3 | 38181482 | 38181486 | p1.(C)5 |
| 3 | 38182798 | 38182807 | p2.(GT)5 |
| 3 | 41265503 | 41265509 | p1.(T)7 |
| 3 | 41266867 | 41266871 | p1.(A)5 |
| 3 | 41268677 | 41268690 | p2.(AT)7 |
| 3 | 41268763 | 41268767 | p1.(A)5 |
| 3 | 41275622 | 41275626 | p1.(T)5 |
| 3 | 41277217 | 41277221 | p1.(G)5 |
| 3 | 41277997 | 41278001 | p1.(A)5 |
| 3 | 47061320 | 47061324 | p1.(T)5 |
| 3 | 47061333 | 47061337 | p1.(A)5 |
| 3 | 47084127 | 47084131 | p1.(T)5 |
| 3 | 47084146 | 47084151 | p1.(G)6 |
| 3 | 47098488 | 47098492 | p1.(G)5 |
| 3 | 47098957 | 47098961 | p1.(T)5 |
| 3 | 47103666 | 47103670 | p1.(T)5 |
| 3 | 47103755 | 47103764 | p2.(TC)5 |
| 3 | 47125832 | 47125836 | p1.(T)5 |
| 3 | 47127812 | 47127816 | p1.(G)5 |
| 3 | 47129682 | 47129686 | p1.(T)5 |
| 3 | 47143010 | 47143015 | p1.(A)6 |
| 3 | 47144836 | 47144840 | p1.(T)5 |
| 3 | 47144893 | 47144897 | p1.(T)5 |
| 3 | 47147524 | 47147528 | p1.(T)5 |
| 3 | 47155352 | 47155357 | p1.(A)6 |
| 3 | 47161807 | 47161811 | p1.(G)5 |
| 3 | 47161907 | 47161913 | p1.(T)7 |
| 3 | 47161939 | 47161945 | p1.(T)7 |
| 3 | 47161951 | 47161956 | p1.(T)6 |
| 3 | 47161958 | 47161962 | p1.(A)5 |
| 3 | 47162078 | 47162082 | p1.(A)5 |
| 3 | 47162414 | 47162418 | p1.(A)5 |
| 3 | 47162429 | 47162433 | p1.(T)5 |
| 3 | 47162731 | 47162735 | p1.(A)5 |
| 3 | 47162778 | 47162782 | p1.(T)5 |
| 3 | 47163364 | 47163368 | p1.(A)5 |
| 3 | 47163377 | 47163383 | p1.(T)7 |
| 3 | 47163936 | 47163940 | p1.(T)5 |
| 3 | 47164127 | 47164131 | p1.(A)5 |
| 3 | 47164218 | 47164222 | p1.(A)5 |
| 3 | 47164240 | 47164245 | p1.(T)6 |
| 3 | 47164309 | 47164313 | p1.(T)5 |
| 3 | 47164414 | 47164418 | p1.(T)5 |
| 3 | 47164525 | 47164529 | p1.(G)5 |
| 3 | 47165048 | 47165052 | p1.(T)5 |
| 3 | 47165068 | 47165072 | p1.(T)5 |
| 3 | 47165213 | 47165219 | p1.(T)7 |
| 3 | 47165283 | 47165289 | p1.(T)7 |
| 3 | 47165962 | 47165966 | p1.(A)5 |
| 3 | 47205375 | 47205379 | p1.(C)5 |
| 3 | 47205443 | 47205447 | p1.(C)5 |
| 3 | 47205468 | 47205472 | p1.(G)5 |
| 3 | 47205469 | 47205483 | p5.(GGGGA)3 |
| 3 | 48711991 | 48711995 | p1.(C)5 |
| 3 | 48716309 | 48716313 | p1.(C)5 |
| 3 | 48716893 | 48716899 | p1.(G)7 |
| 3 | 48719000 | 48719004 | p1.(G)5 |
| 3 | 48719030 | 48719034 | p1.(G)5 |
| 3 | 48719081 | 48719085 | p1.(G)5 |
| 3 | 48719186 | 48719190 | p1.(C)5 |
| 3 | 48719525 | 48719529 | p1.(G)5 |
| 3 | 48720448 | 48720455 | p1.(G)8 |
| 3 | 52437565 | 52437569 | p1.(A)5 |
| 3 | 52437697 | 52437701 | p1.(G)5 |
| 3 | 52439132 | 52439136 | p1.(G)5 |
| 3 | 52439770 | 52439775 | p1.(C)6 |
| 3 | 52440912 | 52440917 | p1.(C)6 |
| 3 | 52441339 | 52441343 | p1.(A)5 |
| 3 | 52442625 | 52442629 | p1.(G)5 |
| 3 | 52443613 | 52443617 | p1.(C)5 |
| 3 | 52443950 | 52443954 | p1.(C)5 |
| 3 | 52584541 | 52584545 | p1.(G)5 |
| 3 | 52584583 | 52584587 | p1.(G)5 |
| 3 | 52588798 | 52588803 | p1.(C)6 |
| 3 | 52597513 | 52597518 | p1.(A)6 |
| 3 | 52610609 | 52610614 | p1.(T)6 |
| 3 | 52610623 | 52610627 | p1.(T)5 |
| 3 | 52610674 | 52610678 | p1.(A)5 |
| 3 | 52610705 | 52610709 | p1.(T)5 |
| 3 | 52613210 | 52613214 | p1.(T)5 |
| 3 | 52620657 | 52620661 | p1.(A)5 |
| 3 | 52620707 | 52620712 | p1.(G)6 |
| 3 | 52621438 | 52621442 | p1.(A)5 |
| 3 | 52621447 | 52621451 | p1.(T)5 |
| 3 | 52623187 | 52623191 | p1.(T)5 |
| 3 | 52623279 | 52623285 | p1.(A)7 |
| 3 | 52637540 | 52637545 | p1.(T)6 |
| 3 | 52637589 | 52637593 | p1.(T)5 |
| 3 | 52637671 | 52637675 | p1.(T)5 |
| 3 | 52637700 | 52637705 | p1.(A)6 |
| 3 | 52643538 | 52643542 | p1.(A)5 |
| 3 | 52643771 | 52643776 | p1.(T)6 |
| 3 | 52643791 | 52643795 | p1.(T)5 |
| 3 | 52643943 | 52643948 | p1.(T)6 |
| 3 | 52649393 | 52649404 | p3.(TCA)4 |
| 3 | 52651438 | 52651442 | p1.(T)5 |
| 3 | 52651549 | 52651553 | p1.(T)5 |
| 3 | 52651562 | 52651566 | p1.(G)5 |
| 3 | 52661290 | 52661294 | p1.(T)5 |
| 3 | 52668671 | 52668676 | p1.(T)6 |
| 3 | 52668694 | 52668698 | p1.(A)5 |
| 3 | 52668760 | 52668764 | p1.(A)5 |
| 3 | 52677254 | 52677258 | p1.(T)5 |
| 3 | 52682400 | 52682405 | p1.(T)6 |
| 3 | 52692236 | 52692240 | p1.(A)5 |
| 3 | 52702538 | 52702542 | p1.(A)5 |
| 3 | 52702591 | 52702595 | p1.(T)5 |
| 3 | 53529193 | 53529213 | p3.(GAT)7 |
| 3 | 53529215 | 53529221 | p1.(A)7 |
| 3 | 53531331 | 53531335 | p1.(C)5 |
| 3 | 53531385 | 53531390 | p1.(A)6 |
| 3 | 53531495 | 53531506 | p1.(T)7(G)5 |
| 3 | 53535633 | 53535638 | p1.(T)6 |
| 3 | 53535671 | 53535675 | p1.(T)5 |
| 3 | 53684793 | 53684797 | p1.(T)5 |
| 3 | 53684807 | 53684811 | p1.(A)5 |
| 3 | 53684834 | 53684838 | p1.(T)5 |
| 3 | 53684851 | 53684855 | p1.(T)5 |
| 3 | 53699795 | 53699799 | p1.(T)5 |
| 3 | 53699805 | 53699809 | p1.(A)5 |
| 3 | 53699823 | 53699829 | p1.(T)7 |
| 3 | 53707806 | 53707810 | p1.(T)5 |
| 3 | 53757510 | 53757514 | p1.(A)5 |
| 3 | 53757896 | 53757900 | p1.(T)5 |
| 3 | 53760960 | 53760964 | p1.(G)5 |
| 3 | 53764570 | 53764574 | p1.(A)5 |
| 3 | 53766932 | 53766937 | p1.(A)6 |
| 3 | 53766941 | 53766945 | p1.(C)5 |
| 3 | 53783459 | 53783463 | p1.(A)5 |
| 3 | 53783488 | 53783492 | p1.(A)5 |
| 3 | 53785830 | 53785834 | p1.(A)5 |
| 3 | 53785937 | 53785941 | p1.(A)5 |
| 3 | 53804484 | 53804488 | p1.(T)5 |
| 3 | 53804530 | 53804535 | p1.(G)6 |
| 3 | 53809907 | 53809911 | p1.(A)5 |
| 3 | 53810048 | 53810052 | p1.(T)5 |
| 3 | 53810622 | 53810626 | p1.(T)5 |
| 3 | 53810766 | 53810770 | p1.(A)5 |
| 3 | 53815579 | 53815584 | p1.(T)6 |
| 3 | 53834368 | 53834379 | p3.(AGA)4 |
| 3 | 53837522 | 53837526 | p1.(C)5 |
| 3 | 53837534 | 53837538 | p1.(G)5 |
| 3 | 53842755 | 53842759 | p1.(C)5 |
| 3 | 53844126 | 53844131 | p1.(C)6 |
| 3 | 53844281 | 53844285 | p1.(A)5 |
| 3 | 53845307 | 53845311 | p1.(C)5 |
| 3 | 53845434 | 53845438 | p1.(C)5 |
| 3 | 59999769 | 59999773 | p1.(T)5 |
| 3 | 69987193 | 69987197 | p1.(A)5 |
| 3 | 69988240 | 69988244 | p1.(T)5 |
| 3 | 69990375 | 69990379 | p1.(T)5 |
| 3 | 69998250 | 69998254 | p1.(C)5 |
| 3 | 69998303 | 69998307 | p1.(A)5 |
| 3 | 70000969 | 70000973 | p1.(T)5 |
| 3 | 70001016 | 70001020 | p1.(A)5 |
| 3 | 70014243 | 70014247 | p1.(A)5 |
| 3 | 70014329 | 70014333 | p1.(C)5 |
| 3 | 71007357 | 71007371 | p5.(AAAAG)3 |
| 3 | 71007372 | 71007378 | p1.(A)7 |
| 3 | 71007398 | 71007402 | p1.(C)5 |
| 3 | 71007409 | 71007413 | p1.(C)5 |
| 3 | 71007463 | 71007472 | p1.(T)10 |
| 3 | 71008342 | 71008354 | p1.(T)13 |
| 3 | 71008357 | 71008361 | p1.(T)5 |
| 3 | 71008547 | 71008552 | p1.(A)6 |
| 3 | 71019936 | 71019940 | p1.(T)5 |
| 3 | 71021729 | 71021733 | p1.(T)5 |
| 3 | 71027087 | 71027092 | p1.(G)6 |
| 3 | 71027189 | 71027195 | p1.(A)7 |
| 3 | 71064782 | 71064786 | p1.(G)5 |
| 3 | 71101710 | 71101721 | p3.(TGT)4 |
| 3 | 71101751 | 71101756 | p1.(T)6 |
| 3 | 71102805 | 71102816 | p3.(CTG)4 |
| 3 | 71161797 | 71161801 | p1.(A)5 |
| 3 | 71247357 | 71247368 | p3.(TGC)4 |
| 3 | 71247502 | 71247506 | p1.(T)5 |
| 3 | 71247538 | 71247542 | p1.(A)5 |
| 3 | 89259454 | 89259458 | p1.(A)5 |
| 3 | 89391024 | 89391029 | p1.(A)6 |
| 3 | 89457252 | 89457257 | p1.(A)6 |
| 3 | 89499363 | 89499368 | p1.(C)6 |
| 3 | 89521599 | 89521606 | p1.(T)8 |
| 3 | 89521775 | 89521783 | p1.(A)9 |
| 3 | 89528536 | 89528542 | p1.(T)7 |
| 3 | 89528554 | 89528558 | p1.(A)5 |
| 3 | 100447544 | 100447548 | p1.(T)5 |
| 3 | 100451343 | 100451347 | p1.(T)5 |
| 3 | 100451449 | 100451454 | p1.(A)6 |
| 3 | 100451471 | 100451475 | p1.(A)5 |
| 3 | 100463652 | 100463669 | p2.(TG)9 |
| 3 | 105377957 | 105377961 | p1.(T)5 |
| 3 | 105378046 | 105378050 | p1.(G)5 |
| 3 | 105389200 | 105389204 | p1.(A)5 |
| 3 | 105397288 | 105397292 | p1.(A)5 |
| 3 | 105397370 | 105397374 | p1.(G)5 |
| 3 | 105400444 | 105400448 | p1.(A)5 |
| 3 | 105470321 | 105470325 | p1.(A)5 |
| 3 | 105470336 | 105470340 | p1.(A)5 |
| 3 | 105495229 | 105495233 | p1.(T)5 |
| 3 | 105495248 | 105495253 | p1.(A)6 |
| 3 | 105572327 | 105572331 | p1.(T)5 |
| 3 | 105572471 | 105572475 | p1.(T)5 |
| 3 | 105572511 | 105572515 | p1.(A)5 |
| 3 | 105586315 | 105586319 | p1.(G)5 |
| 3 | 119545603 | 119545607 | p1.(T)5 |
| 3 | 119562124 | 119562128 | p1.(G)5 |
| 3 | 119582342 | 119582347 | p1.(A)6 |
| 3 | 119812307 | 119812318 | p4.(TTCC)3 |
| 3 | 128199894 | 128199898 | p1.(G)5 |
| 3 | 128200024 | 128200028 | p1.(G)5 |
| 3 | 128204623 | 128204628 | p1.(C)6 |
| 3 | 128204842 | 128204847 | p1.(C)6 |
| 3 | 128204873 | 128204877 | p1.(C)5 |
| 3 | 128204992 | 128204996 | p1.(C)5 |
| 3 | 128205004 | 128205008 | p1.(C)5 |
| 3 | 128205139 | 128205143 | p1.(C)5 |
| 3 | 128205878 | 128205889 | p3.(CGG)4 |
| 3 | 128339230 | 128339234 | p1.(C)5 |
| 3 | 128344748 | 128344752 | p1.(T)5 |
| 3 | 128351003 | 128351014 | p4.(GGAA)3 |
| 3 | 128889268 | 128889272 | p1.(A)5 |
| 3 | 128889416 | 128889421 | p1.(A)6 |
| 3 | 128889948 | 128889952 | p1.(T)5 |
| 3 | 134670328 | 134670333 | p1.(G)6 |
| 3 | 134670484 | 134670488 | p1.(C)5 |
| 3 | 134670543 | 134670547 | p1.(G)5 |
| 3 | 134670605 | 134670609 | p1.(T)5 |
| 3 | 134670669 | 134670673 | p1.(A)5 |
| 3 | 134851675 | 134851679 | p1.(A)5 |
| 3 | 134851793 | 134851797 | p1.(C)5 |
| 3 | 134851848 | 134851853 | p1.(C)6 |
| 3 | 134872994 | 134872998 | p1.(C)5 |
| 3 | 134885771 | 134885775 | p1.(T)5 |
| 3 | 134967191 | 134967195 | p1.(C)5 |
| 3 | 134978012 | 134978016 | p1.(G)5 |
| 3 | 138664567 | 138664571 | p1.(G)5 |
| 3 | 138664694 | 138664698 | p1.(G)5 |
| 3 | 138664711 | 138664715 | p1.(G)5 |
| 3 | 138664761 | 138664766 | p1.(G)6 |
| 3 | 138664989 | 138664994 | p1.(G)6 |
| 3 | 138665121 | 138665132 | p3.(GCG)4 |
| 3 | 138665398 | 138665402 | p1.(G)5 |
| 3 | 138665446 | 138665450 | p1.(C)5 |
| 3 | 138665525 | 138665529 | p1.(C)5 |
| 3 | 142168447 | 142168451 | p1.(A)5 |
| 3 | 142176505 | 142176509 | p1.(A)5 |
| 3 | 142178110 | 142178114 | p1.(A)5 |
| 3 | 142178158 | 142178162 | p1.(T)5 |
| 3 | 142180938 | 142180943 | p1.(A)6 |
| 3 | 142185320 | 142185324 | p1.(T)5 |
| 3 | 142185379 | 142185384 | p1.(A)6 |
| 3 | 142186845 | 142186850 | p1.(T)6 |
| 3 | 142186918 | 142186923 | p1.(A)6 |
| 3 | 142188260 | 142188264 | p1.(A)5 |
| 3 | 142188304 | 142188308 | p1.(A)5 |
| 3 | 142188315 | 142188319 | p1.(A)5 |
| 3 | 142188414 | 142188418 | p1.(G)5 |
| 3 | 142188929 | 142188933 | p1.(T)5 |
| 3 | 142204082 | 142204086 | p1.(A)5 |
| 3 | 142204129 | 142204134 | p1.(A)6 |
| 3 | 142211980 | 142211985 | p1.(T)6 |
| 3 | 142212118 | 142212122 | p1.(T)5 |
| 3 | 142212161 | 142212166 | p1.(A)6 |
| 3 | 142217539 | 142217543 | p1.(A)5 |
| 3 | 142217557 | 142217563 | p1.(T)7 |
| 3 | 142217620 | 142217624 | p1.(A)5 |
| 3 | 142226777 | 142226781 | p1.(A)5 |
| 3 | 142242900 | 142242904 | p1.(C)5 |
| 3 | 142243036 | 142243040 | p1.(T)5 |
| 3 | 142253926 | 142253930 | p1.(T)5 |
| 3 | 142254937 | 142254941 | p1.(A)5 |
| 3 | 142254981 | 142254986 | p1.(T)6 |
| 3 | 142255005 | 142255010 | p1.(A)6 |
| 3 | 142266651 | 142266655 | p1.(A)5 |
| 3 | 142269140 | 142269144 | p1.(A)5 |
| 3 | 142269152 | 142269156 | p1.(A)5 |
| 3 | 142272097 | 142272102 | p1.(A)6 |
| 3 | 142272249 | 142272253 | p1.(A)5 |
| 3 | 142272577 | 142272581 | p1.(A)5 |
| 3 | 142272654 | 142272658 | p1.(A)5 |
| 3 | 142274740 | 142274749 | p1.(T)10 |
| 3 | 142274988 | 142274995 | p1.(A)8 |
| 3 | 142275275 | 142275280 | p1.(A)6 |
| 3 | 142275421 | 142275426 | p1.(A)6 |
| 3 | 142278207 | 142278211 | p1.(A)5 |
| 3 | 142281061 | 142281065 | p1.(A)5 |
| 3 | 142281272 | 142281276 | p1.(T)5 |
| 3 | 142281423 | 142281427 | p1.(A)5 |
| 3 | 142281435 | 142281440 | p1.(A)6 |
| 3 | 142281476 | 142281480 | p1.(A)5 |
| 3 | 142281489 | 142281493 | p1.(A)5 |
| 3 | 142281514 | 142281518 | p1.(T)5 |
| 3 | 142281578 | 142281583 | p1.(A)6 |
| 3 | 142281708 | 142281712 | p1.(A)5 |
| 3 | 142281785 | 142281789 | p1.(A)5 |
| 3 | 142281818 | 142281822 | p1.(A)5 |
| 3 | 142281842 | 142281846 | p1.(A)5 |
| 3 | 142297540 | 142297544 | p1.(C)5 |
| 3 | 149238651 | 149238655 | p1.(G)5 |
| 3 | 149260194 | 149260211 | p3.(CTG)6 |
| 3 | 149260290 | 149260294 | p1.(G)5 |
| 3 | 149290774 | 149290779 | p1.(T)6 |
| 3 | 149374711 | 149374715 | p1.(G)5 |
| 3 | 149374852 | 149374856 | p1.(C)5 |
| 3 | 149375071 | 149375075 | p1.(G)5 |
| 3 | 149375101 | 149375105 | p1.(A)5 |
| 3 | 155588695 | 155588699 | p1.(G)5 |
| 3 | 155621685 | 155621691 | p1.(A)7 |
| 3 | 155621758 | 155621762 | p1.(T)5 |
| 3 | 155628498 | 155628502 | p1.(A)5 |
| 3 | 155629044 | 155629048 | p1.(A)5 |
| 3 | 155632332 | 155632336 | p1.(A)5 |
| 3 | 155637130 | 155637134 | p1.(A)5 |
| 3 | 155640070 | 155640074 | p1.(T)5 |
| 3 | 155640086 | 155640090 | p1.(A)5 |
| 3 | 155643020 | 155643024 | p1.(T)5 |
| 3 | 155649541 | 155649545 | p1.(T)5 |
| 3 | 155649624 | 155649628 | p1.(T)5 |
| 3 | 158289044 | 158289048 | p1.(T)5 |
| 3 | 158310271 | 158310275 | p1.(T)5 |
| 3 | 158310379 | 158310386 | p1.(T)8 |
| 3 | 158315957 | 158315961 | p1.(T)5 |
| 3 | 158322940 | 158322944 | p1.(G)5 |
| 3 | 158322983 | 158322988 | p1.(A)6 |
| 3 | 168802839 | 168802843 | p1.(A)5 |
| 3 | 168807891 | 168807895 | p1.(T)5 |
| 3 | 168813037 | 168813041 | p1.(A)5 |
| 3 | 168833257 | 168833268 | p1.(T)7(C)5 |
| 3 | 168833279 | 168833284 | p1.(T)6 |
| 3 | 168833437 | 168833441 | p1.(G)5 |
| 3 | 168833560 | 168833565 | p1.(A)6 |
| 3 | 168833605 | 168833610 | p1.(T)6 |
| 3 | 168833647 | 168833651 | p1.(T)5 |
| 3 | 168834343 | 168834348 | p1.(A)6 |
| 3 | 168845646 | 168845650 | p1.(A)5 |
| 3 | 168845715 | 168845719 | p1.(T)5 |
| 3 | 176750928 | 176750932 | p1.(A)5 |
| 3 | 176755963 | 176755967 | p1.(A)5 |
| 3 | 176768307 | 176768311 | p1.(A)5 |
| 3 | 176769342 | 176769347 | p1.(T)6 |
| 3 | 176771709 | 176771713 | p1.(A)5 |
| 3 | 178916662 | 178916666 | p1.(C)5 |
| 3 | 178916781 | 178916785 | p1.(C)5 |
| 3 | 178916857 | 178916862 | p1.(T)6 |
| 3 | 178916894 | 178916898 | p1.(T)5 |
| 3 | 178916905 | 178916909 | p1.(T)5 |
| 3 | 178919194 | 178919200 | p1.(A)7 |
| 3 | 178919303 | 178919307 | p1.(A)5 |
| 3 | 178921526 | 178921530 | p1.(A)5 |
| 3 | 178922278 | 178922283 | p1.(T)6 |
| 3 | 178927965 | 178927970 | p1.(T)6 |
| 3 | 178928039 | 178928043 | p1.(A)5 |
| 3 | 178928133 | 178928137 | p1.(T)5 |
| 3 | 178937471 | 178937475 | p1.(A)5 |
| 3 | 178941854 | 178941862 | p1.(T)9 |
| 3 | 178941881 | 178941885 | p1.(T)5 |
| 3 | 178942518 | 178942522 | p1.(A)5 |
| 3 | 178942597 | 178942601 | p1.(A)5 |
| 3 | 178948003 | 178948007 | p1.(T)5 |
| 3 | 178948037 | 178948041 | p1.(T)5 |
| 3 | 178948055 | 178948060 | p1.(A)6 |
| 3 | 178952104 | 178952108 | p1.(A)5 |
| 3 | 181430202 | 181430207 | p1.(G)6 |
| 3 | 181430206 | 181430217 | p3.(GGC)4 |
| 3 | 181430251 | 181430255 | p1.(A)5 |
| 3 | 181430546 | 181430550 | p1.(C)5 |
| 3 | 181430901 | 181430906 | p1.(C)6 |
| 3 | 181431011 | 181431015 | p1.(C)5 |
| 3 | 183209685 | 183209691 | p1.(G)7 |
| 3 | 183210422 | 183210426 | p1.(C)5 |
| 3 | 183217395 | 183217399 | p1.(T)5 |
| 3 | 183217498 | 183217502 | p1.(G)5 |
| 3 | 185774991 | 185774995 | p1.(G)5 |
| 3 | 185775264 | 185775268 | p1.(T)5 |
| 3 | 185775275 | 185775279 | p1.(A)5 |
| 3 | 185782229 | 185782233 | p1.(C)5 |
| 3 | 185783667 | 185783671 | p1.(G)5 |
| 3 | 185783736 | 185783740 | p1.(G)5 |
| 3 | 185783742 | 185783746 | p1.(G)5 |
| 3 | 185797673 | 185797677 | p1.(G)5 |
| 3 | 185797721 | 185797726 | p1.(G)6 |
| 3 | 185797728 | 185797732 | p1.(G)5 |
| 3 | 185797801 | 185797805 | p1.(G)5 |
| 3 | 185797835 | 185797840 | p1.(G)6 |
| 3 | 185823443 | 185823447 | p1.(A)5 |
| 3 | 185823714 | 185823718 | p1.(G)5 |
| 3 | 186501339 | 186501344 | p1.(A)6 |
| 3 | 186501389 | 186501393 | p1.(T)5 |
| 3 | 186502742 | 186502748 | p1.(T)7 |
| 3 | 186502865 | 186502869 | p1.(C)5 |
| 3 | 186503760 | 186503764 | p1.(A)5 |
| 3 | 186504283 | 186504287 | p1.(T)5 |
| 3 | 186504339 | 186504344 | p1.(A)6 |
| 3 | 186504375 | 186504379 | p1.(A)5 |
| 3 | 186504977 | 186504981 | p1.(T)5 |
| 3 | 186506924 | 186506928 | p1.(G)5 |
| 3 | 187446270 | 187446275 | p1.(G)6 |
| 3 | 187447190 | 187447194 | p1.(G)5 |
| 3 | 187447236 | 187447241 | p1.(G)6 |
| 3 | 187447317 | 187447321 | p1.(G)5 |
| 3 | 187447335 | 187447339 | p1.(G)5 |
| 3 | 187447440 | 187447444 | p1.(G)5 |
| 3 | 187447647 | 187447651 | p1.(G)5 |
| 3 | 187451496 | 187451501 | p1.(A)6 |
| 3 | 188123887 | 188123891 | p1.(T)5 |
| 3 | 188124035 | 188124039 | p1.(A)5 |
| 3 | 188202394 | 188202398 | p1.(C)5 |
| 3 | 188242550 | 188242554 | p1.(C)5 |
| 3 | 188326942 | 188326946 | p1.(T)5 |
| 3 | 188327027 | 188327031 | p1.(C)5 |
| 3 | 188327199 | 188327213 | p5.(CAGCC)3 |
| 3 | 188327388 | 188327392 | p1.(C)5 |
| 3 | 188327441 | 188327445 | p1.(G)5 |
| 3 | 188327604 | 188327608 | p1.(C)5 |
| 3 | 188426133 | 188426137 | p1.(A)5 |
| 3 | 188477889 | 188477896 | p1.(T)8 |
| 3 | 188592312 | 188592316 | p1.(A)5 |
| 3 | 195785160 | 195785164 | p1.(A)5 |
| 3 | 195792356 | 195792360 | p1.(T)5 |
| 3 | 195794379 | 195794383 | p1.(A)5 |
| 3 | 195798301 | 195798305 | p1.(C)5 |
| 3 | 195798375 | 195798379 | p1.(A)5 |
| 3 | 195798975 | 195798979 | p1.(T)5 |
| 3 | 195800949 | 195800953 | p1.(T)5 |
| 3 | 195802038 | 195802042 | p1.(A)5 |
| 3 | 195802087 | 195802091 | p1.(T)5 |
| 4 | 1795648 | 1795652 | p1.(C)5 |
| 4 | 1795654 | 1795658 | p1.(C)5 |
| 4 | 1801055 | 1801059 | p1.(C)5 |
| 4 | 1801064 | 1801068 | p1.(G)5 |
| 4 | 1803553 | 1803557 | p1.(C)5 |
| 4 | 1806181 | 1806187 | p1.(C)7 |
| 4 | 1806204 | 1806208 | p1.(C)5 |
| 4 | 1807211 | 1807215 | p1.(G)5 |
| 4 | 1807357 | 1807361 | p1.(A)5 |
| 4 | 1807545 | 1807549 | p1.(C)5 |
| 4 | 1808312 | 1808317 | p1.(G)6 |
| 4 | 1808898 | 1808902 | p1.(C)5 |
| 4 | 1808951 | 1808955 | p1.(C)5 |
| 4 | 1808972 | 1808976 | p1.(G)5 |
| 4 | 1809111 | 1809126 | p2.(TG)8 |
| 4 | 1809128 | 1809145 | p2.(GT)9 |
| 4 | 1809269 | 1809274 | p1.(G)6 |
| 4 | 1809312 | 1809316 | p1.(C)5 |
| 4 | 1902558 | 1902562 | p1.(G)5 |
| 4 | 1902712 | 1902716 | p1.(C)5 |
| 4 | 1902733 | 1902737 | p1.(A)5 |
| 4 | 1902857 | 1902861 | p1.(A)5 |
| 4 | 1902973 | 1902977 | p1.(A)5 |
| 4 | 1918589 | 1918593 | p1.(A)5 |
| 4 | 1918603 | 1918607 | p1.(A)5 |
| 4 | 1918709 | 1918713 | p1.(A)5 |
| 4 | 1919853 | 1919862 | p1.(T)10 |
| 4 | 1919987 | 1919991 | p1.(G)5 |
| 4 | 1920318 | 1920322 | p1.(T)5 |
| 4 | 1920334 | 1920338 | p1.(A)5 |
| 4 | 1936885 | 1936891 | p1.(A)7 |
| 4 | 1941386 | 1941390 | p1.(A)5 |
| 4 | 1941510 | 1941521 | p3.(AAT)4 |
| 4 | 1952883 | 1952887 | p1.(A)5 |
| 4 | 1952910 | 1952914 | p1.(A)5 |
| 4 | 1953826 | 1953830 | p1.(C)5 |
| 4 | 1955041 | 1955045 | p1.(T)5 |
| 4 | 1955146 | 1955151 | p1.(A)6 |
| 4 | 1955190 | 1955194 | p1.(C)5 |
| 4 | 1957419 | 1957424 | p1.(G)6 |
| 4 | 1957738 | 1957742 | p1.(A)5 |
| 4 | 1957747 | 1957751 | p1.(C)5 |
| 4 | 1957795 | 1957799 | p1.(T)5 |
| 4 | 1957857 | 1957861 | p1.(G)5 |
| 4 | 1957881 | 1957885 | p1.(G)5 |
| 4 | 1957909 | 1957913 | p1.(A)5 |
| 4 | 1959653 | 1959657 | p1.(T)5 |
| 4 | 1959740 | 1959744 | p1.(C)5 |
| 4 | 1977066 | 1977071 | p1.(A)6 |
| 4 | 1978235 | 1978239 | p1.(A)5 |
| 4 | 1980559 | 1980566 | p1.(C)8 |
| 4 | 25665813 | 25665818 | p1.(C)6 |
| 4 | 25665901 | 25665905 | p1.(T)5 |
| 4 | 25667740 | 25667744 | p1.(T)5 |
| 4 | 25667751 | 25667755 | p1.(A)5 |
| 4 | 25669536 | 25669540 | p1.(G)5 |
| 4 | 25672366 | 25672371 | p1.(A)6 |
| 4 | 25672400 | 25672404 | p1.(A)5 |
| 4 | 25672408 | 25672412 | p1.(A)5 |
| 4 | 25676023 | 25676028 | p1.(C)6 |
| 4 | 25676120 | 25676124 | p1.(C)5 |
| 4 | 25676185 | 25676196 | p3.(CAC)4 |
| 4 | 25677772 | 25677776 | p1.(T)5 |
| 4 | 25678148 | 25678162 | p3.(GCT)5 |
| 4 | 25678396 | 25678401 | p1.(G)6 |
| 4 | 41747792 | 41747812 | p3.(CGC)7 |
| 4 | 41747903 | 41747907 | p1.(C)5 |
| 4 | 41747966 | 41747970 | p1.(C)5 |
| 4 | 41748008 | 41748019 | p3.(GCC)4 |
| 4 | 41748077 | 41748081 | p1.(C)5 |
| 4 | 41748093 | 41748097 | p1.(C)5 |
| 4 | 41748119 | 41748130 | p3.(CCG)4 |
| 4 | 41748151 | 41748155 | p1.(G)5 |
| 4 | 41748179 | 41748183 | p1.(C)5 |
| 4 | 41748245 | 41748249 | p1.(T)5 |
| 4 | 54243963 | 54243967 | p1.(G)5 |
| 4 | 54250052 | 54250057 | p1.(T)6 |
| 4 | 54255957 | 54255968 | p4.(TTTG)3 |
| 4 | 54255969 | 54255973 | p1.(T)5 |
| 4 | 54256016 | 54256020 | p1.(G)5 |
| 4 | 54265884 | 54265893 | p1.(T)10 |
| 4 | 54294251 | 54294255 | p1.(T)5 |
| 4 | 54319075 | 54319080 | p1.(T)6 |
| 4 | 54319188 | 54319197 | p2.(AG)5 |
| 4 | 54319248 | 54319261 | p2.(AG)7 |
| 4 | 54324888 | 54324892 | p1.(A)5 |
| 4 | 55127451 | 55127455 | p1.(T)5 |
| 4 | 55129961 | 55129965 | p1.(G)5 |
| 4 | 55131076 | 55131081 | p1.(T)6 |
| 4 | 55131167 | 55131171 | p1.(T)5 |
| 4 | 55133838 | 55133842 | p1.(A)5 |
| 4 | 55136788 | 55136794 | p1.(T)7 |
| 4 | 55138608 | 55138612 | p1.(G)5 |
| 4 | 55139691 | 55139695 | p1.(T)5 |
| 4 | 55144081 | 55144085 | p1.(A)5 |
| 4 | 55146474 | 55146478 | p1.(T)5 |
| 4 | 55151549 | 55151553 | p1.(A)5 |
| 4 | 55151636 | 55151640 | p1.(T)5 |
| 4 | 55151647 | 55151652 | p1.(A)6 |
| 4 | 55152055 | 55152059 | p1.(A)5 |
| 4 | 55155276 | 55155280 | p1.(A)5 |
| 4 | 55156487 | 55156492 | p1.(A)6 |
| 4 | 55156579 | 55156583 | p1.(A)5 |
| 4 | 55156730 | 55156734 | p1.(G)5 |
| 4 | 55561665 | 55561669 | p1.(T)5 |
| 4 | 55561719 | 55561730 | p4.(CCAT)3 |
| 4 | 55564705 | 55564709 | p1.(A)5 |
| 4 | 55589841 | 55589845 | p1.(T)5 |
| 4 | 55595550 | 55595555 | p1.(T)6 |
| 4 | 55602749 | 55602753 | p1.(T)5 |
| 4 | 55603380 | 55603384 | p1.(A)5 |
| 4 | 55946088 | 55946092 | p1.(G)5 |
| 4 | 55948210 | 55948214 | p1.(T)5 |
| 4 | 55948753 | 55948757 | p1.(T)5 |
| 4 | 55953819 | 55953830 | p3.(TCC)4 |
| 4 | 55955928 | 55955932 | p1.(A)5 |
| 4 | 55961127 | 55961132 | p1.(A)6 |
| 4 | 55964855 | 55964861 | p1.(T)7 |
| 4 | 55968075 | 55968079 | p1.(A)5 |
| 4 | 55968579 | 55968583 | p1.(G)5 |
| 4 | 55971148 | 55971152 | p1.(C)5 |
| 4 | 55972858 | 55972863 | p1.(T)6 |
| 4 | 55972885 | 55972889 | p1.(T)5 |
| 4 | 55973958 | 55973962 | p1.(G)5 |
| 4 | 55976725 | 55976729 | p1.(T)5 |
| 4 | 55976739 | 55976746 | p1.(A)8 |
| 4 | 55976833 | 55976837 | p1.(G)5 |
| 4 | 55976876 | 55976880 | p1.(C)5 |
| 4 | 55976931 | 55976935 | p1.(T)5 |
| 4 | 55979581 | 55979585 | p1.(A)5 |
| 4 | 55979612 | 55979616 | p1.(T)5 |
| 4 | 55979652 | 55979658 | p1.(A)7 |
| 4 | 55981089 | 55981093 | p1.(T)5 |
| 4 | 55981509 | 55981513 | p1.(T)5 |
| 4 | 55984971 | 55984975 | p1.(A)5 |
| 4 | 55987311 | 55987315 | p1.(T)5 |
| 4 | 66197678 | 66197682 | p1.(A)5 |
| 4 | 66213912 | 66213916 | p1.(T)5 |
| 4 | 66218764 | 66218768 | p1.(A)5 |
| 4 | 66230873 | 66230877 | p1.(T)5 |
| 4 | 66286238 | 66286243 | p1.(T)6 |
| 4 | 66286255 | 66286260 | p1.(T)6 |
| 4 | 66356229 | 66356233 | p1.(T)5 |
| 4 | 66356423 | 66356428 | p1.(G)6 |
| 4 | 66467373 | 66467377 | p1.(T)5 |
| 4 | 66467573 | 66467578 | p1.(T)6 |
| 4 | 66467646 | 66467650 | p1.(T)5 |
| 4 | 66535395 | 66535399 | p1.(G)5 |
| 4 | 66535403 | 66535414 | p3.(CGC)4 |
| 4 | 66535417 | 66535421 | p1.(G)5 |
| 4 | 66535558 | 66535563 | p1.(C)6 |
| 4 | 87967419 | 87967423 | p1.(T)5 |
| 4 | 87967836 | 87967843 | p1.(T)8 |
| 4 | 87968333 | 87968337 | p1.(C)5 |
| 4 | 87968534 | 87968545 | p4.(CCTC)3 |
| 4 | 87968551 | 87968555 | p1.(A)5 |
| 4 | 87968676 | 87968681 | p1.(A)6 |
| 4 | 88016058 | 88016062 | p1.(C)5 |
| 4 | 88016086 | 88016090 | p1.(A)5 |
| 4 | 88035512 | 88035516 | p1.(T)5 |
| 4 | 88035572 | 88035583 | p4.(CAGC)3 |
| 4 | 88035615 | 88035619 | p1.(C)5 |
| 4 | 88035693 | 88035697 | p1.(C)5 |
| 4 | 88035715 | 88035720 | p1.(C)6 |
| 4 | 88035736 | 88035741 | p1.(C)6 |
| 4 | 88035786 | 88035790 | p1.(C)5 |
| 4 | 88035822 | 88035827 | p1.(A)6 |
| 4 | 88035872 | 88035876 | p1.(G)5 |
| 4 | 88035996 | 88036001 | p1.(C)6 |
| 4 | 88036039 | 88036043 | p1.(C)5 |
| 4 | 88036275 | 88036279 | p1.(C)5 |
| 4 | 88036440 | 88036444 | p1.(A)5 |
| 4 | 88047293 | 88047298 | p1.(C)6 |
| 4 | 88048164 | 88048173 | p1.(T)10 |
| 4 | 88048787 | 88048791 | p1.(T)5 |
| 4 | 88048825 | 88048830 | p1.(A)6 |
| 4 | 88053466 | 88053471 | p1.(A)6 |
| 4 | 88056720 | 88056727 | p1.(T)8 |
| 4 | 88056889 | 88056893 | p1.(T)5 |
| 4 | 88056910 | 88056914 | p1.(A)5 |
| 4 | 99264305 | 99264310 | p1.(A)6 |
| 4 | 99264406 | 99264410 | p1.(A)5 |
| 4 | 99313094 | 99313098 | p1.(T)5 |
| 4 | 99337959 | 99337963 | p1.(A)5 |
| 4 | 99341159 | 99341163 | p1.(T)5 |
| 4 | 99342494 | 99342498 | p1.(G)5 |
| 4 | 106155594 | 106155598 | p1.(T)5 |
| 4 | 106155700 | 106155704 | p1.(A)5 |
| 4 | 106155779 | 106155784 | p1.(A)6 |
| 4 | 106156073 | 106156077 | p1.(A)5 |
| 4 | 106156082 | 106156086 | p1.(T)5 |
| 4 | 106156289 | 106156300 | p3.(CAC)4 |
| 4 | 106156321 | 106156325 | p1.(C)5 |
| 4 | 106156365 | 106156369 | p1.(A)5 |
| 4 | 106156652 | 106156656 | p1.(T)5 |
| 4 | 106156759 | 106156763 | p1.(C)5 |
| 4 | 106156936 | 106156941 | p1.(G)6 |
| 4 | 106157060 | 106157064 | p1.(A)5 |
| 4 | 106157174 | 106157178 | p1.(A)5 |
| 4 | 106157336 | 106157340 | p1.(A)5 |
| 4 | 106157385 | 106157389 | p1.(C)5 |
| 4 | 106157657 | 106157661 | p1.(T)5 |
| 4 | 106157797 | 106157801 | p1.(A)5 |
| 4 | 106157879 | 106157883 | p1.(T)5 |
| 4 | 106158109 | 106158113 | p1.(A)5 |
| 4 | 106158447 | 106158452 | p1.(A)6 |
| 4 | 106162508 | 106162512 | p1.(A)5 |
| 4 | 106162524 | 106162528 | p1.(T)5 |
| 4 | 106180768 | 106180772 | p1.(T)5 |
| 4 | 106190759 | 106190763 | p1.(T)5 |
| 4 | 106193850 | 106193855 | p1.(A)6 |
| 4 | 106194000 | 106194004 | p1.(A)5 |
| 4 | 106196282 | 106196293 | p3.(CAG)4 |
| 4 | 106196300 | 106196311 | p3.(CAG)4 |
| 4 | 106196921 | 106196925 | p1.(A)5 |
| 4 | 106197245 | 106197249 | p1.(G)5 |
| 4 | 106197435 | 106197439 | p1.(A)5 |
| 4 | 106197506 | 106197511 | p1.(A)6 |
| 4 | 106197682 | 106197686 | p1.(C)5 |
| 4 | 123374926 | 123374930 | p1.(T)5 |
| 4 | 153244148 | 153244152 | p1.(C)5 |
| 4 | 153244156 | 153244161 | p1.(C)6 |
| 4 | 153244308 | 153244312 | p1.(A)5 |
| 4 | 153249361 | 153249366 | p1.(T)6 |
| 4 | 153249546 | 153249550 | p1.(A)5 |
| 4 | 153253877 | 153253883 | p1.(A)7 |
| 4 | 153268090 | 153268094 | p1.(T)5 |
| 4 | 153268228 | 153268241 | p1.(A)14 |
| 4 | 153332463 | 153332467 | p1.(T)5 |
| 4 | 153332605 | 153332619 | p3.(CTC)5 |
| 5 | 1254606 | 1254610 | p1.(C)5 |
| 5 | 1254623 | 1254627 | p1.(G)5 |
| 5 | 1255464 | 1255468 | p1.(A)5 |
| 5 | 1278878 | 1278882 | p1.(G)5 |
| 5 | 1282589 | 1282593 | p1.(A)5 |
| 5 | 1293577 | 1293581 | p1.(G)5 |
| 5 | 1293665 | 1293669 | p1.(G)5 |
| 5 | 1293687 | 1293691 | p1.(G)5 |
| 5 | 1294033 | 1294037 | p1.(G)5 |
| 5 | 1294078 | 1294083 | p1.(G)6 |
| 5 | 1294324 | 1294328 | p1.(C)5 |
| 5 | 1294361 | 1294365 | p1.(G)5 |
| 5 | 1294441 | 1294445 | p1.(G)5 |
| 5 | 1294587 | 1294591 | p1.(C)5 |
| 5 | 1294602 | 1294606 | p1.(C)5 |
| 5 | 1294665 | 1294676 | p1.(G)7(C)5 |
| 5 | 1294786 | 1294790 | p1.(G)5 |
| 5 | 35857056 | 35857067 | p2.(CT)6 |
| 5 | 35857111 | 35857116 | p1.(T)6 |
| 5 | 35874591 | 35874596 | p1.(T)6 |
| 5 | 35874637 | 35874643 | p1.(A)7 |
| 5 | 35875697 | 35875701 | p1.(T)5 |
| 5 | 35876072 | 35876076 | p1.(T)5 |
| 5 | 35876454 | 35876458 | p1.(C)5 |
| 5 | 38481718 | 38481723 | p1.(A)6 |
| 5 | 38481785 | 38481789 | p1.(A)5 |
| 5 | 38486086 | 38486090 | p1.(T)5 |
| 5 | 38489248 | 38489252 | p1.(A)5 |
| 5 | 38490360 | 38490365 | p1.(A)6 |
| 5 | 38496537 | 38496541 | p1.(T)5 |
| 5 | 38496667 | 38496671 | p1.(T)5 |
| 5 | 38496703 | 38496707 | p1.(A)5 |
| 5 | 38499634 | 38499639 | p1.(T)6 |
| 5 | 38499688 | 38499692 | p1.(A)5 |
| 5 | 38502761 | 38502766 | p1.(T)6 |
| 5 | 38506008 | 38506012 | p1.(T)5 |
| 5 | 38506172 | 38506176 | p1.(A)5 |
| 5 | 38506742 | 38506749 | p1.(A)8 |
| 5 | 38510573 | 38510577 | p1.(A)5 |
| 5 | 38510609 | 38510613 | p1.(A)5 |
| 5 | 38510729 | 38510733 | p1.(T)5 |
| 5 | 38510792 | 38510796 | p1.(A)5 |
| 5 | 38510829 | 38510833 | p1.(A)5 |
| 5 | 38511940 | 38511944 | p1.(A)5 |
| 5 | 38512069 | 38512073 | p1.(A)5 |
| 5 | 38523590 | 38523594 | p1.(A)5 |
| 5 | 38523689 | 38523693 | p1.(A)5 |
| 5 | 38530610 | 38530614 | p1.(T)5 |
| 5 | 38942487 | 38942492 | p1.(A)6 |
| 5 | 38945177 | 38945183 | p1.(A)7 |
| 5 | 38945715 | 38945719 | p1.(A)5 |
| 5 | 38945793 | 38945798 | p1.(T)6 |
| 5 | 38947421 | 38947425 | p1.(C)5 |
| 5 | 38950626 | 38950631 | p1.(T)6 |
| 5 | 38950702 | 38950706 | p1.(T)5 |
| 5 | 38950768 | 38950772 | p1.(A)5 |
| 5 | 38954869 | 38954873 | p1.(T)5 |
| 5 | 38958824 | 38958828 | p1.(T)5 |
| 5 | 38959898 | 38959902 | p1.(T)5 |
| 5 | 38959950 | 38959954 | p1.(A)5 |
| 5 | 38960610 | 38960614 | p1.(A)5 |
| 5 | 38963152 | 38963156 | p1.(A)5 |
| 5 | 38967334 | 38967339 | p1.(A)6 |
| 5 | 38967467 | 38967471 | p1.(T)5 |
| 5 | 38975712 | 38975716 | p1.(G)5 |
| 5 | 44305137 | 44305141 | p1.(T)5 |
| 5 | 44388711 | 44388715 | p1.(A)5 |
| 5 | 44388715 | 44388732 | p3.(AGC)6 |
| 5 | 55237477 | 55237482 | p1.(C)6 |
| 5 | 55237512 | 55237517 | p1.(T)6 |
| 5 | 55237520 | 55237524 | p1.(T)5 |
| 5 | 55237562 | 55237566 | p1.(T)5 |
| 5 | 55243421 | 55243445 | p1.(A)25 |
| 5 | 55247858 | 55247862 | p1.(T)5 |
| 5 | 55247869 | 55247875 | p1.(T)7 |
| 5 | 55252033 | 55252037 | p1.(T)5 |
| 5 | 55259338 | 55259342 | p1.(A)5 |
| 5 | 55264203 | 55264207 | p1.(T)5 |
| 5 | 55264211 | 55264215 | p1.(T)5 |
| 5 | 55265589 | 55265593 | p1.(T)5 |
| 5 | 55265686 | 55265690 | p1.(A)5 |
| 5 | 56155721 | 56155725 | p1.(A)5 |
| 5 | 56161262 | 56161266 | p1.(A)5 |
| 5 | 56161288 | 56161292 | p1.(T)5 |
| 5 | 56161813 | 56161817 | p1.(T)5 |
| 5 | 56168460 | 56168464 | p1.(T)5 |
| 5 | 56168639 | 56168643 | p1.(T)5 |
| 5 | 56170991 | 56170995 | p1.(G)5 |
| 5 | 56171018 | 56171022 | p1.(G)5 |
| 5 | 56174794 | 56174800 | p1.(T)7 |
| 5 | 56176901 | 56176905 | p1.(T)5 |
| 5 | 56177104 | 56177108 | p1.(T)5 |
| 5 | 56177383 | 56177389 | p1.(T)7 |
| 5 | 56177849 | 56177872 | p3.(CAA)8 |
| 5 | 56178258 | 56178263 | p1.(A)6 |
| 5 | 56178577 | 56178588 | p3.(GAA)4 |
| 5 | 56181758 | 56181762 | p1.(G)5 |
| 5 | 56189345 | 56189349 | p1.(T)5 |
| 5 | 56189555 | 56189562 | p1.(A)8 |
| 5 | 67522546 | 67522550 | p1.(A)5 |
| 5 | 67522700 | 67522705 | p1.(G)6 |
| 5 | 67522741 | 67522747 | p1.(A)7 |
| 5 | 67575567 | 67575572 | p1.(T)6 |
| 5 | 67588943 | 67588947 | p1.(A)5 |
| 5 | 67588972 | 67588976 | p1.(T)5 |
| 5 | 67590411 | 67590415 | p1.(A)5 |
| 5 | 67591240 | 67591244 | p1.(T)5 |
| 5 | 67591259 | 67591263 | p1.(A)5 |
| 5 | 112102013 | 112102017 | p1.(T)5 |
| 5 | 112102932 | 112102936 | p1.(A)5 |
| 5 | 112116587 | 112116591 | p1.(A)5 |
| 5 | 112151185 | 112151189 | p1.(T)5 |
| 5 | 112155032 | 112155036 | p1.(A)5 |
| 5 | 112157585 | 112157589 | p1.(T)5 |
| 5 | 112157696 | 112157700 | p1.(A)5 |
| 5 | 112162793 | 112162797 | p1.(T)5 |
| 5 | 112162804 | 112162808 | p1.(G)5 |
| 5 | 112162950 | 112162954 | p1.(T)5 |
| 5 | 112164604 | 112164608 | p1.(A)5 |
| 5 | 112164664 | 112164668 | p1.(A)5 |
| 5 | 112170747 | 112170751 | p1.(T)5 |
| 5 | 112173394 | 112173398 | p1.(G)5 |
| 5 | 112173561 | 112173565 | p1.(A)5 |
| 5 | 112173690 | 112173694 | p1.(T)5 |
| 5 | 112173831 | 112173835 | p1.(A)5 |
| 5 | 112175101 | 112175105 | p1.(T)5 |
| 5 | 112175651 | 112175655 | p1.(A)5 |
| 5 | 112175676 | 112175685 | p2.(AG)5 |
| 5 | 112175952 | 112175957 | p1.(A)6 |
| 5 | 112175993 | 112176004 | p3.(GAT)4 |
| 5 | 112176064 | 112176069 | p1.(A)6 |
| 5 | 112176193 | 112176197 | p1.(G)5 |
| 5 | 112176345 | 112176349 | p1.(A)5 |
| 5 | 112176400 | 112176404 | p1.(A)5 |
| 5 | 112176521 | 112176525 | p1.(A)5 |
| 5 | 112176575 | 112176579 | p1.(A)5 |
| 5 | 112176596 | 112176600 | p1.(A)5 |
| 5 | 112176661 | 112176666 | p1.(A)6 |
| 5 | 112176676 | 112176681 | p1.(A)6 |
| 5 | 112176740 | 112176744 | p1.(A)5 |
| 5 | 112176856 | 112176860 | p1.(T)5 |
| 5 | 112176892 | 112176903 | p3.(TGA)4 |
| 5 | 112177265 | 112177269 | p1.(C)5 |
| 5 | 112177435 | 112177440 | p1.(A)6 |
| 5 | 112177442 | 112177446 | p1.(A)5 |
| 5 | 112177473 | 112177477 | p1.(A)5 |
| 5 | 112177643 | 112177654 | p3.(GCT)4 |
| 5 | 112177758 | 112177762 | p1.(A)5 |
| 5 | 112177826 | 112177830 | p1.(A)5 |
| 5 | 112177864 | 112177870 | p1.(A)7 |
| 5 | 112178033 | 112178038 | p1.(A)6 |
| 5 | 112178377 | 112178381 | p1.(A)5 |
| 5 | 112178525 | 112178529 | p1.(A)5 |
| 5 | 112179036 | 112179040 | p1.(A)5 |
| 5 | 112179043 | 112179047 | p1.(A)5 |
| 5 | 112179057 | 112179061 | p1.(A)5 |
| 5 | 112179130 | 112179134 | p1.(A)5 |
| 5 | 112179329 | 112179333 | p1.(C)5 |
| 5 | 112179489 | 112179493 | p1.(A)5 |
| 5 | 131289960 | 131289965 | p1.(T)6 |
| 5 | 131290067 | 131290071 | p1.(A)5 |
| 5 | 131296307 | 131296311 | p1.(A)5 |
| 5 | 131298340 | 131298344 | p1.(T)5 |
| 5 | 131310536 | 131310541 | p1.(A)6 |
| 5 | 131312419 | 131312423 | p1.(A)5 |
| 5 | 131321147 | 131321151 | p1.(G)5 |
| 5 | 131323925 | 131323929 | p1.(G)5 |
| 5 | 131325118 | 131325122 | p1.(A)5 |
| 5 | 131329854 | 131329858 | p1.(A)5 |
| 5 | 131347218 | 131347223 | p1.(C)6 |
| 5 | 131347259 | 131347278 | p5.(GGCCC)4 |
| 5 | 131893103 | 131893107 | p1.(C)5 |
| 5 | 131915179 | 131915184 | p1.(T)6 |
| 5 | 131923617 | 131923621 | p1.(T)5 |
| 5 | 131923719 | 131923723 | p1.(A)5 |
| 5 | 131923755 | 131923759 | p1.(A)5 |
| 5 | 131926940 | 131926944 | p1.(A)5 |
| 5 | 131926963 | 131926967 | p1.(A)5 |
| 5 | 131926992 | 131926996 | p1.(A)5 |
| 5 | 131927105 | 131927109 | p1.(T)5 |
| 5 | 131930589 | 131930593 | p1.(A)5 |
| 5 | 131930612 | 131930616 | p1.(A)5 |
| 5 | 131930716 | 131930720 | p1.(A)5 |
| 5 | 131931355 | 131931359 | p1.(T)5 |
| 5 | 131931452 | 131931460 | p1.(A)9 |
| 5 | 131939635 | 131939639 | p1.(A)5 |
| 5 | 131940487 | 131940491 | p1.(T)5 |
| 5 | 131940562 | 131940566 | p1.(A)5 |
| 5 | 131944965 | 131944970 | p1.(T)6 |
| 5 | 131944976 | 131944980 | p1.(A)5 |
| 5 | 131953752 | 131953757 | p1.(T)6 |
| 5 | 131953798 | 131953802 | p1.(A)5 |
| 5 | 131976354 | 131976359 | p1.(T)6 |
| 5 | 131977876 | 131977880 | p1.(A)5 |
| 5 | 131977985 | 131977989 | p1.(A)5 |
| 5 | 131978055 | 131978059 | p1.(A)5 |
| 5 | 132219022 | 132219033 | p4.(ACCT)3 |
| 5 | 132219033 | 132219037 | p1.(T)5 |
| 5 | 132223525 | 132223529 | p1.(T)5 |
| 5 | 132224872 | 132224878 | p1.(A)7 |
| 5 | 132227923 | 132227927 | p1.(T)5 |
| 5 | 132228017 | 132228021 | p1.(T)5 |
| 5 | 132228818 | 132228822 | p1.(A)5 |
| 5 | 132232045 | 132232049 | p1.(T)5 |
| 5 | 132232094 | 132232098 | p1.(T)5 |
| 5 | 132232104 | 132232108 | p1.(C)5 |
| 5 | 132232258 | 132232263 | p1.(A)6 |
| 5 | 132232439 | 132232443 | p1.(T)5 |
| 5 | 132232616 | 132232620 | p1.(T)5 |
| 5 | 132232627 | 132232632 | p1.(T)6 |
| 5 | 132232811 | 132232815 | p1.(T)5 |
| 5 | 132232922 | 132232926 | p1.(G)5 |
| 5 | 132232936 | 132232940 | p1.(A)5 |
| 5 | 132262874 | 132262878 | p1.(G)5 |
| 5 | 132270072 | 132270076 | p1.(A)5 |
| 5 | 132270259 | 132270264 | p1.(T)6 |
| 5 | 132270490 | 132270494 | p1.(T)5 |
| 5 | 132270638 | 132270644 | p1.(A)7 |
| 5 | 132270641 | 132270660 | p5.(AAAAT)4 |
| 5 | 138118985 | 138118989 | p1.(G)5 |
| 5 | 138160447 | 138160458 | p3.(GGA)4 |
| 5 | 138253498 | 138253502 | p1.(T)5 |
| 5 | 138253516 | 138253520 | p1.(A)5 |
| 5 | 138266190 | 138266194 | p1.(A)5 |
| 5 | 138266535 | 138266539 | p1.(A)5 |
| 5 | 138269653 | 138269657 | p1.(A)5 |
| 5 | 142150301 | 142150306 | p1.(C)6 |
| 5 | 142252952 | 142252956 | p1.(T)5 |
| 5 | 142264851 | 142264857 | p1.(T)7 |
| 5 | 142264869 | 142264873 | p1.(A)5 |
| 5 | 142264915 | 142264919 | p1.(A)5 |
| 5 | 142264938 | 142264942 | p1.(A)5 |
| 5 | 142283153 | 142283157 | p1.(A)5 |
| 5 | 142435659 | 142435664 | p1.(T)6 |
| 5 | 142437269 | 142437273 | p1.(A)5 |
| 5 | 142513610 | 142513614 | p1.(C)5 |
| 5 | 142526787 | 142526793 | p1.(C)7 |
| 5 | 142586763 | 142586768 | p1.(C)6 |
| 5 | 142586864 | 142586868 | p1.(C)5 |
| 5 | 142586999 | 142587003 | p1.(T)5 |
| 5 | 149433732 | 149433743 | p3.(CTG)4 |
| 5 | 149441304 | 149441308 | p1.(G)5 |
| 5 | 149447806 | 149447820 | p3.(AGC)5 |
| 5 | 149447875 | 149447879 | p1.(G)5 |
| 5 | 149449788 | 149449792 | p1.(G)5 |
| 5 | 149449859 | 149449864 | p1.(G)6 |
| 5 | 149452997 | 149453001 | p1.(C)5 |
| 5 | 149456951 | 149456955 | p1.(T)5 |
| 5 | 149457796 | 149457800 | p1.(G)5 |
| 5 | 149457820 | 149457824 | p1.(G)5 |
| 5 | 149460366 | 149460370 | p1.(G)5 |
| 5 | 149460476 | 149460481 | p1.(G)6 |
| 5 | 149495286 | 149495292 | p1.(G)7 |
| 5 | 149495322 | 149495326 | p1.(C)5 |
| 5 | 149495457 | 149495461 | p1.(G)5 |
| 5 | 149497192 | 149497196 | p1.(G)5 |
| 5 | 149497245 | 149497249 | p1.(G)5 |
| 5 | 149498311 | 149498315 | p1.(T)5 |
| 5 | 149498364 | 149498369 | p1.(G)6 |
| 5 | 149499679 | 149499683 | p1.(A)5 |
| 5 | 149501606 | 149501610 | p1.(G)5 |
| 5 | 149502612 | 149502616 | p1.(G)5 |
| 5 | 149503828 | 149503832 | p1.(C)5 |
| 5 | 149513452 | 149513456 | p1.(G)5 |
| 5 | 149514303 | 149514307 | p1.(G)5 |
| 5 | 149515273 | 149515277 | p1.(G)5 |
| 5 | 149515369 | 149515373 | p1.(G)5 |
| 5 | 149786443 | 149786447 | p1.(C)5 |
| 5 | 149792325 | 149792330 | p1.(C)6 |
| 5 | 156638281 | 156638290 | p2.(TG)5 |
| 5 | 156655341 | 156655345 | p1.(A)5 |
| 5 | 156670614 | 156670627 | p2.(CT)7 |
| 5 | 156671260 | 156671264 | p1.(T)5 |
| 5 | 156671325 | 156671329 | p1.(C)5 |
| 5 | 156671397 | 156671401 | p1.(T)5 |
| 5 | 158139169 | 158139173 | p1.(G)5 |
| 5 | 158139202 | 158139206 | p1.(G)5 |
| 5 | 158267101 | 158267105 | p1.(A)5 |
| 5 | 158523982 | 158523986 | p1.(T)5 |
| 5 | 158526518 | 158526522 | p1.(C)5 |
| 5 | 158526526 | 158526532 | p1.(A)7 |
| 5 | 158526535 | 158526549 | p1.(A)15 |
| 5 | 170305084 | 170305092 | p1.(T)9 |
| 5 | 170337964 | 170337969 | p1.(T)6 |
| 5 | 170338144 | 170338149 | p1.(A)6 |
| 5 | 170341158 | 170341165 | p1.(T)8 |
| 5 | 170343461 | 170343466 | p1.(T)6 |
| 5 | 170343564 | 170343568 | p1.(A)5 |
| 5 | 170345756 | 170345760 | p1.(T)5 |
| 5 | 170345870 | 170345874 | p1.(A)5 |
| 5 | 170346624 | 170346628 | p1.(A)5 |
| 5 | 170351501 | 170351505 | p1.(A)5 |
| 5 | 170736336 | 170736350 | p5.(GCCCA)3 |
| 5 | 170736508 | 170736518 | p1.(C)6(G)5 |
| 5 | 170738381 | 170738385 | p1.(C)5 |
| 5 | 170814982 | 170814986 | p1.(C)5 |
| 5 | 170818291 | 170818300 | p1.(T)10 |
| 5 | 170818719 | 170818723 | p1.(G)5 |
| 5 | 170818812 | 170818816 | p1.(T)5 |
| 5 | 170819918 | 170819923 | p1.(A)6 |
| 5 | 170827864 | 170827868 | p1.(A)5 |
| 5 | 176516627 | 176516631 | p1.(G)5 |
| 5 | 176517816 | 176517820 | p1.(C)5 |
| 5 | 176517955 | 176517959 | p1.(C)5 |
| 5 | 176519635 | 176519639 | p1.(C)5 |
| 5 | 176520346 | 176520350 | p1.(C)5 |
| 5 | 176520541 | 176520545 | p1.(C)5 |
| 5 | 176522601 | 176522606 | p1.(C)6 |
| 5 | 176523645 | 176523649 | p1.(G)5 |
| 5 | 176523720 | 176523724 | p1.(C)5 |
| 5 | 176523731 | 176523736 | p1.(C)6 |
| 5 | 176524562 | 176524566 | p1.(C)5 |
| 5 | 176524623 | 176524627 | p1.(C)5 |
| 5 | 176562218 | 176562222 | p1.(T)5 |
| 5 | 176562406 | 176562411 | p1.(A)6 |
| 5 | 176562813 | 176562817 | p1.(A)5 |
| 5 | 176562850 | 176562854 | p1.(A)5 |
| 5 | 176562871 | 176562875 | p1.(C)5 |
| 5 | 176563040 | 176563044 | p1.(T)5 |
| 5 | 176618897 | 176618901 | p1.(A)5 |
| 5 | 176619010 | 176619014 | p1.(A)5 |
| 5 | 176631274 | 176631278 | p1.(A)5 |
| 5 | 176636918 | 176636922 | p1.(A)5 |
| 5 | 176636934 | 176636938 | p1.(A)5 |
| 5 | 176637122 | 176637127 | p1.(A)6 |
| 5 | 176637438 | 176637442 | p1.(A)5 |
| 5 | 176637807 | 176637811 | p1.(C)5 |
| 5 | 176638299 | 176638303 | p1.(A)5 |
| 5 | 176638344 | 176638348 | p1.(G)5 |
| 5 | 176638499 | 176638503 | p1.(T)5 |
| 5 | 176638712 | 176638716 | p1.(T)5 |
| 5 | 176638771 | 176638775 | p1.(A)5 |
| 5 | 176638869 | 176638873 | p1.(A)5 |
| 5 | 176665224 | 176665229 | p1.(T)6 |
| 5 | 176665367 | 176665371 | p1.(T)5 |
| 5 | 176671279 | 176671283 | p1.(T)5 |
| 5 | 176673750 | 176673755 | p1.(A)6 |
| 5 | 176673759 | 176673763 | p1.(A)5 |
| 5 | 176675269 | 176675275 | p1.(A)7 |
| 5 | 176678738 | 176678742 | p1.(A)5 |
| 5 | 176694670 | 176694674 | p1.(A)5 |
| 5 | 176696691 | 176696696 | p1.(T)6 |
| 5 | 176696802 | 176696807 | p1.(A)6 |
| 5 | 176700715 | 176700719 | p1.(A)5 |
| 5 | 176707817 | 176707821 | p1.(A)5 |
| 5 | 176707830 | 176707834 | p1.(A)5 |
| 5 | 176707841 | 176707845 | p1.(A)5 |
| 5 | 176710903 | 176710907 | p1.(T)5 |
| 5 | 176721039 | 176721043 | p1.(C)5 |
| 5 | 176721165 | 176721170 | p1.(A)6 |
| 5 | 176721676 | 176721680 | p1.(A)5 |
| 5 | 176721713 | 176721718 | p1.(A)6 |
| 5 | 176722008 | 176722012 | p1.(T)5 |
| 5 | 180039532 | 180039536 | p1.(C)5 |
| 5 | 180043887 | 180043891 | p1.(G)5 |
| 5 | 180046104 | 180046108 | p1.(G)5 |
| 5 | 180047191 | 180047195 | p1.(G)5 |
| 5 | 180047630 | 180047641 | p3.(GAG)4 |
| 5 | 180048013 | 180048017 | p1.(G)5 |
| 5 | 180048255 | 180048259 | p1.(G)5 |
| 5 | 180048638 | 180048642 | p1.(G)5 |
| 5 | 180052994 | 180052998 | p1.(G)5 |
| 5 | 180053023 | 180053029 | p1.(G)7 |
| 5 | 180055897 | 180055902 | p1.(G)6 |
| 5 | 180057717 | 180057721 | p1.(C)5 |
| 5 | 180057786 | 180057790 | p1.(G)5 |
| 5 | 180058748 | 180058754 | p1.(G)7 |
| 6 | 395001 | 395006 | p1.(A)6 |
| 6 | 401582 | 401586 | p1.(A)5 |
| 6 | 401637 | 401641 | p1.(C)5 |
| 6 | 407598 | 407602 | p1.(A)5 |
| 6 | 407623 | 407627 | p1.(T)5 |
| 6 | 407630 | 407648 | p1.(T)19 |
| 6 | 18236684 | 18236688 | p1.(T)5 |
| 6 | 18236771 | 18236775 | p1.(G)5 |
| 6 | 18236786 | 18236790 | p1.(T)5 |
| 6 | 18237613 | 18237618 | p1.(T)6 |
| 6 | 18237685 | 18237690 | p1.(T)6 |
| 6 | 18237718 | 18237722 | p1.(T)5 |
| 6 | 18237737 | 18237742 | p1.(T)6 |
| 6 | 18249928 | 18249932 | p1.(T)5 |
| 6 | 18250027 | 18250031 | p1.(T)5 |
| 6 | 18250049 | 18250055 | p1.(T)7 |
| 6 | 18250077 | 18250081 | p1.(A)5 |
| 6 | 18256579 | 18256583 | p1.(A)5 |
| 6 | 18256592 | 18256596 | p1.(T)5 |
| 6 | 18256613 | 18256617 | p1.(T)5 |
| 6 | 18256633 | 18256637 | p1.(T)5 |
| 6 | 18258171 | 18258175 | p1.(A)5 |
| 6 | 18258250 | 18258256 | p1.(A)7 |
| 6 | 18258300 | 18258310 | p1.(A)11 |
| 6 | 18258596 | 18258601 | p1.(T)6 |
| 6 | 18264066 | 18264071 | p1.(C)6 |
| 6 | 18264075 | 18264079 | p1.(T)5 |
| 6 | 18264079 | 18264096 | p3.(TCC)6 |
| 6 | 18264177 | 18264181 | p1.(G)5 |
| 6 | 18264188 | 18264192 | p1.(C)5 |
| 6 | 26031861 | 26031865 | p1.(A)5 |
| 6 | 26032176 | 26032180 | p1.(T)5 |
| 6 | 27107127 | 27107131 | p1.(G)5 |
| 6 | 28872182 | 28872187 | p1.(G)6 |
| 6 | 28876787 | 28876791 | p1.(T)5 |
| 6 | 28876796 | 28876800 | p1.(A)5 |
| 6 | 28876874 | 28876879 | p1.(A)6 |
| 6 | 28887809 | 28887820 | p3.(GCT)4 |
| 6 | 28889692 | 28889696 | p1.(C)5 |
| 6 | 28889731 | 28889735 | p1.(T)5 |
| 6 | 30153635 | 30153639 | p1.(G)5 |
| 6 | 30153780 | 30153784 | p1.(C)5 |
| 6 | 30153952 | 30153956 | p1.(C)5 |
| 6 | 30154080 | 30154091 | p3.(TCC)4 |
| 6 | 30154114 | 30154118 | p1.(C)5 |
| 6 | 30154228 | 30154232 | p1.(G)5 |
| 6 | 30156985 | 30156990 | p1.(A)6 |
| 6 | 30157254 | 30157261 | p1.(T)8 |
| 6 | 30164529 | 30164533 | p1.(G)5 |
| 6 | 30166295 | 30166300 | p1.(T)6 |
| 6 | 30166308 | 30166312 | p1.(A)5 |
| 6 | 31133481 | 31133485 | p1.(G)5 |
| 6 | 31133713 | 31133717 | p1.(C)5 |
| 6 | 31138182 | 31138187 | p1.(C)6 |
| 6 | 31138204 | 31138209 | p1.(G)6 |
| 6 | 31138213 | 31138217 | p1.(G)5 |
| 6 | 31138221 | 31138225 | p1.(C)5 |
| 6 | 31138247 | 31138251 | p1.(C)5 |
| 6 | 31138326 | 31138331 | p1.(C)6 |
| 6 | 31138357 | 31138361 | p1.(G)5 |
| 6 | 33286806 | 33286811 | p1.(G)6 |
| 6 | 33286921 | 33286925 | p1.(G)5 |
| 6 | 33286952 | 33286956 | p1.(T)5 |
| 6 | 33287204 | 33287209 | p1.(T)6 |
| 6 | 33287213 | 33287218 | p1.(G)6 |
| 6 | 33287477 | 33287481 | p1.(G)5 |
| 6 | 33288181 | 33288185 | p1.(G)5 |
| 6 | 33288230 | 33288235 | p1.(T)6 |
| 6 | 33288505 | 33288509 | p1.(C)5 |
| 6 | 33288636 | 33288640 | p1.(G)5 |
| 6 | 33289130 | 33289134 | p1.(T)5 |
| 6 | 33289253 | 33289257 | p1.(A)5 |
| 6 | 33289715 | 33289719 | p1.(C)5 |
| 6 | 33290632 | 33290636 | p1.(C)5 |
| 6 | 34211287 | 34211292 | p1.(A)6 |
| 6 | 34212738 | 34212742 | p1.(C)5 |
| 6 | 34212786 | 34212790 | p1.(C)5 |
| 6 | 34212897 | 34212902 | p1.(C)6 |
| 6 | 34213026 | 34213030 | p1.(G)5 |
| 6 | 34213286 | 34213290 | p1.(C)5 |
| 6 | 34213350 | 34213354 | p1.(C)5 |
| 6 | 35423683 | 35423687 | p1.(G)5 |
| 6 | 35423707 | 35423711 | p1.(G)5 |
| 6 | 35423781 | 35423785 | p1.(G)5 |
| 6 | 35423794 | 35423799 | p1.(G)6 |
| 6 | 35423814 | 35423819 | p1.(C)6 |
| 6 | 35425715 | 35425721 | p1.(C)7 |
| 6 | 35430614 | 35430618 | p1.(A)5 |
| 6 | 36564525 | 36564529 | p1.(T)5 |
| 6 | 36566617 | 36566621 | p1.(C)5 |
| 6 | 36566670 | 36566675 | p1.(A)6 |
| 6 | 37138536 | 37138540 | p1.(C)5 |
| 6 | 37139227 | 37139231 | p1.(G)5 |
| 6 | 41652400 | 41652404 | p1.(G)5 |
| 6 | 41652536 | 41652540 | p1.(G)5 |
| 6 | 41652557 | 41652561 | p1.(C)5 |
| 6 | 41652638 | 41652642 | p1.(G)5 |
| 6 | 41652678 | 41652682 | p1.(C)5 |
| 6 | 41655721 | 41655725 | p1.(G)5 |
| 6 | 41658516 | 41658520 | p1.(G)5 |
| 6 | 41658662 | 41658666 | p1.(G)5 |
| 6 | 41658784 | 41658788 | p1.(G)5 |
| 6 | 41658830 | 41658850 | p3.(TGC)7 |
| 6 | 41903746 | 41903751 | p1.(G)6 |
| 6 | 41904990 | 41904994 | p1.(A)5 |
| 6 | 41905008 | 41905012 | p1.(T)5 |
| 6 | 41905136 | 41905140 | p1.(A)5 |
| 6 | 41908153 | 41908157 | p1.(T)5 |
| 6 | 41908284 | 41908288 | p1.(G)5 |
| 6 | 41908326 | 41908330 | p1.(G)5 |
| 6 | 41909318 | 41909322 | p1.(C)5 |
| 6 | 43738412 | 43738416 | p1.(C)5 |
| 6 | 43738450 | 43738465 | p4.(GACA)4 |
| 6 | 43738469 | 43738473 | p1.(C)5 |
| 6 | 43738572 | 43738576 | p1.(G)5 |
| 6 | 43738656 | 43738660 | p1.(G)5 |
| 6 | 43738725 | 43738729 | p1.(G)5 |
| 6 | 43738757 | 43738761 | p1.(G)5 |
| 6 | 43742136 | 43742140 | p1.(C)5 |
| 6 | 43745337 | 43745341 | p1.(G)5 |
| 6 | 43748498 | 43748502 | p1.(A)5 |
| 6 | 43748586 | 43748590 | p1.(C)5 |
| 6 | 43749684 | 43749688 | p1.(T)5 |
| 6 | 43749766 | 43749770 | p1.(A)5 |
| 6 | 44216355 | 44216359 | p1.(T)5 |
| 6 | 44218116 | 44218120 | p1.(A)5 |
| 6 | 44218855 | 44218859 | p1.(T)5 |
| 6 | 44218872 | 44218876 | p1.(A)5 |
| 6 | 44219348 | 44219352 | p1.(A)5 |
| 6 | 44219919 | 44219930 | p3.(AGA)4 |
| 6 | 44220917 | 44220921 | p1.(A)5 |
| 6 | 44221284 | 44221289 | p1.(C)6 |
| 6 | 44221427 | 44221431 | p1.(C)5 |
| 6 | 44221447 | 44221453 | p1.(T)7 |
| 6 | 44221611 | 44221615 | p1.(T)5 |
| 6 | 106534376 | 106534380 | p1.(G)5 |
| 6 | 106534448 | 106534452 | p1.(A)5 |
| 6 | 106536080 | 106536084 | p1.(C)5 |
| 6 | 106543600 | 106543604 | p1.(T)5 |
| 6 | 106552738 | 106552742 | p1.(A)5 |
| 6 | 106552960 | 106552964 | p1.(T)5 |
| 6 | 106553015 | 106553019 | p1.(C)5 |
| 6 | 106553281 | 106553286 | p1.(C)6 |
| 6 | 106553392 | 106553396 | p1.(G)5 |
| 6 | 106553654 | 106553658 | p1.(C)5 |
| 6 | 106553686 | 106553690 | p1.(A)5 |
| 6 | 108985249 | 108985253 | p1.(G)5 |
| 6 | 112390553 | 112390557 | p1.(A)5 |
| 6 | 112390591 | 112390595 | p1.(A)5 |
| 6 | 112390640 | 112390644 | p1.(T)5 |
| 6 | 112390836 | 112390840 | p1.(G)5 |
| 6 | 117609742 | 117609746 | p1.(T)5 |
| 6 | 117609971 | 117609977 | p1.(A)7 |
| 6 | 117622205 | 117622210 | p1.(A)6 |
| 6 | 117641139 | 117641143 | p1.(T)5 |
| 6 | 117642528 | 117642533 | p1.(T)6 |
| 6 | 117647482 | 117647486 | p1.(T)5 |
| 6 | 117647582 | 117647587 | p1.(A)6 |
| 6 | 117650558 | 117650562 | p1.(T)5 |
| 6 | 117650618 | 117650624 | p1.(A)7 |
| 6 | 117658508 | 117658512 | p1.(A)5 |
| 6 | 117662483 | 117662488 | p1.(A)6 |
| 6 | 117665251 | 117665255 | p1.(T)5 |
| 6 | 117674324 | 117674328 | p1.(T)5 |
| 6 | 117674338 | 117674342 | p1.(A)5 |
| 6 | 117678986 | 117678990 | p1.(A)5 |
| 6 | 117679149 | 117679153 | p1.(A)5 |
| 6 | 117681120 | 117681126 | p1.(A)7 |
| 6 | 117681183 | 117681189 | p1.(A)7 |
| 6 | 117686799 | 117686803 | p1.(G)5 |
| 6 | 117704481 | 117704485 | p1.(T)5 |
| 6 | 117704674 | 117704679 | p1.(A)6 |
| 6 | 117706969 | 117706973 | p1.(A)5 |
| 6 | 117717435 | 117717439 | p1.(A)5 |
| 6 | 117718143 | 117718147 | p1.(T)5 |
| 6 | 117718150 | 117718154 | p1.(T)5 |
| 6 | 117725598 | 117725603 | p1.(A)6 |
| 6 | 117746838 | 117746842 | p1.(T)5 |
| 6 | 117884426 | 117884431 | p1.(T)6 |
| 6 | 117888010 | 117888014 | p1.(T)5 |
| 6 | 117896328 | 117896333 | p1.(T)6 |
| 6 | 117896482 | 117896487 | p1.(T)6 |
| 6 | 117896489 | 117896494 | p1.(T)6 |
| 6 | 117896524 | 117896528 | p1.(T)5 |
| 6 | 117923382 | 117923386 | p1.(C)5 |
| 6 | 117923402 | 117923406 | p1.(C)5 |
| 6 | 117923411 | 117923415 | p1.(C)5 |
| 6 | 135513546 | 135513557 | p4.(AGCC)3 |
| 6 | 135516873 | 135516877 | p1.(C)5 |
| 6 | 135520107 | 135520112 | p1.(C)6 |
| 6 | 135521214 | 135521218 | p1.(T)5 |
| 6 | 135524433 | 135524437 | p1.(A)5 |
| 6 | 138192450 | 138192454 | p1.(T)5 |
| 6 | 138196178 | 138196187 | p1.(T)5(C)5 |
| 6 | 138198200 | 138198206 | p1.(T)7 |
| 6 | 138198275 | 138198279 | p1.(T)5 |
| 6 | 138199945 | 138199950 | p1.(G)6 |
| 6 | 138202341 | 138202345 | p1.(C)5 |
| 6 | 138202351 | 138202357 | p1.(C)7 |
| 6 | 138202374 | 138202378 | p1.(C)5 |
| 6 | 139135617 | 139135621 | p1.(C)5 |
| 6 | 139135710 | 139135716 | p1.(T)7 |
| 6 | 139159482 | 139159486 | p1.(T)5 |
| 6 | 139159583 | 139159587 | p1.(T)5 |
| 6 | 139159637 | 139159641 | p1.(T)5 |
| 6 | 139165541 | 139165545 | p1.(T)5 |
| 6 | 139165569 | 139165573 | p1.(C)5 |
| 6 | 139167719 | 139167723 | p1.(A)5 |
| 6 | 139167740 | 139167744 | p1.(A)5 |
| 6 | 139170491 | 139170495 | p1.(A)5 |
| 6 | 139175149 | 139175156 | p1.(T)8 |
| 6 | 139175180 | 139175184 | p1.(A)5 |
| 6 | 139175252 | 139175256 | p1.(G)5 |
| 6 | 139183821 | 139183825 | p1.(C)5 |
| 6 | 139206941 | 139206946 | p1.(A)6 |
| 6 | 139207980 | 139207986 | p1.(T)7 |
| 6 | 139222184 | 139222188 | p1.(A)5 |
| 6 | 152129146 | 152129150 | p1.(C)5 |
| 6 | 152129329 | 152129334 | p1.(G)6 |
| 6 | 152129338 | 152129343 | p1.(C)6 |
| 6 | 152163717 | 152163729 | p1.(T)7(C)6 |
| 6 | 152201837 | 152201841 | p1.(A)5 |
| 6 | 152265517 | 152265521 | p1.(C)5 |
| 6 | 152415641 | 152415652 | p3.(GCA)4 |
| 6 | 152420068 | 152420072 | p1.(G)5 |
| 6 | 159188461 | 159188466 | p1.(G)6 |
| 6 | 159188471 | 159188475 | p1.(G)5 |
| 6 | 159188477 | 159188481 | p1.(G)5 |
| 6 | 159191877 | 159191886 | p2.(TC)5 |
| 6 | 159197474 | 159197478 | p1.(T)5 |
| 6 | 159197545 | 159197549 | p1.(A)5 |
| 6 | 159204621 | 159204626 | p1.(T)6 |
| 6 | 159206452 | 159206457 | p1.(G)6 |
| 6 | 159208242 | 159208253 | p2.(GA)6 |
| 6 | 159210326 | 159210330 | p1.(A)5 |
| 6 | 159210407 | 159210411 | p1.(A)5 |
| 6 | 167417867 | 167417871 | p1.(A)5 |
| 6 | 167424351 | 167424355 | p1.(A)5 |
| 6 | 167435989 | 167435993 | p1.(A)5 |
| 6 | 167438322 | 167438327 | p1.(C)6 |
| 6 | 167438330 | 167438334 | p1.(A)5 |
| 6 | 167446136 | 167446140 | p1.(A)5 |
| 6 | 167453520 | 167453531 | p1.(T)12 |
| 6 | 167453584 | 167453588 | p1.(A)5 |
| 6 | 167453600 | 167453605 | p1.(T)6 |
| 6 | 168227739 | 168227743 | p1.(C)5 |
| 6 | 168275999 | 168276007 | p1.(T)9 |
| 6 | 168276119 | 168276123 | p1.(A)5 |
| 6 | 168281031 | 168281036 | p1.(T)6 |
| 6 | 168281163 | 168281167 | p1.(A)5 |
| 6 | 168289886 | 168289892 | p1.(T)7 |
| 6 | 168289954 | 168289965 | p3.(TGA)4 |
| 6 | 168299094 | 168299098 | p1.(A)5 |
| 6 | 168311975 | 168311979 | p1.(T)5 |
| 6 | 168315411 | 168315415 | p1.(A)5 |
| 6 | 168319589 | 168319593 | p1.(T)5 |
| 6 | 168325729 | 168325734 | p1.(A)6 |
| 6 | 168343797 | 168343801 | p1.(T)5 |
| 6 | 168344722 | 168344726 | p1.(C)5 |
| 6 | 168347542 | 168347546 | p1.(A)5 |
| 6 | 168348628 | 168348639 | p4.(TGAT)3 |
| 6 | 168351857 | 168351861 | p1.(T)5 |
| 6 | 168352157 | 168352161 | p1.(C)5 |
| 6 | 168352226 | 168352230 | p1.(C)5 |
| 6 | 168352576 | 168352580 | p1.(G)5 |
| 6 | 168352590 | 168352594 | p1.(C)5 |
| 6 | 168352796 | 168352807 | p3.(GAG)4 |
| 6 | 168352873 | 168352878 | p1.(G)6 |
| 7 | 2956939 | 2956943 | p1.(A)5 |
| 7 | 2956965 | 2956969 | p1.(G)5 |
| 7 | 2956977 | 2956981 | p1.(G)5 |
| 7 | 2959196 | 2959200 | p1.(C)5 |
| 7 | 2962854 | 2962859 | p1.(C)6 |
| 7 | 2963941 | 2963955 | p3.(GGA)5 |
| 7 | 2968283 | 2968287 | p1.(G)5 |
| 7 | 2968323 | 2968329 | p1.(G)7 |
| 7 | 2972200 | 2972204 | p1.(G)5 |
| 7 | 2979408 | 2979412 | p1.(T)5 |
| 7 | 6029421 | 6029425 | p1.(T)5 |
| 7 | 6029594 | 6029599 | p1.(A)6 |
| 7 | 6035199 | 6035203 | p1.(A)5 |
| 7 | 6035204 | 6035215 | p4.(CTGT)3 |
| 7 | 6038913 | 6038918 | p1.(A)6 |
| 7 | 6042170 | 6042174 | p1.(G)5 |
| 7 | 6042177 | 6042181 | p1.(G)5 |
| 7 | 6043349 | 6043354 | p1.(C)6 |
| 7 | 6431651 | 6431655 | p1.(C)5 |
| 7 | 6439748 | 6439752 | p1.(T)5 |
| 7 | 13935462 | 13935466 | p1.(A)5 |
| 7 | 13935520 | 13935524 | p1.(G)5 |
| 7 | 13935534 | 13935538 | p1.(C)5 |
| 7 | 13940436 | 13940440 | p1.(T)5 |
| 7 | 13946143 | 13946147 | p1.(A)5 |
| 7 | 13971174 | 13971178 | p1.(G)5 |
| 7 | 13975473 | 13975479 | p1.(G)7 |
| 7 | 13978876 | 13978881 | p1.(A)6 |
| 7 | 26232161 | 26232165 | p1.(C)5 |
| 7 | 26232885 | 26232890 | p1.(C)6 |
| 7 | 26232999 | 26233004 | p1.(A)6 |
| 7 | 26233227 | 26233231 | p1.(C)5 |
| 7 | 26236287 | 26236291 | p1.(A)5 |
| 7 | 26236526 | 26236530 | p1.(T)5 |
| 7 | 26237027 | 26237031 | p1.(A)5 |
| 7 | 27203196 | 27203201 | p1.(T)6 |
| 7 | 27203415 | 27203419 | p1.(T)5 |
| 7 | 27204507 | 27204512 | p1.(G)6 |
| 7 | 27204573 | 27204577 | p1.(T)5 |
| 7 | 27204771 | 27204782 | p3.(CGC)4 |
| 7 | 27204794 | 27204798 | p1.(G)5 |
| 7 | 27222386 | 27222391 | p1.(C)6 |
| 7 | 27222462 | 27222470 | p1.(T)9 |
| 7 | 27222626 | 27222630 | p1.(T)5 |
| 7 | 27224255 | 27224259 | p1.(G)5 |
| 7 | 27224333 | 27224337 | p1.(A)5 |
| 7 | 27224404 | 27224408 | p1.(G)5 |
| 7 | 27224538 | 27224542 | p1.(G)5 |
| 7 | 27237806 | 27237810 | p1.(T)5 |
| 7 | 27237847 | 27237852 | p1.(T)6 |
| 7 | 27238018 | 27238022 | p1.(C)5 |
| 7 | 27238918 | 27238922 | p1.(C)5 |
| 7 | 27239082 | 27239099 | p3.(GGC)6 |
| 7 | 27239299 | 27239316 | p3.(GCG)6 |
| 7 | 27239356 | 27239360 | p1.(G)5 |
| 7 | 27239370 | 27239374 | p1.(G)5 |
| 7 | 27239470 | 27239481 | p3.(GCC)4 |
| 7 | 27239494 | 27239498 | p1.(C)5 |
| 7 | 27239519 | 27239523 | p1.(G)5 |
| 7 | 27239527 | 27239531 | p1.(C)5 |
| 7 | 27239533 | 27239537 | p1.(C)5 |
| 7 | 27239575 | 27239586 | p3.(GCC)4 |
| 7 | 27239669 | 27239673 | p1.(G)5 |
| 7 | 27872456 | 27872460 | p1.(G)5 |
| 7 | 27880327 | 27880331 | p1.(T)5 |
| 7 | 27934870 | 27934874 | p1.(G)5 |
| 7 | 27934891 | 27934896 | p1.(G)6 |
| 7 | 27934922 | 27934926 | p1.(G)5 |
| 7 | 28031575 | 28031579 | p1.(T)5 |
| 7 | 28031608 | 28031612 | p1.(A)5 |
| 7 | 28220144 | 28220148 | p1.(C)5 |
| 7 | 28220213 | 28220218 | p1.(C)6 |
| 7 | 28220249 | 28220264 | p4.(GAGG)4 |
| 7 | 41729229 | 41729233 | p1.(C)5 |
| 7 | 41729291 | 41729295 | p1.(T)5 |
| 7 | 41729717 | 41729721 | p1.(T)5 |
| 7 | 41729727 | 41729731 | p1.(C)5 |
| 7 | 41729741 | 41729752 | p3.(TTC)4 |
| 7 | 41729902 | 41729906 | p1.(T)5 |
| 7 | 41729931 | 41729935 | p1.(C)5 |
| 7 | 41739874 | 41739878 | p1.(G)5 |
| 7 | 41739907 | 41739911 | p1.(G)5 |
| 7 | 50367244 | 50367249 | p1.(C)6 |
| 7 | 50467731 | 50467735 | p1.(G)5 |
| 7 | 50467765 | 50467769 | p1.(C)5 |
| 7 | 50468338 | 50468342 | p1.(C)5 |
| 7 | 55086911 | 55086915 | p1.(C)5 |
| 7 | 55209967 | 55209971 | p1.(T)5 |
| 7 | 55220231 | 55220235 | p1.(T)5 |
| 7 | 55220290 | 55220294 | p1.(C)5 |
| 7 | 55220336 | 55220340 | p1.(C)5 |
| 7 | 55221748 | 55221753 | p1.(C)6 |
| 7 | 55224297 | 55224301 | p1.(A)5 |
| 7 | 55224349 | 55224353 | p1.(G)5 |
| 7 | 55225358 | 55225362 | p1.(T)5 |
| 7 | 55227932 | 55227936 | p1.(A)5 |
| 7 | 55227965 | 55227970 | p1.(A)6 |
| 7 | 55229225 | 55229229 | p1.(C)5 |
| 7 | 55233014 | 55233018 | p1.(C)5 |
| 7 | 55240709 | 55240713 | p1.(G)5 |
| 7 | 55241605 | 55241611 | p1.(C)7 |
| 7 | 55241689 | 55241693 | p1.(A)5 |
| 7 | 55248972 | 55248983 | p4.(CTCC)3 |
| 7 | 55249015 | 55249019 | p1.(C)5 |
| 7 | 55268082 | 55268086 | p1.(C)5 |
| 7 | 55268880 | 55268884 | p1.(G)5 |
| 7 | 55269420 | 55269424 | p1.(T)5 |
| 7 | 55273058 | 55273062 | p1.(C)5 |
| 7 | 66453434 | 66453439 | p1.(T)6 |
| 7 | 66458407 | 66458413 | p1.(A)7 |
| 7 | 66459254 | 66459258 | p1.(T)5 |
| 7 | 66460264 | 66460268 | p1.(G)5 |
| 7 | 66460304 | 66460308 | p1.(T)5 |
| 7 | 66460387 | 66460391 | p1.(G)5 |
| 7 | 73450875 | 73450880 | p1.(C)6 |
| 7 | 73456975 | 73456979 | p1.(G)5 |
| 7 | 73459542 | 73459546 | p1.(C)5 |
| 7 | 73459571 | 73459575 | p1.(G)5 |
| 7 | 73461040 | 73461044 | p1.(G)5 |
| 7 | 73461083 | 73461087 | p1.(C)5 |
| 7 | 73462826 | 73462830 | p1.(C)5 |
| 7 | 73462840 | 73462844 | p1.(C)5 |
| 7 | 73462847 | 73462858 | p3.(GCA)4 |
| 7 | 73470654 | 73470658 | p1.(G)5 |
| 7 | 73472033 | 73472037 | p1.(C)5 |
| 7 | 73477649 | 73477660 | p3.(CCG)4 |
| 7 | 73478013 | 73478017 | p1.(G)5 |
| 7 | 73480300 | 73480304 | p1.(G)5 |
| 7 | 75167499 | 75167503 | p1.(T)5 |
| 7 | 75168684 | 75168688 | p1.(T)5 |
| 7 | 75168704 | 75168708 | p1.(T)5 |
| 7 | 75177117 | 75177121 | p1.(A)5 |
| 7 | 75178254 | 75178258 | p1.(C)5 |
| 7 | 75184819 | 75184823 | p1.(T)5 |
| 7 | 75184858 | 75184862 | p1.(A)5 |
| 7 | 75187006 | 75187011 | p1.(T)6 |
| 7 | 75189224 | 75189229 | p1.(G)6 |
| 7 | 75192296 | 75192300 | p1.(G)5 |
| 7 | 75192503 | 75192507 | p1.(G)5 |
| 7 | 75221837 | 75221843 | p1.(G)7 |
| 7 | 75228512 | 75228516 | p1.(T)5 |
| 7 | 75228569 | 75228573 | p1.(A)5 |
| 7 | 75368106 | 75368110 | p1.(C)5 |
| 7 | 81331966 | 81331970 | p1.(A)5 |
| 7 | 81332070 | 81332074 | p1.(C)5 |
| 7 | 81334699 | 81334703 | p1.(T)5 |
| 7 | 81336593 | 81336597 | p1.(A)5 |
| 7 | 81336686 | 81336690 | p1.(A)5 |
| 7 | 81350139 | 81350143 | p1.(T)5 |
| 7 | 81359104 | 81359109 | p1.(A)6 |
| 7 | 81381504 | 81381508 | p1.(C)5 |
| 7 | 81381573 | 81381577 | p1.(A)5 |
| 7 | 81388045 | 81388050 | p1.(T)6 |
| 7 | 81388108 | 81388112 | p1.(A)5 |
| 7 | 81388124 | 81388130 | p1.(A)7 |
| 7 | 81392088 | 81392093 | p1.(T)6 |
| 7 | 81392096 | 81392100 | p1.(T)5 |
| 7 | 81392145 | 81392150 | p1.(T)6 |
| 7 | 91603014 | 91603019 | p1.(T)6 |
| 7 | 91603085 | 91603092 | p1.(A)8 |
| 7 | 91603096 | 91603100 | p1.(A)5 |
| 7 | 91621461 | 91621465 | p1.(T)5 |
| 7 | 91625049 | 91625053 | p1.(A)5 |
| 7 | 91643633 | 91643638 | p1.(T)6 |
| 7 | 91651624 | 91651628 | p1.(T)5 |
| 7 | 91652170 | 91652175 | p1.(A)6 |
| 7 | 91659279 | 91659283 | p1.(A)5 |
| 7 | 91659314 | 91659318 | p1.(T)5 |
| 7 | 91667790 | 91667794 | p1.(G)5 |
| 7 | 91671392 | 91671403 | p2.(AG)6 |
| 7 | 91682235 | 91682239 | p1.(A)5 |
| 7 | 91691612 | 91691617 | p1.(A)6 |
| 7 | 91691621 | 91691625 | p1.(T)5 |
| 7 | 91691642 | 91691647 | p1.(A)6 |
| 7 | 91699372 | 91699377 | p1.(A)6 |
| 7 | 91706997 | 91707001 | p1.(T)5 |
| 7 | 91707110 | 91707114 | p1.(A)5 |
| 7 | 91708403 | 91708409 | p1.(A)7 |
| 7 | 91708517 | 91708521 | p1.(A)5 |
| 7 | 91708684 | 91708688 | p1.(A)5 |
| 7 | 91709093 | 91709097 | p1.(A)5 |
| 7 | 91709321 | 91709325 | p1.(A)5 |
| 7 | 91709345 | 91709351 | p1.(A)7 |
| 7 | 91709360 | 91709364 | p1.(A)5 |
| 7 | 91709410 | 91709415 | p1.(A)6 |
| 7 | 91709425 | 91709439 | p5.(AAAGA)3 |
| 7 | 91711875 | 91711880 | p1.(T)6 |
| 7 | 91711954 | 91711958 | p1.(A)5 |
| 7 | 91712575 | 91712579 | p1.(A)5 |
| 7 | 91712607 | 91712611 | p1.(A)5 |
| 7 | 91712721 | 91712725 | p1.(A)5 |
| 7 | 91712800 | 91712805 | p1.(A)6 |
| 7 | 91712978 | 91712982 | p1.(T)5 |
| 7 | 91724401 | 91724406 | p1.(A)6 |
| 7 | 91726097 | 91726102 | p1.(A)6 |
| 7 | 91726334 | 91726338 | p1.(A)5 |
| 7 | 91726373 | 91726377 | p1.(A)5 |
| 7 | 91726415 | 91726419 | p1.(A)5 |
| 7 | 91726480 | 91726486 | p1.(A)7 |
| 7 | 91726628 | 91726632 | p1.(A)5 |
| 7 | 91727411 | 91727415 | p1.(T)5 |
| 7 | 91727536 | 91727540 | p1.(T)5 |
| 7 | 91728992 | 91728996 | p1.(T)5 |
| 7 | 91732039 | 91732045 | p1.(G)7 |
| 7 | 91734984 | 91734989 | p1.(T)6 |
| 7 | 91734999 | 91735003 | p1.(T)5 |
| 7 | 92244391 | 92244395 | p1.(G)5 |
| 7 | 92247376 | 92247380 | p1.(A)5 |
| 7 | 92300790 | 92300794 | p1.(G)5 |
| 7 | 92404153 | 92404158 | p1.(A)6 |
| 7 | 98478770 | 98478774 | p1.(A)5 |
| 7 | 98478794 | 98478798 | p1.(G)5 |
| 7 | 98478828 | 98478832 | p1.(A)5 |
| 7 | 98488076 | 98488082 | p1.(A)7 |
| 7 | 98490033 | 98490038 | p1.(T)6 |
| 7 | 98490115 | 98490120 | p1.(A)6 |
| 7 | 98490143 | 98490147 | p1.(T)5 |
| 7 | 98491411 | 98491418 | p1.(T)8 |
| 7 | 98493378 | 98493384 | p1.(T)7 |
| 7 | 98493449 | 98493453 | p1.(T)5 |
| 7 | 98495412 | 98495416 | p1.(C)5 |
| 7 | 98498314 | 98498318 | p1.(T)5 |
| 7 | 98507728 | 98507732 | p1.(T)5 |
| 7 | 98507853 | 98507857 | p1.(C)5 |
| 7 | 98507859 | 98507863 | p1.(C)5 |
| 7 | 98507865 | 98507869 | p1.(C)5 |
| 7 | 98507893 | 98507897 | p1.(C)5 |
| 7 | 98508876 | 98508881 | p1.(A)6 |
| 7 | 98515033 | 98515037 | p1.(C)5 |
| 7 | 98524998 | 98525002 | p1.(G)5 |
| 7 | 98528336 | 98528341 | p1.(G)6 |
| 7 | 98530881 | 98530886 | p1.(C)6 |
| 7 | 98531029 | 98531034 | p1.(G)6 |
| 7 | 98535262 | 98535267 | p1.(T)6 |
| 7 | 98535273 | 98535277 | p1.(T)5 |
| 7 | 98540560 | 98540564 | p1.(T)5 |
| 7 | 98540617 | 98540621 | p1.(G)5 |
| 7 | 98545875 | 98545879 | p1.(T)5 |
| 7 | 98547042 | 98547047 | p1.(T)6 |
| 7 | 98547123 | 98547128 | p1.(G)6 |
| 7 | 98547301 | 98547305 | p1.(A)5 |
| 7 | 98547796 | 98547800 | p1.(A)5 |
| 7 | 98548487 | 98548494 | p1.(T)8 |
| 7 | 98548546 | 98548550 | p1.(G)5 |
| 7 | 98550875 | 98550880 | p1.(C)6 |
| 7 | 98552722 | 98552726 | p1.(T)5 |
| 7 | 98554014 | 98554018 | p1.(T)5 |
| 7 | 98563430 | 98563434 | p1.(A)5 |
| 7 | 98564719 | 98564723 | p1.(A)5 |
| 7 | 98564757 | 98564761 | p1.(T)5 |
| 7 | 98565202 | 98565206 | p1.(T)5 |
| 7 | 98565212 | 98565216 | p1.(T)5 |
| 7 | 98567763 | 98567767 | p1.(C)5 |
| 7 | 98575825 | 98575831 | p1.(T)7 |
| 7 | 98575870 | 98575875 | p1.(A)6 |
| 7 | 98581816 | 98581820 | p1.(A)5 |
| 7 | 98581964 | 98581968 | p1.(A)5 |
| 7 | 98582553 | 98582560 | p1.(T)8 |
| 7 | 98588108 | 98588112 | p1.(A)5 |
| 7 | 98592295 | 98592300 | p1.(C)6 |
| 7 | 98601805 | 98601809 | p1.(T)5 |
| 7 | 98601869 | 98601873 | p1.(A)5 |
| 7 | 98602962 | 98602966 | p1.(T)5 |
| 7 | 98609745 | 98609751 | p1.(A)7 |
| 7 | 106508725 | 106508729 | p1.(C)5 |
| 7 | 106508868 | 106508872 | p1.(A)5 |
| 7 | 106509148 | 106509152 | p1.(T)5 |
| 7 | 106509352 | 106509356 | p1.(C)5 |
| 7 | 106513176 | 106513180 | p1.(T)5 |
| 7 | 106513324 | 106513328 | p1.(A)5 |
| 7 | 106519968 | 106519972 | p1.(A)5 |
| 7 | 106519994 | 106519999 | p1.(A)6 |
| 7 | 106523586 | 106523590 | p1.(A)5 |
| 7 | 106545698 | 106545702 | p1.(A)5 |
| 7 | 106545716 | 106545720 | p1.(A)5 |
| 7 | 106545850 | 106545854 | p1.(A)5 |
| 7 | 116339146 | 116339150 | p1.(C)5 |
| 7 | 116339820 | 116339824 | p1.(T)5 |
| 7 | 116340050 | 116340055 | p1.(A)6 |
| 7 | 116340276 | 116340280 | p1.(A)5 |
| 7 | 116340302 | 116340306 | p1.(T)5 |
| 7 | 116397479 | 116397486 | p1.(T)8 |
| 7 | 116397808 | 116397812 | p1.(A)5 |
| 7 | 116397823 | 116397827 | p1.(A)5 |
| 7 | 116398499 | 116398506 | p1.(T)8 |
| 7 | 116399473 | 116399477 | p1.(A)5 |
| 7 | 116403178 | 116403182 | p1.(C)5 |
| 7 | 116403197 | 116403201 | p1.(T)5 |
| 7 | 116409676 | 116409690 | p1.(T)15 |
| 7 | 116411545 | 116411549 | p1.(T)5 |
| 7 | 116411672 | 116411676 | p1.(T)5 |
| 7 | 116411687 | 116411691 | p1.(A)5 |
| 7 | 116411714 | 116411718 | p1.(T)5 |
| 7 | 116415001 | 116415005 | p1.(C)5 |
| 7 | 116422111 | 116422115 | p1.(A)5 |
| 7 | 116423366 | 116423370 | p1.(A)5 |
| 7 | 116435929 | 116435933 | p1.(T)5 |
| 7 | 124462469 | 124462473 | p1.(A)5 |
| 7 | 124462616 | 124462621 | p1.(A)6 |
| 7 | 124475297 | 124475326 | p5.(AAACA)6 |
| 7 | 124481074 | 124481078 | p1.(T)5 |
| 7 | 124481106 | 124481110 | p1.(T)5 |
| 7 | 124481113 | 124481117 | p1.(T)5 |
| 7 | 124481129 | 124481133 | p1.(T)5 |
| 7 | 124481193 | 124481197 | p1.(A)5 |
| 7 | 124482959 | 124482963 | p1.(T)5 |
| 7 | 124493026 | 124493030 | p1.(T)5 |
| 7 | 124493195 | 124493200 | p1.(A)6 |
| 7 | 124503391 | 124503396 | p1.(A)6 |
| 7 | 124503678 | 124503682 | p1.(T)5 |
| 7 | 124510996 | 124511000 | p1.(T)5 |
| 7 | 124532340 | 124532344 | p1.(G)5 |
| 7 | 124532410 | 124532419 | p2.(TA)5 |
| 7 | 128828933 | 128828938 | p1.(G)6 |
| 7 | 128828954 | 128828958 | p1.(G)5 |
| 7 | 128829015 | 128829019 | p1.(G)5 |
| 7 | 128829040 | 128829060 | p3.(GCT)7 |
| 7 | 128829061 | 128829065 | p1.(G)5 |
| 7 | 128829075 | 128829080 | p1.(G)6 |
| 7 | 128829195 | 128829199 | p1.(C)5 |
| 7 | 128843218 | 128843222 | p1.(C)5 |
| 7 | 128843237 | 128843242 | p1.(C)6 |
| 7 | 128845540 | 128845551 | p4.(TGGC)3 |
| 7 | 128849133 | 128849137 | p1.(T)5 |
| 7 | 128851867 | 128851871 | p1.(C)5 |
| 7 | 128851990 | 128851994 | p1.(C)5 |
| 7 | 128852004 | 128852009 | p1.(C)6 |
| 7 | 128852018 | 128852022 | p1.(C)5 |
| 7 | 128852155 | 128852159 | p1.(C)5 |
| 7 | 128852189 | 128852193 | p1.(C)5 |
| 7 | 137565203 | 137565207 | p1.(G)5 |
| 7 | 137567290 | 137567295 | p1.(C)6 |
| 7 | 137567332 | 137567336 | p1.(G)5 |
| 7 | 137588705 | 137588709 | p1.(T)5 |
| 7 | 137593037 | 137593041 | p1.(G)5 |
| 7 | 137593187 | 137593192 | p1.(A)6 |
| 7 | 137600671 | 137600675 | p1.(G)5 |
| 7 | 137600766 | 137600771 | p1.(A)6 |
| 7 | 138522806 | 138522810 | p1.(G)5 |
| 7 | 138522909 | 138522913 | p1.(A)5 |
| 7 | 138524926 | 138524931 | p1.(C)6 |
| 7 | 138529070 | 138529074 | p1.(C)5 |
| 7 | 138529172 | 138529176 | p1.(G)5 |
| 7 | 138545927 | 138545931 | p1.(C)5 |
| 7 | 138546032 | 138546036 | p1.(G)5 |
| 7 | 138552754 | 138552759 | p1.(G)6 |
| 7 | 138552883 | 138552887 | p1.(A)5 |
| 7 | 138556027 | 138556032 | p1.(G)6 |
| 7 | 138591757 | 138591761 | p1.(A)5 |
| 7 | 138601643 | 138601648 | p1.(G)6 |
| 7 | 138601804 | 138601808 | p1.(G)5 |
| 7 | 138602056 | 138602061 | p1.(G)6 |
| 7 | 138602382 | 138602386 | p1.(A)5 |
| 7 | 138602401 | 138602405 | p1.(G)5 |
| 7 | 138602480 | 138602484 | p1.(G)5 |
| 7 | 138602492 | 138602496 | p1.(A)5 |
| 7 | 138602538 | 138602542 | p1.(A)5 |
| 7 | 138602605 | 138602609 | p1.(A)5 |
| 7 | 138602642 | 138602646 | p1.(T)5 |
| 7 | 138602699 | 138602703 | p1.(A)5 |
| 7 | 138602804 | 138602808 | p1.(G)5 |
| 7 | 138603456 | 138603460 | p1.(C)5 |
| 7 | 138603524 | 138603530 | p1.(A)7 |
| 7 | 138604003 | 138604008 | p1.(A)6 |
| 7 | 140434422 | 140434426 | p1.(C)5 |
| 7 | 140434441 | 140434445 | p1.(T)5 |
| 7 | 140434528 | 140434532 | p1.(T)5 |
| 7 | 140434575 | 140434585 | p1.(A)11 |
| 7 | 140439643 | 140439647 | p1.(T)5 |
| 7 | 140439738 | 140439742 | p1.(A)5 |
| 7 | 140449187 | 140449191 | p1.(T)5 |
| 7 | 140453161 | 140453165 | p1.(T)5 |
| 7 | 140454037 | 140454041 | p1.(A)5 |
| 7 | 140477809 | 140477813 | p1.(T)5 |
| 7 | 140481500 | 140481504 | p1.(A)5 |
| 7 | 140482927 | 140482933 | p1.(G)7 |
| 7 | 140494272 | 140494276 | p1.(A)5 |
| 7 | 140501344 | 140501348 | p1.(A)5 |
| 7 | 140501351 | 140501355 | p1.(T)5 |
| 7 | 140507866 | 140507870 | p1.(A)5 |
| 7 | 140534451 | 140534455 | p1.(T)5 |
| 7 | 140534499 | 140534503 | p1.(A)5 |
| 7 | 140534585 | 140534589 | p1.(A)5 |
| 7 | 140550015 | 140550020 | p1.(A)6 |
| 7 | 140624550 | 140624554 | p1.(G)5 |
| 7 | 148504717 | 148504722 | p1.(G)6 |
| 7 | 148506171 | 148506176 | p1.(A)6 |
| 7 | 148506210 | 148506214 | p1.(A)5 |
| 7 | 148507509 | 148507513 | p1.(A)5 |
| 7 | 148508745 | 148508749 | p1.(T)5 |
| 7 | 148508768 | 148508772 | p1.(A)5 |
| 7 | 148508774 | 148508778 | p1.(C)5 |
| 7 | 148511052 | 148511056 | p1.(T)5 |
| 7 | 148511094 | 148511098 | p1.(T)5 |
| 7 | 148512036 | 148512040 | p1.(A)5 |
| 7 | 148513791 | 148513795 | p1.(T)5 |
| 7 | 148514998 | 148515009 | p3.(TCT)4 |
| 7 | 148515025 | 148515030 | p1.(C)6 |
| 7 | 148515090 | 148515094 | p1.(G)5 |
| 7 | 148523667 | 148523671 | p1.(G)5 |
| 7 | 148524312 | 148524316 | p1.(A)5 |
| 7 | 148525889 | 148525900 | p3.(CAT)4 |
| 7 | 148525944 | 148525948 | p1.(A)5 |
| 7 | 148526849 | 148526854 | p1.(T)6 |
| 7 | 148526948 | 148526954 | p1.(A)7 |
| 7 | 148543694 | 148543704 | p1.(A)11 |
| 7 | 151836879 | 151836886 | p1.(A)8 |
| 7 | 151843775 | 151843780 | p1.(T)6 |
| 7 | 151845524 | 151845529 | p1.(A)6 |
| 7 | 151845959 | 151845964 | p1.(T)6 |
| 7 | 151846107 | 151846111 | p1.(G)5 |
| 7 | 151846132 | 151846136 | p1.(G)5 |
| 7 | 151847998 | 151848002 | p1.(T)5 |
| 7 | 151851236 | 151851240 | p1.(A)5 |
| 7 | 151851386 | 151851390 | p1.(G)5 |
| 7 | 151851534 | 151851539 | p1.(A)6 |
| 7 | 151853400 | 151853404 | p1.(G)5 |
| 7 | 151856009 | 151856013 | p1.(T)5 |
| 7 | 151856100 | 151856104 | p1.(T)5 |
| 7 | 151856145 | 151856149 | p1.(C)5 |
| 7 | 151859283 | 151859287 | p1.(T)5 |
| 7 | 151859308 | 151859312 | p1.(T)5 |
| 7 | 151859314 | 151859318 | p1.(T)5 |
| 7 | 151859851 | 151859855 | p1.(T)5 |
| 7 | 151859859 | 151859863 | p1.(T)5 |
| 7 | 151860074 | 151860078 | p1.(A)5 |
| 7 | 151860578 | 151860582 | p1.(T)5 |
| 7 | 151860601 | 151860605 | p1.(G)5 |
| 7 | 151864470 | 151864474 | p1.(A)5 |
| 7 | 151873588 | 151873593 | p1.(A)6 |
| 7 | 151873715 | 151873726 | p3.(TGG)4 |
| 7 | 151874013 | 151874019 | p1.(T)7 |
| 7 | 151874068 | 151874072 | p1.(T)5 |
| 7 | 151874093 | 151874098 | p1.(T)6 |
| 7 | 151874148 | 151874156 | p1.(T)9 |
| 7 | 151874419 | 151874423 | p1.(C)5 |
| 7 | 151874474 | 151874478 | p1.(T)5 |
| 7 | 151874914 | 151874918 | p1.(A)5 |
| 7 | 151875101 | 151875127 | p1.(A)27 |
| 7 | 151877054 | 151877058 | p1.(G)5 |
| 7 | 151878109 | 151878113 | p1.(G)5 |
| 7 | 151878541 | 151878545 | p1.(G)5 |
| 7 | 151878596 | 151878600 | p1.(A)5 |
| 7 | 151878689 | 151878693 | p1.(A)5 |
| 7 | 151879202 | 151879206 | p1.(A)5 |
| 7 | 151879244 | 151879248 | p1.(T)5 |
| 7 | 151879594 | 151879608 | p3.(TGC)5 |
| 7 | 151879612 | 151879616 | p1.(T)5 |
| 7 | 151880046 | 151880050 | p1.(A)5 |
| 7 | 151880116 | 151880120 | p1.(A)5 |
| 7 | 151880236 | 151880240 | p1.(T)5 |
| 7 | 151884553 | 151884557 | p1.(T)5 |
| 7 | 151884567 | 151884572 | p1.(A)6 |
| 7 | 151884924 | 151884928 | p1.(A)5 |
| 7 | 151884941 | 151884947 | p1.(A)7 |
| 7 | 151891126 | 151891130 | p1.(G)5 |
| 7 | 151900053 | 151900057 | p1.(T)5 |
| 7 | 151900060 | 151900064 | p1.(T)5 |
| 7 | 151900081 | 151900086 | p1.(T)6 |
| 7 | 151902317 | 151902322 | p1.(A)6 |
| 7 | 151945668 | 151945672 | p1.(T)5 |
| 7 | 151948058 | 151948063 | p1.(A)6 |
| 7 | 152009035 | 152009049 | p5.(AAAAT)3 |
| 7 | 152012386 | 152012391 | p1.(T)6 |
| 7 | 152012404 | 152012408 | p1.(A)5 |
| 7 | 152012429 | 152012433 | p1.(A)5 |
| 7 | 152132815 | 152132820 | p1.(G)6 |
| 7 | 152132914 | 152132919 | p1.(G)6 |
| 7 | 156798245 | 156798249 | p1.(G)5 |
| 7 | 156798464 | 156798468 | p1.(C)5 |
| 7 | 156798518 | 156798522 | p1.(T)5 |
| 7 | 156802527 | 156802541 | p3.(GCC)5 |
| 7 | 156802899 | 156802913 | p3.(CCG)5 |
| 7 | 156802932 | 156802943 | p3.(GCG)4 |
| 7 | 156802992 | 156802997 | p1.(G)6 |
| 7 | 156803028 | 156803032 | p1.(T)5 |
| 7 | 156803036 | 156803040 | p1.(T)5 |
| 7 | 156803134 | 156803138 | p1.(G)5 |
| 8 | 17796333 | 17796337 | p1.(T)5 |
| 8 | 17796383 | 17796388 | p1.(C)6 |
| 8 | 17813153 | 17813157 | p1.(A)5 |
| 8 | 17815108 | 17815119 | p3.(GAG)4 |
| 8 | 17817883 | 17817894 | p4.(AAGA)3 |
| 8 | 17819536 | 17819540 | p1.(T)5 |
| 8 | 17819576 | 17819581 | p1.(A)6 |
| 8 | 17819646 | 17819650 | p1.(T)5 |
| 8 | 17820599 | 17820606 | p1.(A)8 |
| 8 | 17822270 | 17822274 | p1.(A)5 |
| 8 | 17823526 | 17823530 | p1.(T)5 |
| 8 | 17824539 | 17824543 | p1.(A)5 |
| 8 | 17827126 | 17827130 | p1.(T)5 |
| 8 | 17827189 | 17827193 | p1.(A)5 |
| 8 | 17842969 | 17842973 | p1.(T)5 |
| 8 | 17849217 | 17849221 | p1.(T)5 |
| 8 | 17868733 | 17868737 | p1.(T)5 |
| 8 | 17868806 | 17868810 | p1.(A)5 |
| 8 | 30916688 | 30916692 | p1.(T)5 |
| 8 | 30938371 | 30938380 | p1.(T)10 |
| 8 | 30938519 | 30938523 | p1.(G)5 |
| 8 | 30958354 | 30958360 | p1.(T)7 |
| 8 | 30958410 | 30958414 | p1.(G)5 |
| 8 | 30969265 | 30969269 | p1.(A)5 |
| 8 | 30973859 | 30973863 | p1.(T)5 |
| 8 | 30973918 | 30973922 | p1.(A)5 |
| 8 | 30982106 | 30982110 | p1.(A)5 |
| 8 | 30989870 | 30989874 | p1.(T)5 |
| 8 | 30989979 | 30989983 | p1.(A)5 |
| 8 | 30999029 | 30999033 | p1.(T)5 |
| 8 | 30999111 | 30999115 | p1.(A)5 |
| 8 | 30999297 | 30999301 | p1.(T)5 |
| 8 | 31001132 | 31001138 | p1.(A)7 |
| 8 | 31004868 | 31004873 | p1.(T)6 |
| 8 | 31004940 | 31004944 | p1.(C)5 |
| 8 | 31007878 | 31007882 | p1.(A)5 |
| 8 | 31014961 | 31014965 | p1.(C)5 |
| 8 | 31030608 | 31030612 | p1.(T)5 |
| 8 | 37553456 | 37553460 | p1.(C)5 |
| 8 | 37553471 | 37553477 | p1.(C)7 |
| 8 | 37553556 | 37553567 | p3.(GCG)4 |
| 8 | 37553617 | 37553621 | p1.(C)5 |
| 8 | 37554758 | 37554769 | p3.(GGC)4 |
| 8 | 37554848 | 37554852 | p1.(G)5 |
| 8 | 37555026 | 37555030 | p1.(C)5 |
| 8 | 37555283 | 37555287 | p1.(G)5 |
| 8 | 37555461 | 37555465 | p1.(G)5 |
| 8 | 37555488 | 37555493 | p1.(C)6 |
| 8 | 37555652 | 37555657 | p1.(G)6 |
| 8 | 37556087 | 37556092 | p1.(G)6 |
| 8 | 37654927 | 37654931 | p1.(G)5 |
| 8 | 37686460 | 37686464 | p1.(G)5 |
| 8 | 37688229 | 37688233 | p1.(G)5 |
| 8 | 37688289 | 37688293 | p1.(G)5 |
| 8 | 37690520 | 37690525 | p1.(C)6 |
| 8 | 37690604 | 37690608 | p1.(G)5 |
| 8 | 37691326 | 37691330 | p1.(T)5 |
| 8 | 37691477 | 37691482 | p1.(C)6 |
| 8 | 37691605 | 37691611 | p1.(G)7 |
| 8 | 37691622 | 37691626 | p1.(C)5 |
| 8 | 37691652 | 37691656 | p1.(G)5 |
| 8 | 37692771 | 37692782 | p4.(GGAG)3 |
| 8 | 37692820 | 37692825 | p1.(C)6 |
| 8 | 37692839 | 37692843 | p1.(C)5 |
| 8 | 37693064 | 37693069 | p1.(C)6 |
| 8 | 37693102 | 37693106 | p1.(C)5 |
| 8 | 37693141 | 37693145 | p1.(C)5 |
| 8 | 37693150 | 37693154 | p1.(C)5 |
| 8 | 37693265 | 37693269 | p1.(C)5 |
| 8 | 37695362 | 37695366 | p1.(G)5 |
| 8 | 37696500 | 37696505 | p1.(G)6 |
| 8 | 37697098 | 37697103 | p1.(G)6 |
| 8 | 37697732 | 37697736 | p1.(C)5 |
| 8 | 37698805 | 37698809 | p1.(C)5 |
| 8 | 37698869 | 37698873 | p1.(C)5 |
| 8 | 37699108 | 37699113 | p1.(C)6 |
| 8 | 37699128 | 37699133 | p1.(C)6 |
| 8 | 37699472 | 37699476 | p1.(G)5 |
| 8 | 37699483 | 37699487 | p1.(G)5 |
| 8 | 37699770 | 37699774 | p1.(C)5 |
| 8 | 37699777 | 37699782 | p1.(G)6 |
| 8 | 38178597 | 38178601 | p1.(T)5 |
| 8 | 38184339 | 38184343 | p1.(C)5 |
| 8 | 38186882 | 38186887 | p1.(A)6 |
| 8 | 38187222 | 38187228 | p1.(T)7 |
| 8 | 38187254 | 38187259 | p1.(T)6 |
| 8 | 38187416 | 38187421 | p1.(A)6 |
| 8 | 38189039 | 38189043 | p1.(T)5 |
| 8 | 38189075 | 38189080 | p1.(A)6 |
| 8 | 38189112 | 38189116 | p1.(A)5 |
| 8 | 38196090 | 38196094 | p1.(T)5 |
| 8 | 38205092 | 38205098 | p1.(T)7 |
| 8 | 38205221 | 38205225 | p1.(T)5 |
| 8 | 38205309 | 38205313 | p1.(T)5 |
| 8 | 38271281 | 38271285 | p1.(G)5 |
| 8 | 38272307 | 38272311 | p1.(T)5 |
| 8 | 38273506 | 38273510 | p1.(G)5 |
| 8 | 38274839 | 38274843 | p1.(C)5 |
| 8 | 38275515 | 38275519 | p1.(G)5 |
| 8 | 38285482 | 38285486 | p1.(T)5 |
| 8 | 38285914 | 38285931 | p3.(TCA)6 |
| 8 | 41789699 | 41789703 | p1.(T)5 |
| 8 | 41790037 | 41790041 | p1.(C)5 |
| 8 | 41790297 | 41790301 | p1.(G)5 |
| 8 | 41790636 | 41790640 | p1.(G)5 |
| 8 | 41790654 | 41790658 | p1.(G)5 |
| 8 | 41790660 | 41790664 | p1.(G)5 |
| 8 | 41790747 | 41790758 | p3.(GGC)4 |
| 8 | 41790787 | 41790798 | p3.(GCT)4 |
| 8 | 41791683 | 41791688 | p1.(A)6 |
| 8 | 41791749 | 41791753 | p1.(T)5 |
| 8 | 41792054 | 41792065 | p3.(CTC)4 |
| 8 | 41792289 | 41792293 | p1.(T)5 |
| 8 | 41792295 | 41792299 | p1.(T)5 |
| 8 | 41798420 | 41798434 | p3.(CTC)5 |
| 8 | 41798569 | 41798573 | p1.(C)5 |
| 8 | 41798776 | 41798780 | p1.(T)5 |
| 8 | 41798792 | 41798796 | p1.(C)5 |
| 8 | 41800494 | 41800498 | p1.(T)5 |
| 8 | 41805325 | 41805330 | p1.(A)6 |
| 8 | 41812907 | 41812911 | p1.(G)5 |
| 8 | 41832291 | 41832295 | p1.(A)5 |
| 8 | 41832349 | 41832355 | p1.(A)7 |
| 8 | 41834604 | 41834609 | p1.(C)6 |
| 8 | 41834638 | 41834643 | p1.(A)6 |
| 8 | 41834752 | 41834763 | p3.(TGA)4 |
| 8 | 41834787 | 41834791 | p1.(T)5 |
| 8 | 41836184 | 41836189 | p1.(T)6 |
| 8 | 41836258 | 41836263 | p1.(T)6 |
| 8 | 41836300 | 41836306 | p1.(A)7 |
| 8 | 41838453 | 41838458 | p1.(A)6 |
| 8 | 41839361 | 41839365 | p1.(T)5 |
| 8 | 41845090 | 41845094 | p1.(A)5 |
| 8 | 41906122 | 41906127 | p1.(A)6 |
| 8 | 41906431 | 41906435 | p1.(T)5 |
| 8 | 41906439 | 41906444 | p1.(T)6 |
| 8 | 42752338 | 42752342 | p1.(G)5 |
| 8 | 42761301 | 42761307 | p1.(T)7 |
| 8 | 42780781 | 42780785 | p1.(T)5 |
| 8 | 42821759 | 42821763 | p1.(A)5 |
| 8 | 42823205 | 42823209 | p1.(A)5 |
| 8 | 42829340 | 42829344 | p1.(A)5 |
| 8 | 42841866 | 42841871 | p1.(A)6 |
| 8 | 42873516 | 42873520 | p1.(A)5 |
| 8 | 48686943 | 48686950 | p1.(A)8 |
| 8 | 48689394 | 48689398 | p1.(A)5 |
| 8 | 48689466 | 48689470 | p1.(G)5 |
| 8 | 48689477 | 48689483 | p1.(T)7 |
| 8 | 48689518 | 48689523 | p1.(T)6 |
| 8 | 48691608 | 48691612 | p1.(T)5 |
| 8 | 48695164 | 48695168 | p1.(A)5 |
| 8 | 48697751 | 48697755 | p1.(G)5 |
| 8 | 48697773 | 48697777 | p1.(T)5 |
| 8 | 48713514 | 48713518 | p1.(T)5 |
| 8 | 48715987 | 48715991 | p1.(T)5 |
| 8 | 48730125 | 48730129 | p1.(A)5 |
| 8 | 48734202 | 48734207 | p1.(G)6 |
| 8 | 48734296 | 48734300 | p1.(A)5 |
| 8 | 48736512 | 48736516 | p1.(A)5 |
| 8 | 48743156 | 48743160 | p1.(A)5 |
| 8 | 48743276 | 48743280 | p1.(T)5 |
| 8 | 48744384 | 48744388 | p1.(T)5 |
| 8 | 48746799 | 48746805 | p1.(T)7 |
| 8 | 48749870 | 48749874 | p1.(A)5 |
| 8 | 48752732 | 48752736 | p1.(T)5 |
| 8 | 48762068 | 48762072 | p1.(A)5 |
| 8 | 48765235 | 48765240 | p1.(T)6 |
| 8 | 48767862 | 48767866 | p1.(T)5 |
| 8 | 48767931 | 48767935 | p1.(C)5 |
| 8 | 48773510 | 48773514 | p1.(T)5 |
| 8 | 48773512 | 48773523 | p4.(TTTC)3 |
| 8 | 48774943 | 48774947 | p1.(T)5 |
| 8 | 48776024 | 48776028 | p1.(T)5 |
| 8 | 48776143 | 48776147 | p1.(A)5 |
| 8 | 48777257 | 48777261 | p1.(G)5 |
| 8 | 48790417 | 48790422 | p1.(A)6 |
| 8 | 48792053 | 48792057 | p1.(T)5 |
| 8 | 48794481 | 48794485 | p1.(T)5 |
| 8 | 48798560 | 48798564 | p1.(T)5 |
| 8 | 48798714 | 48798718 | p1.(A)5 |
| 8 | 48800098 | 48800102 | p1.(T)5 |
| 8 | 48805809 | 48805813 | p1.(C)5 |
| 8 | 48805866 | 48805871 | p1.(C)6 |
| 8 | 48805952 | 48805956 | p1.(A)5 |
| 8 | 48815313 | 48815317 | p1.(A)5 |
| 8 | 48825049 | 48825053 | p1.(C)5 |
| 8 | 48827899 | 48827903 | p1.(T)5 |
| 8 | 48839881 | 48839885 | p1.(G)5 |
| 8 | 48841643 | 48841647 | p1.(A)5 |
| 8 | 48842539 | 48842544 | p1.(A)6 |
| 8 | 48842577 | 48842581 | p1.(A)5 |
| 8 | 48843263 | 48843267 | p1.(A)5 |
| 8 | 48845567 | 48845571 | p1.(A)5 |
| 8 | 48845704 | 48845708 | p1.(A)5 |
| 8 | 48848382 | 48848386 | p1.(T)5 |
| 8 | 48852117 | 48852121 | p1.(A)5 |
| 8 | 48852217 | 48852221 | p1.(T)5 |
| 8 | 48852235 | 48852239 | p1.(T)5 |
| 8 | 48855805 | 48855810 | p1.(T)6 |
| 8 | 54900673 | 54900677 | p1.(C)5 |
| 8 | 54912512 | 54912517 | p1.(T)6 |
| 8 | 57078932 | 57078937 | p1.(G)6 |
| 8 | 57079050 | 57079054 | p1.(A)5 |
| 8 | 57079074 | 57079078 | p1.(G)5 |
| 8 | 57079242 | 57079246 | p1.(C)5 |
| 8 | 57079261 | 57079265 | p1.(T)5 |
| 8 | 57079754 | 57079760 | p1.(T)7 |
| 8 | 57080731 | 57080736 | p1.(T)6 |
| 8 | 57129070 | 57129074 | p1.(A)5 |
| 8 | 71036967 | 71036971 | p1.(G)5 |
| 8 | 71037104 | 71037114 | p1.(A)11 |
| 8 | 71050537 | 71050542 | p1.(C)6 |
| 8 | 71068308 | 71068312 | p1.(G)5 |
| 8 | 71068744 | 71068748 | p1.(G)5 |
| 8 | 71068882 | 71068886 | p1.(G)5 |
| 8 | 71068925 | 71068929 | p1.(T)5 |
| 8 | 71069263 | 71069267 | p1.(C)5 |
| 8 | 71069457 | 71069466 | p2.(CA)5 |
| 8 | 71071846 | 71071850 | p1.(A)5 |
| 8 | 71078980 | 71078984 | p1.(C)5 |
| 8 | 71082457 | 71082461 | p1.(T)5 |
| 8 | 71126235 | 71126239 | p1.(A)5 |
| 8 | 71126289 | 71126293 | p1.(T)5 |
| 8 | 80677425 | 80677429 | p1.(A)5 |
| 8 | 80677444 | 80677448 | p1.(C)5 |
| 8 | 80677640 | 80677644 | p1.(G)5 |
| 8 | 80677795 | 80677799 | p1.(C)5 |
| 8 | 80678947 | 80678951 | p1.(T)5 |
| 8 | 80679465 | 80679469 | p1.(T)5 |
| 8 | 80679910 | 80679914 | p1.(G)5 |
| 8 | 90947779 | 90947783 | p1.(T)5 |
| 8 | 90947794 | 90947798 | p1.(T)5 |
| 8 | 90947821 | 90947825 | p1.(T)5 |
| 8 | 90958369 | 90958373 | p1.(T)5 |
| 8 | 90958387 | 90958392 | p1.(T)6 |
| 8 | 90958441 | 90958445 | p1.(T)5 |
| 8 | 90958480 | 90958485 | p1.(T)6 |
| 8 | 90965543 | 90965547 | p1.(T)5 |
| 8 | 90965571 | 90965575 | p1.(T)5 |
| 8 | 90965666 | 90965672 | p1.(T)7 |
| 8 | 90965716 | 90965720 | p1.(T)5 |
| 8 | 90965809 | 90965813 | p1.(T)5 |
| 8 | 90967501 | 90967505 | p1.(A)5 |
| 8 | 90967512 | 90967518 | p1.(T)7 |
| 8 | 90967555 | 90967566 | p3.(CTG)4 |
| 8 | 90967732 | 90967736 | p1.(T)5 |
| 8 | 90967790 | 90967794 | p1.(A)5 |
| 8 | 90971024 | 90971028 | p1.(T)5 |
| 8 | 90982697 | 90982701 | p1.(A)5 |
| 8 | 90982788 | 90982792 | p1.(A)5 |
| 8 | 90983414 | 90983418 | p1.(A)5 |
| 8 | 90983477 | 90983481 | p1.(T)5 |
| 8 | 90983514 | 90983518 | p1.(A)5 |
| 8 | 90983523 | 90983527 | p1.(A)5 |
| 8 | 90995032 | 90995036 | p1.(T)5 |
| 8 | 92998419 | 92998425 | p1.(T)7 |
| 8 | 92998520 | 92998525 | p1.(T)6 |
| 8 | 92999205 | 92999209 | p1.(A)5 |
| 8 | 93004123 | 93004133 | p1.(G)5(A)6 |
| 8 | 93026928 | 93026932 | p1.(G)5 |
| 8 | 93027002 | 93027006 | p1.(G)5 |
| 8 | 93029501 | 93029505 | p1.(G)5 |
| 8 | 100899849 | 100899853 | p1.(A)5 |
| 8 | 100904153 | 100904157 | p1.(C)5 |
| 8 | 103266520 | 103266524 | p1.(T)5 |
| 8 | 103271320 | 103271325 | p1.(T)6 |
| 8 | 103273493 | 103273497 | p1.(A)5 |
| 8 | 103274272 | 103274278 | p1.(A)7 |
| 8 | 103274301 | 103274306 | p1.(A)6 |
| 8 | 103276789 | 103276793 | p1.(A)5 |
| 8 | 103283437 | 103283446 | p2.(TC)5 |
| 8 | 103284806 | 103284810 | p1.(T)5 |
| 8 | 103284819 | 103284823 | p1.(A)5 |
| 8 | 103284844 | 103284848 | p1.(A)5 |
| 8 | 103289261 | 103289265 | p1.(T)5 |
| 8 | 103289349 | 103289356 | p1.(T)8 |
| 8 | 103289380 | 103289384 | p1.(T)5 |
| 8 | 103289405 | 103289409 | p1.(T)5 |
| 8 | 103291141 | 103291145 | p1.(A)5 |
| 8 | 103291328 | 103291332 | p1.(G)5 |
| 8 | 103291370 | 103291374 | p1.(A)5 |
| 8 | 103291445 | 103291449 | p1.(A)5 |
| 8 | 103292700 | 103292704 | p1.(A)5 |
| 8 | 103293591 | 103293595 | p1.(A)5 |
| 8 | 103298018 | 103298022 | p1.(A)5 |
| 8 | 103298852 | 103298856 | p1.(T)5 |
| 8 | 103299795 | 103299800 | p1.(A)6 |
| 8 | 103306080 | 103306084 | p1.(A)5 |
| 8 | 103306183 | 103306187 | p1.(T)5 |
| 8 | 103306189 | 103306193 | p1.(T)5 |
| 8 | 103306224 | 103306228 | p1.(A)5 |
| 8 | 103306348 | 103306353 | p1.(A)6 |
| 8 | 103307567 | 103307571 | p1.(A)5 |
| 8 | 103307657 | 103307661 | p1.(T)5 |
| 8 | 103309812 | 103309816 | p1.(T)5 |
| 8 | 103309837 | 103309842 | p1.(A)6 |
| 8 | 103311747 | 103311751 | p1.(G)5 |
| 8 | 103312263 | 103312267 | p1.(G)5 |
| 8 | 103312274 | 103312279 | p1.(G)6 |
| 8 | 103323558 | 103323562 | p1.(T)5 |
| 8 | 103324395 | 103324400 | p1.(T)6 |
| 8 | 103324415 | 103324419 | p1.(T)5 |
| 8 | 103326065 | 103326069 | p1.(A)5 |
| 8 | 103335562 | 103335566 | p1.(T)5 |
| 8 | 103335572 | 103335576 | p1.(T)5 |
| 8 | 103338829 | 103338833 | p1.(T)5 |
| 8 | 103338835 | 103338840 | p1.(T)6 |
| 8 | 103338900 | 103338905 | p1.(A)6 |
| 8 | 103341394 | 103341398 | p1.(A)5 |
| 8 | 103354687 | 103354692 | p1.(T)6 |
| 8 | 103358631 | 103358636 | p1.(A)6 |
| 8 | 103372393 | 103372397 | p1.(C)5 |
| 8 | 103372421 | 103372425 | p1.(G)5 |
| 8 | 103424497 | 103424501 | p1.(C)5 |
| 8 | 103424517 | 103424522 | p1.(C)6 |
| 8 | 117859821 | 117859825 | p1.(T)5 |
| 8 | 117861249 | 117861253 | p1.(C)5 |
| 8 | 117861275 | 117861279 | p1.(A)5 |
| 8 | 117862878 | 117862882 | p1.(T)5 |
| 8 | 117862902 | 117862906 | p1.(T)5 |
| 8 | 117862960 | 117862964 | p1.(G)5 |
| 8 | 117864899 | 117864903 | p1.(T)5 |
| 8 | 117864953 | 117864966 | p1.(A)14 |
| 8 | 117866532 | 117866536 | p1.(T)5 |
| 8 | 117868962 | 117868966 | p1.(G)5 |
| 8 | 118811931 | 118811935 | p1.(C)5 |
| 8 | 118817137 | 118817141 | p1.(A)5 |
| 8 | 118831983 | 118831988 | p1.(G)6 |
| 8 | 118832020 | 118832025 | p1.(G)6 |
| 8 | 118832041 | 118832045 | p1.(A)5 |
| 8 | 118842504 | 118842508 | p1.(A)5 |
| 8 | 118847797 | 118847802 | p1.(A)6 |
| 8 | 119122534 | 119122543 | p1.(A)5(C)5 |
| 8 | 119122707 | 119122711 | p1.(A)5 |
| 8 | 119122917 | 119122921 | p1.(T)5 |
| 8 | 119122951 | 119122955 | p1.(T)5 |
| 8 | 119123039 | 119123044 | p1.(G)6 |
| 8 | 119123271 | 119123276 | p1.(T)6 |
| 8 | 128750605 | 128750619 | p3.(CAG)5 |
| 8 | 128750632 | 128750636 | p1.(C)5 |
| 8 | 128750884 | 128750888 | p1.(A)5 |
| 8 | 128753004 | 128753009 | p1.(T)6 |
| 8 | 128753053 | 128753057 | p1.(C)5 |
| 8 | 128753073 | 128753078 | p1.(A)6 |
| 8 | 134251178 | 134251182 | p1.(G)5 |
| 8 | 134251198 | 134251202 | p1.(C)5 |
| 8 | 134266788 | 134266792 | p1.(A)5 |
| 8 | 134269008 | 134269012 | p1.(C)5 |
| 8 | 134270678 | 134270682 | p1.(A)5 |
| 8 | 134274390 | 134274394 | p1.(G)5 |
| 8 | 145736797 | 145736801 | p1.(C)5 |
| 8 | 145737639 | 145737643 | p1.(C)5 |
| 8 | 145737936 | 145737940 | p1.(G)5 |
| 8 | 145738055 | 145738059 | p1.(C)5 |
| 8 | 145738157 | 145738161 | p1.(G)5 |
| 8 | 145738349 | 145738354 | p1.(G)6 |
| 8 | 145738582 | 145738593 | p1.(G)12 |
| 8 | 145738686 | 145738690 | p1.(G)5 |
| 8 | 145738854 | 145738858 | p1.(G)5 |
| 8 | 145739692 | 145739696 | p1.(C)5 |
| 8 | 145739819 | 145739823 | p1.(C)5 |
| 8 | 145741231 | 145741235 | p1.(C)5 |
| 8 | 145741277 | 145741282 | p1.(G)6 |
| 8 | 145741488 | 145741493 | p1.(G)6 |
| 8 | 145741638 | 145741642 | p1.(C)5 |
| 8 | 145741649 | 145741653 | p1.(G)5 |
| 8 | 145741746 | 145741750 | p1.(G)5 |
| 8 | 145741888 | 145741897 | p1.(G)5(C)5 |
| 8 | 145742041 | 145742045 | p1.(T)5 |
| 8 | 145742075 | 145742079 | p1.(G)5 |
| 8 | 145742551 | 145742555 | p1.(C)5 |
| 8 | 145743170 | 145743179 | p2.(GC)5 |
| 9 | 5021975 | 5021979 | p1.(A)5 |
| 9 | 5050673 | 5050677 | p1.(T)5 |
| 9 | 5054549 | 5054554 | p1.(T)6 |
| 9 | 5054799 | 5054803 | p1.(T)5 |
| 9 | 5055780 | 5055784 | p1.(A)5 |
| 9 | 5066768 | 5066773 | p1.(T)6 |
| 9 | 5069060 | 5069065 | p1.(A)6 |
| 9 | 5069193 | 5069197 | p1.(C)5 |
| 9 | 5069916 | 5069921 | p1.(T)6 |
| 9 | 5072526 | 5072530 | p1.(T)5 |
| 9 | 5073682 | 5073691 | p1.(T)10 |
| 9 | 5078342 | 5078346 | p1.(A)5 |
| 9 | 5080279 | 5080283 | p1.(A)5 |
| 9 | 5089745 | 5089750 | p1.(A)6 |
| 9 | 5090509 | 5090513 | p1.(A)5 |
| 9 | 5090759 | 5090763 | p1.(A)5 |
| 9 | 5126454 | 5126462 | p1.(T)9 |
| 9 | 5457072 | 5457076 | p1.(T)5 |
| 9 | 5457160 | 5457164 | p1.(A)5 |
| 9 | 5462974 | 5462985 | p3.(ACC)4 |
| 9 | 5465480 | 5465494 | p5.(GTTTT)3 |
| 9 | 5465491 | 5465495 | p1.(T)5 |
| 9 | 5549526 | 5549530 | p1.(C)5 |
| 9 | 5563169 | 5563173 | p1.(A)5 |
| 9 | 14120432 | 14120436 | p1.(T)5 |
| 9 | 14125670 | 14125674 | p1.(G)5 |
| 9 | 14150181 | 14150185 | p1.(C)5 |
| 9 | 14307011 | 14307015 | p1.(A)5 |
| 9 | 14307419 | 14307423 | p1.(T)5 |
| 9 | 15472612 | 15472622 | p1.(A)11 |
| 9 | 15474077 | 15474081 | p1.(T)5 |
| 9 | 15474084 | 15474088 | p1.(T)5 |
| 9 | 15474112 | 15474117 | p1.(T)6 |
| 9 | 15474179 | 15474183 | p1.(T)5 |
| 9 | 15474190 | 15474194 | p1.(T)5 |
| 9 | 15474206 | 15474210 | p1.(T)5 |
| 9 | 15479677 | 15479681 | p1.(T)5 |
| 9 | 15486837 | 15486841 | p1.(T)5 |
| 9 | 15490114 | 15490119 | p1.(A)6 |
| 9 | 15490125 | 15490130 | p1.(A)6 |
| 9 | 15510192 | 15510196 | p1.(G)5 |
| 9 | 15510204 | 15510208 | p1.(G)5 |
| 9 | 20346378 | 20346391 | p1.(A)14 |
| 9 | 20346394 | 20346405 | p1.(A)12 |
| 9 | 20360754 | 20360758 | p1.(T)5 |
| 9 | 20413869 | 20413874 | p1.(T)6 |
| 9 | 20413913 | 20413917 | p1.(A)5 |
| 9 | 20413945 | 20413949 | p1.(T)5 |
| 9 | 20413952 | 20413958 | p1.(T)7 |
| 9 | 20414226 | 20414230 | p1.(T)5 |
| 9 | 20414264 | 20414268 | p1.(A)5 |
| 9 | 20414278 | 20414295 | p3.(CTG)6 |
| 9 | 20414311 | 20414340 | p3.(CTG)10 |
| 9 | 20414344 | 20414373 | p3.(CTG)10 |
| 9 | 20620656 | 20620660 | p1.(T)5 |
| 9 | 20620780 | 20620785 | p1.(T)6 |
| 9 | 20622260 | 20622264 | p1.(G)5 |
| 9 | 20622295 | 20622302 | p1.(C)8 |
| 9 | 20622310 | 20622316 | p1.(C)7 |
| 9 | 21970951 | 21970956 | p1.(C)6 |
| 9 | 21971164 | 21971175 | p3.(AGC)4 |
| 9 | 21974721 | 21974725 | p1.(C)5 |
| 9 | 22005991 | 22005995 | p1.(C)5 |
| 9 | 22006203 | 22006214 | p3.(AGC)4 |
| 9 | 22008918 | 22008922 | p1.(C)5 |
| 9 | 22008960 | 22008964 | p1.(C)5 |
| 9 | 35074203 | 35074207 | p1.(G)5 |
| 9 | 35075283 | 35075287 | p1.(T)5 |
| 9 | 35075485 | 35075489 | p1.(T)5 |
| 9 | 35075702 | 35075706 | p1.(A)5 |
| 9 | 35075737 | 35075742 | p1.(G)6 |
| 9 | 35076762 | 35076766 | p1.(C)5 |
| 9 | 35077347 | 35077351 | p1.(G)5 |
| 9 | 36840626 | 36840630 | p1.(G)5 |
| 9 | 36882050 | 36882056 | p1.(G)7 |
| 9 | 37002679 | 37002683 | p1.(G)5 |
| 9 | 37015109 | 37015114 | p1.(T)6 |
| 9 | 37020764 | 37020775 | p1.(A)5(C)7 |
| 9 | 80343588 | 80343601 | p1.(A)14 |
| 9 | 80409491 | 80409495 | p1.(C)5 |
| 9 | 87317312 | 87317316 | p1.(A)5 |
| 9 | 87325715 | 87325719 | p1.(T)5 |
| 9 | 87342556 | 87342560 | p1.(T)5 |
| 9 | 87563493 | 87563497 | p1.(C)5 |
| 9 | 87635213 | 87635217 | p1.(G)5 |
| 9 | 87636217 | 87636221 | p1.(C)5 |
| 9 | 93606226 | 93606230 | p1.(T)5 |
| 9 | 93606273 | 93606278 | p1.(G)6 |
| 9 | 93607789 | 93607794 | p1.(A)6 |
| 9 | 93607811 | 93607815 | p1.(A)5 |
| 9 | 93626932 | 93626937 | p1.(A)6 |
| 9 | 93636955 | 93636959 | p1.(C)5 |
| 9 | 93637106 | 93637110 | p1.(A)5 |
| 9 | 93637127 | 93637131 | p1.(A)5 |
| 9 | 93639884 | 93639888 | p1.(A)5 |
| 9 | 95177534 | 95177545 | p3.(TCA)4 |
| 9 | 95179010 | 95179014 | p1.(A)5 |
| 9 | 95179088 | 95179092 | p1.(T)5 |
| 9 | 95179202 | 95179206 | p1.(T)5 |
| 9 | 95179799 | 95179804 | p1.(A)6 |
| 9 | 95179830 | 95179835 | p1.(A)6 |
| 9 | 95179845 | 95179855 | p1.(T)11 |
| 9 | 97864031 | 97864035 | p1.(T)5 |
| 9 | 97873777 | 97873781 | p1.(G)5 |
| 9 | 97873817 | 97873822 | p1.(G)6 |
| 9 | 97887369 | 97887380 | p4.(TGCT)3 |
| 9 | 97897697 | 97897701 | p1.(A)5 |
| 9 | 97897715 | 97897719 | p1.(T)5 |
| 9 | 97912296 | 97912300 | p1.(G)5 |
| 9 | 98011579 | 98011583 | p1.(A)5 |
| 9 | 98209301 | 98209305 | p1.(G)5 |
| 9 | 98209351 | 98209355 | p1.(C)5 |
| 9 | 98209358 | 98209362 | p1.(G)5 |
| 9 | 98209593 | 98209598 | p1.(G)6 |
| 9 | 98209617 | 98209623 | p1.(G)7 |
| 9 | 98211549 | 98211554 | p1.(G)6 |
| 9 | 98220484 | 98220488 | p1.(T)5 |
| 9 | 98222070 | 98222074 | p1.(A)5 |
| 9 | 98229671 | 98229675 | p1.(C)5 |
| 9 | 98229687 | 98229691 | p1.(A)5 |
| 9 | 98231105 | 98231110 | p1.(G)6 |
| 9 | 98231272 | 98231276 | p1.(G)5 |
| 9 | 98231358 | 98231363 | p1.(G)6 |
| 9 | 98232166 | 98232170 | p1.(A)5 |
| 9 | 98238450 | 98238456 | p1.(A)7 |
| 9 | 98239029 | 98239033 | p1.(T)5 |
| 9 | 98239095 | 98239099 | p1.(A)5 |
| 9 | 98239147 | 98239151 | p1.(A)5 |
| 9 | 98242380 | 98242384 | p1.(A)5 |
| 9 | 98242682 | 98242686 | p1.(T)5 |
| 9 | 98242690 | 98242694 | p1.(G)5 |
| 9 | 98268793 | 98268799 | p1.(T)7 |
| 9 | 98270530 | 98270536 | p1.(C)7 |
| 9 | 98270593 | 98270604 | p3.(GCC)4 |
| 9 | 98270647 | 98270667 | p3.(GCC)7 |
| 9 | 100437715 | 100437720 | p1.(A)6 |
| 9 | 100437726 | 100437731 | p1.(T)6 |
| 9 | 100437873 | 100437881 | p1.(A)9 |
| 9 | 100447212 | 100447217 | p1.(T)6 |
| 9 | 100447238 | 100447243 | p1.(T)6 |
| 9 | 100449461 | 100449466 | p1.(T)6 |
| 9 | 100455962 | 100455976 | p3.(TTC)5 |
| 9 | 102590616 | 102590642 | p3.(CAC)9 |
| 9 | 102590749 | 102590753 | p1.(C)5 |
| 9 | 102590768 | 102590773 | p1.(C)6 |
| 9 | 102590780 | 102590784 | p1.(G)5 |
| 9 | 102590922 | 102590927 | p1.(C)6 |
| 9 | 102591267 | 102591271 | p1.(T)5 |
| 9 | 102594983 | 102594987 | p1.(A)5 |
| 9 | 102595013 | 102595017 | p1.(A)5 |
| 9 | 102607096 | 102607100 | p1.(T)5 |
| 9 | 108424883 | 108424887 | p1.(A)5 |
| 9 | 108424895 | 108424899 | p1.(A)5 |
| 9 | 108424951 | 108424955 | p1.(G)5 |
| 9 | 110247979 | 110247984 | p1.(A)6 |
| 9 | 110248034 | 110248038 | p1.(A)5 |
| 9 | 110248092 | 110248096 | p1.(T)5 |
| 9 | 110249301 | 110249305 | p1.(C)5 |
| 9 | 110249756 | 110249760 | p1.(G)5 |
| 9 | 110249797 | 110249801 | p1.(G)5 |
| 9 | 110250136 | 110250140 | p1.(G)5 |
| 9 | 110250508 | 110250512 | p1.(G)5 |
| 9 | 110251460 | 110251464 | p1.(G)5 |
| 9 | 123850624 | 123850629 | p1.(A)6 |
| 9 | 123850829 | 123850833 | p1.(T)5 |
| 9 | 123852600 | 123852605 | p1.(A)6 |
| 9 | 123857182 | 123857186 | p1.(A)5 |
| 9 | 123875985 | 123875993 | p1.(A)9 |
| 9 | 123876006 | 123876012 | p1.(A)7 |
| 9 | 123886191 | 123886201 | p1.(T)11 |
| 9 | 123888221 | 123888227 | p1.(T)7 |
| 9 | 123898238 | 123898242 | p1.(A)5 |
| 9 | 123902907 | 123902911 | p1.(C)5 |
| 9 | 123903071 | 123903075 | p1.(A)5 |
| 9 | 123904446 | 123904450 | p1.(A)5 |
| 9 | 123904543 | 123904547 | p1.(A)5 |
| 9 | 123907228 | 123907232 | p1.(G)5 |
| 9 | 123907509 | 123907515 | p1.(T)7 |
| 9 | 123908514 | 123908525 | p3.(CCA)4 |
| 9 | 123911070 | 123911074 | p1.(C)5 |
| 9 | 123912504 | 123912508 | p1.(C)5 |
| 9 | 123912667 | 123912671 | p1.(C)5 |
| 9 | 123912690 | 123912695 | p1.(C)6 |
| 9 | 123916991 | 123916996 | p1.(T)6 |
| 9 | 123917049 | 123917053 | p1.(A)5 |
| 9 | 123919750 | 123919755 | p1.(A)6 |
| 9 | 123920143 | 123920147 | p1.(A)5 |
| 9 | 123921184 | 123921188 | p1.(A)5 |
| 9 | 123921303 | 123921308 | p1.(T)6 |
| 9 | 123924525 | 123924531 | p1.(A)7 |
| 9 | 123928385 | 123928389 | p1.(A)5 |
| 9 | 123929885 | 123929896 | p3.(AAC)4 |
| 9 | 123932010 | 123932021 | p4.(AGAA)3 |
| 9 | 123932083 | 123932087 | p1.(A)5 |
| 9 | 123933690 | 123933694 | p1.(A)5 |
| 9 | 123935531 | 123935535 | p1.(A)5 |
| 9 | 123935756 | 123935760 | p1.(A)5 |
| 9 | 131454204 | 131454209 | p1.(T)6 |
| 9 | 131454245 | 131454249 | p1.(T)5 |
| 9 | 132658125 | 132658129 | p1.(T)5 |
| 9 | 132658279 | 132658283 | p1.(A)5 |
| 9 | 132665264 | 132665268 | p1.(T)5 |
| 9 | 132671226 | 132671230 | p1.(T)5 |
| 9 | 132686221 | 132686225 | p1.(G)5 |
| 9 | 132687243 | 132687250 | p1.(T)8 |
| 9 | 132689475 | 132689479 | p1.(T)5 |
| 9 | 132691983 | 132691987 | p1.(A)5 |
| 9 | 132757214 | 132757218 | p1.(T)5 |
| 9 | 133730247 | 133730251 | p1.(A)5 |
| 9 | 133738286 | 133738290 | p1.(C)5 |
| 9 | 133738348 | 133738352 | p1.(G)5 |
| 9 | 133748264 | 133748268 | p1.(C)5 |
| 9 | 133748408 | 133748412 | p1.(A)5 |
| 9 | 133756058 | 133756062 | p1.(C)5 |
| 9 | 133759412 | 133759416 | p1.(C)5 |
| 9 | 133759460 | 133759464 | p1.(A)5 |
| 9 | 133759490 | 133759504 | p3.(AAG)5 |
| 9 | 133759623 | 133759627 | p1.(C)5 |
| 9 | 133759706 | 133759710 | p1.(G)5 |
| 9 | 133760024 | 133760029 | p1.(C)6 |
| 9 | 133760039 | 133760043 | p1.(A)5 |
| 9 | 133760167 | 133760171 | p1.(C)5 |
| 9 | 133760232 | 133760236 | p1.(C)5 |
| 9 | 133760264 | 133760268 | p1.(G)5 |
| 9 | 133760378 | 133760382 | p1.(C)5 |
| 9 | 133760543 | 133760547 | p1.(A)5 |
| 9 | 133760598 | 133760602 | p1.(C)5 |
| 9 | 133760613 | 133760617 | p1.(C)5 |
| 9 | 133760858 | 133760862 | p1.(A)5 |
| 9 | 134003047 | 134003051 | p1.(T)5 |
| 9 | 134003058 | 134003062 | p1.(A)5 |
| 9 | 134003840 | 134003846 | p1.(T)7 |
| 9 | 134004676 | 134004680 | p1.(A)5 |
| 9 | 134007993 | 134008000 | p1.(A)8 |
| 9 | 134010285 | 134010290 | p1.(A)6 |
| 9 | 134015925 | 134015930 | p1.(T)6 |
| 9 | 134019779 | 134019783 | p1.(T)5 |
| 9 | 134019805 | 134019809 | p1.(C)5 |
| 9 | 134019867 | 134019871 | p1.(C)5 |
| 9 | 134019942 | 134019946 | p1.(C)5 |
| 9 | 134019964 | 134019968 | p1.(C)5 |
| 9 | 134021508 | 134021512 | p1.(T)5 |
| 9 | 134021565 | 134021569 | p1.(C)5 |
| 9 | 134022865 | 134022869 | p1.(T)5 |
| 9 | 134027114 | 134027118 | p1.(T)5 |
| 9 | 134034756 | 134034762 | p1.(T)7 |
| 9 | 134034863 | 134034868 | p1.(A)6 |
| 9 | 134039457 | 134039461 | p1.(C)5 |
| 9 | 134049595 | 134049599 | p1.(C)5 |
| 9 | 134062668 | 134062672 | p1.(T)5 |
| 9 | 134072590 | 134072594 | p1.(T)5 |
| 9 | 134072656 | 134072660 | p1.(G)5 |
| 9 | 134072880 | 134072884 | p1.(T)5 |
| 9 | 134073008 | 134073014 | p1.(C)7 |
| 9 | 134073037 | 134073041 | p1.(C)5 |
| 9 | 134073110 | 134073114 | p1.(T)5 |
| 9 | 134073195 | 134073199 | p1.(T)5 |
| 9 | 134073232 | 134073236 | p1.(C)5 |
| 9 | 134073326 | 134073330 | p1.(C)5 |
| 9 | 134073493 | 134073498 | p1.(A)6 |
| 9 | 134073597 | 134073601 | p1.(G)5 |
| 9 | 134073722 | 134073726 | p1.(C)5 |
| 9 | 134073881 | 134073885 | p1.(C)5 |
| 9 | 134074264 | 134074269 | p1.(G)6 |
| 9 | 134090591 | 134090596 | p1.(T)6 |
| 9 | 134090650 | 134090654 | p1.(G)5 |
| 9 | 134090704 | 134090708 | p1.(A)5 |
| 9 | 134090728 | 134090732 | p1.(G)5 |
| 9 | 134103591 | 134103595 | p1.(G)5 |
| 9 | 134103724 | 134103728 | p1.(C)5 |
| 9 | 134106070 | 134106074 | p1.(C)5 |
| 9 | 134107704 | 134107713 | p1.(C)5(T)5 |
| 9 | 135771689 | 135771693 | p1.(G)5 |
| 9 | 135771861 | 135771865 | p1.(T)5 |
| 9 | 135771962 | 135771966 | p1.(G)5 |
| 9 | 135771988 | 135772005 | p3.(GCT)6 |
| 9 | 135772054 | 135772058 | p1.(G)5 |
| 9 | 135772600 | 135772604 | p1.(T)5 |
| 9 | 135772615 | 135772620 | p1.(T)6 |
| 9 | 135772902 | 135772906 | p1.(T)5 |
| 9 | 135772951 | 135772957 | p1.(T)7 |
| 9 | 135773001 | 135773018 | p1.(A)18 |
| 9 | 135776231 | 135776236 | p1.(A)6 |
| 9 | 135781141 | 135781145 | p1.(A)5 |
| 9 | 135781157 | 135781161 | p1.(G)5 |
| 9 | 135782131 | 135782135 | p1.(T)5 |
| 9 | 135782168 | 135782172 | p1.(A)5 |
| 9 | 135785964 | 135785969 | p1.(G)6 |
| 9 | 135785984 | 135785988 | p1.(G)5 |
| 9 | 135787662 | 135787666 | p1.(T)5 |
| 9 | 135797261 | 135797265 | p1.(A)5 |
| 9 | 135798769 | 135798773 | p1.(A)5 |
| 9 | 135804209 | 135804213 | p1.(G)5 |
| 9 | 135973945 | 135973956 | p4.(CCAG)3 |
| 9 | 135975698 | 135975709 | p3.(CTC)4 |
| 9 | 135977929 | 135977933 | p1.(G)5 |
| 9 | 135985013 | 135985017 | p1.(T)5 |
| 9 | 136898775 | 136898786 | p3.(GCT)4 |
| 9 | 136898800 | 136898804 | p1.(C)5 |
| 9 | 136901171 | 136901175 | p1.(T)5 |
| 9 | 136905293 | 136905304 | p3.(CTT)4 |
| 9 | 136906996 | 136907000 | p1.(G)5 |
| 9 | 136907005 | 136907009 | p1.(G)5 |
| 9 | 136910452 | 136910456 | p1.(G)5 |
| 9 | 136913497 | 136913501 | p1.(G)5 |
| 9 | 136913572 | 136913576 | p1.(T)5 |
| 9 | 136915566 | 136915571 | p1.(G)6 |
| 9 | 136915646 | 136915651 | p1.(G)6 |
| 9 | 136915664 | 136915668 | p1.(G)5 |
| 9 | 136916734 | 136916738 | p1.(G)5 |
| 9 | 136916775 | 136916779 | p1.(T)5 |
| 9 | 136916784 | 136916788 | p1.(A)5 |
| 9 | 136917543 | 136917547 | p1.(T)5 |
| 9 | 136918529 | 136918536 | p1.(G)8 |
| 9 | 136918573 | 136918577 | p1.(G)5 |
| 9 | 139390697 | 139390701 | p1.(G)5 |
| 9 | 139390736 | 139390741 | p1.(G)6 |
| 9 | 139390945 | 139390959 | p3.(GTG)5 |
| 9 | 139391171 | 139391175 | p1.(G)5 |
| 9 | 139391188 | 139391192 | p1.(G)5 |
| 9 | 139391207 | 139391211 | p1.(C)5 |
| 9 | 139391409 | 139391413 | p1.(C)5 |
| 9 | 139391778 | 139391782 | p1.(G)5 |
| 9 | 139391799 | 139391804 | p1.(C)6 |
| 9 | 139392013 | 139392017 | p1.(G)5 |
| 9 | 139395260 | 139395264 | p1.(C)5 |
| 9 | 139396258 | 139396262 | p1.(G)5 |
| 9 | 139396482 | 139396487 | p1.(C)6 |
| 9 | 139396543 | 139396548 | p1.(G)6 |
| 9 | 139396919 | 139396923 | p1.(G)5 |
| 9 | 139399409 | 139399420 | p3.(CAC)4 |
| 9 | 139399552 | 139399556 | p1.(G)5 |
| 9 | 139399561 | 139399566 | p1.(G)6 |
| 9 | 139400023 | 139400028 | p1.(G)6 |
| 9 | 139400047 | 139400051 | p1.(C)5 |
| 9 | 139401034 | 139401038 | p1.(C)5 |
| 9 | 139401270 | 139401274 | p1.(C)5 |
| 9 | 139401385 | 139401390 | p1.(G)6 |
| 9 | 139401813 | 139401817 | p1.(C)5 |
| 9 | 139402400 | 139402404 | p1.(C)5 |
| 9 | 139402596 | 139402600 | p1.(G)5 |
| 9 | 139403348 | 139403352 | p1.(G)5 |
| 9 | 139403530 | 139403534 | p1.(G)5 |
| 9 | 139404395 | 139404399 | p1.(C)5 |
| 9 | 139405686 | 139405690 | p1.(G)5 |
| 9 | 139407996 | 139408000 | p1.(G)5 |
| 9 | 139409097 | 139409101 | p1.(C)5 |
| 9 | 139409991 | 139409995 | p1.(C)5 |
| 9 | 139410494 | 139410498 | p1.(G)5 |
| 9 | 139410555 | 139410569 | p5.(GGGGA)3 |
| 9 | 139412303 | 139412307 | p1.(G)5 |
| 9 | 139413951 | 139413955 | p1.(C)5 |
| 9 | 139417304 | 139417315 | p4.(GGCA)3 |
| 9 | 139417352 | 139417356 | p1.(C)5 |
| 9 | 139418228 | 139418232 | p1.(C)5 |
| 9 | 139418371 | 139418375 | p1.(G)5 |
| 10 | 8097767 | 8097771 | p1.(T)5 |
| 10 | 8100355 | 8100359 | p1.(C)5 |
| 10 | 8100425 | 8100430 | p1.(C)6 |
| 10 | 8100452 | 8100457 | p1.(G)6 |
| 10 | 8100728 | 8100734 | p1.(C)7 |
| 10 | 8100754 | 8100758 | p1.(C)5 |
| 10 | 8115752 | 8115756 | p1.(A)5 |
| 10 | 8115771 | 8115775 | p1.(A)5 |
| 10 | 8115780 | 8115785 | p1.(A)6 |
| 10 | 21827751 | 21827755 | p1.(T)5 |
| 10 | 21884240 | 21884255 | p4.(ATTT)4 |
| 10 | 21884253 | 21884257 | p1.(T)5 |
| 10 | 21962283 | 21962287 | p1.(T)5 |
| 10 | 21962452 | 21962458 | p1.(G)7 |
| 10 | 21962680 | 21962684 | p1.(A)5 |
| 10 | 22002690 | 22002696 | p1.(T)7 |
| 10 | 22015159 | 22015164 | p1.(T)6 |
| 10 | 22019953 | 22019957 | p1.(T)5 |
| 10 | 22021893 | 22021897 | p1.(A)5 |
| 10 | 22021908 | 22021912 | p1.(A)5 |
| 10 | 22022825 | 22022829 | p1.(G)5 |
| 10 | 22024060 | 22024064 | p1.(T)5 |
| 10 | 22029094 | 22029098 | p1.(C)5 |
| 10 | 27037488 | 27037498 | p1.(A)11 |
| 10 | 27037681 | 27037686 | p1.(A)6 |
| 10 | 27040546 | 27040550 | p1.(G)5 |
| 10 | 27040624 | 27040638 | p3.(TGG)5 |
| 10 | 27040637 | 27040641 | p1.(G)5 |
| 10 | 27040715 | 27040719 | p1.(A)5 |
| 10 | 27044646 | 27044650 | p1.(G)5 |
| 10 | 27044652 | 27044656 | p1.(G)5 |
| 10 | 27057880 | 27057884 | p1.(G)5 |
| 10 | 32304596 | 32304600 | p1.(A)5 |
| 10 | 32307245 | 32307249 | p1.(T)5 |
| 10 | 32307420 | 32307424 | p1.(T)5 |
| 10 | 32310012 | 32310016 | p1.(T)5 |
| 10 | 32311193 | 32311198 | p1.(A)6 |
| 10 | 32311826 | 32311832 | p1.(T)7 |
| 10 | 32311883 | 32311887 | p1.(T)5 |
| 10 | 32311973 | 32311977 | p1.(A)5 |
| 10 | 32317434 | 32317439 | p1.(T)6 |
| 10 | 32323677 | 32323683 | p1.(T)7 |
| 10 | 32323685 | 32323689 | p1.(T)5 |
| 10 | 32323691 | 32323695 | p1.(T)5 |
| 10 | 32323704 | 32323708 | p1.(T)5 |
| 10 | 32324604 | 32324608 | p1.(A)5 |
| 10 | 32324876 | 32324880 | p1.(T)5 |
| 10 | 32344763 | 32344767 | p1.(G)5 |
| 10 | 43597993 | 43597997 | p1.(C)5 |
| 10 | 43606646 | 43606651 | p1.(C)6 |
| 10 | 43607569 | 43607573 | p1.(C)5 |
| 10 | 43609003 | 43609007 | p1.(G)5 |
| 10 | 43609018 | 43609022 | p1.(G)5 |
| 10 | 43609043 | 43609047 | p1.(G)5 |
| 10 | 43609915 | 43609919 | p1.(C)5 |
| 10 | 43612076 | 43612080 | p1.(A)5 |
| 10 | 43614972 | 43614976 | p1.(C)5 |
| 10 | 43615516 | 43615520 | p1.(T)5 |
| 10 | 43617431 | 43617435 | p1.(T)5 |
| 10 | 43619159 | 43619163 | p1.(G)5 |
| 10 | 43622120 | 43622125 | p1.(C)6 |
| 10 | 43623553 | 43623557 | p1.(T)5 |
| 10 | 51580868 | 51580872 | p1.(T)5 |
| 10 | 51581255 | 51581263 | p1.(T)9 |
| 10 | 51582894 | 51582898 | p1.(C)5 |
| 10 | 51584605 | 51584609 | p1.(T)5 |
| 10 | 51584652 | 51584656 | p1.(G)5 |
| 10 | 51584695 | 51584699 | p1.(A)5 |
| 10 | 51585043 | 51585047 | p1.(C)5 |
| 10 | 51585146 | 51585155 | p2.(GT)5 |
| 10 | 51585216 | 51585220 | p1.(A)5 |
| 10 | 51585402 | 51585406 | p1.(C)5 |
| 10 | 51586312 | 51586317 | p1.(C)6 |
| 10 | 61592325 | 61592330 | p1.(T)6 |
| 10 | 61665867 | 61665872 | p1.(G)6 |
| 10 | 61666040 | 61666045 | p1.(C)6 |
| 10 | 61666067 | 61666071 | p1.(C)5 |
| 10 | 61666139 | 61666144 | p1.(C)6 |
| 10 | 70332076 | 70332081 | p1.(T)6 |
| 10 | 70332153 | 70332160 | p1.(A)8 |
| 10 | 70332162 | 70332166 | p1.(A)5 |
| 10 | 70332201 | 70332205 | p1.(A)5 |
| 10 | 70332273 | 70332279 | p1.(A)7 |
| 10 | 70332364 | 70332368 | p1.(T)5 |
| 10 | 70332447 | 70332451 | p1.(C)5 |
| 10 | 70332471 | 70332475 | p1.(A)5 |
| 10 | 70332815 | 70332819 | p1.(A)5 |
| 10 | 70332865 | 70332869 | p1.(C)5 |
| 10 | 70333548 | 70333552 | p1.(A)5 |
| 10 | 70333977 | 70333981 | p1.(A)5 |
| 10 | 70404534 | 70404538 | p1.(A)5 |
| 10 | 70404575 | 70404579 | p1.(A)5 |
| 10 | 70404687 | 70404691 | p1.(A)5 |
| 10 | 70404695 | 70404699 | p1.(A)5 |
| 10 | 70404809 | 70404814 | p1.(A)6 |
| 10 | 70404887 | 70404891 | p1.(A)5 |
| 10 | 70405054 | 70405058 | p1.(A)5 |
| 10 | 70405365 | 70405369 | p1.(A)5 |
| 10 | 70405500 | 70405504 | p1.(T)5 |
| 10 | 70405652 | 70405657 | p1.(A)6 |
| 10 | 70405778 | 70405783 | p1.(A)6 |
| 10 | 70405827 | 70405831 | p1.(A)5 |
| 10 | 70405928 | 70405932 | p1.(A)5 |
| 10 | 70405964 | 70405968 | p1.(A)5 |
| 10 | 70406663 | 70406667 | p1.(A)5 |
| 10 | 70406707 | 70406712 | p1.(A)6 |
| 10 | 70411615 | 70411619 | p1.(A)5 |
| 10 | 70412266 | 70412270 | p1.(A)5 |
| 10 | 70426793 | 70426798 | p1.(T)6 |
| 10 | 70426827 | 70426831 | p1.(A)5 |
| 10 | 70446284 | 70446290 | p1.(A)7 |
| 10 | 70446420 | 70446431 | p3.(CAA)4 |
| 10 | 70450596 | 70450600 | p1.(A)5 |
| 10 | 70450626 | 70450630 | p1.(A)5 |
| 10 | 70451094 | 70451098 | p1.(C)5 |
| 10 | 70451161 | 70451165 | p1.(T)5 |
| 10 | 70451314 | 70451318 | p1.(A)5 |
| 10 | 70451408 | 70451412 | p1.(A)5 |
| 10 | 70451582 | 70451586 | p1.(C)5 |
| 10 | 72357844 | 72357848 | p1.(C)5 |
| 10 | 72357859 | 72357863 | p1.(G)5 |
| 10 | 72358049 | 72358054 | p1.(C)6 |
| 10 | 72358189 | 72358194 | p1.(C)6 |
| 10 | 72358309 | 72358313 | p1.(G)5 |
| 10 | 72358622 | 72358633 | p3.(CTT)4 |
| 10 | 72358914 | 72358918 | p1.(G)5 |
| 10 | 76602675 | 76602680 | p1.(A)6 |
| 10 | 76603077 | 76603081 | p1.(G)5 |
| 10 | 76729788 | 76729793 | p1.(T)6 |
| 10 | 76735201 | 76735205 | p1.(G)5 |
| 10 | 76735416 | 76735421 | p1.(T)6 |
| 10 | 76735491 | 76735495 | p1.(A)5 |
| 10 | 76735581 | 76735585 | p1.(C)5 |
| 10 | 76735588 | 76735592 | p1.(C)5 |
| 10 | 76735770 | 76735774 | p1.(A)5 |
| 10 | 76735895 | 76735906 | p3.(CTC)4 |
| 10 | 76735912 | 76735916 | p1.(G)5 |
| 10 | 76735933 | 76735937 | p1.(T)5 |
| 10 | 76735974 | 76735978 | p1.(A)5 |
| 10 | 76736029 | 76736033 | p1.(C)5 |
| 10 | 76737084 | 76737088 | p1.(A)5 |
| 10 | 76737164 | 76737168 | p1.(T)5 |
| 10 | 76741594 | 76741598 | p1.(A)5 |
| 10 | 76744828 | 76744832 | p1.(T)5 |
| 10 | 76744938 | 76744943 | p1.(T)6 |
| 10 | 76744954 | 76744959 | p1.(A)6 |
| 10 | 76781741 | 76781745 | p1.(A)5 |
| 10 | 76781834 | 76781845 | p3.(GAG)4 |
| 10 | 76781906 | 76781929 | p3.(GAA)8 |
| 10 | 76781945 | 76781949 | p1.(C)5 |
| 10 | 76788476 | 76788480 | p1.(A)5 |
| 10 | 76788604 | 76788608 | p1.(A)5 |
| 10 | 76788645 | 76788659 | p3.(GAG)5 |
| 10 | 76788690 | 76788701 | p3.(GAA)4 |
| 10 | 76788718 | 76788722 | p1.(A)5 |
| 10 | 76788748 | 76788752 | p1.(A)5 |
| 10 | 76788775 | 76788779 | p1.(A)5 |
| 10 | 76788956 | 76788961 | p1.(T)6 |
| 10 | 76789101 | 76789105 | p1.(C)5 |
| 10 | 76790190 | 76790194 | p1.(G)5 |
| 10 | 76790223 | 76790228 | p1.(C)6 |
| 10 | 76790731 | 76790735 | p1.(C)5 |
| 10 | 88649875 | 88649879 | p1.(A)5 |
| 10 | 88649922 | 88649927 | p1.(T)6 |
| 10 | 88659632 | 88659636 | p1.(C)5 |
| 10 | 88659789 | 88659794 | p1.(T)6 |
| 10 | 88671986 | 88671991 | p1.(T)6 |
| 10 | 88676877 | 88676882 | p1.(T)6 |
| 10 | 88681365 | 88681369 | p1.(A)5 |
| 10 | 89624200 | 89624204 | p1.(T)5 |
| 10 | 89692758 | 89692762 | p1.(T)5 |
| 10 | 89692949 | 89692953 | p1.(T)5 |
| 10 | 89693003 | 89693007 | p1.(A)5 |
| 10 | 89693017 | 89693022 | p1.(T)6 |
| 10 | 89717770 | 89717775 | p1.(A)6 |
| 10 | 90767548 | 90767552 | p1.(T)5 |
| 10 | 90768708 | 90768714 | p1.(T)7 |
| 10 | 90770283 | 90770291 | p1.(T)9 |
| 10 | 90770365 | 90770371 | p1.(T)7 |
| 10 | 90773868 | 90773872 | p1.(T)5 |
| 10 | 90774094 | 90774099 | p1.(A)6 |
| 10 | 90774212 | 90774216 | p1.(A)5 |
| 10 | 102891275 | 102891280 | p1.(C)6 |
| 10 | 102891476 | 102891480 | p1.(G)5 |
| 10 | 102891496 | 102891500 | p1.(G)5 |
| 10 | 102891511 | 102891515 | p1.(G)5 |
| 10 | 102891557 | 102891568 | p3.(GGC)4 |
| 10 | 102891638 | 102891652 | p3.(GGC)5 |
| 10 | 102891685 | 102891689 | p1.(G)5 |
| 10 | 102891736 | 102891740 | p1.(C)5 |
| 10 | 102893955 | 102893960 | p1.(C)6 |
| 10 | 102893961 | 102893972 | p3.(AAG)4 |
| 10 | 104157118 | 104157122 | p1.(A)5 |
| 10 | 104157745 | 104157749 | p1.(G)5 |
| 10 | 104158134 | 104158139 | p1.(C)6 |
| 10 | 104158163 | 104158168 | p1.(C)6 |
| 10 | 104158489 | 104158493 | p1.(C)5 |
| 10 | 104158555 | 104158559 | p1.(G)5 |
| 10 | 104158585 | 104158589 | p1.(G)5 |
| 10 | 104158597 | 104158601 | p1.(G)5 |
| 10 | 104158626 | 104158630 | p1.(G)5 |
| 10 | 104159413 | 104159422 | p2.(CG)5 |
| 10 | 104159830 | 104159834 | p1.(C)5 |
| 10 | 104159956 | 104159960 | p1.(G)5 |
| 10 | 104160509 | 104160514 | p1.(G)6 |
| 10 | 104160697 | 104160701 | p1.(C)5 |
| 10 | 104160987 | 104160991 | p1.(C)5 |
| 10 | 104161493 | 104161497 | p1.(C)5 |
| 10 | 104162002 | 104162006 | p1.(C)5 |
| 10 | 104162077 | 104162081 | p1.(C)5 |
| 10 | 104162112 | 104162116 | p1.(C)5 |
| 10 | 104162145 | 104162149 | p1.(C)5 |
| 10 | 104162152 | 104162156 | p1.(C)5 |
| 10 | 104162165 | 104162169 | p1.(C)5 |
| 10 | 104263800 | 104263804 | p1.(C)5 |
| 10 | 104263883 | 104263887 | p1.(C)5 |
| 10 | 104263935 | 104263939 | p1.(C)5 |
| 10 | 104263952 | 104263956 | p1.(C)5 |
| 10 | 104263974 | 104263980 | p1.(C)7 |
| 10 | 104264000 | 104264004 | p1.(C)5 |
| 10 | 104268916 | 104268921 | p1.(T)6 |
| 10 | 104356981 | 104356986 | p1.(C)6 |
| 10 | 104849327 | 104849331 | p1.(C)5 |
| 10 | 104849337 | 104849341 | p1.(C)5 |
| 10 | 104849436 | 104849450 | p3.(TCC)5 |
| 10 | 104849491 | 104849496 | p1.(G)6 |
| 10 | 104849674 | 104849679 | p1.(A)6 |
| 10 | 104850467 | 104850471 | p1.(A)5 |
| 10 | 104852946 | 104852951 | p1.(A)6 |
| 10 | 104852993 | 104852997 | p1.(A)5 |
| 10 | 104854138 | 104854143 | p1.(A)6 |
| 10 | 104865462 | 104865466 | p1.(C)5 |
| 10 | 104866337 | 104866341 | p1.(A)5 |
| 10 | 104934639 | 104934643 | p1.(T)5 |
| 10 | 114286893 | 114286897 | p1.(G)5 |
| 10 | 114297997 | 114298001 | p1.(T)5 |
| 10 | 114428722 | 114428726 | p1.(A)5 |
| 10 | 114575121 | 114575125 | p1.(T)5 |
| 10 | 114710508 | 114710516 | p1.(A)9 |
| 10 | 114900984 | 114900988 | p1.(C)5 |
| 10 | 114903755 | 114903759 | p1.(C)5 |
| 10 | 114911500 | 114911504 | p1.(A)5 |
| 10 | 114925317 | 114925325 | p1.(A)9 |
| 10 | 114925402 | 114925407 | p1.(C)6 |
| 10 | 114925494 | 114925498 | p1.(C)5 |
| 10 | 114925597 | 114925601 | p1.(C)5 |
| 10 | 114925622 | 114925626 | p1.(C)5 |
| 10 | 123239442 | 123239447 | p1.(A)6 |
| 10 | 123243323 | 123243327 | p1.(A)5 |
| 10 | 123244965 | 123244969 | p1.(A)5 |
| 10 | 123245002 | 123245006 | p1.(C)5 |
| 10 | 123247515 | 123247519 | p1.(T)5 |
| 10 | 123256046 | 123256050 | p1.(T)5 |
| 10 | 123263369 | 123263373 | p1.(G)5 |
| 10 | 123274648 | 123274652 | p1.(G)5 |
| 10 | 123310847 | 123310851 | p1.(T)5 |
| 10 | 123310879 | 123310884 | p1.(C)6 |
| 11 | 532622 | 532626 | p1.(C)5 |
| 11 | 534295 | 534306 | p3.(CCA)4 |
| 11 | 3022322 | 3022326 | p1.(A)5 |
| 11 | 3022339 | 3022343 | p1.(C)5 |
| 11 | 3023251 | 3023256 | p1.(G)6 |
| 11 | 3033426 | 3033430 | p1.(T)5 |
| 11 | 3039177 | 3039181 | p1.(A)5 |
| 11 | 3039746 | 3039751 | p1.(G)6 |
| 11 | 3039858 | 3039862 | p1.(T)5 |
| 11 | 3039888 | 3039893 | p1.(T)6 |
| 11 | 3040398 | 3040402 | p1.(G)5 |
| 11 | 3050562 | 3050566 | p1.(C)5 |
| 11 | 3059386 | 3059390 | p1.(T)5 |
| 11 | 3062186 | 3062190 | p1.(T)5 |
| 11 | 3697615 | 3697619 | p1.(A)5 |
| 11 | 3720389 | 3720395 | p1.(T)7 |
| 11 | 3722033 | 3722037 | p1.(T)5 |
| 11 | 3726589 | 3726593 | p1.(A)5 |
| 11 | 3740668 | 3740672 | p1.(T)5 |
| 11 | 3744609 | 3744613 | p1.(A)5 |
| 11 | 3752812 | 3752816 | p1.(A)5 |
| 11 | 3765751 | 3765756 | p1.(G)6 |
| 11 | 3784225 | 3784229 | p1.(A)5 |
| 11 | 3789983 | 3789987 | p1.(A)5 |
| 11 | 3800491 | 3800495 | p1.(A)5 |
| 11 | 8246089 | 8246100 | p4.(TGGC)3 |
| 11 | 14480238 | 14480243 | p1.(A)6 |
| 11 | 14496048 | 14496052 | p1.(T)5 |
| 11 | 14501268 | 14501281 | p1.(A)14 |
| 11 | 14502418 | 14502422 | p1.(T)5 |
| 11 | 14502646 | 14502651 | p1.(A)6 |
| 11 | 32410683 | 32410687 | p1.(T)5 |
| 11 | 32439208 | 32439212 | p1.(A)5 |
| 11 | 32449555 | 32449559 | p1.(G)5 |
| 11 | 32449577 | 32449581 | p1.(G)5 |
| 11 | 32456485 | 32456496 | p3.(GGC)4 |
| 11 | 32456497 | 32456501 | p1.(G)5 |
| 11 | 32456560 | 32456564 | p1.(G)5 |
| 11 | 32456619 | 32456630 | p3.(GCC)4 |
| 11 | 33881115 | 33881119 | p1.(A)5 |
| 11 | 33886331 | 33886336 | p1.(G)6 |
| 11 | 33891192 | 33891196 | p1.(G)5 |
| 11 | 33891230 | 33891244 | p3.(GCT)5 |
| 11 | 44129501 | 44129506 | p1.(G)6 |
| 11 | 44129596 | 44129600 | p1.(A)5 |
| 11 | 44130793 | 44130797 | p1.(C)5 |
| 11 | 44151655 | 44151659 | p1.(C)5 |
| 11 | 44219553 | 44219557 | p1.(A)5 |
| 11 | 44228334 | 44228338 | p1.(T)5 |
| 11 | 44254034 | 44254038 | p1.(T)5 |
| 11 | 44254051 | 44254055 | p1.(G)5 |
| 11 | 46299640 | 46299644 | p1.(G)5 |
| 11 | 46299728 | 46299732 | p1.(G)5 |
| 11 | 46321677 | 46321681 | p1.(C)5 |
| 11 | 46329463 | 46329467 | p1.(C)5 |
| 11 | 46329542 | 46329546 | p1.(C)5 |
| 11 | 46332609 | 46332614 | p1.(C)6 |
| 11 | 46332663 | 46332668 | p1.(C)6 |
| 11 | 46333965 | 46333969 | p1.(C)5 |
| 11 | 46339001 | 46339005 | p1.(C)5 |
| 11 | 46341895 | 46341899 | p1.(C)5 |
| 11 | 46341922 | 46341927 | p1.(G)6 |
| 11 | 47238032 | 47238037 | p1.(T)6 |
| 11 | 47238424 | 47238428 | p1.(T)5 |
| 11 | 47238467 | 47238471 | p1.(C)5 |
| 11 | 47238541 | 47238545 | p1.(G)5 |
| 11 | 47254486 | 47254490 | p1.(T)5 |
| 11 | 47256329 | 47256333 | p1.(A)5 |
| 11 | 47256906 | 47256910 | p1.(C)5 |
| 11 | 57427437 | 57427441 | p1.(G)5 |
| 11 | 57428440 | 57428444 | p1.(C)5 |
| 11 | 57428477 | 57428481 | p1.(G)5 |
| 11 | 57428542 | 57428546 | p1.(T)5 |
| 11 | 61205469 | 61205473 | p1.(T)5 |
| 11 | 61205478 | 61205482 | p1.(T)5 |
| 11 | 61213475 | 61213479 | p1.(A)5 |
| 11 | 61213481 | 61213485 | p1.(A)5 |
| 11 | 61213566 | 61213570 | p1.(G)5 |
| 11 | 64003758 | 64003762 | p1.(G)5 |
| 11 | 64004663 | 64004670 | p1.(A)8 |
| 11 | 64004948 | 64004952 | p1.(C)5 |
| 11 | 64005041 | 64005045 | p1.(C)5 |
| 11 | 64005904 | 64005908 | p1.(G)5 |
| 11 | 64572093 | 64572099 | p1.(G)7 |
| 11 | 64572161 | 64572165 | p1.(G)5 |
| 11 | 64572679 | 64572683 | p1.(G)5 |
| 11 | 64573118 | 64573122 | p1.(C)5 |
| 11 | 64573691 | 64573695 | p1.(G)5 |
| 11 | 64574476 | 64574480 | p1.(C)5 |
| 11 | 64575157 | 64575163 | p1.(G)7 |
| 11 | 64575580 | 64575584 | p1.(G)5 |
| 11 | 64577250 | 64577254 | p1.(C)5 |
| 11 | 64577375 | 64577379 | p1.(G)5 |
| 11 | 69465873 | 69465882 | p2.(CT)5 |
| 11 | 69465988 | 69466002 | p3.(GAG)5 |
| 11 | 69518488 | 69518492 | p1.(G)5 |
| 11 | 69518572 | 69518576 | p1.(G)5 |
| 11 | 69588898 | 69588902 | p1.(G)5 |
| 11 | 69589770 | 69589774 | p1.(C)5 |
| 11 | 69625177 | 69625181 | p1.(C)5 |
| 11 | 69625188 | 69625192 | p1.(G)5 |
| 11 | 71715035 | 71715039 | p1.(G)5 |
| 11 | 71715808 | 71715812 | p1.(G)5 |
| 11 | 71716313 | 71716318 | p1.(G)6 |
| 11 | 71718342 | 71718347 | p1.(G)6 |
| 11 | 71718403 | 71718408 | p1.(G)6 |
| 11 | 71723497 | 71723501 | p1.(G)5 |
| 11 | 71724190 | 71724194 | p1.(C)5 |
| 11 | 71724368 | 71724372 | p1.(C)5 |
| 11 | 71724810 | 71724814 | p1.(C)5 |
| 11 | 71724878 | 71724882 | p1.(T)5 |
| 11 | 71726491 | 71726495 | p1.(T)5 |
| 11 | 71730666 | 71730670 | p1.(G)5 |
| 11 | 71735404 | 71735408 | p1.(A)5 |
| 11 | 76164345 | 76164349 | p1.(T)5 |
| 11 | 76169215 | 76169220 | p1.(T)6 |
| 11 | 76169411 | 76169415 | p1.(A)5 |
| 11 | 76174889 | 76174893 | p1.(A)5 |
| 11 | 76175070 | 76175074 | p1.(A)5 |
| 11 | 76207304 | 76207308 | p1.(C)5 |
| 11 | 76227322 | 76227327 | p1.(G)6 |
| 11 | 76234303 | 76234307 | p1.(A)5 |
| 11 | 76237646 | 76237650 | p1.(A)5 |
| 11 | 76248827 | 76248832 | p1.(T)6 |
| 11 | 76248845 | 76248849 | p1.(A)5 |
| 11 | 76255291 | 76255296 | p1.(T)6 |
| 11 | 76255462 | 76255466 | p1.(C)5 |
| 11 | 76255598 | 76255602 | p1.(A)5 |
| 11 | 76255708 | 76255712 | p1.(C)5 |
| 11 | 76255788 | 76255792 | p1.(A)5 |
| 11 | 76255811 | 76255816 | p1.(C)6 |
| 11 | 76257075 | 76257079 | p1.(C)5 |
| 11 | 85685858 | 85685863 | p1.(A)6 |
| 11 | 85692159 | 85692163 | p1.(T)5 |
| 11 | 85692227 | 85692231 | p1.(C)5 |
| 11 | 85722158 | 85722162 | p1.(T)5 |
| 11 | 85722175 | 85722179 | p1.(T)5 |
| 11 | 85723442 | 85723446 | p1.(A)5 |
| 11 | 85742661 | 85742671 | p1.(A)6(T)5 |
| 11 | 85779704 | 85779708 | p1.(T)5 |
| 11 | 85779875 | 85779879 | p1.(C)5 |
| 11 | 94189473 | 94189479 | p1.(T)7 |
| 11 | 94192633 | 94192638 | p1.(T)6 |
| 11 | 94197282 | 94197287 | p1.(T)6 |
| 11 | 94197308 | 94197313 | p1.(A)6 |
| 11 | 94203625 | 94203629 | p1.(A)5 |
| 11 | 94203691 | 94203695 | p1.(A)5 |
| 11 | 94203731 | 94203735 | p1.(A)5 |
| 11 | 94204818 | 94204822 | p1.(T)5 |
| 11 | 94204884 | 94204888 | p1.(A)5 |
| 11 | 94209523 | 94209528 | p1.(T)6 |
| 11 | 94211932 | 94211936 | p1.(T)5 |
| 11 | 94219083 | 94219094 | p4.(TACT)3 |
| 11 | 94219218 | 94219222 | p1.(A)5 |
| 11 | 95713133 | 95713137 | p1.(A)5 |
| 11 | 95825204 | 95825221 | p3.(TGC)6 |
| 11 | 95825240 | 95825254 | p3.(TGC)5 |
| 11 | 95825261 | 95825275 | p3.(TGC)5 |
| 11 | 95825357 | 95825371 | p3.(TGC)5 |
| 11 | 95825375 | 95825413 | p3.(TGC)13 |
| 11 | 95825707 | 95825711 | p1.(G)5 |
| 11 | 95826041 | 95826045 | p1.(G)5 |
| 11 | 95826328 | 95826332 | p1.(A)5 |
| 11 | 96074680 | 96074684 | p1.(G)5 |
| 11 | 96074734 | 96074738 | p1.(G)5 |
| 11 | 96074804 | 96074808 | p1.(C)5 |
| 11 | 96074983 | 96074987 | p1.(C)5 |
| 11 | 96075011 | 96075017 | p1.(C)7 |
| 11 | 96075030 | 96075034 | p1.(G)5 |
| 11 | 96075040 | 96075044 | p1.(G)5 |
| 11 | 96075053 | 96075057 | p1.(C)5 |
| 11 | 102195499 | 102195503 | p1.(A)5 |
| 11 | 102195699 | 102195703 | p1.(T)5 |
| 11 | 102196080 | 102196084 | p1.(T)5 |
| 11 | 102206685 | 102206690 | p1.(T)6 |
| 11 | 102206733 | 102206737 | p1.(T)5 |
| 11 | 108098310 | 108098317 | p1.(T)8 |
| 11 | 108098490 | 108098494 | p1.(T)5 |
| 11 | 108098609 | 108098613 | p1.(T)5 |
| 11 | 108099906 | 108099910 | p1.(T)5 |
| 11 | 108099993 | 108099997 | p1.(A)5 |
| 11 | 108114662 | 108114676 | p1.(T)15 |
| 11 | 108114808 | 108114812 | p1.(T)5 |
| 11 | 108114817 | 108114823 | p1.(T)7 |
| 11 | 108115748 | 108115752 | p1.(A)5 |
| 11 | 108119661 | 108119665 | p1.(T)5 |
| 11 | 108121411 | 108121425 | p1.(T)15 |
| 11 | 108122621 | 108122625 | p1.(A)5 |
| 11 | 108123531 | 108123535 | p1.(T)5 |
| 11 | 108123593 | 108123597 | p1.(A)5 |
| 11 | 108123616 | 108123621 | p1.(T)6 |
| 11 | 108124552 | 108124556 | p1.(A)5 |
| 11 | 108124626 | 108124630 | p1.(T)5 |
| 11 | 108124773 | 108124785 | p1.(T)6(A)7 |
| 11 | 108128247 | 108128251 | p1.(A)5 |
| 11 | 108129740 | 108129744 | p1.(T)5 |
| 11 | 108137910 | 108137914 | p1.(A)5 |
| 11 | 108139124 | 108139128 | p1.(T)5 |
| 11 | 108139130 | 108139134 | p1.(T)5 |
| 11 | 108141956 | 108141970 | p1.(T)15 |
| 11 | 108142051 | 108142055 | p1.(A)5 |
| 11 | 108143340 | 108143344 | p1.(T)5 |
| 11 | 108151714 | 108151718 | p1.(T)5 |
| 11 | 108151825 | 108151829 | p1.(A)5 |
| 11 | 108151890 | 108151894 | p1.(A)5 |
| 11 | 108154947 | 108154951 | p1.(T)5 |
| 11 | 108159797 | 108159801 | p1.(A)5 |
| 11 | 108160451 | 108160455 | p1.(A)5 |
| 11 | 108164137 | 108164141 | p1.(T)5 |
| 11 | 108164164 | 108164169 | p1.(A)6 |
| 11 | 108164210 | 108164214 | p1.(A)5 |
| 11 | 108168095 | 108168099 | p1.(A)5 |
| 11 | 108172365 | 108172371 | p1.(T)7 |
| 11 | 108172409 | 108172413 | p1.(A)5 |
| 11 | 108172510 | 108172515 | p1.(A)6 |
| 11 | 108173580 | 108173584 | p1.(T)5 |
| 11 | 108173696 | 108173701 | p1.(T)6 |
| 11 | 108175510 | 108175514 | p1.(T)5 |
| 11 | 108178635 | 108178639 | p1.(T)5 |
| 11 | 108178656 | 108178661 | p1.(A)6 |
| 11 | 108180876 | 108180880 | p1.(T)5 |
| 11 | 108180904 | 108180908 | p1.(T)5 |
| 11 | 108186846 | 108186851 | p1.(T)6 |
| 11 | 108192014 | 108192019 | p1.(T)6 |
| 11 | 108196880 | 108196885 | p1.(A)6 |
| 11 | 108196958 | 108196965 | p1.(T)8 |
| 11 | 108198492 | 108198496 | p1.(T)5 |
| 11 | 108199804 | 108199808 | p1.(A)5 |
| 11 | 108202155 | 108202162 | p1.(T)8 |
| 11 | 108202201 | 108202205 | p1.(T)5 |
| 11 | 108202632 | 108202636 | p1.(C)5 |
| 11 | 108202729 | 108202733 | p1.(A)5 |
| 11 | 108203475 | 108203480 | p1.(T)6 |
| 11 | 108203633 | 108203640 | p1.(T)8 |
| 11 | 108205782 | 108205786 | p1.(A)5 |
| 11 | 108216477 | 108216483 | p1.(A)7 |
| 11 | 108216561 | 108216565 | p1.(A)5 |
| 11 | 108217994 | 108217999 | p1.(T)6 |
| 11 | 108236039 | 108236043 | p1.(T)5 |
| 11 | 108236191 | 108236195 | p1.(A)5 |
| 11 | 108236294 | 108236298 | p1.(T)5 |
| 11 | 108535865 | 108535879 | p3.(CGC)5 |
| 11 | 108535953 | 108535958 | p1.(A)6 |
| 11 | 108535977 | 108535981 | p1.(A)5 |
| 11 | 108544183 | 108544189 | p1.(T)7 |
| 11 | 108544239 | 108544245 | p1.(A)7 |
| 11 | 108547804 | 108547808 | p1.(T)5 |
| 11 | 108547859 | 108547863 | p1.(G)5 |
| 11 | 108550090 | 108550094 | p1.(T)5 |
| 11 | 108550244 | 108550248 | p1.(A)5 |
| 11 | 108550272 | 108550276 | p1.(A)5 |
| 11 | 108559710 | 108559715 | p1.(A)6 |
| 11 | 108559733 | 108559737 | p1.(T)5 |
| 11 | 108559769 | 108559775 | p1.(T)7 |
| 11 | 108577455 | 108577459 | p1.(T)5 |
| 11 | 108577510 | 108577514 | p1.(A)5 |
| 11 | 108586617 | 108586621 | p1.(A)5 |
| 11 | 108586637 | 108586642 | p1.(A)6 |
| 11 | 108586702 | 108586706 | p1.(T)5 |
| 11 | 108593903 | 108593907 | p1.(A)5 |
| 11 | 108593913 | 108593917 | p1.(A)5 |
| 11 | 108593971 | 108593982 | p3.(GAA)4 |
| 11 | 111225266 | 111225270 | p1.(G)5 |
| 11 | 111249888 | 111249892 | p1.(T)5 |
| 11 | 111959582 | 111959586 | p1.(T)5 |
| 11 | 111965518 | 111965522 | p1.(T)5 |
| 11 | 113934034 | 113934038 | p1.(A)5 |
| 11 | 113934713 | 113934717 | p1.(G)5 |
| 11 | 113934826 | 113934830 | p1.(G)5 |
| 11 | 113934944 | 113934948 | p1.(C)5 |
| 11 | 113934966 | 113934970 | p1.(C)5 |
| 11 | 113934995 | 113934999 | p1.(C)5 |
| 11 | 113935136 | 113935141 | p1.(G)6 |
| 11 | 113935186 | 113935190 | p1.(G)5 |
| 11 | 117023146 | 117023151 | p1.(T)6 |
| 11 | 117031852 | 117031858 | p1.(T)7 |
| 11 | 117031874 | 117031879 | p1.(T)6 |
| 11 | 117031909 | 117031913 | p1.(G)5 |
| 11 | 117038254 | 117038258 | p1.(G)5 |
| 11 | 117038323 | 117038327 | p1.(G)5 |
| 11 | 117079626 | 117079631 | p1.(G)6 |
| 11 | 117089206 | 117089211 | p1.(G)6 |
| 11 | 117100408 | 117100413 | p1.(C)6 |
| 11 | 117100518 | 117100523 | p1.(G)6 |
| 11 | 118307276 | 118307280 | p1.(G)5 |
| 11 | 118307279 | 118307290 | p3.(GGC)4 |
| 11 | 118307291 | 118307296 | p1.(G)6 |
| 11 | 118307453 | 118307457 | p1.(G)5 |
| 11 | 118307639 | 118307643 | p1.(G)5 |
| 11 | 118307664 | 118307668 | p1.(G)5 |
| 11 | 118342567 | 118342571 | p1.(A)5 |
| 11 | 118342634 | 118342640 | p1.(A)7 |
| 11 | 118342685 | 118342690 | p1.(A)6 |
| 11 | 118342759 | 118342763 | p1.(G)5 |
| 11 | 118342966 | 118342970 | p1.(A)5 |
| 11 | 118343011 | 118343021 | p1.(A)6(G)5 |
| 11 | 118343037 | 118343041 | p1.(A)5 |
| 11 | 118343084 | 118343088 | p1.(A)5 |
| 11 | 118343253 | 118343257 | p1.(A)5 |
| 11 | 118343504 | 118343509 | p1.(A)6 |
| 11 | 118343529 | 118343534 | p1.(C)6 |
| 11 | 118343660 | 118343664 | p1.(T)5 |
| 11 | 118343831 | 118343835 | p1.(C)5 |
| 11 | 118343872 | 118343876 | p1.(T)5 |
| 11 | 118344076 | 118344080 | p1.(A)5 |
| 11 | 118344186 | 118344192 | p1.(C)7 |
| 11 | 118344308 | 118344312 | p1.(A)5 |
| 11 | 118344435 | 118344439 | p1.(C)5 |
| 11 | 118344494 | 118344503 | p2.(AG)5 |
| 11 | 118344554 | 118344558 | p1.(A)5 |
| 11 | 118344656 | 118344661 | p1.(A)6 |
| 11 | 118344783 | 118344788 | p1.(A)6 |
| 11 | 118344955 | 118344960 | p1.(A)6 |
| 11 | 118352418 | 118352422 | p1.(T)5 |
| 11 | 118352435 | 118352439 | p1.(A)5 |
| 11 | 118352447 | 118352451 | p1.(A)5 |
| 11 | 118352780 | 118352784 | p1.(A)5 |
| 11 | 118354899 | 118354903 | p1.(A)5 |
| 11 | 118354983 | 118354988 | p1.(A)6 |
| 11 | 118360971 | 118360976 | p1.(A)6 |
| 11 | 118361941 | 118361952 | p3.(ATG)4 |
| 11 | 118362558 | 118362562 | p1.(A)5 |
| 11 | 118363830 | 118363834 | p1.(C)5 |
| 11 | 118364990 | 118364994 | p1.(T)5 |
| 11 | 118365075 | 118365080 | p1.(A)6 |
| 11 | 118365422 | 118365426 | p1.(T)5 |
| 11 | 118365442 | 118365446 | p1.(A)5 |
| 11 | 118367017 | 118367021 | p1.(C)5 |
| 11 | 118369078 | 118369082 | p1.(T)5 |
| 11 | 118369199 | 118369204 | p1.(A)6 |
| 11 | 118373442 | 118373446 | p1.(A)5 |
| 11 | 118373904 | 118373909 | p1.(A)6 |
| 11 | 118373951 | 118373956 | p1.(T)6 |
| 11 | 118374197 | 118374201 | p1.(A)5 |
| 11 | 118374222 | 118374226 | p1.(A)5 |
| 11 | 118374498 | 118374503 | p1.(T)6 |
| 11 | 118374752 | 118374756 | p1.(A)5 |
| 11 | 118374839 | 118374843 | p1.(A)5 |
| 11 | 118374939 | 118374943 | p1.(A)5 |
| 11 | 118375010 | 118375024 | p5.(AGCTC)3 |
| 11 | 118375057 | 118375061 | p1.(C)5 |
| 11 | 118375068 | 118375072 | p1.(A)5 |
| 11 | 118375279 | 118375283 | p1.(A)5 |
| 11 | 118375294 | 118375298 | p1.(T)5 |
| 11 | 118375507 | 118375511 | p1.(A)5 |
| 11 | 118375915 | 118375920 | p1.(A)6 |
| 11 | 118376310 | 118376315 | p1.(A)6 |
| 11 | 118376474 | 118376478 | p1.(A)5 |
| 11 | 118376857 | 118376861 | p1.(C)5 |
| 11 | 118377153 | 118377157 | p1.(G)5 |
| 11 | 118377230 | 118377234 | p1.(A)5 |
| 11 | 118379840 | 118379844 | p1.(T)5 |
| 11 | 118380758 | 118380763 | p1.(A)6 |
| 11 | 118380780 | 118380784 | p1.(T)5 |
| 11 | 118390461 | 118390466 | p1.(C)6 |
| 11 | 118391565 | 118391570 | p1.(A)6 |
| 11 | 118392900 | 118392904 | p1.(C)5 |
| 11 | 118622560 | 118622570 | p1.(T)6(A)5 |
| 11 | 118622625 | 118622630 | p1.(A)6 |
| 11 | 118622653 | 118622657 | p1.(T)5 |
| 11 | 118622671 | 118622677 | p1.(A)7 |
| 11 | 118625599 | 118625604 | p1.(A)6 |
| 11 | 118626099 | 118626104 | p1.(T)6 |
| 11 | 118626216 | 118626226 | p1.(A)11 |
| 11 | 118629513 | 118629517 | p1.(T)5 |
| 11 | 118629614 | 118629621 | p1.(G)8 |
| 11 | 118630686 | 118630690 | p1.(T)5 |
| 11 | 118639005 | 118639010 | p1.(T)6 |
| 11 | 118650374 | 118650378 | p1.(A)5 |
| 11 | 118651873 | 118651877 | p1.(T)5 |
| 11 | 118656821 | 118656825 | p1.(T)5 |
| 11 | 118656857 | 118656861 | p1.(C)5 |
| 11 | 119077161 | 119077165 | p1.(G)5 |
| 11 | 119077179 | 119077183 | p1.(G)5 |
| 11 | 119077233 | 119077253 | p3.(CAC)7 |
| 11 | 119144566 | 119144570 | p1.(C)5 |
| 11 | 119144700 | 119144704 | p1.(T)5 |
| 11 | 119145604 | 119145609 | p1.(T)6 |
| 11 | 119148860 | 119148867 | p1.(T)8 |
| 11 | 119149356 | 119149373 | p3.(ATG)6 |
| 11 | 119155727 | 119155731 | p1.(C)5 |
| 11 | 119156070 | 119156074 | p1.(C)5 |
| 11 | 119169057 | 119169061 | p1.(T)5 |
| 11 | 119170435 | 119170439 | p1.(A)5 |
| 11 | 120276837 | 120276842 | p1.(A)6 |
| 11 | 120298953 | 120298957 | p1.(G)5 |
| 11 | 120310939 | 120310944 | p1.(T)6 |
| 11 | 120319849 | 120319853 | p1.(A)5 |
| 11 | 120319887 | 120319891 | p1.(C)5 |
| 11 | 120322399 | 120322403 | p1.(T)5 |
| 11 | 120328775 | 120328780 | p1.(T)6 |
| 11 | 120329994 | 120329999 | p1.(T)6 |
| 11 | 120331368 | 120331372 | p1.(T)5 |
| 11 | 120336039 | 120336043 | p1.(A)5 |
| 11 | 120340100 | 120340105 | p1.(A)6 |
| 11 | 120345283 | 120345287 | p1.(A)5 |
| 11 | 120347360 | 120347365 | p1.(T)6 |
| 11 | 120351016 | 120351020 | p1.(G)5 |
| 11 | 120351043 | 120351048 | p1.(T)6 |
| 11 | 120352143 | 120352147 | p1.(C)5 |
| 11 | 125505317 | 125505321 | p1.(T)5 |
| 11 | 125505378 | 125505386 | p1.(A)9 |
| 11 | 125505404 | 125505410 | p1.(A)7 |
| 11 | 125513679 | 125513684 | p1.(T)6 |
| 11 | 125513691 | 125513695 | p1.(A)5 |
| 11 | 125523746 | 125523750 | p1.(T)5 |
| 11 | 128564127 | 128564132 | p1.(G)6 |
| 11 | 128628138 | 128628142 | p1.(C)5 |
| 11 | 128638115 | 128638120 | p1.(C)6 |
| 11 | 128642763 | 128642767 | p1.(T)5 |
| 11 | 128680557 | 128680561 | p1.(A)5 |
| 11 | 128680798 | 128680802 | p1.(C)5 |
| 11 | 128680805 | 128680810 | p1.(G)6 |
| 11 | 128680832 | 128680836 | p1.(C)5 |
| 11 | 128781906 | 128781910 | p1.(G)5 |
| 11 | 128781918 | 128781922 | p1.(C)5 |
| 11 | 128786512 | 128786517 | p1.(C)6 |
| 11 | 128786524 | 128786529 | p1.(G)6 |
| 12 | 402031 | 402035 | p1.(T)5 |
| 12 | 402100 | 402104 | p1.(T)5 |
| 12 | 402240 | 402244 | p1.(A)5 |
| 12 | 402340 | 402344 | p1.(A)5 |
| 12 | 404967 | 404972 | p1.(A)6 |
| 12 | 416644 | 416648 | p1.(T)5 |
| 12 | 416676 | 416681 | p1.(T)6 |
| 12 | 416953 | 416960 | p1.(T)8 |
| 12 | 417142 | 417146 | p1.(T)5 |
| 12 | 419044 | 419048 | p1.(T)5 |
| 12 | 419050 | 419054 | p1.(T)5 |
| 12 | 419071 | 419076 | p1.(T)6 |
| 12 | 419084 | 419088 | p1.(T)5 |
| 12 | 419118 | 419122 | p1.(G)5 |
| 12 | 419135 | 419139 | p1.(A)5 |
| 12 | 420234 | 420238 | p1.(A)5 |
| 12 | 430200 | 430204 | p1.(A)5 |
| 12 | 431595 | 431599 | p1.(T)5 |
| 12 | 431738 | 431742 | p1.(A)5 |
| 12 | 442737 | 442741 | p1.(G)5 |
| 12 | 442811 | 442816 | p1.(C)6 |
| 12 | 443456 | 443460 | p1.(A)5 |
| 12 | 461499 | 461504 | p1.(A)6 |
| 12 | 463406 | 463415 | p1.(A)10 |
| 12 | 464424 | 464428 | p1.(A)5 |
| 12 | 465650 | 465654 | p1.(A)5 |
| 12 | 465706 | 465711 | p1.(A)6 |
| 12 | 472228 | 472232 | p1.(T)5 |
| 12 | 493223 | 493227 | p1.(T)5 |
| 12 | 498235 | 498240 | p1.(C)6 |
| 12 | 498268 | 498282 | p1.(G)15 |
| 12 | 1137352 | 1137356 | p1.(G)5 |
| 12 | 1192721 | 1192725 | p1.(A)5 |
| 12 | 1219363 | 1219367 | p1.(A)5 |
| 12 | 1219505 | 1219509 | p1.(A)5 |
| 12 | 1221435 | 1221439 | p1.(A)5 |
| 12 | 1250834 | 1250838 | p1.(A)5 |
| 12 | 1291166 | 1291171 | p1.(A)6 |
| 12 | 1291191 | 1291195 | p1.(A)5 |
| 12 | 1292501 | 1292505 | p1.(A)5 |
| 12 | 1292582 | 1292586 | p1.(A)5 |
| 12 | 1292595 | 1292600 | p1.(A)6 |
| 12 | 1299088 | 1299097 | p2.(GA)5 |
| 12 | 1345991 | 1345997 | p1.(A)7 |
| 12 | 1372324 | 1372328 | p1.(A)5 |
| 12 | 1481028 | 1481032 | p1.(A)5 |
| 12 | 1553714 | 1553720 | p1.(T)7 |
| 12 | 1553817 | 1553821 | p1.(C)5 |
| 12 | 1553909 | 1553913 | p1.(G)5 |
| 12 | 4383409 | 4383414 | p1.(G)6 |
| 12 | 4479552 | 4479556 | p1.(C)5 |
| 12 | 4479697 | 4479701 | p1.(G)5 |
| 12 | 4479749 | 4479753 | p1.(G)5 |
| 12 | 4479769 | 4479773 | p1.(G)5 |
| 12 | 4479807 | 4479811 | p1.(G)5 |
| 12 | 4481766 | 4481770 | p1.(A)5 |
| 12 | 4481872 | 4481876 | p1.(A)5 |
| 12 | 4488739 | 4488743 | p1.(C)5 |
| 12 | 6777019 | 6777023 | p1.(C)5 |
| 12 | 6777029 | 6777034 | p1.(G)6 |
| 12 | 6777070 | 6777111 | p3.(TGC)14 |
| 12 | 6777205 | 6777216 | p3.(GCT)4 |
| 12 | 6787521 | 6787525 | p1.(G)5 |
| 12 | 6787601 | 6787605 | p1.(G)5 |
| 12 | 6788698 | 6788703 | p1.(A)6 |
| 12 | 11803022 | 11803027 | p1.(A)6 |
| 12 | 11803097 | 11803101 | p1.(A)5 |
| 12 | 11905374 | 11905378 | p1.(T)5 |
| 12 | 12006348 | 12006352 | p1.(T)5 |
| 12 | 12006418 | 12006423 | p1.(T)6 |
| 12 | 12022376 | 12022380 | p1.(C)5 |
| 12 | 12022502 | 12022507 | p1.(C)6 |
| 12 | 12022665 | 12022669 | p1.(C)5 |
| 12 | 12022734 | 12022738 | p1.(C)5 |
| 12 | 12871044 | 12871048 | p1.(C)5 |
| 12 | 12871053 | 12871058 | p1.(C)6 |
| 12 | 12874045 | 12874049 | p1.(A)5 |
| 12 | 12874095 | 12874099 | p1.(A)5 |
| 12 | 12874183 | 12874187 | p1.(T)5 |
| 12 | 25362678 | 25362682 | p1.(A)5 |
| 12 | 25362710 | 25362715 | p1.(A)6 |
| 12 | 25362760 | 25362764 | p1.(T)5 |
| 12 | 25362769 | 25362773 | p1.(T)5 |
| 12 | 25398338 | 25398342 | p1.(A)5 |
| 12 | 46123563 | 46123568 | p1.(G)6 |
| 12 | 46123592 | 46123602 | p1.(T)6(A)5 |
| 12 | 46123612 | 46123617 | p1.(A)6 |
| 12 | 46123837 | 46123843 | p1.(A)7 |
| 12 | 46211615 | 46211619 | p1.(A)5 |
| 12 | 46231095 | 46231099 | p1.(T)5 |
| 12 | 46231185 | 46231189 | p1.(T)5 |
| 12 | 46231273 | 46231277 | p1.(T)5 |
| 12 | 46231462 | 46231466 | p1.(A)5 |
| 12 | 46243567 | 46243571 | p1.(T)5 |
| 12 | 46244454 | 46244458 | p1.(C)5 |
| 12 | 46244967 | 46244971 | p1.(C)5 |
| 12 | 46245162 | 46245166 | p1.(C)5 |
| 12 | 46245318 | 46245322 | p1.(G)5 |
| 12 | 46245609 | 46245613 | p1.(C)5 |
| 12 | 46246026 | 46246030 | p1.(A)5 |
| 12 | 46254574 | 46254578 | p1.(T)5 |
| 12 | 46254728 | 46254732 | p1.(A)5 |
| 12 | 46285553 | 46285558 | p1.(T)6 |
| 12 | 46285708 | 46285712 | p1.(T)5 |
| 12 | 46285788 | 46285792 | p1.(A)5 |
| 12 | 46287216 | 46287220 | p1.(G)5 |
| 12 | 46298706 | 46298712 | p1.(T)7 |
| 12 | 46298836 | 46298840 | p1.(A)5 |
| 12 | 46298861 | 46298865 | p1.(A)5 |
| 12 | 46298884 | 46298888 | p1.(G)5 |
| 12 | 49415529 | 49415533 | p1.(G)5 |
| 12 | 49415856 | 49415867 | p3.(GAT)4 |
| 12 | 49416586 | 49416590 | p1.(G)5 |
| 12 | 49418437 | 49418441 | p1.(G)5 |
| 12 | 49420204 | 49420209 | p1.(C)6 |
| 12 | 49420846 | 49420850 | p1.(G)5 |
| 12 | 49420963 | 49420967 | p1.(G)5 |
| 12 | 49420989 | 49420993 | p1.(G)5 |
| 12 | 49420996 | 49421000 | p1.(G)5 |
| 12 | 49421573 | 49421577 | p1.(C)5 |
| 12 | 49421807 | 49421811 | p1.(C)5 |
| 12 | 49422868 | 49422872 | p1.(G)5 |
| 12 | 49422946 | 49422950 | p1.(C)5 |
| 12 | 49424114 | 49424118 | p1.(C)5 |
| 12 | 49424167 | 49424171 | p1.(G)5 |
| 12 | 49424178 | 49424183 | p1.(G)6 |
| 12 | 49424374 | 49424378 | p1.(T)5 |
| 12 | 49424443 | 49424448 | p1.(C)6 |
| 12 | 49424489 | 49424493 | p1.(G)5 |
| 12 | 49424666 | 49424671 | p1.(G)6 |
| 12 | 49424970 | 49424974 | p1.(G)5 |
| 12 | 49425051 | 49425055 | p1.(C)5 |
| 12 | 49425349 | 49425353 | p1.(G)5 |
| 12 | 49425456 | 49425460 | p1.(G)5 |
| 12 | 49425510 | 49425514 | p1.(G)5 |
| 12 | 49425668 | 49425672 | p1.(G)5 |
| 12 | 49425694 | 49425698 | p1.(C)5 |
| 12 | 49425824 | 49425838 | p3.(GCT)5 |
| 12 | 49425865 | 49425869 | p1.(T)5 |
| 12 | 49426022 | 49426026 | p1.(G)5 |
| 12 | 49426670 | 49426681 | p3.(GCT)4 |
| 12 | 49426730 | 49426750 | p3.(GCT)7 |
| 12 | 49426906 | 49426920 | p3.(TGC)5 |
| 12 | 49426973 | 49426978 | p1.(G)6 |
| 12 | 49427027 | 49427031 | p1.(G)5 |
| 12 | 49427047 | 49427058 | p3.(TGC)4 |
| 12 | 49427251 | 49427262 | p3.(TGC)4 |
| 12 | 49427266 | 49427286 | p3.(TGC)7 |
| 12 | 49427395 | 49427399 | p1.(C)5 |
| 12 | 49427506 | 49427510 | p1.(C)5 |
| 12 | 49427665 | 49427679 | p3.(TGC)5 |
| 12 | 49427843 | 49427847 | p1.(C)5 |
| 12 | 49427937 | 49427942 | p1.(T)6 |
| 12 | 49428411 | 49428416 | p1.(C)6 |
| 12 | 49431291 | 49431302 | p3.(TGC)4 |
| 12 | 49431306 | 49431317 | p3.(TGC)4 |
| 12 | 49431545 | 49431549 | p1.(G)5 |
| 12 | 49431722 | 49431726 | p1.(G)5 |
| 12 | 49431834 | 49431838 | p1.(G)5 |
| 12 | 49431874 | 49431879 | p1.(C)6 |
| 12 | 49432030 | 49432034 | p1.(G)5 |
| 12 | 49432236 | 49432240 | p1.(G)5 |
| 12 | 49432347 | 49432351 | p1.(G)5 |
| 12 | 49432399 | 49432403 | p1.(G)5 |
| 12 | 49432420 | 49432424 | p1.(A)5 |
| 12 | 49432464 | 49432468 | p1.(C)5 |
| 12 | 49433113 | 49433117 | p1.(G)5 |
| 12 | 49433233 | 49433237 | p1.(G)5 |
| 12 | 49433407 | 49433414 | p1.(A)8 |
| 12 | 49433773 | 49433777 | p1.(G)5 |
| 12 | 49433904 | 49433908 | p1.(G)5 |
| 12 | 49433960 | 49433964 | p1.(G)5 |
| 12 | 49434005 | 49434010 | p1.(G)6 |
| 12 | 49434074 | 49434079 | p1.(C)6 |
| 12 | 49434082 | 49434086 | p1.(C)5 |
| 12 | 49434129 | 49434133 | p1.(G)5 |
| 12 | 49434247 | 49434251 | p1.(A)5 |
| 12 | 49434354 | 49434358 | p1.(G)5 |
| 12 | 49434378 | 49434382 | p1.(G)5 |
| 12 | 49434408 | 49434412 | p1.(G)5 |
| 12 | 49434492 | 49434496 | p1.(G)5 |
| 12 | 49434562 | 49434567 | p1.(G)6 |
| 12 | 49434648 | 49434652 | p1.(G)5 |
| 12 | 49434726 | 49434730 | p1.(G)5 |
| 12 | 49434759 | 49434763 | p1.(C)5 |
| 12 | 49434851 | 49434855 | p1.(G)5 |
| 12 | 49434924 | 49434928 | p1.(G)5 |
| 12 | 49434940 | 49434944 | p1.(C)5 |
| 12 | 49434959 | 49434964 | p1.(G)6 |
| 12 | 49435157 | 49435161 | p1.(G)5 |
| 12 | 49435187 | 49435191 | p1.(G)5 |
| 12 | 49435199 | 49435204 | p1.(G)6 |
| 12 | 49435230 | 49435234 | p1.(G)5 |
| 12 | 49435324 | 49435328 | p1.(G)5 |
| 12 | 49435706 | 49435710 | p1.(G)5 |
| 12 | 49436020 | 49436024 | p1.(G)5 |
| 12 | 49436029 | 49436033 | p1.(G)5 |
| 12 | 49436102 | 49436106 | p1.(C)5 |
| 12 | 49436666 | 49436670 | p1.(G)5 |
| 12 | 49436954 | 49436958 | p1.(C)5 |
| 12 | 49437515 | 49437519 | p1.(A)5 |
| 12 | 49438036 | 49438040 | p1.(T)5 |
| 12 | 49438211 | 49438216 | p1.(T)6 |
| 12 | 49440431 | 49440436 | p1.(G)6 |
| 12 | 49441816 | 49441821 | p1.(C)6 |
| 12 | 49442512 | 49442523 | p3.(TCC)4 |
| 12 | 49443512 | 49443516 | p1.(C)5 |
| 12 | 49443641 | 49443645 | p1.(C)5 |
| 12 | 49443667 | 49443672 | p1.(C)6 |
| 12 | 49443789 | 49443793 | p1.(G)5 |
| 12 | 49444053 | 49444057 | p1.(G)5 |
| 12 | 49444073 | 49444077 | p1.(C)5 |
| 12 | 49444181 | 49444185 | p1.(C)5 |
| 12 | 49444363 | 49444367 | p1.(G)5 |
| 12 | 49444378 | 49444383 | p1.(G)6 |
| 12 | 49444443 | 49444447 | p1.(G)5 |
| 12 | 49444505 | 49444509 | p1.(C)5 |
| 12 | 49444809 | 49444813 | p1.(G)5 |
| 12 | 49444863 | 49444867 | p1.(G)5 |
| 12 | 49444933 | 49444938 | p1.(G)6 |
| 12 | 49444960 | 49444965 | p1.(G)6 |
| 12 | 49444987 | 49444992 | p1.(G)6 |
| 12 | 49445041 | 49445046 | p1.(G)6 |
| 12 | 49445095 | 49445100 | p1.(G)6 |
| 12 | 49445149 | 49445154 | p1.(G)6 |
| 12 | 49445203 | 49445208 | p1.(G)6 |
| 12 | 49445257 | 49445261 | p1.(G)5 |
| 12 | 49445375 | 49445379 | p1.(G)5 |
| 12 | 49445500 | 49445505 | p1.(G)6 |
| 12 | 49445526 | 49445532 | p1.(G)7 |
| 12 | 49445883 | 49445887 | p1.(G)5 |
| 12 | 49445929 | 49445933 | p1.(A)5 |
| 12 | 49445949 | 49445953 | p1.(G)5 |
| 12 | 49446138 | 49446142 | p1.(G)5 |
| 12 | 49446166 | 49446171 | p1.(G)6 |
| 12 | 49446462 | 49446466 | p1.(G)5 |
| 12 | 49446481 | 49446485 | p1.(G)5 |
| 12 | 49447430 | 49447434 | p1.(G)5 |
| 12 | 49447773 | 49447777 | p1.(C)5 |
| 12 | 49448147 | 49448151 | p1.(C)5 |
| 12 | 49448408 | 49448413 | p1.(C)6 |
| 12 | 49448529 | 49448533 | p1.(C)5 |
| 12 | 51173906 | 51173916 | p1.(T)6(C)5 |
| 12 | 51189684 | 51189689 | p1.(T)6 |
| 12 | 51203227 | 51203234 | p1.(T)8 |
| 12 | 51203239 | 51203243 | p1.(A)5 |
| 12 | 51207780 | 51207784 | p1.(T)5 |
| 12 | 51208184 | 51208188 | p1.(C)5 |
| 12 | 51208215 | 51208219 | p1.(A)5 |
| 12 | 54332816 | 54332821 | p1.(G)6 |
| 12 | 54333021 | 54333026 | p1.(C)6 |
| 12 | 54333066 | 54333070 | p1.(G)5 |
| 12 | 54367186 | 54367190 | p1.(C)5 |
| 12 | 54367227 | 54367231 | p1.(C)5 |
| 12 | 54367371 | 54367375 | p1.(A)5 |
| 12 | 54367423 | 54367427 | p1.(C)5 |
| 12 | 54367530 | 54367535 | p1.(C)5 |
| 12 | 54367559 | 54367563 | p1.(G)5 |
| 12 | 54367575 | 54367579 | p1.(C)5 |
| 12 | 54367702 | 54367706 | p1.(C)5 |
| 12 | 56474039 | 56474043 | p1.(C)5 |
| 12 | 56480326 | 56480330 | p1.(G)5 |
| 12 | 56481363 | 56481368 | p1.(C)6 |
| 12 | 56481662 | 56481666 | p1.(G)5 |
| 12 | 56481794 | 56481799 | p1.(T)6 |
| 12 | 56482392 | 56482396 | p1.(A)5 |
| 12 | 56482422 | 56482426 | p1.(G)5 |
| 12 | 56486818 | 56486823 | p1.(T)6 |
| 12 | 56487278 | 56487282 | p1.(G)5 |
| 12 | 56487586 | 56487590 | p1.(G)5 |
| 12 | 56488248 | 56488252 | p1.(C)5 |
| 12 | 56490962 | 56490967 | p1.(G)6 |
| 12 | 56492628 | 56492632 | p1.(G)5 |
| 12 | 56493620 | 56493629 | p2.(AG)5 |
| 12 | 56493944 | 56493949 | p1.(T)6 |
| 12 | 56494877 | 56494881 | p1.(C)5 |
| 12 | 56495306 | 56495310 | p1.(C)5 |
| 12 | 56495441 | 56495445 | p1.(C)5 |
| 12 | 56495627 | 56495631 | p1.(G)5 |
| 12 | 56495647 | 56495651 | p1.(G)5 |
| 12 | 56495715 | 56495720 | p1.(C)6 |
| 12 | 56495740 | 56495744 | p1.(A)5 |
| 12 | 57107333 | 57107337 | p1.(A)5 |
| 12 | 57910951 | 57910955 | p1.(T)5 |
| 12 | 57911083 | 57911087 | p1.(C)5 |
| 12 | 58143031 | 58143036 | p1.(G)6 |
| 12 | 58143105 | 58143109 | p1.(G)5 |
| 12 | 58145018 | 58145022 | p1.(G)5 |
| 12 | 59267917 | 59267921 | p1.(T)5 |
| 12 | 59268049 | 59268053 | p1.(T)5 |
| 12 | 59268114 | 59268118 | p1.(T)5 |
| 12 | 59268283 | 59268287 | p1.(A)5 |
| 12 | 59270250 | 59270254 | p1.(A)5 |
| 12 | 59271483 | 59271488 | p1.(A)6 |
| 12 | 59271541 | 59271545 | p1.(G)5 |
| 12 | 59271619 | 59271623 | p1.(A)5 |
| 12 | 59272767 | 59272771 | p1.(C)5 |
| 12 | 59272792 | 59272797 | p1.(G)6 |
| 12 | 59272862 | 59272866 | p1.(G)5 |
| 12 | 59274556 | 59274561 | p1.(T)6 |
| 12 | 59274689 | 59274693 | p1.(A)5 |
| 12 | 59276679 | 59276683 | p1.(A)5 |
| 12 | 59277377 | 59277381 | p1.(A)5 |
| 12 | 59279645 | 59279650 | p1.(T)6 |
| 12 | 59279691 | 59279697 | p1.(A)7 |
| 12 | 59282621 | 59282630 | p1.(C)5(A)5 |
| 12 | 59282718 | 59282722 | p1.(T)5 |
| 12 | 65445175 | 65445179 | p1.(T)5 |
| 12 | 65449910 | 65449914 | p1.(A)5 |
| 12 | 65462692 | 65462698 | p1.(A)7 |
| 12 | 65514341 | 65514345 | p1.(T)5 |
| 12 | 65514813 | 65514817 | p1.(G)5 |
| 12 | 66221835 | 66221839 | p1.(A)5 |
| 12 | 66357072 | 66357076 | p1.(G)5 |
| 12 | 69202230 | 69202234 | p1.(C)5 |
| 12 | 69202972 | 69202979 | p1.(T)8 |
| 12 | 69203080 | 69203084 | p1.(T)5 |
| 12 | 69210596 | 69210601 | p1.(T)6 |
| 12 | 69229771 | 69229778 | p1.(T)8 |
| 12 | 69233090 | 69233096 | p1.(C)7 |
| 12 | 69233240 | 69233246 | p1.(A)7 |
| 12 | 69233489 | 69233493 | p1.(A)5 |
| 12 | 71833843 | 71833847 | p1.(C)5 |
| 12 | 71833921 | 71833925 | p1.(G)5 |
| 12 | 71898385 | 71898391 | p1.(C)7 |
| 12 | 71946884 | 71946888 | p1.(C)5 |
| 12 | 71965286 | 71965290 | p1.(T)5 |
| 12 | 71965330 | 71965334 | p1.(T)5 |
| 12 | 71971691 | 71971697 | p1.(T)7 |
| 12 | 71977559 | 71977563 | p1.(C)5 |
| 12 | 71977713 | 71977718 | p1.(T)6 |
| 12 | 71977724 | 71977728 | p1.(T)5 |
| 12 | 71978363 | 71978367 | p1.(A)5 |
| 12 | 92537884 | 92537888 | p1.(T)5 |
| 12 | 92539312 | 92539316 | p1.(G)5 |
| 12 | 92539325 | 92539329 | p1.(G)5 |
| 12 | 111856235 | 111856239 | p1.(C)5 |
| 12 | 111856434 | 111856438 | p1.(C)5 |
| 12 | 111856443 | 111856447 | p1.(C)5 |
| 12 | 111856464 | 111856468 | p1.(C)5 |
| 12 | 111885145 | 111885150 | p1.(G)6 |
| 12 | 111885352 | 111885376 | p5.(TGGGG)5 |
| 12 | 111885901 | 111885905 | p1.(G)5 |
| 12 | 111885939 | 111885944 | p1.(C)6 |
| 12 | 112204866 | 112204870 | p1.(C)5 |
| 12 | 112227714 | 112227718 | p1.(G)5 |
| 12 | 112229119 | 112229123 | p1.(C)5 |
| 12 | 112229927 | 112229933 | p1.(G)7 |
| 12 | 112235938 | 112235942 | p1.(G)5 |
| 12 | 112235960 | 112235964 | p1.(G)5 |
| 12 | 112884072 | 112884076 | p1.(T)5 |
| 12 | 112884149 | 112884154 | p1.(T)6 |
| 12 | 112890982 | 112890992 | p1.(T)6(A)5 |
| 12 | 112891049 | 112891053 | p1.(A)5 |
| 12 | 112910733 | 112910744 | p4.(TTTC)3 |
| 12 | 112910811 | 112910815 | p1.(A)5 |
| 12 | 112910817 | 112910821 | p1.(A)5 |
| 12 | 112910829 | 112910833 | p1.(A)5 |
| 12 | 112915443 | 112915447 | p1.(T)5 |
| 12 | 112915697 | 112915701 | p1.(A)5 |
| 12 | 112924351 | 112924355 | p1.(G)5 |
| 12 | 121416697 | 121416701 | p1.(C)5 |
| 12 | 121416709 | 121416713 | p1.(G)5 |
| 12 | 121416763 | 121416767 | p1.(G)5 |
| 12 | 121432118 | 121432125 | p1.(C)8 |
| 12 | 121432191 | 121432195 | p1.(C)5 |
| 12 | 121434164 | 121434168 | p1.(C)5 |
| 12 | 121434356 | 121434365 | p1.(G)5(C)5 |
| 12 | 121434367 | 121434372 | p1.(C)6 |
| 12 | 121435461 | 121435465 | p1.(C)5 |
| 12 | 121437279 | 121437283 | p1.(C)5 |
| 12 | 121439034 | 121439039 | p1.(G)6 |
| 12 | 121440235 | 121440239 | p1.(C)5 |
| 12 | 122473237 | 122473241 | p1.(A)5 |
| 12 | 122473252 | 122473257 | p1.(A)6 |
| 12 | 122481782 | 122481786 | p1.(C)5 |
| 12 | 122481828 | 122481832 | p1.(C)5 |
| 12 | 122492892 | 122492896 | p1.(G)5 |
| 12 | 133209268 | 133209272 | p1.(C)5 |
| 12 | 133209298 | 133209303 | p1.(C)6 |
| 12 | 133209314 | 133209318 | p1.(G)5 |
| 12 | 133210819 | 133210823 | p1.(A)5 |
| 12 | 133210882 | 133210893 | p3.(TCC)4 |
| 12 | 133210942 | 133210946 | p1.(C)5 |
| 12 | 133218360 | 133218364 | p1.(C)5 |
| 12 | 133219301 | 133219305 | p1.(C)5 |
| 12 | 133219487 | 133219492 | p1.(G)6 |
| 12 | 133220099 | 133220110 | p2.(CA)6 |
| 12 | 133225574 | 133225578 | p1.(G)5 |
| 12 | 133233794 | 133233799 | p1.(T)6 |
| 12 | 133238184 | 133238189 | p1.(A)6 |
| 12 | 133242026 | 133242030 | p1.(T)5 |
| 12 | 133245024 | 133245030 | p1.(G)7 |
| 12 | 133245435 | 133245439 | p1.(C)5 |
| 12 | 133249274 | 133249278 | p1.(C)5 |
| 12 | 133252024 | 133252028 | p1.(C)5 |
| 12 | 133252326 | 133252331 | p1.(A)6 |
| 12 | 133252728 | 133252732 | p1.(G)5 |
| 12 | 133264011 | 133264015 | p1.(C)5 |
| 12 | 133264017 | 133264022 | p1.(C)6 |
| 12 | 133264053 | 133264057 | p1.(G)5 |
| 13 | 20567378 | 20567389 | p3.(GAT)4 |
| 13 | 20567394 | 20567398 | p1.(T)5 |
| 13 | 20567417 | 20567421 | p1.(C)5 |
| 13 | 20567473 | 20567478 | p1.(A)6 |
| 13 | 20567609 | 20567613 | p1.(A)5 |
| 13 | 20567651 | 20567655 | p1.(A)5 |
| 13 | 20567689 | 20567693 | p1.(T)5 |
| 13 | 20568021 | 20568025 | p1.(T)5 |
| 13 | 20576982 | 20576986 | p1.(T)5 |
| 13 | 20577145 | 20577150 | p1.(A)6 |
| 13 | 20577271 | 20577275 | p1.(A)5 |
| 13 | 20580502 | 20580506 | p1.(T)5 |
| 13 | 20580535 | 20580539 | p1.(A)5 |
| 13 | 20593678 | 20593684 | p1.(T)7 |
| 13 | 20605534 | 20605538 | p1.(A)5 |
| 13 | 20610869 | 20610873 | p1.(T)5 |
| 13 | 20611023 | 20611028 | p1.(A)6 |
| 13 | 20625560 | 20625564 | p1.(T)5 |
| 13 | 20632727 | 20632731 | p1.(C)5 |
| 13 | 20633580 | 20633584 | p1.(T)5 |
| 13 | 20635206 | 20635210 | p1.(T)5 |
| 13 | 20635264 | 20635268 | p1.(A)5 |
| 13 | 20640977 | 20640982 | p1.(T)6 |
| 13 | 20641372 | 20641376 | p1.(T)5 |
| 13 | 20657094 | 20657101 | p1.(A)8 |
| 13 | 20657787 | 20657792 | p1.(T)6 |
| 13 | 20657812 | 20657816 | p1.(A)5 |
| 13 | 20659991 | 20659996 | p1.(T)6 |
| 13 | 20660153 | 20660157 | p1.(A)5 |
| 13 | 26828733 | 26828738 | p1.(C)6 |
| 13 | 26828741 | 26828746 | p1.(C)6 |
| 13 | 26828911 | 26828920 | p2.(GT)5 |
| 13 | 26927863 | 26927874 | p4.(TTTC)3 |
| 13 | 26975408 | 26975412 | p1.(T)5 |
| 13 | 26975446 | 26975450 | p1.(T)5 |
| 13 | 26975609 | 26975623 | p3.(CAG)5 |
| 13 | 26975687 | 26975691 | p1.(C)5 |
| 13 | 28537278 | 28537284 | p1.(C)7 |
| 13 | 28537425 | 28537436 | p3.(GCT)4 |
| 13 | 28543040 | 28543045 | p1.(G)6 |
| 13 | 28588620 | 28588624 | p1.(A)5 |
| 13 | 28589809 | 28589813 | p1.(G)5 |
| 13 | 28597588 | 28597593 | p1.(T)6 |
| 13 | 28599086 | 28599091 | p1.(A)6 |
| 13 | 28601256 | 28601260 | p1.(A)5 |
| 13 | 28601305 | 28601309 | p1.(T)5 |
| 13 | 28601359 | 28601363 | p1.(A)5 |
| 13 | 28602330 | 28602334 | p1.(C)5 |
| 13 | 28602421 | 28602425 | p1.(T)5 |
| 13 | 28608439 | 28608443 | p1.(T)5 |
| 13 | 28609814 | 28609823 | p1.(A)10 |
| 13 | 28611433 | 28611437 | p1.(A)5 |
| 13 | 28622509 | 28622513 | p1.(A)5 |
| 13 | 28622588 | 28622593 | p1.(A)6 |
| 13 | 28624317 | 28624321 | p1.(T)5 |
| 13 | 28624327 | 28624331 | p1.(T)5 |
| 13 | 28626686 | 28626690 | p1.(C)5 |
| 13 | 28626770 | 28626774 | p1.(T)5 |
| 13 | 28631536 | 28631540 | p1.(A)5 |
| 13 | 28644741 | 28644746 | p1.(A)6 |
| 13 | 28877347 | 28877351 | p1.(G)5 |
| 13 | 28877508 | 28877519 | p4.(AAAG)3 |
| 13 | 28880913 | 28880917 | p1.(G)5 |
| 13 | 28886163 | 28886167 | p1.(T)5 |
| 13 | 28897047 | 28897052 | p1.(T)6 |
| 13 | 28901608 | 28901613 | p1.(A)6 |
| 13 | 28903818 | 28903822 | p1.(T)5 |
| 13 | 28903860 | 28903864 | p1.(C)5 |
| 13 | 28959048 | 28959052 | p1.(T)5 |
| 13 | 28964077 | 28964082 | p1.(T)6 |
| 13 | 28964206 | 28964211 | p1.(T)6 |
| 13 | 28980037 | 28980046 | p1.(A)10 |
| 13 | 29001915 | 29001919 | p1.(T)5 |
| 13 | 29002067 | 29002071 | p1.(A)5 |
| 13 | 29005443 | 29005447 | p1.(T)5 |
| 13 | 29012359 | 29012364 | p1.(T)6 |
| 13 | 29041262 | 29041267 | p1.(C)6 |
| 13 | 32890594 | 32890598 | p1.(A)5 |
| 13 | 32890628 | 32890633 | p1.(T)6 |
| 13 | 32890638 | 32890642 | p1.(T)5 |
| 13 | 32900296 | 32900300 | p1.(A)5 |
| 13 | 32900364 | 32900370 | p1.(T)7 |
| 13 | 32900372 | 32900376 | p1.(C)5 |
| 13 | 32905047 | 32905051 | p1.(T)5 |
| 13 | 32905070 | 32905074 | p1.(T)5 |
| 13 | 32905098 | 32905102 | p1.(A)5 |
| 13 | 32906416 | 32906420 | p1.(A)5 |
| 13 | 32906536 | 32906540 | p1.(T)5 |
| 13 | 32906548 | 32906552 | p1.(T)5 |
| 13 | 32906566 | 32906571 | p1.(A)6 |
| 13 | 32906577 | 32906581 | p1.(A)5 |
| 13 | 32906603 | 32906609 | p1.(A)7 |
| 13 | 32906640 | 32906644 | p1.(A)5 |
| 13 | 32906648 | 32906652 | p1.(A)5 |
| 13 | 32906664 | 32906668 | p1.(A)5 |
| 13 | 32906889 | 32906893 | p1.(A)5 |
| 13 | 32906916 | 32906927 | p4.(AAAG)3 |
| 13 | 32907119 | 32907123 | p1.(A)5 |
| 13 | 32907172 | 32907176 | p1.(T)5 |
| 13 | 32907203 | 32907208 | p1.(A)6 |
| 13 | 32907365 | 32907369 | p1.(A)5 |
| 13 | 32907421 | 32907428 | p1.(A)8 |
| 13 | 32907441 | 32907445 | p1.(A)5 |
| 13 | 32910389 | 32910393 | p1.(T)5 |
| 13 | 32910579 | 32910583 | p1.(A)5 |
| 13 | 32910655 | 32910659 | p1.(A)5 |
| 13 | 32910662 | 32910667 | p1.(A)6 |
| 13 | 32910923 | 32910927 | p1.(A)5 |
| 13 | 32910977 | 32910981 | p1.(A)5 |
| 13 | 32911002 | 32911006 | p1.(A)5 |
| 13 | 32911074 | 32911080 | p1.(A)7 |
| 13 | 32911105 | 32911109 | p1.(A)5 |
| 13 | 32911322 | 32911327 | p1.(A)6 |
| 13 | 32911358 | 32911362 | p1.(A)5 |
| 13 | 32911381 | 32911385 | p1.(A)5 |
| 13 | 32911443 | 32911449 | p1.(A)7 |
| 13 | 32911736 | 32911740 | p1.(A)5 |
| 13 | 32912081 | 32912085 | p1.(A)5 |
| 13 | 32912196 | 32912200 | p1.(A)5 |
| 13 | 32912346 | 32912352 | p1.(A)7 |
| 13 | 32912519 | 32912523 | p1.(A)5 |
| 13 | 32912656 | 32912661 | p1.(T)6 |
| 13 | 32912771 | 32912776 | p1.(T)6 |
| 13 | 32912792 | 32912796 | p1.(A)5 |
| 13 | 32913080 | 32913085 | p1.(A)6 |
| 13 | 32913119 | 32913123 | p1.(A)5 |
| 13 | 32913126 | 32913130 | p1.(T)5 |
| 13 | 32913135 | 32913139 | p1.(A)5 |
| 13 | 32913296 | 32913300 | p1.(A)5 |
| 13 | 32913382 | 32913386 | p1.(A)5 |
| 13 | 32913392 | 32913396 | p1.(T)5 |
| 13 | 32913423 | 32913427 | p1.(A)5 |
| 13 | 32913436 | 32913440 | p1.(A)5 |
| 13 | 32913502 | 32913506 | p1.(T)5 |
| 13 | 32913523 | 32913527 | p1.(A)5 |
| 13 | 32913559 | 32913565 | p1.(A)7 |
| 13 | 32913677 | 32913681 | p1.(A)5 |
| 13 | 32913693 | 32913697 | p1.(A)5 |
| 13 | 32913784 | 32913789 | p1.(A)6 |
| 13 | 32913837 | 32913843 | p1.(A)7 |
| 13 | 32913850 | 32913854 | p1.(T)5 |
| 13 | 32913953 | 32913957 | p1.(A)5 |
| 13 | 32913959 | 32913963 | p1.(A)5 |
| 13 | 32914070 | 32914075 | p1.(A)6 |
| 13 | 32914138 | 32914142 | p1.(A)5 |
| 13 | 32914251 | 32914255 | p1.(T)5 |
| 13 | 32914422 | 32914426 | p1.(T)5 |
| 13 | 32914617 | 32914621 | p1.(A)5 |
| 13 | 32914801 | 32914805 | p1.(A)5 |
| 13 | 32914860 | 32914865 | p1.(A)6 |
| 13 | 32915054 | 32915058 | p1.(A)5 |
| 13 | 32915062 | 32915066 | p1.(A)5 |
| 13 | 32915089 | 32915093 | p1.(T)5 |
| 13 | 32915250 | 32915254 | p1.(T)5 |
| 13 | 32915302 | 32915306 | p1.(A)5 |
| 13 | 32929096 | 32929100 | p1.(A)5 |
| 13 | 32929162 | 32929167 | p1.(A)6 |
| 13 | 32929326 | 32929330 | p1.(A)5 |
| 13 | 32929365 | 32929369 | p1.(A)5 |
| 13 | 32930668 | 32930672 | p1.(A)5 |
| 13 | 32931911 | 32931915 | p1.(A)5 |
| 13 | 32931924 | 32931928 | p1.(A)5 |
| 13 | 32937355 | 32937360 | p1.(A)6 |
| 13 | 32937388 | 32937392 | p1.(A)5 |
| 13 | 32937480 | 32937484 | p1.(A)5 |
| 13 | 32953633 | 32953639 | p1.(A)7 |
| 13 | 32953641 | 32953645 | p1.(A)5 |
| 13 | 32954023 | 32954030 | p1.(A)8 |
| 13 | 32954204 | 32954208 | p1.(T)5 |
| 13 | 32954273 | 32954279 | p1.(A)7 |
| 13 | 32968995 | 32968999 | p1.(T)5 |
| 13 | 32969056 | 32969060 | p1.(A)5 |
| 13 | 32972287 | 32972293 | p1.(T)7 |
| 13 | 32972446 | 32972450 | p1.(A)5 |
| 13 | 32972590 | 32972595 | p1.(A)6 |
| 13 | 32972626 | 32972631 | p1.(A)6 |
| 13 | 32972726 | 32972730 | p1.(A)5 |
| 13 | 32972866 | 32972870 | p1.(A)5 |
| 13 | 32972893 | 32972898 | p1.(A)6 |
| 13 | 40174968 | 40174972 | p1.(C)5 |
| 13 | 40175061 | 40175072 | p3.(AGG)4 |
| 13 | 41133898 | 41133903 | p1.(C)6 |
| 13 | 41134359 | 41134363 | p1.(T)5 |
| 13 | 41134928 | 41134932 | p1.(T)5 |
| 13 | 41239933 | 41239938 | p1.(G)6 |
| 13 | 41240355 | 41240359 | p1.(C)5 |
| 13 | 46701833 | 46701837 | p1.(T)5 |
| 13 | 46721052 | 46721057 | p1.(C)6 |
| 13 | 46728965 | 46728969 | p1.(T)5 |
| 13 | 46732768 | 46732772 | p1.(T)5 |
| 13 | 46732791 | 46732795 | p1.(A)5 |
| 13 | 46733797 | 46733802 | p1.(T)6 |
| 13 | 48878062 | 48878067 | p1.(C)6 |
| 13 | 48878069 | 48878073 | p1.(A)5 |
| 13 | 48878106 | 48878110 | p1.(C)5 |
| 13 | 48878115 | 48878126 | p3.(CCG)4 |
| 13 | 48878127 | 48878131 | p1.(C)5 |
| 13 | 48881489 | 48881498 | p2.(AG)5 |
| 13 | 48916721 | 48916726 | p1.(T)6 |
| 13 | 48916753 | 48916757 | p1.(A)5 |
| 13 | 48916834 | 48916838 | p1.(A)5 |
| 13 | 48934141 | 48934149 | p1.(T)9 |
| 13 | 48941616 | 48941621 | p1.(T)6 |
| 13 | 48941669 | 48941673 | p1.(A)5 |
| 13 | 48941696 | 48941700 | p1.(T)5 |
| 13 | 48942687 | 48942691 | p1.(A)5 |
| 13 | 49030368 | 49030373 | p1.(A)6 |
| 13 | 49030479 | 49030484 | p1.(A)6 |
| 13 | 49039329 | 49039333 | p1.(T)5 |
| 13 | 49039341 | 49039345 | p1.(C)5 |
| 13 | 49039484 | 49039488 | p1.(A)5 |
| 13 | 49050933 | 49050938 | p1.(A)6 |
| 13 | 49054125 | 49054129 | p1.(T)5 |
| 13 | 103053835 | 103053839 | p1.(T)5 |
| 13 | 103054032 | 103054036 | p1.(A)5 |
| 13 | 103498619 | 103498623 | p1.(G)5 |
| 13 | 103504578 | 103504583 | p1.(T)6 |
| 13 | 103504597 | 103504601 | p1.(T)5 |
| 13 | 103506715 | 103506719 | p1.(A)5 |
| 13 | 103508419 | 103508423 | p1.(A)5 |
| 13 | 103508446 | 103508450 | p1.(A)5 |
| 13 | 103510685 | 103510689 | p1.(C)5 |
| 13 | 103513899 | 103513903 | p1.(A)5 |
| 13 | 103513992 | 103513996 | p1.(G)5 |
| 13 | 103514479 | 103514483 | p1.(A)5 |
| 13 | 103518670 | 103518674 | p1.(A)5 |
| 13 | 103519135 | 103519140 | p1.(T)6 |
| 13 | 103519144 | 103519148 | p1.(A)5 |
| 13 | 103524568 | 103524574 | p1.(A)7 |
| 13 | 103524612 | 103524620 | p1.(A)9 |
| 13 | 103524718 | 103524722 | p1.(G)5 |
| 13 | 103527648 | 103527654 | p1.(T)7 |
| 13 | 103527851 | 103527855 | p1.(A)5 |
| 13 | 103527951 | 103527960 | p1.(T)5(G)5 |
| 13 | 103528058 | 103528062 | p1.(A)5 |
| 13 | 103528238 | 103528242 | p1.(A)5 |
| 13 | 103528256 | 103528261 | p1.(A)6 |
| 13 | 110434454 | 110434458 | p1.(G)5 |
| 13 | 110434477 | 110434482 | p1.(C)6 |
| 13 | 110434526 | 110434530 | p1.(C)5 |
| 13 | 110434578 | 110434592 | p3.(GCG)5 |
| 13 | 110434721 | 110434725 | p1.(C)5 |
| 13 | 110434766 | 110434770 | p1.(G)5 |
| 13 | 110434811 | 110434815 | p1.(C)5 |
| 13 | 110434921 | 110434925 | p1.(G)5 |
| 13 | 110434962 | 110434973 | p3.(GGC)4 |
| 13 | 110434973 | 110434978 | p1.(C)6 |
| 13 | 110435013 | 110435017 | p1.(G)5 |
| 13 | 110435021 | 110435025 | p1.(G)5 |
| 13 | 110435129 | 110435135 | p1.(G)7 |
| 13 | 110435263 | 110435268 | p1.(G)6 |
| 13 | 110435283 | 110435287 | p1.(C)5 |
| 13 | 110435345 | 110435349 | p1.(G)5 |
| 13 | 110435377 | 110435381 | p1.(G)5 |
| 13 | 110435443 | 110435447 | p1.(G)5 |
| 13 | 110435816 | 110435820 | p1.(G)5 |
| 13 | 110435914 | 110435918 | p1.(G)5 |
| 13 | 110435962 | 110435966 | p1.(G)5 |
| 13 | 110436088 | 110436092 | p1.(G)5 |
| 13 | 110436190 | 110436194 | p1.(G)5 |
| 13 | 110436227 | 110436231 | p1.(C)5 |
| 13 | 110436297 | 110436320 | p3.(CGG)8 |
| 13 | 110436343 | 110436347 | p1.(G)5 |
| 13 | 110436456 | 110436460 | p1.(C)5 |
| 13 | 110436717 | 110436721 | p1.(C)5 |
| 13 | 110436791 | 110436805 | p3.(CCG)5 |
| 13 | 110436815 | 110436819 | p1.(G)5 |
| 13 | 110436925 | 110436929 | p1.(G)5 |
| 13 | 110437133 | 110437137 | p1.(C)5 |
| 13 | 110437233 | 110437237 | p1.(G)5 |
| 13 | 110437402 | 110437407 | p1.(G)6 |
| 13 | 110437755 | 110437759 | p1.(C)5 |
| 13 | 110438159 | 110438163 | p1.(T)5 |
| 14 | 20779717 | 20779721 | p1.(T)5 |
| 14 | 20779784 | 20779789 | p1.(A)6 |
| 14 | 20779826 | 20779831 | p1.(A)6 |
| 14 | 20781791 | 20781795 | p1.(T)5 |
| 14 | 23777233 | 23777237 | p1.(T)5 |
| 14 | 23777241 | 23777245 | p1.(G)5 |
| 14 | 23777400 | 23777404 | p1.(G)5 |
| 14 | 23778117 | 23778121 | p1.(G)5 |
| 14 | 23778132 | 23778136 | p1.(G)5 |
| 14 | 23778157 | 23778162 | p1.(T)6 |
| 14 | 23778207 | 23778211 | p1.(G)5 |
| 14 | 35873802 | 35873806 | p1.(G)5 |
| 14 | 35873826 | 35873830 | p1.(G)5 |
| 14 | 36986877 | 36986881 | p1.(C)5 |
| 14 | 36986886 | 36986890 | p1.(C)5 |
| 14 | 36986889 | 36986900 | p3.(CCG)4 |
| 14 | 36987157 | 36987161 | p1.(C)5 |
| 14 | 36988309 | 36988314 | p1.(C)6 |
| 14 | 36988340 | 36988344 | p1.(C)5 |
| 14 | 36988599 | 36988606 | p1.(A)8 |
| 14 | 38060553 | 38060558 | p1.(C)6 |
| 14 | 38060812 | 38060816 | p1.(G)5 |
| 14 | 38060912 | 38060917 | p1.(G)6 |
| 14 | 38060955 | 38060959 | p1.(C)5 |
| 14 | 38060992 | 38060996 | p1.(G)5 |
| 14 | 38061011 | 38061015 | p1.(G)5 |
| 14 | 38061138 | 38061142 | p1.(C)5 |
| 14 | 38061156 | 38061160 | p1.(C)5 |
| 14 | 38061169 | 38061173 | p1.(C)5 |
| 14 | 38061517 | 38061528 | p3.(CGC)4 |
| 14 | 38061726 | 38061731 | p1.(C)6 |
| 14 | 51202268 | 51202272 | p1.(T)5 |
| 14 | 51202342 | 51202349 | p1.(A)8 |
| 14 | 51204848 | 51204852 | p1.(A)5 |
| 14 | 51204918 | 51204922 | p1.(T)5 |
| 14 | 51204963 | 51204967 | p1.(T)5 |
| 14 | 51211015 | 51211019 | p1.(T)5 |
| 14 | 51211044 | 51211049 | p1.(T)6 |
| 14 | 51214831 | 51214835 | p1.(A)5 |
| 14 | 51219317 | 51219321 | p1.(T)5 |
| 14 | 51219368 | 51219372 | p1.(T)5 |
| 14 | 51219429 | 51219433 | p1.(T)5 |
| 14 | 51219436 | 51219440 | p1.(T)5 |
| 14 | 51223224 | 51223228 | p1.(T)5 |
| 14 | 51223311 | 51223315 | p1.(T)5 |
| 14 | 51223539 | 51223543 | p1.(T)5 |
| 14 | 51224050 | 51224055 | p1.(T)6 |
| 14 | 51224070 | 51224074 | p1.(A)5 |
| 14 | 51224207 | 51224211 | p1.(A)5 |
| 14 | 51224368 | 51224372 | p1.(A)5 |
| 14 | 51224400 | 51224405 | p1.(A)6 |
| 14 | 51225062 | 51225071 | p2.(TC)5 |
| 14 | 51225341 | 51225346 | p1.(T)6 |
| 14 | 51226868 | 51226873 | p1.(T)6 |
| 14 | 51226953 | 51226957 | p1.(T)5 |
| 14 | 51226973 | 51226977 | p1.(T)5 |
| 14 | 51237150 | 51237154 | p1.(T)5 |
| 14 | 51237214 | 51237223 | p2.(CT)5 |
| 14 | 51237236 | 51237241 | p1.(T)6 |
| 14 | 51237680 | 51237684 | p1.(T)5 |
| 14 | 51239659 | 51239664 | p1.(T)6 |
| 14 | 51239684 | 51239688 | p1.(T)5 |
| 14 | 51239734 | 51239738 | p1.(T)5 |
| 14 | 51239752 | 51239757 | p1.(A)6 |
| 14 | 56078944 | 56078948 | p1.(A)5 |
| 14 | 56078953 | 56078957 | p1.(A)5 |
| 14 | 56079108 | 56079113 | p1.(A)6 |
| 14 | 56079187 | 56079192 | p1.(A)6 |
| 14 | 56079277 | 56079281 | p1.(A)5 |
| 14 | 56084825 | 56084830 | p1.(A)6 |
| 14 | 56096688 | 56096699 | p4.(AGAA)3 |
| 14 | 56113679 | 56113683 | p1.(T)5 |
| 14 | 56113719 | 56113723 | p1.(A)5 |
| 14 | 56114751 | 56114755 | p1.(A)5 |
| 14 | 56115514 | 56115518 | p1.(A)5 |
| 14 | 56119727 | 56119731 | p1.(T)5 |
| 14 | 56122748 | 56122753 | p1.(A)6 |
| 14 | 56126396 | 56126400 | p1.(C)5 |
| 14 | 56130661 | 56130665 | p1.(A)5 |
| 14 | 56130685 | 56130689 | p1.(A)5 |
| 14 | 56130722 | 56130727 | p1.(A)6 |
| 14 | 56137475 | 56137481 | p1.(A)7 |
| 14 | 56138345 | 56138349 | p1.(A)5 |
| 14 | 56142544 | 56142548 | p1.(T)5 |
| 14 | 56145099 | 56145103 | p1.(A)5 |
| 14 | 56146276 | 56146280 | p1.(T)5 |
| 14 | 56150851 | 56150856 | p1.(A)6 |
| 14 | 65543166 | 65543170 | p1.(T)5 |
| 14 | 65543267 | 65543271 | p1.(C)5 |
| 14 | 65544693 | 65544697 | p1.(T)5 |
| 14 | 66975316 | 66975320 | p1.(G)5 |
| 14 | 67389497 | 67389501 | p1.(C)5 |
| 14 | 67389526 | 67389530 | p1.(C)5 |
| 14 | 67555750 | 67555755 | p1.(C)6 |
| 14 | 67579894 | 67579898 | p1.(A)5 |
| 14 | 67610068 | 67610074 | p1.(T)7 |
| 14 | 67610143 | 67610147 | p1.(G)5 |
| 14 | 67610157 | 67610161 | p1.(G)5 |
| 14 | 67626190 | 67626194 | p1.(T)5 |
| 14 | 67631865 | 67631871 | p1.(T)7 |
| 14 | 67631921 | 67631925 | p1.(A)5 |
| 14 | 68292184 | 68292188 | p1.(T)5 |
| 14 | 68301921 | 68301930 | p1.(A)5(T)5 |
| 14 | 81554355 | 81554359 | p1.(C)5 |
| 14 | 81609362 | 81609366 | p1.(C)5 |
| 14 | 81609513 | 81609522 | p1.(A)5(C)5 |
| 14 | 81609598 | 81609602 | p1.(C)5 |
| 14 | 81609746 | 81609750 | p1.(C)5 |
| 14 | 81610032 | 81610036 | p1.(G)5 |
| 14 | 81610153 | 81610157 | p1.(T)5 |
| 14 | 81610522 | 81610526 | p1.(G)5 |
| 14 | 92436189 | 92436193 | p1.(A)5 |
| 14 | 92441021 | 92441025 | p1.(T)5 |
| 14 | 92441583 | 92441587 | p1.(T)5 |
| 14 | 92442441 | 92442445 | p1.(A)5 |
| 14 | 92460190 | 92460194 | p1.(T)5 |
| 14 | 92460216 | 92460220 | p1.(T)5 |
| 14 | 92460249 | 92460253 | p1.(T)5 |
| 14 | 92461868 | 92461872 | p1.(A)5 |
| 14 | 92465574 | 92465579 | p1.(A)6 |
| 14 | 92466342 | 92466346 | p1.(T)5 |
| 14 | 92469832 | 92469836 | p1.(T)5 |
| 14 | 92469918 | 92469923 | p1.(T)6 |
| 14 | 92470084 | 92470088 | p1.(T)5 |
| 14 | 92470243 | 92470247 | p1.(T)5 |
| 14 | 92470472 | 92470476 | p1.(T)5 |
| 14 | 92470813 | 92470817 | p1.(T)5 |
| 14 | 92470894 | 92470899 | p1.(T)6 |
| 14 | 92470971 | 92470975 | p1.(T)5 |
| 14 | 92471338 | 92471342 | p1.(A)5 |
| 14 | 92471409 | 92471414 | p1.(T)6 |
| 14 | 92471444 | 92471448 | p1.(A)5 |
| 14 | 92471767 | 92471771 | p1.(T)5 |
| 14 | 92471798 | 92471803 | p1.(T)6 |
| 14 | 92471872 | 92471876 | p1.(A)5 |
| 14 | 92471981 | 92471985 | p1.(T)5 |
| 14 | 92472131 | 92472135 | p1.(T)5 |
| 14 | 92472166 | 92472170 | p1.(T)5 |
| 14 | 92472174 | 92472178 | p1.(T)5 |
| 14 | 92472197 | 92472203 | p1.(T)7 |
| 14 | 92472698 | 92472704 | p1.(T)7 |
| 14 | 92472800 | 92472804 | p1.(A)5 |
| 14 | 92477357 | 92477361 | p1.(T)5 |
| 14 | 92477421 | 92477425 | p1.(A)5 |
| 14 | 92482209 | 92482214 | p1.(A)6 |
| 14 | 92487883 | 92487893 | p1.(A)6(T)5 |
| 14 | 92491657 | 92491661 | p1.(T)5 |
| 14 | 92491769 | 92491777 | p1.(A)9 |
| 14 | 92505968 | 92505972 | p1.(C)5 |
| 14 | 92506010 | 92506014 | p1.(C)5 |
| 14 | 92506065 | 92506070 | p1.(A)6 |
| 14 | 93263894 | 93263898 | p1.(A)5 |
| 14 | 93264000 | 93264004 | p1.(A)5 |
| 14 | 93264121 | 93264126 | p1.(A)6 |
| 14 | 93264210 | 93264214 | p1.(A)5 |
| 14 | 93273069 | 93273076 | p1.(T)8 |
| 14 | 93275634 | 93275639 | p1.(T)6 |
| 14 | 93275855 | 93275859 | p1.(A)5 |
| 14 | 93282603 | 93282607 | p1.(A)5 |
| 14 | 93286040 | 93286044 | p1.(T)5 |
| 14 | 93299699 | 93299704 | p1.(A)6 |
| 14 | 93301931 | 93301935 | p1.(T)5 |
| 14 | 93303745 | 93303749 | p1.(T)5 |
| 14 | 93303775 | 93303779 | p1.(T)5 |
| 14 | 93305824 | 93305828 | p1.(T)5 |
| 14 | 95556792 | 95556799 | p1.(T)8 |
| 14 | 95556824 | 95556828 | p1.(A)5 |
| 14 | 95557005 | 95557010 | p1.(A)6 |
| 14 | 95557359 | 95557364 | p1.(T)6 |
| 14 | 95557631 | 95557635 | p1.(A)5 |
| 14 | 95557639 | 95557643 | p1.(C)5 |
| 14 | 95560398 | 95560402 | p1.(C)5 |
| 14 | 95562799 | 95562804 | p1.(T)6 |
| 14 | 95562840 | 95562844 | p1.(A)5 |
| 14 | 95562982 | 95562993 | p3.(CTC)4 |
| 14 | 95569684 | 95569688 | p1.(T)5 |
| 14 | 95569765 | 95569769 | p1.(A)5 |
| 14 | 95570433 | 95570438 | p1.(T)6 |
| 14 | 95572563 | 95572568 | p1.(A)6 |
| 14 | 95573983 | 95573987 | p1.(A)5 |
| 14 | 95574205 | 95574211 | p1.(T)7 |
| 14 | 95574281 | 95574285 | p1.(T)5 |
| 14 | 95579570 | 95579574 | p1.(A)5 |
| 14 | 95590568 | 95590572 | p1.(A)5 |
| 14 | 95590628 | 95590632 | p1.(T)5 |
| 14 | 95590875 | 95590879 | p1.(A)5 |
| 14 | 95591010 | 95591017 | p1.(A)8 |
| 14 | 95598841 | 95598846 | p1.(T)6 |
| 14 | 95598876 | 95598880 | p1.(T)5 |
| 14 | 95599820 | 95599824 | p1.(T)5 |
| 14 | 96178662 | 96178666 | p1.(G)5 |
| 14 | 99640549 | 99640553 | p1.(T)5 |
| 14 | 99641206 | 99641210 | p1.(C)5 |
| 14 | 99641679 | 99641683 | p1.(G)5 |
| 14 | 99641914 | 99641918 | p1.(G)5 |
| 14 | 99641932 | 99641936 | p1.(G)5 |
| 14 | 99642214 | 99642218 | p1.(G)5 |
| 14 | 99697775 | 99697779 | p1.(G)5 |
| 14 | 99697857 | 99697861 | p1.(G)5 |
| 14 | 99697901 | 99697906 | p1.(A)6 |
| 14 | 99723872 | 99723876 | p1.(G)5 |
| 14 | 99724016 | 99724020 | p1.(A)5 |
| 14 | 99724030 | 99724034 | p1.(C)5 |
| 14 | 102548790 | 102548794 | p1.(A)5 |
| 14 | 102549380 | 102549385 | p1.(T)6 |
| 14 | 102549430 | 102549436 | p1.(T)7 |
| 14 | 102549450 | 102549454 | p1.(T)5 |
| 14 | 102550137 | 102550141 | p1.(T)5 |
| 14 | 102550211 | 102550216 | p1.(T)6 |
| 14 | 102550324 | 102550330 | p1.(A)7 |
| 14 | 102550810 | 102550814 | p1.(T)5 |
| 14 | 102550895 | 102550899 | p1.(A)5 |
| 14 | 102551161 | 102551178 | p3.(TCT)6 |
| 14 | 102551258 | 102551262 | p1.(T)5 |
| 14 | 102551264 | 102551268 | p1.(T)5 |
| 14 | 102551625 | 102551629 | p1.(A)5 |
| 14 | 102551635 | 102551639 | p1.(A)5 |
| 14 | 102552121 | 102552125 | p1.(C)5 |
| 14 | 102552470 | 102552475 | p1.(A)6 |
| 14 | 102605734 | 102605738 | p1.(G)5 |
| 14 | 105236710 | 105236714 | p1.(G)5 |
| 14 | 105238778 | 105238782 | p1.(C)5 |
| 14 | 105239313 | 105239317 | p1.(A)5 |
| 14 | 105239385 | 105239389 | p1.(C)5 |
| 14 | 105239591 | 105239595 | p1.(G)5 |
| 14 | 105240253 | 105240257 | p1.(C)5 |
| 14 | 105242073 | 105242084 | p3.(CTC)4 |
| 15 | 34640379 | 34640383 | p1.(G)5 |
| 15 | 34640439 | 34640444 | p1.(G)6 |
| 15 | 34640680 | 34640684 | p1.(A)5 |
| 15 | 34642894 | 34642898 | p1.(G)5 |
| 15 | 34646683 | 34646688 | p1.(C)6 |
| 15 | 34647271 | 34647275 | p1.(A)5 |
| 15 | 34647838 | 34647842 | p1.(G)5 |
| 15 | 34647845 | 34647849 | p1.(G)5 |
| 15 | 34647969 | 34647973 | p1.(C)5 |
| 15 | 34648286 | 34648290 | p1.(G)5 |
| 15 | 34648377 | 34648381 | p1.(C)5 |
| 15 | 34649080 | 34649084 | p1.(C)5 |
| 15 | 34649123 | 34649127 | p1.(A)5 |
| 15 | 34649306 | 34649310 | p1.(A)5 |
| 15 | 34649312 | 34649316 | p1.(A)5 |
| 15 | 40462330 | 40462334 | p1.(T)5 |
| 15 | 40462833 | 40462837 | p1.(A)5 |
| 15 | 40475980 | 40475984 | p1.(T)5 |
| 15 | 40476036 | 40476040 | p1.(A)5 |
| 15 | 40477353 | 40477357 | p1.(T)5 |
| 15 | 40477435 | 40477439 | p1.(T)5 |
| 15 | 40477506 | 40477511 | p1.(C)6 |
| 15 | 40494781 | 40494785 | p1.(T)5 |
| 15 | 40494803 | 40494807 | p1.(C)5 |
| 15 | 40504777 | 40504781 | p1.(T)5 |
| 15 | 40509789 | 40509793 | p1.(T)5 |
| 15 | 40512758 | 40512762 | p1.(T)5 |
| 15 | 40512872 | 40512876 | p1.(T)5 |
| 15 | 40912893 | 40912898 | p1.(T)6 |
| 15 | 40913048 | 40913053 | p1.(A)6 |
| 15 | 40913062 | 40913066 | p1.(T)5 |
| 15 | 40913119 | 40913123 | p1.(A)5 |
| 15 | 40913289 | 40913293 | p1.(A)5 |
| 15 | 40913467 | 40913471 | p1.(T)5 |
| 15 | 40913550 | 40913554 | p1.(T)5 |
| 15 | 40913635 | 40913639 | p1.(G)5 |
| 15 | 40913810 | 40913814 | p1.(A)5 |
| 15 | 40913955 | 40913959 | p1.(T)5 |
| 15 | 40914006 | 40914010 | p1.(A)5 |
| 15 | 40914353 | 40914357 | p1.(A)5 |
| 15 | 40914593 | 40914597 | p1.(A)5 |
| 15 | 40914879 | 40914883 | p1.(A)5 |
| 15 | 40914903 | 40914908 | p1.(A)6 |
| 15 | 40915030 | 40915034 | p1.(A)5 |
| 15 | 40915589 | 40915593 | p1.(A)5 |
| 15 | 40915657 | 40915661 | p1.(A)5 |
| 15 | 40915672 | 40915677 | p1.(A)6 |
| 15 | 40915709 | 40915713 | p1.(A)5 |
| 15 | 40916044 | 40916049 | p1.(A)6 |
| 15 | 40916100 | 40916104 | p1.(A)5 |
| 15 | 40916265 | 40916269 | p1.(A)5 |
| 15 | 40916445 | 40916449 | p1.(A)5 |
| 15 | 40916692 | 40916696 | p1.(G)5 |
| 15 | 40916822 | 40916827 | p1.(A)6 |
| 15 | 40916972 | 40916977 | p1.(A)6 |
| 15 | 40917160 | 40917164 | p1.(A)5 |
| 15 | 40917173 | 40917177 | p1.(T)5 |
| 15 | 40917332 | 40917336 | p1.(A)5 |
| 15 | 40917381 | 40917385 | p1.(T)5 |
| 15 | 40917419 | 40917423 | p1.(T)5 |
| 15 | 40917690 | 40917694 | p1.(A)5 |
| 15 | 40917724 | 40917728 | p1.(A)5 |
| 15 | 40917741 | 40917746 | p1.(A)6 |
| 15 | 40917759 | 40917770 | p3.(AAG)4 |
| 15 | 40917785 | 40917791 | p1.(A)7 |
| 15 | 40917796 | 40917801 | p1.(A)6 |
| 15 | 40920269 | 40920273 | p1.(T)5 |
| 15 | 40920324 | 40920329 | p1.(A)6 |
| 15 | 40933097 | 40933105 | p1.(T)9 |
| 15 | 40954465 | 40954469 | p1.(T)5 |
| 15 | 40990947 | 40990951 | p1.(T)5 |
| 15 | 40998365 | 40998369 | p1.(T)5 |
| 15 | 41001215 | 41001220 | p1.(T)6 |
| 15 | 41022126 | 41022131 | p1.(A)6 |
| 15 | 41023386 | 41023390 | p1.(T)5 |
| 15 | 41023399 | 41023403 | p1.(A)5 |
| 15 | 45847654 | 45847669 | p4.(TTTA)4 |
| 15 | 45847739 | 45847744 | p1.(A)6 |
| 15 | 45848143 | 45848147 | p1.(T)5 |
| 15 | 55700942 | 55700947 | p1.(A)6 |
| 15 | 55710625 | 55710629 | p1.(T)5 |
| 15 | 57355941 | 57355945 | p1.(T)5 |
| 15 | 57383979 | 57383983 | p1.(C)5 |
| 15 | 57489984 | 57489990 | p1.(A)7 |
| 15 | 57524477 | 57524481 | p1.(T)5 |
| 15 | 57525061 | 57525065 | p1.(T)5 |
| 15 | 57544619 | 57544623 | p1.(A)5 |
| 15 | 57565386 | 57565390 | p1.(A)5 |
| 15 | 57574630 | 57574634 | p1.(C)5 |
| 15 | 57574691 | 57574695 | p1.(A)5 |
| 15 | 57578878 | 57578882 | p1.(A)5 |
| 15 | 66679650 | 66679655 | p1.(C)6 |
| 15 | 66679726 | 66679730 | p1.(C)5 |
| 15 | 66727506 | 66727510 | p1.(G)5 |
| 15 | 66777509 | 66777513 | p1.(C)5 |
| 15 | 66779599 | 66779603 | p1.(T)5 |
| 15 | 66782062 | 66782067 | p1.(A)6 |
| 15 | 74287226 | 74287231 | p1.(C)6 |
| 15 | 74287236 | 74287240 | p1.(C)5 |
| 15 | 74290338 | 74290342 | p1.(C)5 |
| 15 | 74290349 | 74290353 | p1.(C)5 |
| 15 | 74290537 | 74290541 | p1.(T)5 |
| 15 | 74335425 | 74335429 | p1.(C)5 |
| 15 | 74336739 | 74336743 | p1.(G)5 |
| 15 | 74336986 | 74336990 | p1.(C)5 |
| 15 | 74337151 | 74337156 | p1.(G)6 |
| 15 | 74337203 | 74337207 | p1.(C)5 |
| 15 | 74337355 | 74337359 | p1.(G)5 |
| 15 | 88423489 | 88423498 | p1.(C)5(T)5 |
| 15 | 88476295 | 88476299 | p1.(G)5 |
| 15 | 88576130 | 88576134 | p1.(G)5 |
| 15 | 88678391 | 88678395 | p1.(T)5 |
| 15 | 88678547 | 88678552 | p1.(G)6 |
| 15 | 88678622 | 88678626 | p1.(G)5 |
| 15 | 88680722 | 88680726 | p1.(C)5 |
| 15 | 88680772 | 88680776 | p1.(A)5 |
| 15 | 88726662 | 88726666 | p1.(G)5 |
| 15 | 88799219 | 88799223 | p1.(G)5 |
| 15 | 90627494 | 90627498 | p1.(C)5 |
| 15 | 90628262 | 90628266 | p1.(C)5 |
| 15 | 90628339 | 90628343 | p1.(G)5 |
| 15 | 90630504 | 90630508 | p1.(A)5 |
| 15 | 90630813 | 90630817 | p1.(G)5 |
| 15 | 90631886 | 90631890 | p1.(T)5 |
| 15 | 90631918 | 90631924 | p1.(C)7 |
| 15 | 90634774 | 90634778 | p1.(G)5 |
| 15 | 90645536 | 90645540 | p1.(G)5 |
| 15 | 91136948 | 91136952 | p1.(C)5 |
| 15 | 91136994 | 91136999 | p1.(T)6 |
| 15 | 91147608 | 91147612 | p1.(T)5 |
| 15 | 91150677 | 91150681 | p1.(G)5 |
| 15 | 91161098 | 91161106 | p1.(T)9 |
| 15 | 91168999 | 91169004 | p1.(T)6 |
| 15 | 91181747 | 91181751 | p1.(C)5 |
| 15 | 91292589 | 91292593 | p1.(T)5 |
| 15 | 91292610 | 91292614 | p1.(A)5 |
| 15 | 91292663 | 91292667 | p1.(A)5 |
| 15 | 91292769 | 91292773 | p1.(A)5 |
| 15 | 91293056 | 91293061 | p1.(A)6 |
| 15 | 91293075 | 91293080 | p1.(T)6 |
| 15 | 91295090 | 91295101 | p3.(TGA)4 |
| 15 | 91303965 | 91303969 | p1.(T)5 |
| 15 | 91304060 | 91304064 | p1.(T)5 |
| 15 | 91304139 | 91304147 | p1.(A)9 |
| 15 | 91304286 | 91304300 | p3.(TGA)5 |
| 15 | 91306286 | 91306290 | p1.(T)5 |
| 15 | 91306294 | 91306299 | p1.(A)6 |
| 15 | 91306395 | 91306399 | p1.(T)5 |
| 15 | 91312651 | 91312659 | p1.(T)9 |
| 15 | 91326044 | 91326049 | p1.(T)6 |
| 15 | 91326101 | 91326105 | p1.(A)5 |
| 15 | 91326110 | 91326114 | p1.(A)5 |
| 15 | 91328318 | 91328322 | p1.(T)5 |
| 15 | 91341407 | 91341411 | p1.(T)5 |
| 15 | 91346764 | 91346768 | p1.(A)5 |
| 15 | 91346826 | 91346835 | p1.(T)5(A)5 |
| 15 | 91346959 | 91346963 | p1.(T)5 |
| 15 | 91347433 | 91347438 | p1.(A)6 |
| 15 | 91347440 | 91347444 | p1.(A)5 |
| 15 | 91347484 | 91347489 | p1.(A)6 |
| 15 | 91347533 | 91347537 | p1.(T)5 |
| 15 | 91347557 | 91347561 | p1.(T)5 |
| 15 | 91352430 | 91352434 | p1.(A)5 |
| 15 | 91354569 | 91354573 | p1.(A)5 |
| 15 | 91354604 | 91354608 | p1.(A)5 |
| 15 | 91358331 | 91358335 | p1.(G)5 |
| 15 | 99192755 | 99192778 | p1.(T)24 |
| 15 | 99192851 | 99192855 | p1.(G)5 |
| 15 | 99251109 | 99251114 | p1.(G)6 |
| 15 | 99251129 | 99251133 | p1.(A)5 |
| 15 | 99251216 | 99251220 | p1.(C)5 |
| 15 | 99434695 | 99434706 | p4.(TGCC)3 |
| 15 | 99440129 | 99440133 | p1.(G)5 |
| 15 | 99442809 | 99442814 | p1.(A)6 |
| 15 | 99454621 | 99454625 | p1.(C)5 |
| 15 | 99467164 | 99467168 | p1.(T)5 |
| 15 | 99473454 | 99473458 | p1.(T)5 |
| 15 | 99500476 | 99500480 | p1.(C)5 |
| 15 | 99500618 | 99500622 | p1.(G)5 |
| 16 | 339452 | 339456 | p1.(T)5 |
| 16 | 343564 | 343568 | p1.(G)5 |
| 16 | 347093 | 347097 | p1.(C)5 |
| 16 | 347748 | 347752 | p1.(G)5 |
| 16 | 347983 | 347988 | p1.(C)6 |
| 16 | 348180 | 348184 | p1.(G)5 |
| 16 | 348211 | 348215 | p1.(G)5 |
| 16 | 360055 | 360060 | p1.(G)6 |
| 16 | 396234 | 396239 | p1.(G)6 |
| 16 | 396792 | 396796 | p1.(G)5 |
| 16 | 396955 | 396959 | p1.(G)5 |
| 16 | 2100466 | 2100470 | p1.(A)5 |
| 16 | 2104308 | 2104312 | p1.(G)5 |
| 16 | 2105396 | 2105400 | p1.(C)5 |
| 16 | 2110822 | 2110826 | p1.(G)5 |
| 16 | 2112016 | 2112020 | p1.(C)5 |
| 16 | 2112605 | 2112609 | p1.(G)5 |
| 16 | 2115544 | 2115548 | p1.(C)5 |
| 16 | 2121821 | 2121825 | p1.(C)5 |
| 16 | 2126529 | 2126533 | p1.(C)5 |
| 16 | 2127615 | 2127620 | p1.(C)6 |
| 16 | 2129112 | 2129116 | p1.(A)5 |
| 16 | 2129400 | 2129404 | p1.(G)5 |
| 16 | 2130165 | 2130169 | p1.(G)5 |
| 16 | 2131772 | 2131776 | p1.(C)5 |
| 16 | 2133752 | 2133756 | p1.(C)5 |
| 16 | 2134569 | 2134574 | p1.(C)6 |
| 16 | 2134978 | 2134982 | p1.(C)5 |
| 16 | 2138147 | 2138151 | p1.(G)5 |
| 16 | 2213961 | 2213966 | p1.(G)6 |
| 16 | 2220615 | 2220620 | p1.(C)6 |
| 16 | 2220714 | 2220728 | p3.(GAG)5 |
| 16 | 2220740 | 2220744 | p1.(G)5 |
| 16 | 2222314 | 2222319 | p1.(C)6 |
| 16 | 2222334 | 2222338 | p1.(C)5 |
| 16 | 2226259 | 2226263 | p1.(C)5 |
| 16 | 3777751 | 3777755 | p1.(C)5 |
| 16 | 3777803 | 3777807 | p1.(G)5 |
| 16 | 3777817 | 3777821 | p1.(G)5 |
| 16 | 3777898 | 3777903 | p1.(G)6 |
| 16 | 3778099 | 3778103 | p1.(G)5 |
| 16 | 3778193 | 3778197 | p1.(G)5 |
| 16 | 3778207 | 3778211 | p1.(C)5 |
| 16 | 3778282 | 3778286 | p1.(G)5 |
| 16 | 3778303 | 3778314 | p3.(GCT)4 |
| 16 | 3778377 | 3778381 | p1.(C)5 |
| 16 | 3778401 | 3778412 | p3.(TGC)4 |
| 16 | 3778440 | 3778454 | p3.(TGC)5 |
| 16 | 3778549 | 3778553 | p1.(G)5 |
| 16 | 3779057 | 3779061 | p1.(G)5 |
| 16 | 3779100 | 3779104 | p1.(G)5 |
| 16 | 3779136 | 3779147 | p3.(TGC)4 |
| 16 | 3779193 | 3779197 | p1.(G)5 |
| 16 | 3779205 | 3779209 | p1.(G)5 |
| 16 | 3779211 | 3779217 | p1.(G)7 |
| 16 | 3779258 | 3779263 | p1.(G)6 |
| 16 | 3779331 | 3779335 | p1.(G)5 |
| 16 | 3779369 | 3779373 | p1.(G)5 |
| 16 | 3779604 | 3779608 | p1.(C)5 |
| 16 | 3779755 | 3779759 | p1.(G)5 |
| 16 | 3779810 | 3779814 | p1.(C)5 |
| 16 | 3781402 | 3781406 | p1.(G)5 |
| 16 | 3781421 | 3781426 | p1.(G)6 |
| 16 | 3786071 | 3786075 | p1.(T)5 |
| 16 | 3786697 | 3786701 | p1.(T)5 |
| 16 | 3786734 | 3786739 | p1.(T)6 |
| 16 | 3789591 | 3789596 | p1.(G)6 |
| 16 | 3789629 | 3789634 | p1.(A)6 |
| 16 | 3790459 | 3790463 | p1.(A)5 |
| 16 | 3790496 | 3790500 | p1.(A)5 |
| 16 | 3808053 | 3808065 | p1.(A)13 |
| 16 | 3817721 | 3817727 | p1.(T)7 |
| 16 | 3817917 | 3817921 | p1.(A)5 |
| 16 | 3819310 | 3819314 | p1.(G)5 |
| 16 | 3819360 | 3819365 | p1.(A)6 |
| 16 | 3820641 | 3820645 | p1.(G)5 |
| 16 | 3828047 | 3828051 | p1.(G)5 |
| 16 | 3828057 | 3828061 | p1.(C)5 |
| 16 | 3828121 | 3828125 | p1.(T)5 |
| 16 | 3828136 | 3828140 | p1.(T)5 |
| 16 | 3828186 | 3828190 | p1.(A)5 |
| 16 | 3832688 | 3832692 | p1.(G)5 |
| 16 | 3842008 | 3842012 | p1.(T)5 |
| 16 | 3843434 | 3843438 | p1.(T)5 |
| 16 | 3843553 | 3843557 | p1.(T)5 |
| 16 | 3860693 | 3860697 | p1.(G)5 |
| 16 | 3860788 | 3860793 | p1.(A)6 |
| 16 | 3900661 | 3900665 | p1.(G)5 |
| 16 | 3900703 | 3900707 | p1.(G)5 |
| 16 | 3929821 | 3929825 | p1.(C)5 |
| 16 | 9857003 | 9857007 | p1.(T)5 |
| 16 | 9857075 | 9857079 | p1.(G)5 |
| 16 | 9857365 | 9857369 | p1.(G)5 |
| 16 | 9857380 | 9857386 | p1.(T)7 |
| 16 | 9857428 | 9857432 | p1.(A)5 |
| 16 | 9857702 | 9857706 | p1.(G)5 |
| 16 | 9857917 | 9857921 | p1.(C)5 |
| 16 | 9858533 | 9858537 | p1.(A)5 |
| 16 | 9858693 | 9858697 | p1.(T)5 |
| 16 | 9923281 | 9923285 | p1.(T)5 |
| 16 | 9928017 | 9928021 | p1.(A)5 |
| 16 | 9934970 | 9934974 | p1.(A)5 |
| 16 | 9943668 | 9943672 | p1.(G)5 |
| 16 | 10273880 | 10273884 | p1.(C)5 |
| 16 | 10274173 | 10274178 | p1.(G)6 |
| 16 | 10989215 | 10989219 | p1.(C)5 |
| 16 | 10995900 | 10995905 | p1.(C)6 |
| 16 | 10996050 | 10996054 | p1.(G)5 |
| 16 | 10997576 | 10997580 | p1.(C)5 |
| 16 | 10997614 | 10997618 | p1.(C)5 |
| 16 | 10998613 | 10998617 | p1.(C)5 |
| 16 | 11000726 | 11000730 | p1.(G)5 |
| 16 | 11000926 | 11000930 | p1.(G)5 |
| 16 | 11001305 | 11001311 | p1.(C)7 |
| 16 | 11001500 | 11001504 | p1.(G)5 |
| 16 | 11009445 | 11009449 | p1.(C)5 |
| 16 | 11010287 | 11010291 | p1.(C)5 |
| 16 | 11348750 | 11348754 | p1.(G)5 |
| 16 | 11348846 | 11348850 | p1.(C)5 |
| 16 | 11348998 | 11349002 | p1.(A)5 |
| 16 | 11349090 | 11349094 | p1.(C)5 |
| 16 | 11349186 | 11349190 | p1.(G)5 |
| 16 | 11349228 | 11349232 | p1.(G)5 |
| 16 | 11349377 | 11349381 | p1.(G)5 |
| 16 | 11439367 | 11439371 | p1.(G)5 |
| 16 | 11444490 | 11444494 | p1.(T)5 |
| 16 | 11444668 | 11444672 | p1.(A)5 |
| 16 | 12059171 | 12059175 | p1.(T)5 |
| 16 | 12060144 | 12060148 | p1.(T)5 |
| 16 | 12060189 | 12060193 | p1.(A)5 |
| 16 | 12096835 | 12096839 | p1.(T)5 |
| 16 | 12142216 | 12142222 | p1.(T)7 |
| 16 | 12145684 | 12145698 | p1.(T)15 |
| 16 | 12145715 | 12145719 | p1.(A)5 |
| 16 | 12145733 | 12145737 | p1.(A)5 |
| 16 | 12145802 | 12145806 | p1.(A)5 |
| 16 | 14020409 | 14020413 | p1.(C)5 |
| 16 | 14020492 | 14020496 | p1.(A)5 |
| 16 | 14020551 | 14020555 | p1.(T)5 |
| 16 | 14020583 | 14020587 | p1.(T)5 |
| 16 | 14021907 | 14021911 | p1.(T)5 |
| 16 | 14022099 | 14022103 | p1.(T)5 |
| 16 | 14024724 | 14024728 | p1.(A)5 |
| 16 | 14026097 | 14026102 | p1.(A)6 |
| 16 | 14026104 | 14026109 | p1.(A)6 |
| 16 | 14026116 | 14026121 | p1.(A)6 |
| 16 | 14028053 | 14028058 | p1.(A)6 |
| 16 | 14028165 | 14028170 | p1.(A)6 |
| 16 | 14029202 | 14029206 | p1.(C)5 |
| 16 | 14029245 | 14029249 | p1.(A)5 |
| 16 | 14029251 | 14029256 | p1.(A)6 |
| 16 | 14031703 | 14031707 | p1.(A)5 |
| 16 | 14038582 | 14038586 | p1.(A)5 |
| 16 | 14041458 | 14041462 | p1.(T)5 |
| 16 | 14042012 | 14042016 | p1.(A)5 |
| 16 | 14042034 | 14042038 | p1.(A)5 |
| 16 | 14042196 | 14042200 | p1.(A)5 |
| 16 | 15797783 | 15797790 | p1.(T)8 |
| 16 | 15797788 | 15797799 | p4.(TTTG)3 |
| 16 | 15797806 | 15797810 | p1.(T)5 |
| 16 | 15811082 | 15811086 | p1.(C)5 |
| 16 | 15812298 | 15812302 | p1.(G)5 |
| 16 | 15815472 | 15815476 | p1.(T)5 |
| 16 | 15820795 | 15820806 | p3.(CTT)4 |
| 16 | 15826409 | 15826413 | p1.(A)5 |
| 16 | 15831482 | 15831486 | p1.(A)5 |
| 16 | 15832454 | 15832458 | p1.(T)5 |
| 16 | 15832547 | 15832551 | p1.(T)5 |
| 16 | 15834017 | 15834028 | p3.(TCC)4 |
| 16 | 15835375 | 15835386 | p3.(TCC)4 |
| 16 | 15839102 | 15839107 | p1.(G)6 |
| 16 | 15870020 | 15870024 | p1.(T)5 |
| 16 | 15931944 | 15931948 | p1.(C)5 |
| 16 | 15932054 | 15932058 | p1.(T)5 |
| 16 | 23614813 | 23614817 | p1.(T)5 |
| 16 | 23625383 | 23625387 | p1.(T)5 |
| 16 | 23625402 | 23625406 | p1.(T)5 |
| 16 | 23632770 | 23632774 | p1.(G)5 |
| 16 | 23632779 | 23632783 | p1.(A)5 |
| 16 | 23637581 | 23637585 | p1.(T)5 |
| 16 | 23641240 | 23641244 | p1.(T)5 |
| 16 | 23641510 | 23641514 | p1.(A)5 |
| 16 | 23641551 | 23641555 | p1.(T)5 |
| 16 | 23641576 | 23641580 | p1.(T)5 |
| 16 | 23641597 | 23641601 | p1.(T)5 |
| 16 | 23641768 | 23641772 | p1.(T)5 |
| 16 | 23646202 | 23646206 | p1.(T)5 |
| 16 | 23646321 | 23646325 | p1.(T)5 |
| 16 | 23646518 | 23646522 | p1.(T)5 |
| 16 | 23646536 | 23646540 | p1.(T)5 |
| 16 | 23646542 | 23646546 | p1.(T)5 |
| 16 | 23646553 | 23646558 | p1.(T)6 |
| 16 | 23646688 | 23646692 | p1.(T)5 |
| 16 | 23646981 | 23646987 | p1.(T)7 |
| 16 | 23647028 | 23647033 | p1.(T)6 |
| 16 | 23647174 | 23647178 | p1.(T)5 |
| 16 | 23647310 | 23647314 | p1.(T)5 |
| 16 | 23647646 | 23647650 | p1.(T)5 |
| 16 | 23652421 | 23652425 | p1.(C)5 |
| 16 | 27441412 | 27441416 | p1.(C)5 |
| 16 | 27454270 | 27454274 | p1.(T)5 |
| 16 | 27454294 | 27454298 | p1.(C)5 |
| 16 | 27455913 | 27455917 | p1.(C)5 |
| 16 | 27457319 | 27457323 | p1.(C)5 |
| 16 | 27460061 | 27460066 | p1.(G)6 |
| 16 | 27460368 | 27460372 | p1.(G)5 |
| 16 | 27460530 | 27460536 | p1.(C)7 |
| 16 | 31191504 | 31191515 | p4.(TTGC)3 |
| 16 | 31195645 | 31195649 | p1.(C)5 |
| 16 | 31201632 | 31201643 | p3.(GTG)4 |
| 16 | 31202139 | 31202143 | p1.(G)5 |
| 16 | 31202283 | 31202287 | p1.(G)5 |
| 16 | 31202376 | 31202380 | p1.(G)5 |
| 16 | 50783635 | 50783639 | p1.(A)5 |
| 16 | 50783668 | 50783673 | p1.(T)6 |
| 16 | 50783808 | 50783812 | p1.(A)5 |
| 16 | 50783825 | 50783829 | p1.(A)5 |
| 16 | 50783949 | 50783953 | p1.(A)5 |
| 16 | 50784028 | 50784032 | p1.(A)5 |
| 16 | 50785506 | 50785510 | p1.(A)5 |
| 16 | 50785567 | 50785571 | p1.(T)5 |
| 16 | 50813568 | 50813573 | p1.(T)6 |
| 16 | 50815149 | 50815153 | p1.(T)5 |
| 16 | 50816282 | 50816286 | p1.(A)5 |
| 16 | 50816290 | 50816294 | p1.(A)5 |
| 16 | 50816386 | 50816390 | p1.(A)5 |
| 16 | 50820787 | 50820791 | p1.(A)5 |
| 16 | 50820852 | 50820856 | p1.(A)5 |
| 16 | 50825462 | 50825466 | p1.(T)5 |
| 16 | 50825510 | 50825514 | p1.(T)5 |
| 16 | 50825519 | 50825525 | p1.(A)7 |
| 16 | 50827539 | 50827543 | p1.(A)5 |
| 16 | 56832375 | 56832379 | p1.(C)5 |
| 16 | 56852600 | 56852604 | p1.(C)5 |
| 16 | 56862880 | 56862884 | p1.(T)5 |
| 16 | 56871570 | 56871574 | p1.(C)5 |
| 16 | 56872937 | 56872942 | p1.(T)6 |
| 16 | 56966143 | 56966154 | p3.(CCG)4 |
| 16 | 56969317 | 56969321 | p1.(A)5 |
| 16 | 56969363 | 56969367 | p1.(A)5 |
| 16 | 56973795 | 56973799 | p1.(T)5 |
| 16 | 56974079 | 56974083 | p1.(T)5 |
| 16 | 56974134 | 56974138 | p1.(G)5 |
| 16 | 56977181 | 56977186 | p1.(C)6 |
| 16 | 64981820 | 64981824 | p1.(G)5 |
| 16 | 64981894 | 64981898 | p1.(C)5 |
| 16 | 65005526 | 65005530 | p1.(G)5 |
| 16 | 65006905 | 65006909 | p1.(A)5 |
| 16 | 65016062 | 65016066 | p1.(G)5 |
| 16 | 65022061 | 65022065 | p1.(T)5 |
| 16 | 65022077 | 65022081 | p1.(C)5 |
| 16 | 65022250 | 65022261 | p3.(AGA)4 |
| 16 | 65026836 | 65026840 | p1.(A)5 |
| 16 | 65032685 | 65032689 | p1.(A)5 |
| 16 | 65038686 | 65038690 | p1.(C)5 |
| 16 | 65038772 | 65038776 | p1.(T)5 |
| 16 | 65087836 | 65087840 | p1.(A)5 |
| 16 | 65155906 | 65155910 | p1.(C)5 |
| 16 | 67063280 | 67063291 | p4.(CGGC)3 |
| 16 | 67063359 | 67063364 | p1.(T)6 |
| 16 | 67063397 | 67063408 | p4.(GGCG)3 |
| 16 | 67070530 | 67070534 | p1.(T)5 |
| 16 | 67070578 | 67070583 | p1.(T)6 |
| 16 | 67644826 | 67644830 | p1.(G)5 |
| 16 | 67644874 | 67644878 | p1.(G)5 |
| 16 | 67645140 | 67645144 | p1.(G)5 |
| 16 | 67645242 | 67645246 | p1.(G)5 |
| 16 | 67645310 | 67645314 | p1.(A)5 |
| 16 | 67645339 | 67645345 | p1.(A)7 |
| 16 | 67645357 | 67645361 | p1.(A)5 |
| 16 | 67645500 | 67645504 | p1.(A)5 |
| 16 | 67645507 | 67645511 | p1.(A)5 |
| 16 | 67646013 | 67646022 | p2.(AC)5 |
| 16 | 67655481 | 67655485 | p1.(A)5 |
| 16 | 67663291 | 67663295 | p1.(T)5 |
| 16 | 67663351 | 67663355 | p1.(G)5 |
| 16 | 67663393 | 67663397 | p1.(A)5 |
| 16 | 67670718 | 67670722 | p1.(C)5 |
| 16 | 67671655 | 67671661 | p1.(A)7 |
| 16 | 67671723 | 67671727 | p1.(C)5 |
| 16 | 68772188 | 68772192 | p1.(C)5 |
| 16 | 68835613 | 68835617 | p1.(T)5 |
| 16 | 68835781 | 68835786 | p1.(C)6 |
| 16 | 68842439 | 68842443 | p1.(A)5 |
| 16 | 68842456 | 68842460 | p1.(A)5 |
| 16 | 68842662 | 68842666 | p1.(C)5 |
| 16 | 68845694 | 68845698 | p1.(A)5 |
| 16 | 68847286 | 68847290 | p1.(C)5 |
| 16 | 68849545 | 68849549 | p1.(C)5 |
| 16 | 68853333 | 68853337 | p1.(G)5 |
| 16 | 68855967 | 68855971 | p1.(C)5 |
| 16 | 68862139 | 68862143 | p1.(C)5 |
| 16 | 68863543 | 68863548 | p1.(T)6 |
| 16 | 68863643 | 68863647 | p1.(C)5 |
| 16 | 79632685 | 79632689 | p1.(A)5 |
| 16 | 79632907 | 79632911 | p1.(T)5 |
| 16 | 79633069 | 79633074 | p1.(C)6 |
| 16 | 79633076 | 79633080 | p1.(C)5 |
| 16 | 79633587 | 79633591 | p1.(G)5 |
| 16 | 79633594 | 79633598 | p1.(G)5 |
| 16 | 79633623 | 79633627 | p1.(G)5 |
| 16 | 79633642 | 79633646 | p1.(C)5 |
| 16 | 79633699 | 79633703 | p1.(T)5 |
| 16 | 79633806 | 79633829 | p3.(GCC)8 |
| 16 | 88943420 | 88943424 | p1.(G)5 |
| 16 | 88943690 | 88943694 | p1.(G)5 |
| 16 | 88947117 | 88947121 | p1.(C)5 |
| 16 | 88947900 | 88947904 | p1.(G)5 |
| 16 | 88949178 | 88949182 | p1.(G)5 |
| 16 | 88951484 | 88951488 | p1.(G)5 |
| 16 | 88952431 | 88952435 | p1.(G)5 |
| 16 | 88958773 | 88958777 | p1.(G)5 |
| 16 | 88967899 | 88967903 | p1.(G)5 |
| 16 | 88967931 | 88967937 | p1.(G)7 |
| 16 | 88967945 | 88967949 | p1.(C)5 |
| 16 | 88967968 | 88967972 | p1.(G)5 |
| 16 | 88967982 | 88967987 | p1.(G)6 |
| 16 | 89805312 | 89805316 | p1.(T)5 |
| 16 | 89805344 | 89805348 | p1.(A)5 |
| 16 | 89805890 | 89805894 | p1.(A)5 |
| 16 | 89816314 | 89816318 | p1.(A)5 |
| 16 | 89825051 | 89825055 | p1.(C)5 |
| 16 | 89831345 | 89831354 | p2.(AG)5 |
| 16 | 89836978 | 89836982 | p1.(G)5 |
| 16 | 89858961 | 89858965 | p1.(G)5 |
| 16 | 89869675 | 89869680 | p1.(T)6 |
| 16 | 89869710 | 89869714 | p1.(T)5 |
| 16 | 89869753 | 89869757 | p1.(A)5 |
| 16 | 89874779 | 89874783 | p1.(A)5 |
| 16 | 89874780 | 89874794 | p5.(AAAAC)3 |
| 16 | 89877444 | 89877448 | p1.(C)5 |
| 16 | 89880998 | 89881003 | p1.(T)6 |
| 16 | 89882372 | 89882376 | p1.(T)5 |
| 16 | 89882974 | 89882978 | p1.(C)5 |
| 17 | 1264432 | 1264436 | p1.(A)5 |
| 17 | 1268176 | 1268180 | p1.(T)5 |
| 17 | 1303327 | 1303333 | p1.(C)7 |
| 17 | 5039922 | 5039926 | p1.(G)5 |
| 17 | 5041562 | 5041566 | p1.(C)5 |
| 17 | 5042701 | 5042705 | p1.(T)5 |
| 17 | 5042861 | 5042865 | p1.(G)5 |
| 17 | 5042875 | 5042879 | p1.(C)5 |
| 17 | 5050397 | 5050401 | p1.(A)5 |
| 17 | 5050423 | 5050427 | p1.(T)5 |
| 17 | 5072157 | 5072162 | p1.(T)6 |
| 17 | 5072211 | 5072215 | p1.(G)5 |
| 17 | 5074903 | 5074907 | p1.(G)5 |
| 17 | 5074927 | 5074931 | p1.(A)5 |
| 17 | 5211975 | 5211986 | p1.(T)7(C)5 |
| 17 | 5212019 | 5212024 | p1.(A)6 |
| 17 | 5212036 | 5212040 | p1.(T)5 |
| 17 | 5238634 | 5238638 | p1.(A)5 |
| 17 | 5241301 | 5241305 | p1.(T)5 |
| 17 | 5266262 | 5266266 | p1.(A)5 |
| 17 | 5271654 | 5271660 | p1.(T)7 |
| 17 | 5283649 | 5283653 | p1.(T)5 |
| 17 | 5284676 | 5284681 | p1.(T)6 |
| 17 | 5286554 | 5286558 | p1.(T)5 |
| 17 | 7572885 | 7572889 | p1.(G)5 |
| 17 | 7572963 | 7572968 | p1.(T)6 |
| 17 | 7572991 | 7572995 | p1.(T)5 |
| 17 | 7573944 | 7573949 | p1.(C)6 |
| 17 | 7577036 | 7577040 | p1.(G)5 |
| 17 | 7578398 | 7578402 | p1.(G)5 |
| 17 | 7578475 | 7578479 | p1.(G)5 |
| 17 | 7579420 | 7579424 | p1.(G)5 |
| 17 | 7579471 | 7579476 | p1.(G)6 |
| 17 | 7579585 | 7579589 | p1.(G)5 |
| 17 | 7579875 | 7579879 | p1.(G)5 |
| 17 | 8044444 | 8044455 | p3.(TCC)4 |
| 17 | 8045990 | 8045994 | p1.(C)5 |
| 17 | 8046624 | 8046628 | p1.(G)5 |
| 17 | 8046664 | 8046668 | p1.(C)5 |
| 17 | 8046676 | 8046680 | p1.(G)5 |
| 17 | 8046936 | 8046940 | p1.(G)5 |
| 17 | 8046957 | 8046961 | p1.(G)5 |
| 17 | 8046970 | 8046974 | p1.(G)5 |
| 17 | 8047028 | 8047032 | p1.(G)5 |
| 17 | 8047055 | 8047059 | p1.(G)5 |
| 17 | 8047170 | 8047174 | p1.(G)5 |
| 17 | 8049284 | 8049288 | p1.(G)5 |
| 17 | 8049423 | 8049427 | p1.(C)5 |
| 17 | 8049759 | 8049773 | p3.(AGG)5 |
| 17 | 8050258 | 8050262 | p1.(A)5 |
| 17 | 8050599 | 8050603 | p1.(C)5 |
| 17 | 8050857 | 8050861 | p1.(G)5 |
| 17 | 8050864 | 8050868 | p1.(G)5 |
| 17 | 8050889 | 8050894 | p1.(G)6 |
| 17 | 8051378 | 8051382 | p1.(C)5 |
| 17 | 8051411 | 8051416 | p1.(G)6 |
| 17 | 8051562 | 8051566 | p1.(G)5 |
| 17 | 8053933 | 8053937 | p1.(G)5 |
| 17 | 8053954 | 8053958 | p1.(C)5 |
| 17 | 8053993 | 8053997 | p1.(C)5 |
| 17 | 8054012 | 8054016 | p1.(G)5 |
| 17 | 8108670 | 8108674 | p1.(G)5 |
| 17 | 8110130 | 8110135 | p1.(G)6 |
| 17 | 8110172 | 8110176 | p1.(A)5 |
| 17 | 8110209 | 8110213 | p1.(G)5 |
| 17 | 8113556 | 8113560 | p1.(G)5 |
| 17 | 9820503 | 9820508 | p1.(C)6 |
| 17 | 9820521 | 9820526 | p1.(C)6 |
| 17 | 9823027 | 9823031 | p1.(A)5 |
| 17 | 9846535 | 9846539 | p1.(G)5 |
| 17 | 9862544 | 9862548 | p1.(T)5 |
| 17 | 9923195 | 9923200 | p1.(G)6 |
| 17 | 11998914 | 11998918 | p1.(A)5 |
| 17 | 11998923 | 11998927 | p1.(A)5 |
| 17 | 12044602 | 12044607 | p1.(A)6 |
| 17 | 17117152 | 17117156 | p1.(A)5 |
| 17 | 17118487 | 17118491 | p1.(G)5 |
| 17 | 17119682 | 17119686 | p1.(G)5 |
| 17 | 17119709 | 17119716 | p1.(G)8 |
| 17 | 17129540 | 17129544 | p1.(G)5 |
| 17 | 17131219 | 17131223 | p1.(T)5 |
| 17 | 17131253 | 17131257 | p1.(C)5 |
| 17 | 17131403 | 17131407 | p1.(G)5 |
| 17 | 17942768 | 17942777 | p2.(TG)5 |
| 17 | 17942804 | 17942808 | p1.(G)5 |
| 17 | 17942910 | 17942914 | p1.(C)5 |
| 17 | 17942971 | 17942982 | p3.(GCG)4 |
| 17 | 17942997 | 17943002 | p1.(C)6 |
| 17 | 17943015 | 17943019 | p1.(G)5 |
| 17 | 17943049 | 17943053 | p1.(C)5 |
| 17 | 17943223 | 17943237 | p5.(GCCGG)3 |
| 17 | 17957429 | 17957433 | p1.(T)5 |
| 17 | 17957486 | 17957491 | p1.(A)6 |
| 17 | 17962196 | 17962200 | p1.(T)5 |
| 17 | 17968483 | 17968487 | p1.(C)5 |
| 17 | 17968598 | 17968603 | p1.(A)6 |
| 17 | 20000007 | 20000011 | p1.(G)5 |
| 17 | 20013796 | 20013800 | p1.(G)5 |
| 17 | 20108263 | 20108270 | p1.(A)8 |
| 17 | 20108745 | 20108749 | p1.(A)5 |
| 17 | 20108912 | 20108916 | p1.(A)5 |
| 17 | 20109003 | 20109007 | p1.(A)5 |
| 17 | 20130808 | 20130812 | p1.(A)5 |
| 17 | 20135629 | 20135640 | p3.(GGA)4 |
| 17 | 20135668 | 20135672 | p1.(G)5 |
| 17 | 20149324 | 20149328 | p1.(C)5 |
| 17 | 20150518 | 20150523 | p1.(T)6 |
| 17 | 20150565 | 20150569 | p1.(C)5 |
| 17 | 20156809 | 20156813 | p1.(T)5 |
| 17 | 20160801 | 20160805 | p1.(C)5 |
| 17 | 20160844 | 20160848 | p1.(C)5 |
| 17 | 29422307 | 29422312 | p1.(C)6 |
| 17 | 29482989 | 29482997 | p1.(T)9 |
| 17 | 29483009 | 29483013 | p1.(A)5 |
| 17 | 29486050 | 29486056 | p1.(A)7 |
| 17 | 29486095 | 29486099 | p1.(A)5 |
| 17 | 29496902 | 29496906 | p1.(T)5 |
| 17 | 29509514 | 29509518 | p1.(T)5 |
| 17 | 29528046 | 29528050 | p1.(T)5 |
| 17 | 29528062 | 29528066 | p1.(T)5 |
| 17 | 29533247 | 29533251 | p1.(T)5 |
| 17 | 29546008 | 29546014 | p1.(T)7 |
| 17 | 29552102 | 29552108 | p1.(T)7 |
| 17 | 29552144 | 29552149 | p1.(T)6 |
| 17 | 29553478 | 29553484 | p1.(C)7 |
| 17 | 29556175 | 29556179 | p1.(G)5 |
| 17 | 29556463 | 29556468 | p1.(T)6 |
| 17 | 29557946 | 29557952 | p1.(A)7 |
| 17 | 29560075 | 29560079 | p1.(A)5 |
| 17 | 29562958 | 29562962 | p1.(A)5 |
| 17 | 29575991 | 29575997 | p1.(T)7 |
| 17 | 29576098 | 29576103 | p1.(C)6 |
| 17 | 29585355 | 29585359 | p1.(T)5 |
| 17 | 29585473 | 29585477 | p1.(A)5 |
| 17 | 29587378 | 29587382 | p1.(T)5 |
| 17 | 29587388 | 29587392 | p1.(T)5 |
| 17 | 29592235 | 29592239 | p1.(T)5 |
| 17 | 29592261 | 29592265 | p1.(A)5 |
| 17 | 29592336 | 29592341 | p1.(T)6 |
| 17 | 29652996 | 29653000 | p1.(T)5 |
| 17 | 29654765 | 29654769 | p1.(C)5 |
| 17 | 29654863 | 29654868 | p1.(T)6 |
| 17 | 29657362 | 29657366 | p1.(A)5 |
| 17 | 29657480 | 29657484 | p1.(T)5 |
| 17 | 29661845 | 29661851 | p1.(T)7 |
| 17 | 29661917 | 29661921 | p1.(T)5 |
| 17 | 29661997 | 29662001 | p1.(A)5 |
| 17 | 29662025 | 29662029 | p1.(A)5 |
| 17 | 29664535 | 29664544 | p2.(GA)5 |
| 17 | 29664829 | 29664834 | p1.(T)6 |
| 17 | 29670156 | 29670161 | p1.(A)6 |
| 17 | 29676128 | 29676133 | p1.(T)6 |
| 17 | 29676272 | 29676276 | p1.(A)5 |
| 17 | 29683558 | 29683563 | p1.(C)6 |
| 17 | 29685482 | 29685490 | p1.(T)9 |
| 17 | 29701020 | 29701024 | p1.(T)5 |
| 17 | 29701175 | 29701186 | p4.(GCTT)3 |
| 17 | 29701189 | 29701194 | p1.(T)6 |
| 17 | 30264243 | 30264248 | p1.(G)6 |
| 17 | 30264290 | 30264294 | p1.(G)5 |
| 17 | 30264296 | 30264300 | p1.(G)5 |
| 17 | 30264326 | 30264330 | p1.(G)5 |
| 17 | 30264341 | 30264345 | p1.(G)5 |
| 17 | 30264407 | 30264411 | p1.(G)5 |
| 17 | 30264426 | 30264440 | p3.(CCT)5 |
| 17 | 30264457 | 30264461 | p1.(G)5 |
| 17 | 30315331 | 30315336 | p1.(T)6 |
| 17 | 30320877 | 30320881 | p1.(T)5 |
| 17 | 30321036 | 30321040 | p1.(T)5 |
| 17 | 30321571 | 30321575 | p1.(T)5 |
| 17 | 30321747 | 30321751 | p1.(A)5 |
| 17 | 30322768 | 30322773 | p1.(A)6 |
| 17 | 30325859 | 30325863 | p1.(A)5 |
| 17 | 30325872 | 30325876 | p1.(G)5 |
| 17 | 30326005 | 30326010 | p1.(A)6 |
| 17 | 30326012 | 30326016 | p1.(A)5 |
| 17 | 34144743 | 34144747 | p1.(G)5 |
| 17 | 34151072 | 34151077 | p1.(T)6 |
| 17 | 34151159 | 34151163 | p1.(G)5 |
| 17 | 34161549 | 34161563 | p5.(TTTTC)3 |
| 17 | 34163122 | 34163128 | p1.(T)7 |
| 17 | 34163252 | 34163256 | p1.(A)5 |
| 17 | 34165417 | 34165421 | p1.(T)5 |
| 17 | 34165493 | 34165497 | p1.(G)5 |
| 17 | 34169360 | 34169365 | p1.(T)6 |
| 17 | 34171073 | 34171077 | p1.(C)5 |
| 17 | 34171886 | 34171892 | p1.(G)7 |
| 17 | 34171993 | 34171997 | p1.(G)5 |
| 17 | 36864134 | 36864139 | p1.(C)6 |
| 17 | 36868121 | 36868125 | p1.(G)5 |
| 17 | 36868162 | 36868167 | p1.(G)6 |
| 17 | 36868998 | 36869003 | p1.(C)6 |
| 17 | 36871946 | 36871950 | p1.(A)5 |
| 17 | 36872060 | 36872065 | p1.(G)6 |
| 17 | 36872704 | 36872708 | p1.(C)5 |
| 17 | 36872745 | 36872750 | p1.(C)6 |
| 17 | 36872761 | 36872765 | p1.(T)5 |
| 17 | 36873054 | 36873058 | p1.(G)5 |
| 17 | 36873217 | 36873221 | p1.(C)5 |
| 17 | 36873777 | 36873781 | p1.(C)5 |
| 17 | 36874153 | 36874157 | p1.(C)5 |
| 17 | 36875803 | 36875807 | p1.(G)5 |
| 17 | 36876005 | 36876009 | p1.(C)5 |
| 17 | 36876058 | 36876062 | p1.(G)5 |
| 17 | 36880862 | 36880866 | p1.(C)5 |
| 17 | 36880948 | 36880952 | p1.(G)5 |
| 17 | 37070623 | 37070627 | p1.(G)5 |
| 17 | 37070690 | 37070701 | p3.(AGC)4 |
| 17 | 37074849 | 37074853 | p1.(C)5 |
| 17 | 37075042 | 37075046 | p1.(C)5 |
| 17 | 37618346 | 37618350 | p1.(G)5 |
| 17 | 37618494 | 37618498 | p1.(C)5 |
| 17 | 37618710 | 37618714 | p1.(A)5 |
| 17 | 37618716 | 37618721 | p1.(A)6 |
| 17 | 37618930 | 37618934 | p1.(A)5 |
| 17 | 37618990 | 37618994 | p1.(C)5 |
| 17 | 37619153 | 37619157 | p1.(C)5 |
| 17 | 37627123 | 37627127 | p1.(T)5 |
| 17 | 37627196 | 37627200 | p1.(A)5 |
| 17 | 37627289 | 37627293 | p1.(A)5 |
| 17 | 37627307 | 37627318 | p3.(GCT)4 |
| 17 | 37627361 | 37627365 | p1.(T)5 |
| 17 | 37627418 | 37627422 | p1.(A)5 |
| 17 | 37627446 | 37627450 | p1.(A)5 |
| 17 | 37627501 | 37627506 | p1.(A)6 |
| 17 | 37627688 | 37627692 | p1.(C)5 |
| 17 | 37627694 | 37627698 | p1.(C)5 |
| 17 | 37627730 | 37627734 | p1.(C)5 |
| 17 | 37627838 | 37627842 | p1.(C)5 |
| 17 | 37627892 | 37627896 | p1.(C)5 |
| 17 | 37646922 | 37646926 | p1.(C)5 |
| 17 | 37646973 | 37646977 | p1.(A)5 |
| 17 | 37646992 | 37646996 | p1.(T)5 |
| 17 | 37648997 | 37649001 | p1.(T)5 |
| 17 | 37649051 | 37649055 | p1.(G)5 |
| 17 | 37657637 | 37657641 | p1.(A)5 |
| 17 | 37672019 | 37672023 | p1.(T)5 |
| 17 | 37680918 | 37680922 | p1.(T)5 |
| 17 | 37680930 | 37680934 | p1.(C)5 |
| 17 | 37686892 | 37686896 | p1.(C)5 |
| 17 | 37686901 | 37686906 | p1.(C)6 |
| 17 | 37686931 | 37686935 | p1.(C)5 |
| 17 | 37686962 | 37686967 | p1.(C)6 |
| 17 | 37687472 | 37687478 | p1.(G)7 |
| 17 | 37687514 | 37687518 | p1.(G)5 |
| 17 | 37687540 | 37687544 | p1.(G)5 |
| 17 | 37856517 | 37856521 | p1.(G)5 |
| 17 | 37856540 | 37856545 | p1.(C)6 |
| 17 | 37864563 | 37864567 | p1.(C)5 |
| 17 | 37865605 | 37865609 | p1.(C)5 |
| 17 | 37866585 | 37866590 | p1.(C)6 |
| 17 | 37868224 | 37868228 | p1.(C)5 |
| 17 | 37873563 | 37873570 | p1.(C)8 |
| 17 | 37879565 | 37879569 | p1.(C)5 |
| 17 | 37879626 | 37879630 | p1.(G)5 |
| 17 | 37880235 | 37880239 | p1.(C)5 |
| 17 | 37881449 | 37881453 | p1.(G)5 |
| 17 | 37882045 | 37882049 | p1.(G)5 |
| 17 | 37882064 | 37882069 | p1.(C)6 |
| 17 | 37882888 | 37882892 | p1.(C)5 |
| 17 | 37883139 | 37883143 | p1.(G)5 |
| 17 | 37883215 | 37883219 | p1.(G)5 |
| 17 | 37883597 | 37883601 | p1.(C)5 |
| 17 | 37883664 | 37883668 | p1.(G)5 |
| 17 | 37883774 | 37883779 | p1.(C)6 |
| 17 | 37883790 | 37883794 | p1.(C)5 |
| 17 | 37883974 | 37883978 | p1.(C)5 |
| 17 | 37884080 | 37884084 | p1.(T)5 |
| 17 | 37884091 | 37884095 | p1.(G)5 |
| 17 | 37884218 | 37884223 | p1.(G)6 |
| 17 | 38487425 | 38487429 | p1.(C)5 |
| 17 | 38487504 | 38487508 | p1.(G)5 |
| 17 | 38487554 | 38487559 | p1.(C)6 |
| 17 | 38504624 | 38504628 | p1.(C)5 |
| 17 | 38508182 | 38508193 | p3.(AAG)4 |
| 17 | 38508244 | 38508248 | p1.(G)5 |
| 17 | 38508576 | 38508580 | p1.(C)5 |
| 17 | 38510655 | 38510659 | p1.(C)5 |
| 17 | 38510766 | 38510770 | p1.(G)5 |
| 17 | 38511612 | 38511616 | p1.(C)5 |
| 17 | 38512370 | 38512375 | p1.(G)6 |
| 17 | 38512377 | 38512382 | p1.(G)6 |
| 17 | 38512389 | 38512393 | p1.(G)5 |
| 17 | 38512402 | 38512408 | p1.(C)7 |
| 17 | 38512567 | 38512571 | p1.(C)5 |
| 17 | 38512607 | 38512611 | p1.(G)5 |
| 17 | 38512754 | 38512758 | p1.(G)5 |
| 17 | 38512832 | 38512836 | p1.(C)5 |
| 17 | 38512886 | 38512891 | p1.(C)6 |
| 17 | 38512911 | 38512915 | p1.(G)5 |
| 17 | 38512928 | 38512933 | p1.(G)6 |
| 17 | 38512936 | 38512940 | p1.(C)5 |
| 17 | 38512991 | 38512995 | p1.(T)5 |
| 17 | 38785043 | 38785048 | p1.(T)6 |
| 17 | 38785153 | 38785157 | p1.(C)5 |
| 17 | 38787185 | 38787191 | p1.(A)7 |
| 17 | 38792169 | 38792175 | p1.(T)7 |
| 17 | 38792285 | 38792289 | p1.(T)5 |
| 17 | 38792313 | 38792317 | p1.(G)5 |
| 17 | 38792680 | 38792684 | p1.(T)5 |
| 17 | 38792782 | 38792786 | p1.(A)5 |
| 17 | 38793793 | 38793797 | p1.(G)5 |
| 17 | 40354405 | 40354409 | p1.(G)5 |
| 17 | 40354436 | 40354440 | p1.(C)5 |
| 17 | 40362447 | 40362453 | p1.(T)7 |
| 17 | 40368127 | 40368131 | p1.(A)5 |
| 17 | 40369187 | 40369191 | p1.(A)5 |
| 17 | 40369241 | 40369245 | p1.(T)5 |
| 17 | 40370236 | 40370243 | p1.(G)8 |
| 17 | 40375459 | 40375464 | p1.(T)6 |
| 17 | 40379587 | 40379591 | p1.(A)5 |
| 17 | 40379600 | 40379604 | p1.(C)5 |
| 17 | 40379711 | 40379717 | p1.(A)7 |
| 17 | 40467769 | 40467773 | p1.(G)5 |
| 17 | 40468879 | 40468884 | p1.(G)6 |
| 17 | 40475305 | 40475309 | p1.(T)5 |
| 17 | 40476839 | 40476843 | p1.(G)5 |
| 17 | 40476850 | 40476855 | p1.(A)6 |
| 17 | 40476969 | 40476973 | p1.(G)5 |
| 17 | 40481444 | 40481448 | p1.(C)5 |
| 17 | 40485995 | 40485999 | p1.(T)5 |
| 17 | 40491420 | 40491424 | p1.(C)5 |
| 17 | 40491433 | 40491437 | p1.(A)5 |
| 17 | 41197709 | 41197713 | p1.(G)5 |
| 17 | 41215361 | 41215365 | p1.(T)5 |
| 17 | 41228526 | 41228530 | p1.(T)5 |
| 17 | 41234422 | 41234426 | p1.(T)5 |
| 17 | 41234597 | 41234601 | p1.(A)5 |
| 17 | 41242954 | 41242958 | p1.(T)5 |
| 17 | 41244017 | 41244021 | p1.(A)5 |
| 17 | 41244219 | 41244224 | p1.(T)6 |
| 17 | 41244440 | 41244444 | p1.(A)5 |
| 17 | 41244558 | 41244562 | p1.(T)5 |
| 17 | 41244596 | 41244600 | p1.(A)5 |
| 17 | 41245162 | 41245166 | p1.(T)5 |
| 17 | 41245355 | 41245359 | p1.(T)5 |
| 17 | 41245587 | 41245594 | p1.(T)8 |
| 17 | 41245725 | 41245729 | p1.(T)5 |
| 17 | 41245820 | 41245824 | p1.(T)5 |
| 17 | 41245848 | 41245852 | p1.(T)5 |
| 17 | 41246532 | 41246538 | p1.(T)7 |
| 17 | 41247865 | 41247870 | p1.(T)6 |
| 17 | 41256251 | 41256256 | p1.(T)6 |
| 17 | 41605867 | 41605871 | p1.(G)5 |
| 17 | 41605882 | 41605886 | p1.(G)5 |
| 17 | 41606496 | 41606500 | p1.(C)5 |
| 17 | 41607554 | 41607558 | p1.(G)5 |
| 17 | 41610084 | 41610088 | p1.(C)5 |
| 17 | 41610229 | 41610233 | p1.(G)5 |
| 17 | 41610251 | 41610255 | p1.(C)5 |
| 17 | 41610701 | 41610707 | p1.(G)7 |
| 17 | 41611358 | 41611362 | p1.(G)5 |
| 17 | 41623025 | 41623029 | p1.(G)5 |
| 17 | 41623042 | 41623046 | p1.(G)5 |
| 17 | 47677765 | 47677769 | p1.(G)5 |
| 17 | 47684696 | 47684700 | p1.(A)5 |
| 17 | 47700209 | 47700213 | p1.(G)5 |
| 17 | 48262813 | 48262818 | p1.(G)6 |
| 17 | 48262926 | 48262930 | p1.(G)5 |
| 17 | 48264249 | 48264253 | p1.(G)5 |
| 17 | 48264275 | 48264280 | p1.(G)6 |
| 17 | 48264413 | 48264417 | p1.(G)5 |
| 17 | 48264476 | 48264480 | p1.(G)5 |
| 17 | 48264491 | 48264495 | p1.(G)5 |
| 17 | 48265247 | 48265251 | p1.(G)5 |
| 17 | 48265329 | 48265333 | p1.(G)5 |
| 17 | 48265964 | 48265968 | p1.(G)5 |
| 17 | 48266153 | 48266157 | p1.(C)5 |
| 17 | 48266161 | 48266176 | p2.(AG)8 |
| 17 | 48266283 | 48266287 | p1.(G)5 |
| 17 | 48266301 | 48266306 | p1.(G)6 |
| 17 | 48266319 | 48266323 | p1.(G)5 |
| 17 | 48266516 | 48266521 | p1.(G)6 |
| 17 | 48266784 | 48266788 | p1.(G)5 |
| 17 | 48266793 | 48266797 | p1.(G)5 |
| 17 | 48266883 | 48266887 | p1.(G)5 |
| 17 | 48267221 | 48267225 | p1.(G)5 |
| 17 | 48267372 | 48267376 | p1.(G)5 |
| 17 | 48267399 | 48267403 | p1.(G)5 |
| 17 | 48267472 | 48267476 | p1.(G)5 |
| 17 | 48267904 | 48267908 | p1.(G)5 |
| 17 | 48269204 | 48269208 | p1.(G)5 |
| 17 | 48269390 | 48269394 | p1.(G)5 |
| 17 | 48270010 | 48270014 | p1.(G)5 |
| 17 | 48270168 | 48270172 | p1.(G)5 |
| 17 | 48271368 | 48271372 | p1.(G)5 |
| 17 | 48271815 | 48271819 | p1.(G)5 |
| 17 | 48272092 | 48272096 | p1.(G)5 |
| 17 | 48272164 | 48272168 | p1.(G)5 |
| 17 | 48272956 | 48272961 | p1.(G)6 |
| 17 | 48273272 | 48273277 | p1.(G)6 |
| 17 | 48273517 | 48273521 | p1.(G)5 |
| 17 | 48273722 | 48273726 | p1.(G)5 |
| 17 | 48274529 | 48274533 | p1.(C)5 |
| 17 | 48274591 | 48274595 | p1.(C)5 |
| 17 | 48276790 | 48276794 | p1.(G)5 |
| 17 | 53342786 | 53342790 | p1.(G)5 |
| 17 | 53342812 | 53342818 | p1.(T)7 |
| 17 | 53342825 | 53342830 | p1.(A)6 |
| 17 | 53342869 | 53342873 | p1.(C)5 |
| 17 | 53342941 | 53342945 | p1.(C)5 |
| 17 | 53345173 | 53345177 | p1.(C)5 |
| 17 | 53345267 | 53345271 | p1.(T)5 |
| 17 | 53345288 | 53345293 | p1.(C)6 |
| 17 | 53345355 | 53345359 | p1.(C)5 |
| 17 | 53398248 | 53398252 | p1.(T)5 |
| 17 | 55339499 | 55339506 | p1.(T)8 |
| 17 | 56435079 | 56435083 | p1.(G)5 |
| 17 | 56435094 | 56435098 | p1.(A)5 |
| 17 | 56435161 | 56435167 | p1.(C)7 |
| 17 | 56435205 | 56435209 | p1.(T)5 |
| 17 | 56435408 | 56435412 | p1.(G)5 |
| 17 | 56435458 | 56435462 | p1.(T)5 |
| 17 | 56435615 | 56435619 | p1.(G)5 |
| 17 | 56435669 | 56435673 | p1.(C)5 |
| 17 | 56435815 | 56435819 | p1.(G)5 |
| 17 | 56436028 | 56436032 | p1.(G)5 |
| 17 | 56436189 | 56436194 | p1.(A)6 |
| 17 | 56437531 | 56437535 | p1.(G)5 |
| 17 | 56437619 | 56437623 | p1.(A)5 |
| 17 | 56439919 | 56439923 | p1.(G)5 |
| 17 | 56440643 | 56440647 | p1.(G)5 |
| 17 | 56440773 | 56440777 | p1.(G)5 |
| 17 | 56448298 | 56448303 | p1.(G)6 |
| 17 | 57697479 | 57697483 | p1.(C)5 |
| 17 | 57721627 | 57721632 | p1.(T)6 |
| 17 | 57721719 | 57721723 | p1.(A)5 |
| 17 | 57724752 | 57724756 | p1.(T)5 |
| 17 | 57724777 | 57724781 | p1.(T)5 |
| 17 | 57733327 | 57733331 | p1.(T)5 |
| 17 | 57737894 | 57737898 | p1.(T)5 |
| 17 | 57741350 | 57741355 | p1.(A)6 |
| 17 | 57743453 | 57743459 | p1.(T)7 |
| 17 | 57744312 | 57744316 | p1.(T)5 |
| 17 | 57750998 | 57751004 | p1.(T)7 |
| 17 | 57752049 | 57752053 | p1.(T)5 |
| 17 | 57752130 | 57752135 | p1.(A)6 |
| 17 | 57752192 | 57752197 | p1.(A)6 |
| 17 | 57754304 | 57754309 | p1.(T)6 |
| 17 | 57758641 | 57758648 | p1.(T)8 |
| 17 | 57759977 | 57759985 | p1.(T)9 |
| 17 | 57760012 | 57760017 | p1.(A)6 |
| 17 | 57760793 | 57760797 | p1.(T)5 |
| 17 | 57761321 | 57761325 | p1.(T)5 |
| 17 | 57762403 | 57762413 | p1.(T)11 |
| 17 | 57762481 | 57762485 | p1.(A)5 |
| 17 | 57763011 | 57763015 | p1.(T)5 |
| 17 | 57763024 | 57763029 | p1.(A)6 |
| 17 | 59760685 | 59760689 | p1.(T)5 |
| 17 | 59760731 | 59760735 | p1.(T)5 |
| 17 | 59760764 | 59760768 | p1.(T)5 |
| 17 | 59760894 | 59760898 | p1.(A)5 |
| 17 | 59760967 | 59760973 | p1.(T)7 |
| 17 | 59761147 | 59761152 | p1.(T)6 |
| 17 | 59761414 | 59761425 | p4.(TTTG)3 |
| 17 | 59761460 | 59761465 | p1.(T)6 |
| 17 | 59763238 | 59763242 | p1.(T)5 |
| 17 | 59763432 | 59763436 | p1.(T)5 |
| 17 | 59763442 | 59763446 | p1.(T)5 |
| 17 | 59763533 | 59763537 | p1.(A)5 |
| 17 | 59821853 | 59821858 | p1.(T)6 |
| 17 | 59821941 | 59821945 | p1.(T)5 |
| 17 | 59857629 | 59857633 | p1.(T)5 |
| 17 | 59858268 | 59858273 | p1.(T)6 |
| 17 | 59858292 | 59858296 | p1.(T)5 |
| 17 | 59861645 | 59861649 | p1.(A)5 |
| 17 | 59861666 | 59861670 | p1.(A)5 |
| 17 | 59861749 | 59861754 | p1.(T)6 |
| 17 | 59861759 | 59861763 | p1.(T)5 |
| 17 | 59861776 | 59861780 | p1.(A)5 |
| 17 | 59878817 | 59878821 | p1.(A)5 |
| 17 | 59885829 | 59885833 | p1.(T)5 |
| 17 | 59886114 | 59886118 | p1.(G)5 |
| 17 | 59926603 | 59926608 | p1.(T)6 |
| 17 | 59934561 | 59934565 | p1.(T)5 |
| 17 | 59934595 | 59934599 | p1.(A)5 |
| 17 | 59937205 | 59937209 | p1.(T)5 |
| 17 | 62006838 | 62006842 | p1.(G)5 |
| 17 | 62009560 | 62009571 | p3.(AGC)4 |
| 17 | 62496298 | 62496302 | p1.(T)5 |
| 17 | 62498570 | 62498574 | p1.(T)5 |
| 17 | 62498610 | 62498614 | p1.(A)5 |
| 17 | 62499407 | 62499411 | p1.(T)5 |
| 17 | 62499421 | 62499425 | p1.(A)5 |
| 17 | 62500358 | 62500362 | p1.(A)5 |
| 17 | 62500830 | 62500834 | p1.(A)5 |
| 17 | 62500874 | 62500878 | p1.(T)5 |
| 17 | 62500969 | 62500976 | p1.(A)8 |
| 17 | 63010537 | 63010541 | p1.(T)5 |
| 17 | 63010693 | 63010697 | p1.(A)5 |
| 17 | 63010865 | 63010870 | p1.(T)6 |
| 17 | 63049740 | 63049744 | p1.(G)5 |
| 17 | 63049855 | 63049859 | p1.(A)5 |
| 17 | 63052468 | 63052477 | p2.(CG)5 |
| 17 | 63052715 | 63052732 | p3.(GCC)6 |
| 17 | 66511521 | 66511527 | p1.(T)7 |
| 17 | 66511720 | 66511724 | p1.(A)5 |
| 17 | 66520162 | 66520166 | p1.(T)5 |
| 17 | 66523968 | 66523972 | p1.(T)5 |
| 17 | 66523974 | 66523978 | p1.(T)5 |
| 17 | 73774661 | 73774665 | p1.(A)5 |
| 17 | 73775204 | 73775209 | p1.(G)6 |
| 17 | 74732279 | 74732284 | p1.(G)6 |
| 17 | 74732328 | 74732332 | p1.(G)5 |
| 17 | 74732908 | 74732912 | p1.(C)5 |
| 17 | 74732956 | 74732961 | p1.(G)6 |
| 17 | 74733223 | 74733228 | p1.(G)6 |
| 17 | 75398133 | 75398137 | p1.(T)5 |
| 17 | 75398318 | 75398322 | p1.(C)5 |
| 17 | 75398563 | 75398567 | p1.(C)5 |
| 17 | 75398615 | 75398619 | p1.(C)5 |
| 17 | 75398666 | 75398670 | p1.(C)5 |
| 17 | 75398668 | 75398682 | p5.(CCCAG)3 |
| 17 | 75398749 | 75398753 | p1.(C)5 |
| 17 | 75478268 | 75478272 | p1.(C)5 |
| 17 | 75484798 | 75484802 | p1.(C)5 |
| 17 | 75488741 | 75488745 | p1.(C)5 |
| 17 | 75495099 | 75495103 | p1.(G)5 |
| 17 | 75495225 | 75495229 | p1.(C)5 |
| 17 | 75495334 | 75495338 | p1.(C)5 |
| 17 | 75495452 | 75495456 | p1.(C)5 |
| 17 | 75495467 | 75495471 | p1.(C)5 |
| 17 | 75495473 | 75495477 | p1.(C)5 |
| 17 | 75495523 | 75495527 | p1.(A)5 |
| 17 | 75495548 | 75495552 | p1.(C)5 |
| 17 | 75495557 | 75495561 | p1.(C)5 |
| 17 | 76988740 | 76988744 | p1.(A)5 |
| 17 | 76993149 | 76993153 | p1.(C)5 |
| 17 | 76993313 | 76993317 | p1.(T)5 |
| 17 | 76993418 | 76993422 | p1.(G)5 |
| 17 | 76993428 | 76993432 | p1.(G)5 |
| 17 | 76993495 | 76993500 | p1.(G)6 |
| 17 | 76993507 | 76993511 | p1.(G)5 |
| 17 | 76993581 | 76993585 | p1.(G)5 |
| 17 | 76993593 | 76993597 | p1.(G)5 |
| 17 | 76993634 | 76993639 | p1.(C)6 |
| 17 | 78237521 | 78237525 | p1.(C)5 |
| 17 | 78237566 | 78237570 | p1.(C)5 |
| 17 | 78247117 | 78247121 | p1.(G)5 |
| 17 | 78261617 | 78261621 | p1.(A)5 |
| 17 | 78261638 | 78261642 | p1.(A)5 |
| 17 | 78261800 | 78261804 | p1.(C)5 |
| 17 | 78261868 | 78261872 | p1.(C)5 |
| 17 | 78261986 | 78261990 | p1.(G)5 |
| 17 | 78262019 | 78262024 | p1.(C)6 |
| 17 | 78263501 | 78263505 | p1.(A)5 |
| 17 | 78263581 | 78263585 | p1.(A)5 |
| 17 | 78263597 | 78263603 | p1.(A)7 |
| 17 | 78269384 | 78269389 | p1.(A)6 |
| 17 | 78269413 | 78269417 | p1.(A)5 |
| 17 | 78269426 | 78269430 | p1.(T)5 |
| 17 | 78269481 | 78269485 | p1.(T)5 |
| 17 | 78269493 | 78269498 | p1.(A)6 |
| 17 | 78286906 | 78286910 | p1.(T)5 |
| 17 | 78292981 | 78292985 | p1.(T)5 |
| 17 | 78302131 | 78302136 | p1.(A)6 |
| 17 | 78307893 | 78307897 | p1.(T)5 |
| 17 | 78307971 | 78307975 | p1.(A)5 |
| 17 | 78310007 | 78310011 | p1.(G)5 |
| 17 | 78310128 | 78310132 | p1.(A)5 |
| 17 | 78313098 | 78313102 | p1.(C)5 |
| 17 | 78313325 | 78313329 | p1.(C)5 |
| 17 | 78314019 | 78314024 | p1.(C)6 |
| 17 | 78316988 | 78316992 | p1.(A)5 |
| 17 | 78319284 | 78319288 | p1.(C)5 |
| 17 | 78319588 | 78319593 | p1.(T)6 |
| 17 | 78319849 | 78319853 | p1.(C)5 |
| 17 | 78320152 | 78320156 | p1.(A)5 |
| 17 | 78320327 | 78320331 | p1.(T)5 |
| 17 | 78320756 | 78320761 | p1.(C)6 |
| 17 | 78320764 | 78320768 | p1.(A)5 |
| 17 | 78320828 | 78320832 | p1.(T)5 |
| 17 | 78321274 | 78321278 | p1.(A)5 |
| 17 | 78321318 | 78321322 | p1.(G)5 |
| 17 | 78321341 | 78321345 | p1.(T)5 |
| 17 | 78321624 | 78321628 | p1.(C)5 |
| 17 | 78324139 | 78324143 | p1.(T)5 |
| 17 | 78324202 | 78324206 | p1.(T)5 |
| 17 | 78328279 | 78328283 | p1.(T)5 |
| 17 | 78343588 | 78343597 | p2.(TC)5 |
| 17 | 78345719 | 78345724 | p1.(A)6 |
| 17 | 78346883 | 78346887 | p1.(G)5 |
| 17 | 78349570 | 78349575 | p1.(C)6 |
| 17 | 78350086 | 78350092 | p1.(T)7 |
| 17 | 78350674 | 78350678 | p1.(T)5 |
| 17 | 78354713 | 78354717 | p1.(C)5 |
| 17 | 78355338 | 78355342 | p1.(C)5 |
| 17 | 78356768 | 78356774 | p1.(T)7 |
| 17 | 78357681 | 78357685 | p1.(A)5 |
| 17 | 78360206 | 78360210 | p1.(A)5 |
| 17 | 78363955 | 78363959 | p1.(C)5 |
| 17 | 78367137 | 78367141 | p1.(T)5 |
| 17 | 78519367 | 78519371 | p1.(T)5 |
| 17 | 78519409 | 78519413 | p1.(C)5 |
| 17 | 78519415 | 78519419 | p1.(C)5 |
| 17 | 78519421 | 78519425 | p1.(C)5 |
| 17 | 78519468 | 78519472 | p1.(G)5 |
| 17 | 78519523 | 78519527 | p1.(A)5 |
| 17 | 78727867 | 78727871 | p1.(A)5 |
| 17 | 78727961 | 78727965 | p1.(C)5 |
| 17 | 78796094 | 78796098 | p1.(C)5 |
| 17 | 78796934 | 78796939 | p1.(T)6 |
| 17 | 78829315 | 78829319 | p1.(T)5 |
| 17 | 78831583 | 78831587 | p1.(C)5 |
| 17 | 78857290 | 78857295 | p1.(C)6 |
| 17 | 78866568 | 78866572 | p1.(C)5 |
| 17 | 78867528 | 78867532 | p1.(C)5 |
| 17 | 78882599 | 78882608 | p2.(TC)5 |
| 17 | 78896582 | 78896586 | p1.(C)5 |
| 17 | 78897434 | 78897438 | p1.(C)5 |
| 17 | 78936354 | 78936358 | p1.(C)5 |
| 17 | 79937053 | 79937057 | p1.(C)5 |
| 17 | 79953922 | 79953926 | p1.(G)5 |
| 17 | 79954508 | 79954512 | p1.(C)5 |
| 17 | 79954531 | 79954535 | p1.(G)5 |
| 17 | 79954545 | 79954550 | p1.(G)6 |
| 17 | 79967075 | 79967079 | p1.(C)5 |
| 17 | 79969420 | 79969424 | p1.(G)5 |
| 17 | 79969487 | 79969492 | p1.(T)6 |
| 17 | 79970120 | 79970124 | p1.(A)5 |
| 17 | 79974898 | 79974902 | p1.(G)5 |
| 17 | 79974910 | 79974915 | p1.(C)6 |
| 18 | 22669448 | 22669452 | p1.(A)5 |
| 18 | 22804478 | 22804483 | p1.(C)6 |
| 18 | 22804693 | 22804697 | p1.(T)5 |
| 18 | 22804871 | 22804875 | p1.(A)5 |
| 18 | 22805120 | 22805124 | p1.(T)5 |
| 18 | 22805389 | 22805393 | p1.(T)5 |
| 18 | 22805404 | 22805408 | p1.(T)5 |
| 18 | 22805689 | 22805693 | p1.(A)5 |
| 18 | 22805919 | 22805923 | p1.(T)5 |
| 18 | 22806063 | 22806067 | p1.(G)5 |
| 18 | 22806122 | 22806126 | p1.(T)5 |
| 18 | 22807093 | 22807097 | p1.(T)5 |
| 18 | 22902155 | 22902159 | p1.(A)5 |
| 18 | 22932034 | 22932038 | p1.(C)5 |
| 18 | 22932062 | 22932066 | p1.(G)5 |
| 18 | 22932192 | 22932196 | p1.(G)5 |
| 18 | 23615012 | 23615023 | p3.(CTG)4 |
| 18 | 23618627 | 23618631 | p1.(A)5 |
| 18 | 23632818 | 23632822 | p1.(A)5 |
| 18 | 23637543 | 23637547 | p1.(A)5 |
| 18 | 23637600 | 23637604 | p1.(C)5 |
| 18 | 23637648 | 23637652 | p1.(G)5 |
| 18 | 23670491 | 23670495 | p1.(C)5 |
| 18 | 23670590 | 23670594 | p1.(G)5 |
| 18 | 42281351 | 42281355 | p1.(G)5 |
| 18 | 42281390 | 42281394 | p1.(C)5 |
| 18 | 42281657 | 42281661 | p1.(C)5 |
| 18 | 42281746 | 42281751 | p1.(A)6 |
| 18 | 42281789 | 42281793 | p1.(A)5 |
| 18 | 42529940 | 42529945 | p1.(A)6 |
| 18 | 42530278 | 42530282 | p1.(C)5 |
| 18 | 42530308 | 42530312 | p1.(A)5 |
| 18 | 42530320 | 42530325 | p1.(A)6 |
| 18 | 42530560 | 42530566 | p1.(A)7 |
| 18 | 42530729 | 42530733 | p1.(C)5 |
| 18 | 42530977 | 42530981 | p1.(C)5 |
| 18 | 42531055 | 42531059 | p1.(A)5 |
| 18 | 42531328 | 42531332 | p1.(A)5 |
| 18 | 42531388 | 42531392 | p1.(C)5 |
| 18 | 42531514 | 42531518 | p1.(C)5 |
| 18 | 42531699 | 42531703 | p1.(T)5 |
| 18 | 42532259 | 42532263 | p1.(T)5 |
| 18 | 42532374 | 42532378 | p1.(A)5 |
| 18 | 42532404 | 42532409 | p1.(T)6 |
| 18 | 42532823 | 42532827 | p1.(T)5 |
| 18 | 42533045 | 42533049 | p1.(A)5 |
| 18 | 42618442 | 42618447 | p1.(T)6 |
| 18 | 42643201 | 42643205 | p1.(A)5 |
| 18 | 42643299 | 42643303 | p1.(A)5 |
| 18 | 45372131 | 45372135 | p1.(A)5 |
| 18 | 45391420 | 45391427 | p1.(T)8 |
| 18 | 45394686 | 45394690 | p1.(A)5 |
| 18 | 45394832 | 45394836 | p1.(A)5 |
| 18 | 48573403 | 48573407 | p1.(A)5 |
| 18 | 48573524 | 48573528 | p1.(A)5 |
| 18 | 48573564 | 48573569 | p1.(A)6 |
| 18 | 48575047 | 48575051 | p1.(T)5 |
| 18 | 48575122 | 48575126 | p1.(A)5 |
| 18 | 48584514 | 48584519 | p1.(G)6 |
| 18 | 48584703 | 48584707 | p1.(T)5 |
| 18 | 48602994 | 48602999 | p1.(T)6 |
| 18 | 56348485 | 56348489 | p1.(A)5 |
| 18 | 56348561 | 56348566 | p1.(C)6 |
| 18 | 56400758 | 56400762 | p1.(A)5 |
| 18 | 56411562 | 56411566 | p1.(T)5 |
| 18 | 56412883 | 56412893 | p1.(T)11 |
| 18 | 56413013 | 56413017 | p1.(A)5 |
| 18 | 56415067 | 56415071 | p1.(A)5 |
| 18 | 56415083 | 56415087 | p1.(T)5 |
| 18 | 60796001 | 60796005 | p1.(G)5 |
| 18 | 60985465 | 60985469 | p1.(C)5 |
| 18 | 60985666 | 60985670 | p1.(G)5 |
| 18 | 60985768 | 60985772 | p1.(G)5 |
| 18 | 60985776 | 60985785 | p1.(C)5(G)5 |
| 18 | 60999042 | 60999046 | p1.(T)5 |
| 18 | 61022426 | 61022430 | p1.(T)5 |
| 18 | 61022537 | 61022541 | p1.(A)5 |
| 19 | 680323 | 680328 | p1.(G)6 |
| 19 | 680496 | 680500 | p1.(G)5 |
| 19 | 1207065 | 1207069 | p1.(G)5 |
| 19 | 1207077 | 1207081 | p1.(G)5 |
| 19 | 1207153 | 1207164 | p3.(AAG)4 |
| 19 | 1218447 | 1218451 | p1.(A)5 |
| 19 | 1221314 | 1221319 | p1.(C)6 |
| 19 | 1226573 | 1226577 | p1.(C)5 |
| 19 | 1611771 | 1611775 | p1.(G)5 |
| 19 | 1615273 | 1615277 | p1.(C)5 |
| 19 | 1615487 | 1615492 | p1.(G)6 |
| 19 | 1615698 | 1615703 | p1.(G)6 |
| 19 | 1619152 | 1619163 | p4.(CTGG)3 |
| 19 | 1619792 | 1619796 | p1.(C)5 |
| 19 | 1619821 | 1619825 | p1.(G)5 |
| 19 | 1620979 | 1620984 | p1.(G)6 |
| 19 | 1620992 | 1620996 | p1.(G)5 |
| 19 | 1621901 | 1621905 | p1.(G)5 |
| 19 | 1621972 | 1621977 | p1.(G)6 |
| 19 | 1622147 | 1622151 | p1.(C)5 |
| 19 | 1622183 | 1622187 | p1.(G)5 |
| 19 | 1622325 | 1622329 | p1.(G)5 |
| 19 | 1622346 | 1622350 | p1.(G)5 |
| 19 | 1646432 | 1646446 | p5.(GGGAG)3 |
| 19 | 2164138 | 2164142 | p1.(C)5 |
| 19 | 2164171 | 2164180 | p2.(CG)5 |
| 19 | 2164186 | 2164190 | p1.(G)5 |
| 19 | 2164222 | 2164226 | p1.(G)5 |
| 19 | 2189723 | 2189727 | p1.(T)5 |
| 19 | 2191141 | 2191145 | p1.(C)5 |
| 19 | 2194555 | 2194560 | p1.(A)6 |
| 19 | 2210863 | 2210867 | p1.(C)5 |
| 19 | 2211158 | 2211162 | p1.(C)5 |
| 19 | 2211795 | 2211800 | p1.(C)6 |
| 19 | 2213626 | 2213630 | p1.(A)5 |
| 19 | 2220114 | 2220118 | p1.(C)5 |
| 19 | 2220213 | 2220217 | p1.(C)5 |
| 19 | 2222089 | 2222093 | p1.(T)5 |
| 19 | 2222129 | 2222133 | p1.(C)5 |
| 19 | 2222363 | 2222367 | p1.(C)5 |
| 19 | 2222479 | 2222483 | p1.(C)5 |
| 19 | 2223376 | 2223381 | p1.(C)6 |
| 19 | 2225372 | 2225378 | p1.(T)7 |
| 19 | 2225396 | 2225400 | p1.(A)5 |
| 19 | 2225430 | 2225434 | p1.(C)5 |
| 19 | 2226423 | 2226427 | p1.(G)5 |
| 19 | 2226478 | 2226482 | p1.(G)5 |
| 19 | 3094772 | 3094783 | p3.(GCT)4 |
| 19 | 3110140 | 3110144 | p1.(C)5 |
| 19 | 3118934 | 3118939 | p1.(G)6 |
| 19 | 3120980 | 3120984 | p1.(C)5 |
| 19 | 4094499 | 4094503 | p1.(G)5 |
| 19 | 4095412 | 4095416 | p1.(G)5 |
| 19 | 4099215 | 4099220 | p1.(G)6 |
| 19 | 4099312 | 4099317 | p1.(G)6 |
| 19 | 4123888 | 4123899 | p3.(CGG)4 |
| 19 | 4361796 | 4361800 | p1.(G)5 |
| 19 | 4362619 | 4362623 | p1.(G)5 |
| 19 | 4362641 | 4362645 | p1.(C)5 |
| 19 | 4364123 | 4364127 | p1.(G)5 |
| 19 | 6213029 | 6213033 | p1.(G)5 |
| 19 | 6213336 | 6213341 | p1.(G)6 |
| 19 | 6213427 | 6213433 | p1.(G)7 |
| 19 | 6213728 | 6213734 | p1.(C)7 |
| 19 | 6222187 | 6222191 | p1.(T)5 |
| 19 | 6222272 | 6222289 | p3.(AGG)6 |
| 19 | 6222406 | 6222410 | p1.(G)5 |
| 19 | 6222412 | 6222416 | p1.(G)5 |
| 19 | 6222418 | 6222422 | p1.(G)5 |
| 19 | 6222424 | 6222428 | p1.(G)5 |
| 19 | 6222438 | 6222442 | p1.(G)5 |
| 19 | 6262243 | 6262247 | p1.(T)5 |
| 19 | 6262305 | 6262310 | p1.(G)6 |
| 19 | 6262324 | 6262328 | p1.(A)5 |
| 19 | 10597273 | 10597277 | p1.(A)5 |
| 19 | 10597303 | 10597307 | p1.(T)5 |
| 19 | 10599929 | 10599933 | p1.(G)5 |
| 19 | 10600005 | 10600009 | p1.(C)5 |
| 19 | 10600425 | 10600430 | p1.(C)6 |
| 19 | 10602320 | 10602324 | p1.(C)5 |
| 19 | 10602354 | 10602358 | p1.(G)5 |
| 19 | 10610631 | 10610635 | p1.(C)5 |
| 19 | 10828903 | 10828907 | p1.(G)5 |
| 19 | 10870401 | 10870407 | p1.(C)7 |
| 19 | 10870480 | 10870484 | p1.(A)5 |
| 19 | 10883166 | 10883170 | p1.(T)5 |
| 19 | 10883184 | 10883188 | p1.(A)5 |
| 19 | 10904475 | 10904479 | p1.(G)5 |
| 19 | 10904538 | 10904543 | p1.(C)6 |
| 19 | 10906780 | 10906784 | p1.(A)5 |
| 19 | 10909155 | 10909159 | p1.(C)5 |
| 19 | 10909173 | 10909177 | p1.(C)5 |
| 19 | 10912957 | 10912961 | p1.(C)5 |
| 19 | 10922933 | 10922937 | p1.(C)5 |
| 19 | 10934453 | 10934459 | p1.(C)7 |
| 19 | 10935777 | 10935781 | p1.(C)5 |
| 19 | 10939898 | 10939902 | p1.(C)5 |
| 19 | 10940839 | 10940844 | p1.(C)6 |
| 19 | 10940852 | 10940856 | p1.(C)5 |
| 19 | 10940882 | 10940888 | p1.(C)7 |
| 19 | 10940905 | 10940909 | p1.(G)5 |
| 19 | 10940930 | 10940934 | p1.(C)5 |
| 19 | 10941025 | 10941030 | p1.(C)6 |
| 19 | 10941038 | 10941042 | p1.(C)5 |
| 19 | 10941661 | 10941665 | p1.(C)5 |
| 19 | 10941731 | 10941736 | p1.(G)6 |
| 19 | 10941746 | 10941752 | p1.(G)7 |
| 19 | 11096027 | 11096031 | p1.(G)5 |
| 19 | 11096048 | 11096052 | p1.(C)5 |
| 19 | 11096983 | 11096987 | p1.(C)5 |
| 19 | 11097084 | 11097088 | p1.(G)5 |
| 19 | 11097604 | 11097608 | p1.(C)5 |
| 19 | 11097625 | 11097630 | p1.(C)6 |
| 19 | 11098363 | 11098367 | p1.(C)5 |
| 19 | 11098392 | 11098396 | p1.(C)5 |
| 19 | 11098417 | 11098421 | p1.(C)5 |
| 19 | 11098425 | 11098429 | p1.(C)5 |
| 19 | 11098477 | 11098481 | p1.(C)5 |
| 19 | 11098540 | 11098544 | p1.(C)5 |
| 19 | 11107033 | 11107037 | p1.(A)5 |
| 19 | 11107042 | 11107046 | p1.(A)5 |
| 19 | 11107048 | 11107052 | p1.(A)5 |
| 19 | 11113697 | 11113701 | p1.(T)5 |
| 19 | 11113790 | 11113794 | p1.(C)5 |
| 19 | 11114006 | 11114011 | p1.(T)6 |
| 19 | 11118576 | 11118587 | p3.(AGG)4 |
| 19 | 11118633 | 11118644 | p3.(AGA)4 |
| 19 | 11121216 | 11121220 | p1.(G)5 |
| 19 | 11129671 | 11129675 | p1.(C)5 |
| 19 | 11130296 | 11130300 | p1.(C)5 |
| 19 | 11134182 | 11134186 | p1.(G)5 |
| 19 | 11141498 | 11141503 | p1.(G)6 |
| 19 | 11141541 | 11141545 | p1.(T)5 |
| 19 | 11143959 | 11143963 | p1.(C)5 |
| 19 | 11145716 | 11145730 | p3.(GAG)5 |
| 19 | 13049479 | 13049483 | p1.(C)5 |
| 19 | 13049937 | 13049941 | p1.(C)5 |
| 19 | 13050364 | 13050368 | p1.(G)5 |
| 19 | 13050970 | 13050974 | p1.(G)5 |
| 19 | 13051347 | 13051352 | p1.(C)6 |
| 19 | 13051439 | 13051443 | p1.(C)5 |
| 19 | 13210215 | 13210219 | p1.(G)5 |
| 19 | 13211729 | 13211733 | p1.(G)5 |
| 19 | 13211774 | 13211778 | p1.(G)5 |
| 19 | 13211824 | 13211829 | p1.(G)6 |
| 19 | 13211867 | 13211871 | p1.(G)5 |
| 19 | 13211891 | 13211895 | p1.(G)5 |
| 19 | 13211914 | 13211918 | p1.(G)5 |
| 19 | 15366368 | 15366372 | p1.(G)5 |
| 19 | 15367010 | 15367014 | p1.(T)5 |
| 19 | 15367902 | 15367906 | p1.(G)5 |
| 19 | 15375495 | 15375499 | p1.(G)5 |
| 19 | 15375536 | 15375540 | p1.(G)5 |
| 19 | 15376235 | 15376239 | p1.(G)5 |
| 19 | 15376248 | 15376254 | p1.(G)7 |
| 19 | 15376262 | 15376266 | p1.(G)5 |
| 19 | 15378305 | 15378310 | p1.(T)6 |
| 19 | 15383768 | 15383772 | p1.(G)5 |
| 19 | 15383774 | 15383779 | p1.(G)6 |
| 19 | 15383856 | 15383860 | p1.(C)5 |
| 19 | 16192832 | 16192836 | p1.(A)5 |
| 19 | 17937615 | 17937619 | p1.(C)5 |
| 19 | 17937647 | 17937651 | p1.(G)5 |
| 19 | 17941420 | 17941424 | p1.(G)5 |
| 19 | 17942212 | 17942223 | p4.(GCGG)3 |
| 19 | 17942472 | 17942476 | p1.(C)5 |
| 19 | 17943520 | 17943524 | p1.(G)5 |
| 19 | 17943746 | 17943750 | p1.(G)5 |
| 19 | 17945489 | 17945493 | p1.(G)5 |
| 19 | 17945781 | 17945785 | p1.(G)5 |
| 19 | 17945961 | 17945965 | p1.(C)5 |
| 19 | 17946029 | 17946033 | p1.(G)5 |
| 19 | 17946828 | 17946832 | p1.(C)5 |
| 19 | 17950344 | 17950349 | p1.(C)6 |
| 19 | 17950466 | 17950470 | p1.(G)5 |
| 19 | 17951077 | 17951081 | p1.(G)5 |
| 19 | 17951115 | 17951119 | p1.(C)5 |
| 19 | 17951155 | 17951159 | p1.(G)5 |
| 19 | 17953399 | 17953403 | p1.(G)5 |
| 19 | 17953984 | 17953989 | p1.(G)6 |
| 19 | 17954643 | 17954648 | p1.(G)6 |
| 19 | 17955112 | 17955118 | p1.(G)7 |
| 19 | 18266938 | 18266952 | p5.(GGCCC)3 |
| 19 | 18266965 | 18266969 | p1.(C)5 |
| 19 | 18271271 | 18271275 | p1.(C)5 |
| 19 | 18271331 | 18271335 | p1.(C)5 |
| 19 | 18271722 | 18271726 | p1.(C)5 |
| 19 | 18271957 | 18271961 | p1.(C)5 |
| 19 | 18272767 | 18272771 | p1.(C)5 |
| 19 | 18272855 | 18272859 | p1.(C)5 |
| 19 | 18273005 | 18273009 | p1.(C)5 |
| 19 | 18273026 | 18273031 | p1.(C)6 |
| 19 | 18273105 | 18273110 | p1.(G)6 |
| 19 | 18273319 | 18273323 | p1.(G)5 |
| 19 | 18273770 | 18273774 | p1.(C)5 |
| 19 | 18276957 | 18276961 | p1.(C)5 |
| 19 | 18279340 | 18279344 | p1.(A)5 |
| 19 | 18279523 | 18279528 | p1.(C)6 |
| 19 | 18279888 | 18279892 | p1.(C)5 |
| 19 | 18555523 | 18555527 | p1.(C)5 |
| 19 | 18557106 | 18557112 | p1.(G)7 |
| 19 | 18561309 | 18561313 | p1.(G)5 |
| 19 | 18561489 | 18561493 | p1.(G)5 |
| 19 | 18561563 | 18561567 | p1.(G)5 |
| 19 | 18561580 | 18561584 | p1.(G)5 |
| 19 | 18561595 | 18561599 | p1.(G)5 |
| 19 | 18561752 | 18561756 | p1.(G)5 |
| 19 | 18569046 | 18569050 | p1.(C)5 |
| 19 | 18572536 | 18572540 | p1.(C)5 |
| 19 | 18572589 | 18572593 | p1.(G)5 |
| 19 | 18576644 | 18576648 | p1.(G)5 |
| 19 | 18576713 | 18576717 | p1.(G)5 |
| 19 | 18632881 | 18632885 | p1.(C)5 |
| 19 | 18632889 | 18632893 | p1.(C)5 |
| 19 | 18794474 | 18794497 | p3.(GGA)8 |
| 19 | 18794643 | 18794647 | p1.(G)5 |
| 19 | 18856626 | 18856630 | p1.(C)5 |
| 19 | 18856634 | 18856638 | p1.(C)5 |
| 19 | 18864360 | 18864364 | p1.(A)5 |
| 19 | 18870879 | 18870884 | p1.(G)6 |
| 19 | 18876263 | 18876267 | p1.(C)5 |
| 19 | 18879460 | 18879464 | p1.(C)5 |
| 19 | 18879471 | 18879475 | p1.(C)5 |
| 19 | 18879514 | 18879518 | p1.(C)5 |
| 19 | 18879546 | 18879550 | p1.(C)5 |
| 19 | 18879554 | 18879560 | p1.(C)7 |
| 19 | 18887974 | 18887978 | p1.(C)5 |
| 19 | 18887993 | 18888000 | p1.(C)8 |
| 19 | 18888096 | 18888100 | p1.(C)5 |
| 19 | 19256495 | 19256500 | p1.(G)6 |
| 19 | 19256727 | 19256736 | p1.(C)5(G)5 |
| 19 | 19256769 | 19256775 | p1.(G)7 |
| 19 | 19256799 | 19256803 | p1.(G)5 |
| 19 | 19256826 | 19256830 | p1.(C)5 |
| 19 | 19256837 | 19256841 | p1.(G)5 |
| 19 | 19257564 | 19257568 | p1.(C)5 |
| 19 | 19257630 | 19257634 | p1.(G)5 |
| 19 | 19257847 | 19257851 | p1.(G)5 |
| 19 | 19257861 | 19257865 | p1.(G)5 |
| 19 | 19258538 | 19258542 | p1.(C)5 |
| 19 | 19261529 | 19261535 | p1.(T)7 |
| 19 | 30303876 | 30303880 | p1.(T)5 |
| 19 | 30308412 | 30308416 | p1.(A)5 |
| 19 | 30311698 | 30311702 | p1.(A)5 |
| 19 | 33793253 | 33793258 | p1.(G)6 |
| 19 | 33793280 | 33793284 | p1.(G)5 |
| 19 | 33793321 | 33793325 | p1.(G)5 |
| 19 | 40739717 | 40739727 | p1.(G)5(A)6 |
| 19 | 40739811 | 40739815 | p1.(G)5 |
| 19 | 40740960 | 40740964 | p1.(G)5 |
| 19 | 40741245 | 40741250 | p1.(C)6 |
| 19 | 40741967 | 40741971 | p1.(C)5 |
| 19 | 40744816 | 40744820 | p1.(C)5 |
| 19 | 40744883 | 40744887 | p1.(G)5 |
| 19 | 40748501 | 40748505 | p1.(G)5 |
| 19 | 40762855 | 40762860 | p1.(G)6 |
| 19 | 41725248 | 41725252 | p1.(C)5 |
| 19 | 41725254 | 41725258 | p1.(C)5 |
| 19 | 41725374 | 41725378 | p1.(C)5 |
| 19 | 41726645 | 41726650 | p1.(C)6 |
| 19 | 41727836 | 41727840 | p1.(C)5 |
| 19 | 41727868 | 41727872 | p1.(C)5 |
| 19 | 41727909 | 41727913 | p1.(C)5 |
| 19 | 41727936 | 41727940 | p1.(C)5 |
| 19 | 41736918 | 41736922 | p1.(G)5 |
| 19 | 41737092 | 41737096 | p1.(C)5 |
| 19 | 41737179 | 41737183 | p1.(C)5 |
| 19 | 41743896 | 41743901 | p1.(C)6 |
| 19 | 41743933 | 41743939 | p1.(C)7 |
| 19 | 41744385 | 41744390 | p1.(C)6 |
| 19 | 41745116 | 41745120 | p1.(G)5 |
| 19 | 41748777 | 41748781 | p1.(C)5 |
| 19 | 41749512 | 41749518 | p1.(C)7 |
| 19 | 41754521 | 41754525 | p1.(C)5 |
| 19 | 41754638 | 41754643 | p1.(C)6 |
| 19 | 41758740 | 41758745 | p1.(G)6 |
| 19 | 41765621 | 41765625 | p1.(C)5 |
| 19 | 41765647 | 41765652 | p1.(C)6 |
| 19 | 42381381 | 42381385 | p1.(G)5 |
| 19 | 42383118 | 42383122 | p1.(G)5 |
| 19 | 42383209 | 42383213 | p1.(C)5 |
| 19 | 42383281 | 42383285 | p1.(G)5 |
| 19 | 42383610 | 42383615 | p1.(C)6 |
| 19 | 42383633 | 42383637 | p1.(G)5 |
| 19 | 42383729 | 42383733 | p1.(C)5 |
| 19 | 42385070 | 42385074 | p1.(C)5 |
| 19 | 42788840 | 42788845 | p1.(C)6 |
| 19 | 42791229 | 42791233 | p1.(C)5 |
| 19 | 42791268 | 42791272 | p1.(C)5 |
| 19 | 42791282 | 42791286 | p1.(G)5 |
| 19 | 42791339 | 42791344 | p1.(C)6 |
| 19 | 42792124 | 42792128 | p1.(G)5 |
| 19 | 42793141 | 42793145 | p1.(G)5 |
| 19 | 42793444 | 42793448 | p1.(C)5 |
| 19 | 42794441 | 42794446 | p1.(C)6 |
| 19 | 42794460 | 42794464 | p1.(C)5 |
| 19 | 42794469 | 42794473 | p1.(C)5 |
| 19 | 42794627 | 42794631 | p1.(C)5 |
| 19 | 42794637 | 42794641 | p1.(C)5 |
| 19 | 42794643 | 42794647 | p1.(C)5 |
| 19 | 42794655 | 42794659 | p1.(G)5 |
| 19 | 42794820 | 42794824 | p1.(C)5 |
| 19 | 42794869 | 42794873 | p1.(C)5 |
| 19 | 42795075 | 42795079 | p1.(G)5 |
| 19 | 42795243 | 42795247 | p1.(C)5 |
| 19 | 42795269 | 42795273 | p1.(G)5 |
| 19 | 42795378 | 42795382 | p1.(G)5 |
| 19 | 42795609 | 42795614 | p1.(C)6 |
| 19 | 42795739 | 42795743 | p1.(C)5 |
| 19 | 42795886 | 42795891 | p1.(C)6 |
| 19 | 42796799 | 42796803 | p1.(C)5 |
| 19 | 42796883 | 42796889 | p1.(C)7 |
| 19 | 42797135 | 42797139 | p1.(C)5 |
| 19 | 42797165 | 42797170 | p1.(C)6 |
| 19 | 42797376 | 42797381 | p1.(C)6 |
| 19 | 42797386 | 42797390 | p1.(C)5 |
| 19 | 42797951 | 42797955 | p1.(C)5 |
| 19 | 42798164 | 42798168 | p1.(C)5 |
| 19 | 42798406 | 42798410 | p1.(C)5 |
| 19 | 42798428 | 42798432 | p1.(G)5 |
| 19 | 42799098 | 42799102 | p1.(C)5 |
| 19 | 42799131 | 42799136 | p1.(C)6 |
| 19 | 45252212 | 45252216 | p1.(C)5 |
| 19 | 45252261 | 45252267 | p1.(C)7 |
| 19 | 45252312 | 45252316 | p1.(C)5 |
| 19 | 45254584 | 45254588 | p1.(C)5 |
| 19 | 45259476 | 45259480 | p1.(C)5 |
| 19 | 45259482 | 45259486 | p1.(C)5 |
| 19 | 45259558 | 45259563 | p1.(G)6 |
| 19 | 45261997 | 45262001 | p1.(G)5 |
| 19 | 45262066 | 45262070 | p1.(C)5 |
| 19 | 45262726 | 45262731 | p1.(C)6 |
| 19 | 45262737 | 45262742 | p1.(C)6 |
| 19 | 45262770 | 45262781 | p4.(CTTC)3 |
| 19 | 45262843 | 45262848 | p1.(C)6 |
| 19 | 45262873 | 45262877 | p1.(G)5 |
| 19 | 45262880 | 45262886 | p1.(G)7 |
| 19 | 45281211 | 45281215 | p1.(G)5 |
| 19 | 45281290 | 45281294 | p1.(C)5 |
| 19 | 45281309 | 45281313 | p1.(C)5 |
| 19 | 45281399 | 45281403 | p1.(G)5 |
| 19 | 45284229 | 45284233 | p1.(G)5 |
| 19 | 45284269 | 45284273 | p1.(C)5 |
| 19 | 45293254 | 45293258 | p1.(C)5 |
| 19 | 45296724 | 45296728 | p1.(C)5 |
| 19 | 45297485 | 45297489 | p1.(C)5 |
| 19 | 45297503 | 45297508 | p1.(C)6 |
| 19 | 45855584 | 45855588 | p1.(C)5 |
| 19 | 45867598 | 45867602 | p1.(G)5 |
| 19 | 45867672 | 45867677 | p1.(C)6 |
| 19 | 45867808 | 45867813 | p1.(G)6 |
| 19 | 45868419 | 45868423 | p1.(G)5 |
| 19 | 45871907 | 45871911 | p1.(T)5 |
| 19 | 45873782 | 45873786 | p1.(C)5 |
| 19 | 45916842 | 45916847 | p1.(T)6 |
| 19 | 45916899 | 45916903 | p1.(C)5 |
| 19 | 45922461 | 45922465 | p1.(G)5 |
| 19 | 45926605 | 45926609 | p1.(C)5 |
| 19 | 51376683 | 51376688 | p1.(C)6 |
| 19 | 51376784 | 51376789 | p1.(G)6 |
| 19 | 51377965 | 51377969 | p1.(C)5 |
| 19 | 51378081 | 51378085 | p1.(G)5 |
| 19 | 51378098 | 51378102 | p1.(C)5 |
| 19 | 51381669 | 51381673 | p1.(G)5 |
| 19 | 52714707 | 52714711 | p1.(C)5 |
| 19 | 52715968 | 52715972 | p1.(C)5 |
| 19 | 52716002 | 52716006 | p1.(G)5 |
| 19 | 52725345 | 52725349 | p1.(C)5 |
| 19 | 52729280 | 52729284 | p1.(C)5 |
| 19 | 52729310 | 52729315 | p1.(G)6 |
| 19 | 54079940 | 54079945 | p1.(T)6 |
| 19 | 54079990 | 54079996 | p1.(A)7 |
| 19 | 54080347 | 54080352 | p1.(A)6 |
| 19 | 54080449 | 54080453 | p1.(A)5 |
| 19 | 54611426 | 54611430 | p1.(G)5 |
| 19 | 54611448 | 54611452 | p1.(G)5 |
| 19 | 54611522 | 54611526 | p1.(G)5 |
| 19 | 54611710 | 54611714 | p1.(G)5 |
| 19 | 54647257 | 54647261 | p1.(G)5 |
| 19 | 54651892 | 54651896 | p1.(A)5 |
| 19 | 54651966 | 54651971 | p1.(C)6 |
| 19 | 54652022 | 54652026 | p1.(C)5 |
| 19 | 54652033 | 54652037 | p1.(C)5 |
| 19 | 54652156 | 54652161 | p1.(C)6 |
| 19 | 54652408 | 54652412 | p1.(G)5 |
| 19 | 54652440 | 54652445 | p1.(C)6 |
| 19 | 54652452 | 54652456 | p1.(C)5 |
| 19 | 54653322 | 54653326 | p1.(G)5 |
| 19 | 54653329 | 54653333 | p1.(G)5 |
| 19 | 54653362 | 54653366 | p1.(G)5 |
| 19 | 54656613 | 54656617 | p1.(C)5 |
| 19 | 54656635 | 54656640 | p1.(C)6 |
| 19 | 54656659 | 54656663 | p1.(C)5 |
| 19 | 54657445 | 54657449 | p1.(C)5 |
| 19 | 54659036 | 54659040 | p1.(C)5 |
| 19 | 54659168 | 54659172 | p1.(C)5 |
| 19 | 54659175 | 54659179 | p1.(C)5 |
| 19 | 54659190 | 54659194 | p1.(C)5 |
| 20 | 31017156 | 31017160 | p1.(A)5 |
| 20 | 31017747 | 31017758 | p3.(CAG)4 |
| 20 | 31019113 | 31019117 | p1.(A)5 |
| 20 | 31019437 | 31019442 | p1.(T)6 |
| 20 | 31021277 | 31021282 | p1.(A)6 |
| 20 | 31022224 | 31022230 | p1.(T)7 |
| 20 | 31022297 | 31022301 | p1.(C)5 |
| 20 | 31022442 | 31022449 | p1.(G)8 |
| 20 | 31023046 | 31023050 | p1.(C)5 |
| 20 | 31023983 | 31023988 | p1.(A)6 |
| 20 | 31024426 | 31024430 | p1.(T)5 |
| 20 | 31024510 | 31024514 | p1.(T)5 |
| 20 | 31024637 | 31024642 | p1.(G)6 |
| 20 | 36012536 | 36012545 | p2.(TC)5 |
| 20 | 36012641 | 36012645 | p1.(G)5 |
| 20 | 36012666 | 36012670 | p1.(C)5 |
| 20 | 36012720 | 36012724 | p1.(C)5 |
| 20 | 36014585 | 36014589 | p1.(C)5 |
| 20 | 36022570 | 36022574 | p1.(C)5 |
| 20 | 36030027 | 36030031 | p1.(G)5 |
| 20 | 39316497 | 39316501 | p1.(G)5 |
| 20 | 39317087 | 39317098 | p3.(TGG)4 |
| 20 | 39658062 | 39658066 | p1.(T)5 |
| 20 | 39704915 | 39704919 | p1.(A)5 |
| 20 | 39706242 | 39706246 | p1.(A)5 |
| 20 | 39725848 | 39725853 | p1.(T)6 |
| 20 | 39725912 | 39725916 | p1.(A)5 |
| 20 | 39725955 | 39725959 | p1.(A)5 |
| 20 | 39728715 | 39728719 | p1.(A)5 |
| 20 | 39741464 | 39741469 | p1.(A)6 |
| 20 | 39742590 | 39742601 | p4.(TTTC)3 |
| 20 | 39744012 | 39744016 | p1.(T)5 |
| 20 | 39744063 | 39744067 | p1.(T)5 |
| 20 | 39746897 | 39746901 | p1.(A)5 |
| 20 | 43955925 | 43955929 | p1.(G)5 |
| 20 | 43964533 | 43964537 | p1.(G)5 |
| 20 | 43977016 | 43977020 | p1.(G)5 |
| 20 | 52188261 | 52188271 | p1.(T)6(C)5 |
| 20 | 52188288 | 52188294 | p1.(T)7 |
| 20 | 52188387 | 52188391 | p1.(T)5 |
| 20 | 52188400 | 52188409 | p1.(A)10 |
| 20 | 52192548 | 52192552 | p1.(T)5 |
| 20 | 52192737 | 52192741 | p1.(T)5 |
| 20 | 52192818 | 52192822 | p1.(C)5 |
| 20 | 52193078 | 52193082 | p1.(T)5 |
| 20 | 52193226 | 52193230 | p1.(C)5 |
| 20 | 52193248 | 52193252 | p1.(T)5 |
| 20 | 52193420 | 52193425 | p1.(T)6 |
| 20 | 52193453 | 52193457 | p1.(T)5 |
| 20 | 52193489 | 52193493 | p1.(T)5 |
| 20 | 52193554 | 52193558 | p1.(A)5 |
| 20 | 52193620 | 52193625 | p1.(A)6 |
| 20 | 52193636 | 52193640 | p1.(T)5 |
| 20 | 52193687 | 52193691 | p1.(T)5 |
| 20 | 52193728 | 52193732 | p1.(T)5 |
| 20 | 52193812 | 52193816 | p1.(T)5 |
| 20 | 52194917 | 52194921 | p1.(A)5 |
| 20 | 52194966 | 52194970 | p1.(T)5 |
| 20 | 52194983 | 52194987 | p1.(T)5 |
| 20 | 52194998 | 52195002 | p1.(A)5 |
| 20 | 52198642 | 52198648 | p1.(T)7 |
| 20 | 52198816 | 52198820 | p1.(C)5 |
| 20 | 52198848 | 52198852 | p1.(T)5 |
| 20 | 52199202 | 52199208 | p1.(T)7 |
| 20 | 54945199 | 54945203 | p1.(C)5 |
| 20 | 54945602 | 54945606 | p1.(A)5 |
| 20 | 54945677 | 54945681 | p1.(G)5 |
| 20 | 54959326 | 54959331 | p1.(T)6 |
| 20 | 54959341 | 54959345 | p1.(T)5 |
| 20 | 57478614 | 57478618 | p1.(A)5 |
| 20 | 57478757 | 57478762 | p1.(C)6 |
| 20 | 57484205 | 57484209 | p1.(T)5 |
| 20 | 57485378 | 57485382 | p1.(T)5 |
| 20 | 57485893 | 57485897 | p1.(C)5 |
| 20 | 60739192 | 60739196 | p1.(C)5 |
| 20 | 60740566 | 60740571 | p1.(G)6 |
| 20 | 62331943 | 62331947 | p1.(C)5 |
| 20 | 62332057 | 62332061 | p1.(G)5 |
| 21 | 34399229 | 34399233 | p1.(T)5 |
| 21 | 34399276 | 34399280 | p1.(G)5 |
| 21 | 34399702 | 34399706 | p1.(G)5 |
| 21 | 34399986 | 34399991 | p1.(G)6 |
| 21 | 34399993 | 34399997 | p1.(G)5 |
| 21 | 34400017 | 34400021 | p1.(G)5 |
| 21 | 36164486 | 36164490 | p1.(G)5 |
| 21 | 36164839 | 36164843 | p1.(G)5 |
| 21 | 36206777 | 36206781 | p1.(G)5 |
| 21 | 36421232 | 36421236 | p1.(G)5 |
| 21 | 39755389 | 39755393 | p1.(C)5 |
| 21 | 39755427 | 39755433 | p1.(A)7 |
| 21 | 39755554 | 39755559 | p1.(G)6 |
| 21 | 39762972 | 39762976 | p1.(A)5 |
| 21 | 39764347 | 39764352 | p1.(G)6 |
| 21 | 39774562 | 39774576 | p5.(ACAAA)3 |
| 21 | 39775416 | 39775420 | p1.(G)5 |
| 21 | 39775477 | 39775481 | p1.(G)5 |
| 21 | 39795373 | 39795377 | p1.(G)5 |
| 21 | 39817381 | 39817385 | p1.(G)5 |
| 21 | 42842590 | 42842595 | p1.(C)6 |
| 21 | 42843818 | 42843822 | p1.(T)5 |
| 21 | 42845287 | 42845291 | p1.(G)5 |
| 21 | 42845355 | 42845359 | p1.(C)5 |
| 21 | 42848552 | 42848556 | p1.(A)5 |
| 21 | 42851110 | 42851115 | p1.(T)6 |
| 21 | 42851199 | 42851203 | p1.(A)5 |
| 21 | 42851212 | 42851217 | p1.(A)6 |
| 21 | 42866377 | 42866381 | p1.(G)5 |
| 21 | 44513126 | 44513139 | p1.(T)6(A)8 |
| 21 | 44513198 | 44513202 | p1.(A)5 |
| 21 | 44515638 | 44515642 | p1.(A)5 |
| 21 | 44515807 | 44515812 | p1.(A)6 |
| 21 | 44527612 | 44527623 | p3.(CCG)4 |
| 22 | 19167754 | 19167760 | p1.(A)7 |
| 22 | 19195739 | 19195743 | p1.(A)5 |
| 22 | 19195776 | 19195780 | p1.(T)5 |
| 22 | 19203775 | 19203779 | p1.(A)5 |
| 22 | 19208924 | 19208928 | p1.(C)5 |
| 22 | 19208953 | 19208957 | p1.(G)5 |
| 22 | 19209481 | 19209486 | p1.(T)6 |
| 22 | 19220690 | 19220694 | p1.(T)5 |
| 22 | 19223226 | 19223230 | p1.(T)5 |
| 22 | 19226837 | 19226842 | p1.(A)6 |
| 22 | 19263362 | 19263368 | p1.(A)7 |
| 22 | 19279181 | 19279195 | p3.(CGG)5 |
| 22 | 19707424 | 19707428 | p1.(C)5 |
| 22 | 19707962 | 19707966 | p1.(C)5 |
| 22 | 19709156 | 19709160 | p1.(C)5 |
| 22 | 19709245 | 19709249 | p1.(G)5 |
| 22 | 21288188 | 21288192 | p1.(A)5 |
| 22 | 23523205 | 23523209 | p1.(C)5 |
| 22 | 23523395 | 23523399 | p1.(C)5 |
| 22 | 23523564 | 23523568 | p1.(G)5 |
| 22 | 23523586 | 23523591 | p1.(C)6 |
| 22 | 23523784 | 23523788 | p1.(A)5 |
| 22 | 23523816 | 23523820 | p1.(G)5 |
| 22 | 23523832 | 23523836 | p1.(C)5 |
| 22 | 23523903 | 23523907 | p1.(C)5 |
| 22 | 23523952 | 23523956 | p1.(C)5 |
| 22 | 23524000 | 23524004 | p1.(G)5 |
| 22 | 23524097 | 23524102 | p1.(C)6 |
| 22 | 23524294 | 23524299 | p1.(C)6 |
| 22 | 23603670 | 23603674 | p1.(C)5 |
| 22 | 23610586 | 23610590 | p1.(C)5 |
| 22 | 23626208 | 23626212 | p1.(G)5 |
| 22 | 23627310 | 23627314 | p1.(C)5 |
| 22 | 23631814 | 23631820 | p1.(C)7 |
| 22 | 24129357 | 24129368 | p3.(ATG)4 |
| 22 | 24134057 | 24134063 | p1.(A)7 |
| 22 | 24135859 | 24135864 | p1.(C)6 |
| 22 | 24143260 | 24143264 | p1.(C)5 |
| 22 | 28193075 | 28193079 | p1.(G)5 |
| 22 | 28193164 | 28193168 | p1.(C)5 |
| 22 | 28193233 | 28193237 | p1.(G)5 |
| 22 | 28193333 | 28193337 | p1.(G)5 |
| 22 | 28193474 | 28193478 | p1.(C)5 |
| 22 | 28193492 | 28193496 | p1.(C)5 |
| 22 | 28193499 | 28193503 | p1.(C)5 |
| 22 | 28193569 | 28193573 | p1.(C)5 |
| 22 | 28193623 | 28193627 | p1.(C)5 |
| 22 | 28193690 | 28193694 | p1.(T)5 |
| 22 | 28193719 | 28193723 | p1.(C)5 |
| 22 | 28193772 | 28193776 | p1.(G)5 |
| 22 | 28193792 | 28193796 | p1.(C)5 |
| 22 | 28194006 | 28194010 | p1.(G)5 |
| 22 | 28194184 | 28194188 | p1.(C)5 |
| 22 | 28194217 | 28194221 | p1.(C)5 |
| 22 | 28194231 | 28194236 | p1.(G)6 |
| 22 | 28194307 | 28194311 | p1.(C)5 |
| 22 | 28194340 | 28194344 | p1.(G)5 |
| 22 | 28194421 | 28194425 | p1.(C)5 |
| 22 | 28194512 | 28194516 | p1.(C)5 |
| 22 | 28194535 | 28194539 | p1.(G)5 |
| 22 | 28194881 | 28194898 | p3.(GCT)6 |
| 22 | 28194913 | 28194930 | p3.(TGC)6 |
| 22 | 28194934 | 28194960 | p3.(TGC)9 |
| 22 | 28195185 | 28195190 | p1.(G)6 |
| 22 | 28195625 | 28195639 | p3.(GCT)5 |
| 22 | 28195729 | 28195733 | p1.(C)5 |
| 22 | 28196140 | 28196144 | p1.(C)5 |
| 22 | 28196185 | 28196189 | p1.(C)5 |
| 22 | 28196195 | 28196199 | p1.(G)5 |
| 22 | 28196286 | 28196291 | p1.(C)6 |
| 22 | 28196356 | 28196360 | p1.(G)5 |
| 22 | 28196398 | 28196408 | p1.(G)5(C)6 |
| 22 | 28196539 | 28196544 | p1.(G)6 |
| 22 | 28196549 | 28196553 | p1.(G)5 |
| 22 | 29091793 | 29091797 | p1.(G)5 |
| 22 | 29095849 | 29095853 | p1.(A)5 |
| 22 | 29095914 | 29095918 | p1.(C)5 |
| 22 | 29108014 | 29108018 | p1.(A)5 |
| 22 | 29121096 | 29121100 | p1.(T)5 |
| 22 | 29130434 | 29130438 | p1.(G)5 |
| 22 | 29130719 | 29130723 | p1.(A)5 |
| 22 | 29669714 | 29669722 | p1.(T)9 |
| 22 | 29678431 | 29678435 | p1.(G)5 |
| 22 | 29684714 | 29684718 | p1.(G)5 |
| 22 | 29693944 | 29693948 | p1.(T)5 |
| 22 | 29694746 | 29694750 | p1.(G)5 |
| 22 | 29695315 | 29695319 | p1.(C)5 |
| 22 | 29695765 | 29695775 | p1.(G)6(C)5 |
| 22 | 29695830 | 29695834 | p1.(A)5 |
| 22 | 29999923 | 29999928 | p1.(G)6 |
| 22 | 30050682 | 30050686 | p1.(T)5 |
| 22 | 30050708 | 30050712 | p1.(A)5 |
| 22 | 30051572 | 30051577 | p1.(T)6 |
| 22 | 30057197 | 30057201 | p1.(A)5 |
| 22 | 30057270 | 30057274 | p1.(C)5 |
| 22 | 30070939 | 30070943 | p1.(G)5 |
| 22 | 30074321 | 30074325 | p1.(C)5 |
| 22 | 30077585 | 30077589 | p1.(A)5 |
| 22 | 31737529 | 31737535 | p1.(T)7 |
| 22 | 31737674 | 31737678 | p1.(A)5 |
| 22 | 31740553 | 31740557 | p1.(G)5 |
| 22 | 31740735 | 31740739 | p1.(C)5 |
| 22 | 31740856 | 31740861 | p1.(G)6 |
| 22 | 31741056 | 31741061 | p1.(G)6 |
| 22 | 31741089 | 31741093 | p1.(G)5 |
| 22 | 31741290 | 31741294 | p1.(C)5 |
| 22 | 31741323 | 31741327 | p1.(C)5 |
| 22 | 31741341 | 31741345 | p1.(C)5 |
| 22 | 31741482 | 31741486 | p1.(T)5 |
| 22 | 31741597 | 31741601 | p1.(C)5 |
| 22 | 36678673 | 36678684 | p4.(TGTC)3 |
| 22 | 36678834 | 36678838 | p1.(G)5 |
| 22 | 36684462 | 36684466 | p1.(G)5 |
| 22 | 36688264 | 36688268 | p1.(T)5 |
| 22 | 36689419 | 36689433 | p3.(CCT)5 |
| 22 | 36690293 | 36690298 | p1.(C)6 |
| 22 | 36694956 | 36694960 | p1.(C)5 |
| 22 | 36696225 | 36696229 | p1.(T)5 |
| 22 | 36696281 | 36696292 | p3.(CTC)4 |
| 22 | 36696316 | 36696320 | p1.(G)5 |
| 22 | 36696914 | 36696925 | p3.(TCT)4 |
| 22 | 36696948 | 36696962 | p3.(CTC)5 |
| 22 | 36708257 | 36708262 | p1.(G)6 |
| 22 | 36714377 | 36714381 | p1.(T)5 |
| 22 | 36737462 | 36737466 | p1.(G)5 |
| 22 | 36745146 | 36745157 | p4.(GGCT)3 |
| 22 | 36745238 | 36745242 | p1.(T)5 |
| 22 | 38369476 | 38369481 | p1.(G)6 |
| 22 | 38369496 | 38369500 | p1.(C)5 |
| 22 | 38369708 | 38369712 | p1.(G)5 |
| 22 | 38369803 | 38369807 | p1.(G)5 |
| 22 | 38370110 | 38370114 | p1.(C)5 |
| 22 | 38373905 | 38373909 | p1.(G)5 |
| 22 | 38379676 | 38379690 | p3.(CCG)5 |
| 22 | 38379796 | 38379800 | p1.(C)5 |
| 22 | 39621797 | 39621802 | p1.(G)6 |
| 22 | 39626106 | 39626111 | p1.(C)6 |
| 22 | 39631876 | 39631880 | p1.(C)5 |
| 22 | 39640023 | 39640027 | p1.(C)5 |
| 22 | 40807460 | 40807464 | p1.(G)5 |
| 22 | 40807477 | 40807482 | p1.(G)6 |
| 22 | 40807617 | 40807622 | p1.(G)6 |
| 22 | 40807757 | 40807768 | p4.(AGGG)3 |
| 22 | 40807795 | 40807799 | p1.(G)5 |
| 22 | 40807831 | 40807836 | p1.(G)6 |
| 22 | 40814383 | 40814387 | p1.(G)5 |
| 22 | 40814485 | 40814489 | p1.(C)5 |
| 22 | 40814527 | 40814532 | p1.(G)6 |
| 22 | 40814586 | 40814590 | p1.(G)5 |
| 22 | 40814720 | 40814724 | p1.(G)5 |
| 22 | 40814731 | 40814736 | p1.(G)6 |
| 22 | 40814738 | 40814742 | p1.(G)5 |
| 22 | 40814744 | 40814748 | p1.(G)5 |
| 22 | 40814900 | 40814905 | p1.(C)6 |
| 22 | 40814932 | 40814936 | p1.(G)5 |
| 22 | 40815026 | 40815030 | p1.(G)5 |
| 22 | 40815071 | 40815075 | p1.(G)5 |
| 22 | 40815086 | 40815091 | p1.(G)6 |
| 22 | 40815269 | 40815273 | p1.(G)5 |
| 22 | 40816542 | 40816548 | p1.(G)7 |
| 22 | 40816887 | 40816901 | p3.(TGC)5 |
| 22 | 40816930 | 40816941 | p3.(GCT)4 |
| 22 | 40816970 | 40816975 | p1.(G)6 |
| 22 | 40816978 | 40816983 | p1.(C)6 |
| 22 | 40817001 | 40817006 | p1.(G)6 |
| 22 | 40819552 | 40819556 | p1.(G)5 |
| 22 | 40819613 | 40819617 | p1.(G)5 |
| 22 | 40820216 | 40820220 | p1.(G)5 |
| 22 | 40820312 | 40820316 | p1.(G)5 |
| 22 | 40827491 | 40827496 | p1.(A)6 |
| 22 | 41489005 | 41489009 | p1.(A)5 |
| 22 | 41513810 | 41513814 | p1.(C)5 |
| 22 | 41521966 | 41521970 | p1.(A)5 |
| 22 | 41522009 | 41522013 | p1.(A)5 |
| 22 | 41525970 | 41525974 | p1.(C)5 |
| 22 | 41525977 | 41525981 | p1.(A)5 |
| 22 | 41527458 | 41527462 | p1.(C)5 |
| 22 | 41533648 | 41533652 | p1.(T)5 |
| 22 | 41536135 | 41536139 | p1.(T)5 |
| 22 | 41542730 | 41542735 | p1.(T)6 |
| 22 | 41543876 | 41543880 | p1.(C)5 |
| 22 | 41545025 | 41545038 | p1.(T)14 |
| 22 | 41545901 | 41545905 | p1.(C)5 |
| 22 | 41545921 | 41545925 | p1.(C)5 |
| 22 | 41546023 | 41546027 | p1.(C)5 |
| 22 | 41547821 | 41547828 | p1.(T)8 |
| 22 | 41548269 | 41548273 | p1.(A)5 |
| 22 | 41548348 | 41548352 | p1.(A)5 |
| 22 | 41550985 | 41550995 | p1.(T)11 |
| 22 | 41556700 | 41556704 | p1.(G)5 |
| 22 | 41566525 | 41566531 | p1.(A)7 |
| 22 | 41568507 | 41568511 | p1.(T)5 |
| 22 | 41569645 | 41569649 | p1.(A)5 |
| 22 | 41569654 | 41569658 | p1.(A)5 |
| 22 | 41569681 | 41569685 | p1.(A)5 |
| 22 | 41572243 | 41572247 | p1.(T)5 |
| 22 | 41572765 | 41572770 | p1.(T)6 |
| 22 | 41572810 | 41572814 | p1.(A)5 |
| 22 | 41573261 | 41573265 | p1.(C)5 |
| 22 | 41573476 | 41573480 | p1.(C)5 |
| 22 | 41573555 | 41573559 | p1.(T)5 |
| 22 | 41573584 | 41573588 | p1.(C)5 |
| 22 | 41573603 | 41573607 | p1.(C)5 |
| 22 | 41573946 | 41573950 | p1.(C)5 |
| 22 | 41574081 | 41574085 | p1.(G)5 |
| 22 | 41574201 | 41574205 | p1.(C)5 |
| 22 | 41574379 | 41574390 | p3.(CAG)4 |
| 22 | 41574552 | 41574556 | p1.(C)5 |
| 22 | 41574679 | 41574685 | p1.(C)7 |
| 22 | 41574986 | 41574992 | p1.(A)7 |
| 22 | 42526682 | 42526686 | p1.(G)5 |

Patient DNA was sequenced by NGS using the 592-gene panel. See Tables 7-10. We examined the 7,317 target microsatellite loci and compared them to the reference genome hg19 from the UCSC Genome Browser database (hgdownload.cse.ucsc.edu/goldenPath/hg19/bigZips/). The number of microsatellite loci that were altered by somatic insertion or deletion was counted for each patient sample. Only insertions or deletions that increased or decreased the number of repeats were considered. A locus was not counted more than once even if it had multiple lengths of insertions or deletions. Thresholds were calibrated based on comparison of total number of altered loci per patient to MSI-FA results with the aim to maximize sensitivity while maintaining an appropriately high specificity, positive predictive value (PPV), and negative predictive value (NPV).

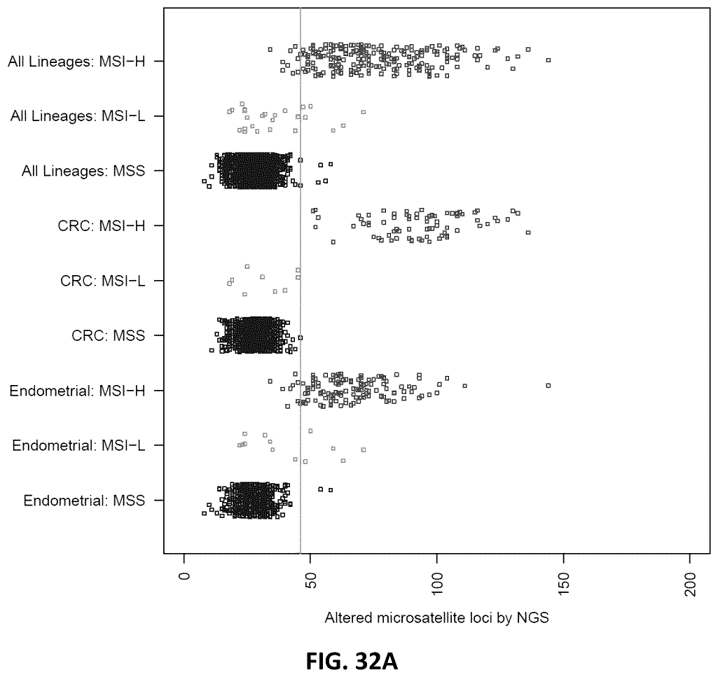

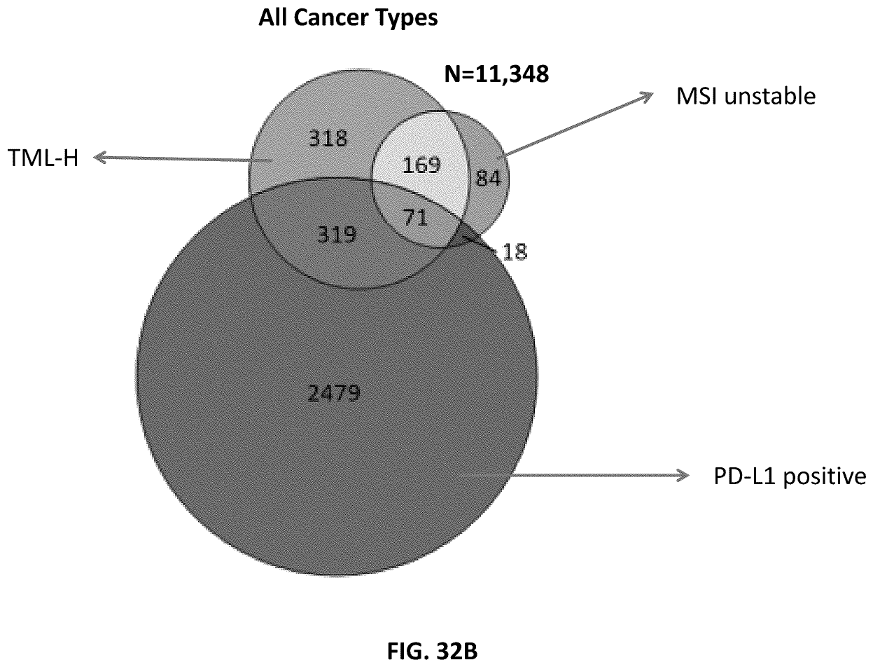

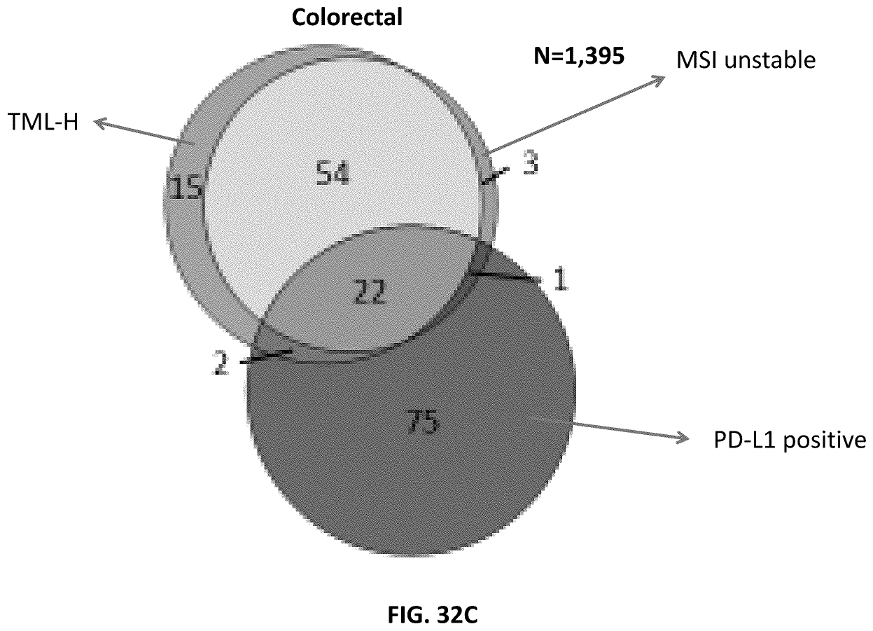

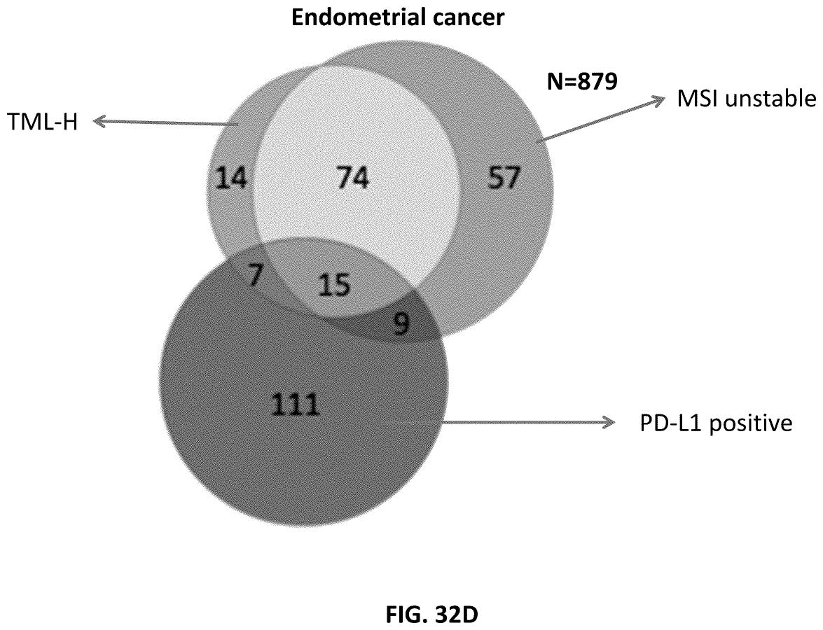

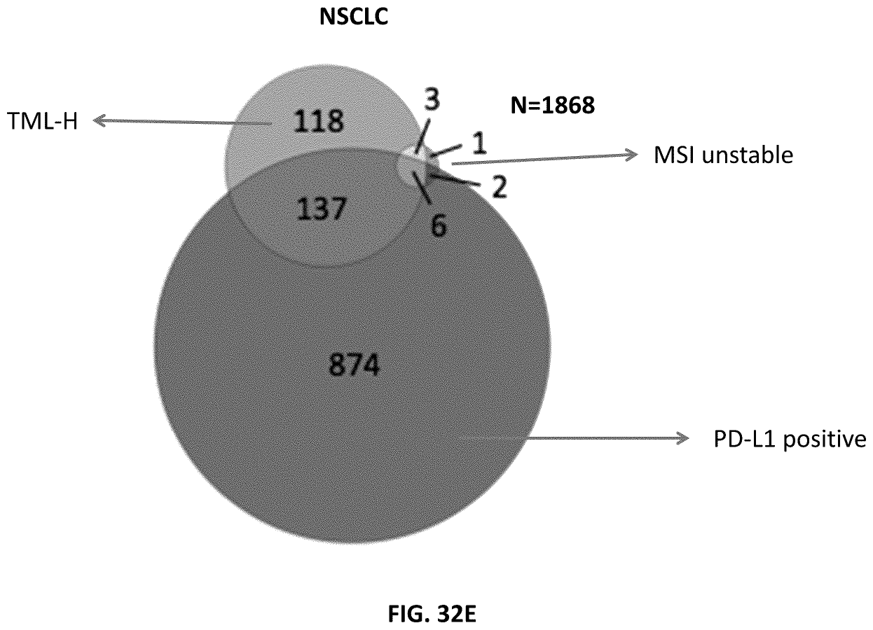

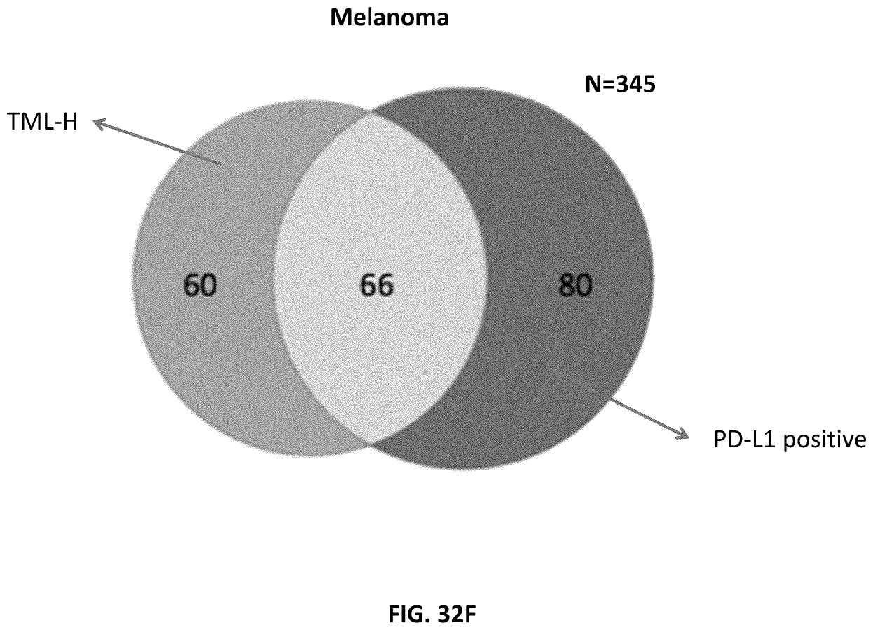

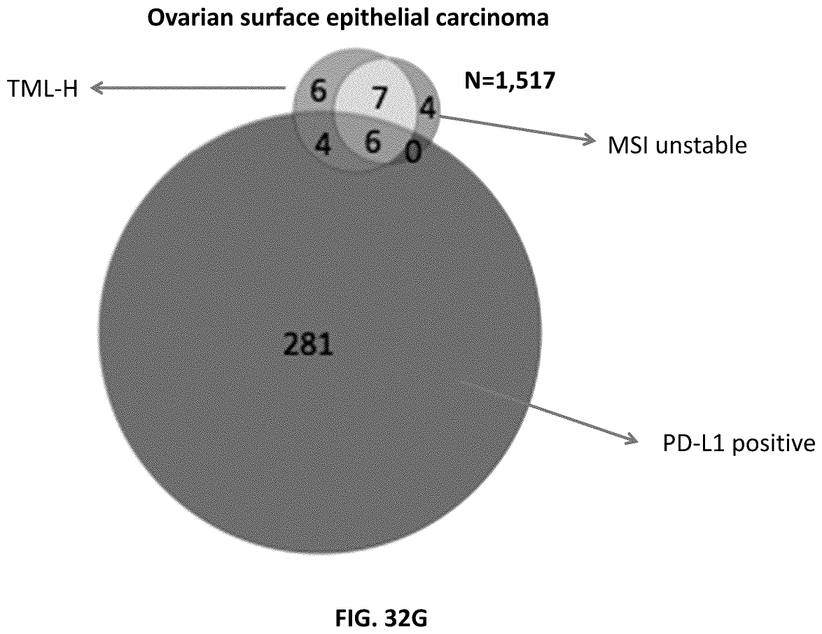

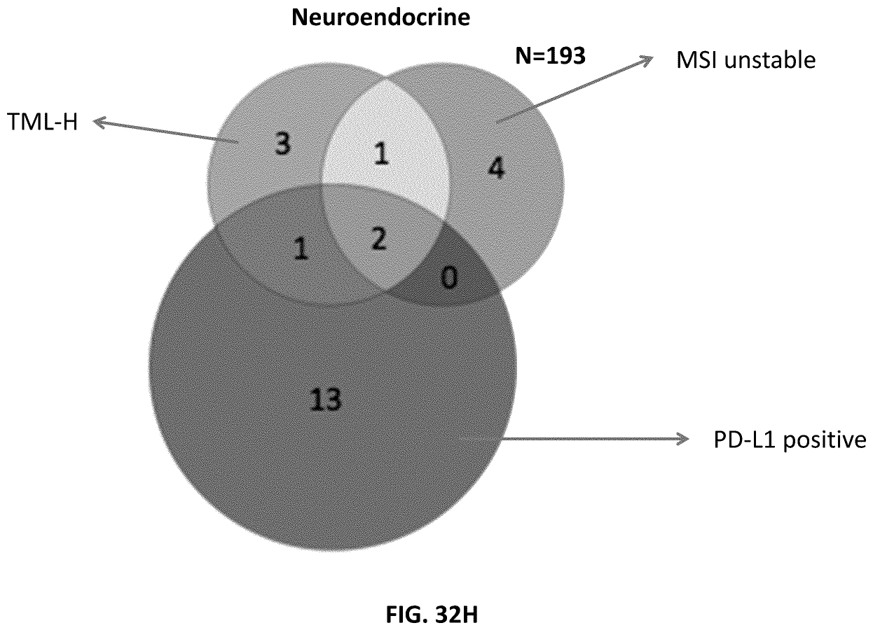

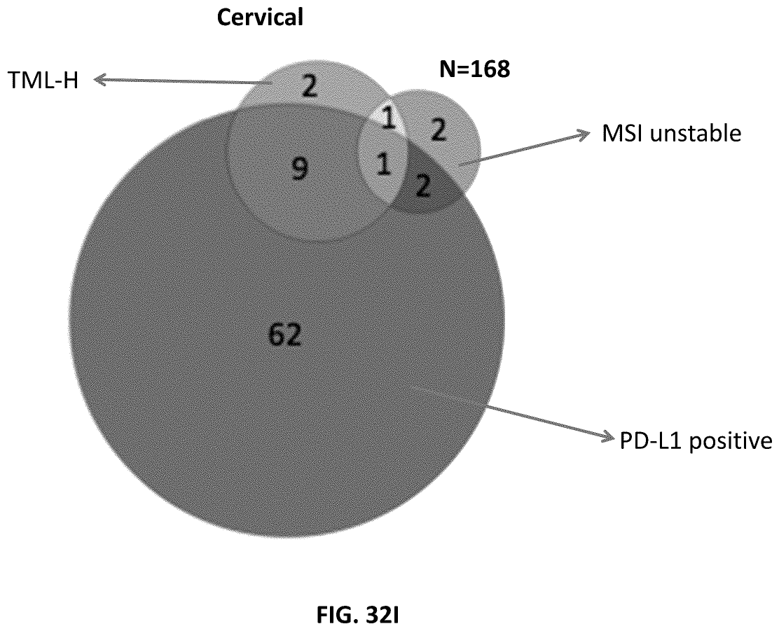

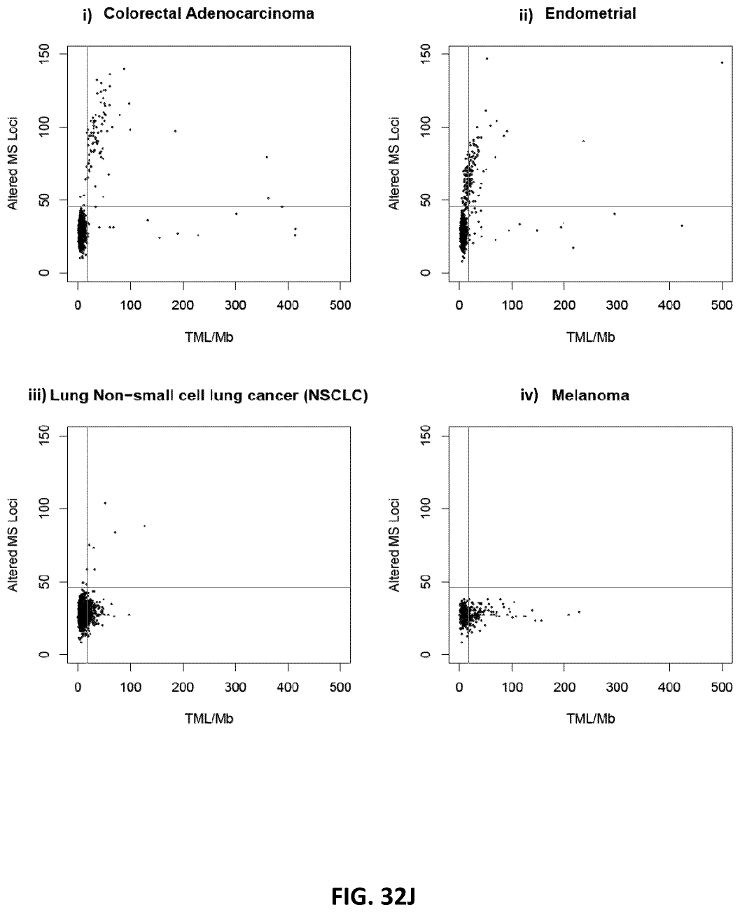

We calibrated our thresholds by comparing the total altered loci per patient by NGS to the PCR-based MSI FA results from 2,189 cases that included 26 distinct cancer lineages, consisting mostly of colorectal (n=1,193) and endometrial (n=709) cases. See FIG. 32A. The figure shows analysis by PCR FA (y-axis) classified cases as MSS, MSI-low (MSI-L), or MSI-high (MSI-H), and NGS (x-axis) classified cases as MSS (<46 altered microsatellite loci/Mb) or MSI-H (≥46 altered microsatellite loci/Mb). Cases include all cancer lineages (n=2,189), colorectal adenocarcinoma (CRC; n=1,193), and endometrial cancer (n=708). Abbreviations in FIG. 32A: Mb, megabase; MSI-H, microsatellite high; MSI-L, microsatellite low; MSS, microsatellite stable.

An appropriate threshold aims to provide acceptably high levels of sensitivity, specificity, and positive and negative predictive values across cancer types, while capturing most if not all MSI-H by FA cases of colorectal cancer. Based on this analysis, samples having 46 or more loci with insertions or deletions were considered MSI-H.

### Total Mutation Burden

TMB was calculated based on the number of nonsynonymous somatic mutations identified by NGS, while excluding any known single nucleotide polymorphisms (SNPs) in dbSNP (version 137) or in the 1000 Genomes Project database (phase 3; www.intemationalgenome.org/). [20] TMB is reported as mutations per Mb sequenced. The threshold for determining high TMB as greater than or equal to 17 mutations/megabase was established by comparing TMB with MSI by FA in CRC cases, based on reports of TMB having high concordance with MSI in CRC. [7,21]

### PD-L1 IHC

IHC analysis was performed on slides of FFPE tumor samples using automated staining techniques. The procedures met the standards and requirements of the College of American Pathologists.

The primary antibody against PD-L1 was SP142 (Spring Bioscience, Pleasanton, CA), except for NSCLC tumors tested after January 2016. For NSCLC tumors tested after January 2016, the primary PD-L1 antibody clone was 22c3 (Dako, Santa Clara, CA). For the calculations in this Example, staining for both antibodies was considered positive if there was staining on >1% of tumor cells.

### Mismatch Repair Protein IHC

MMR protein expression was tested by IHC using antibody clones (MLH1, M1 antibody; MSH2, G2191129 antibody; MSH6, 44 antibody; PMS2, EPR3947 antibody (Ventana Medical Systems, Inc., Tucson, AZ)). The complete absence of protein expression (0+in 100% of cells) was considered a loss of MMR, and thus dMMR.

### Cancer Types Analyzed by PCR-FA and by MSI-NGS

Matched cases analyzed both by PCR-FA and by MSI-NGS included the following cancer types: bladder cancer (n=3), breast carcinoma (n=16), cervical cancer (n=2), cholangiocarcinoma (n=17), colorectal adenocarcinoma (n=1193), endometrial cancer (n=708), esophageal and esophagogastric junction carcinoma (n=7), extrahepatic bile duct adenocarcinoma (n=2), gastric adenocarcinoma (n=10), gastrointestinal stromal tumors (n=2), glioblastoma (n=9), liver hepatocellular carcinoma (n=8), lymphoma (n=2), malignant solitary fibrous tumor of the pleura (n=1), melanoma (n=4), neuroendocrine tumors (n=10), none of these (n=21), NSCLC (n=5), other female genital tract malignancy (n=12), ovarian surface epithelial carcinomas (n=15), pancreatic adenocarcinoma (n=44), prostatic adenocarcinoma (n=1), small intestinal malignancies (n=7), soft tissue tumors (n=1), thyroid carcinoma (n=1), uterine sarcoma (n=87), and uveal melanoma (n=1).

### Cancer Types Analyzed by IHC and by MSI-NGS

Matched cases analyzed both by IHC and by MSI-NGS included the following cancer types: bladder cancer (n=4), breast carcinoma (n=18), cervical cancer (n=1), cholangiocarcinoma (n=21), colorectal adenocarcinoma (n=925), endometrial cancer (n=445), esophageal and esophagogastric junction carcinoma (n=8), gastric adenocarcinoma (n=15), gastrointestinal stromal tumors (n=3), glioblastoma (n=53), head and neck squamous cell carcinoma (n=1), kidney cancer (n=1), liver hepatocellular carcinoma (n=12), low-grade glioma (n=7), lymphoma (n=3), melanoma (n=2), neuroendocrine tumors (n=10), none of these (n=38), NSCLC (n=6), other female genital tract malignancy (n=3), ovarian surface epithelial carcinomas (n=17), pancreatic adenocarcinoma (n=318), prostatic adenocarcinoma (n=2), small intestinal malignancies (n=5), soft tissue tumors (n=1), uterine sarcoma (n=65), and uveal melanoma (n=2).

### Results

Matched MSI FA PCR and 592-gene NGS assays from 2,189 cases (FIG. 32A and Table 17) were used to calibrate the MSI NGS assay to classify samples as MSI-H or microsatellite stable (MSS). A cutoff of >46 altered loci was chosen with with goal of optimizing the performance of the MSI-NGS test in CRC and endometrial cancers, which are cancer types for which MSI testing has traditionally had the highest clinical relevance. See FIG. 32A. Performance was maintained when this cutoff was used across all 2,189 FA-matched cases that spanned 26 cancer types (sensitivity 95.8% [95% confidence interval (CI) 92.24, 98.08], specificity 99.4% [95% CI 98.94, 99.69], positive predictive value (PPV) 94.5% [95% CI 90.62, 97.14], and negative predictive value (NPV) 99.2% [95% CI, 98.75, 99.57]). For purposes of calculating the MSI NGS performance metrics, cases categorized as MSI-Low by FA were grouped with the MSS FA cohort. Since patients with MSI-L tumors are most often treated in a manner similar to patients with MSS tumors in the clinic, grouping MSI-L with MSS is reasonable.

TABLE 17
Classification of MSI by NGS compared with PCR fragment analysis for 2,189 matched cases

| | | | Next-Generation Sequencing | | | | | |
|---|---|---|---|---|---|---|---|---|
| | | | MSI-H | MSS | Sensitivity % | Specificity % | PPV % (95% | NPV % (95% |
| | | | No. of Patients | | (95% CI) | (95% CI) | CI) | CI) |
| All types of | Fragment | MSS | 6 | 1941 | 95.8 | 99.4 | 94.5 | 99.2 |
| cancer (FA data | Analysis | MSI-L | 6 | 20 | (92.24, 98.08) | (98.94, 99.69) | (90.62, | (98.75, 99.57) |
| for n = 2,189) | | | | | | | 97.14) | |
| | | MSI-H | 207 | 9 | | | | |
| Colorectal cancer | | MSS | 1 | 1108 | 100.0 | 99.9 | 98.7 | 99.6 |
| (FA data for n = | | MSI-L | 0 | 9 | (95.2, 100) | (99.5, 100) | (92.89, | (99.09, 99.9) |
| 1,193) | | | | | | | 99.97) | |
| | | MSI-H | 75 | 0 | | | | |
| Non-colorectal | | MSS | 5 | 833 | 93.6 | 98.7 | 92.3 | 98.7 |
| cancer (FA data | | MSI-L | 6 | 11 | (88.23, 97.04) | (97.71, 99.36) | (86.65, 96.1) | (97.71, 99.36) |
| for n = 996) | | MSI-H | 132 | 9 | | | | |
| Endometrial | | MSS | 2 | 562 | 93.9 | 98.8 | 94.6 | 98.4 |
| cancer (FA data | | MSI-L | 5 | 8 | (88.32, 97.33) | (97.52, 99.51) | (89.22, | (97.07, 99.29) |
| for n = 709) | | | | | | | 97.81) | |
| | | MSI-H | 123 | 8 | | | | |

Abbreviations in Table 17: CRC, colorectal cancer; FA, fragment analysis; MMR, mismatch repair; MSI-L, microsatellite instability-low; MSI-H, microsatellite instability-high; MSS, microsatellite stable; NGS, next generation sequencing; NPV, negative predictive value; PPV, positive predictive value.

An additional comparison involved 1,986 cases that were examined both by MSI-NGS and by IHC for MMR protein status. See Table 18. Cases with dMMR protein status were identified by IHC in 171 cases (8.6%), while NGS identified 156 cases (7.9%). Compared with IHC for MMR proteins, across 26 cancer types, NGS had a sensitivity of 87.1%, specificity of 99.6%, PPV of 95.5%, and NPV of 98.8%. Compared with IHC for MMR proteins, NGS of CRC cases had a sensitivity of 91.7%, specificity of 99.7%, PPV of 94.8%, and NPV of 99.4%.

TABLE 18
Classification of microsatellite instability by NGS compared with MMR by IHC

| | | | Next-generation sequencing | | | | | |
|---|---|---|---|---|---|---|---|---|
| | | | MSI-H | MSS | Sensitivity | Specificity | | |
| | | | No. of Patients | | (%) | (%) | PPV (%) | NPV (%) |
| All types of | IHC MMR | dMMR | 149 | 22 | 87.1 | 99.6 | 95.5 | 98.8 |
| cancer | | MMR-P | 7 | 1808 | | | | |
| (n = 1,986) | | | | | | | | |
| CRC (n = 925) | | dMMR | 55 | 5 | 91.7 | 99.7 | 94.8 | 99.4 |
| | | MMR-P | 3 | 862 | | | | |
| Non-CRC | | dMMR | 94 | 17 | 84.7 | 99.6 | 95.9 | 98.2 |
| (n = 1061) | | MMR-P | 4 | 946 | | | | |

Abbreviations in Table 17: IHC, immunohistochemistry; MMR, mismatch repair; dMMR, deficient mismatch repair; MMR-P, mismatch repair proficient; MSI-H, microsatellite instability-high; MSS, microsatellite stable; NPV, negative predictive value; PPV, positive predictive value.

The highest percentage of MSI-H cases were endometrial cancer (18%), followed by gastric adenocarcinoma (9%), small intestinal malignancies (8%), and colorectal adenocarcinoma (6%). Cancer types with no MSI-H included melanoma (0 of 360 cases), bladder cancer (0 of 144), head and neck squamous carcinoma (0 of 118), low-grade glioma 90 of 107), gastrointestinal stromal cancers (0 of 65), and thymic cancer (0 of 28).

The relationship between TMB, MSI, and PD-L1 was explored by analyzing 11,348 cases that had results for all three assays. See FIG. 32B and Table 19. In this set, the overall rate of MSI-H was 3.0%. Overall high TMB was 7.7% and PD-L1 positivity was 25.4%. Among MSI-H cases, 70% were also high TMB (62.6% with TMB removed). Among high TMB cases, 27% were also MSI-H. Only 0.6% of the cases were positive for all three markers, whereas 69.5% of the cases were negative for all three. Of the total cohort, 26% of TMS-H cases were PD-L1 positive whereas 44% of TMB high cases were PD-L1 positive.

The overlap between the biomarkers TMB, MSJ, and PD-L1 differed among cancer types. See FIGS. 32C-321 (FIG. 32C shows colorectal cancer (CRC); FIG. 32D shows endometrial cancer; FIG. 32E shows non-small cell lung cancer (NSCLC); FIG. 32F shows melanoma; FIG. 32G shows ovarian surface epithelial carcinoma; FIG. 32H shows neuroendocrine cancer; FIG. 321 shows cervical cancer) and Table 19. High TMB and MSJ-H had 95% overlap for CRC, which was expected, since the TMB cutoff was based on CRC MSJ-FA results. However, 57% of MSJ-H endometrial cancer cases were also high TMB. Likewise, ovarian, neuroendocrine, and cervical cancers also had significant percentages of MSJ-H cases that were not TMB high. In contrast, NSCLC and melanoma had few or no MSJ-H cases, while still having a significant number of high TMB cases.

TABLE 19
Biomarkers by NGS across cancer types

| | | | | | | | | | | | | | | MSI + | | | |
|---|---|---|---|---|---|---|---|---|---|---|---|---|---|---|---|---|---|
| | | | | | | | | | | MSI + | | | | TMB + | | None of | |
| | | | | | | | | MSI + | | PD- | | TMB + | | PD- | | these | |
| | | MSI | | TMB | | PD-L1 | | TMB | | L1 | | PD-L1 | | L1 | | biomarkers | |
| | N | n | % | n | % | n | % | n | % | n | % | n | % | n | % | n | % |
| All cancr types | 11348 | 342 | 3.0 | 877 | 7.7 | 2887 | 25.5 | 240 | 2.1 | 89 | 0.8 | 390 | 3.4 | 71 | 0.6 | 7890 | 69.5 |
| NSCLC | 1868 | 12 | 0.6 | 264 | 14.1 | 1013 | 54.3 | 9 | 0.5 | 8 | 0.4 | 143 | 7.7 | 6 | 0.3 | 733 | 39.2 |
| Ovarian surface | 1517 | 17 | 1.1 | 24 | 1.6 | 291 | 19.2 | 13 | 0.9 | 6 | 0.4 | 10 | 0.7 | 6 | 0.4 | 1208 | 79.6 |
| epithelial | | | | | | | | | | | | | | | | | |
| carcinomas | | | | | | | | | | | | | | | | | |
| Colorectal | 1395 | 80 | 5.7 | 93 | 6.7 | 100 | 7.2 | 76 | 5.4 | 23 | 1.6 | 24 | 1.7 | 22 | 1.6 | 1223 | 87.7 |
| adenocarcinoma | | | | | | | | | | | | | | | | | |
| Breast carcinoma | 1024 | 6 | 0.6 | 31 | 3.0 | 99 | 9.7 | 4 | 0.4 | 1 | 0.1 | 4 | 0.4 | 1 | 0.1 | 896 | 87.5 |
| Endometrial | 879 | 155 | 17.6 | 110 | 12.5 | 142 | 16.2 | 89 | 10.1 | 24 | 2.7 | 22 | 2.5 | 15 | 1.7 | 592 | 67.3 |
| carcinoma | | | | | | | | | | | | | | | | | |
| None of these apply | 705 | 7 | 1.0 | 91 | 12.9 | 219 | 31.1 | 4 | 0.6 | 2 | 0.3 | 54 | 7.7 | 1 | 0.1 | 447 | 63.4 |
| Pancreatic | 518 | 6 | 1.2 | 6 | 1.2 | 112 | 21.6 | 4 | 0.8 | 3 | 0.6 | 2 | 0.4 | 2 | 0.4 | 401 | 77.4 |
| adenocarcinoma | | | | | | | | | | | | | | | | | |
| Glioblastoma | 427 | 3 | 0.7 | 15 | 3.5 | 106 | 24.8 | 3 | 0.7 | 1 | 0.2 | 5 | 1.2 | 1 | 0.2 | 311 | 72.8 |
| Melanoma | 345 | 0 | 0.0 | 126 | 36.5 | 146 | 42.3 | 0 | 0.0 | 0 | 0.0 | 66 | 19.1 | 0 | 0.0 | 139 | 40.3 |
| Soft tissue tumors | 283 | 1 | 0.4 | 11 | 3.9 | 59 | 20.8 | 0 | 0.0 | 0 | 0.0 | 7 | 2.5 | 0 | 0.0 | 219 | 77.4 |
| Neuroendocrine | 193 | 7 | 3.6 | 7 | 3.6 | 16 | 8.3 | 3 | 1.6 | 2 | 1.0 | 3 | 1.6 | 2 | 1.0 | 169 | 87.6 |
| tumors | | | | | | | | | | | | | | | | | |
| Prostatic | 191 | 4 | 2.1 | 5 | 2.6 | 13 | 6.8 | 4 | 2.1 | 1 | 0.5 | 1 | 0.5 | 1 | 0.5 | 174 | 91.1 |
| adenocarcinoma | | | | | | | | | | | | | | | | | |
| Esophageal and | 189 | 0 | 0.0 | 1 | 0.5 | 47 | 24.9 | 0 | 0.0 | 0 | 0.0 | 1 | 0.5 | 0 | 0.0 | 142 | 75.1 |
| esophagogastric | | | | | | | | | | | | | | | | | |
| junction carcinoma | | | | | | | | | | | | | | | | | |
| Gastric | 184 | 16 | 8.7 | 16 | 8.7 | 34 | 18.5 | 15 | 8.2 | 8 | 4.3 | 9 | 4.9 | 8 | 4.3 | 142 | 77.2 |
| adenocarcinoma | | | | | | | | | | | | | | | | | |
| Cholangiocarcinoma | 177 | 4 | 2.3 | 6 | 3.4 | 33 | 18.6 | 3 | 1.7 | 1 | 0.6 | 1 | 0.6 | 0 | 0.0 | 139 | 78.5 |
| Cervical cancer | 168 | 6 | 3.6 | 13 | 7.7 | 74 | 44.0 | 2 | 1.2 | 3 | 1.8 | 10 | 6.0 | 1 | 0.6 | 89 | 53.0 |
| Kidney cancer | 155 | 1 | 0.6 | 1 | 0.6 | 46 | 29.7 | 0 | 0.0 | 1 | 0.6 | 0 | 0.0 | 0 | 0.0 | 108 | 69.7 |
| Bladder cancer | 143 | 0 | 0.0 | 24 | 16.8 | 61 | 42.7 | 0 | 0.0 | 0 | 0.0 | 9 | 6.3 | 0 | 0.0 | 67 | 46.9 |
| Uterine sarcoma | 128 | 3 | 2.3 | 3 | 2.3 | 24 | 18.8 | 1 | 0.8 | 1 | 0.8 | 3 | 2.3 | 1 | 0.8 | 102 | 79.7 |
| Head and neck | 111 | 0 | 0.0 | 6 | 5.4 | 72 | 64.9 | 0 | 0.0 | 0 | 0.0 | 4 | 3.6 | 0 | 0.0 | 37 | 33.3 |
| squamous | | | | | | | | | | | | | | | | | |
| carcinoma | | | | | | | | | | | | | | | | | |
| Low-grade glioma | 95 | 0 | 0.0 | 1 | 1.1 | 7 | 7.4 | 0 | 0.0 | 0 | 0.0 | 0 | 0.0 | 0 | 0.0 | 87 | 91.6 |
| Small cell lung | 75 | 1 | 1.3 | 4 | 5.3 | 10 | 13.3 | 0 | 0.0 | 0 | 0.0 | 2 | 2.7 | 0 | 0.0 | 62 | 82.7 |
| cancer | | | | | | | | | | | | | | | | | |
| Liver hepatocellular | 73 | 2 | 2.7 | 1 | 1.4 | 7 | 9.6 | 1 | 1.4 | 0 | 0.0 | 0 | 0.0 | 0 | 0.0 | 64 | 87.7 |
| carcinoma | | | | | | | | | | | | | | | | | |
| Small intestinal | 72 | 6 | 8.3 | 6 | 8.3 | 12 | 16.7 | 5 | 6.9 | 1 | 1.4 | 1 | 1.4 | 1 | 1.4 | 54 | 75.0 |
| malignancies | | | | | | | | | | | | | | | | | |
| Other female genital | 57 | 1 | 1.8 | 4 | 7.0 | 27 | 47.4 | 1 | 1.8 | 0 | 0.0 | 3 | 5.3 | 0 | 0.0 | 29 | 50.9 |
| tract malignancies | | | | | | | | | | | | | | | | | |
| Non-epithelial | 56 | 1 | 1.8 | 0 | 0.0 | 4 | 7.1 | 0 | 0.0 | 0 | 0.0 | 0 | 0.0 | 0 | 0.0 | 51 | 91.1 |
| ovarian cancer | | | | | | | | | | | | | | | | | |
| Gastrointestinal | 52 | 0 | 0.0 | 0 | 0.0 | 20 | 38.5 | 0 | 0.0 | 0 | 0.0 | 0 | 0.0 | 0 | 0.0 | 32 | 61.5 |
| stromal tumors | | | | | | | | | | | | | | | | | |
| (GIST) | | | | | | | | | | | | | | | | | |
| Uveal melanoma | 50 | 1 | 2.0 | 1 | 2.0 | 10 | 20.0 | 1 | 2.0 | 1 | 2.0 | 1 | 2.0 | 1 | 2.0 | 40 | 80.0 |
| Retroperitoneal or | 46 | 0 | 0.0 | 0 | 0.0 | 10 | 21.7 | 0 | 0.0 | 0 | 0.0 | 0 | 0.0 | 0 | 0.0 | 36 | 78.3 |
| peritoneal sarcoma | | | | | | | | | | | | | | | | | |
| Thyroid carcinoma | 42 | 1 | 2.4 | 1 | 2.4 | 26 | 61.9 | 1 | 2.4 | 1 | 2.4 | 1 | 2.4 | 1 | 2.4 | 16 | 38.1 |
| Extrahepatic bile | 29 | 1 | 3.4 | 1 | 3.4 | 6 | 20.7 | 1 | 3.4 | 1 | 3.4 | 1 | 3.4 | 1 | 3.4 | 23 | 79.3 |
| duct | | | | | | | | | | | | | | | | | |
| adenocarcinoma | | | | | | | | | | | | | | | | | |
| Lymphoma | 27 | 0 | 0.0 | 2 | 7.4 | 16 | 59.3 | 0 | 0.0 | 0 | 0.0 | 2 | 7.4 | 0 | 0.0 | 11 | 40.7 |
| Thymic carcinoma | 26 | 0 | 0.0 | 1 | 3.8 | 18 | 69.2 | 0 | 0.0 | 0 | 0.0 | 1 | 3.8 | 0 | 0.0 | 8 | 30.8 |
| Male genital tract | 15 | 0 | 0.0 | 0 | 0.0 | 3 | 20.0 | 0 | 0.0 | 0 | 0.0 | 0 | 0.0 | 0 | 0.0 | 12 | 80.0 |
| malignancy | | | | | | | | | | | | | | | | | |
| Multiple myeloma | 10 | 0 | 0.0 | 0 | 0.0 | 0 | 0.0 | 0 | 0.0 | 0 | 0.0 | 0 | 0.0 | 0 | 0.0 | 10 | 100.0 |
| Retroperitoneal or | 7 | 0 | 0.0 | 0 | 0.0 | 2 | 28.6 | 0 | 0.0 | 0 | 0.0 | 0 | 0.0 | 0 | 0.0 | 5 | 71.4 |
| peritoneal | | | | | | | | | | | | | | | | | |
| carcinoma | | | | | | | | | | | | | | | | | |
| Merkel cell | 6 | 0 | 0.0 | 2 | 33.3 | 0 | 0.0 | 0 | 0.0 | 0 | 0.0 | 0 | 0.0 | 0 | 0.0 | 4 | 66.7 |
| carcinoma | | | | | | | | | | | | | | | | | |
| Nodal diffuse large | 5 | 0 | 0.0 | 0 | 0.0 | 2 | 40.0 | 0 | 0.0 | 0 | 0.0 | 0 | 0.0 | 0 | 0.0 | 3 | 60.0 |
| B-cell lymphoma | | | | | | | | | | | | | | | | | |
| Malignant solitary | 3 | 0 | 0.0 | 0 | 0.0 | 0 | 0.0 | 0 | 0.0 | 0 | 0.0 | 0 | 0.0 | 0 | 0.0 | 3 | 100.0 |
| fibrous tumor of the | | | | | | | | | | | | | | | | | |
| pleura | | | | | | | | | | | | | | | | | |
| Acute myeloid | 1 | 0 | 0.0 | 0 | 0.0 | 0 | 0.0 | 0 | 0.0 | 0 | 0.0 | 0 | 0.0 | 0 | 0.0 | 1 | 100.0 |
| leukemia | | | | | | | | | | | | | | | | | |
| Lung | 1 | 0 | 0.0 | 0 | 0.0 | 0 | 0.0 | 0 | 0.0 | 0 | 0.0 | 0 | 0.0 | 0 | 0.0 | 1 | 100.0 |
| bronchioloalveolar | | | | | | | | | | | | | | | | | |
| carcinoma | | | | | | | | | | | | | | | | | |

Certain cancer types showed interesting relationships regarding MSI and TMB. See FIG. 32J, which show scatter plots comparing MSI as altered microsatellite (MS) loci determined by NGS to TMB per megabase for colorectal adenocarcinoma (panel i; n=1267), endometrial cancer (panel ii; n=667), NSCLC (panel iii; n=964), and melanoma (panel iv; n=175). The horizontal line indicates 46 altered MS and the vertical line indicates 17 mutations/Mb, which are the cutoff used to determine high status. In both CRC and endometrial cancer, the majority of MSI-H cases were also high in TMB. This pattern was not seen in two cancer types driven primarily by environmentally caused mutagenesis, NSCLC and melanoma. In NSCLC, 14.0% (264/1868) of cases were high TMB, but only 0.6% (12/1868) were MSI-H. Melanoma had no cases that were MSI-H, but 36.5% were high TMB (126/345).

Detailed patient characteristics and results for all samples used in this Example can be found in “Table 55 —Patient Characteristics and Test Results” of priority document U.S. Provisional Patent Ser. No. 62/631,381, filed Feb. 15, 2018, which application is incorporated by reference in its entirety, including without limitation Table 55 therein.

### DISCUSSION

MSI-H cancers are a genetically-defined subset of cancers with the potential for enhanced responsiveness to anti-PD-1 therapies and related therapies. [5-7] Determining MSI status across cancer types offers the opportunity to identify patients who are likely to respond to such treatments, while avoiding unnecessary toxicities for patients identified as unlikely to respond. In this Example, we developed a sensitive and specific MSI assay by NGS that is comparable to the existing gold standard of PCR FA methods without requiring matched samples from normal tissue.

The method was calibrated using 2,189 cases across 26 cancer types that had both MSI-FA and 592-gene NGS results. This number of matched samples between FA and NGS is a substantially larger calibration set that that used in another published NGS MSI method. [22] Previously published data using the MSI-NGS method described herein found MSI-H status present in 24 of 31 cancer types. [23]Likewise, here we identified MSI-H in 23 of 26 cancer types. The detection of MSI-H cases in this extensive list of cancer types supports the concept that MSI is a generalized cancer phenotype. [3]

Notably, MSI-H cases that were not TMB-H or PD-L1-positive occurred in significant percentages of ovarian (24%), neuroendocrine (57%), and cervical (33%) cancers. With the recent approval of pembrolizumab for MSI-H patients of any solid tumor type, this subset of patients now has a promising treatment that would not have been identified using either of the other two immunotherapy biomarker assays. Given the lack of overlap of MSI and high TMB in several cancer types, these data suggest patient benefit by performing both TMB analysis and MSI-NGS and potentially other complementary tests, e.g., PD-L1 IHC.

This MSI-NGS assay has concordance with the FA method for CRC (100% sensitivity and 99.9% specificity) but slightly reduced agreement when looking across all cancer types (95.8% sensitivity and 99.9% specificity; PPV of 94.5%). As the FA test was developed for CRC, MSI-NGS discrepancies in non-CRC cancer types may be due to other loci being involved in these cancer types that are not measured by the FA method. Without being bound by theory, this raises the possibility that some of the FA PCR results could be false negatives, rather than the corresponding MSI-NGS results being false positives. For example, our NGS assay has broader microsatellite coverage and may be a better predictor of response than the FA assay, which is limited to 5 microsatellite sites.

The use of NGS to determine MSI status offers advantages over FA by PCR. Due to the large number of microsatellite regions analyzed, this method of NGS analysis of MSI does not require a sample of normal tissue for comparison. The comparison of a large number of microsatellite sequences to a reference human genome was able to provide a level of sensitivity comparable to that achieved using only a few microsatellites and comparing to a normal sample from the same patient. Thus, with this method, it is feasible to determine MSI status for patients who do not have available normal tissue or for whom it would be a burden to obtain. Coupling the calculation of MSI to data that are already generated by a broad NGS sequencing panel allows for MSI status to be determined efficiently for any patient who is already receiving broad NGS sequencing results, without adding the cost of an additional stand-alone test or consuming additional tumor tissue that could be used for other testing. Further, while FA by PCR was optimized to analyze CRC, [24] our NGS analysis of MSI is a pan-cancer method whose development was technically validated across 26 cancer types.

IHC testing for MMR protein is commonly performed on CRC and endometrial cancer cases to test for Lynch syndrome. Clinical evidence indicates that treatments with the PD-1 inhibitors pembrolizumab and nivolumab both lead to favorable responses in patients with dMMR tumors. [5,7,18]Our NGS-MSI assay has 87.1% sensitivity for dMMR detection compared to MMR-IHC (see Table 18). However, the proteins measured by standard MMR-IHC (MLH1, MSH2, MSH6, and PMS2) are not equal in their contribution to the mismatch repair process. Previous research on endometrial carcinoma found that most MSI-H tumors had loss of MLH1 and PMS2, with concordant loss of the MLH1/PMS2 heterodimer in 48% and with MSI-H in 97% of PSM2-negative cases. [25] Without being bound by theory, there may be a subset of dMMR cases with relatively low microsatellite alterations, which are identified as MSS by NGS, that have lower rates of response to PD-1 inhibition compared with cases that are MSI-H and dMMR cases. This is supported by data indicating that the subset of dMMR CRC cases called MSS by FA were less likely to respond to nivolumab than MSI-H cases. [18] These data suggest potential benefit of both MSI-NGS and MMR-IHC, in cancer types where MMR-IHC loss is more common, to identify more patients with potential response.

Current NCCN guidelines recommend MSJ and MMR proficiency testing on patients with colon and endometrial cancer. Considering the landscape of the site-agnostic approval of pembrolizumab for patients with MSI-H cancers, the testing recommendation should now be expanded to include all patients with advanced solid tumors lacking satisfactory treatment options. The method of MSI-NGS presented in this Example addresses disadvantages of both FA and MMR-IHC, thus providing an improved platform to measure MSI status in all tumors. MSI-NGS can be added to other malignancy-specific molecular panels, requires no extra tissue, and has lower marginal cost when FA is considered as an add-on test that must be performed along with an NGS panel. With the evolution in cancer care toward molecularly-defined diagnoses, validation of NGS measurement of MSI status provides a mechanism for all cancer patients, regardless of malignancy, to achieve testing that can determine whether a potentially life-extending agent may be appropriate.

We also compared MSI with TMB. See, e.g., FIG. 32C. MSI is measured by NGS through counting insertions or deletions of 2-5 nucleotides in specific areas of the genome known to accumulate errors in microsatellites. In contrast, TMB was measured here by counting nonsynonymous mutations across the sequenced portion of the genome. Therefore, TMB can capture a wider range of mutational signatures because it covers the genome more broadly. Although most MSI-H cases are high TMB, the opposite is not true. Our cut-off for high TMB of >17 mutations/Mb is similar to the recently published cutoff values of >13.8 and >20 mutations/Mb. [6,26] True biological differences in TML and MSI appear to exist in certain cancer types. For example, tumors driven primarily by environmentally caused mutations (e.g., NSCLC and melanoma) have a higher proportion of cases with high TMB vs MSI (FIG. 32C) compared to tumors that are not as strongly associated with environmental factors (e.g., smoking and sun exposure, respectively).

The 11,348 cases included in these comprehensive genomic analyses by NGS are generally from patients with advanced, refractory disease who lacked obvious treatment options. This could lead to some downward bias in the reported MSI frequencies, e.g., CRC MSI-H rates are lower in advanced disease than in the overall CRC population. [4] Thus, a larger percentage of patients may benefit from MSI-NGS testing than even suggested here.

In conclusion, we used a large patient database to develop a method to determine MSI status using NGS. The MSI-NGS test is applicable across cancer types and does not require matched normal samples, which is particularly beneficial for patients where such tissue is limited or not available. The investigation of the relationship among TMB, MSI, and PD-L1 revealed a population with MSI-H disease, but low TMB and no PD-L1 expression, thus expanding the pool of potential immunotherapy recipients. Without being bound by theory, the best option may be to measure all three to ensure that as many patients as possible benefit from these drugs.

### REFERENCES

1. de la Chapelle A, Hampel H. Clinical relevance of microsatellite instability in colorectal cancer. J Clin Oncol. 2010; 28(20):3380-7.
2. Murphy K M, Zhang S, Geiger T, Hafez M J, Bacher J, Berg K D, et al. Comparison of the microsatellite instability analysis system and the Bethesda panel for the determination of microsatellite instability in colorectal cancers. J Mol Diagn. 2006; 8(3):305-11.
3. Hause R J, Pritchard C C, Shendure J, Salipante S J. Classification and characterization of microsatellite instability across 18 cancer types. Nat Med. 2016; 22(11):1342-50.
4. Lee V, Murphy A, Le D T, Diaz L A, Jr. Mismatch repair deficiency and response to immune checkpoint blockade. The Oncologist. 2016; 21(10):1200-11.
5. Le D T, Durham J N, Smith K N, Wang H, Bartlett B R, Aulakh L K, et al. Mismatch repair deficiency predicts response of solid tumors to P D-1 blockade. Science. 2017; 357(6349):409-13.
6. Zehir A, Benayed R, Shah R H, Syed A, Middha S, Kim H R, et al. Mutational landscape of metastatic cancer revealed from prospective clinical sequencing of 10,000 patients. Nat Med. 2017; 23(6):703-13.
7. Le D T, Uram J N, Wang H, Bartlett B R, Kemberling H, Eyring A D, et al. P D-1 blockade in tumors with mismatch-repair deficiency. N Engl J Med. 2015; 372(26):2509-20.
8. Overman M, Kopetz S, McDermott R, Leach J, Lonardi S, Lenz H, et al. Nivolumab+ipilimumab in treatment of patients with metastatic colorectal cancer with and without high microsatellite instability (MSI-H): CheckMate-142 interim results. J Clin Oncol. 2016;34:suppl; abstract 3501.
9. Bouffet E, Larouche V, Campbell B B, Merico D, de Borja R, Aronson M, et al. Immune checkpoint Inhibition for hypermutant glioblastoma multiforme resulting from germline biallelic mismatch repair deficiency. J Clin Oncol. 2016; 34(19):2206-11.
10. Castro M P, Goldstein N. Mismatch repair deficiency associated with complete remission to combination programmed cell death ligand immune therapy in a patient with sporadic urothelial carcinoma: immunotheranostic considerations. J Immunother Cancer. 2015; 3:58.
11. Rizvi N A, Hellmann M D, Snyder A, Kvistborg P, Makarov V, Havel J J, et al. Cancer immunology. Mutational landscape determines sensitivity to P D-1 blockade in non-small cell lung cancer. Science. 2015; 348(6230):124-8.
12. Rosenberg J E, Hoffman-Censits J, Powles T, van der Heijden M S, Balar A V, Necchi A, et al. Atezolizumab in patients with locally advanced and metastatic urothelial carcinoma who have progressed following treatment with platinum-based chemotherapy: a single-arm, multicentre, phase 2 trial. Lancet. 2016; 387(10031):1909-20.
13. Snyder A, Makarov V, Merghoub T, Yuan J, Zaretsky J M, Desrichard A, et al. Genetic basis for clinical response to CTLA-4 blockade in melanoma. N Engl J Med. 2014; 371(23):2189-99.
14. Patel S P, Kurzrock R. P D-L1 expression as a predictive biomarker in cancer immunotherapy. Mol Cancer Ther. 2015; 14(4):847-56.
15. Borghaei H, Paz-Ares L, Horn L, Spigel D R, Steins M, Ready N E, et al. Nivolumab versus docetaxel in advanced nonsquamous non-small-cell lung cancer. N Engl J Med. 2015; 373(17):1627-39.
16. Garon E B, Rizvi N A, Hui R, Leighl N, Balmanoukian A S, Eder J P, et al. Pembrolizumab for the treatment of non-small-cell lung cancer. N Engl J Med. 2015; 372(21):2018-28.
17. Taube J M, Klein A, Brahmer J R, Xu H, Pan X, Kim J H, et al. Association of P D-1, P D-1 ligands, and other features of the tumor immune microenvironment with response to anti-P D-1 therapy. Clin Cancer Res. 2014; 20(19):5064-74.
18. Overman M J, McDermott R, Leach J L, Lonardi S, Lenz H J, Morse M A, et al. Nivolumab in patients with metastatic DNA mismatch repair-deficient or microsatellite instability-high colorectal cancer (CheckMate 142): an open-label, multicentre, phase 2 study. Lancet Oncol. 2017.
19. Zhang L. Immunohistochemistry versus microsatellite instability testing for screening colorectal cancer patients at risk for hereditary nonpolyposis colorectal cancer syndrome. Part I I. The utility of microsatellite instability testing. J Mol Diagn. 2008; 10(4):301-7.
20. Auton A, Brooks L D, Durbin R M, Garrison E P, Kang H M, Korbel J O, et al. A global reference for human genetic variation. Nature. 2015; 526(7571):68-74.
21. Stadler Z K, Battaglin F, Middha S, Hechtman J F, Tran C, Cercek A, et al. Reliable detection of mismatch repair deficiency in colorectal cancers using mutational load in next-generation sequencing panels. J Clin Oncol. 2016; 34(18):2141-7.
22. Hall M J, Gowen K, Sanford E M, Elvin J A, Ali S M, Kaczmar J, et al. Evaluation of microsatellite instability (MSI) status in 11,573 diverse solid tumors using comprehensive genomic profiling (CGP). J Clin Oncol. 2017;34(suppl):abst 1523.
23. Le D T, Durham J N, Smith K N, Wang H, Bartlett B R, Aulakh L K, et al. Mismatch-repair deficiency predicts response of solid tumors to P D-1 blockade. Science. 2017.
24. Bacher J W, Flanagan L A, Smalley R L, Nassif N A, Burgart L J, Halberg R B, et al. Development of a fluorescent multiplex assay for detection of MSI-high tumors. Dis Markers. 2004;20(4-5):237-50.
25. Modica I, Soslow R A, Black D, Tornos C, Kauff N, Shia J. Utility of immunohistochemistry in predicting microsatellite instability in endometrial carcinoma. Am J Surg Pathol. 2007; 31(5):744-51.
26. Chalmers Z R, Connelly C F, Fabrizio D, Gay L, Ali S M, Ennis R, et al. Analysis of 100,000 human cancer genomes reveals the landscape of tumor mutational burden. Genome Med. 2017;9(1):34.

The above references are denoted by bracketed numbers in the Example. Each of these references is incorporated by reference herein in its entirety.

Although preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. (canceled)

2. A method of determining microsatellite instability (MSI) in a biological sample of a patient, the method comprising:
(a) obtaining a nucleic acid sequence for each of a plurality of microsatellite loci from the biological sample, thereby obtaining nucleic acid sequences;
(b) determining a number of altered microsatellite loci based on the nucleic acid sequences obtained in step (a);
(c) comparing the number of altered microsatellite loci determined in step (b) to a threshold number that is selected based on a patient characteristic of the patient;
(d) identifying the biological sample as MSI-high when the number of altered microsatellite loci determined in step (b) is greater than or equal to the threshold number; and
(e) administer immunotherapy to the patient based on the biological sample being MSI-high.

3. The method of claim 2, wherein obtaining the nucleic acid sequences comprises sequencing nucleic acids from the biological sample.

4. The method of claim 2, wherein the sequencing is of genomic DNA.

5. The method of claim 3, wherein sequence reads are obtained from the sequencing, and wherein obtaining the nucleic acid sequence further comprises:
constructing the nucleic acid sequence by identifying overlapping sequences in different sequence reads.

6. The method of claim 2, wherein the threshold number is calibrated based on a comparison of the number of altered microsatellite loci per patient to MSI results obtained using a different laboratory technique than used in step (a) on a same biological sample from each patient of a plurality of patients.

7. The method of claim 6, wherein the different laboratory technique comprises fragment analysis.

8. The method of claim 2, wherein determining the number of altered microsatellite loci in step (b) comprises comparing each nucleic acid sequence obtained in step (a) to a reference sequence for each microsatellite loci.

9. The method of claim 2, wherein determining the number of altered microsatellite loci comprises identifying insertions or deletions that increase or decrease a number of repeats in each microsatellite loci.

10. The method of claim 9, wherein the number of altered microsatellite loci only counts each altered loci once regardless of the number of insertions or deletions at that loci.

11. The method of claim 2, wherein the threshold number is calibrated across at least 2, 3, 4, 5, 6, 7, 8, 9, 10, 11, 12, 13, 14, 15, 20, or 25 distinct cancer lineages using sensitivity, specificity, positive predictive value, negative predictive value, or any combination thereof.

12. The method of claim 2, further comprising identifying the biological sample as microsatellite stable (MSS) when the number of altered microsatellite loci is below the threshold number.

13. The method of claim 2, further comprising identifying the biological sample as MSI-low if the number of altered microsatellite loci in the biological sample is less than or equal to a lower threshold number than the threshold number.

14. The method of claim 2, wherein the biological sample comprises formalin-fixed paraffin-embedded (FFPE) tissue, fixed tissue, a core needle biopsy, a fine needle aspirate, unstained slides, fresh frozen (FF) tissue, formalin samples, tissue comprised in a solution that preserves nucleic acid or protein molecules, a fresh sample, a malignant fluid, a bodily fluid, a tumor sample, a tissue sample, or any combination thereof.

15. The method of claim 2, wherein the plurality of microsatellite loci excludes: i) sex chromosome loci; ii) microsatellite loci in regions that typically have lower coverage depth relative to other genomic regions; iii) microsatellites with repeat unit lengths greater than 3, 4, 5, 6 or 7 nucleotides; or iv) any combination of i)-iii).

16. The method of claim 2, further comprising:
comprises determining a tumor mutation burden for the biological sample; and
identifying the biological sample as TMB-high when the tumor mutation burden is greater than or equal to a TMB threshold.

17. The method of claim 16, wherein the tumor mutation burden is measured as a number of missense mutations per megabase.

18. The method of claim 2, further comprising: identifying, based on whether the biological sample is MSI-high, at least one of: A) at least one therapy with potential benefit for treatment of a cancer; B) at least one therapy with potential lack of benefit for treatment of the cancer; and C) at least one therapy associated with a clinical trial.

19. The method of claim 2, wherein the patient characteristic indicates a demography of the patient.

20. The method of claim 2, wherein the plurality of microsatellite loci include dinucleotide or higher repeats.

21. The method of claim 2, wherein the plurality of microsatellite loci are selected from Table 16.

22. The method of claim 2, each of the plurality of microsatellite loci is located within a vicinity of a cancer gene selected from Table 7, Table 8, Table 9, Table 10, or any combination thereof.
